(19) United States
(12) Patent Application Publication
(10) Pub. No.: US 2025/0205167 A1
(43) Pub. Date: Jun. 26, 2025

(54) NOVEL IONIZABLE LIPIDS AND LIPID NANOPARTICLES AND METHODS OF USING THE SAME

(71) Applicant: SAIL BIOMEDICINES, INC., Cambridge, MA (US)

(72) Inventors: Alessandra BARTOLOZZI, Cambridge, MA (US); John PROUDFOOT, Cambridge, MA (US); Arijit ADHIKARI, Cambridge, CA (US); Siddharth PATEL, Cambridge, MA (US); Alaina HOWE, Cambridge, MA (US); Dominick SALERNO, Cambridge, MA (US); Jennifer UNION, Cambridge, MA (US); Sanmit ADHIKARI, Cambridge, MA (US); Roman ERDMANN, Cambridge, MA (US)

(21) Appl. No.: 18/850,325

(22) PCT Filed: Mar. 24, 2023
(86) PCT No.: PCT/US2023/016300

§ 371 Date: Sep. 24, 2024

Related U.S. Application Data
(60) Provisional application No. 63/323,948, filed on Mar. 25, 2022.

(51) Int. Cl. A61K 9/51; A61K 31/7105; C07D 207/12; C07D 207/16; C07D 211/60; C07D 403/12

(52) U.S. Cl. CPC A61K 9/5123; A61K 31/7105; C07D 207/12; C07D 207/16; C07D 211/60; C07D 403/12

(57) ABSTRACT

The disclosure relates to novel ionizable lipids and lipid compositions and pharmaceutical compositions comprising these ionizable lipids that can be used in the delivery of therapeutic agent.

### CROSS-REFERENCE TO RELATED APPLICATIONS

This application claims benefit of priority to U.S. Provisional Application No. 63/323,948 filed Mar. 25, 2022, which is herein incorporated by reference in its entirety.

### BACKGROUND

Lipid nanoparticles (“LNPs”) formed from ionizable amine-containing lipids can serve as therapeutic cargo vehicles for delivery of biologically active agents, such as coding RNAs (i.e., messenger RNAs (mRNAs), guide RNAs) and non-coding RNAs (i.e. antisense, siRNA), into cells. LNPs can facilitate delivery of oligonucleotide agents across cell membranes and can be used to introduce components and compositions into living cells.

Biologically active agents that are particularly difficult to deliver to cells include proteins, nucleic acid-based drugs, and derivatives thereof, particularly drugs that include relatively large oligonucleotides, such as mRNA or guide RNA. Compositions for delivery of promising mRNA therapy or editing technologies into cells, such as for delivery of CRISPR/Cas9 system components, have become of particular interest.

With the advent of the recent pandemic, messenger RNA therapy has become an increasingly important option for treatment of various diseases, including for viral infectious diseases and for those associated with deficiency of one or more proteins. Compositions with useful properties for in vitro and in vivo delivery that can stabilize and/or deliver RNA components, have also become of particular interest.

There thus continues to be a need in the art for novel lipid compounds to develop lipid nanoparticles or other lipid delivery mechanisms for therapeutics delivery. This invention answers that need.

### SUMMARY OF THE INVENTION

Disclosed herein are novel ionizable lipids that can be used in combination with at least one other lipid component, such as neutral lipids, cholesterol, and polymer conjugated lipids, to form lipid nanoparticle compositions. The lipid nanoparticle compositions may be used to facilitate the intracellular delivery of therapeutic nucleic acids in vitro and/or in vivo.

Disclosed herein are ionizable amine-containing lipids useful for formation of lipid nanoparticle compositions. Such LNP compositions may have properties advantageous for delivery of nucleic acid cargo, such as delivery of coding and non-coding RNAs to cells.

Methods for treatment of various diseases or conditions, such as those caused by infectious entities and/or insufficiency of a protein, using the disclosed lipid nanoparticles are also provided.

Disclosed below are ionizable lipids of various formulas, including, e.g., Formulas (I), (IA-1), (IA-2), (IIA)-(IIC), (IIA-1), (IIIA)-(IIIIE), (IIIC-1), (IVA-1)-(IVA-3), (IVC-1)-(IVC-2), (VC-1)-(VC-6).

One aspect of the invention relates to a compound of Formula (I):

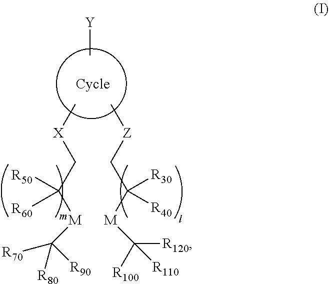

a pharmaceutically acceptable salt thereof, or a stereoisomer of any of the foregoing, wherein:

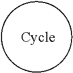

is cyclic or heterocyclic moiety;
Y is alkyl, hydroxy, hydroxyalkyl or

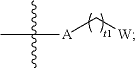

A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
each of X and Z is independently absent, —O—, —CO—, —N(R⁷)—, —O-alkylene-; -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, hydroxyalkyl, or aminoalkyl;
each M is independently a biodegradable moiety;
each of R₃₀, R₄₀, R₅₀, R₆₀, R₇₀, R₈₀, R₉₀, R₁₀₀, R₁₁₀, and R₁₂₀ is independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
each of l and m is an integer from 1 to 10;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl.

In some embodiments, disclosed are ionizable lipids of Formula (I):

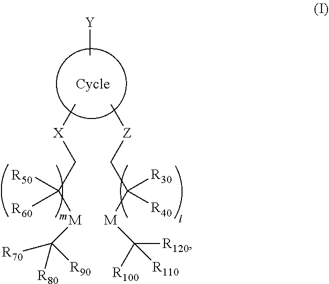

a pharmaceutically acceptable salt thereof, or a stereoisomer of any of the foregoing, wherein:

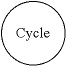

is cyclic or heterocyclic moiety;
Y is alkyl, hydroxy, hydroxyalkyl,

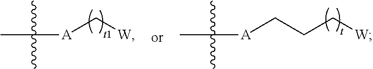

A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
each of X and Z is independently absent, —O—, —N(R⁷)—, —O-alkylene-; -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, hydroxyalkyl, or aminoalkyl;
each M is independently a biodegradable moiety;
each of R₃₀, R₄₀, R₅₀, R₆₀, R₇₀, R₈₀, R₉₀, R₁₀₀, R₁₁₀, and R₁₂₀ is independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
each of l and m is an integer from 1 to 10;
t is 0, 1, 2, or 3;
t1 is an integer from 0 to 10; and
W is hydroxyl, hydroxyalkyl, or one of the following moieties:

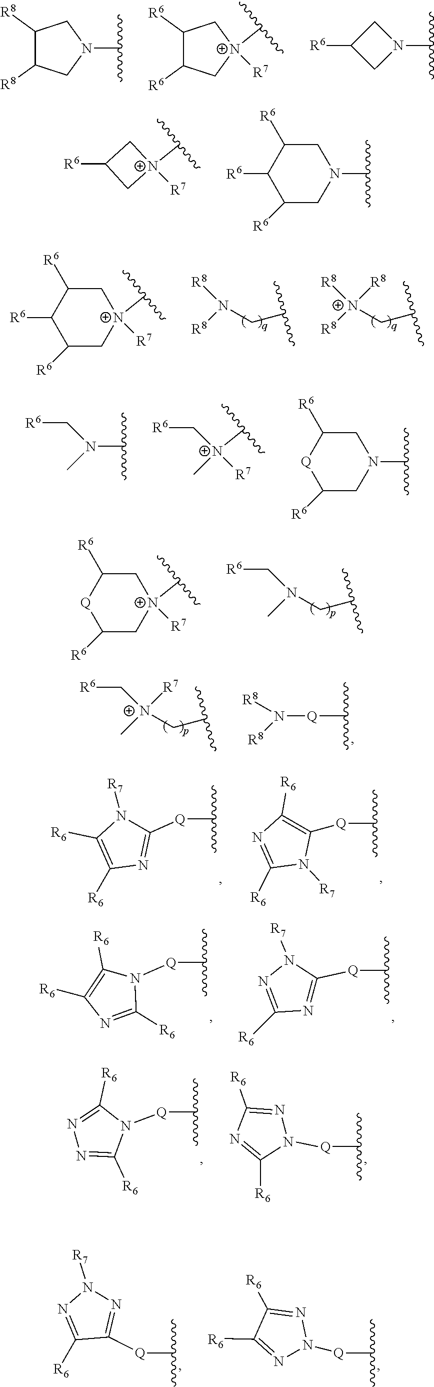

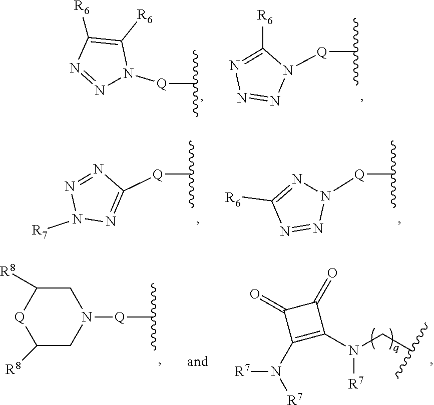

each Q is independently absent, —O—, —C(O)—, —C(S)—, —C(O)O—, —C(R⁷)₂—, —C(O)N(R⁷)—, —C(S)N(R⁷)—, or —N(R⁷);
R⁶ is independently H, alkyl, hydroxyl, hydroxyalkyl, alkoxy, amino, aminoalkyl, alkylamino, thiol, thiolalkyl, or N⁺(R⁷)₃-alkylene-Q-;
each R⁸ is independently H, alkyl, hydroxyalkyl, amino, aminoalkyl, thiol, or thiolalkyl, or two R⁸ together with the nitrogen atom may form a ring;
q is 0, 1, 2, 3, 4, or 5; and
p is 0, 1, 2, 3, 4, or 5.

In some embodiments, Y is hydroxyl,

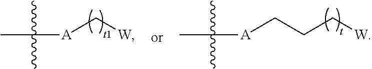

In some embodiments, disclosed are ionizable lipids of Formula (IA-1) or (IA-2):

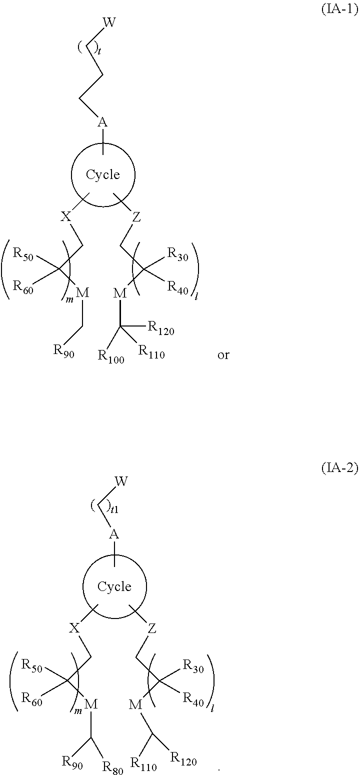

a pharmaceutically acceptable salt thereof, or a stereoisomer of any of the foregoing,
wherein

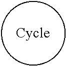

is cyclic or heterocyclic moiety;
A is absent, —O—, —N(R⁷)—, —O-alkylene-, -alkylene-O—, —OC(O)—, —C(O)O—, —N(R⁷)C(O)—, —C(O)N(R⁷)—, N(R⁷)C(O)NH—, —S—, —S—S—, or a bivalent heterocycle;
X is absent, —O—, —C(O), —N(R⁷)—, —O-alkylene-; -alkylene-O—, —OC(O)—, —C(O)O—, —N(R⁷)C(O)—, —C(O)N(R⁷)—, or —S—;
Z is absent, —O—, —N(R⁷)—, —O-alkylene-; -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxyalkyl, or aminoalkyl;
each M is independently a biodegradable moiety;
each of R₃₀, R₄₀, R₅₀, R₆₀, R₁₀₀, R₁₁₀ and R₁₂₀ is independently H, C₁-C₁₆ branched or unbranched alkyl or C₁-C₁₆ branched or unbranched alkenyl, optionally interrupted with heteroatom or substituted with OH, SH, or halogen;
R₉₀ is C₁-C₁₅ branched or unbranched alkyl, C₁-C₁₅ branched or unbranched alkenyl, or cycloalkyl or substituted cycloalkyl; and
t is 0, 1, 2, or 3;
t1 is an integer from 0 to 10;
l is an integer from 1 to 10;
m is an integer from 1 to 10; and
W is hydroxyl, hydroxyalkyl, or one of the following moieties:

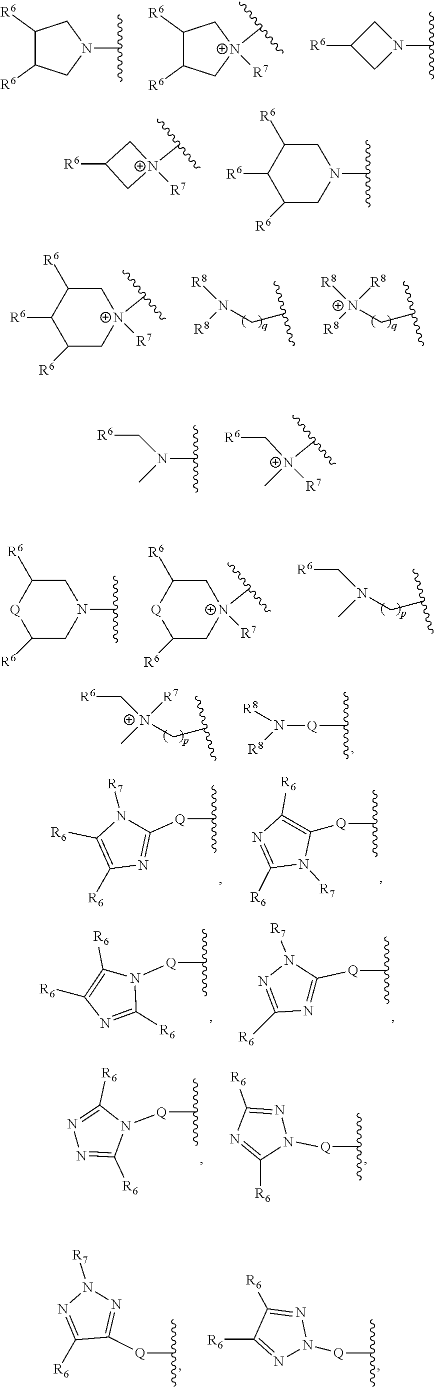

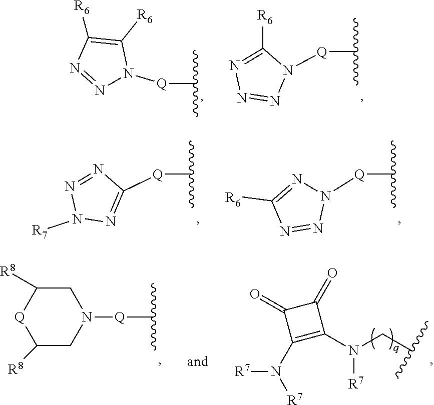

wherein
Q is —O—, —C(O)—, —C(S)—, —C(O)O—, —C(R⁷)₂—, —C(O)N(R⁷)—, —C(S)N(R⁷)—, or —N(R⁷);
each R⁶ is independently H, alkyl, hydroxyl, hydroxyalkyl, alkoxy, amino, aminoalkyl, alkylamino, thiol, thiolalkyl, or N⁺(R⁷)₃-alkylene-Q-;
each R⁸ is independently H, alkyl, hydroxyalkyl, amino, aminoalkyl, thiol, or thiolalkyl, or two R⁸ together with the nitrogen atom may form a ring;
q is 0, 1, 2, 3, 4, or 5; and
p is 0, 1, 2, 3, 4, or 5.

In some embodiments,

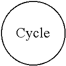

has a structure of formula

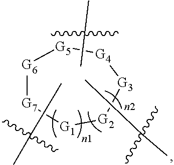

wherein:
each of G₁, G₂, G₃, G₄, G₅, G₆, and G₇ is independently C(R′)(R″), O, or N, provided that no more than two of G₁-G₇ are O or N;
R′ and R″ are each independently absent, H, alkyl, or two R′ from the two neighboring G together form a second 5- to 7-membered cyclic or heterocylic ring; and
n₁ and n₂ are each independently 0 or 1.

In some embodiments,

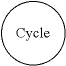

is selected from pyrrolidine, piperidine, piperazine, cyclohexane, cyclopentane, tetrahydrofuran; tetrahydropyran; morpholine, and dioxane.

In some embodiments,

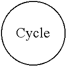

is a 5- to 7-membered, monocyclic ring. In some embodiments,

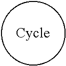

is a 5- to 7-membered, monocyclic, cycloalkane ring. In some embodiments,

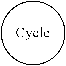

is a 5- to 7-membered, monocyclic, heterocycle ring.

In some embodiments,

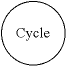

is a bicyclic or tricyclic ring, i.e., containing two or more rings, such as fused rings.

In some embodiments, X is absent, —O—, or —C(O)—.

In some embodiments, Z is —O—, —C(O)O—, or —OC(O)—.

In some embodiments, each of R₃₀, R₄₀, R₅₀, and R₆₀ is H or C₁-C₄ branched or unbranched alkyl.

In some embodiments, each of R₃₀, R₄₀, R₅₀, and R₆₀ is H.

In some embodiments, each of R₇₀ and R₈₀ is H; and R₉₀ is C₁-C₁₅ branched or unbranched alkyl, C₁-C₁₅ branched or unbranched alkenyl, cycloalkyl or substituted cycloalkyl. In some embodiments, R₉₀ is C₁-C₁₅ branched or unbranched alkyl, C₁-C₁₅ branched or unbranched alkenyl. In some embodiments, R₉₀ is C₁-C₁₅ branched or unbranched alkyl. In some embodiments R₉₀ is C₁-C₁₂ branched or unbranched alkyl.

In some embodiments, R₇₀ is H; and each of R₈₀ and R₉₀ is independently C₁-C₁₅ branched or unbranched alkyl, C₁-C₁₅ branched or unbranched alkenyl, or cycloalkyl or substituted cycloalkyl. In some embodiments, each of R₈₀ and R₉₀ is independently C₁-C₁₅ branched or unbranched alkyl, C₁-C₁₅ branched or unbranched alkenyl. In some embodiments, each of R₈₀ and R₉₀ is independently C₁-C₁₅ branched or unbranched alkyl. In some embodiments, each of R₈₀ and R₉₀ is independently C₁-C₁₂ branched or unbranched alkyl. In some embodiments, each of R₈₀ and R₉₀ is independently C₁-C₈ branched or unbranched alkyl.

In some embodiments, R₁₀₀ is H; and each of R₁₁₀ and R₁₂₀ is independently C₁-C₁₅ branched or unbranched alkyl, C₁-C₁₅ branched or unbranched alkenyl, or cycloalkyl or substituted cycloalkyl. In some embodiments, each of R₁₁₀ and R₁₂₀ is independently C₁-C₁₅ branched or unbranched alkyl, C₁-C₁₅ branched or unbranched alkenyl. In some embodiments, each of R₁₁₀ and R₁₂₀ is independently C₁-C₁₅ branched or unbranched alkyl. In some embodiments, each of R₁₁₀ and R₁₂₀ is independently C₁-C₁₂ branched or unbranched alkyl. In some embodiments, each of R₁₁₀ and R₁₂₀ is independently C₁-C₈ branched or unbranched alkyl.

In some embodiments, l is from 3 to 10, from 3 to 7, or from 4 to 7. In some embodiments, l is 4, 5, 6, 7, 8, 9 or 10. In some embodiments, l is 4, 5, 6, or 7.

In some embodiments, m is from 4 to 10, from 5 to 8, from 1 to 7, from 3 to 7, or from 1 to 5. In some embodiments, m is 4, 5, 6, 7, 8, 9 or 10. In some embodiments, m is 3, 4, or 5. In some embodiments, m is 5, 6, 7, or 8.

In some embodiments, M is —OC(O)—, —C(O)O—, —N(R⁷)C(O)—, —C(O)N(R⁷)—, —C(O—R₁₃)—O—, —C(O)O(CH₂)ᵣ—, —C(O)N(R⁷) (CH₂)ᵣ—, or —C(O—R₁₃)—O—(CH₂)ᵣ—, wherein each R⁷ is independently H, alkyl, alkenyl, cycloalkyl, hydroxyalkyl, or aminoalkyl; R₁₃ is branched or unbranched C₃-C₁₀ alkyl; and r is 1, 2, 3, 4, or 5.

Another aspect of the invention relates to a lipid composition comprising a lipid compound of any formula as disclosed herein, e.g., any one of formula (I), (IA-1), (IA-2), (IIA)-(IIC), (IIA-1), (IIIA)-(IIIIE), (IIIC-1), (IVA-1)-(IVA-3), (IVC-1)-(IVC-2), and (VC-1)-(VC-6), wherein the lipid composition is a lipid nanoparticle (LNP). In some embodiments, the lipid composition further comprises a second lipid. In some embodiments, the lipid composition comprises about a 1:1 ratio of the compound and the second lipid. In some embodiments, the second lipid is cationic, anionic, ionizable, or zwitterionic lipid.

Also disclosed herein are pharmaceutical compositions comprising a pharmaceutically acceptable excipient and the lipid composition described herein, which comprises one or more lipid compounds chosen from ionizable lipids of Formula (I), (IA-1), (IA-2), (IIA)-(IIC), (IIA-1), (IIIA)-(IIIIE), (IIIC-1), (IVA-1)-(IVA-3), (IVC-1)-(IVC-2), and (VC-1)-(VC-6). The pharmaceutical compositions may further comprise a therapeutic agent. In some embodiments, the pharmaceutical compositions further comprise one or more components selected from neutral lipids, charged lipids, steroids, and polymer conjugated lipids. Such compositions may be useful for formation of lipid nanoparticles for delivery of a therapeutic agent.

Another aspect of the present disclosure provides methods for delivering a therapeutic agent to a subject (e.g., a patient) in need thereof, comprising administering to said subject (e.g., patient) the pharmaceutical composition comprises a lipid nanoparticle composition comprising a lipid compound of Formula (I), (IA-1), (IA-2), (IIA)-(IIC), (IIA-1), (IIIA)-(IIIIE), (IVA-1)-(IVA-3), (IVC-1)-(IVC-2), and (VC-1)-(VC-6), a pharmaceutically acceptable salt thereof, and/or a stereoisomer of any of the foregoing, and the therapeutic agent. In some embodiments, the method further comprises preparing a lipid nanoparticle composition comprising a lipid compound of Formula (I), (IA-1), (IA-2), (IIA)-(IIC), (IIA-1), and (IIIA)-(IIIIC), (IVA-1)-(IVA-3), (IVC-1)-(IVC-2), and (VC-1)-(VC-6), a pharmaceutically acceptable salt thereof, and/or a stereoisomer of any of the foregoing, and a therapeutic agent.

Another aspect of the present disclosure provides for extrahepatic delivery of a therapeutic agent (e.g., to the pancreas, spleen, or the lung) to a subject, comprising administering to said subject the pharmaceutical composition comprises a lipid nanoparticle composition comprising a lipid compound of Formula (I), (IA-1), (IA-2), (IIA)-(IIC), (IIA-1), (IIIA)-(IIIIE), (IVA-1)-(IVA-3), (IVC-1)-(IVC-2), and (VC-1)-(VC-6), a pharmaceutically acceptable salt thereof, and/or a stereoisomer of any of the foregoing, and the therapeutic agent. In some embodiments, the total therapeutic agent administered to the subject has a spleen to liver ratio of at least 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, or 10. In some embodiments, the total therapeutic agent administered to the subject has a spleen to liver ratio of at least 1. In some embodiments, the total therapeutic agent administered to the subject has spleen to liver ratio of at least 5.

These and other aspects of the disclosure will be apparent upon reference to the following detailed description.

### BRIEF DESCRIPTION OF DRAWINGS

FIG. 1 represents the spleen:liver ratio of average radiance (p/s/cm²/sr) of various exemplary lipid nanoparticle composition containing the exemplary lipid compounds (LNP 2230, LNP 2231), as compared to comparative lipid nanoparticle compositions containing C12-200 and MC3, respectively, based on the EPO levels determined by the in vivo bioluminescent imaging for each lipid nanoparticle composition, as described in Example 7.

### DETAILED DESCRIPTION OF THE INVENTION

### Definitions

As used herein, the following terms have the meanings ascribed to them unless specified otherwise.

Unless defined otherwise, all technical and scientific terms used herein have the same meaning as is commonly understood by one of skill in the art to which this disclosure belongs.

As used in the specification and claims, the singular form “a”, “an” and “the” include plural references unless the context clearly dictates otherwise.

Unless the context requires otherwise, throughout the present specification and claims, the word “comprise” and variations thereof, such as, “comprises” and “comprising” are to be construed in an open and inclusive sense, that is, as “including, but not limited to”.

The phrase “induce expression of a desired protein” refers to the ability of a nucleic acid to increase expression of the desired protein. To examine the extent of protein expression, a test sample (e.g., a sample of cells in culture expressing the desired protein) or a test mammal (e.g., a mammal such as a human or an animal) model such as a rodent (e.g., mouse) or a non-human primate (e.g., monkey) model is contacted with a nucleic acid (e.g., nucleic acid in combination with a lipid of the present disclosure). Expression of the desired protein in the test sample or test animal is compared to expression of the desired protein in a control sample (e.g., a sample of cells in culture expressing the desired protein) or a control mammal (e.g., a mammal such as a human or an animal) model such as a rodent (e.g., mouse) or non-human primate (e.g., monkey) model that is not contacted with or administered the nucleic acid. When the desired protein is present in a control sample or a control mammal, the expression of a desired protein in a control sample or a control mammal may be assigned a value of 1.0. In some embodiments, inducing expression of a desired protein is achieved when the ratio of desired protein expression in the test sample or the test mammal to the level of desired protein expression in the control sample or the control mammal is greater than 1, for example, about 1.1, 1.5, 2.0, 5.0 or 10.0. When a desired protein is not present in a control sample or a control mammal, inducing expression of a desired protein is achieved when any measurable level of the desired protein in the test sample or the test mammal is detected. One of ordinary skill in the art will understand appropriate assays to determine the level of protein expression in a sample, for example dot blots, northern blots, in situ hybridization, ELISA, immunoprecipitation, enzyme function, and phenotypic assays, or assays based on reporter proteins that can produce fluorescence or luminescence under appropriate conditions.

The phrase “inhibiting expression of a target gene” refers to the ability of a nucleic acid to silence, reduce, or inhibit the expression of a target gene. To examine the extent of gene silencing, a test sample (e.g., a sample of cells in culture expressing the target gene) or a test mammal (e.g., a mammal such as a human or an animal) model such as a rodent (e.g., mouse) or a non-human primate (e.g., monkey) model is contacted with a nucleic acid that silences, reduces, or inhibits expression of the target gene. Expression of the target gene in the test sample or test animal is compared to expression of the target gene in a control sample (e.g., a sample of cells in culture expressing the target gene) or a control mammal (e.g., a mammal such as a human or an animal) model such as a rodent (e.g., mouse) or non-human primate (e.g., monkey) model that is not contacted with or administered the nucleic acid. The expression of the target gene in a control sample or a control mammal may be assigned a value of 100%. In some embodiments, silencing, inhibition, or reduction of expression of a target gene is achieved when the level of target gene expression in the test sample or the test mammal relative to the level of target gene expression in the control sample or the control mammal is about 95%, 90%, 85%, 80%, 75%, 70%, 65%, 60%, 55%, 50%, 45%, 40%, 35%, 30%, 25%, 20%, 15%, 10%, 5%, or 0%. In other words, the nucleic acids are capable of silencing, reducing, or inhibiting the expression of a target gene by at least about 5%, 10%, 15%, 20%, 25%, 30%, 35%, 40%, 45%, 50%, 55%, 60%, 65%, 70%, 75%, 80%, 85%, 90%, 95%, or 100% in a test sample or a test mammal relative to the level of target gene expression in a control sample or a control mammal not contacted with or administered the nucleic acid. Suitable assays for determining the level of target gene expression include, without limitation, examination of protein or mRNA levels using techniques known to those of skill in the art, such as, e.g., dot blots, northern blots, in situ hybridization, ELISA, immunoprecipitation, enzyme function, as well as phenotypic assays known to those of skill in the art.

An “effective amount” or “therapeutically effective amount” of an active agent or therapeutic agent such as a therapeutic nucleic acid is an amount sufficient to produce the desired effect, e.g., an increase or inhibition of expression of a target sequence in comparison to the normal expression level detected in the absence of the nucleic acid. An increase in expression of a target sequence is achieved when any measurable level is detected in the case of an expression product that is not present in the absence of the nucleic acid. In the case where the expression product is present at some level prior to contact with the nucleic acid, an in increase in expression is achieved when the fold increase in value obtained with a nucleic acid such as mRNA relative to control is about 1.05, 1.1, 1.2, 1.3, 1.4, 1.5, 1.75, 2, 2.5, 3, 4, 5, 6, 7, 8, 9, 10, 15, 20, 25, 30, 40, 50, 75, 100, 250, 500, 750, 1000, 5000, 10000 or greater. Inhibition of expression of a target gene or target sequence is achieved when the value obtained with a nucleic acid such as antisense oligonucleotide relative to the control is about 95%, 90%, 85%, 80%, 75%, 70%, 65%, 60%, 55%, 50%, 45%, 40%, 35%, 30%, 25%, 20%), 15%), 110%), 5%), or 0%. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, fluorescence or luminescence of suitable reporter proteins, as well as phenotypic assays known to those of skill in the art.

The term “nucleic acid” as used herein refers to a polymer containing at least two deoxyribonucleotides or ribonucleotides in either single- or double-stranded form and includes DNA, RNA, and hybrids thereof. DNA may be in the form of antisense molecules, plasmid DNA, cDNA, PCR products, or vectors. RNA may be in the form of small hairpin RNA (shRNA), messenger RNA (mRNA), antisense RNA, miRNA, micRNA, multivalent RNA, dicer substrate RNA or viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, single nucleotide polymorphisms, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups.

“Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

The term “lipids” refers to a group of organic compounds that include, but are not limited to, esters of fatty acids and are generally characterized by being poorly soluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

A “steroid” is a compound comprising the following carbon skeleton:

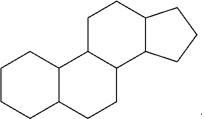 [SMILES: C1CCC2C(C1)CCC1C3CCCC3CCC21]

A non-limiting example of a steroid is cholesterol.

As used herein, the term “compound,” is meant to include all the isomers and isotopes of the structure depicted, all the pharmaceutically acceptable salts, solvates, or hydrates thereof, and all crystal forms (e.g., crystal polymorphs), crystal form mixtures, or anhydrides or hydrates thereof.

“Isotopes” refers to atoms having the same atomic number but different mass numbers resulting from a different number of neutrons in the nuclei. For example, isotopes of hydrogen include tritium (3H) and deuterium (2H).

“Isomers.” The compounds described herein or their pharmaceutically acceptable salts may include all isomers, such as geometrical isomers, optical isomers based on an asymmetrical carbon, stereoisomers, tautomers, and the like. For instance, the compounds can contain one or more stereocenters and may thus give rise to geometric isomers (e.g., double bond causing geometric E/Z isomers), enantiomers, diastereomers (e.g., enantiomers (i.e., (+) or (−)) or cis/trans isomers), and other stereoisomeric configurations that may be defined, in terms of absolute stereochemistry, as (R)- or (S)- such as for sugar anomers, or as (D)- or (L)- such as for amino acids. The present disclosure is meant to include all such possible isomers, as well as their racemic and optically pure forms. Optically active (+) and (−), (R)- and (S)-, or (D)- and (L)- isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Enantiomeric and stereometric mixtures of compounds and means of resolving them into their component enantiomers or stereoisomers are well-known. When the compounds described herein contain olefinic double bonds or other centers of geometric asymmetry, and unless specified otherwise, it is intended that the compounds include both E and Z geometric isomers. Likewise, all tautomeric forms are also intended to be included.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions. Crystallization of the compounds disclosed herein may produce a solvate.

As used herein, the term “solvate” refers to an aggregate that comprises one or more molecules of an ionizable lipid of the disclosure with one or more molecules of solvent. The solvent may be water, in which case the solvate may be a hydrate, including a monohydrate, dihydrate, hemihydrate, sesquihydrate, trihydrate, tetrahydrate and the like. Alternatively, the solvent may be an organic solvent.

As used herein, “ionizable lipid” refers to a lipid capable of being charged. In some embodiments, an ionizable lipid includes one or more positively charged amine groups. In some embodiments, ionizable lipids are ionizable such that they can exist in a positively charged or neutral form depending on pH. The ionization of an ionizable lipid affects the surface charge of a lipid nanoparticle comprising the ionizable lipid under different pH conditions. The surface charge of the lipid nanoparticle in turn can influence its plasma protein absorption, blood clearance, and tissue distribution (Semple, S. C., et al., Adv. Drug Deliv Rev 32:3-17 (1998)) as well as its ability to form endosomolytic non-bilayer structures (Hafez, I. M., et al., Gene Ther 8: 1188-1196 (2001)) that can influence the intracellular delivery of nucleic acids.

The term “polymer conjugated lipid” refers to a molecule comprising both a lipid portion and a polymer portion. A non-limiting example of a polymer conjugated lipid is a pegylated lipid. The term “pegylated lipid” refers to a molecule comprising both a lipid portion and a polyethylene glycol portion. Pegylated lipids are known in the art and include, for example, 1-(monomethoxy-polyethyleneglycol)-2,3-dimyristoylglycerol (PEG-DMG) and the like. As used herein, the terms “PEG-lipid” and “PEGylated lipid” are interchangeable and refer to a lipid comprising a polyethylene glycol component.

The term “neutral lipid” refers to any of a lipid that exists either in an uncharged or neutral zwitterionic form at a selected pH. At physiological pH, such lipids include, but are not limited to, phosphotidylcholines such as 1,2-distearoyl-sn-glycero-3-phosphocholine (DSPC), 1,2-dipalmitoyl-5n-glycero-3-phosphocholine (DPPC), 1,2-dimyristoyl-sn-glycero-3-phosphocholine (DMPC), 1-palmitoyl-2-oleoyl-sn-glycero-3-phosphocholine (POPC), 1,2-dioleoyl-sn-glycero-3-phosphocholine (DOPC), phophatidylethanolamines such as 1,2-dioleoyl-sn-glycero-3-phosphoethanolamine (DOPE), sphingomyelins (SM), ceramides, and steroids such as sterols and their derivatives. Neutral lipids may be synthetic or naturally derived.

As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

The term “liposome” as used herein refers to a composition comprising an outer lipid layer membrane (e.g., a single lipid bi-layer known as unilamellar liposomes or multiple lipid bilayers known as multilamellar liposomes) surrounding an internal aqueous space which may contain a cargo. See, e.g., Cullis et ah, Biochim. Biophys Acta, 559: 399-420 (1987), which is incorporated herein by reference in its entirety. A unilamellar liposome generally has a diameter in the range of about 20 to about 400 nanometers (nm), about 50 to about 300 nm, about 100 to about 200 nm, or about 300 to about 400 nm. A multilamellar liposome usually has a diameter in the range of about 1 to about 10 μm and may comprise anywhere from 2 to hundreds of concentric lipid bilayers alternating with layers of an aqueous phase.

The term “lipid nanoparticle” refers to a particle having at least one dimension on the order of nanometers (e.g., 1-1,000 nm) and comprising one or more compound of Formula (I). In some embodiments, lipid nanoparticles comprising one or more compounds of Formula (I), pharmaceutically acceptable salts thereof, and/or stereoisomers of any of the foregoing, are included in a composition that can be used to deliver a therapeutic agent, such as a nucleic acid (e.g., mRNA), to a target site of interest (e.g., cell, tissue, organ, tumor, and the like). In some embodiments, lipid nanoparticles comprise one or more compounds of Formula (I), pharmaceutically acceptable salts thereof, and/or stereoisomers of any of the foregoing, and a nucleic acid. In some embodiments, lipid nanoparticles comprise one or more compounds of Formula (I), pharmaceutically acceptable salts thereof, and/or stereoisomers of any of the foregoing, and a nucleic acid, and one or more other lipids selected from neutral lipids, charged lipids, steroids, and polymer conjugated lipids. In some embodiments, the therapeutic agent, such as a nucleic acid, may be encapsulated in a lipid portion of the lipid nanoparticle or an aqueous space enveloped by some or all of a lipid portion of the lipid nanoparticle, thereby protecting it from enzymatic degradation or other undesirable effects induced by the mechanisms of the host organism or cells, e.g., an adverse immune response.

In some embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In some embodiments, nucleic acids, when present in the lipid nanoparticles, are resistant in aqueous solution to degradation with a nuclease. Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos, WO 99/39741, WO 2017/117528, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548, the full disclosures of which are herein incorporated by reference in their entirety for all purposes.

As used herein, the term “size” refers to the hydrodynamic diameter of a lipid nanoparticle population. The measurement of the size of a lipid nanoformulation may be used to indicate the size and population distribution (polydispersity index, PDI) of the composition.

As used herein, the “polydispersity index” is a ratio between weight-average molar mass and Mn is the number-average molar mass that describes the homogeneity of the particle size distribution of a system. A small value, e.g., less than 0.3, indicates a narrow particle size distribution.

A polydispersity index may be used to indicate the homogeneity of a lipid composition (e.g., liposome or LNP), e.g., the particle size distribution of the liposome or LNP. A small (e.g., less than 0.3) polydispersity index generally indicates a narrow particle size distribution. A lipid composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of the lipid composition may be from about 0.10 to about 0.20.

As used herein, the term “apparent pKa” refers to the pH at which 50% of the lipid nanoformulation (e.g., LNP) is protonated. This can be used as an indicator of the pH range that the lipid nanoformulation (e.g., LNP) will be protonated, and thus initiate the endosomal escape process in a nucleotide delivery.

As used herein, the term “zeta potential” refers to the electrokinetic potential of lipid, e.g., in a lipid nanoformulation (e.g., a LNP composition). The zeta potential may describe the surface charge of a LNP composition. Zeta potential is useful in predicting organ tropism and potential interaction with serum proteins.

The zeta potential of a lipid composition (e.g., liposome or LNP) may be used to indicate the electrokinetic potential of the composition. In some embodiments, the zeta potential may describe the surface charge of a liposome or LNP. Lipid compositions (e.g., liposomes or LNP) with relatively low charges, positive or negative, are generally desirable, as more highly charged species may interact undesirably with cells, tissues, and other elements in the body. In some embodiments, the zeta potential of a liposome or LNP may be from about −10 mV to about +20 mV, from about −10 mV to about +15 mV, from about −10 mV to about +10 mV, from about −10 mV to about −+5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

As used herein, “encapsulated” by a lipid refers a therapeutic agent, such as a nucleic acid (e.g., mRNA), that is fully or partially encapsulated to by lipid nanoparticle. In some embodiments, nucleic acid (e.g., mRNA) is fully encapsulated in a lipid nanoparticle.

As used herein, “encapsulation efficiency” or “entrapment efficiency” refers to the percentage of an encapsulated cargo (e.g., a therapeutic and/or prophylactic agent) that is successfully incorporated into (e.g., encapsulated or otherwise associated with) the lipid composition (e.g., a LNP or liposome), relative to the initial total amount of therapeutic and/or prophylactic agent provided. For example, if 97 mg of therapeutic and/or prophylactic agent are encapsulated in a lipid composition out of a total 100 mg of therapeutic and/or prophylactic agent initially provided, the encapsulation efficiency may be given as 97%. Encapsulation efficiency can be used to indicate the efficiency of an encapsulated cargo (e.g., a nucleic acid molecule) loading into the lipid composition using a particular formulation method and formulation recipe.

The efficiency of encapsulation of a cargo such as a protein and/or nucleic acid, describes the amount of protein and/or nucleic acid that is encapsulated or otherwise associated with a lipid composition (e.g., liposome or LNP) after preparation, relative to the initial amount provided. The encapsulation efficiency is desirably high (e.g., at least 70%, 80%, 90%, 95%, close to 100%). The encapsulation efficiency may be measured, for example, by comparing the amount of protein or nucleic acid in a solution containing the liposome or LNP before and after breaking up the liposome or LNP with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the liposome or LNP described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

“Serum-stable” in relation to nucleic acid-lipid nanoparticles means that the nucleic acid is not significantly degraded after exposure to a serum or nuclease assay that would significantly degrade free DNA or RNA. Suitable assays include, for example, a standard serum assay, a DNAse assay, or an RNAse assay.

Some techniques of administration can lead to systemic delivery of certain agents but not others. “Systemic delivery” means that a useful, such as a therapeutic, amount of an agent is delivered to most parts of the body. Systemic delivery of lipid nanoparticles can be by any means known in the art including, for example, intravenous, intraarterial, subcutaneous, and intraperitoneal delivery. In some embodiments, systemic delivery of lipid nanoparticles is by intravenous delivery.

“Local delivery,” as used herein, refers to delivery of an agent directly to a target site within an organism. For example, an agent can be locally delivered by direct injection into a disease site such as a tumor, other target site such as a site of inflammation, or a target organ such as the liver, heart, pancreas, kidney, and the like. Local delivery can also include topical applications or localized injection techniques such as intramuscular, subcutaneous or intradermal injection. Local delivery does not preclude a systemic pharmacological effect.

As used herein, “methods of administration” may include both systemic delivery and local delivery. “Systemic delivery” means that a useful, such as a therapeutic, amount of an agent is delivered to most parts of the body. Systemic delivery of a liposome or LNP can be carried out by any means known in the art including, for example, intravenous, intraarterial, intramuscular, intradermal, subcutaneous, and intraperitoneal delivery. In some embodiments, systemic delivery of lipid nanoparticles is by intravenous delivery. “Local delivery,” as used herein, refers to delivery of an agent directly to a target site within an organism. For example, an agent can be locally delivered by direct injection into a disease site such as a tumor, other target site such as a site of inflammation, or a target organ such as the liver, heart, pancreas, kidney, and the like. Local delivery can also include topical applications or localized injection techniques such as intramuscular, subcutaneous or intradermal injection. Local delivery does not preclude a systemic pharmacological effect. As used herein, the term “polypeptide” or “polypeptide of interest” refers to a polymer of amino acid residues typically joined by peptide bonds that can be produced naturally (e.g., isolated or purified) or synthetically.

“Nucleic acid” is meant to define an oligonucleotide or polynucleotide sequence. Non-limiting examples of oligonucleotide or polynucleotides are DNA, plasmid DNA, self-amplifying RNA, mRNA, siRNA and tRNA. The term also encompasses RNA/DNA hybrids. Nucleotides are typically linked in a nucleic acid by phosphodiester bonds, although the term “nucleic acid” also encompasses nucleic acid analogs having other types of linkages or backbones (e.g., phosphoramide, phosphorothioate, phosphorodithioate, O-methylphosphoroamidate, morpholino, locked nucleic acid (LNA), glycerol nucleic acid (GNA), threose nucleic acid (TNA), and peptide nucleic acid (PNA) linkages or backbones, among others). The nucleic acids may be single-stranded, double-stranded, or contain portions of both single-stranded and double-stranded sequence. A nucleic acid can contain any combination of deoxyribonucleotides and ribonucleotides, as well as any combination of bases, including, for example, adenine, thymine, cytosine, guanine, uracil, and modified or non-canonical bases (including, e.g., hypoxanthine, xanthine, 7-methylguanine, 5,6-dihydrouracil, 5-methylcytosine, and 5 hydroxymethylcytosine).

As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-limiting group consisting of small interfering RNA (siRNA), asymmetrical interfering RNA (aiRNA), microRNA (miRNA), Dicer-substrate RNA (dsRNA), small hairpin RNA (shRNA), mRNA, and mixtures thereof.

“Alkyl” refers to a straight or branched hydrocarbon chain radical consisting solely of carbon and hydrogen atoms, having, for example, from one to twenty-four carbon atoms (C₁-C₂₄ alkyl), four to twenty carbon atoms (C₄-C₂₀ alkyl), six to sixteen carbon atoms (C₆-C₁₆ alkyl), six to nine carbon atoms (C₆-C₉ alkyl), one to fifteen carbon atoms (C₁-C₁₅ alkyl), one to twelve carbon atoms (C₁-C₁₂ alkyl), one to eight carbon atoms (C₁-C₈ alkyl) or one to six carbon atoms (C₁-C₆ alkyl) and which is attached to the rest of the molecule by a single bond, e.g., methyl, ethyl, n-propyl, 1-methylethyl (isopropyl), n-butyl, n-pentyl, 1,1-dimethylethyl (t-butyl), 3-methylhexyl, 2-methylhexyl, ethenyl, prop-1-enyl, but-1-enyl, pent-1-enyl, penta-1,4-dienyl, ethynyl, propynyl, butynyl, pentynyl, hexynyl, and the like. Unless stated otherwise specifically in the specification, an alkyl group is optionally substituted.

“Alkylene” or “alkylene chain” refers to a straight or branched divalent hydrocarbon chain linking the rest of the molecule to a radical group, consisting solely of carbon and hydrogen, having, for example, from one to twenty-four carbon atoms (C₁-C₂₄ alkylene), one to fifteen carbon atoms (C₁-C₁₅ alkylene), one to twelve carbon atoms (C₁-C₁₂ alkylene), one to eight carbon atoms (C₁-C₈ alkylene), one to six carbon atoms (C₁-C₆ alkylene), two to four carbon atoms (C₂-C₄ alkylene), one to two carbon atoms (C₁-C₂ alkylene), e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain.

The term “alkenyl” refers to a straight or branched hydrocarbon chain having one or more double bonds. Unless otherwise indicated, “alkenyl” generally refers to C₂—C alkenyl (e.g., C₂-C₆ alkenyl, C₂-C₄ alkenyl, or C₂-C₃ alkenyl). Examples of a typical alkenyl include, but not limited to, allyl, propenyl, 2-butenyl, 3-hexenyl and 3-octenyl groups.

The term “alkynyl” refers to a straight or branched hydrocarbon chain containing 2-8 carbon atoms and characterized in having one or more triple bonds. Unless otherwise indicated, “alkynyl” generally refers to C₂-C₈ alkynyl (e.g., C₂-C₆ alkynyl, C₂-C₄ alkynyl, or C₂-C₃ alkynyl). Some examples of a typical alkynyl are ethynyl, 2-propynyl, and 3-methylbutynyl, and propargyl. The sp² and sp³ carbons may optionally serve as the point of attachment of the alkenyl and alkynyl groups, respectively.

The term “cycloalkyl” or “cyclyl” as employed herein includes saturated and partially unsaturated, but not aromatic, cyclic hydrocarbon groups having 3 to 12 carbons, for example, 3 to 8 carbons, and, for example, 3 to 6 carbons, wherein the cycloalkyl group additionally may be optionally substituted. Cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” or “heteroar-” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. The term also include groups in which a heteroaromatic ring is fused to one or more aryl, cycloalkyl, or heterocyclyl rings, where the radical or point of attachment is on the heteroaromatic ring. Examples of heteroaryl groups include pyrrolyl, pyridyl, pyridazinyl, pyrazolyl, triazolyl, tetrazolyl, oxazolyl, isoxazolyl, oxadiazolyl, furanyl, imidazolyl, benzimidazolyl, pyrimidinyl, pyrazinyl, indolizinyl, thiophenyl or thienyl, quinolinyl, indolyl, thiazolyl, isothiazolyl, thiadiazolyl, purinyl, naphthyridinyl, pteridinyl, isoindolyl, benzothienyl, benzofuranyl, dibenzofuranyl, indazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(41-H)-one and the like.

The term “heterocyclyl,” “heterocycle,” “heterocyclic radical,” or “heterocyclic ring” refers to a 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. As used herein it can generally include both nonaromatic or aromatic ring (e.g., generally covered by heteroaryl). The term also include groups in which a heterocycle ring is fused to one or more aryl, cycloalkyl, or heterocyclyl rings. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

Examples of heterocyclyl groups include trizolyl, tetrazolyl, piperazinyl, pyrrolidinyl, dioxanyl, dioxolanyl, diazepinyl, oxazepinyl, thiazepinyl, morpholinyl, tetrahydrofuranyl, tetrahydropyranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, quinuclidinyl, and the like.

Examples of heterocyclyl groups also include those typical heteroaryl groups such as pyrrolyl, pyridyl, pyridazinyl, pyrazolyl, triazolyl, tetrazolyl, oxazolyl, isoxazolyl, oxadiazolyl, furanyl, imidazolyl, benzimidazolyl, pyrimidinyl, pyrazinyl, indolizinyl, thiophenyl or thienyl, quinolinyl, indolyl, thiazolyl, isothiazolyl, thiadiazolyl, purinyl, naphthyridinyl, pteridinyl, isoindolyl, benzothienyl, benzofuranyl, dibenzofuranyl, indazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one and the like.

A divalent radical of an alkyl, alkenyl, aryl, heteroaryl, cycloalkyl, heterocyclyl is formed by removal of a hydrogen atom from an alkyl, alkenyl, aryl, heteroaryl, cycloalkyl, and heterocyclyl radical, respectively (or by removal of two hydrogen atoms from an alkane, alkene, arene, heteroarene, cycloalkane, or heterocycle, respectively).

For instance, the term “bivalent heterocycle” or “divalent heterocyle” refers to a divalent form of a heterocycle, i.e., a bivalent or divalent radical that is formed by removal of a hydrogen atom from a heterocyclyl radical (or by removal of two hydrogen atoms from a heterocycle). For instance, a bivalent or divalent form of a heterocycle is formed by removal of a hydrogen atom from each of two different atoms of the heterocycle ring. By way of an example, a bivalent or divalent form of a 1,2,3, triazole

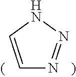

is formed by removal of a hydrogen atom from each of two different atoms of the triazole ring (from the carbon atom or nitrogen atom), and may have a structure of

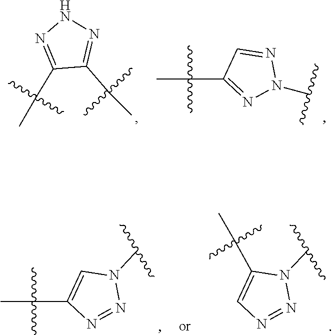 [SMILES: *c1n[nH]nc1* ; *c1cnn(*)n1 ; *c1cn(*)nn1 ; *c1cnnn1*]

The term “alkoxy” refers to an —O-alkyl radical.

The term “aminoalkyl” refers to an alkyl substituted with an amino. The term “alkylamino” refers to an amino substituted with an alkyl.

The term “aminocarbonyl” refers to an —C(O)-amino radical.

The term “substituted” used herein means any of the above groups (e.g., alkyl, hydroxyalkyl, alkylene, cycloalkyl, cycloalkylene, amino, aminocarbonyl, heterocyclyl, or heteroaryl) wherein one or more hydrogen atoms is replaced by a bond to a non-hydrogen atom such as, but not limited to: a halogen atom such as F, Cl, Br, or I; oxo groups (=O); hydroxyl groups (—OH); alkoxy, alkoxyalkyl, aralkoxy, alkyl such as C₁-C₁₂ alkyl groups; cycloalkyl groups; alkenyl, alkynyl, aryl, aralkyl heterocyclyl, heterocyclyl, heteroaryl, thiol, alkylthio, arylthio, alkylthioalkyl, arylthioalkyl, alkylsulfonyl, alkylsulfonylalkyl, arylsulfonylalkyl, aryloxy, carboxyalkyl, alkoxycarbonylalkyl, aminocarbonylalkyl, acyl, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, aralkoxycarbonyl, sulfonyl, alkylaminolactams, alkylaminoheteroaryls, alkylaminoheterocycyls, and aminosulfonamides. Exemplary substituents also include: —(C(═)OR; —O(C═O)R; —C(═O)R; —OR; —S(O)ₓR; —S—SR; —C(═C))SR; —SC(═O)R; —NRR′; —R′C(═O)R; —C(═O)RR′; —RC(═O)R′R″; —OC(═O)RR′; —RC(═O)OR′; —R′S(O)ₓR″R; —R′S(O)ₓR; and —S(O)ₓRR′, wherein: R, R′, and R″ is, at each occurrence, independently H, C₁-C₁₅ alkyl or cycloalkyl, heterocyclyl, or hereoaryl that can be optionally substituted, and x is 0, 1 or 2. In some embodiments, the substituent is a C₁-C₁₂ alkyl group. In some embodiments, the substituent is a cycloalkyl group. In some embodiments, the substituent is a halo group, such as fluoro. In some embodiments, the substituent is an oxo group. In some embodiments, the substituent is a hydroxyl group. In some embodiments, the substituent is a hydroxyalkylene group (—R—OH). In some embodiments, the substituent is an alkoxy group (—OR). In some embodiments, the substituent is a carboxyl group. In some embodiments, the substituent is an amino group (—NRR′). Suitable substituents also include divalent substituents on a saturated carbon atom, including but are not limited to: ═O, ═S, ═NNR*2, ═NNHC(O)R*, ═NNHC(O)OR*, ═NNHS(O)2R*, ═NR*, ═NOR*, —O(C(R*2))2-3O—, or —S(C(R*2))2-3S—, wherein each independent occurrence of R* is selected from hydrogen, substituted or unsubstituted C₁₋₆ alkyl, or an unsubstituted 5-6-membered saturated or partially unsaturated ring, or an aryl ring having 0-4 heteroatoms independently selected from nitrogen, oxygen, or sulfur.

“Halo” or “halogen” refers to any radical of fluorine, chlorine, bromine or iodine.

“Optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

The present disclosure is also meant to encompass all pharmaceutically acceptable compounds of all the Formulas identified herein being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I respectively. These isotopically-labelled compounds could be useful to help determine or measure the effectiveness of the compounds, by characterizing, for example, the site or mode of action, or binding affinity to pharmacologically important site of action. Certain isotopically-labelled compounds of structure (I), (IA) or (IB), for example, those incorporating a radioactive isotope, are useful in drug and/or substrate tissue distribution studies. The radioactive isotopes tritium, i.e., ³H, and carbon-14, i.e., ¹⁴C, may be useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be useful in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula (I) can generally be prepared by conventional techniques known to those skilled in the art or by processes analogous to those described in the Preparations and Examples as set out below using an appropriate isotopically-labeled reagent in place of the non-labeled reagent previously employed.

The present disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, embodiments of the disclosure include compounds produced by a process comprising administering an ionizable lipid of this disclosure to a mammal for a period of time sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts, “Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, l-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, sodium, potassium, lithium, ammonium, calcium, magnesium, iron, zinc, copper, manganese, aluminum salts and the like. Non-limiting examples of inorganic salts are ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Non-limiting examples of organic bases are isopropylamine, diethylamine, ethanolamine, trimethylamine, dicyclohexylamine, choline and caffeine.

Crystallization of ionizable lipid(s) disclosed herein may produce a solvate. As used herein, the term “solvate” refers to an aggregate that comprises one or more molecules of an ionizable lipid of the disclosure with one or more molecules of solvent. The solvent may be water, in which case the solvate may be a hydrate. Alternatively, the solvent may be an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. Solvates of compound of the disclosure may be true solvates, while in other cases, the compound of the disclosure may merely retain adventitious water or be a mixture of water plus some adventitious solvent.

A “pharmaceutical composition” refers to a composition which may comprise an ionizable lipid of the disclosure and a medium generally accepted in the art for the delivery of the biologically active compound to mammals, e.g., humans. Such a medium includes pharmaceutically acceptable carriers, diluents or excipients therefor.

“Effective amount” or “therapeutically effective amount” refers to that amount of an ionizable lipid of the disclosure which, when administered to a mammal, such as a human, is sufficient to effect treatment in the mammal, such as a human. The amount of a lipid nanoparticle of the disclosure which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, such as a human, having the disease or condition of interest, and includes:
(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; or
(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the disclosure, or their pharmaceutically acceptable salts may contain one or more stereocenters and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)- isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

In the following description, certain specific details are set forth to provide a thorough understanding of various embodiments of the disclosure. However, one of ordinary skill in the art will understand that the disclosure may be practiced without these details.

### Ionizable Lipid Compounds

One aspect of the invention relates to a compound of Formula (I):

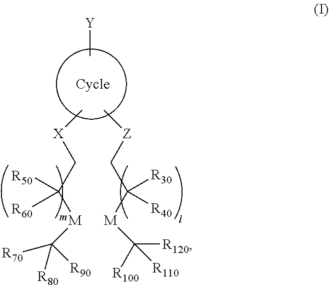

a pharmaceutically acceptable salt thereof, or a stereoisomer of any of the foregoing, wherein:

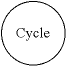

is cyclic or heterocyclic moiety;
Y is alkyl, hydroxy, hydroxyalkyl or

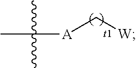

A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
each of X and Z is independently absent, —O—, —CO—, —N(R⁷)—, —O-alkylene-; -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, hydroxyalkyl, or aminoalkyl;
each M is independently a biodegradable moiety;
each of R₃₀, R₄₀, R₅₀, R₆₀, R₇₀, R₈₀, R₉₀, R₁₀₀, R₁₁₀, and R₁₂₀ is independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
each of l and m is an integer from 1 to 10;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl.

In some embodiments, disclosed are ionizable lipids of Formula (I):

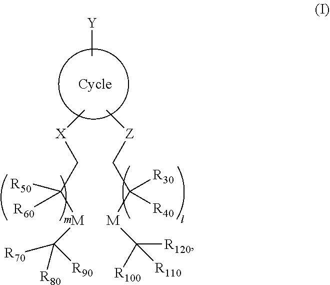

a pharmaceutically acceptable salt thereof, or a stereoisomer of any of the foregoing, wherein:

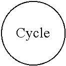

is cyclic or heterocyclic moiety;
Y is alkyl, hydroxy, hydroxyalkyl, or

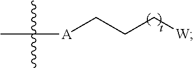

A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—;
each of X and Z is independently absent, —O—, —N(R⁷)—, —O-alkylene-; -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, hydroxyalkyl, or aminoalkyl;
each M is independently a biodegradable moiety;
each of R₃₀, R₄₀, R₅₀, R₆₀, R₇₀, R₈₀, R₉₀, R₁₀₀, R₁₁₀, and R₁₂₀ is independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
each of l and m is an integer from 1 to 10;
t is 0, 1, 2, or 3; and
W is hydroxyl, hydroxyalkyl, or one of the following moieties:

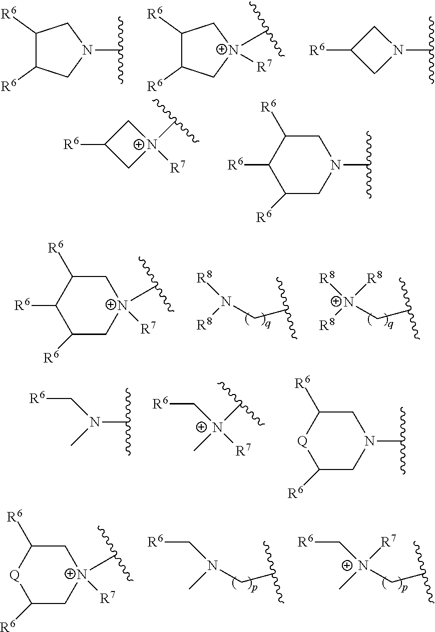

wherein
   Q is —O— or —N(R⁷);
   R⁶ is independently H, alkyl, hydroxyalkyl, amino, aminoalkyl, thiol, or thiolalkyl;
   each R⁸ is independently H, alkyl, hydroxyalkyl, amino, aminoalkyl, thiol, or thiolalkyl, or two R⁸ may form a ring;
   q is 0, 1, 2, 3, 4, or 5; and
   p is 0, 1, 2, 3, 4, or 5.

In some embodiments, in any of the formulas described herein, Y is hydroxyl,

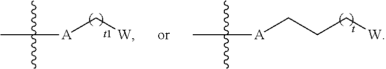

In some embodiments, each of R₇₀ and R₈₀ is H; and R₉₀ is C₁-C₁₅ branched or unbranched alkyl, C₁-C₁₅ branched or unbranched alkenyl, cycloalkyl or substituted cycloalkyl. In some embodiments, R₉₀ is C₁-C₁₅ branched or unbranched alkyl, C₁-C₁₅ branched or unbranched alkenyl. In some embodiments, R₉₀ is C₁-C₁₅ branched or unbranched alkyl. In some embodiments R₉₀ is C₁-C₁₂ branched or unbranched alkyl.

In some embodiments, R₇₀ is H; and each of R₈₀ and R₉₀ is independently C₁-C₁₅ branched or unbranched alkyl, C₁-C₁₅ branched or unbranched alkenyl, or cycloalkyl or substituted cycloalkyl. In some embodiments, each of R₈₀ and R₉₀ is independently C₁-C₁₅ branched or unbranched alkyl, C₁-C₁₅ branched or unbranched alkenyl. In some embodiments, each of R₈₀ and R₉₀ is independently C₁-C₁₅ branched or unbranched alkyl. In some embodiments, each of R₈₀ and R₉₀ is independently C₁-C₁₂ branched or unbranched alkyl. In some embodiments, each of R₈₀ and R₉₀ is independently C₁-C₈ branched or unbranched alkyl.

In some embodiments, R₁₀₀ is H; and each of R₁₁₀ and R₁₂₀ is independently C₁-C₁₅ branched or unbranched alkyl, C₁-C₁₅ branched or unbranched alkenyl, or cycloalkyl or substituted cycloalkyl. In some embodiments, each of R₁₁₀ and R₁₂₀ is independently C₁-C₁₅ branched or unbranched alkyl, C₁-C₁₅ branched or unbranched alkenyl. In some embodiments, each of R₁₁₀ and R₁₂₀ is independently C₁-C₁₅ branched or unbranched alkyl. In some embodiments, each of R₁₁₀ and R₁₂₀ is independently C₁-C₁₂ branched or unbranched alkyl. In some embodiments, each of R₁₁₀ and R₁₂₀ is independently C₁-C₈ branched or unbranched alkyl.

In some embodiments, disclosed are ionizable lipids of Formula (IA-1) or (IA-2):

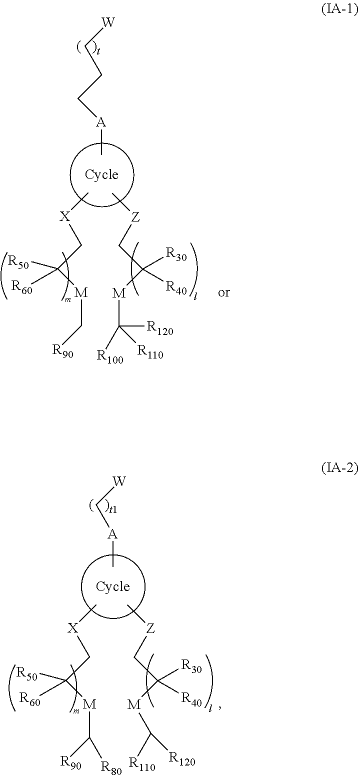

a pharmaceutically acceptable salt thereof, or a stereoisomer of any of the foregoing, wherein:

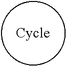

is cyclic or heterocyclic moiety;
A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
X is absent, —O—, —CO—, —N(R⁷)—, —O-alkylene-, -alkylene-O—, —OC(O)—, —C(O)O—, —N(R⁷)C(O)—, —C(O)N(R⁷)—, or —S—;
Z is absent, —O—, —N(R⁷)—, —O-alkylene-, -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, hydroxyalkyl, or aminoalkyl;
each M is independently a biodegradable moiety;
each of R₃₀, R₄₀, R₅₀, R₆₀, R₁₀₀, R₁₁₀, and R₁₂₀ is independently H, C₁-C₁₆ branched or unbranched alkyl, or C₁-C₁₆ branched or unbranched alkenyl, optionally interrupted with heteroatom or substituted with OH, SH, or halogen;
R₉₀ is C₁-C₁₅ branched or unbranched alkyl, C₁-C₁₅ branched or unbranched alkenyl, or cycloalkyl or substituted cycloalkyl;
t is 0, 1, 2, or 3;
t1 is an integer from 0 to 10;
l is an integer from 1 to 10;
m is an integer from 1 to 10; and
W is hydroxyl, hydroxyalkyl, or one of the following moieties:

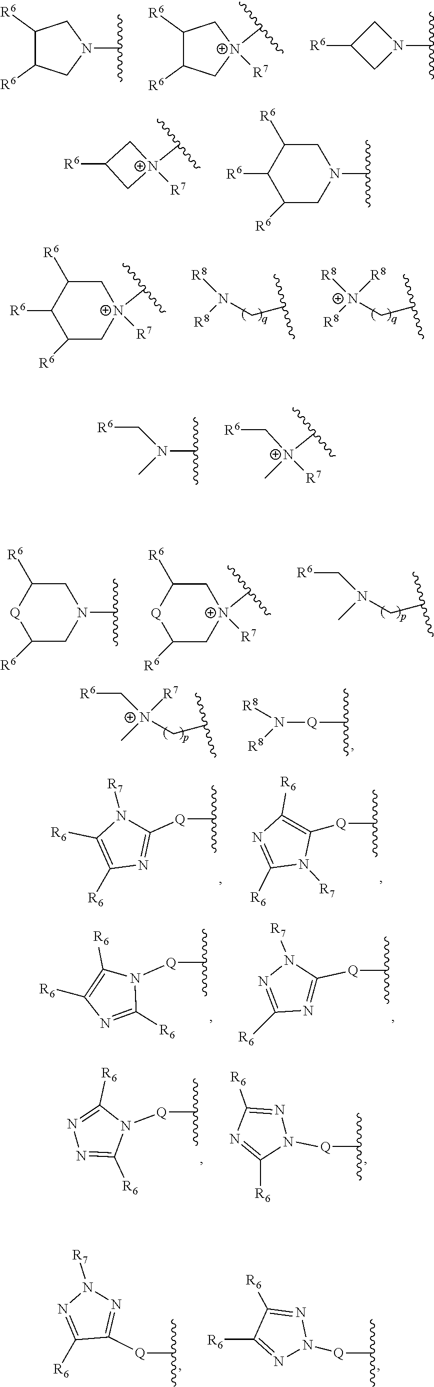

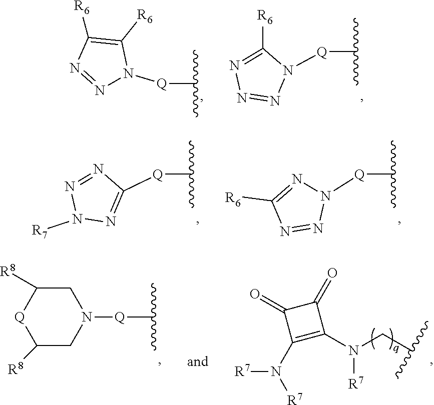

each Q is independently absent, —O—, —C(O)—, —C(S)—, —C(O)O—, —C(R⁷)₂—, —C(O)N(R⁷)—, —C(S)N(R⁷)—, or —N(R⁷);
each R⁶ is independently H, alkyl, hydroxyl, hydroxyalkyl, alkoxy, amino, aminoalkyl, alkylamino, thiol, thiolalkyl, or N⁺(R⁷)₃-alkylene-Q-;
each R⁸ is independently H, alkyl, hydroxyalkyl, amino, aminoalkyl, thiol, or thiolalkyl, or two R⁸ together with the nitrogen atom may form a ring;
each q is independently 0, 1, 2, 3, 4, or 5; and
each p is independently 0, 1, 2, 3, 4, or 5.

Embodiments regarding various variables in the formulas (I) and (IA-1) and (IA-2) are further discussed below.

In some embodiments, in any of the formulas described herein,

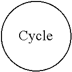

has a structure of formula

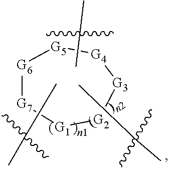

wherein:
each of G₁, G₂, G₃, G₄, G₅, and G₆, is independently C(R′)(R″), O, or N, provided that no more than two of G₁-G₆ are O or N;
R′ and R″ are each independently absent, H, alkyl, or two R′ from the two neighboring G together form a second 5- to 7-membered cyclic or heterocylic ring; and
n₁ and n₂ are each independently 0 or 1.

In some embodiments, in any of the formulas described herein,

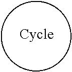

is selected from pyrrolidine, piperidine, piperazine, cyclohexane, cyclopentane, tetrahydrofuran, tetrahydropyran, morpholine, and dioxane.

In any of the formulas described herein, in some embodiments,

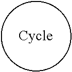

is a 5- to 7-membered, monocyclic ring. In some embodiments,

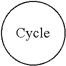

is a 5- to 7-membered, monocyclic, cycloalkane ring. In some embodiments,

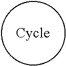

is a 5- to 7-membered, monocyclic, heterocycle ring.

In any of the formulas described herein, in some embodiments,

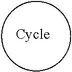

is a bicyclic or tricyclic ring, i.e., containing two or more rings, such as fused rings.

In any of the formulas described herein, in some embodiments,

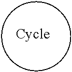

is selected from the group consisting of

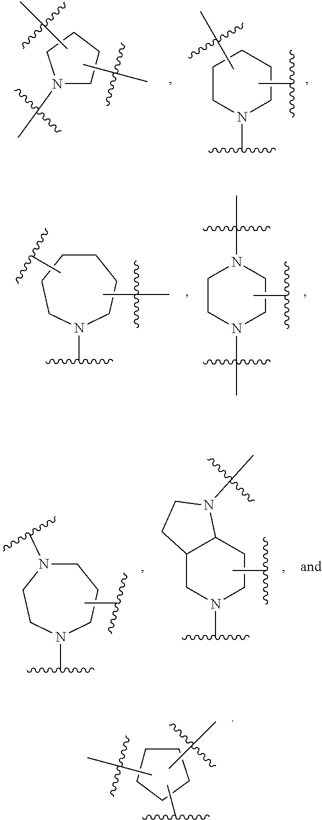

In some embodiments,

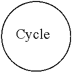

is selected from the group consisting of

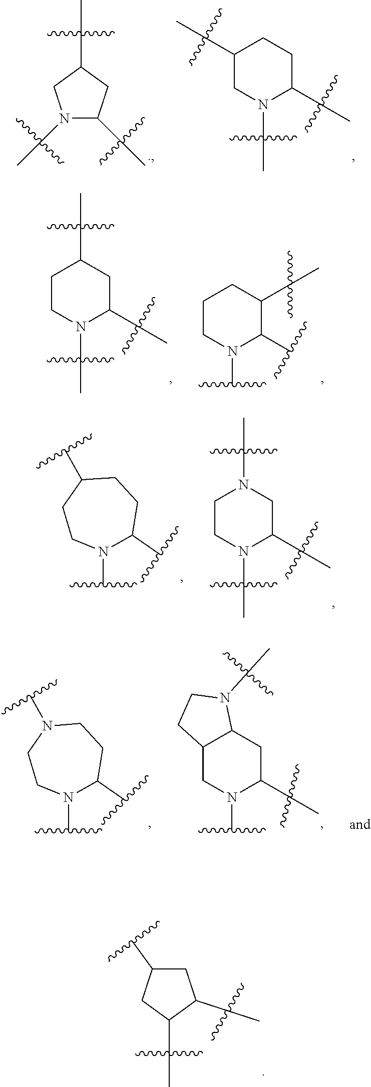

In some embodiments

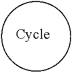

has a structure of

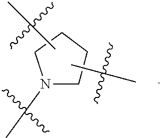

In one embodiment

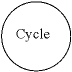

has a structure of

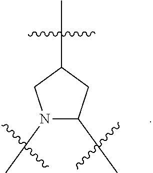 [SMILES: *C1CC(*)N(*)C1]

In some embodiments,

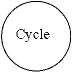

has a structure of

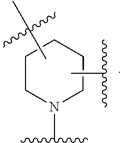

In one embodiment,

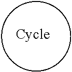

has a structure of

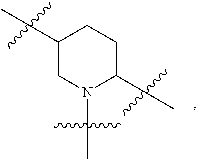 [SMILES: *C1CCC(*)N(*)C1]

In one embodiment,

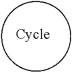

has a structure of

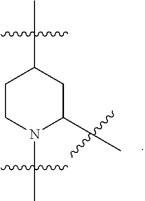 [SMILES: *C1CCN(*)C(*)C1]

In one embodiment,

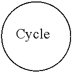

has a structure of

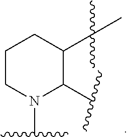 [SMILES: *C1CCCN(*)C1*]

In some embodiments,

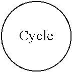

has a structure of

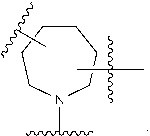

In one embodiment

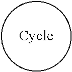

has a structure of

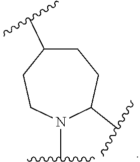 [SMILES: *C1CCC(*)N(*)CC1]

In some embodiments,

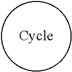

has a structure of

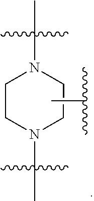

In one embodiment,

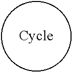

has a structure of

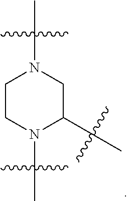 [SMILES: *C1CN(*)CCN1*]

In some embodiments,

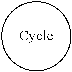

has a structure of

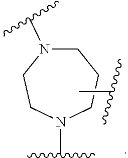

In one embodiment,

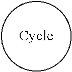

has a structure of

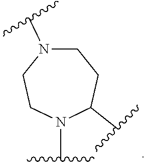 [SMILES: *C1CCN(*)CCN1*]

In some embodiments,

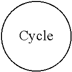

has a structure of

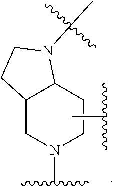

In one embodiment,

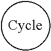

has a structure of

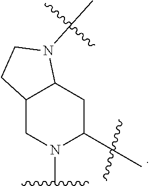 [SMILES: *C1CC2C(CCN2*)CN1*]

In some embodiments,

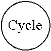

has a structure of

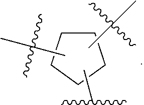

In one embodiment,

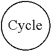

has a structure of

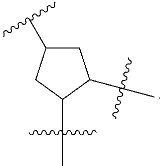 [SMILES: *C1CC(*)C(*)C1]

In some embodiments, in any of the formulas described herein, for Y,

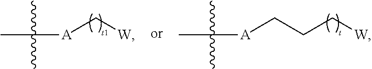

A is absent, —O—, —N(R⁷)—, N(R⁷)C(O)—,

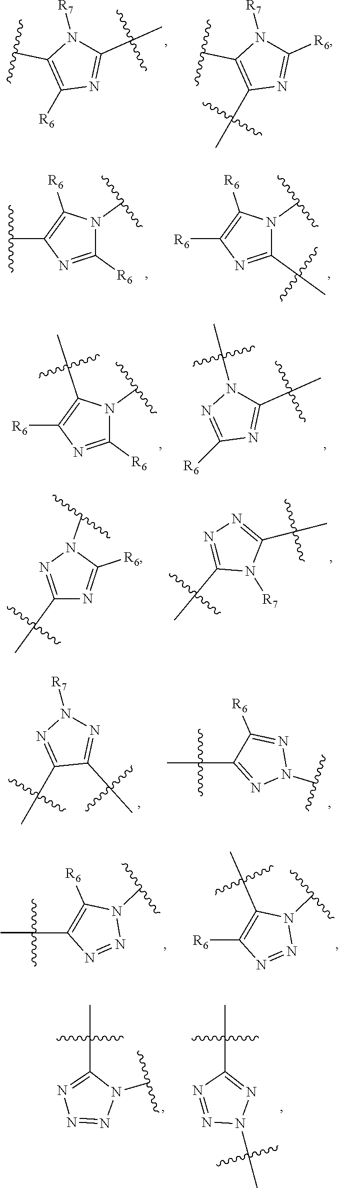

—OC(O)—, or —C(O)O—, wherein R⁶ is independently H, alkyl, hydroxyl, hydroxyalkyl, amino, aminoalkyl, thiol, thiolalkyl, or N⁺(R⁷)₃-alkylene-Q-; and R⁷ is H or C₁-C₃ alkyl. In one embodiment, A is absent. In one embodiment, A is —O—. In one embodiment, A is —N(R⁷)—, wherein R⁷ is H or C₁-C₃ alkyl. In one embodiment, A is —OC(O)— or —C(O)O—. In one embodiment, A is

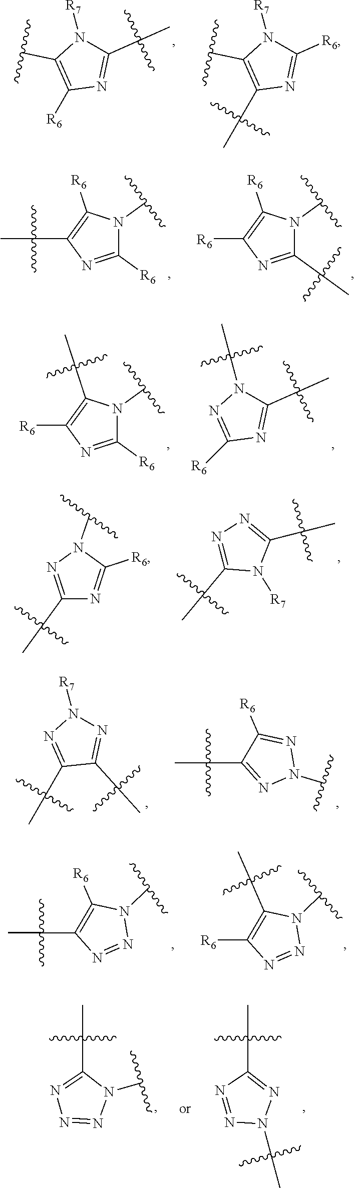

In some embodiments, in any of the formulas described herein, t1 is 0, 1, 2, 3 or 4; and t is 0, 1, or 2.

In some embodiments, in any of the formulas described herein, W is hydroxyl, hydroxyalkyl, or one of the following moieties:

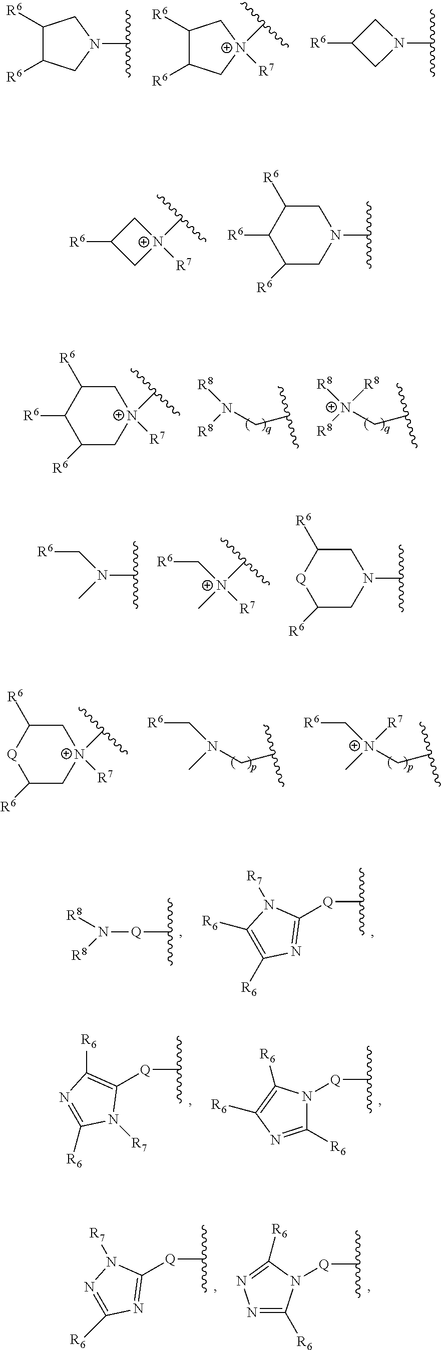

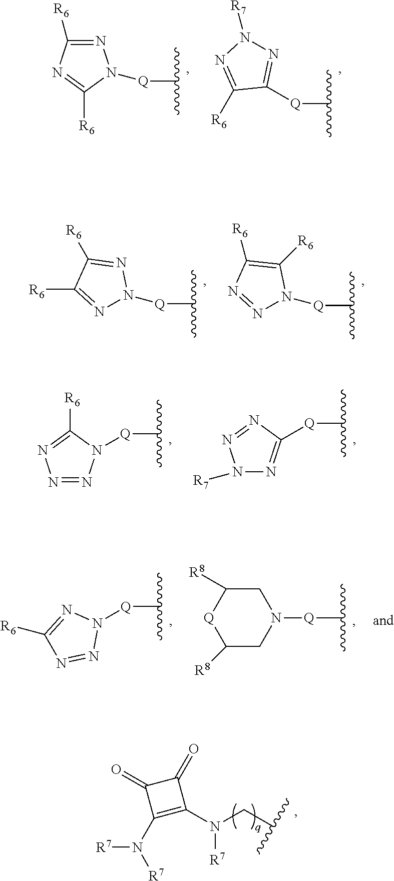

wherein:
each Q is independently absent, —O—, —C(O)—, —C(S)—, —C(O)O—, —C(R⁷)₂—, —C(O)N(R⁷)—, —C(S)N(R⁷)—, or —N(R⁷)—;
each R⁶ is independently H, alkyl, hydroxyl, hydroxyalkyl, alkoxy, amino, aminoalkyl, alkylamino, thiol, thiolalkyl, or N⁺(R⁷)₃-alkylene-Q-;
each R⁸ is independently H, alkyl, hydroxyalkyl, amino, aminoalkyl, thiol, or thiolalkyl, or two R⁸ together with the nitrogen atom may form a ring;
each q is independently 0, 1, 2, 3, 4, or 5; and
each p is independently 0, 1, 2, 3, 4, or 5.

In some embodiments, in any of the formulas described herein, W is OH,

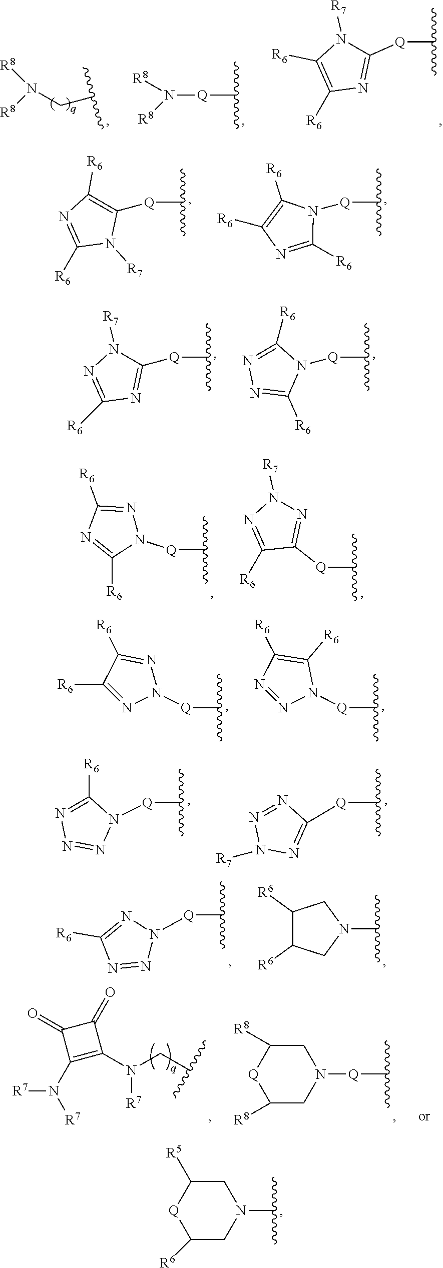

wherein:
q is 0,
each R⁸ is independently H, C₁-C₃ alkyl, or hydroxyalkyl, or two R⁸ together with the nitrogen atom form a 5-membered ring optionally substituted with one or more alkyl groups,
each R⁶ is independently H, hydroxyl, C₁-C₃ alkyl, or -Q-alkylene-N⁺(R⁷)₃,
each Q is independently absent, —O—, —C(O)—, —N(R⁷)—, —C(R⁷)₂—, —C(O)O—, —C(O)N(R⁷)—, or —C(S)N(R⁷)—, and
each R⁷ is independently H, C₁-C₃ alkyl, or hydroxyalkyl.

In some embodiments, in any of the formulas described herein, W is OH,

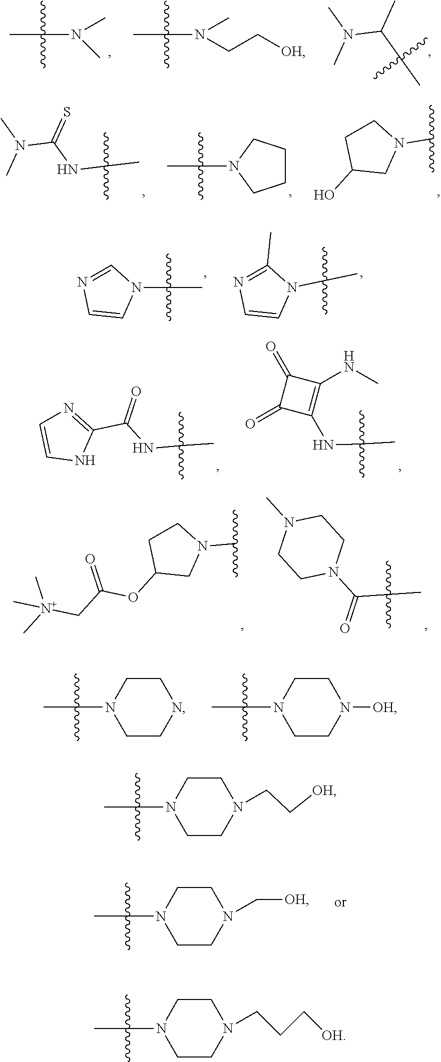

In some embodiments, W is OH.

In some embodiments, W is

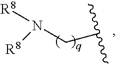

wherein q is 0 and each R⁸ is independently H, C₁-C₃ alkyl, or hydroxyalkyl. In one embodiment, W is

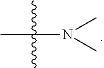 [SMILES: *N(C)C]

In one embodiment, W is

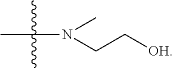 [SMILES: *N(C)CCO]

In some embodiments, W is

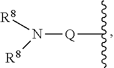

wherein each R⁸ is independently H, C₁-C₃ alkyl, or hydroxyalkyl, Q is —N(R⁷)—, —C(R⁷)₂—, —C(O)O—, —C(O)N(R⁷)—, or —C(S)N(R^{7′}) and each R⁷ is independently H, C₁-C₃ alkyl, or hydroxyalkyl. In one embodiment, W is

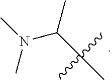 [SMILES: *C(C)N(C)C]

In one embodiment, W is

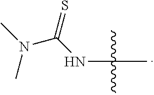 [SMILES: *NC(=S)N(C)C]

In some embodiments, W is

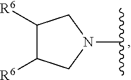

wherein each R⁶ is independently H, C₁-C₃ alkyl, hydroxyl, hydroxyalkyl, alkoxy, amino, aminoalkyl, alkylamino, C₁-C₃ alkyl, or -Q-alkylene-N⁺(R⁷)₃. In one embodiment, W is

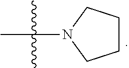 [SMILES: *N1CCCC1]

In one embodiment, W is

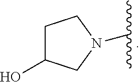 [SMILES: *N1CCC(O)C1]

In one embodiment, W is

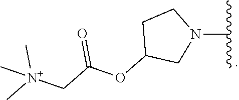 [SMILES: *N1CCC(OC(=O)C[N+](C)(C)C)C1]

In some embodiments, W is

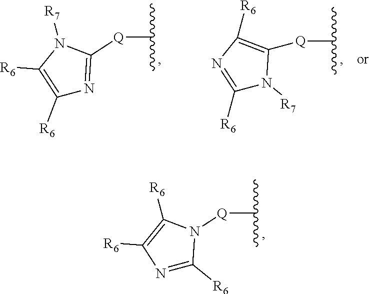

wherein each Q is independently absent, —N(R⁷)—, —C(R⁷)₂—, —C(O)O—, —C(O)N(R⁷)—, or —C(S)N(R⁷)—; each R⁶ is independently H, alkyl, hydroxyl, hydroxyalkyl, alkoxy, amino, aminoalkyl, alkylamino, thiol, thiolalkyl, or N(R⁷)₃-alkylene-Q-; and each R⁷ is independently H, C₁-C₃ alkyl, hydroxy, or hydroxyalkyl. In one embodiment, W is

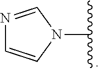 [SMILES: *n1ccnc1]

In one embodiment, W is

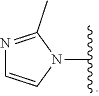 [SMILES: *n1ccnc1C]

In one embodiment, W is

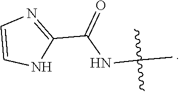 [SMILES: *NC(=O)c1ncc[nH]1]

In some embodiments, W is

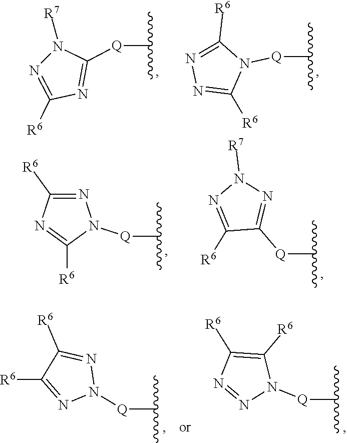

wherein each Q is independently absent, —N(R⁷)—, —C(R⁷)₂—, —C(O)O—, —C(O)N(R⁷)—, or —C(S)N(R⁷)—; each R⁶ is independently H, alkyl, hydroxyl, hydroxyalkyl, alkoxy, amino, aminoalkyl, alkylamino, thiol, thiolalkyl, or N⁺(R⁷)₃-alkylene-Q-; and each R⁷ is independently H, C₁-C₃ alkyl, hydroxy, or hydroxyalkyl.

In some embodiments, W is

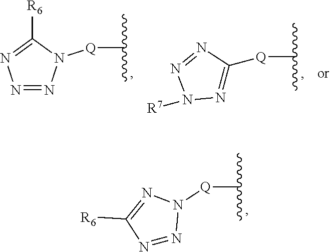

wherein each Q is independently absent, —N(R⁷)—, —C(R⁷)₂—, —C(O)O—, —C(O)N(R⁷)—, or —C(S)N(R⁷)—; each R⁶ is independently H, alkyl, hydroxyl, hydroxyalkyl, alkoxy, amino, aminoalkyl, alkylamino, thiol, thiolalkyl, or N⁺(R⁷)₃-alkylene-Q-; and each R⁷ is independently H, C₁-C₃ alkyl, hydroxy, or hydroxyalkyl.

In some embodiments,

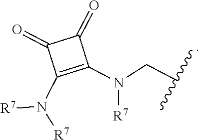

wherein q is 0 or 1, and each R⁷ is independently H, C₁-C₃ alkyl. In one embodiment, W is

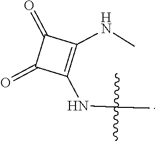 [SMILES: *Nc1c(NC)c(=O)c1=O]

In some embodiments, W is

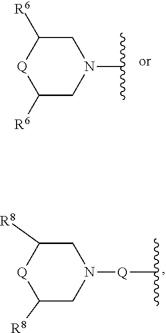

wherein each Q is independently —N(R′)—, —C(R⁷)₂—, —C(O)O—, —C(O)N(R⁷)—, or —C(S)N(R⁷)—; each R⁶ is independently H or C₁-C₃ alkyl, each Q is independently —O—, —C(O)—, —N(R⁷) and R⁷ is H, C₁-C₃ alkyl, hydroxy, or hydroxyalkyl. In one embodiment, W is

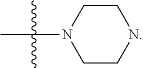

In one embodiment, W is

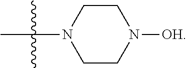 [SMILES: *N1CCN(O)CC1]

In one embodiment, W is

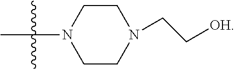 [SMILES: *N1CCN(CCO)CC1]

In one embodiment, W is

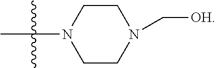 [SMILES: *N1CCN(CO)CC1]

In one embodiment, W is

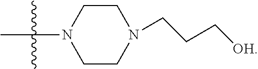 [SMILES: *N1CCN(CCCO)CC1]

In one embodiment, W is

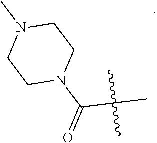 [SMILES: *C(=O)N1CCN(C)CC1]

In one embodiment, W is

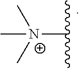 [SMILES: *[N+](C)(C)C]

In one embodiment, W is

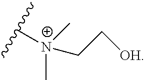 [SMILES: *[N+](C)(C)CCO]

In one embodiment, W is

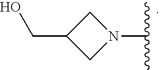 [SMILES: *N1CC(CO)C1]

In one embodiment, W is

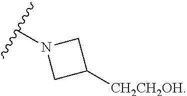

In one embodiment, W is

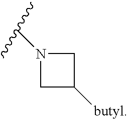

In one embodiment, W is

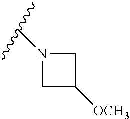 [SMILES: *N1CC(OC)C1]

In one embodiment, W is

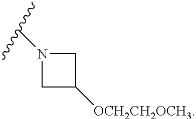

In one embodiment, W is

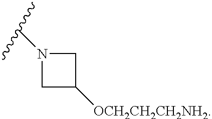

In one embodiment, W is

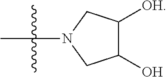 [SMILES: *N1CC(O)C(O)C1]

In one embodiment, W is

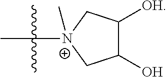 [SMILES: *[N+]1(C)CC(O)C(O)C1]

In one embodiment, W is

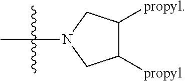

In one embodiment, W is

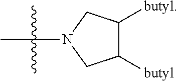

In one embodiment, W is

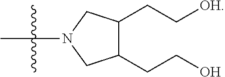 [SMILES: *N1CC(CCO)C(CCO)C1]

In one embodiment, W is

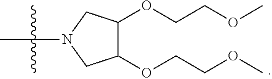 [SMILES: *N1CC(OCCOC)C(OCCOC)C1]

In one embodiment, W is

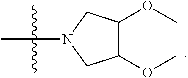 [SMILES: *N1CC(OC)C(OC)C1]

In one embodiment, W is

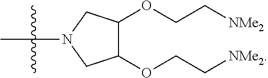 [SMILES: *N1CC(OCCN(C)C)C(OCCN(C)C)C1]

In one embodiment, W is

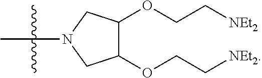

In one embodiment, W is

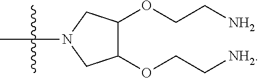 [SMILES: *N1CC(OCCN)C(OCCN)C1]

In one embodiment, W is

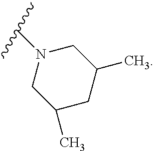 [SMILES: *N1CC(C)CC(C)C1]

In one embodiment, W is

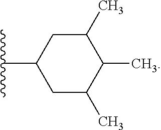 [SMILES: *C1CC(C)C(C)C(C)C1]

In one embodiment, W is

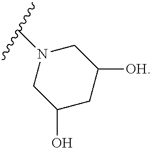 [SMILES: *N1CC(O)CC(O)C1]

In one embodiment, W is

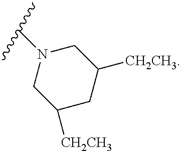 [SMILES: *N1CC(CC)CC(CC)C1]

In one embodiment, W is

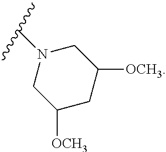 [SMILES: *N1CC(OC)CC(OC)C1]

In one embodiment, W is

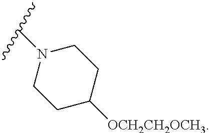

In one embodiment, W is

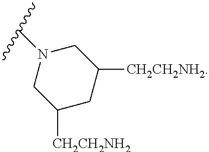

In one embodiment, W is

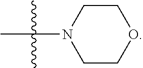 [SMILES: *N1CCOCC1]

In one embodiment, W is

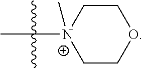 [SMILES: *[N+]1(C)CCOCC1]

In one embodiment, W is

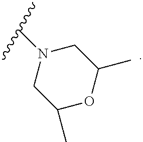 [SMILES: *N1CC(C)OC(C)C1]

In one embodiment, W is

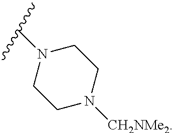

In some embodiments, in any of the formulas described herein, Y or

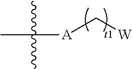

### is: OH,

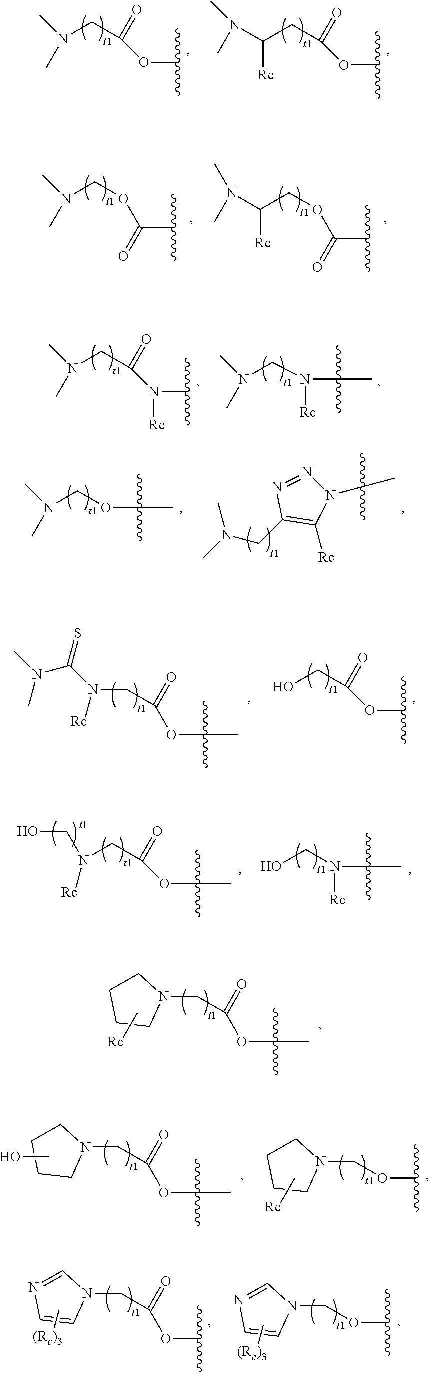

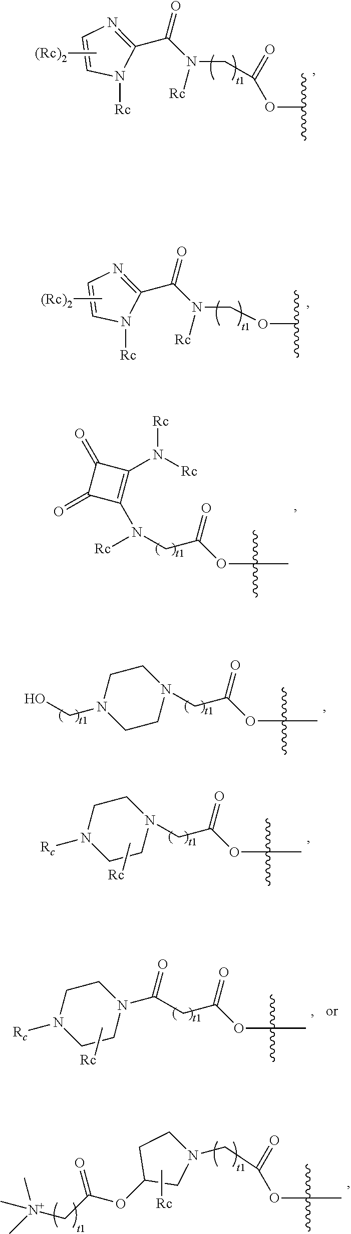

wherein each R^{c} is independently H or C₁-C₃ alkyl, and each t1 is independently 1, 2, 3, or 4.

In some embodiments, in any of the formulas described herein, Y or

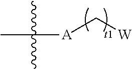

### is: OH,

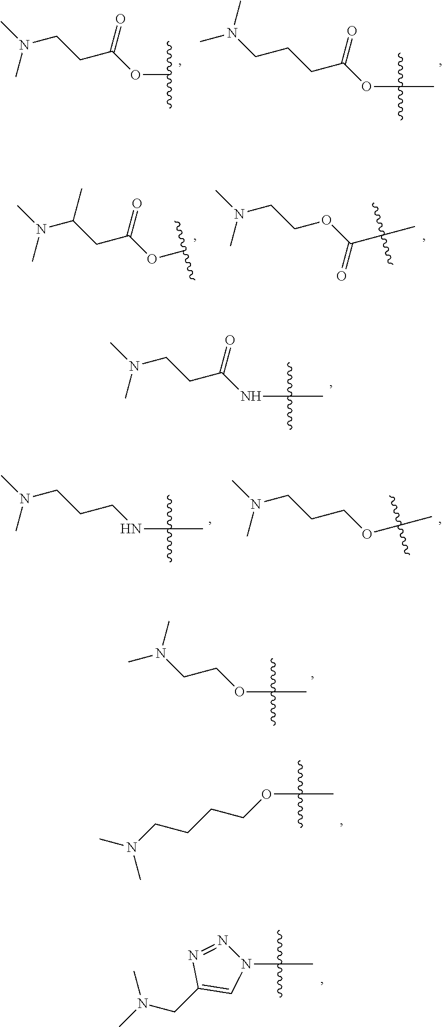

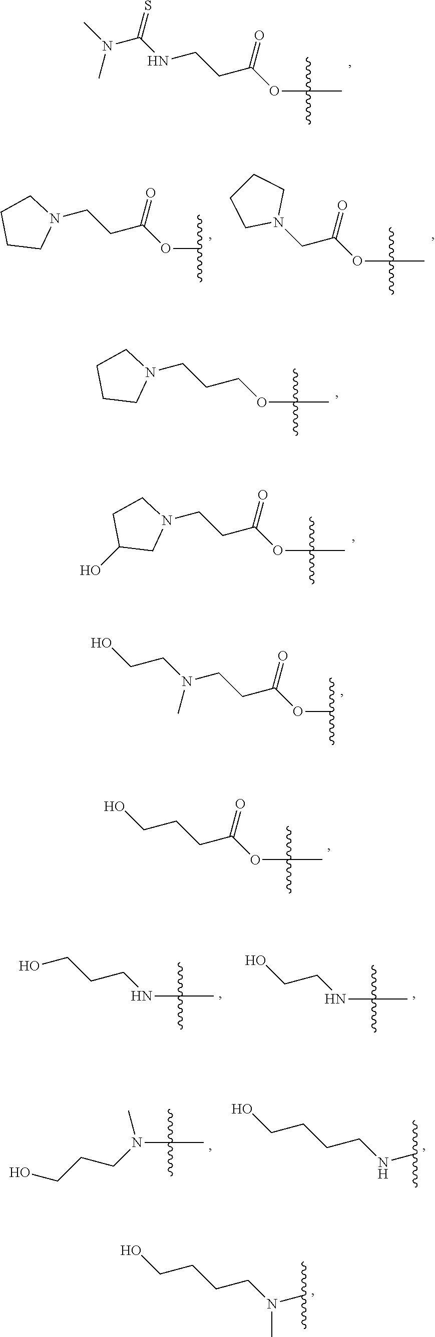

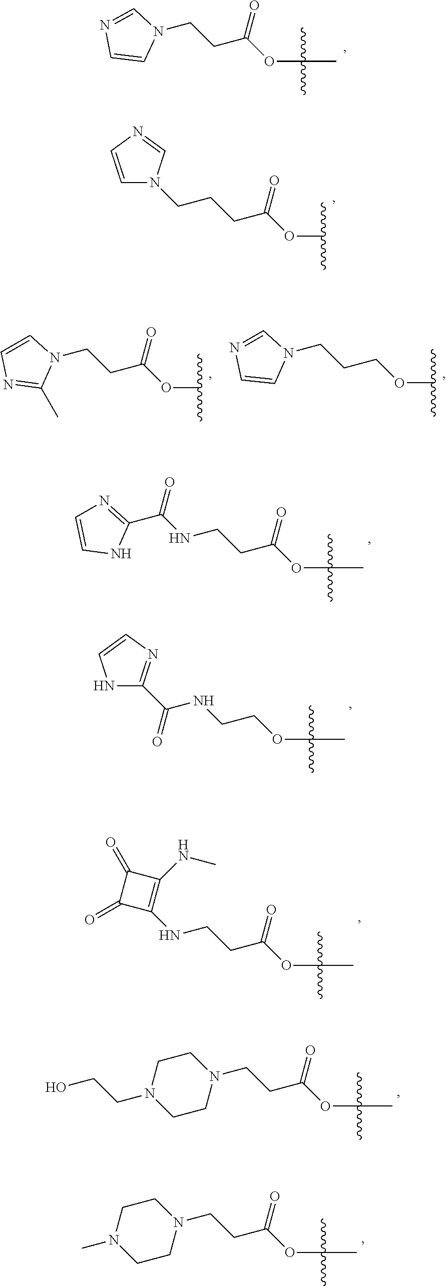

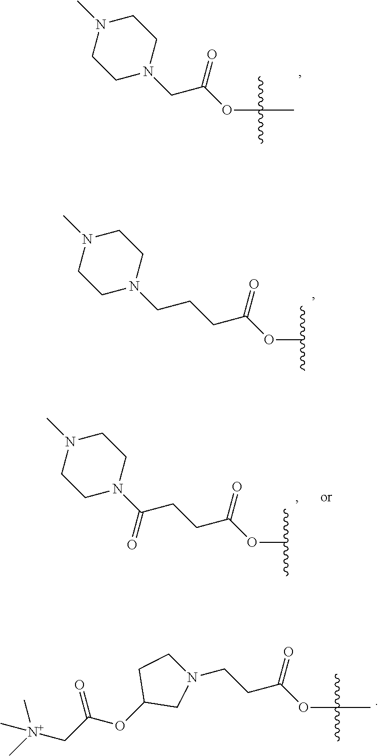 [SMILES: *OC(=O)CN1CCN(C)CC1 ; *OC(=O)CCCN1CCN(C)CC1 ; *OC(=O)CCC(=O)N1CCN(C)CC1 ; *OC(=O)CCN1CCC(OC(=O)C[N+](C)(C)C)C1]

In some embodiments, in any of the formulas described herein, X is absent, —O—, or —C(O)—.

In some embodiments, in any of the formulas described herein, Z is —O—, —C(O)O—, or —OC(O)—.

In some embodiments, in any of the formulas described herein, each of R₃₀, R₄₀, R₅₀, and R₆₀ is H or C₁-C₄ branched or unbranched alkyl.

In some embodiments, in any of the formulas described herein, each of R₃₀, R₄₀, R₅₀, and R₆₀ is H.

In any of the formulas described herein, in some embodiments, each of R₇₀ and R₈₀ is H; and R₉₀ is C₁-C₁₅ branched or unbranched alkyl, C₁-C₁₅ branched or unbranched alkenyl, cycloalkyl or substituted cycloalkyl. In some embodiments, R₉₀ is C₁-C₁₅ branched or unbranched alkyl, C₁-C₁₅ branched or unbranched alkenyl. In some embodiments, R₉₀ is C₁-C₁₅ branched or unbranched alkyl. In some embodiments, R₉₀ is C₁-C₁₂ branched or unbranched alkyl. In some embodiments, R₉₀ is C₁-C₈ branched or unbranched alkyl.

In any of the formulas described herein, in some embodiments, R₇₀ is H; and each of R₈₀ and R₉₀ is independently H, C₁-C₁₅ branched or unbranched alkyl, C₁-C₁₅ branched or unbranched alkenyl, or cycloalkyl or substituted cycloalkyl, provided that at least one of R₈₀ and R₉₀ is not H. In some embodiments, each of R₈₀ and R₉₀ is independently H, C₁-C₁₅ branched or unbranched alkyl, or C₁-C₁₅ branched or unbranched alkenyl. In some embodiments, each of R₈₀ and R₉₀ is independently H or C₁-C₁₅ branched or unbranched alkyl. In some embodiments, each of R₈₀ and R₉₀ is independently H or C₁-C₁₂ branched or unbranched alkyl. In some embodiments, each of R₈₀ and R₉₀ is independently H or C₁-C₈ branched or unbranched alkyl.

In any of the formulas described herein, in some embodiments, R₁₀₀ is H; and each of R₁₁₀ and R₁₂₀ is independently H, C₁-C₁₅ branched or unbranched alkyl, C₁-C₁₅ branched or unbranched alkenyl, or cycloalkyl or substituted cycloalkyl, provided that at least one of R₁₁₀ and R₁₂₀ is not H. In some embodiments, each of R₁₁₀ and R₁₂₀ is independently H or C₁-C₁₅ branched or unbranched alkyl, or C₁-C₁₅ branched or unbranched alkenyl. In some embodiments, each of R₁₁₀ and R₁₂₀ is independently H or C₁-C₁₅ branched or unbranched alkyl. In some embodiments, each of R₁₁₀ and R₁₂₀ is independently H or C₁-C₁₂ branched or unbranched alkyl. In some embodiments, each of R₁₁₀ and R₁₂₀ is independently H or C₁-C₈ branched or unbranched alkyl.

In any of the formulas described herein, in some embodiments, 1 is from 3 to 10, from 3 to 7, or from 4 to 7. In some embodiments, l is 4, 5, 6, 7, 8, 9 or 10. In some embodiments, l is 3, 4, 5, 6, or 7. In some embodiments, l is 4, 5, 6, or 7.

In any of the formulas described herein, in some embodiments, m is from 4 to 10, from 5 to 8, from 1 to 7, from 3 to 7, or from 1 to 5. In some embodiments, m is 4, 5, 6, 7, 8, 9 or 10. In some embodiments, m is 1, 2, 3, 4, or 5. In some embodiments, m is 3, 4, or 5. In some embodiments, m is 5, 6, 7, or 8.

In any of the formulas described herein, in some embodiments, R₇₀ is H. In some embodiments, R₁₀₀ is H.

In any of the formulas described herein, in some embodiments,

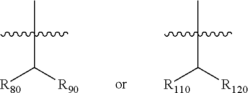

is independently selected from:

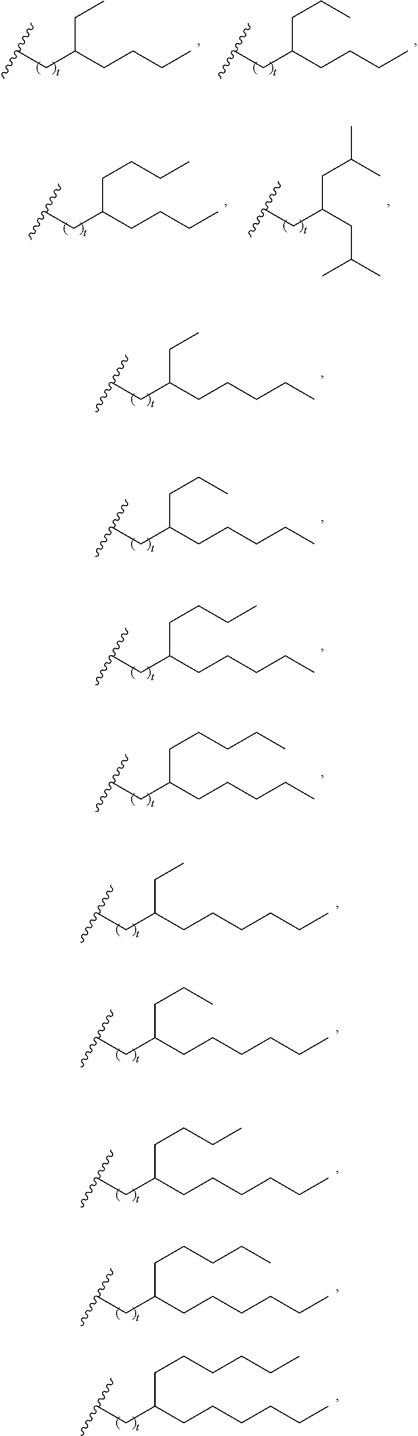

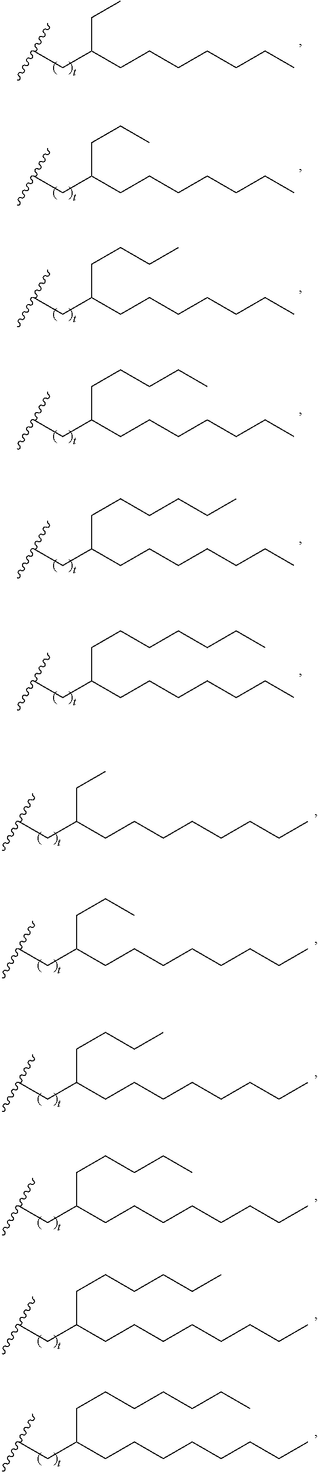

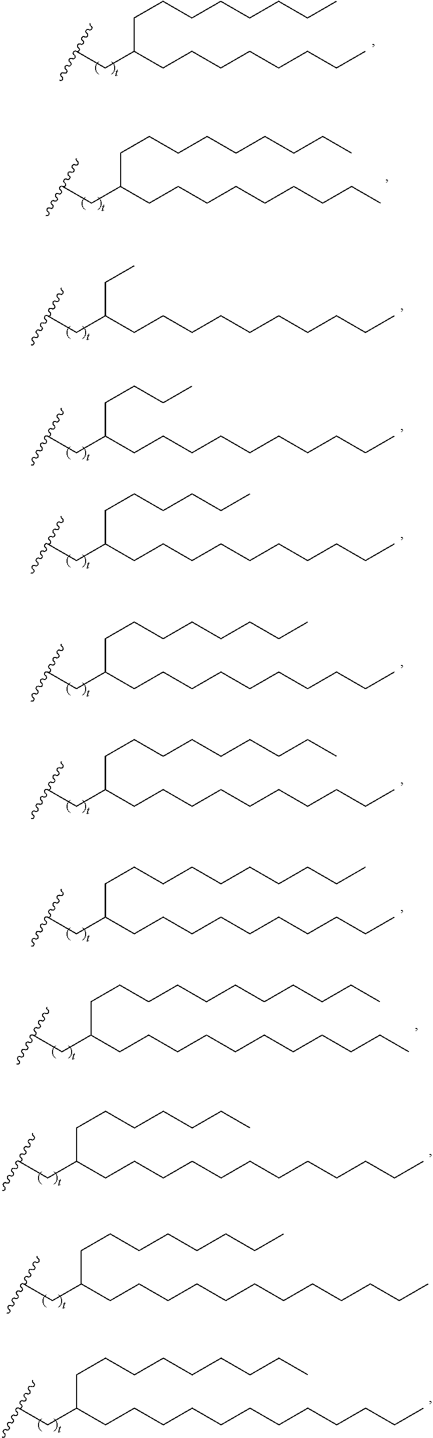

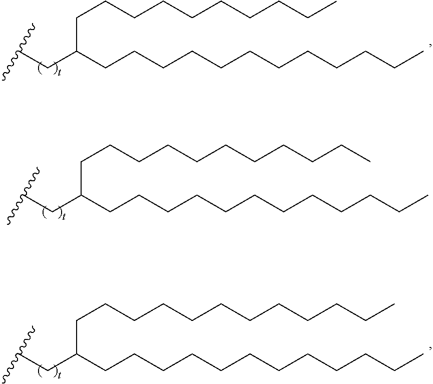

wherein t is 0, 1, 2, 3, 4, or 5.

In any of the formulas described herein, in some embodiments, M is —OC(O)—, —C(O)O—, —N(R⁷)C(O)—, —C(O)N(R⁷)—, —C(O—R₁₃)—O—, —C(O)O(CH₂)ᵣ—, —C(O)N(R⁷) (CH₂)ᵣ—, or —C(O—R₁₃)—O—(CH₂)ᵣ—, wherein each R⁷ is independently H, alkyl, alkenyl, cycloalkyl, hydroxyalkyl, or aminoalkyl; R₁₃ is branched or unbranched C₃-C₁₀ alkyl, and r is 1, 2, 3, 4, or 5. In some embodiments, M is —OC(O)— or —C(O)O—.

In any of the formulas described herein, in some embodiments, X is absent, —O—, or —C(O)—; Z is —O—, —C(O)O—, or —OC(O)—; M is —OC(O)— or —C(O)O—; Y or

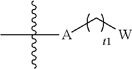

is:

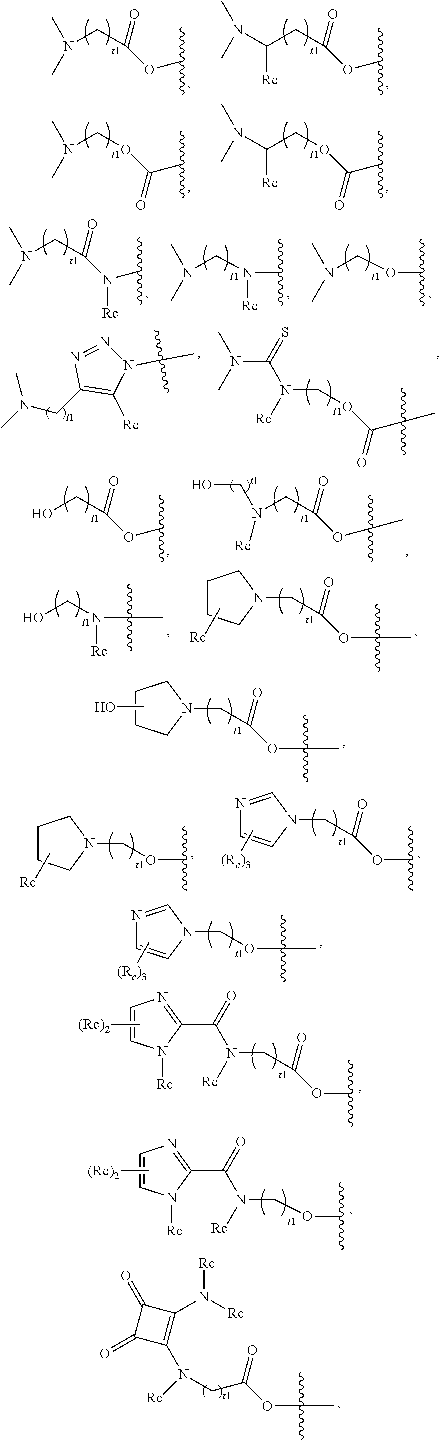

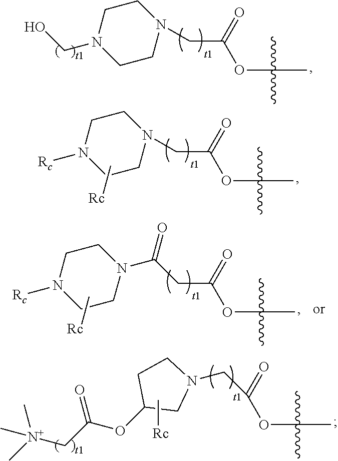

each R^{c} is independently H or C₁-C₃ alkyl; each t1 is independently 1, 2, 3, or 4; each of R₃₀, R₄₀, R₅₀, and R₆₀ is H or C₁-C₄ branched or unbranched alkyl; R₇₀ is H; and each of R₈₀ and R₉₀ is independently H or C₁-C₁₂ branched or unbranched alkyl; R₁₀₀ is H; and each of R₁₁₀ and R₁₂₀ is independently H or C₁-C₁₂ branched or unbranched alkyl, provided that at least one of R₈₀ and R₉₀ is not H, and at least one of R₁₁₀ and R₁₂₀ is not H; l is from 3 to 7; and m is from 1 to 5.

In some embodiments, disclosed herein include the lipid compounds having the formula:

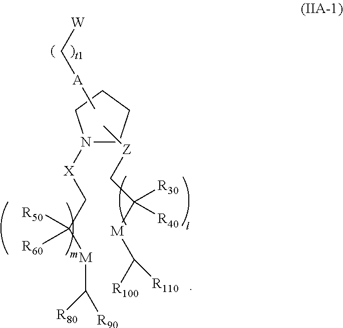

All the variables in this formula have been defined and exemplified as those described in the above embodiments.

In some embodiments, disclosed herein include the lipid compounds having the formula:

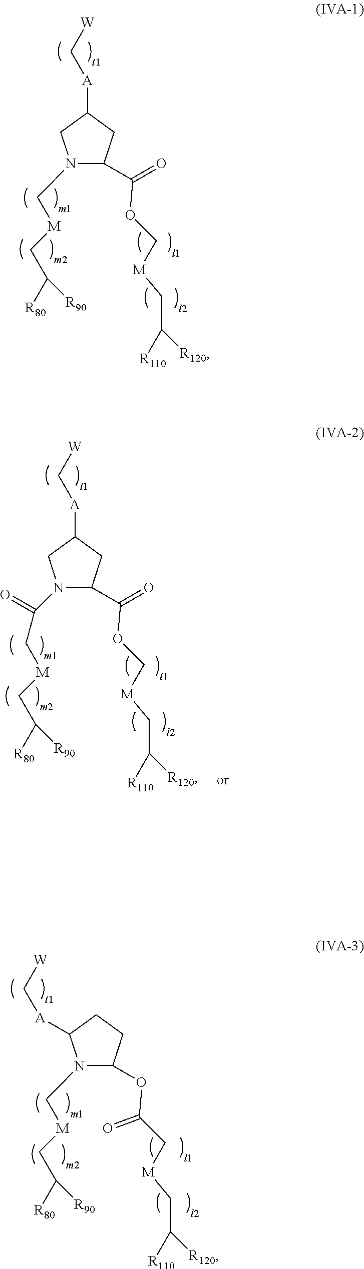

wherein:
each m1 is independently an integer from 3 to 6,
each 11 is independently an integer from 4 to 8,
m2 and 12 are each independently an integer from 0 to 3,
R₈₀ and R₉₀ are each independently unsubstituted C₅-C₈alkyl; or R₈₀ is H or unsubstituted C₁-C₄ alkyl, and R₉₀ is unsubstituted C₅-C₁₁ alkyl; and
R₁₁₀ and R₁₂₀ are each independently unsubstituted C₅-C₈alkyl; or R₁₁₀ is H or unsubstituted C₁-C₄ alkyl, and R₁₂₀ is unsubstituted C₅-C₁₁ alkyl. All the other variables in these formulas have been defined and exemplified as those described in the above embodiments.

In some embodiments, in these formulas, R₈₀ is H or unsubstituted C₁-C₂ alkyl, and R₉₀ is unsubstituted C₆-C₁₀ alkyl; and R₁₁₀ and R₁₂₀ are each independently unsubstituted C₅-C₈alkyl. In some embodiments, R₈₀, R₉₀, R₁₁₀, and R₁₂₀ are each independently unsubstituted C₅-C₈alkyl.

In some embodiments, disclosed herein include the lipid compounds having the formula:

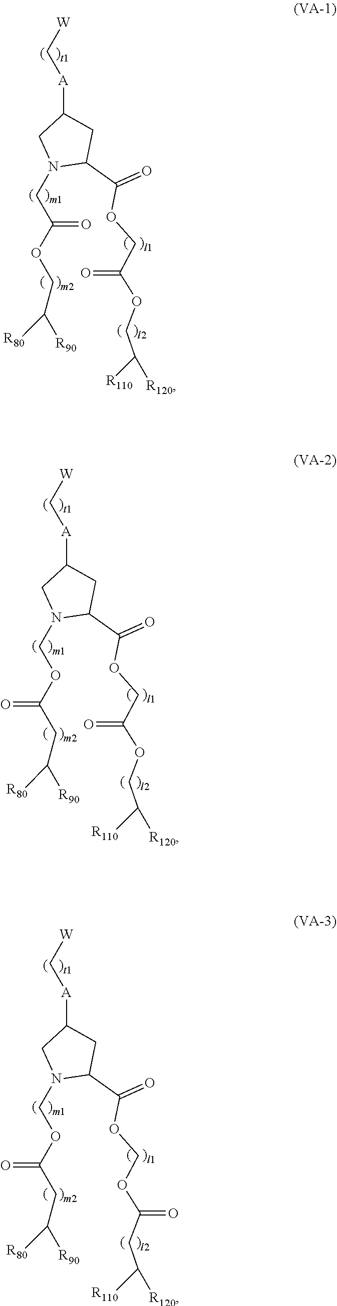

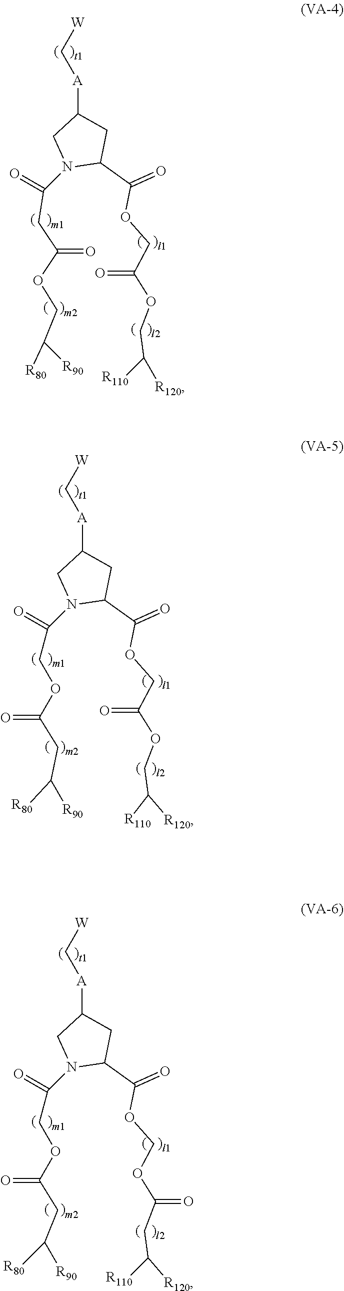

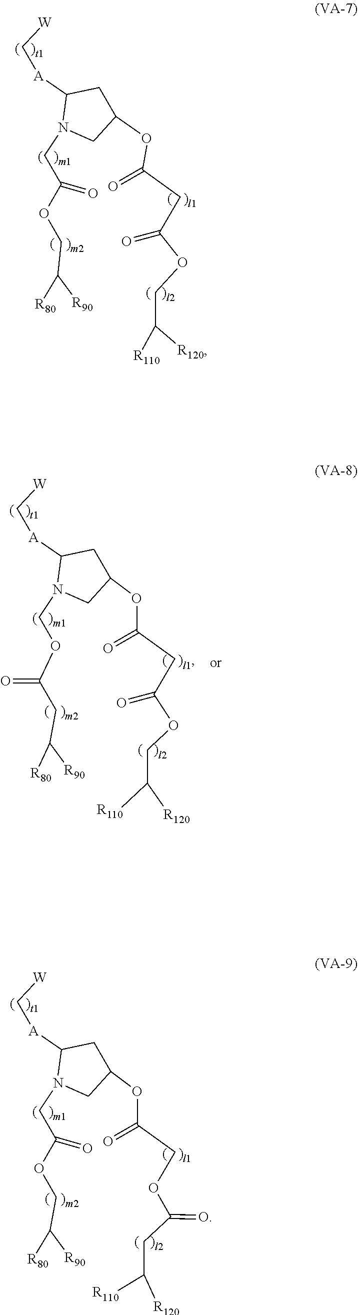

All the variables in these formulas have been defined and exemplified as those described in the above embodiments. In some embodiments, in these formulas, R₈₀ is H or unsubstituted C₁-C₂ alkyl, and R₉₀ is unsubstituted C₆-C₁₀ alkyl; and R₁₁₀ and R₁₂₀ are each independently unsubstituted C₅-C₈ alkyl. In some embodiments, R₈₀, R₉₀, R₁₁₀, and R₁₂₀ are each independently unsubstituted C₅-C₈ alkyl. In some embodiments,

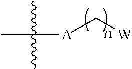

### is: OH,

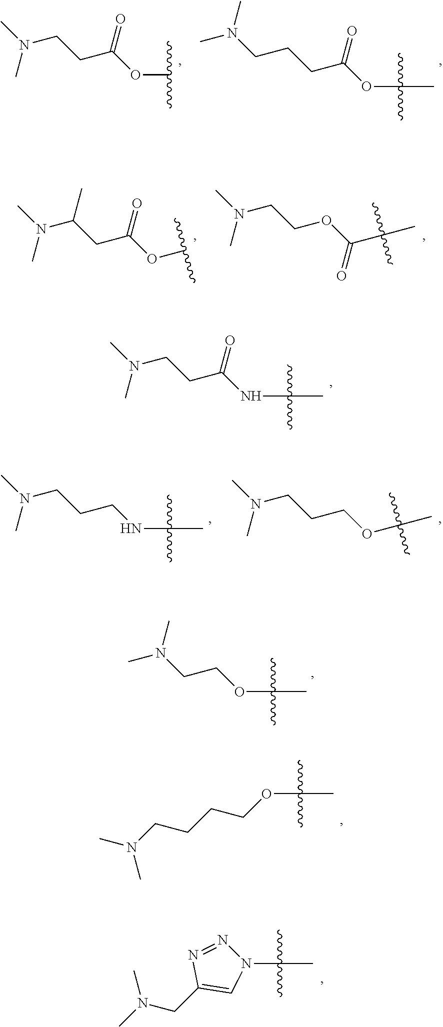

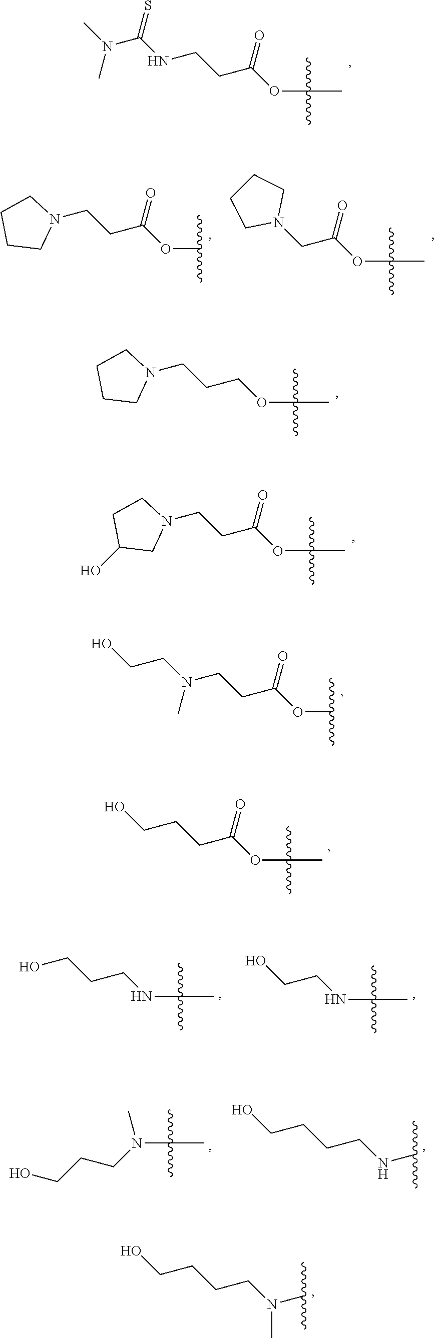

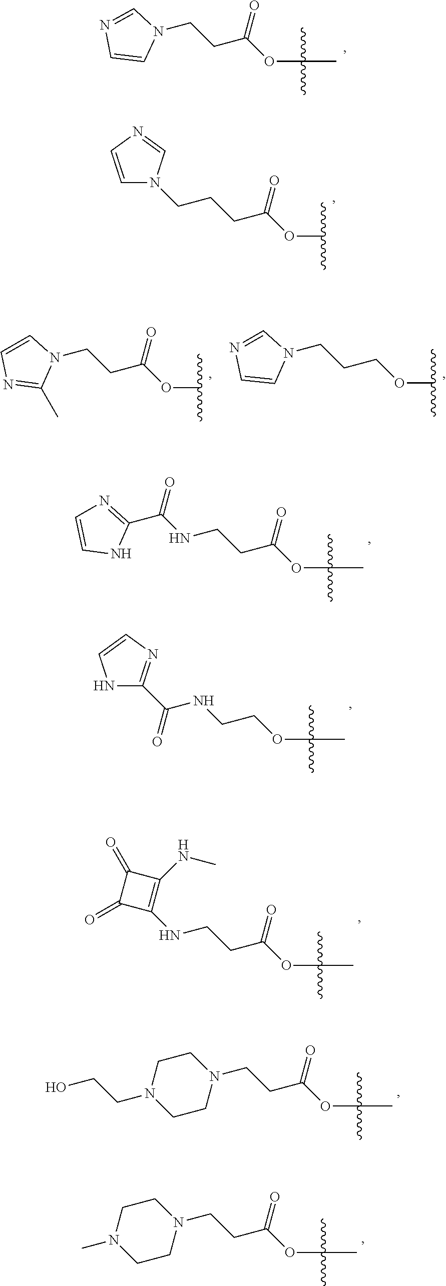

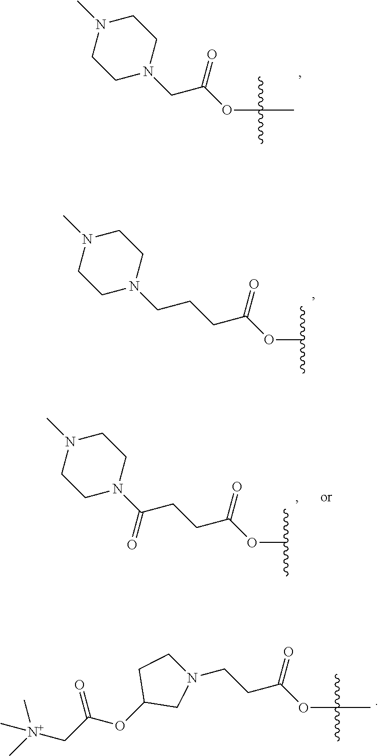 [SMILES: *OC(=O)CN1CCN(C)CC1 ; *OC(=O)CCCN1CCN(C)CC1 ; *OC(=O)CCC(=O)N1CCN(C)CC1 ; *OC(=O)CCN1CCC(OC(=O)C[N+](C)(C)C)C1]

In some embodiments, disclosed herein include the lipid compounds having the formula:

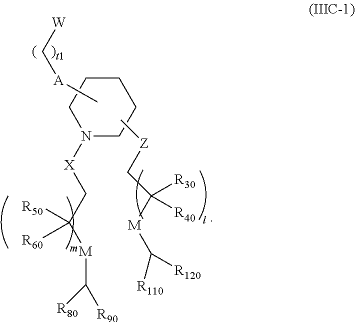

All the variables in this formula have been defined and exemplified as those described in the above embodiments.

In some embodiments, disclosed herein include the lipid compounds having the formula:

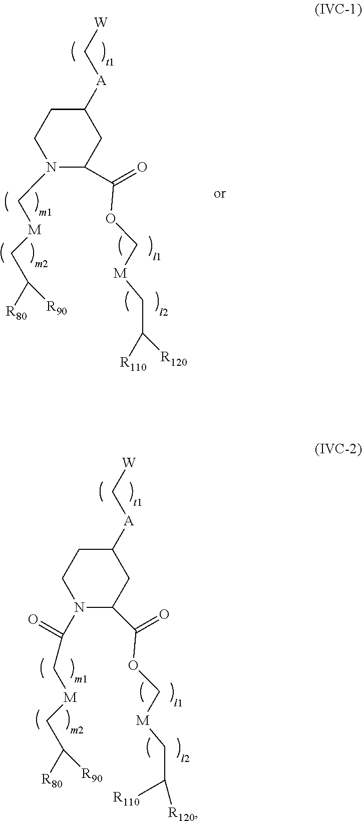

wherein:
each m1 is independently an integer from 3 to 6,
each 11 is independently an integer from 4 to 8,
m2 and 12 are each independently an integer from 0 to 3,
R₈₀ and R₉₀ are each independently unsubstituted C₅-C₈ alkyl; or R₈₀ is H or unsubstituted C₁-C₄ alkyl, and R₉₀ is unsubstituted C₅-C₁₁ alkyl; and
R₁₁₀ and R₁₂₀ are each independently unsubstituted C₅-C₈ alkyl; or R₁₁₀ is H or unsubstituted C₁-C₄ alkyl, and R₁₂₀ is unsubstituted C₅-C₁₁ alkyl. All the other variables in these formulas have been defined and exemplified as those described in the above embodiments. In some embodiments, in these formulas, R₈₀ is H or unsubstituted C₁-C₂ alkyl, and R₉₀ is unsubstituted C₆-C₁₀ alkyl; and R₁₁₀ and R₁₂₀ are each independently unsubstituted C₅-C₈alkyl. In some embodiments, R₈₀, R₉₀, R₁₁₀, and R₁₂₀ are each independently unsubstituted C₅-C₈alkyl.

In some embodiments, disclosed herein include the lipid compounds having the formula:

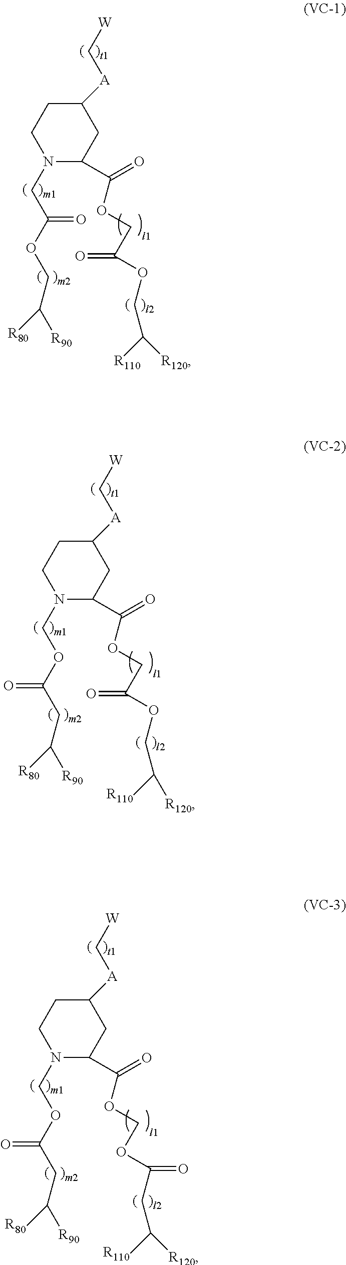

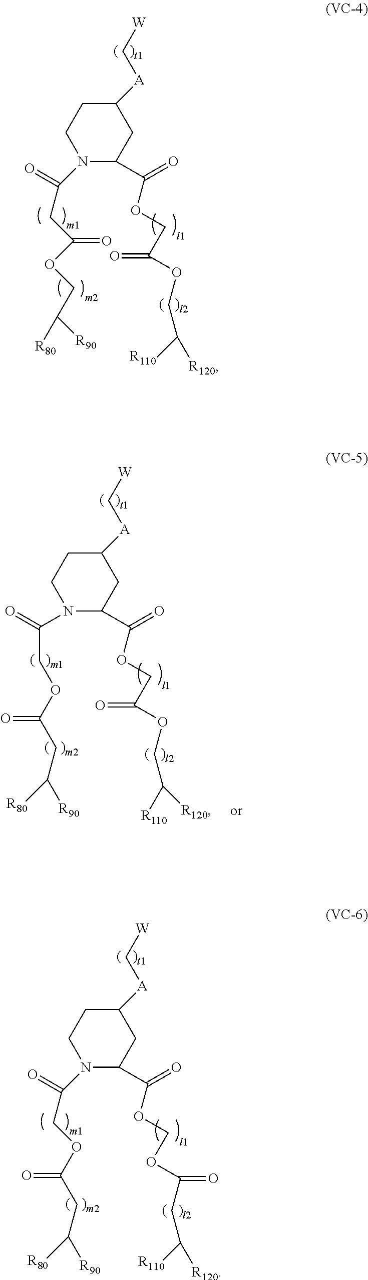

All the variables in these formulas have been defined and exemplified as those described in the above embodiments. In some embodiments, in these formulas, R₈₀ is H or unsubstituted C₁-C₂ alkyl, and R₉₀ is unsubstituted C₆-C₁₀ alkyl; and R₁₁₀ and R₁₂₀ are each independently unsubstituted C₅-C₈ alkyl. In some embodiments, R₈₀, R₉₀, R₁₁₀, and R₁₂₀ are each independently unsubstituted C₅-C₈ alkyl. In some embodiments,

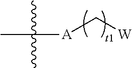

### is: OH,

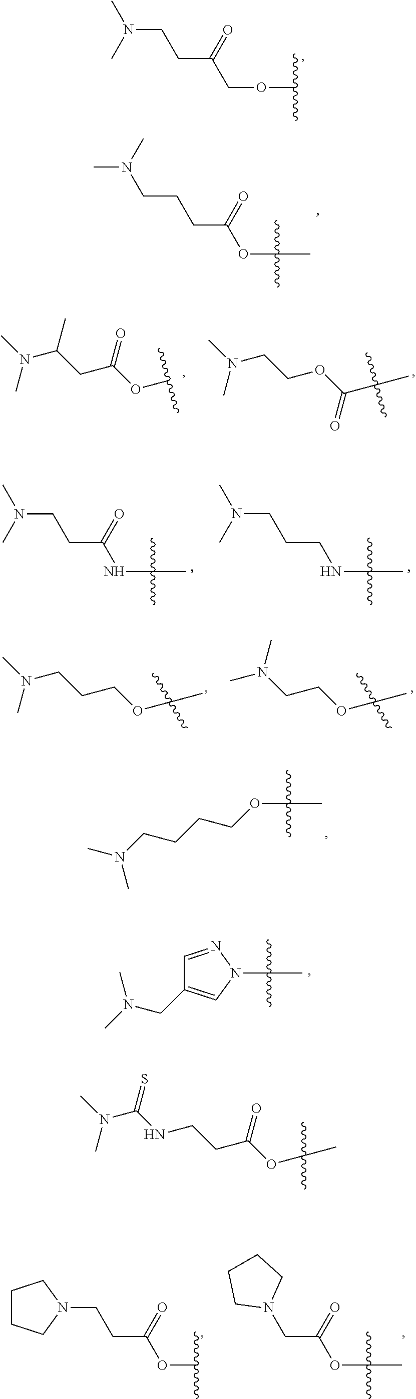

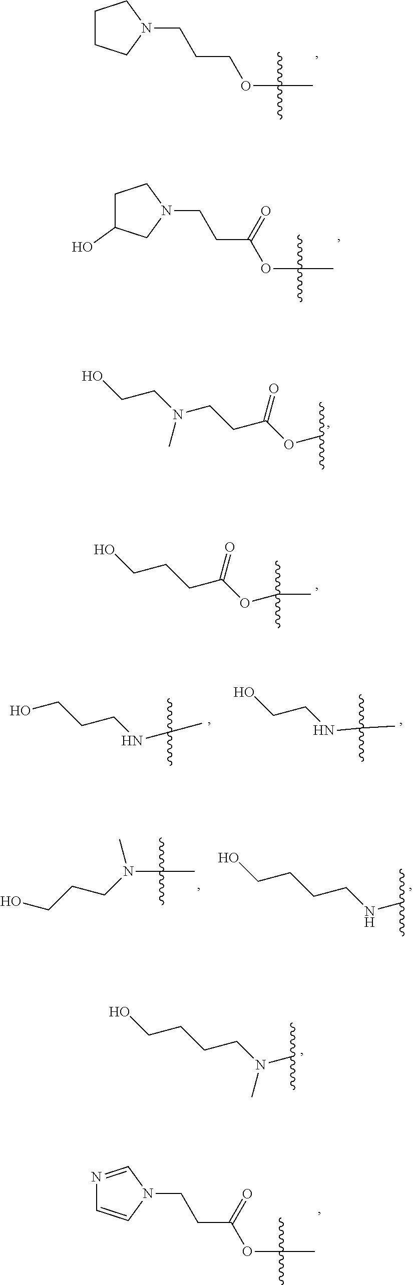

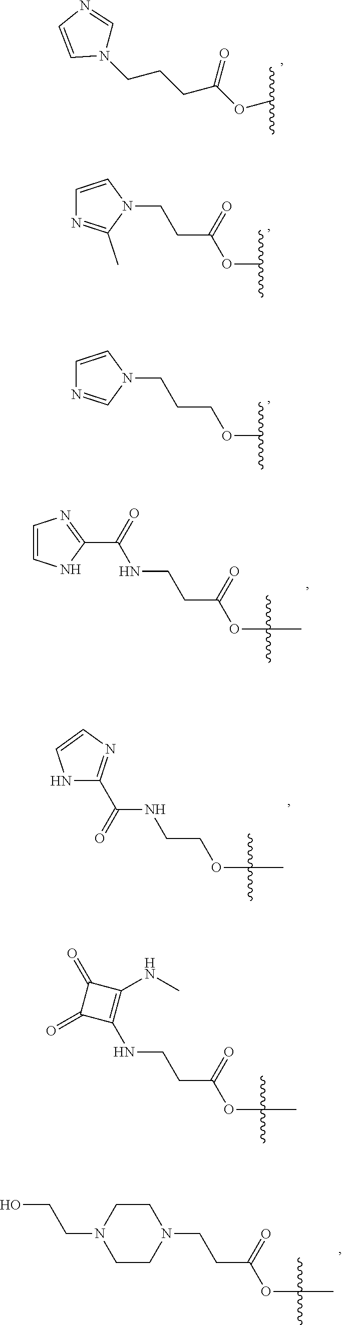

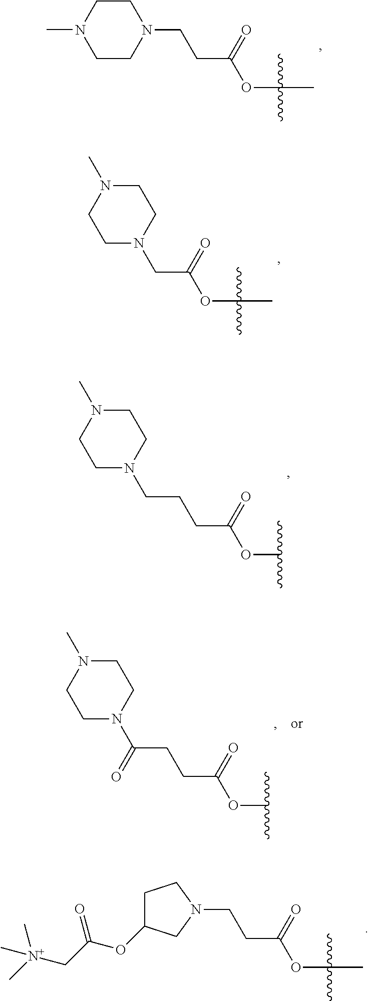 [SMILES: *OC(=O)CCN1CCN(C)CC1 ; *OC(=O)CN1CCN(C)CC1 ; *OC(=O)CCCN1CCN(C)CC1 ; *OC(=O)CCC(=O)N1CCN(C)CC1 ; *OC(=O)CCN1CCC(OC(=O)C[N+](C)(C)C)C1]

In some embodiments, the disclosure relates to ionizable lipids of Formula (IIA):

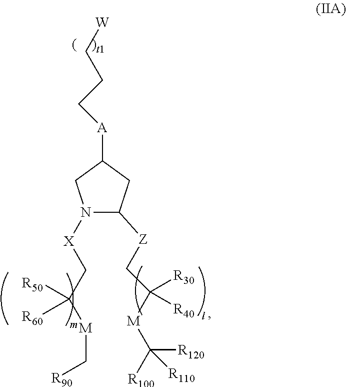

pharmaceutically acceptable salts thereof, and stereoisomers of any of the foregoing, wherein:
A is absent, —O—, —N(R⁷)—, —O-alkylene-, -alkylene-O—, —OC(O)—, —C(O)O—, —NHC(O)—, —C(O)N(R⁷)—, —N(R⁷)C(O)N(R⁷)—, —S—, —S—S—;
X is absent, —O—, —CO—, —N(R⁷)—, —O-alkylene-, -alkylene-O—, —OC(O)—, —C(O)O—, —NHC(O)—, —C(O)N(R⁷)—, or —S—;
Z is absent, —O—, —N(R⁷)—, —O-alkylene-, -alkylene-O—, —OC(O)—, —C(O)O—, —NHC(O)—, —C(O)NH—, or —S—;
each R⁷ is independently H, C₁-C₃ branched or unbranched alkyl, C₂-C₃ branched or unbranched alkenyl, cycloalkyl, hydroxyalkyl, or aminoalkyl;
each M is independently a biodegradable moiety;
each of R₃₀, R₄₀, R₅₀, R₆₀, R₁₀₀, R₁₁₀, and R₁₂₀ is independently H, C₁-C₁₆ branched or unbranched alkyl, or C₁-C₁₆ branched or unbranched alkenyl, optionally interrupted with heteroatom or substituted with OH, SH, or halogen;
R₉₀ is C₁-C₁₅ branched or unbranched alkyl, C₁-C₁₅ branched or unbranched alkenyl, or cycloalkyl or substituted cycloalkyl;
t is 0, 1, 2, or 3;
l is an integer from 1 to 10;
m is an integer from 1 to 10; and
W is hydroxyl, hydroxyalkyl, or one of the following moieties:

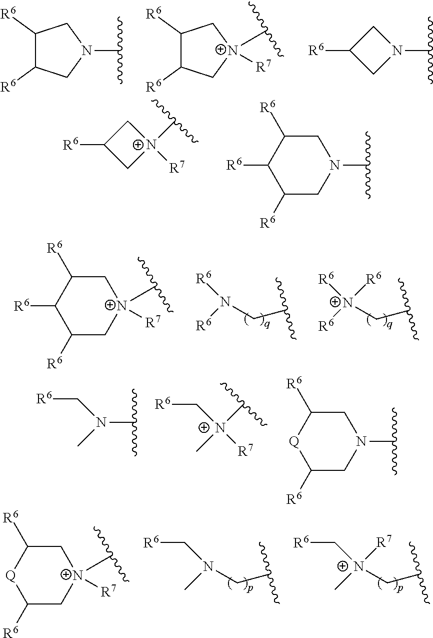

wherein
   Q is —O— or —N(R⁷)
   R⁶ is independently H, alkyl, hydroxyalkyl, amino, aminoalkyl, thiol, or thioalkyl;
   each R⁸ is independently H, alkyl, hydroxyalkyl, amino, aminoalkyl, thiol, or thiolalkyl, or two R⁸ may form a ring;
   q is 0, 1, 2, 3, 4, or 5; and
   p is 0, 1, 2, 3, 4, or 5.

In some embodiments, the disclosure to ionizable lipids of Formula (IIIA):

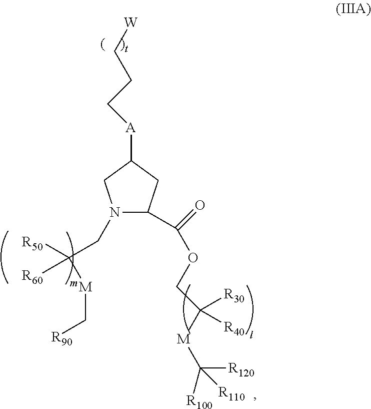

pharmaceutically acceptable salts thereof, and stereoisomers of any of the foregoing, wherein the definitions of the variables in (IIIA) are the same as those in (IIA).

In some embodiments, the disclosure relates to ionizable lipids of Formula (IIB):

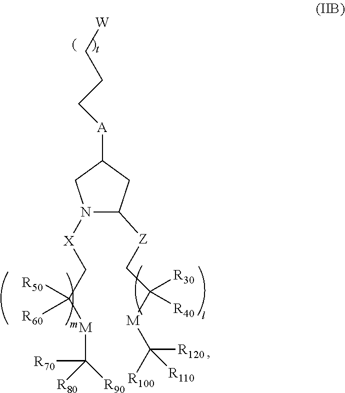

pharmaceutically acceptable salts thereof, and stereoisomers of any of the foregoing, wherein:
A is absent, —O—, —N(R⁷)—, —O-alkylene-, -alkylene-O—, —OC(O)—, —C(O)O—, —NHC(O)—, —C(O)N(R⁷)—, —N(R⁷)C(O)N(R⁷)—, —S—, —S—S—;
X is absent, —O—, —CO—, —N(R⁷)—, —O-alkylene-, -alkylene-O—, —OC(O)—, —C(O)O—, —NHC(O)—, —C(O)N(R⁷)—, or —S—;
Z is absent, —O—, —N(R⁷)—, —O-alkylene-, -alkylene-O—, —OC(O)—, —C(O)O—, —NHC(O)—, —C(O)NH—, or —S—;
each R⁷ is independently H, C₁-C₃ branched or unbranched alkyl, C₂-C₃ branched or unbranched alkenyl, cycloalkyl, hydroxyalkyl, or aminoalkyl;
each M is independently a biodegradable moiety;
each of R₃₀, R₄₀, R₅₀, R₆₀, R₇₀, R₈₀, R₉₀, R₁₀₀, R₁₁₀, and R₁₂₀ is independently H, C₁-C₁₆ branched or unbranched alkyl, or C₁-C₁₆ branched or unbranched alkenyl, optionally interrupted with heteroatom or substituted with OH, SH, or halogen;
t is 0, 1, 2, or 3;
l is an integer from 1 to 10;
m is an integer from 1 to 10; and
W is hydroxyl, hydroxyalkyl, or one of the following moieties:

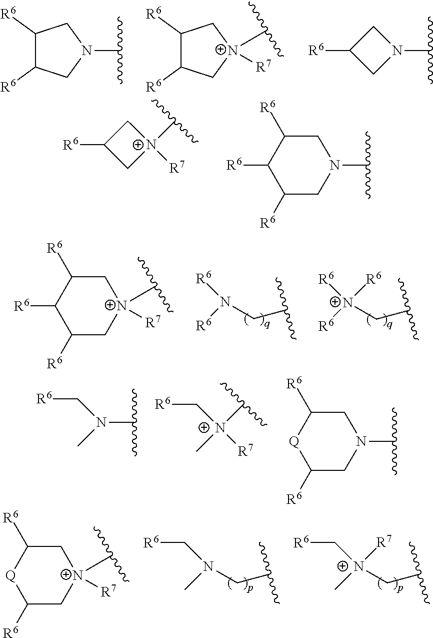

wherein
   Q is —O— or —N(R⁷);
   R⁶ is independently H, alkyl, hydroxyalkyl, amino, aminoalkyl, thiol, or thiolalkyl;
   each R⁸ is independently H, alkyl, hydroxyalkyl, amino, aminoalkyl, thiol, or thiolalkyl, or two R⁸ may form a ring;
   q is 0, 1, 2, 3, 4, or 5; and
   p is 0, 1, 2, 3, 4, or 5.

In some embodiments, the disclosure relates to ionizable lipids of Formula (IIIB):

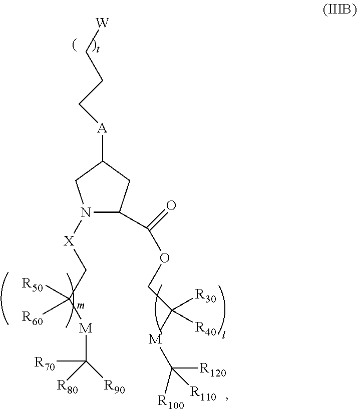

pharmaceutically acceptable salts thereof, and stereoisomers of any of the foregoing, wherein the definitions of the variables in (IIIB) are the same as those in (IIB).

In some embodiments, the disclosure relates to ionizable lipids of Formula (IIC):

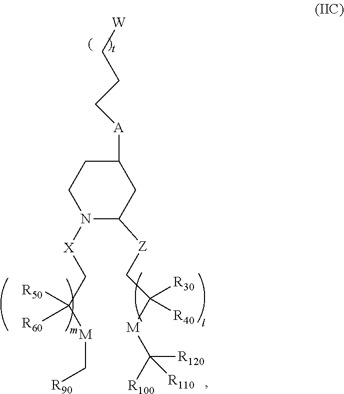

pharmaceutically acceptable salts thereof, and stereoisomers of any of the foregoing, wherein:
A is absent, —O—, —N(R⁷)—, —O-alkylene-, -alkylene-O—, —OC(O)—, —C(O)O—, —N(R⁷)C(O)—, —C(O)N(R′)—, N(R⁷)C(O)N(R⁷)—, —S—, —S—S—;
each of R₃₀, R₄₀, R₅₀, R₆₀, R₁₀₀, R₁₁₀ and R₁₂₀ is independently H, C₁-C₁₆ branched or unbranched alkyl or C₁-C₁₆ branched or unbranched alkenyl, optionally interrupted with heteroatom or substituted with OH, SH, or halogen;
R₉₀ is C₁-C₁₅ branched or unbranched alkyl, C₁-C₁₅ branched or unbranched alkenyl, cycloalkyl or substituted cycloalkyl;
each R⁷ is independently H, C₁-C₃ branched or unbranched alkyl, C₂-C₃ branched or unbranched alkenyl, cycloalkyl, hydroxyalkyl, or aminoalkyl;
each M is independently a biodegradable moiety;
t is 0, 1, 2, or 3;
l is an integer from 1 to 10;
m is an integer from 1 to 10; and
W is hydroxyl, hydroxyalkyl, or one of the following moieties:

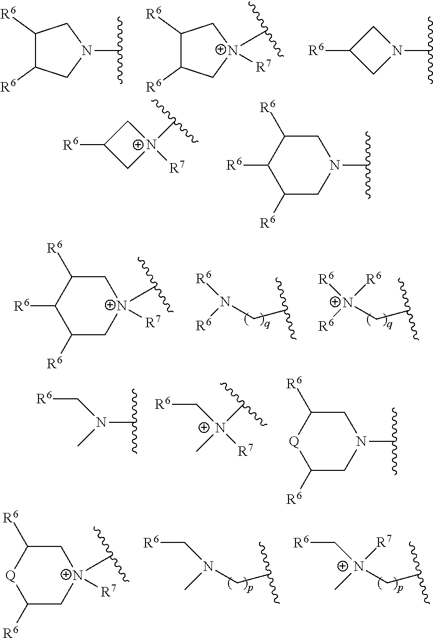

wherein
   Q is —O— or —N(R⁷)—;
   R⁶ is independently H, alkyl, hydroxyalkyl, amino, aminoalkyl, thiol, or thiolalkyl;
   each R⁸ is independently H, alkyl, hydroxyalkyl, amino, aminoalkyl, thiol, or thiolalkyl, or two R⁸ may form a ring;
   q is 0, 1, 2, 3, 4, or 5; and
   p is 0, 1, 2, 3, 4 or 5.

In some embodiments, the disclosure relates to ionizable lipids of Formula (IIIC):

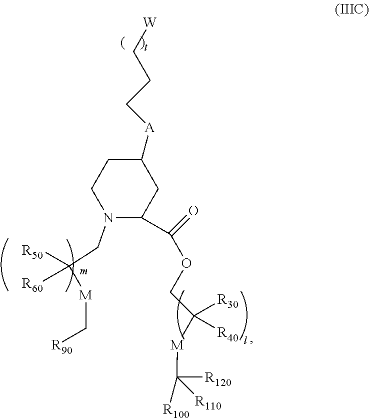

pharmaceutically acceptable salts thereof, and stereoisomers of any of the foregoing, wherein the definitions of the variables in (IIIA) are the same as those in (IIC).

In some embodiments, the disclosure relates to ionizable lipids of Formula (IIID):

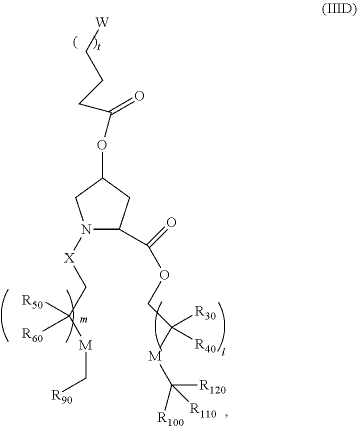

pharmaceutically acceptable salts thereof, and stereoisomers of any of the foregoing, wherein the definitions of the variables in (IID) are the same as those defined above.

In some embodiments, the disclosure relates to ionizable lipids of Formula (IIIE):

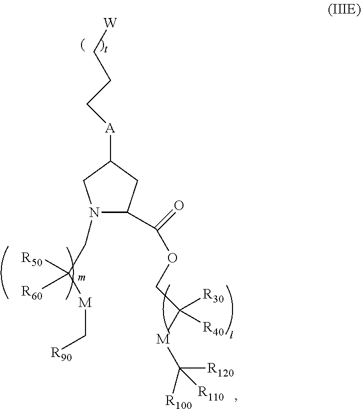

pharmaceutically acceptable salts thereof, and stereoisomers of any of the foregoing, wherein the definitions of the variables in (IID) are the same as those defined above.

Embodiments regarding various variables in the formulas (IIA), (IIB), (IIC), (IIIA), (IIIB), (IIIC), (IIID), or (IIIE) are further discussed below.

In some embodiments, X is absent, —O—, or —C(O)—. In one embodiment, X is absent. In one embodiment, X is —O—. In one embodiment, X is —C(O)—.

In some embodiments, Z is —O—, —C(O)O—, or —OC(O)—. In one embodiment, Z is —O—. In one embodiment, Z is —C(O)O— or —OC(O)—.

In some embodiments, each of R₃₀, R₄₀, R₅₀, and R₆₀ is H or C₁-C₄ branched or unbranched alkyl.

In some embodiments, each of R₃₀, R₄₀, R₅₀, and R₆₀ is H.

In some embodiments, each of R₇₀ and R₈₀ is H; and R₉₀ is C₁-C₁₅ branched or unbranched alkyl, C₁-C₁₅ branched or unbranched alkenyl. In some embodiments, R₉₀ is C₁-C₁₅ branched or unbranched alkyl. In some embodiments R₉₀ is C₁-C₁₂ branched or unbranched alkyl.

In some embodiments, R₇₀ is H; and each of R₈₀ and R₉₀ is independently C₁-C₁₅ branched or unbranched alkyl, C₁-C₁₅ branched or unbranched alkenyl. In some embodiments, each of R₈₀ and R₉₀ is independently C₁-C₁₅ branched or unbranched alkyl. In some embodiments, each of R₈₀ and R₉₀ is independently C₁-C₁₂ branched or unbranched alkyl. In some embodiments, each of R₈₀ and R₉₀ is independently C₁-C₈ branched or unbranched alkyl.

In some embodiments, R₁₀₀ is H; and each of R₁₁₀ and R₁₂₀ is independently C₁-C₁₅ branched or unbranched alkyl, C₁-C₁₅ branched or unbranched alkenyl. In some embodiments, each of R₁₁₀ and R₁₂₀ is independently C₁-C₁₅ branched or unbranched alkyl. In some embodiments, each of R₁₁₀ and R₁₂₀ is independently C₁-C₁₂ branched or unbranched alkyl. In some embodiments, each of R₁₁₀ and R₁₂₀ is independently C₁-C₈ branched or unbranched alkyl.

In some embodiments, l is from 3 to 10, from 3 to 7, or from 4 to 7. In some embodiments, l is 4, 5, 6, 7, 8, 9 or 10. In some embodiments, l is 4, 5, 6, or 7.

In some embodiments, m is from 4 to 10, from 5 to 8, from 1 to 7, from 3 to 7, or from 1 to 5. In some embodiments, m is 4, 5, 6, 7, 8, 9 or 10. In some embodiments, m is 3, 4, or 5. In some embodiments, m is 5, 6, 7, or 8.

In some embodiments, M is —OC(O)—, —C(O)O—, —N(R⁷)C(O)—, —C(O)N(R⁷)—, —C(O—R₁₃)—O—, —C(O)O(CH₂)ᵣ—, —C(O)N(R⁷) (CH₂)ᵣ—, or —C(O—R₁₃)—O—(CH₂)ᵣ—, wherein each R⁷ is independently H, alkyl, alkenyl, cycloalkyl, hydroxyalkyl, or aminoalkyl; R₁₃ is branched or unbranched C₃-C₁₀ alkyl, and r is 1, 2, 3, 4, or 5. In some embodiments, M is —OC(O)— or —C(O)O—.

In some embodiments, the pKa of the protonated form of the ionizable lipid compound described herein is about 4.5 to about 8.0, for example, about 4.6 to about 7.8, about 4.6 to about 7.3, about 4.6 to about 6.8, about 4.6 to about 6.2, about 4.6 to about 6.0, about 4.6 to about 5.9, about 4.6 to about 5.8, about 4.6 to about 5.6, about 4.6 to 5.5, about 5.7 to about 6.5, about 5.7 to about 6.4, or from about 5.8 to about 6.2. In some embodiments, the pKa of the protonated form of the ionizable lipid compound is about 4.6 to about 7.8. In some embodiments, the pKa of the protonated form of the ionizable lipid compound is about 4.6 to about 5.6. In some embodiments, the pKa of the protonated form of the ionizable lipid compound is about 5.5 to about 6.0. In some embodiments, the pKa of the protonated form of the ionizable lipid compound is about 6.1 to about 6.3. In some embodiments, the pKa of the protonated form of the ionizable lipid compound is about 4.7 to about 5.1.

Non-limiting examples of ionizable lipid compounds disclosed here are set forth in Table 1 below.

TABLE 1
Exemplary ionizable lipid compounds.

| Com- | | |
|---|---|---|
| pound | Structure | IUPAC name |
| 2310 | 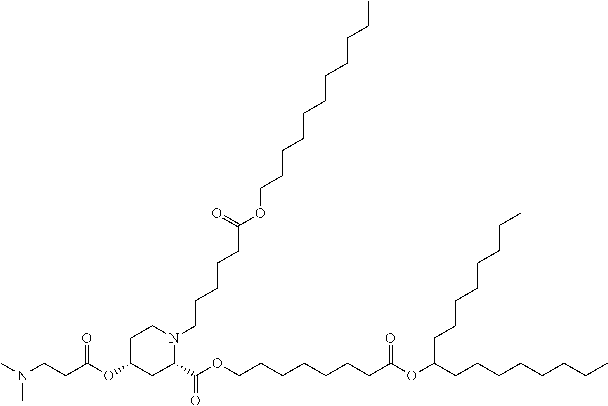 [SMILES: CCCCCCCCCCCOC(=O)CCCCCN1CC[C@@H](OC(=O)CCN(C)C)C[C@H]1C(=O)OCCCCCCCC(=O)OC(CCCCCCCC)CCCCCCCC] | heptadecan- 9-yl 8-[(2S,4R)- 4-{[3- (dimethyl- amino) propanoyl] oxy}-1-[6- oxo-6- (undecyloxy) hexyl] piperidine-2- carbonyloxy] octanoate |
| 2309 | 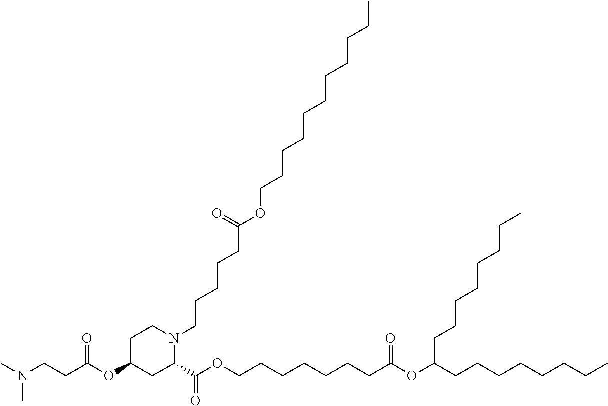 [SMILES: CCCCCCCCCCCOC(=O)CCCCCN1CC[C@H](OC(=O)CCN(C)C)C[C@H]1C(=O)OCCCCCCCC(=O)OC(CCCCCCCC)CCCCCCCC] | heptadecan- 9-yl 8-[(2S,4S)- 4-{[3- (dimethyl- amino) propanoyl] oxy}-1-[6- oxo-6- (undecyloxy) hexyl] piperidine-2- carbonyloxy] octanoate |
| 2308 | 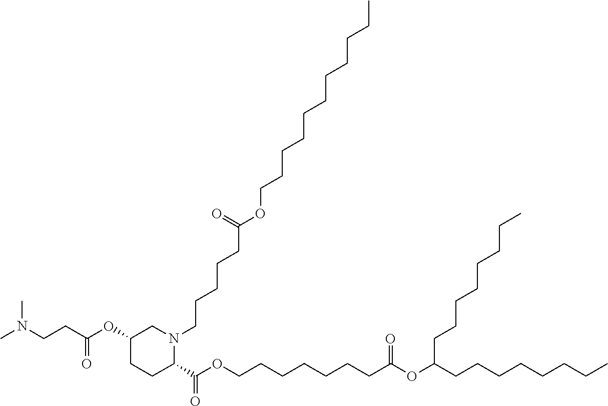 [SMILES: CCCCCCCCCCCOC(=O)CCCCCN1C[C@@H](OC(=O)CCN(C)C)CC[C@H]1C(=O)OCCCCCCCC(=O)OC(CCCCCCCC)CCCCCCCC] | heptadecan- 9-yl 8-[(2S,5S)- 5-{[3- (dimethyl- amino) propanoyl] oxy}-1-[6- oxo-6- (undecyloxy) hexyl] piperidine-2- carbonyloxy] octanoate |
| 2307 | 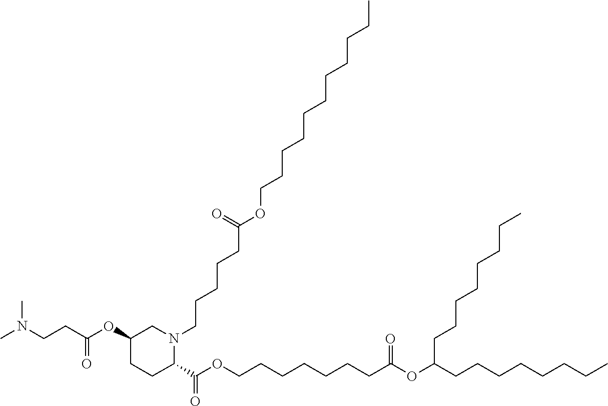 [SMILES: CCCCCCCCCCCOC(=O)CCCCCN1C[C@H](OC(=O)CCN(C)C)CC[C@H]1C(=O)OCCCCCCCC(=O)OC(CCCCCCCC)CCCCCCCC] | heptadecan- 9-yl 8-[(2S,5R)- 5-{[3- (dimethyl- amino) propanoyl] oxy}-1-[6- oxo-6- (undecyloxy) hexyl] piperidine-2- carbonyloxy] octanoate |
| 2306 | 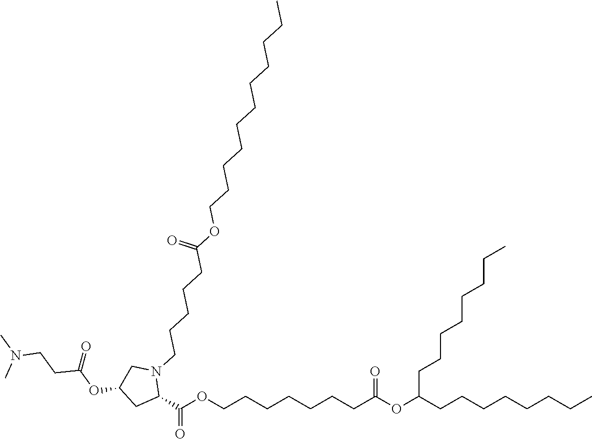 [SMILES: CCCCCCCCCCCOC(=O)CCCCCN1C[C@@H](OC(=O)CCN(C)C)C[C@H]1C(=O)OCCCCCCCC(=O)OC(CCCCCCCC)CCCCCCCC] | heptadecan- 9-yl 8-[(2S,4S)- 4-{[3- (dimethyl- amino) propanoyl] oxy}-1-[6- oxo-6- (undecyloxy) hexyl] pyrrolidine-2- carbonyloxy] octanoate |
| 2305 | 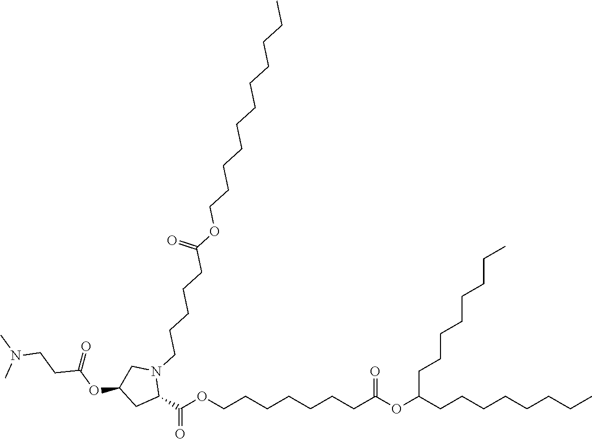 [SMILES: CCCCCCCCCCCOC(=O)CCCCCN1C[C@H](OC(=O)CCN(C)C)C[C@H]1C(=O)OCCCCCCCC(=O)OC(CCCCCCCC)CCCCCCCC] | heptadecan- 9-yl 8-[(2S,4R)- 4-{[3- (dimethyl- amino) propanoyl] oxy}-1-[6- oxo-6- (undecyloxy) hexyl] pyrrolidine-2- carbonyloxy] octanoate |
| 2304 | 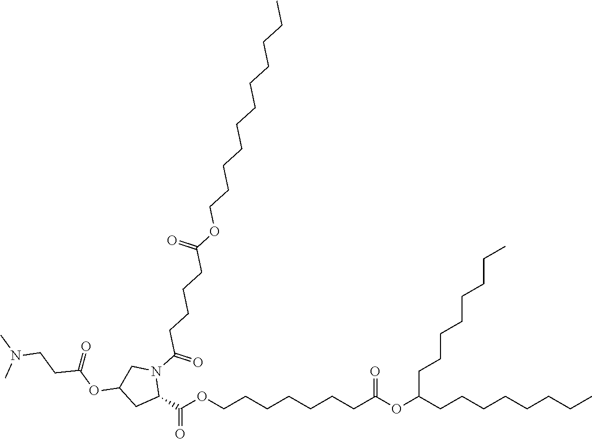 [SMILES: CCCCCCCCCCCOC(=O)CCCCC(=O)N1CC(OC(=O)CCN(C)C)C[C@H]1C(=O)OCCCCCCCC(=O)OC(CCCCCCCC)CCCCCCCC] | heptadecan- 9-yl 8-[(2S)-4-{[3- (dimethyl- amino) propanoyl] oxy}-1-[6- oxo-6- (undecyloxy) hexanoyl] pyrrolidine-2- carbonyloxy] octanoate |
| 2298 | 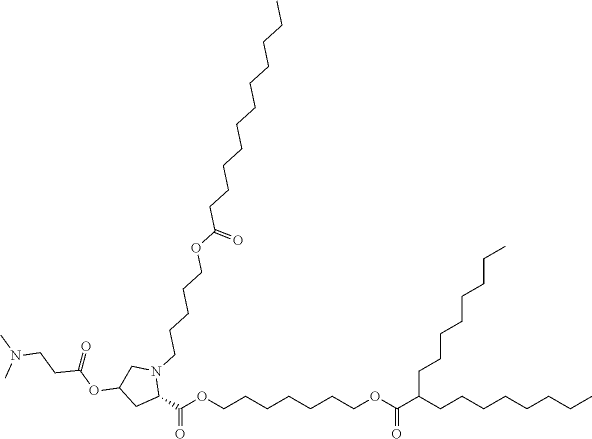 [SMILES: CCCCCCCCCCCC(=O)OCCCCCN1CC(OC(=O)CCN(C)C)C[C@H]1C(=O)OCCCCCCCOC(=O)C(CCCCCCCC)CCCCCCCC] | 5-[(2S)-4-{[3- (dimethyl- amino) propanoyl] oxy}-2- [({7-[(2- octyldecanoyl) oxy]heptyl} oxy)carbonyl] pyrrolidin-1- yl]pentyl dodecanoate |
| 2297 | 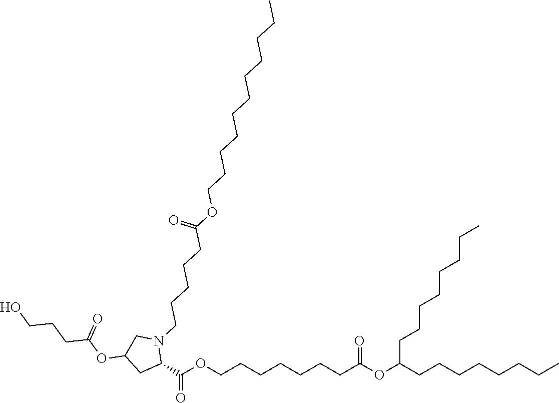 [SMILES: CCCCCCCCCCCOC(=O)CCCCCN1CC(OC(=O)CCCO)C[C@H]1C(=O)OCCCCCCCC(=O)OC(CCCCCCCC)CCCCCCCC] | heptadecan- 9-yl 8-[(2S)-4-[(4- hydroxy- butanoyl)oxy]- 1-[6-oxo-6- (undecyloxy) hexyl] pyrrolidine-2- carbonyloxy] octanoate |
| 2296 | 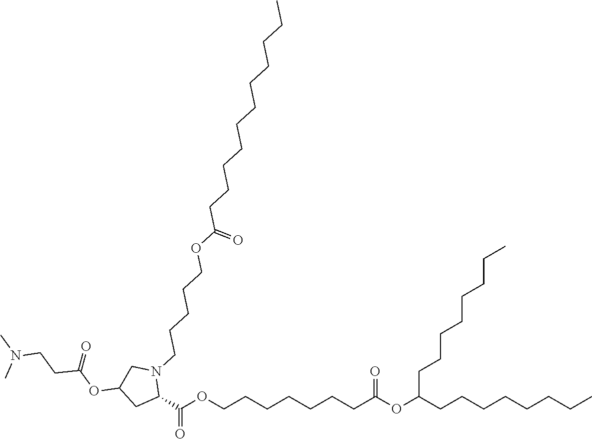 [SMILES: CCCCCCCCCCCC(=O)OCCCCCN1CC(OC(=O)CCN(C)C)C[C@H]1C(=O)OCCCCCCCC(=O)OC(CCCCCCCC)CCCCCCCC] | 5-[(2S)-4-{[3- (dimethyl- amino) propanoyl] oxy}-2-({[8- (heptadecan- 9-yloxy)-8- oxooctyl]oxy} carbonyl) pyrrolidin-1- yl]pentyl dodecanoate |
| 2295 | 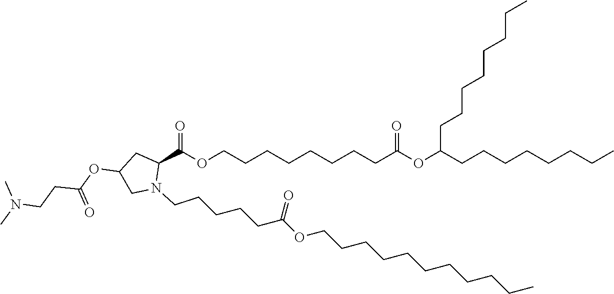 [SMILES: CCCCCCCCCCCOC(=O)CCCCCN1CC(OC(=O)CCN(C)C)C[C@H]1C(=O)OCCCCCCCCC(=O)OC(CCCCCCCC)CCCCCCCC] | heptadecan- 9-yl 9-[(2S)- 4-{[3- (dimethyl- amino) propanoyl] oxy}-1-[6- oxo-6- (undecyloxy) hexyl] pyrrolidine-2- carbonyloxy] nonanoate |
| 2294 | 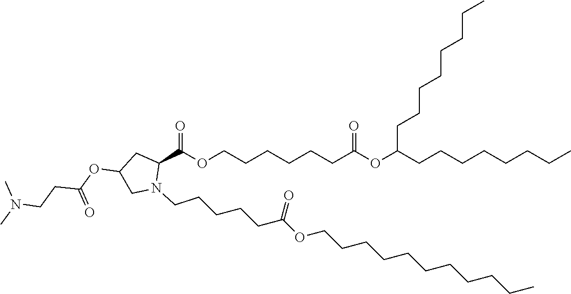 [SMILES: CCCCCCCCCCCOC(=O)CCCCCN1CC(OC(=O)CCN(C)C)C[C@H]1C(=O)OCCCCCCC(=O)OC(CCCCCCCC)CCCCCCCC] | heptadecan- 9-yl 7-[(2S)-4-{[3- (dimethyl- amino) propanoyl] oxy}-1-[6- oxo-6- (undecyloxy) hexyl] pyrrolidine-2- carbonyloxy] heptanoate |
| 2293 | 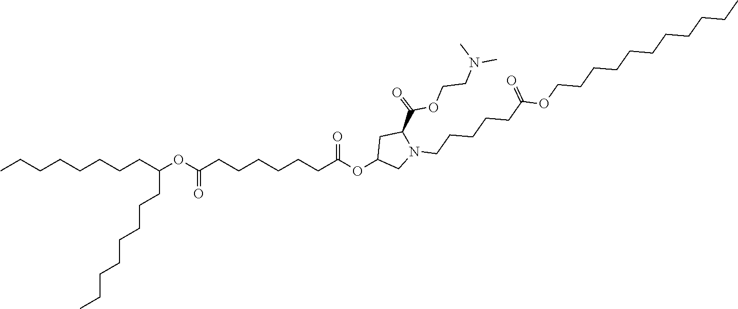 [SMILES: CCCCCCCCCCCOC(=O)CCCCCN1CC(OC(=O)CCCCCCC(=O)OC(CCCCCCCC)CCCCCCCC)C[C@H]1C(=O)OCCN(C)C] | (5S)-5-{[2- (dimethyl- amino) ethoxy] carbonyl}- 1-[6-oxo-6- (undecyloxy) hexyl] pyrrolidin-3-yl 1-heptadecan- 9-yl octanedioate |
| 2292 | 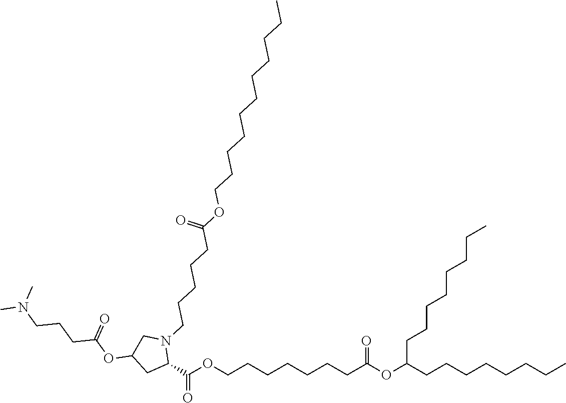 [SMILES: CCCCCCCCCCCOC(=O)CCCCCN1CC(OC(=O)CCCN(C)C)C[C@H]1C(=O)OCCCCCCCC(=O)OC(CCCCCCCC)CCCCCCCC] | heptadecan- 9-yl 8-[(2S)-4-{[4- (dimethyl- amino) butanoyl] oxy}-1-[6- oxo-6- (undecyloxy) hexyl] pyrrolidine-2- carbonyloxy] octanoate |
| 2291 | 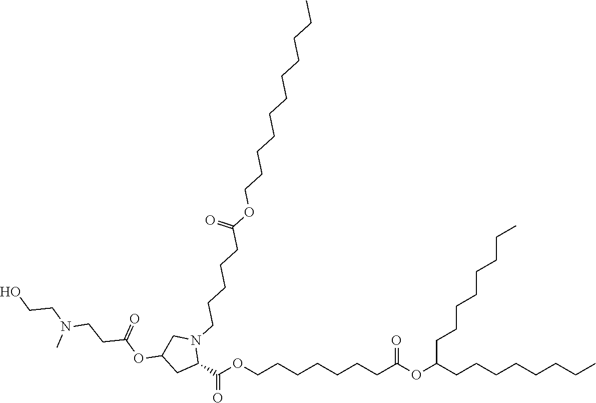 [SMILES: CCCCCCCCCCCOC(=O)CCCCCN1CC(OC(=O)CCN(C)CCO)C[C@H]1C(=O)OCCCCCCCC(=O)OC(CCCCCCCC)CCCCCCCC] | heptadecan- 9-yl 8-[(2S)-4- ({3-[(2- hydroxyethyl) (methyl) amino] propanoyl} oxy)-1-[6- oxo-6- (undecyloxy) hexyl] pyrrolidine-2- carbonyloxy] octanoate |
| 2290 | 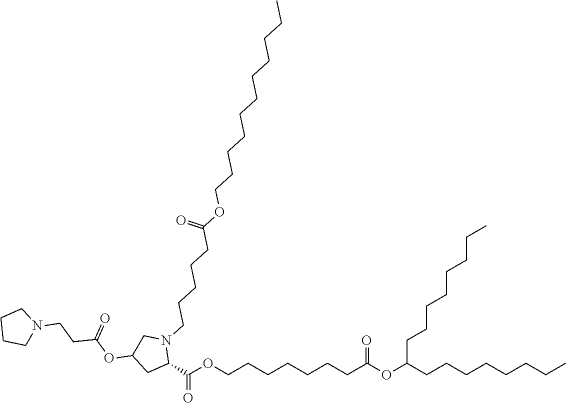 [SMILES: CCCCCCCCCCCOC(=O)CCCCCN1CC(OC(=O)CCN2CCCC2)C[C@H]1C(=O)OCCCCCCCC(=O)OC(CCCCCCCC)CCCCCCCC] | heptadecan- 9-yl 8-[(2S)-1- [6-oxo-6- (undecyloxy) hexyl]-4-{[3- (pyrrolidin- 1-yl) propanoyl] oxy} pyrrolidine-2- carbonyloxy] octanoate |
| 2270 | 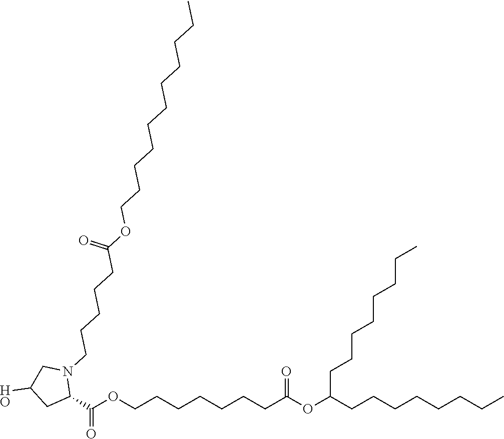 [SMILES: CCCCCCCCCCCOC(=O)CCCCCN1CC(O)C[C@H]1C(=O)OCCCCCCCC(=O)OC(CCCCCCCC)CCCCCCCC] | heptadecan- 9-yl 8-[(2S)-4- hydroxy-1- [6-oxo-6- (undecyloxy) hexyl] pyrrolidine-2- carbonyloxy] octanoate |
| 2260 | 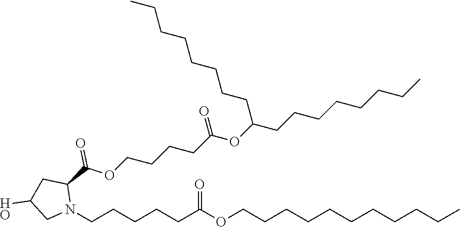 [SMILES: CCCCCCCCCCCOC(=O)CCCCCN1CC(O)C[C@H]1C(=O)OCCCCC(=O)OC(CCCCCCCC)CCCCCCCC] | undecyl 6-[(2S)- 2-({[5- (heptadecan- 9-yloxy)-5- oxopentyl] oxy} carbonyl)-4- hydroxy- pyrrolidin- 1-yl] hexanoate |
| 2240 | 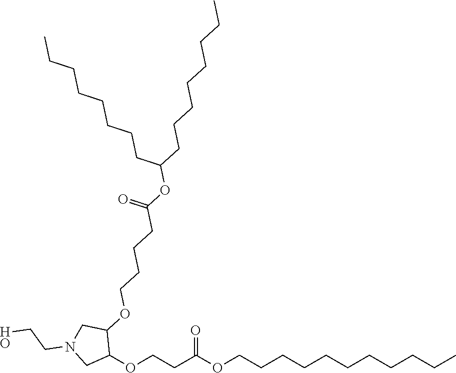 [SMILES: CCCCCCCCCCCOC(=O)CCOC1CN(CCO)CC1OCCCCC(=O)OC(CCCCCCCC)CCCCCCCC] | heptadecan- 9-yl 5-{[1-(2- hydroxyethyl)- 4-[3-oxo-3- (undecyloxy) propoxy] pyrrolidin-3- yl]oxy} pentanoate |
| 2231 | 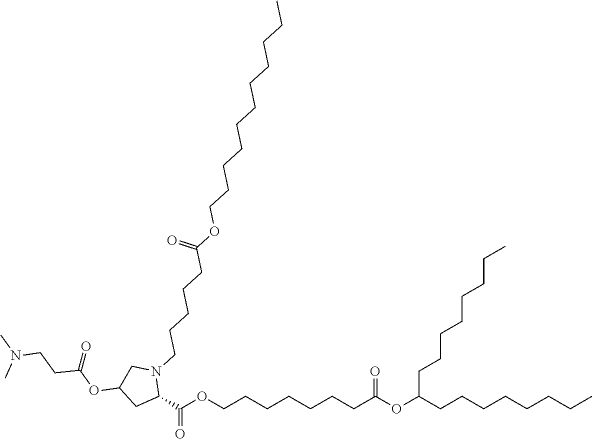 [SMILES: CCCCCCCCCCCOC(=O)CCCCCN1CC(OC(=O)CCN(C)C)C[C@H]1C(=O)OCCCCCCCC(=O)OC(CCCCCCCC)CCCCCCCC] | heptadecan- 9-yl 8-[(2S)-4-{[3- (dimethyl- amino) propanoyl] oxy}-1-[6- oxo-6- (undecyloxy) hexyl] pyrrolidine-2- carbonyloxy] octanoate |
| 2230 | 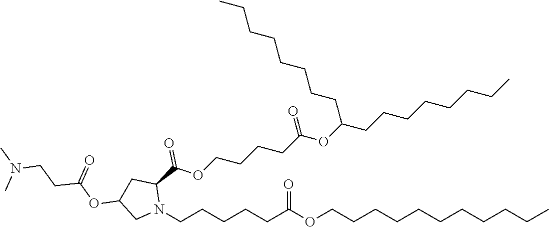 [SMILES: CCCCCCCCCCCOC(=O)CCCCCN1CC(OC(=O)CCN(C)C)C[C@H]1C(=O)OCCCCC(=O)OC(CCCCCCCC)CCCCCCCC] | undecyl 6-[(2S)-4-{[3- (dimethyl- amino) propanoyl] oxy}-2-({[5- (heptadecan- 9-yloxy)-5- oxopentyl] oxy} carbonyl) pyrrolidin- 1-yl] hexanoate |
| 2329 | 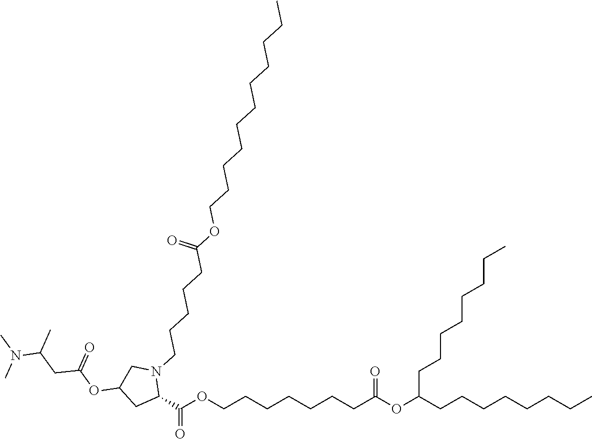 [SMILES: CCCCCCCCCCCOC(=O)CCCCCN1CC(OC(=O)CC(C)N(C)C)C[C@H]1C(=O)OCCCCCCCC(=O)OC(CCCCCCCC)CCCCCCCC] | 8-(heptadecan- 9-yloxy)- 8-oxooctyl (2S)-4-((3- (dimethyl- amino) butanoyl) oxy)-1-(6- oxo-6- (undecyloxy) hexyl) pyrrolidine-2- carboxylate |
| 2336 | 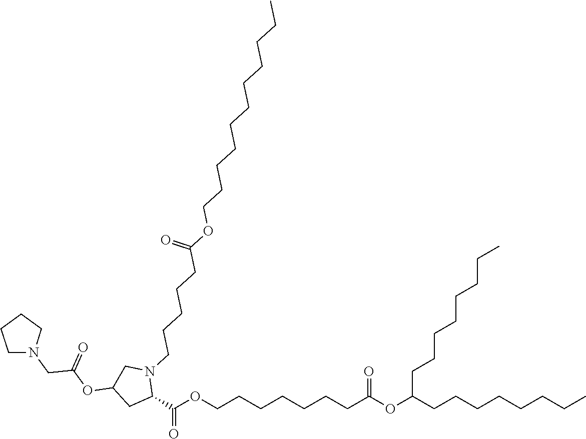 [SMILES: CCCCCCCCCCCOC(=O)CCCCCN1CC(OC(=O)CN2CCCC2)C[C@H]1C(=O)OCCCCCCCC(=O)OC(CCCCCCCC)CCCCCCCC] | 8-(heptadecan- 9-yloxy)- 8-oxooctyl (2S)-1-(6- oxo-6- (undecyloxy) hexyl)-4-(2- (pyrrolidin-1- yl)acetoxy) pyrrolidine- 2-carboxylate |
| 2337 | 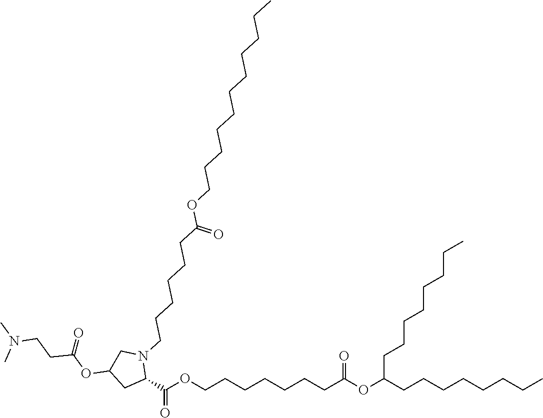 [SMILES: CCCCCCCCCCCOC(=O)CCCCCCN1CC(OC(=O)CCN(C)C)C[C@H]1C(=O)OCCCCCCCC(=O)OC(CCCCCCCC)CCCCCCCC] | 8-(heptadecan- 9-yloxy)- 8-oxooctyl (2S)-4-((3- (dimethyl- amino) propanoyl) oxy)-1- (7-oxo-7- (undecyloxy) heptyl) pyrrolidine-2- carboxylate |
| 2338 | 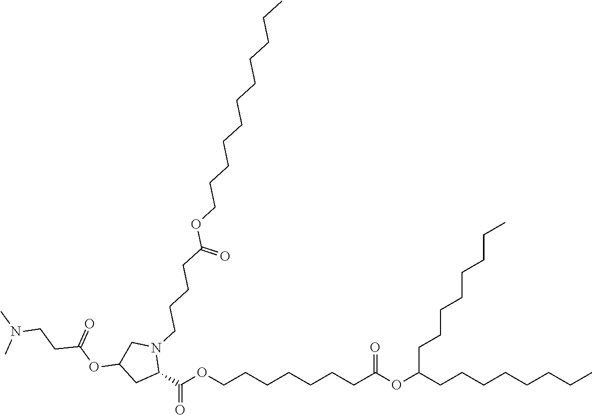 [SMILES: CCCCCCCCCCCOC(=O)CCCCN1CC(OC(=O)CCN(C)C)C[C@H]1C(=O)OCCCCCCCC(=O)OC(CCCCCCCC)CCCCCCCC] | 8-(heptadecan- 9-yloxy)- 8-oxooctyl (2S)-4-((3- (dimethyl- amino) propanoyl) oxy)-1- (5-oxo-5- (undecyloxy) pentyl) pyrrolidine-2- carboxylate |
| 2339 | 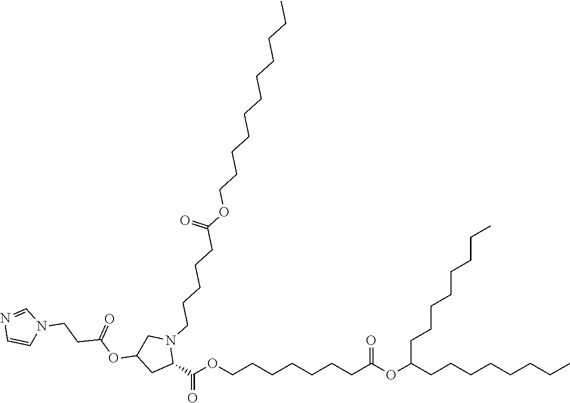 [SMILES: CCCCCCCCCCCOC(=O)CCCCCN1CC(OC(=O)CCn2ccnc2)C[C@H]1C(=O)OCCCCCCCC(=O)OC(CCCCCCCC)CCCCCCCC] | 8-(heptadecan- 9-yloxy)- 8-oxooctyl (2S)-4-((3- (1H-imidazol- 1-yl) propanoyl) oxy)-1-(6- oxo-6- (undecyloxy) hexyl) pyrrolidine-2- carboxylate |
| 2340 | 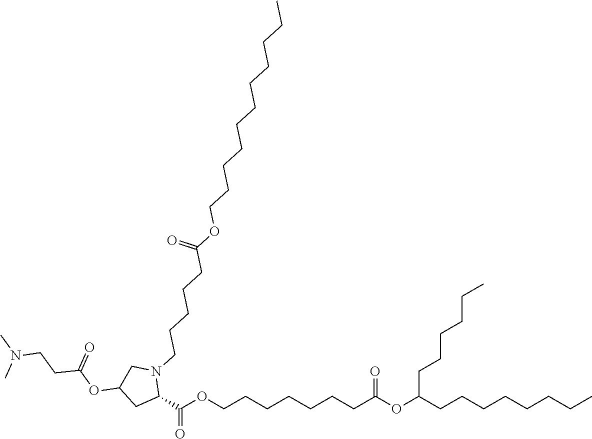 [SMILES: CCCCCCCCCCCOC(=O)CCCCCN1CC(OC(=O)CCN(C)C)C[C@H]1C(=O)OCCCCCCCC(=O)OC(CCCCCC)CCCCCCCC] | 8-oxo-8- (pentadecan-7- yloxy)octyl (2S)-4-((3- (dimethyl- amino) propanoyl) oxy)-1- (6-oxo-6- (undecyloxy) hexyl) pyrrolidine-2- carboxylate |
| 2341 | 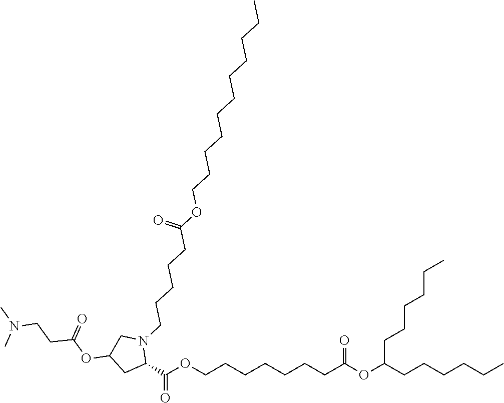 [SMILES: CCCCCCCCCCCOC(=O)CCCCCN1CC(OC(=O)CCN(C)C)C[C@H]1C(=O)OCCCCCCCC(=O)OC(CCCCCC)CCCCCC] | 8-oxo-8- (tridecan-7- yloxy)octyl (2S)-4-((3- (dimethyl- amino) propanoyl) oxy)-1- (6-oxo-6- (undecyloxy) hexyl) pyrrolidine-2- carboxylate |
| 2342 | 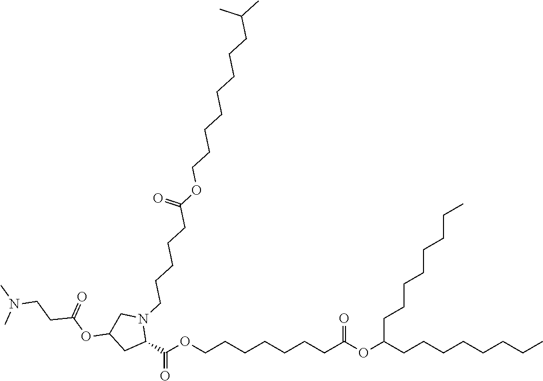 [SMILES: CCCCCCCCC(CCCCCCCC)OC(=O)CCCCCCCOC(=O)[C@@H]1CC(OC(=O)CCN(C)C)CN1CCCCCC(=O)OCCCCCCCCC(C)C] | 8-(heptadecan- 9-yloxy)- 8-oxooctyl (2S)-4-((3- (dimethyl- amino) propanoyl) oxy)-1-(6-((9- methyldecyl) oxy)-6- oxohexyl) pyrrolidine-2- carboxylate |
| 2343 | 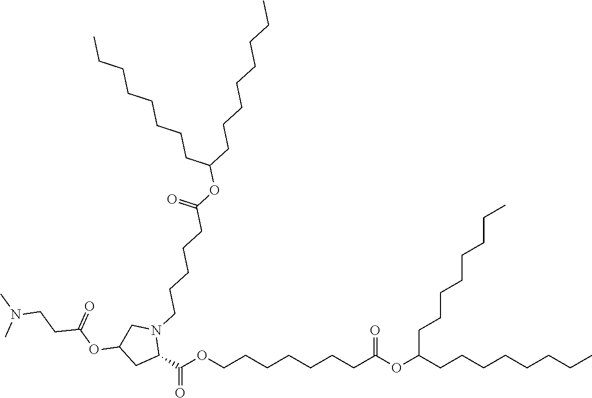 [SMILES: CCCCCCCCC(CCCCCCCC)OC(=O)CCCCCCCOC(=O)[C@@H]1CC(OC(=O)CCN(C)C)CN1CCCCCC(=O)OC(CCCCCCCC)CCCCCCCC] | 8-(heptadecan- 9-yloxy)- 8-oxooctyl (2S)-4-((3- (dimethyl- amino) propanoyl) oxy)-1-(6- (heptadecan- 9-yloxy)-6- oxohexyl) pyrrolidine-2- carboxylate |
| 2344 | 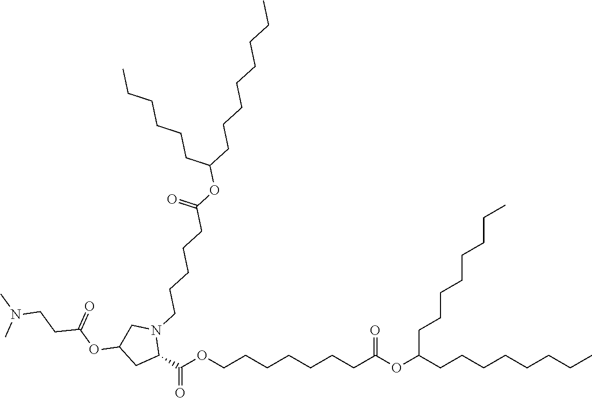 [SMILES: CCCCCCCCC(CCCCCC)OC(=O)CCCCCN1CC(OC(=O)CCN(C)C)C[C@H]1C(=O)OCCCCCCCC(=O)OC(CCCCCCCC)CCCCCCCC] | 8-(heptadecan- 9-yloxy)- 8-oxooctyl (2S)-4-((3- (dimethyl- amino) propanoyl) oxy)-1- (6-oxo-6- (pentadecan-7- yloxy)hexyl) pyrrolidine- 2-carboxylate |
| 2345 | 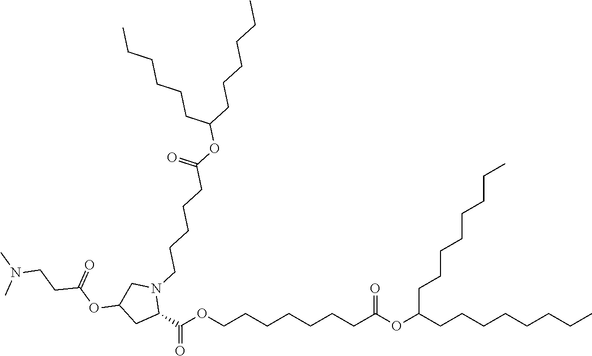 [SMILES: CCCCCCCCC(CCCCCCCC)OC(=O)CCCCCCCOC(=O)[C@@H]1CC(OC(=O)CCN(C)C)CN1CCCCCC(=O)OC(CCCCCC)CCCCCC] | 8-(heptadecan- 9-yloxy)- 8-oxooctyl (2S)-4-((3- (dimethyl- amino) propanoyl) oxy)-1- (6-oxo-6- (tridecan-7- yloxy)hexyl) pyrrolidine- 2-carboxylate |
| 2348 | 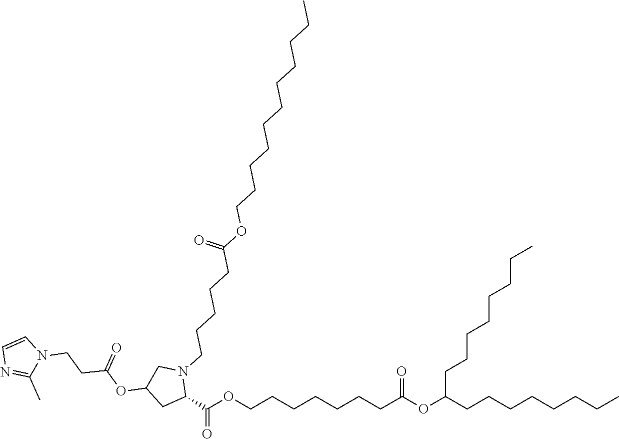 [SMILES: CCCCCCCCCCCOC(=O)CCCCCN1CC(OC(=O)CCn2ccnc2C)C[C@H]1C(=O)OCCCCCCCC(=O)OC(CCCCCCCC)CCCCCCCC] | 8-(heptadecan- 9-yloxy)- 8-oxooctyl (2S)-4-((3- (2-methyl-1H- imidazol-1-yl) propanoyl) oxy)- 1-(6-oxo-6- (undecyloxy) hexyl) pyrrolidine-2- carboxylate |
| 2349 | 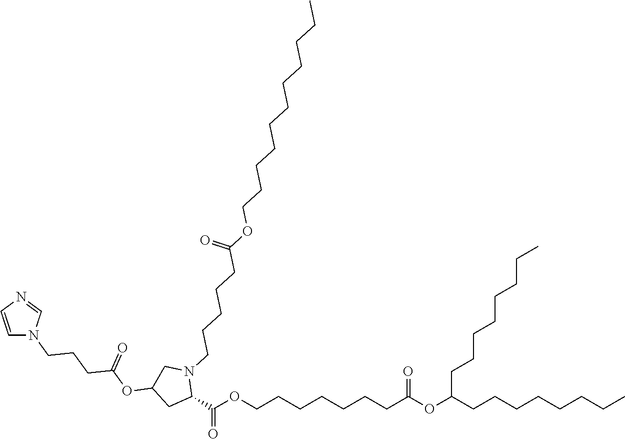 [SMILES: CCCCCCCCCCCOC(=O)CCCCCN1CC(OC(=O)CCCn2ccnc2)C[C@H]1C(=O)OCCCCCCCC(=O)OC(CCCCCCCC)CCCCCCCC] | 8-(heptadecan- 9-yloxy)- 8-oxooctyl (2S)-4-((4- (1H- imidazol-1- yl)butanoyl) oxy)-1-(6- oxo-6- (undecyloxy) hexyl) pyrrolidine-2- carboxylate |
| 2352 | 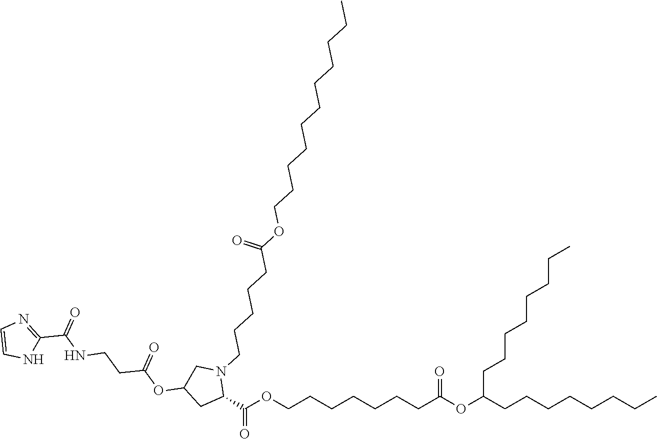 [SMILES: CCCCCCCCCCCOC(=O)CCCCCN1CC(OC(=O)CCNC(=O)c2ncc[nH]2)C[C@H]1C(=O)OCCCCCCCC(=O)OC(CCCCCCCC)CCCCCCCC] | 8-(heptadecan- 9-yloxy)- 8-oxooctyl (2S)-4-((3- (1H- imidazole-2- carboxamido) propanoyl) oxy)- 1-(6-oxo-6- (undecyloxy) hexyl) pyrrolidine-2- carboxylate |
| 2371 | 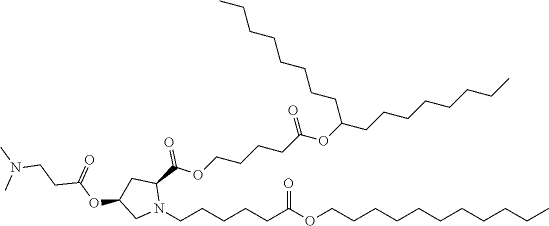 [SMILES: CCCCCCCCCCCOC(=O)CCCCCN1C[C@@H](OC(=O)CCN(C)C)C[C@H]1C(=O)OCCCCC(=O)OC(CCCCCCCC)CCCCCCCC] | 5-(heptadecan- 9-yloxy)- 5-oxopentyl (2S,4S)-4-((3- (dimethyl- amino) propanoyl) oxy)-1- (6-oxo-6- (undecyloxy) hexyl) pyrrolidine-2- carboxylate |
| 2372 | 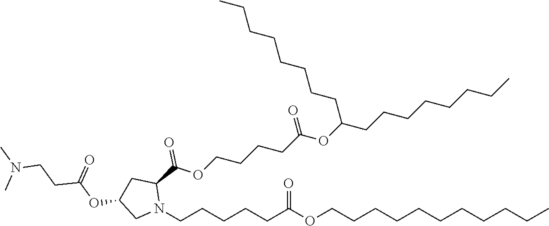 [SMILES: CCCCCCCCCCCOC(=O)CCCCCN1C[C@H](OC(=O)CCN(C)C)C[C@H]1C(=O)OCCCCC(=O)OC(CCCCCCCC)CCCCCCCC] | 5-(heptadecan- 9-yloxy)- 5-oxopentyl (2S,4R)-4-((3- (dimethyl- amino) propanoyl) oxy)-1- (6-oxo-6- (undecyloxy) hexyl) pyrrolidine-2- carboxylate |
| 2373 | 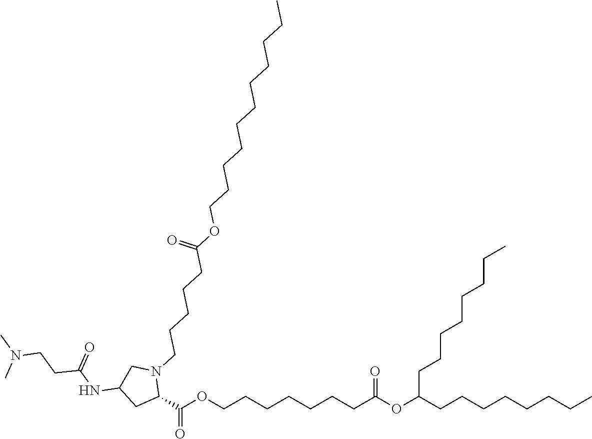 [SMILES: CCCCCCCCCCCOC(=O)CCCCCN1CC(NC(=O)CCN(C)C)C[C@H]1C(=O)OCCCCCCCC(=O)OC(CCCCCCCC)CCCCCCCC] | 8-(heptadecan- 9-yloxy)- 8-oxooctyl (2S)-4-(3- (dimethyl- amino) propanamido)- 1-(6-oxo-6- (undecyloxy) hexyl) pyrrolidine-2- carboxylate |
| 2375 | 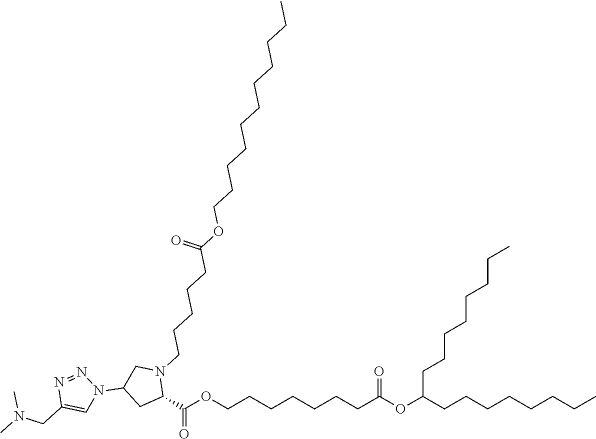 [SMILES: CCCCCCCCCCCOC(=O)CCCCCN1CC(n2cc(CN(C)C)nn2)C[C@H]1C(=O)OCCCCCCCC(=O)OC(CCCCCCCC)CCCCCCCC] | 8-(heptadecan- 9-yloxy)- 8-oxooctyl (2S)-4-(4- ((dimethyl- amino) methyl)- 1H-1,2,3- triazol-1- yl)-1-(6-oxo-6- (undecyloxy) hexyl) pyrrolidine-2- carboxylate |
| 2376 | 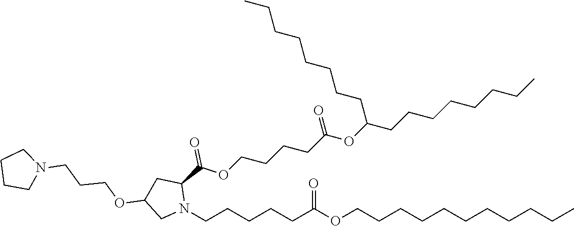 [SMILES: CCCCCCCCCCCOC(=O)CCCCCN1CC(OCCCN2CCCC2)C[C@H]1C(=O)OCCCCC(=O)OC(CCCCCCCC)CCCCCCCC] | 5-(heptadecan- 9-yloxy)-5- oxopentyl (2S)-1- (6-oxo-6- (undecyloxy) hexyl)-4-(3- (pyrrolidin-1- yl)propoxy) pyrrolidine- 2-carboxylate |
| 2377 | 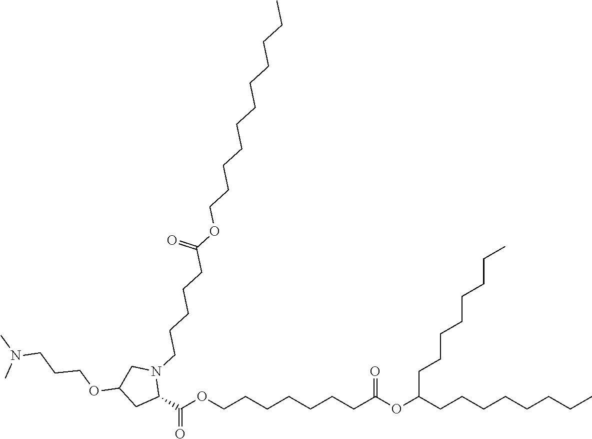 [SMILES: CCCCCCCCCCCOC(=O)CCCCCN1CC(OCCCN(C)C)C[C@H]1C(=O)OCCCCCCCC(=O)OC(CCCCCCCC)CCCCCCCC] | 8-(heptadecan- 9-yloxy)- 8-oxooctyl (2S)-4-(3- (dimethyl- amino) propoxy)-1- (6-oxo-6- (undecyloxy) hexyl) pyrrolidine-2- carboxylate |
| 2436 | 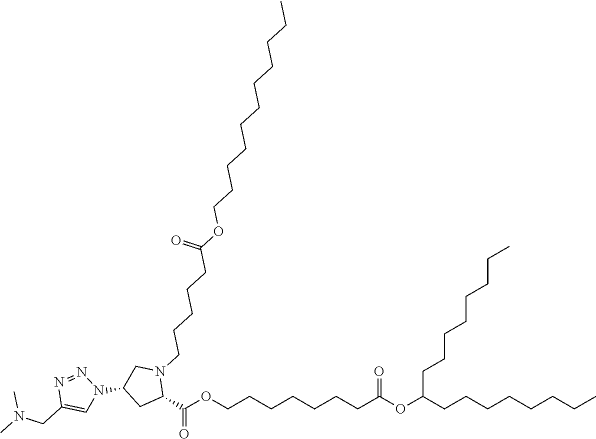 [SMILES: CCCCCCCCCCCOC(=O)CCCCCN1C[C@@H](n2cc(CN(C)C)nn2)C[C@H]1C(=O)OCCCCCCCC(=O)OC(CCCCCCCC)CCCCCCCC] | 8-(heptadecan- 9-yloxy)-8- oxooctyl (2S,4S)-4-(4- ((dimethyl- amino) methyl)- 1H-1,2,3- triazol-1-yl)- 1-(6-oxo-6- (undecyloxy) hexyl) pyrrolidine- 2-carboxylate |
| 2437 | 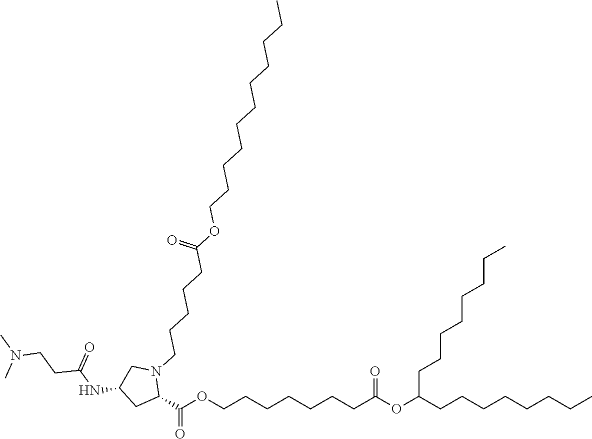 [SMILES: CCCCCCCCCCCOC(=O)CCCCCN1C[C@@H](NC(=O)CCN(C)C)C[C@H]1C(=O)OCCCCCCCC(=O)OC(CCCCCCCC)CCCCCCCC] | 8-(heptadecan- 9-yloxy)- 8-oxooctyl (2S,4S)-4-(3- (dimethyl- amino) propanamido)- 1-(6-oxo-6- (undecyloxy) hexyl) pyrrolidine-2- carboxylate |
| 2440 | 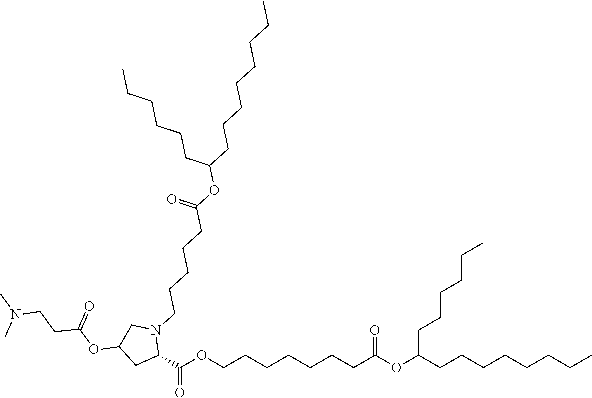 [SMILES: CCCCCCCCC(CCCCCC)OC(=O)CCCCCCCOC(=O)[C@@H]1CC(OC(=O)CCN(C)C)CN1CCCCCC(=O)OC(CCCCCC)CCCCCCCC] | 8-oxo-8- (pentadecan-7- yloxy)octyl (2S)-4-((3- (dimethyl- amino) propanoyl) oxy)- 1-(6-oxo-6- (pentadecan-7- yloxy)hexyl) pyrrolidine- 2-carboxylate |
| 2452 | 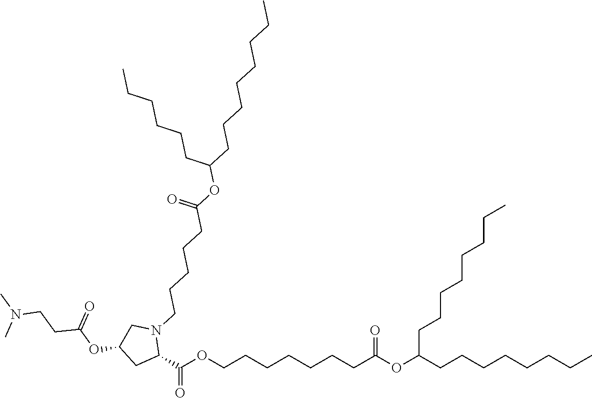 [SMILES: CCCCCCCCC(CCCCCC)OC(=O)CCCCCN1C[C@@H](OC(=O)CCN(C)C)C[C@H]1C(=O)OCCCCCCCC(=O)OC(CCCCCCCC)CCCCCCCC] | 8-(heptadecan- 9-yloxy)- 8-oxooctyl (2S,4S)-4-((3- (dimethyl- amino) propanoyl) oxy)- 1-(6-oxo-6- (pentadecan-7- yloxy)hexyl) pyrrolidine- 2-carboxylate |

Additional non-limiting examples of ionizable lipid compounds disclosed here are set forth in

TABLE 2
Exemplary ionizable lipid compounds.

| | | |
|---|---|---|
| 2232 | 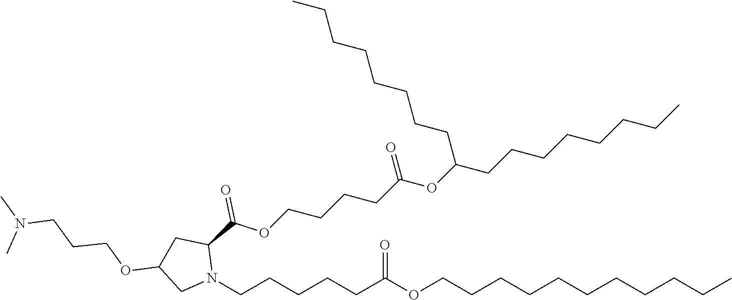 [SMILES: CCCCCCCCCCCOC(=O)CCCCCN1CC(OCCCN(C)C)C[C@H]1C(=O)OCCCCC(=O)OC(CCCCCCCC)CCCCCCCC] | undecyl 6-[(2S)-4-[3- (dimethylamino)propoxy]- 2-({[5-(heptadecan-9- yloxy)-5- oxopentyl]oxy}carbonyl) pyrrolidin-1-yl]hexanoate |
| 2350 | 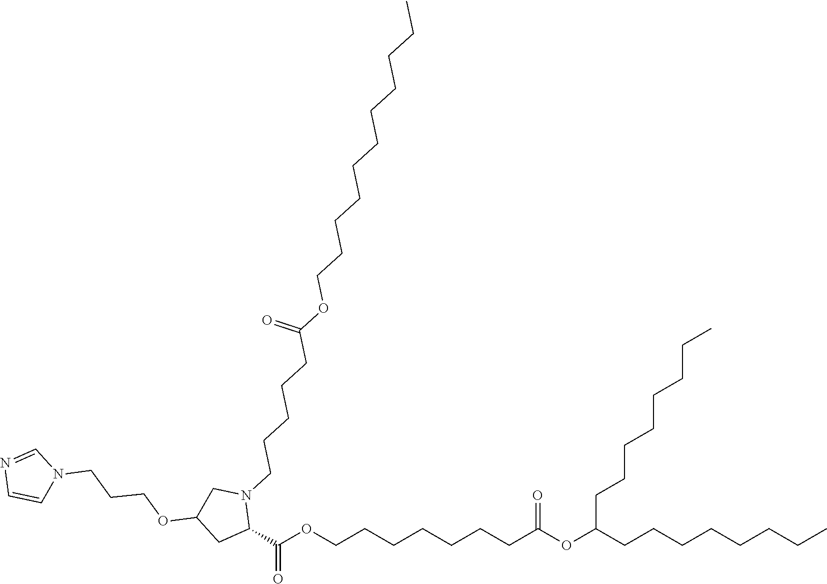 [SMILES: CCCCCCCCCCCOC(=O)CCCCCN1CC(OCCCn2ccnc2)C[C@H]1C(=O)OCCCCCCCC(=O)OC(CCCCCCCC)CCCCCCCC] | 8-(heptadecan-9-yloxy)-8- oxooctyl (2S)-4-(3-(1H- imidazol-1-yl)propoxy)-1- (6-oxo-6- (undecyloxy)hexyl) pyrrolidine-2-carboxylate |
| 2351 | 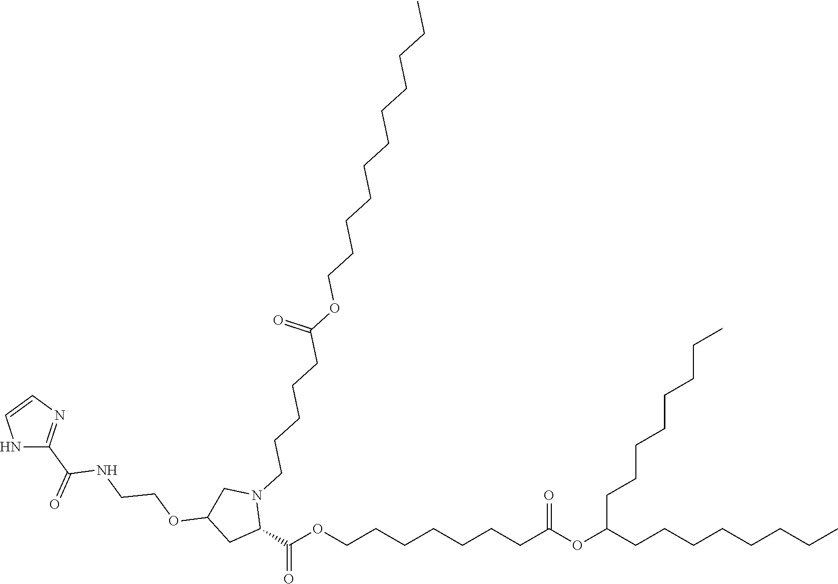 [SMILES: CCCCCCCCCCCOC(=O)CCCCCN1CC(OCCNC(=O)c2ncc[nH]2)C[C@H]1C(=O)OCCCCCCCC(=O)OC(CCCCCCCC)CCCCCCCC] | 8-(heptadecan-9-yloxy)-8- oxooctyl (2S)-4-(2-(1H- imidazole-2- carboxamido)ethoxy)-1- (6-oxo-6- (undecyloxy)hexyl) pyrrolidine-2-carboxylate |
| 2359 | 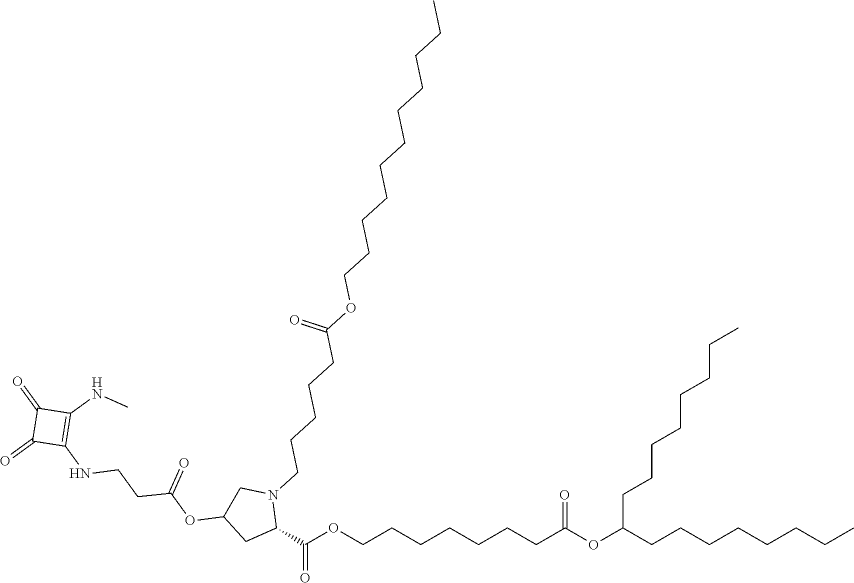 [SMILES: CCCCCCCCCCCOC(=O)CCCCCN1CC(OC(=O)CCNc2c(NC)c(=O)c2=O)C[C@H]1C(=O)OCCCCCCCC(=O)OC(CCCCCCCC)CCCCCCCC] | 8-(heptadecan-9-yloxy)-8- oxooctyl (2S)-4-((3-((2- (methylamino)-3,4- dioxocyclobut-1-en-1- yl)amino)propanoyl)oxy)- 1-(6-oxo-6- (undecyloxy)hexyl) pyrrolidine-2-carboxylate |
| 2360 | 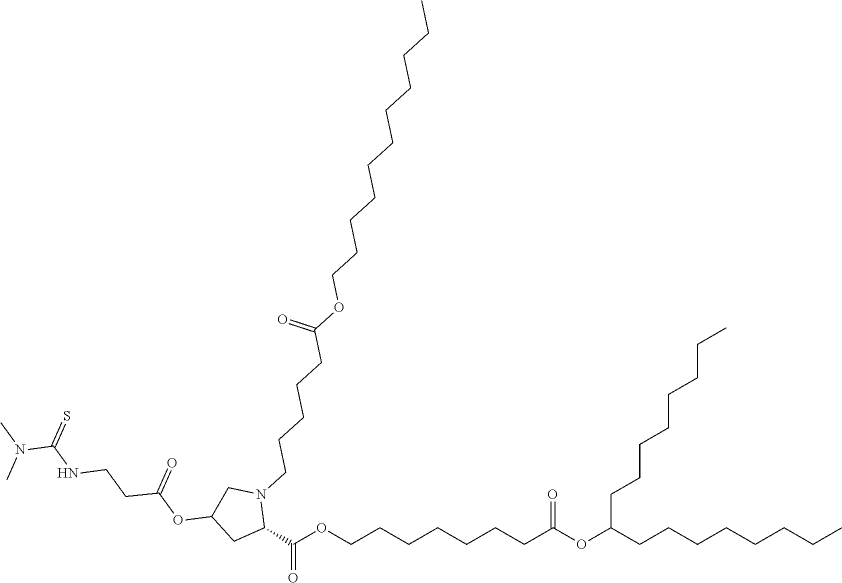 [SMILES: CCCCCCCCCCCOC(=O)CCCCCN1CC(OC(=O)CCNC(=S)N(C)C)C[C@H]1C(=O)OCCCCCCCC(=O)OC(CCCCCCCC)CCCCCCCC] | 8-(heptadecan-9-yloxy)-8- oxooctyl (2S)-4-((3-(3,3- dimethylthioureido) propanoyl)oxy)-1-(6-oxo-6- (undecyloxy)hexyl) pyrrolidine-2-carboxylate |
| 2363 | 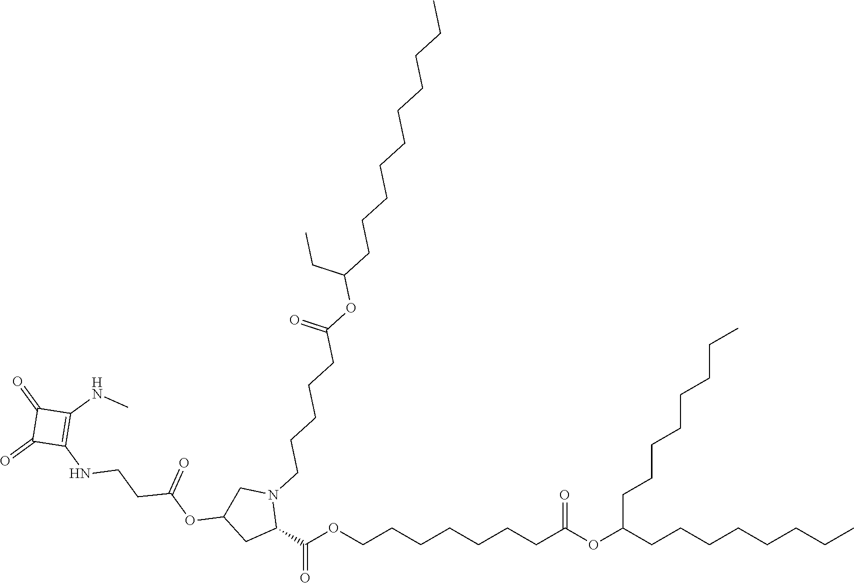 [SMILES: CCCCCCCCCCC(CC)OC(=O)CCCCCN1CC(OC(=O)CCNc2c(NC)c(=O)c2=O)C[C@H]1C(=O)OCCCCCCCC(=O)OC(CCCCCCCC)CCCCCCCC] | 8-(heptadecan-9-yloxy)-8- oxooctyl (2S)-4-((3-((2- (methylamino)-3,4- dioxocyclobut-1-en-1- yl)amino)propanoyl)oxy)- 1-(6-oxo-6-(tridecan-3- yloxy)hexyl)pyrrolidine-2- carboxylate |
| 2364 | 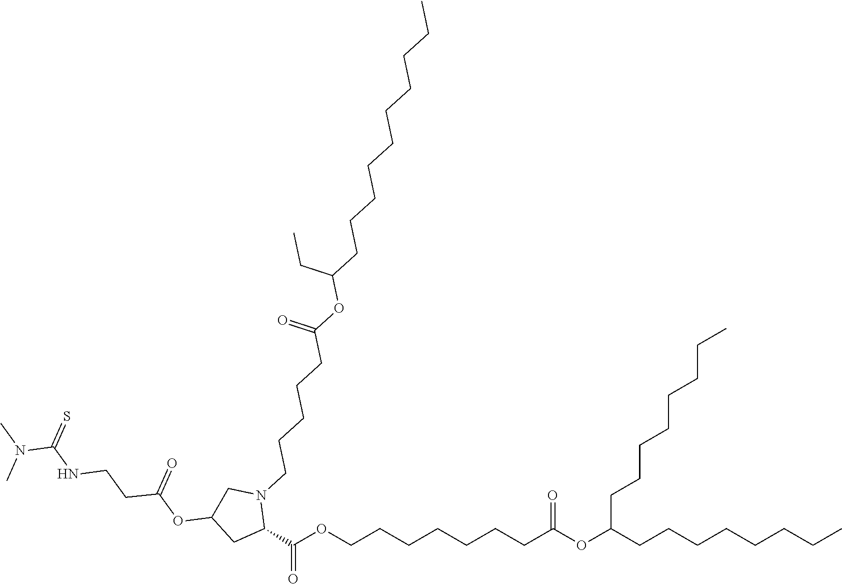 [SMILES: CCCCCCCCCCC(CC)OC(=O)CCCCCN1CC(OC(=O)CCNC(=S)N(C)C)C[C@H]1C(=O)OCCCCCCCC(=O)OC(CCCCCCCC)CCCCCCCC] | 8-(heptadecan-9-yloxy)-8- oxooctyl (2S)-4-((3-(3,3- dimethylthioureido) propanoyl)oxy)-1-(6-oxo-6- (tridecan-3- yloxy)hexyl)pyrrolidine-2- carboxylate |
| 2374 | 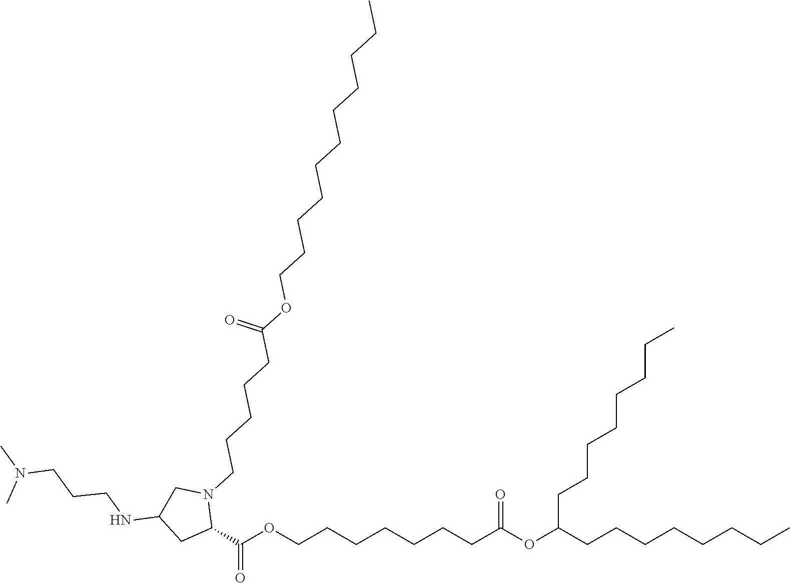 [SMILES: CCCCCCCCCCCOC(=O)CCCCCN1CC(NCCCN(C)C)C[C@H]1C(=O)OCCCCCCCC(=O)OC(CCCCCCCC)CCCCCCCC] | 8-(heptadecan-9-yloxy)-8- oxooctyl (2S)-4-((3- (dimethylamino)propyl) amino)-1-(6-oxo-6- (undecyloxy)hexyl) pyrrolidine-2-carboxylate |
| 2422 | 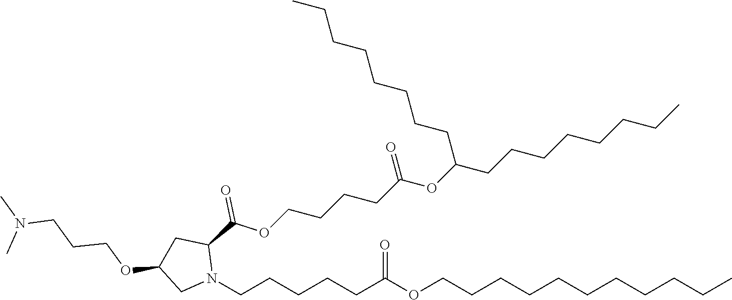 [SMILES: CCCCCCCCCCCOC(=O)CCCCCN1C[C@@H](OCCCN(C)C)C[C@H]1C(=O)OCCCCC(=O)OC(CCCCCCCC)CCCCCCCC] | 5-(heptadecan-9-yloxy)- 5-oxopentyl (2S,4S)-4- (3- (dimethylamino)propoxy)- 1-(6-oxo-6- (undecyloxy)hexyl) pyrrolidine-2-carboxylate |
| 2423 | 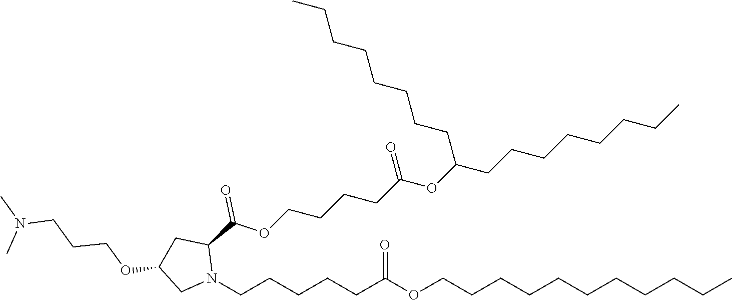 [SMILES: CCCCCCCCCCCOC(=O)CCCCCN1C[C@H](OCCCN(C)C)C[C@H]1C(=O)OCCCCC(=O)OC(CCCCCCCC)CCCCCCCC] | 5-(heptadecan-9-yloxy)-5- oxopentyl (2S,4R)-4-(3- (dimethylamino)propoxy)- 1-(6-oxo-6- (undecyloxy)hexyl) pyrrolidine-2-carboxylate |
| 2429 | 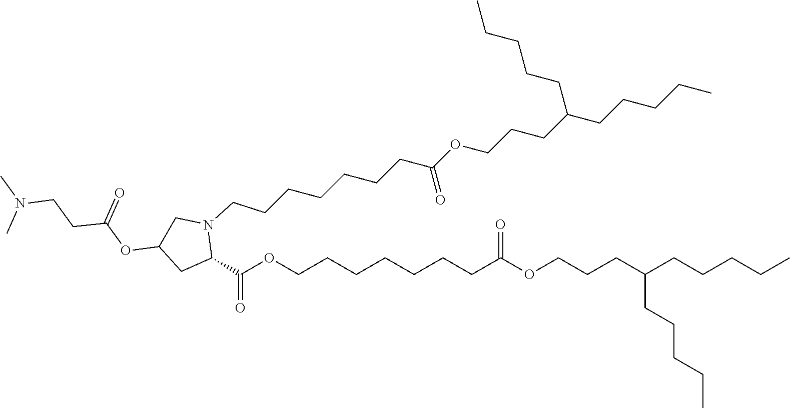 [SMILES: CCCCCC(CCCCC)CCCOC(=O)CCCCCCCOC(=O)[C@@H]1CC(OC(=O)CCN(C)C)CN1CCCCCCCC(=O)OCCCC(CCCCC)CCCCC] | 8-oxo-8-((4- pentylnonyl)oxy)octyl (2S)-4-((3- (dimethylamino)propanoyl) oxy)-1-(8-oxo-8-((4- pentylnonyl)oxy)octyl) pyrrolidine-2-carboxylate |
| 2430 | 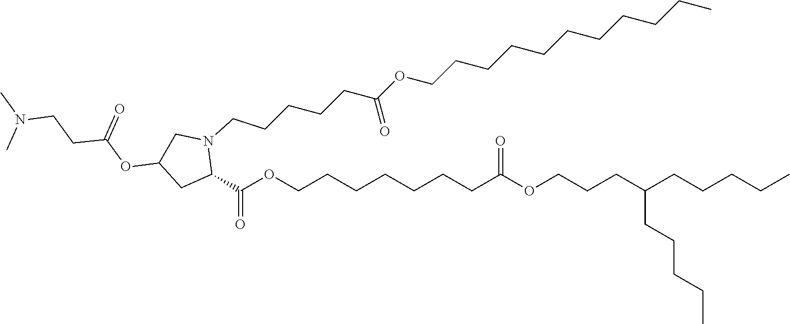 [SMILES: CCCCCCCCCCCOC(=O)CCCCCN1CC(OC(=O)CCN(C)C)C[C@H]1C(=O)OCCCCCCCC(=O)OCCCC(CCCCC)CCCCC] | 8-oxo-8-((4- pentylnonyl)oxy)octyl (2S)-4-((3- (dimethylamino)propanoyl) oxy)-1-(6-oxo-6- (undecyloxy)hexyl) pyrrolidine-2-carboxylate |
| 2431 | 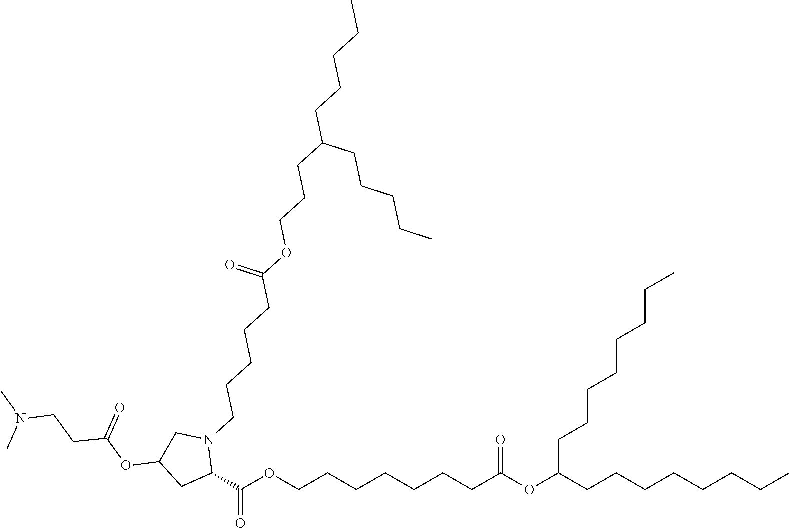 [SMILES: CCCCCCCCC(CCCCCCCC)OC(=O)CCCCCCCOC(=O)[C@@H]1CC(OC(=O)CCN(C)C)CN1CCCCCC(=O)OCCCC(CCCCC)CCCCC] | 8-(heptadecan-9-yloxy)-8- oxooctyl (2S)-4-((3- (dimethylamino)propanoyl) oxy)-1-(6-oxo-6-((4- pentylnonyl)oxy)hexyl) pyrrolidine-2-carboxylate |
| 2438 | 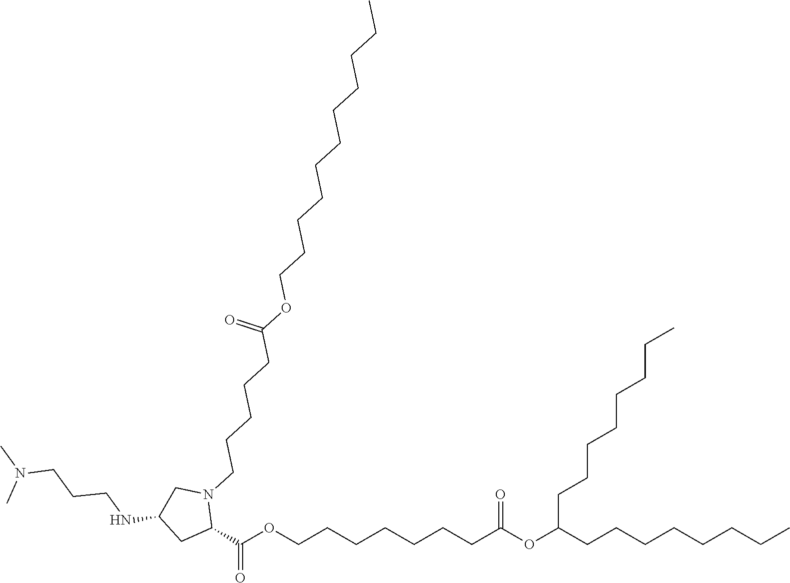 [SMILES: CCCCCCCCCCCOC(=O)CCCCCN1C[C@@H](NCCCN(C)C)C[C@H]1C(=O)OCCCCCCCC(=O)OC(CCCCCCCC)CCCCCCCC] | 8-(heptadecan-9-yloxy)-8- oxooctyl (2S,4S)-4-((3- (dimethylamino)propyl) amino)-1-(6-oxo-6- (undecyloxy)hexyl) pyrrolidine-2-carboxylate |
| 2451 | 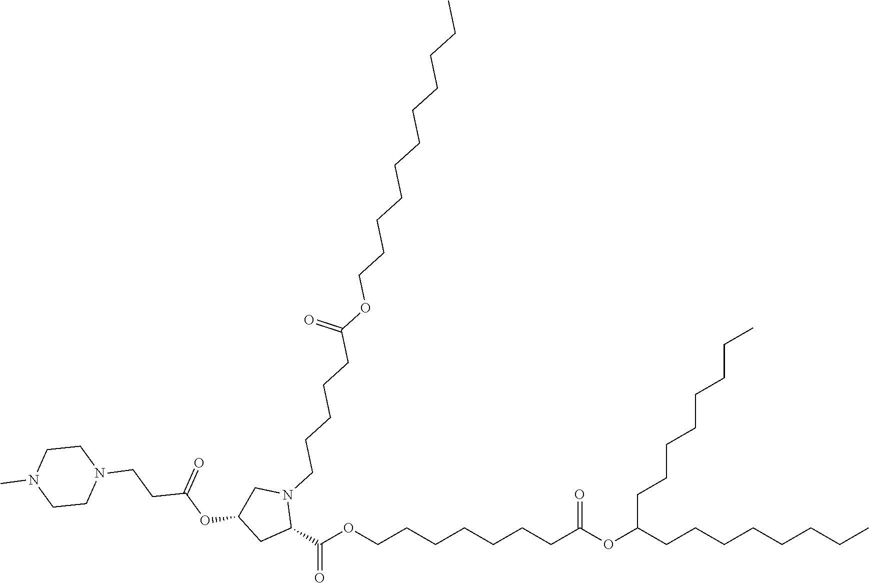 [SMILES: CCCCCCCCCCCOC(=O)CCCCCN1C[C@@H](OC(=O)CCN2CCN(C)CC2)C[C@H]1C(=O)OCCCCCCCC(=O)OC(CCCCCCCC)CCCCCCCC] | 8-(heptadecan-9-yloxy)-8- oxooctyl (2S,4S)-4-((3-(4- methylpiperazin-1- yl)propanoyl)oxy)-1-(6- oxo-6- (undecyloxy)hexyl) pyrrolidine-2-carboxylate |
| 2455 | 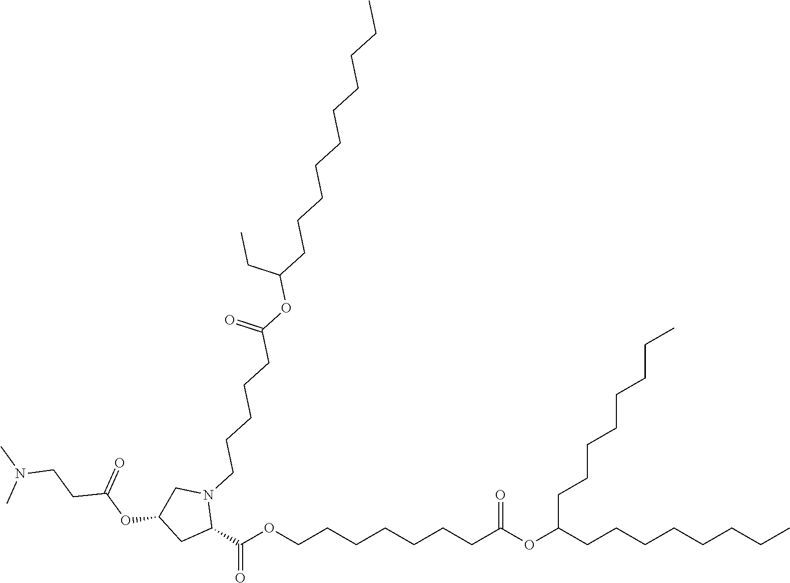 [SMILES: CCCCCCCCCCC(CC)OC(=O)CCCCCN1C[C@@H](OC(=O)CCN(C)C)C[C@H]1C(=O)OCCCCCCCC(=O)OC(CCCCCCCC)CCCCCCCC] | 8-(heptadecan-9-yloxy)-8- oxooctyl (2S,4S)-4-((3- (dimethylamino)propanoyl) oxy)-1-(6-oxo-6- (tridecan-3- yloxy)hexyl)pyrrolidine-2- carboxylate |
| 1 | 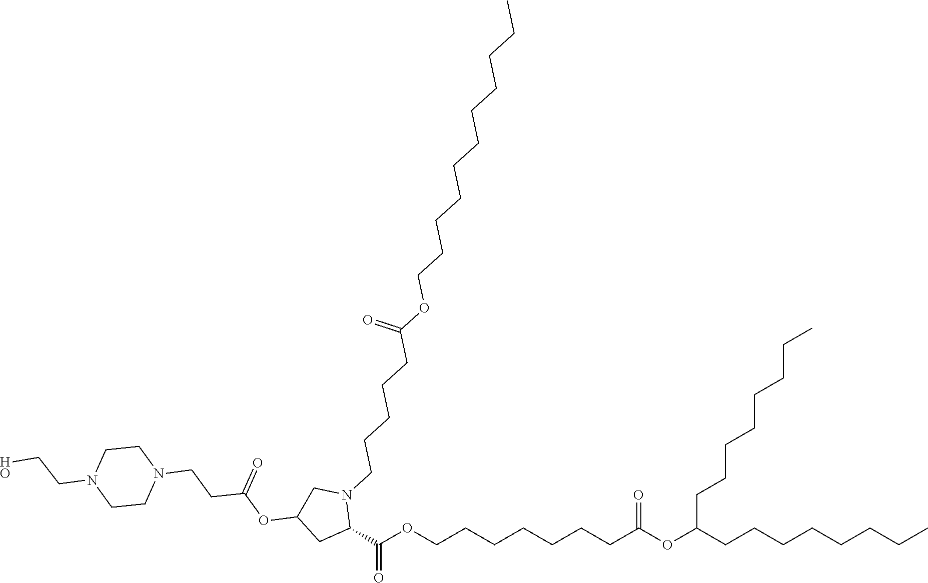 [SMILES: CCCCCCCCCCCOC(=O)CCCCCN1CC(OC(=O)CCN2CCN(CCO)CC2)C[C@H]1C(=O)OCCCCCCCC(=O)OC(CCCCCCCC)CCCCCCCC] | 8-(heptadecan-9-yloxy)- 8-oxooctyl (2S)-4-((3-(4- (2- hydroxyethyl)piperazin- 1-yl)propanoyl)oxy)-1- (6-oxo-6- (undecyloxy)hexyl) pyrrolidine-2-carboxylate |
| 2 | 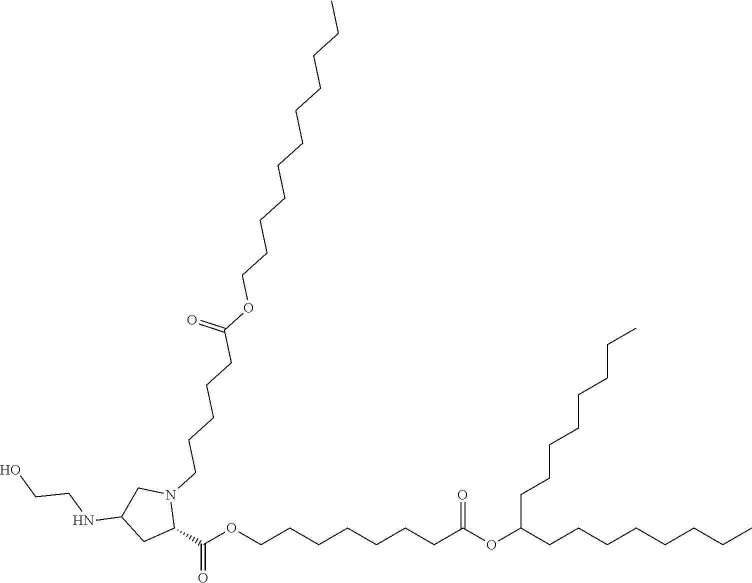 [SMILES: CCCCCCCCCCCOC(=O)CCCCCN1CC(NCCO)C[C@H]1C(=O)OCCCCCCCC(=O)OC(CCCCCCCC)CCCCCCCC] | 8-(heptadecan-9-yloxy)- 8-oxooctyl (2S)-4-((2- hydroxyethyl)amino)-1- (6-oxo-6- (undecyloxy)hexyl) pyrrolidine-2-carboxylate |
| 3 | 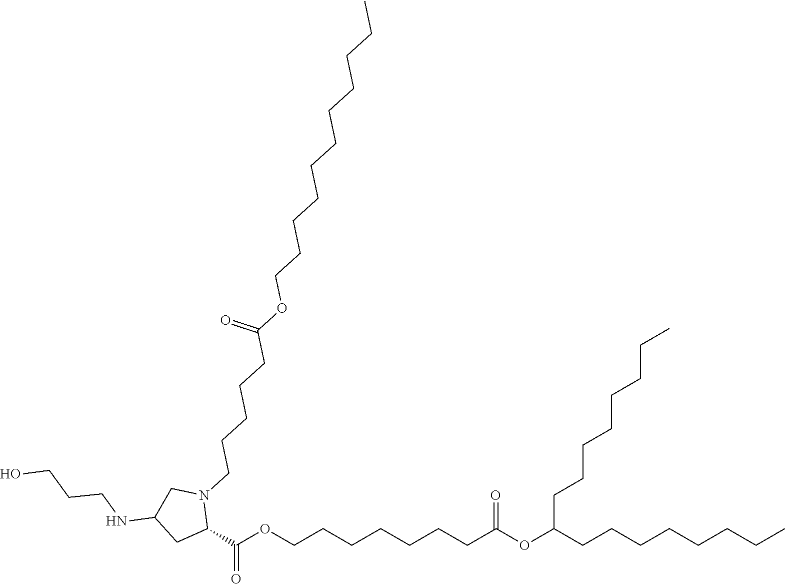 [SMILES: CCCCCCCCCCCOC(=O)CCCCCN1CC(NCCCO)C[C@H]1C(=O)OCCCCCCCC(=O)OC(CCCCCCCC)CCCCCCCC] | 8-(heptadecan-9-yloxy)- 8-oxooctyl (2S)-4-((3- hydroxypropyl)amino)-1- (6-oxo-6- (undecyloxy)hexyl) pyrrolidine-2-carboxylate |
| 4 | 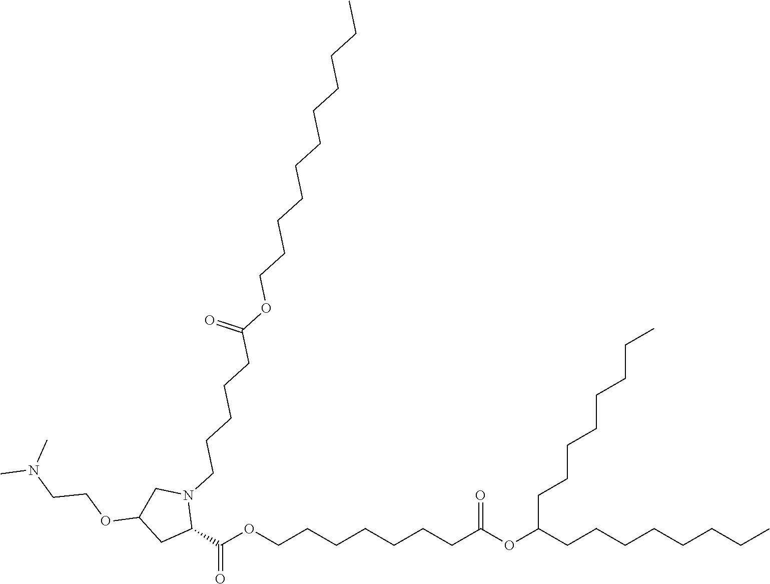 [SMILES: CCCCCCCCCCCOC(=O)CCCCCN1CC(OCCN(C)C)C[C@H]1C(=O)OCCCCCCCC(=O)OC(CCCCCCCC)CCCCCCCC] | 8-(heptadecan-9-yloxy)- 8-oxooctyl (2S)-4-(2- (dimethylamino)ethoxy)- 1-(6-oxo-6- (undecyloxy)hexyl) pyrrolidine-2-carboxylate |
| 5 | 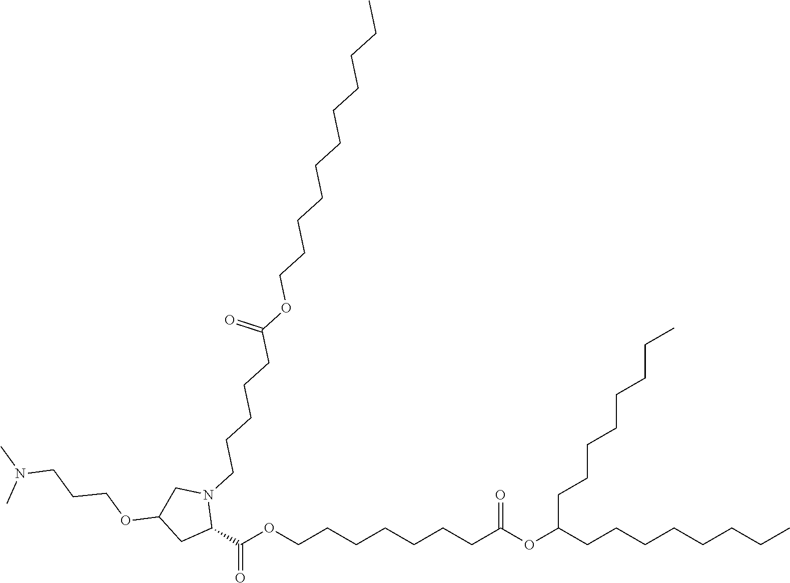 [SMILES: CCCCCCCCCCCOC(=O)CCCCCN1CC(OCCCN(C)C)C[C@H]1C(=O)OCCCCCCCC(=O)OC(CCCCCCCC)CCCCCCCC] | 8-(heptadecan-9-yloxy)- 8-oxooctyl (2S)-4-(3- (dimethylamino)propoxy)- 1-(6-oxo-6- (undecyloxy)hexyl) pyrrolidine-2-carboxylate |
| 6 | 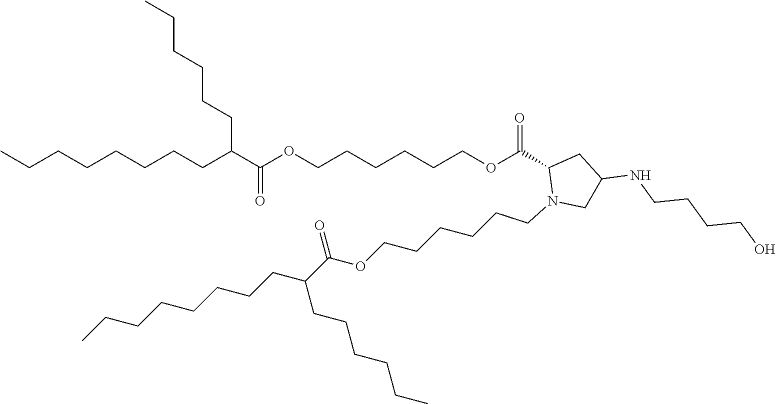 [SMILES: CCCCCCCCC(CCCCCC)C(=O)OCCCCCCOC(=O)[C@@H]1CC(NCCCCO)CN1CCCCCCOC(=O)C(CCCCCC)CCCCCCCC] | 6-((2- hexyldecanoyl)oxy)hexyl (2S)-1-(6-((2- hexyldecanoyl)oxy)hexyl)- 4-((4- hydroxybutyl)amino) pyrrolidine-2-carboxylate |
| 7 | 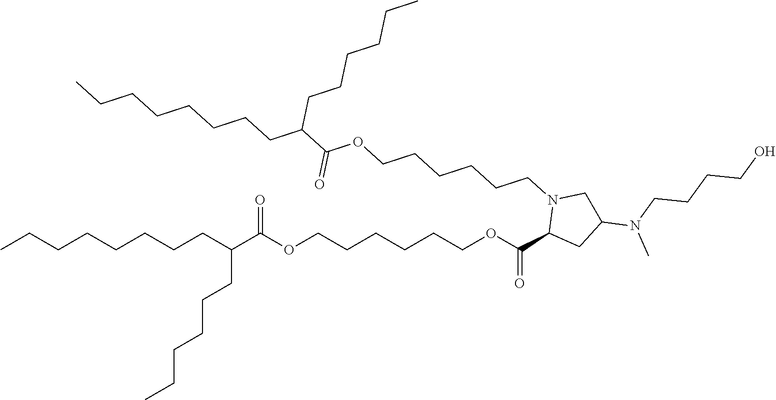 [SMILES: CCCCCCCCC(CCCCCC)C(=O)OCCCCCCOC(=O)[C@@H]1CC(N(C)CCCCO)CN1CCCCCCOC(=O)C(CCCCCC)CCCCCCCC] | 6-((2- hexyldecanoyl)oxy)hexyl (2S)-1-(6-((2- hexyldecanoyl)oxy)hexyl)- 4-((4- hydroxybutyl)(methyl) amino)pyrrolidine-2- carboxylate |
| 8 | 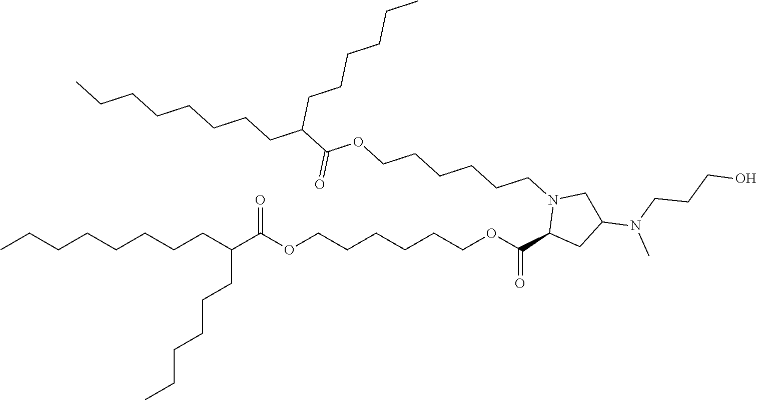 [SMILES: CCCCCCCCC(CCCCCC)C(=O)OCCCCCCOC(=O)[C@@H]1CC(N(C)CCCO)CN1CCCCCCOC(=O)C(CCCCCC)CCCCCCCC] | 6-((2- hexyldecanoyl)oxy)hexyl (2S)-1-(6-((2- hexyldecanoyl)oxy)hexyl)- 4-((3- hydroxypropyl)(methyl) amino)pyrrolidine-2- carboxylate |
| 9 | 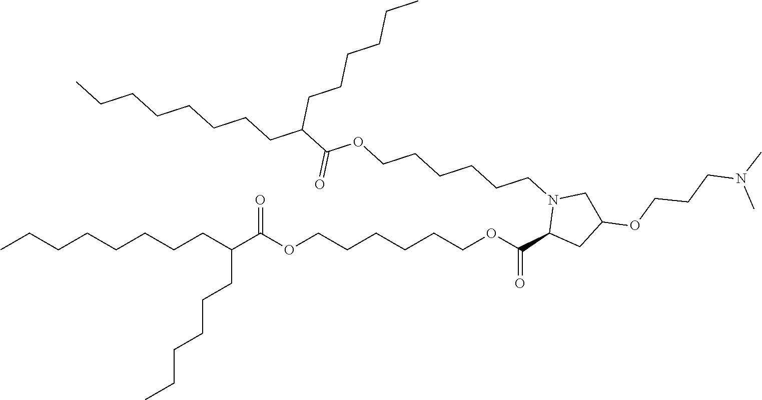 [SMILES: CCCCCCCCC(CCCCCC)C(=O)OCCCCCCOC(=O)[C@@H]1CC(OCCCN(C)C)CN1CCCCCCOC(=O)C(CCCCCC)CCCCCCCC] | 6-((2- hexyldecanoyl)oxy)hexyl (2S)-4-(3- (dimethylamino)propoxy)- 1-(6-((2- hexyldecanoyl)oxy)hexyl) pyrrolidine-2- carboxylate |
| 10 | 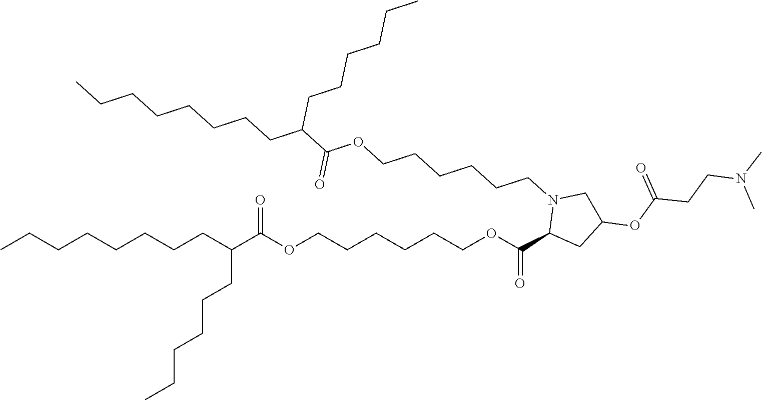 [SMILES: CCCCCCCCC(CCCCCC)C(=O)OCCCCCCOC(=O)[C@@H]1CC(OC(=O)CCN(C)C)CN1CCCCCCOC(=O)C(CCCCCC)CCCCCCCC] | 6-((2- hexyldecanoyl)oxy)hexyl (2S)-4-((3- (dimethylamino)propanoyl) oxy)-1-(6-((2- hexyldecanoyl)oxy)hexyl) pyrrolidine-2- carboxylate |
| 11 | 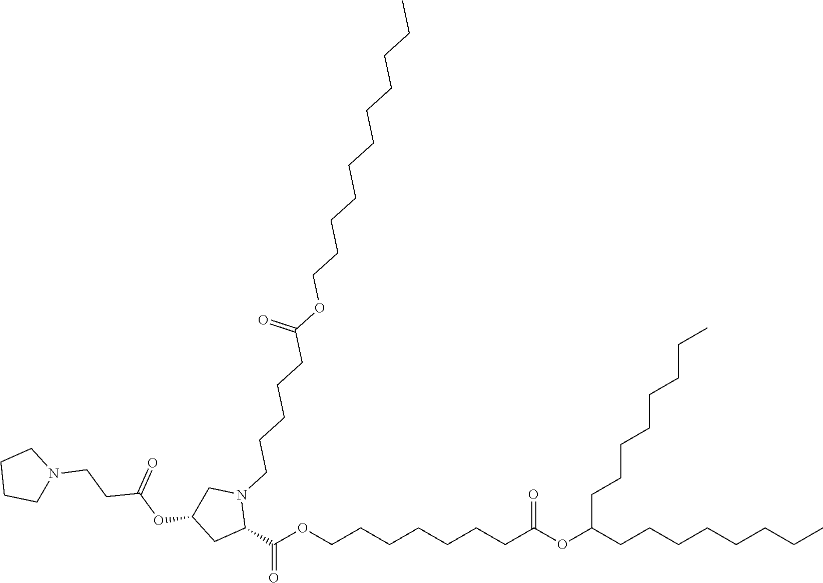 [SMILES: CCCCCCCCCCCOC(=O)CCCCCN1C[C@@H](OC(=O)CCN2CCCC2)C[C@H]1C(=O)OCCCCCCCC(=O)OC(CCCCCCCC)CCCCCCCC] | 8-(heptadecan-9-yloxy)-8- oxooctyl (2S,4S)-1-(6- oxo-6- (undecyloxy)hexyl)-4-((3- (pyrrolidin-1- yl)propanoyl)oxy) pyrrolidine-2-carboxylate |
| 12. | 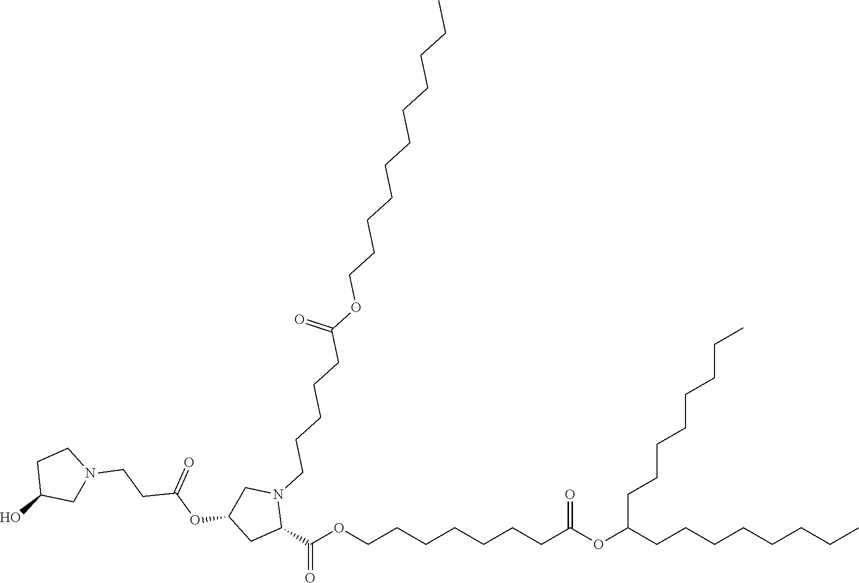 [SMILES: CCCCCCCCCCCOC(=O)CCCCCN1C[C@@H](OC(=O)CCN2CC[C@H](O)C2)C[C@H]1C(=O)OCCCCCCCC(=O)OC(CCCCCCCC)CCCCCCCC] | 8-(heptadecan-9-yloxy)-8- oxooctyl (2S,4S)-4-((3- ((S)-3-hydroxypyrrolidin- 1-yl)propanoyl)oxy)-1-(6- oxo-6- (undecyloxy)hexyl) pyrrolidine-2-carboxylate |
| 13. | 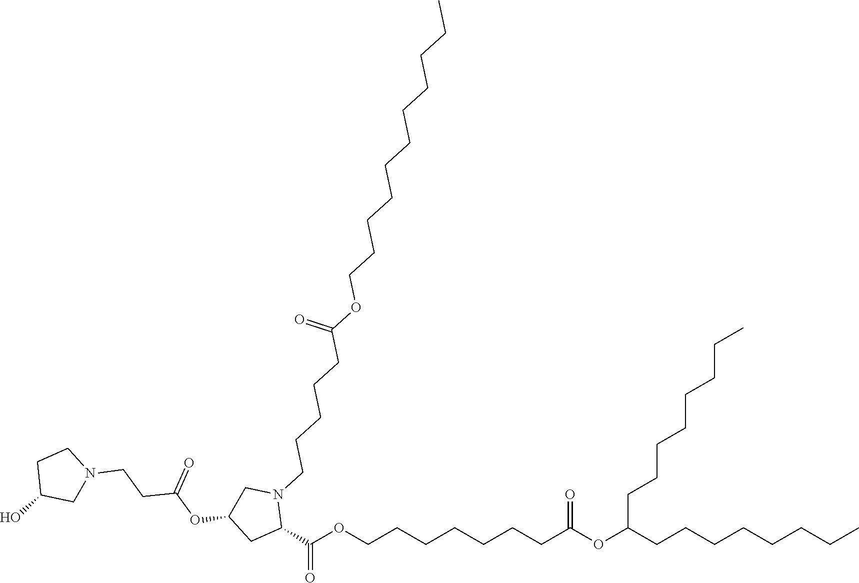 [SMILES: CCCCCCCCCCCOC(=O)CCCCCN1C[C@@H](OC(=O)CCN2CC[C@@H](O)C2)C[C@H]1C(=O)OCCCCCCCC(=O)OC(CCCCCCCC)CCCCCCCC] | 8-(heptadecan-9-yloxy)-8- oxooctyl (2S,4S)-4-((3- ((R)-3-hydroxypyrrolidin- 1-yl)propanoyl)oxy)-1-(6- oxo-6- (undecyloxy)hexyl) pyrrolidine-2-carboxylate |
| 14. | 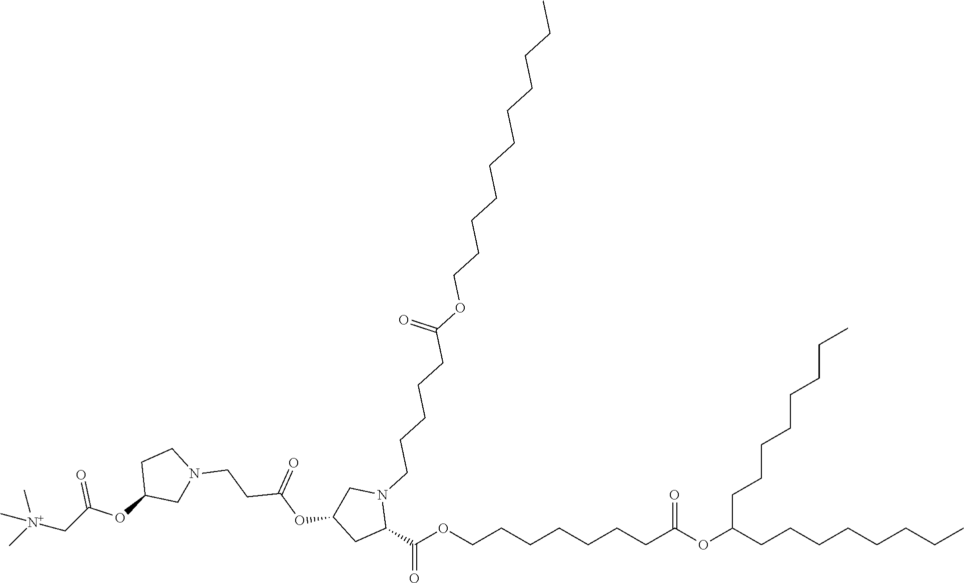 [SMILES: CCCCCCCCCCCOC(=O)CCCCCN1C[C@@H](OC(=O)CCN2CC[C@H](OC(=O)C[N+](C)(C)C)C2)C[C@H]1C(=O)OCCCCCCCC(=O)OC(CCCCCCCC)CCCCCCCC] | 2-(((S)-1-(3-(((3S,5S)-5- (((8-(heptadecan-9-yloxy)- 8-oxooctyl)oxy)carbonyl)- 1-(6-oxo-6- (undecyloxy)hexyl) pyrrolidin-3-yl)oxy)-3- oxopropyl)pyrrolidin-3- yl)oxy)-N,N,N-trimethyl- 2-oxoethan-1-aminium |
| 15. | 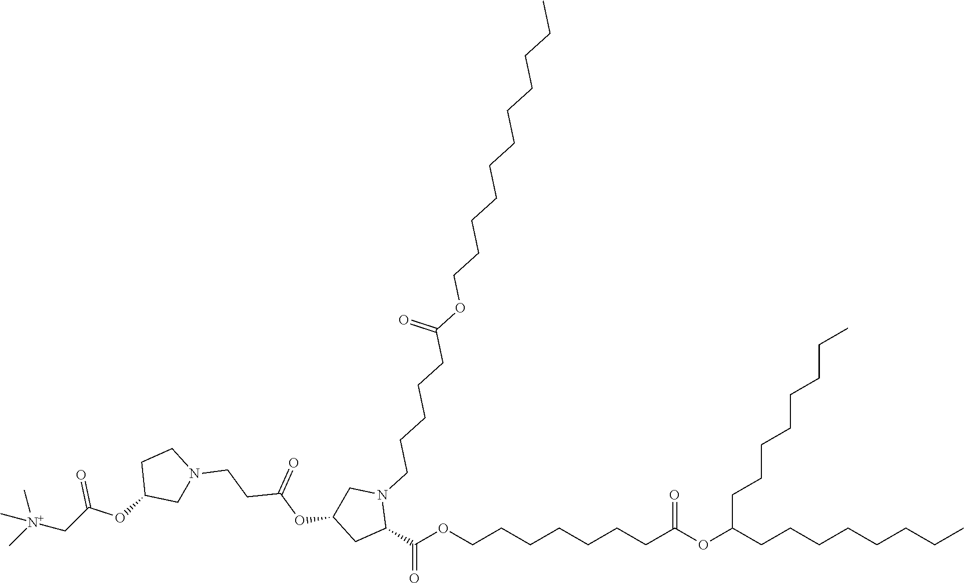 [SMILES: CCCCCCCCCCCOC(=O)CCCCCN1C[C@@H](OC(=O)CCN2CC[C@@H](OC(=O)C[N+](C)(C)C)C2)C[C@H]1C(=O)OCCCCCCCC(=O)OC(CCCCCCCC)CCCCCCCC] | 2-(((R)-1-(3-(((3S,5S)-5- (((8-(heptadecan-9-yloxy)- 8-oxooctyl)oxy)carbonyl)- 1-(6-oxo-6- (undecyloxy)hexyl) pyrrolidin-3-yl)oxy)-3- oxopropyl)pyrrolidin-3- yl)oxy)-N,N,N-trimethyl- 2-oxoethan-1-aminium |
| 16. | 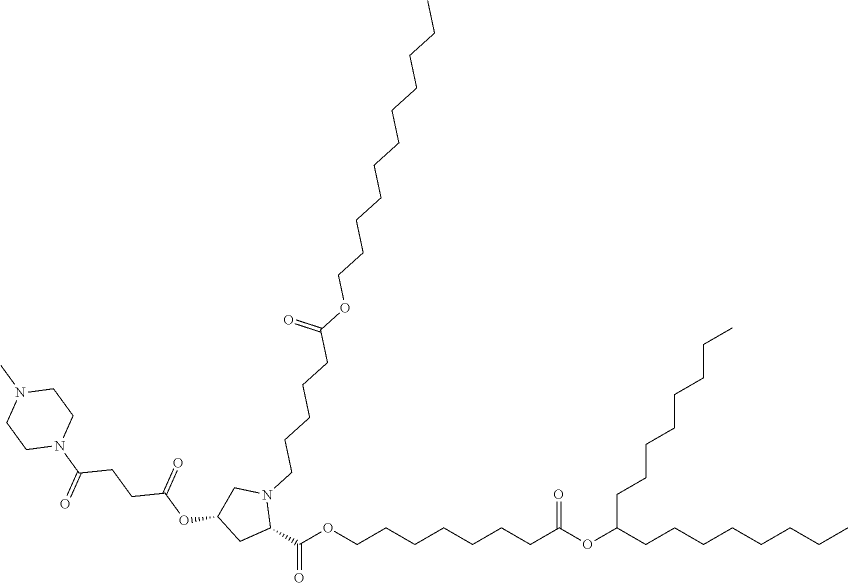 [SMILES: CCCCCCCCCCCOC(=O)CCCCCN1C[C@@H](OC(=O)CCC(=O)N2CCN(C)CC2)C[C@H]1C(=O)OCCCCCCCC(=O)OC(CCCCCCCC)CCCCCCCC] | 8-(heptadecan-9-yloxy)-8- oxooctyl (2S,4S)-4-((4-(4- methylpiperazin-1-yl)-4- oxobutanoyl)oxy)-1-(6- oxo-6- (undecyloxy)hexyl) pyrrolidine-2-carboxylate |
| 17. | 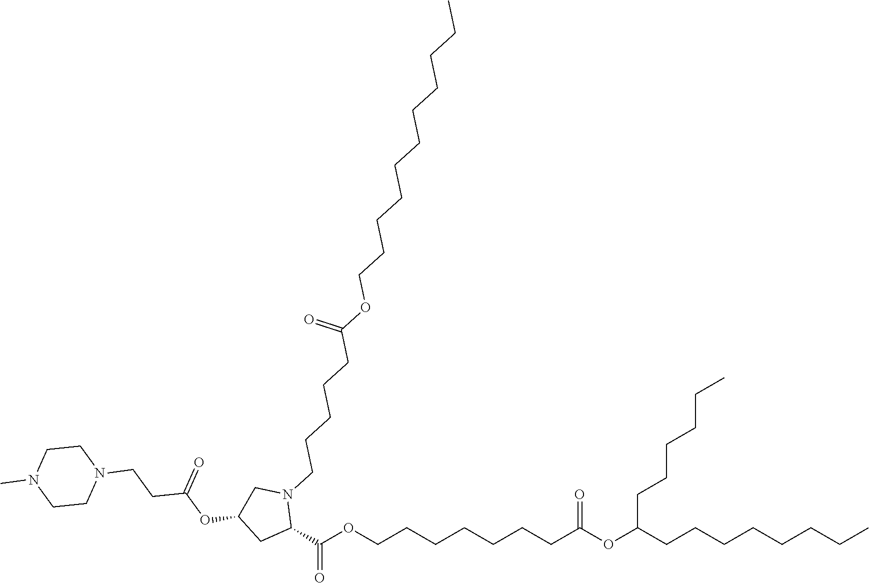 [SMILES: CCCCCCCCCCCOC(=O)CCCCCN1C[C@@H](OC(=O)CCN2CCN(C)CC2)C[C@H]1C(=O)OCCCCCCCC(=O)OC(CCCCCC)CCCCCCCC] | 8-oxo-8-(pentadecan-7- yloxy)octyl (2S,4S)-4-((3- (4-methylpiperazin-1- yl)propanoyl)oxy)-1-(6- oxo-6- (undecyloxy)hexyl) pyrrolidine-2-carboxylate |
| 18. | 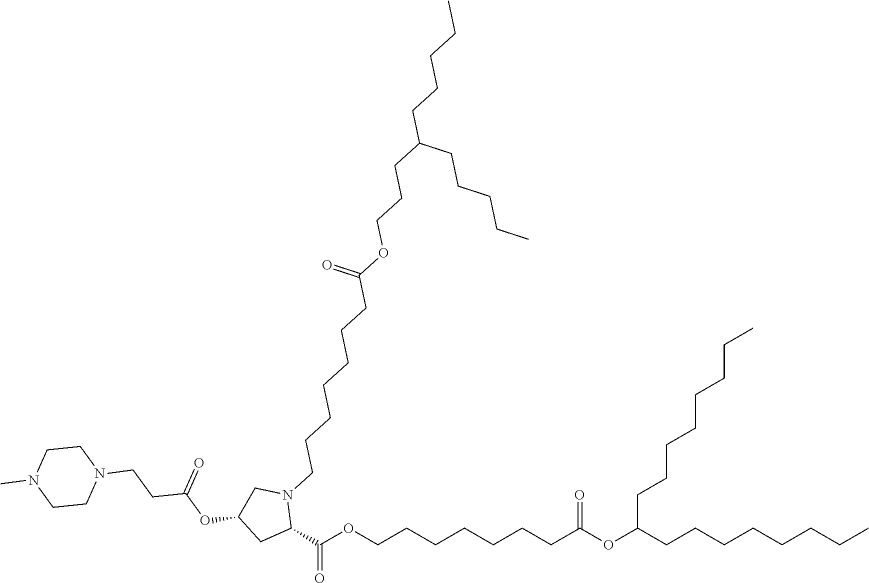 [SMILES: CCCCCCCCC(CCCCCCCC)OC(=O)CCCCCCCOC(=O)[C@@H]1C[C@H](OC(=O)CCN2CCN(C)CC2)CN1CCCCCCCC(=O)OCCCC(CCCCC)CCCCC] | 8-(heptadecan-9-yloxy)-8- oxooctyl (2S,4S)-4-((3-(4- methylpiperazin-1- yl)propanoyl)oxy)-1-(8- oxo-8-((4- pentylnonyl)oxy)octyl) pyrrolidine-2-carboxylate |
| 19. | 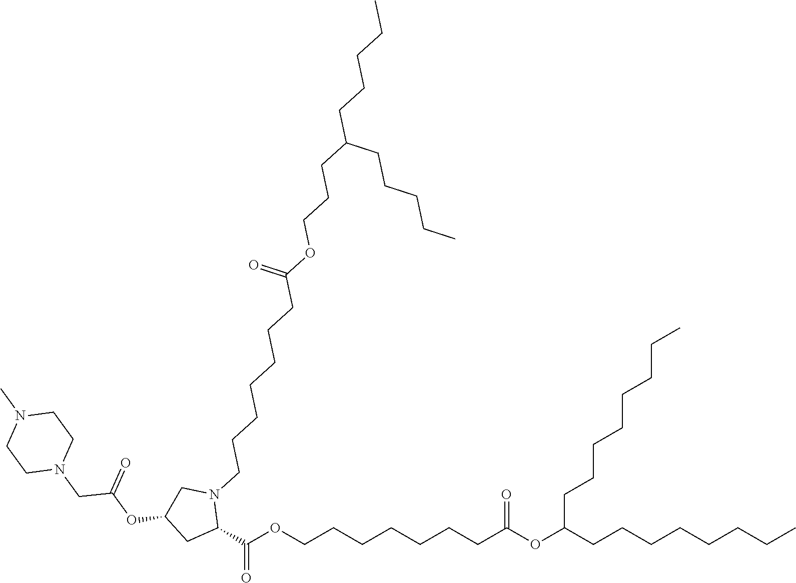 [SMILES: CCCCCCCCC(CCCCCCCC)OC(=O)CCCCCCCOC(=O)[C@@H]1C[C@H](OC(=O)CN2CCN(C)CC2)CN1CCCCCCCC(=O)OCCCC(CCCCC)CCCCC] | 8-(heptadecan-9-yloxy)-8- oxooctyl (2S,4S)-4-(2-(4- methylpiperazin-1- yl)acetoxy)-1-(8-oxo-8- ((4- pentylnonyl)oxy)octyl) pyrrolidine-2-carboxylate |
| 20. | 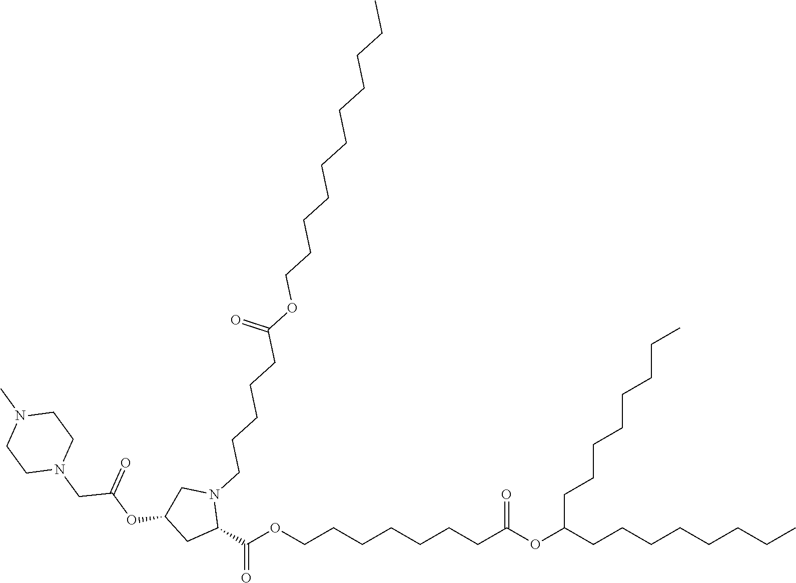 [SMILES: CCCCCCCCCCCOC(=O)CCCCCN1C[C@@H](OC(=O)CN2CCN(C)CC2)C[C@H]1C(=O)OCCCCCCCC(=O)OC(CCCCCCCC)CCCCCCCC] | 8-(heptadecan-9-yloxy)-8- oxooctyl (2S,4S)-4-(2-(4- methylpiperazin-1- yl)acetoxy)-1-(6-oxo-6- (undecyloxy)hexyl) pyrrolidine-2-carboxylate |
| 21. | 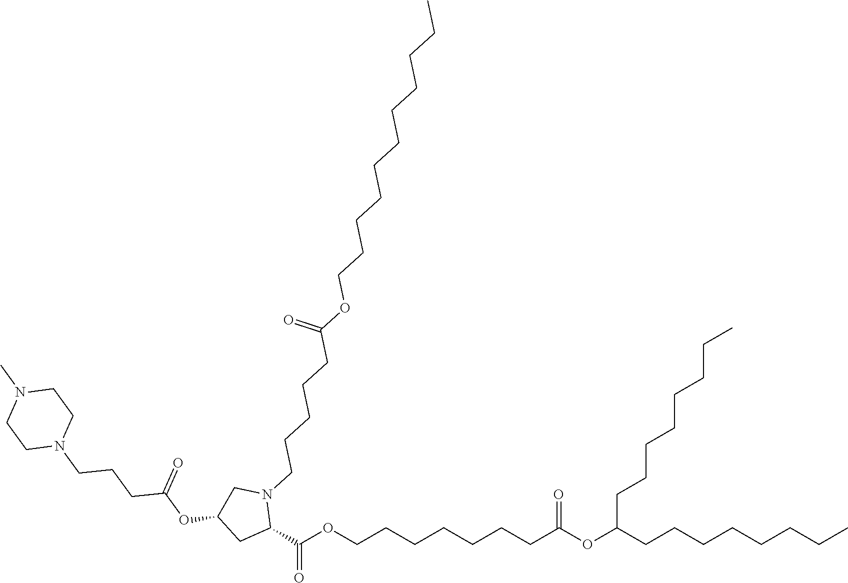 [SMILES: CCCCCCCCCCCOC(=O)CCCCCN1C[C@@H](OC(=O)CCCN2CCN(C)CC2)C[C@H]1C(=O)OCCCCCCCC(=O)OC(CCCCCCCC)CCCCCCCC] | 8-(heptadecan-9-yloxy)-8- oxooctyl (2S,4S)-4-((4-(4- methylpiperazin-1- yl)butanoyl)oxy)-1-(6- oxo-6- (undecyloxy)hexyl) pyrrolidine-2-carboxylate |
| 22. | 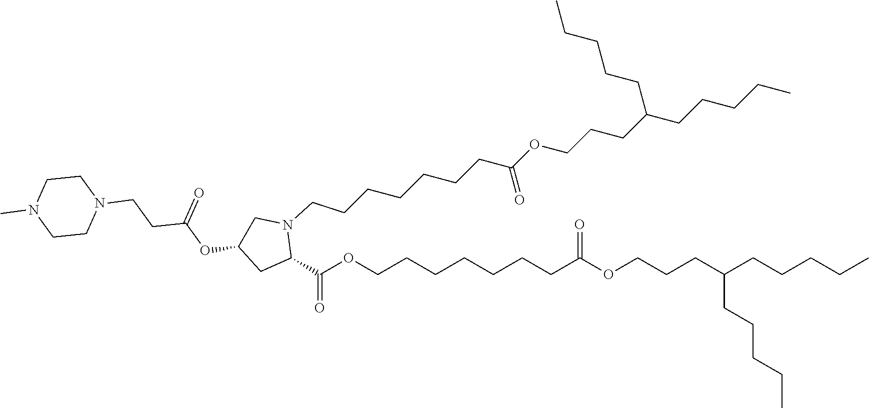 [SMILES: CCCCCC(CCCCC)CCCOC(=O)CCCCCCCOC(=O)[C@@H]1C[C@H](OC(=O)CCN2CCN(C)CC2)CN1CCCCCCCC(=O)OCCCC(CCCCC)CCCCC] | 8-oxo-8-((4- pentylnonyl)oxy)octyl (2S,4S)-4-((3-(4- methylpiperazin-1- yl)propanoyl)oxy)-1-(8- oxo-8-((4- pentylnonyl)oxy)octyl) pyrrolidine-2-carboxylate |
| 23. | 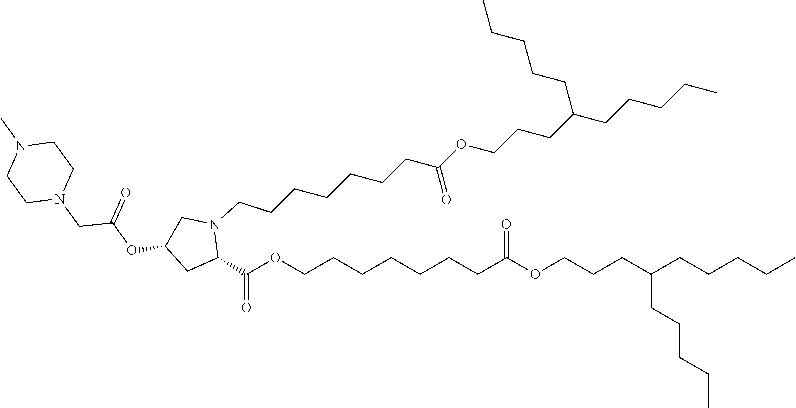 [SMILES: CCCCCC(CCCCC)CCCOC(=O)CCCCCCCOC(=O)[C@@H]1C[C@H](OC(=O)CN2CCN(C)CC2)CN1CCCCCCCC(=O)OCCCC(CCCCC)CCCCC] | 8-oxo-8-((4- pentylnonyl)oxy)octyl (2S,4S)-4-(2-(4- methylpiperazin-1- yl)acetoxy)-1-(8-oxo-8- ((4- pentylnonyl)oxy)octyl) pyrrolidine-2-carboxylate |
| 24. | 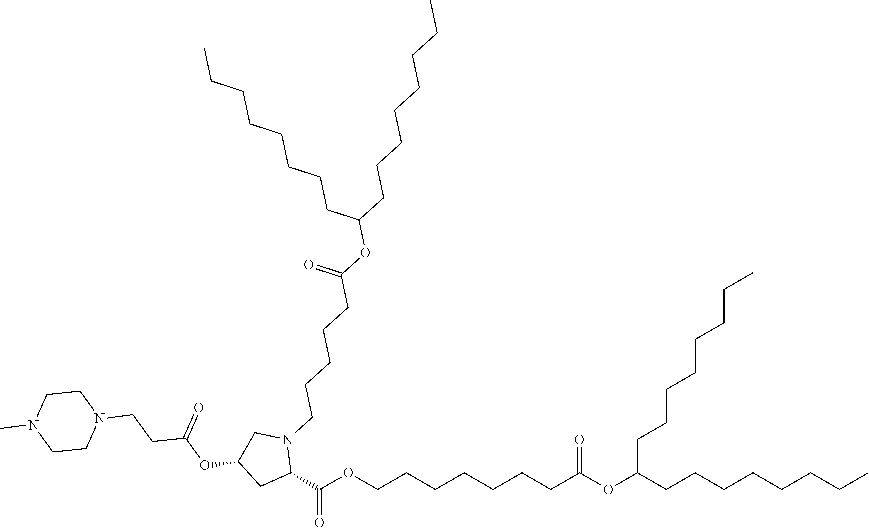 [SMILES: CCCCCCCCC(CCCCCCCC)OC(=O)CCCCCCCOC(=O)[C@@H]1C[C@H](OC(=O)CCN2CCN(C)CC2)CN1CCCCCC(=O)OC(CCCCCCCC)CCCCCCCC] | 8-(heptadecan-9-yloxy)-8- oxooctyl (2S,4S)-1-(6- (heptadecan-9-yloxy)-6- oxohexyl)-4-((3-(4- methylpiperazin-1- yl)propanoyl)oxy) pyrrolidine-2-carboxylate |
| 25. | 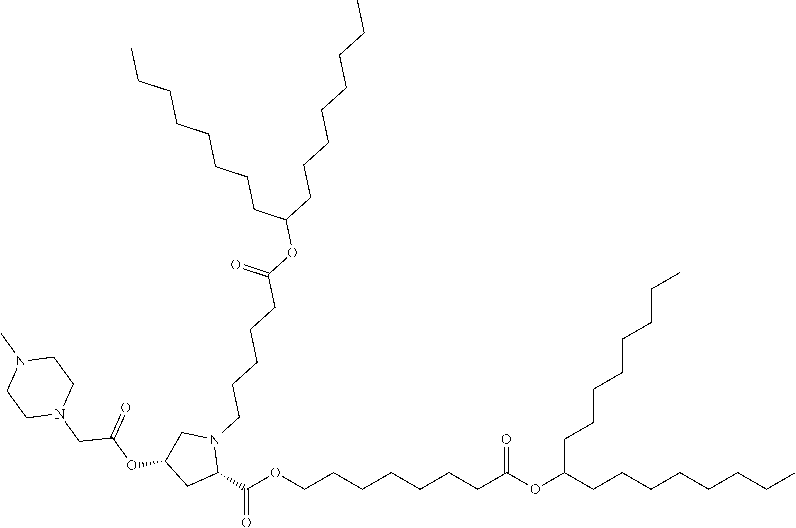 [SMILES: CCCCCCCCC(CCCCCCCC)OC(=O)CCCCCCCOC(=O)[C@@H]1C[C@H](OC(=O)CN2CCN(C)CC2)CN1CCCCCC(=O)OC(CCCCCCCC)CCCCCCCC] | 8-(heptadecan-9-yloxy)-8- oxooctyl (2S,4S)-1-(6- (heptadecan-9-yloxy)-6- oxohexyl)-4-(2-(4- methylpiperazin-1- yl)acetoxy)pyrrolidine-2- carboxylate |
| 26. | 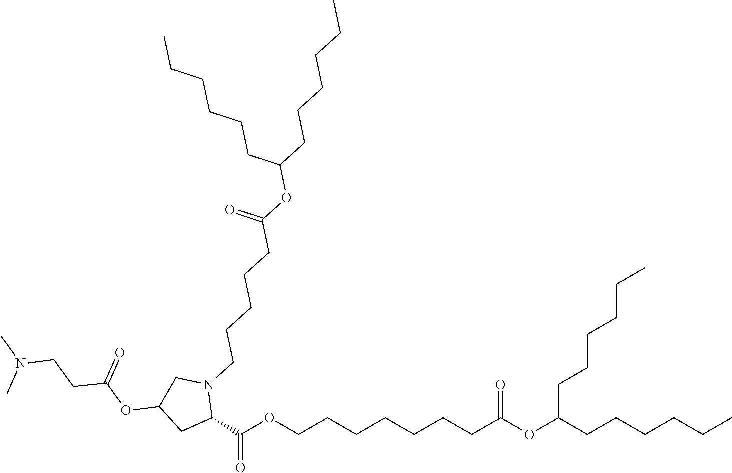 [SMILES: CCCCCCC(CCCCCC)OC(=O)CCCCCCCOC(=O)[C@@H]1CC(OC(=O)CCN(C)C)CN1CCCCCC(=O)OC(CCCCCC)CCCCCC] | 8-oxo-8-(tridecan-7- yloxy)octyl (2S)-4-((3- (dimethylamino)propanoyl) oxy)-1-(6-oxo-6- (tridecan-7- yloxy)hexyl)pyrrolidine-2- carboxylate |
| 27. | 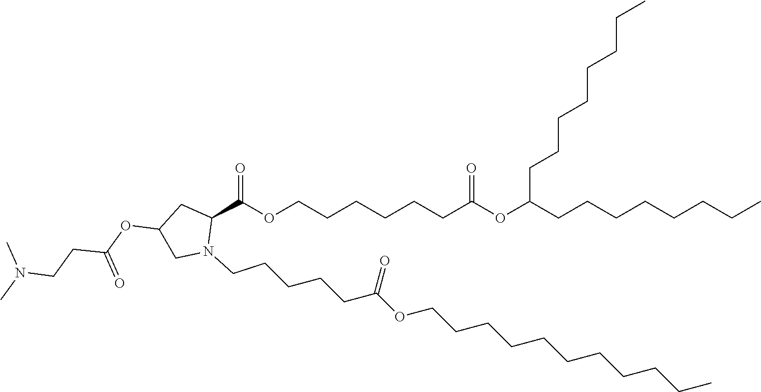 [SMILES: CCCCCCCCCCCOC(=O)CCCCCN1CC(OC(=O)CCN(C)C)C[C@H]1C(=O)OCCCCCCC(=O)OC(CCCCCCCC)CCCCCCCC] | 7-(heptadecan-9-yloxy)-7- oxoheptyl (2S)-4-((3- (dimethylamino)propanoyl) oxy)-1-(6-oxo-6- (undecyloxy)hexyl) pyrrolidine-2-carboxylate |
| 28. | 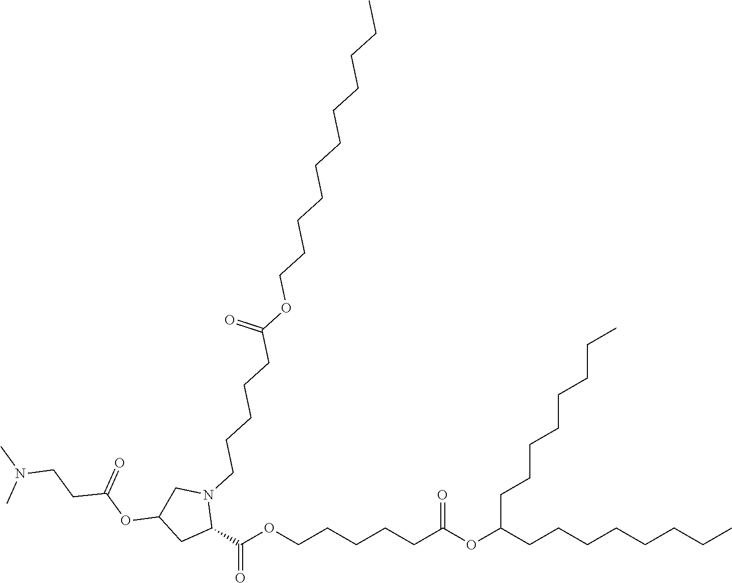 [SMILES: CCCCCCCCCCCOC(=O)CCCCCN1CC(OC(=O)CCN(C)C)C[C@H]1C(=O)OCCCCCC(=O)OC(CCCCCCCC)CCCCCCCC] | 6-(heptadecan-9-yloxy)-6- oxohexyl (2S)-4-((3- (dimethylamino)propanoyl) oxy)-1-(6-oxo-6- (undecyloxy)hexyl) pyrrolidine-2-carboxylate |
| 29. | 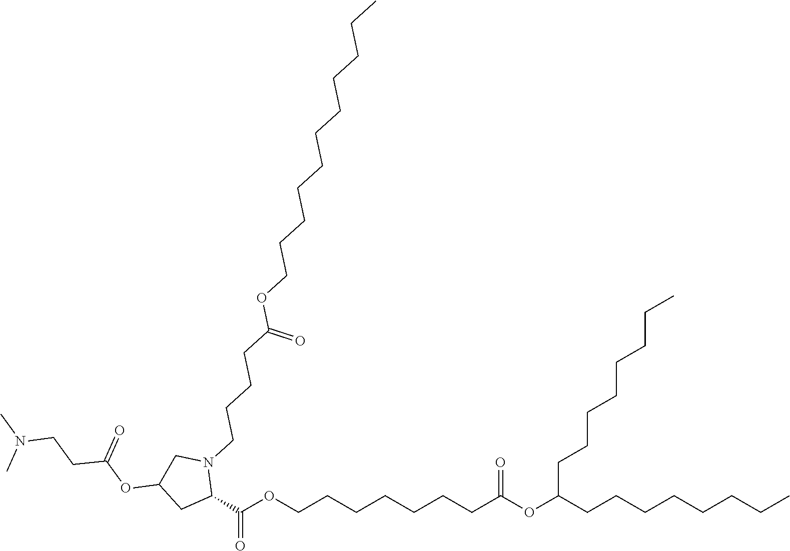 [SMILES: CCCCCCCCCCCOC(=O)CCCCN1CC(OC(=O)CCN(C)C)C[C@H]1C(=O)OCCCCCCCC(=O)OC(CCCCCCCC)CCCCCCCC] | 8-(heptadecan-9-yloxy)-8- oxooctyl (2S)-4-((3- (dimethylamino)propanoyl) oxy)-1-(5-oxo-5- (undecyloxy)pentyl) pyrrolidine-2-carboxylate |
| 30. | 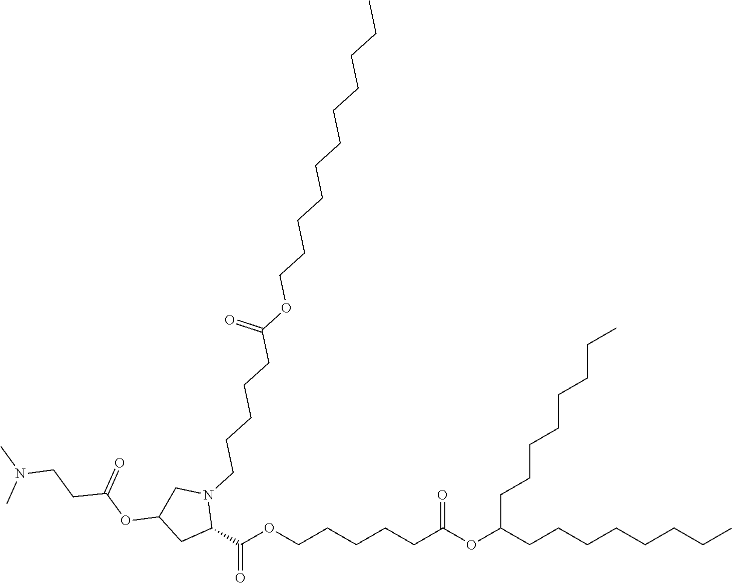 [SMILES: CCCCCCCCCCCOC(=O)CCCCCN1CC(OC(=O)CCN(C)C)C[C@H]1C(=O)OCCCCCC(=O)OC(CCCCCCCC)CCCCCCCC] | 6-(heptadecan-9-yloxy)-6- oxohexyl (2S)-4-((3- (dimethylamino)propanoyl) oxy)-1-(6-oxo-6- (undecyloxy)hexyl) pyrrolidine-2-carboxylate |
| 31. | 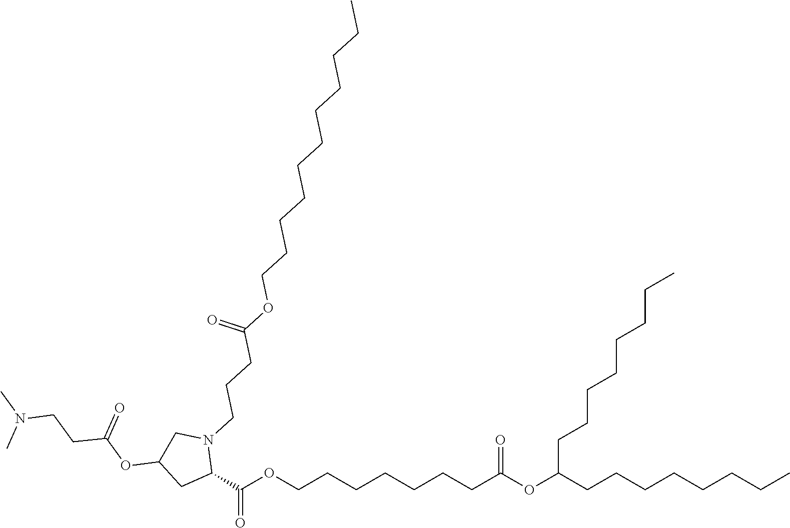 [SMILES: CCCCCCCCCCCOC(=O)CCCN1CC(OC(=O)CCN(C)C)C[C@H]1C(=O)OCCCCCCCC(=O)OC(CCCCCCCC)CCCCCCCC] | 8-(heptadecan-9-yloxy)-8- oxooctyl (2S)-4-((3- (dimethylamino)propanoyl) oxy)-1-(4-oxo-4- (undecyloxy)butyl) pyrrolidine-2-carboxylate |
| 32. | 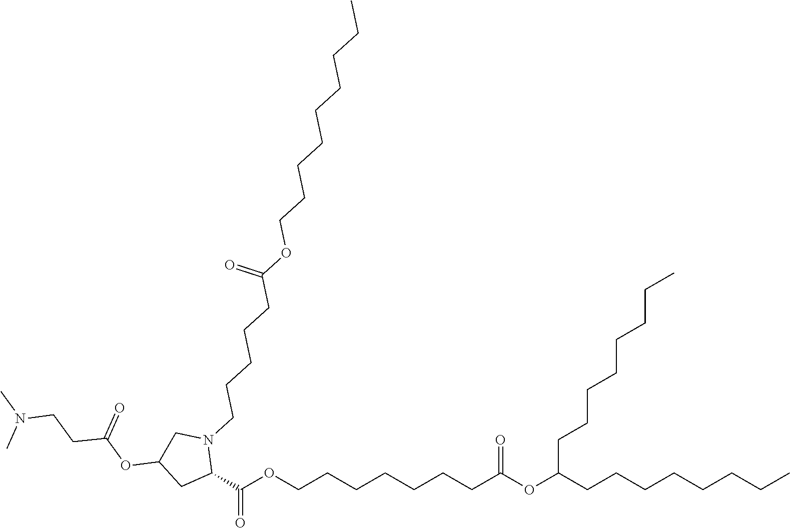 [SMILES: CCCCCCCCCOC(=O)CCCCCN1CC(OC(=O)CCN(C)C)C[C@H]1C(=O)OCCCCCCCC(=O)OC(CCCCCCCC)CCCCCCCC] | 8-(heptadecan-9-yloxy)-8- oxooctyl (2S)-4-((3- (dimethylamino)propanoyl) oxy)-1-(6-(nonyloxy)-6- oxohexyl)pyrrolidine-2- carboxylate |
| 33. | 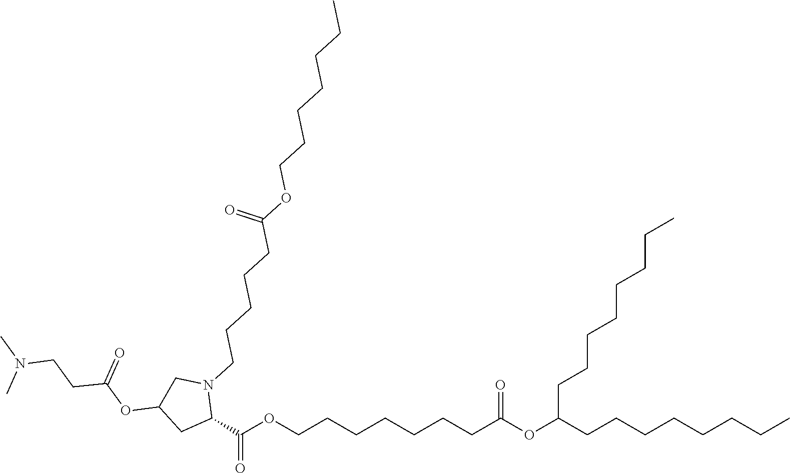 [SMILES: CCCCCCCCC(CCCCCCCC)OC(=O)CCCCCCCOC(=O)[C@@H]1CC(OC(=O)CCN(C)C)CN1CCCCCC(=O)OCCCCCCC] | 8-(heptadecan-9-yloxy)-8- oxooctyl (2S)-4-((3- (dimethylamino)propanoyl) oxy)-1-(6-(heptyloxy)-6- oxohexyl)pyrrolidine-2- carboxylate |

### Lipid Composition

The ionizable lipids disclosed herein may be used to form lipid compositions. Thus, another aspect of the invention relates to a lipid composition comprising a lipid compound as described herein in the above of the invention relating to the novel ionizable lipid compounds.

All above descriptions and all embodiments discussed in the above aspects relating to the lipid compounds, including the compounds covered by formula (I), (IA-1), (IA-2), (IIA)-(IIC), (IIA-1), (IIIA)-(IIIIE), (IIIC-1), (IVA-1)-(IVA-3), (IVC-1)-(IVC-2); and (VC-1)-(VC-6) are all applicable to these aspects of the invention relating to the lipid compositions.

As described herein, suitable lipid compounds to be used in the lipid composition include all the isomers and isotopes of the compounds described above, as well as all the pharmaceutically acceptable salts, solvates, or hydrates thereof, and all crystal forms, crystal form mixtures, and anhydrides or hydrates.

In some embodiments, the lipid composition contains one or more compounds described herein, or a pharmaceutically acceptable salt thereof. In some embodiments, the lipid composition is a liposome or a lipid nanoparticle (LNP). In one embodiment, the lipid composition is a LNP.

In addition to one or more compounds described herein, the lipid composition may further include a second lipid. In some embodiments, the disclosure relates to a lipid composition comprising (i) one or more lipid compounds chosen from the ionizable lipids of Formula (I), (IA-1), (IA-2), (IIA)-(IIC), (IIA-1), (IIIA)-(IIIIE), (IIIC-1), (IVA-1)-(IVA-3), (IVC-1)-(IVC-2), and (VC-1)-(VC-6), pharmaceutically acceptable salts thereof, and stereoisomers of any of the foregoing and (ii) a second lipid. In some embodiments, the lipid composition comprises 5%, 10%, 15%, 20%, 25%, 30%, 35%, 40%, 45%, 50%, 55%, 60%, 65%, 70%, 75%, 80%, 85%, 90%, or 95% of the one or more lipid compounds.

In some embodiments, the second lipid is cationic, non-cationic (e.g., neutral, anionic, or zwitterionic), or ionizable. In some embodiments, the lipid composition comprises about a 1:1 ratio of the lipid compound and the second lipid (e.g., a helper lipid).

In some embodiments, the second lipid is a cationic lipid, anionic lipid, another ionizable lipid, or zwitterionic lipid.

In some embodiments, the disclosure relates to a lipid natoparticle composition comprising (i) one or more ionizable lipid compounds as described herein and (ii) one or more lipid components.

In some embodiments, the one or more lipid components in the lipid composition comprise one or more helper lipids and one or more PEG lipids. In some embodiments, the lipid component(s) comprise(s) one or more helper lipids, one or more PEG lipids, and one or more neutral lipids. In some embodiments, the lipid composition may further comprise a sterol and a PEG lipid. In some embodiments, the lipid composition may further comprise a sterol, a PEGylated lipid, a phospholipid, and/or a neutral lipid.

In some embodiments, one or more naturally occurring and/or synthetic lipid compounds may be used in the preparation of the lipid composition. The lipid composition may contain negatively charged lipids, positively charged lipids, or a combination thereof.

### The Non-Ionizable Lipid Components

### Charged and Neutral Lipids

Examples of suitable negatively charged (anionic) lipids include, but are not limited to dimyrystoyl-, dipalmitoyl-, and distearoyl-phasphatidylglycerol; dimyrystoyl-, dipalmitoyl-, and dipalmitoyl-phosphatidic acid; dimyrystoyl-, dipalmitoyl-, and dipalmitoyl-phosphatidylethanolamine; and their unsaturated diacyl and mixed acyl chain counterparts as well as cardiolipin.

Examples of positively charged (cationic) lipids include, but are not limited to, N,N′-dimethyl-N,N′-dioctacyl ammonium bromide (DDAB) and chloride DDAC), N-(1-(2,3-dioleyloxy)propyl)-N,N,N-trimethylammonium chloride (DOTMA), 3β-[N—(N′,N′-dimethylaminoethyl)carbamoyl) cholesterol (DC-chol), 1,2-dioleoyloxy-3-[trimethylammonio]-propane (DOTAP), 1,2-dioctadecyloxy-3-[trimethylammonio]-propane (DSTAP), and 1,2-dioleoyloxypropyl-3-dimethyl-hydroxy ethyl ammonium chloride (DORI), and the cationic lipids described in e.g. Martin et al., Current Pharmaceutical Design, pages 1-394, which is herein incorporated by reference in its entirety.

Additional exemplary cationic lipids include, but are not limited to, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-Dioleoyl-3-Dimethylammonium-propane (DODAP), 1,2-Dioleoylcarbamyl-3-Dimethylammonium-propane (DOCDAP), 1,2-Dilineoyl-3-Dimethylammonium-propane (DLINDAP), 3-Dimethylamino-2-(Cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis, cis-9′,12′-octadecadienoxy)propane (CpLin DMA), N,N-Dimethyl-3,4-dioleyloxybenzylamine (DMOBA) and/or a mixture thereof. The neutral lipid can comprise dioleoylphosphatidylethanolamine (DOPE), palmitoyloleoylphosphatidylcholine (POPC), egg phosphatidylcholine (EPC), distearoylphosphatidylcholine (DSPC), and/or a mixture thereof.

In some embodiments, the lipid components comprise one or more neutral lipids. The neutral lipids may be one or more phospholipids, such as one or more (poly)unsaturated lipids. Phospholipids may assemble into one or more lipid bilayers. In general, phospholipids may include a phospholipid moiety and one or more fatty acid moieties. For example, a phospholipid may be a lipid according to formula:

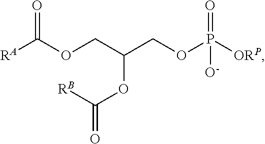

wherein R^{p} represents a phospholipid moiety, and R^{A} and R^{B} represent fatty acid moieties with or without unsaturation that may be the same or different. A phospholipid moiety may be a phosphatidyl choline, phosphatidyl ethanolamine, phosphatidyl glycerol, phosphatidyl serine, phosphatidic acid, 2-lysophosphatidyl choline, or a sphingomyelin. A fatty acid moiety may be a lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, or docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a lipid nanoparticle to facilitate membrane permeation or cellular recognition or in conjugating a lipid nanoparticle to a useful component such as a targeting or imaging moiety (e.g., a dye).

In some embodiments, the neutral lipids may be phospholipids such as distearoyl-sn-glycero-3-phosphocholine (DSPC), 1,2-dioleoyl-sn-glycero-3-phosphoethanolamine (DOPE), 1,2-dilinolcoyl-sn-glycero-3-phosphocholine (DLPC), 1,2-dimyristoyl-sn-glycero-phosphocholine (DMPC), 1,2-dioleoyl-sn-glycero-3-phospioclioline (DOPC), 1,2-dipalmitoyl-sn-glycero-3-phosphocholine (DPPC), 1,2-diundecanoyl-sn-glycero-3-phosphocholine (DUPC), 1-palmitoyl-2-oleoyl-sn-glycero-3-phosphocholine (POPC), 1,2-di-O-octadecenyl-sn-glycero-3-phosphocholine (18:0 dieher PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMP), 1-stearoyl-2-oleoyl-phosphatidyethanolanine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), or mixtures thereof.

Additional non-limiting examples of neutral lipids also include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids may be acyl groups derived from fatty acids having C₁₀-C₂₄ carbon chains, e.g., lauroyl, myristoyl, palmitoyl, stearoyl, or oleoyl.

### Steroids and Other Non-Ionizable Lipid Components

In some embodiments, the lipid components in the lipid composition comprise one or more steroids or analogues thereof.

In some embodiments, the lipid components in the lipid composition comprise sterols such as cholesterol, sisterol and derivatives thereof. Non-limiting examples of cholesterol derivatives include polar analogues such as 5a-cholestanol, 5a-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5a-cholestanone, and cholesteryl decanoate; and mixtures thereof. In some embodiments, the cholesterol derivative is a polar analogue such as cholesteryl-(4′-hydroxy)-butyl ether.

In some embodiments, the non-ionizable lipid components comprises or consists of a mixture of one or more phospholipids and cholesterol or a derivative thereof. In some embodiments, the non-ionizable lipid components present in the lipid composition comprises or consists of one or more phospholipids, e.g., a cholesterol-free lipid particle formulation. In some embodiments, the non-ionizable lipid components present in the lipid composition comprises or consists of cholesterol or a derivative thereof, e.g., a phospholipid-free lipid particle formulation.

In some embodiments, the lipid components in the lipid composition (e.g., LNP composition) comprises a phytosterol or a combination of a phytosterol and cholesterol. In some embodiments, the phytosterol is selected from the group consisting of b-sitosterol, stigmasterol, b-sitostanol, campesterol, brassicasterol, and combinations thereof. In some embodiments, the phytosterol is selected from the group consisting of b-sitosterol, b-sitostanol, campesterol, brassicasterol, Compound S-140, Compound S-151, Compound S-156, Compound S-157, Compound S-159, Compound S-160, Compound S-164, Compound S-165, Compound S-170, Compound S-173, Compound S—175 and combinations thereof. In some embodiments, the phytosterol is selected from the group consisting of Compound S-140, Compound S-151, Compound S-156, Compound S-157, Compound S-159, Compound S-160, Compound S-164, Compound S-165, Compound S-170, Compound S-173, Compound S-175, and combinations thereof. In some embodiments, the phytosterol is a combination of Compound S-141, Compound S-140, Compound S-143 and Compound S-148. In some embodiments, the phytosterol comprises a sitosterol or a salt or an ester thereof.

In some embodiments, the phytosterol comprises a stigmasterol or a salt or an ester thereof.

In some embodiments, the phytosterol is beta-sitosterol,

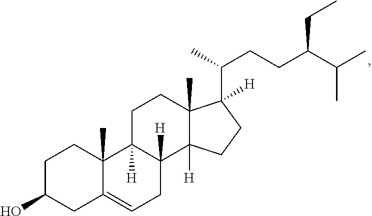

a salt thereof, or an ester thereof.

In some embodiments, the lipid composition (e.g., LNP composition) comprises a phytosterol, or a salt or ester thereof, and cholesterol or a salt thereof.

In some embodiments, the target delivery cell for the lipid composition is a cell described herein (e.g., a liver cell or a splenic cell), and the phytosterol or a salt or ester thereof is selected from the group consisting of b-sitosterol, b-sitostanol, campesterol, and brassicasterol, and combinations thereof. In some embodiments, the phytosterol is b-sitosterol. In some embodiments, the phytosterol is b-sitostanol. In some embodiments, the phytosterol is campesterol. In some embodiments, the phytosterol is brassicasterol.

In some embodiments, the target delivery cell for the lipid composition is a cell described herein (e.g., a liver cell or a splenic cell), and the phytosterol or a salt or ester thereof is selected from the group consisting of b-sitosterol, and stigmasterol, and combinations thereof. In some embodiments, the phytosterol is b-sitosterol. In some embodiments, the phytosterol is stigmasterol.

Other examples of non-ionizable lipid components include nonphosphorous containing lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

In some embodiments, the non-ionizable lipid components are present from 10 mol % to 60 mol %, from 20 mol % to 55 mol %, from 20 mol % to 45 mol %, 20 mol % to 40 mol %, from 25 mol % to 50 mol %, from 25 mol % to 45 mol %, from 30 mol % to 50 mol %, from 30 mol % to 45 mol %, from 30 mol % to 40 mol %, from 35 mol % to 45 mol %, from 37 mol % to 42 mol %, or 35 mol %, 36 mol %, 37 mol %, 38 mol %, 39 mol %, 40 mol %, 41 mol %, 42 mol %, 43 mol %, 44 mol %, or 45 mol % (or any fraction thereof or range therein) of the total lipid present in the lipid composition.

In the embodiments where the lipid compositions contain a mixture of phospholipid and cholesterol or a cholesterol derivative, the mixture may be present up to 40 mol %, 45 mol %, 50 mol %, 55 mol %, or 60 mol % of the total lipid present in the lipid composition.

In some embodiments, the phospholipid component in the mixture may be present from 2 mol % to 20 mol %, from 2 mol % to 15 mol %, from 2 mol % to 12 mol %, from 4 mol % to 15 mol %, or from 4 mol % to 10 mol % (or any fraction thereof or range therein) of the total lipid present in the lipid composition. In some embodiments, the phospholipid component in the mixture be present from 5 mol % to 10 mol %, from 5 mol % to 9 mol %, from 5 mol % to 8 mol %, from 6 mol % to 9 mol %, from 6 mol % to 8 mol %, or 5 mol %, 6 mol %, 7 mol %, 8 mol %, 9 mol %, or 10 mol % (or any fraction thereof or range therein) of the total lipid present in the lipid composition.

In some embodiments, the sterol component (e.g. cholesterol component) in the mixture may be present from 25 mol % to 45 mol %, from 25 mol % to 40 mol %, from 30 mol % to 45 mol %, from 30 mol % to 40 mol %, from 27 mol % to 37 mol %, from 25 mol % to 30 mol %, or from 35 mol % to 40 mol % (or any fraction thereof or range therein) of the total lipid present in the lipid composition. In some embodiments, the cholesterol component in the mixture be present from 25 mol % to 35 mol %, from 27 mol % to 35 mol %, from 29 mol % to 35 mol %, from 30 mol % to 35 mol %, from 30 mol % to 34 mol %, from 31 mol % to 33 mol %, or 30 mol %, 31 mol %, 32 mol %, 33 mol %, 34 mol %, or 35 mol % (or any fraction thereof or range therein) of the total lipid present in the lipid composition.

In embodiments where the lipid compositions are phospholipid-free, the cholesterol or derivative thereof may be present up to 25 mol %, 30 mol %, 35 mol %, 40 mol %, 45 mol %, 50 mol %, 55 mol %, or 60 mol % of the total lipid present in the lipid composition.

In some embodiments, the sterol component (e.g. cholesterol or derivative thereof) in the phospholipid-free lipid particle formulation may be present from 25 mol % to 45 mol %, from 25 mol % to 40 mol %, from 30 mol % to 45 mol %, from 30 mol % to 40 mol %, from 31 mol % to 39 mol %, from 32 mol % to 38 mol %, from 33 mol % to 37 mol %, from 35 mol % to 45 mol %, from 30 mol % to 35 mol %, from 35 mol % to 40 mol %, or 30 mol %, 31 mol %, 32 mol %, 33 mol %, 34 mol %, 35 mol %, 36 mol %, 37 mol %, 38 mol %, 39 mol %, or 40 mol % (or any fraction thereof or range therein) of the total lipids present in the lipid composition.

In some embodiments, the non-ionizable lipid components may be present from 5 mol % to 90 mol %, from 10 mol % to 85 mol %, from 20 mol % to 80 mol %, 10 mol % (e.g., phospholipid only), or 60 mol % (e.g., phospholipid and cholesterol or derivative thereof) (or any fraction thereof or range therein) of the total lipid present in the lipid composition.

The percentage of non-ionizable lipid present in the lipid composition is a target amount, and that the actual amount of non-ionizable lipid present in the particle may vary, for example, by 5 mol %.

### Lipid Conjugates

The lipid composition described herein may further comprise one or more lipid conjugates. A conjugated lipid may prevent the aggregation of particles. Non-limiting examples of conjugated lipids include PEG-lipid conjugates, cationic polymer-lipid conjugates, and mixtures thereof.

In some embodiments, the lipid conjugate is a PEG-lipid or PEG-modified lipid (alternatively referred to as PEGylated lipid). A PEG lipid is a lipid modified with polyethylene glycol. Examples of PEG-lipids include, but are not limited to, PEG coupled to dialkyloxypropyls (PEG-DAA), PEG coupled to diacylglycerol (PEG-DAG), PEG-modified dialkylamines, PEG-modified diacylglycerols (PEG-DEG), PEG coupled to phospholipids such as phosphatidylethanolamine (PEG-PE), PEG-modified phosphatidic acids, PEG conjugated to ceramides (PEG-CER), PEG conjugated to cholesterol or a derivative thereof, and mixtures thereof.

For example, a PEG lipid may be PEG-c-DOMG, PEG-DMG, PEG-DLPE, PEG-DMPE, PEG-DPPC, or a PEG-DSPE lipid.

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol.

In some embodiments, the PEG-lipid is selected from the group consisting of 1,2-dimyristoyl-sn-glycerol methoxypolyethylene glycol (PEG-DMG), 1,2-distearoyl-sn-glycero-3-phosphoethanolamine-N-[amino(polyethylene glycol)](PEC-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

PEG is a linear, water-soluble polymer of ethylene PEG repeating units with two terminal hydroxyl groups. PEGs are classified by their molecular weights; and include the following: monomethoxypoly ethylene glycol (MePEG-OH), monomethoxypoly ethylene glycol-succinate (MePEG-S), monomethoxypoly ethylene glycol-succinimidyl succinate (MePEG-S—NHS), monomethoxypoly ethylene glycol-amine (MePEG-NH₂), monomethoxypoly ethylene glycol-tresylate (MePEG-TRES), monomethoxypoly ethylene glycol-imidazolyl-carbonyl (MePEG-IM), as well as such compounds containing a terminal hydroxyl group instead of a terminal methoxy group (e.g., HO-PEG-S, HO-PEG-S—NHS, HO-PEG-NH₂).

The PEG moiety of the PEG-lipid conjugates described herein may comprise an average molecular weight ranging from 550 daltons to 10,000 daltons. In certain instances, the PEG moiety has an average molecular weight of from 750 daltons to 5,000 daltons (e.g., from 1,000 daltons to 5,000 daltons, from 1,500 daltons to 3,000 daltons, from 750 daltons to 3,000 daltons, from 750 daltons to 2,000 daltons). In some embodiments, the PEG moiety has an average molecular weight of 2,000 daltons or 750 daltons.

In certain instances, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester-containing linker moieties and ester-containing linker moieties. In some embodiments, the linker moiety is a non-ester-containing linker moiety. Suitable non-ester-containing linker moieties include, but are not limited to, amido (—C(O)NH—), amino (—NR—), carbonyl (—C(O)—), carbamate (—NHC(O)O—), urea (—NHC(O)NH—), disulphide (—S—S—), ether (—O—), succinyl (—(O)CCH₂CH₂C(O)—), succinamidyl (—NHC(O)CH₂CH₂C(O)NH—), ether, disulphide, as well as combinations thereof (such as a linker containing both a carbamate linker moiety and an amido linker moiety). In some embodiments, a carbamate linker is used to couple the PEG to the lipid.

In some embodiments, an ester-containing linker moiety is used to couple the PEG to the lipid. Suitable ester-containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form the lipid conjugate. Such phosphatidylethanolamines are commercially available, or can be isolated or synthesized using conventional techniques known to those of skill in the art.

In some embodiments, phosphatidylethanolamines contain saturated or unsaturated fatty acids with carbon chain lengths in the range of C10 to C20. Phosphatidylethanolamines with mono- or di-unsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoyl-phosphatidylethanolamine (DOPE), and distearoyl-phosphatidylethanolamine (DSPE).

The term “diacylglycerol” or “DAG” includes a compound having 2 fatty acyl chains, R1 and R2, both of which have independently between 2 and 30 carbons bonded to the 1- and 2-position of glycerol by ester linkages. The acyl groups can be saturated or have varying degrees of unsaturation. Suitable acyl groups include, but are not limited to, lauroyl (C12), myristoyl (CM), palmitoyl (C16), stearoyl (C18), and icosoyl (C20). In some embodiments, R1 and R2 are the same, i.e., R1 and R2 are both myristoyl (i.e., dimyristoyl), R1 and R2 are both stearoyl (i.e., distearoyl).

The term “dialkyloxy propyl” or “DAA” includes a compound having 2 alkyl chains, R and R′, both of which have independently between 2 and 30 carbons. The alkyl groups can be saturated or have varying degrees of unsaturation.

In some embodiments, the PEG-DAA conjugate is a PEG-didecyloxypropyl (C10) conjugate, a PEG-dilauryloxypropyl (C12) conjugate, a PEG-dimyristyloxypropyl (C14) conjugate, a PEG-dipalmityloxy propyl (C16) conjugate, or a PEG-distearyloxy propyl (C18) conjugate. In some embodiments, the PEG has an average molecular weight of 750 or 2,000 daltons. In some embodiments, the terminal hydroxyl group of the PEG is substituted with a methyl group.

In addition to the foregoing, other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, poly gly colic acid, and derivatized celluloses such as hydroxymethylcellulose or hydroxy ethylcellulose.

In some embodiments, the PEG-lipid is a compound of formula

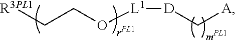

or a salt thereof, wherein:
R^{3PL1} is —OR^{OPL1};
R^{OPL1} is hydrogen, optionally substituted alkyl, or an oxygen protecting group;
r^{PL1} is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{NPL1}) S, C(O), C(O)N(R^{NPL1}), NR^{NPL1}C(O), —C(O)O, OC(O), OC(O)O, OC(O)N(R^{NPL1}), NR^{NPL1}C(O)O, or NR^{NPL1}C(O)N(R^{NPL1});
D is a moiety obtained by click chemistry or a moiety cleavable under physiological conditions; m^{PL1} is 0, 1, 2, 3, 4, 5, 6, 7, 8, 9, or 10;
A is of the formula:

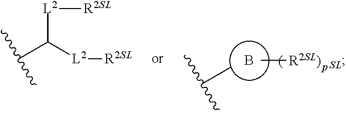

each instance of L² is independently a bond or optionally substituted C₁₋₆ alkylene, wherein one methylene unit of the optionally substituted C₁₋₆ alkylene is optionally replaced with O, N(R^{NPL1}), S, C(O), C(O)N(R^{NPL1}), NR^{NPL1}C(O), C(O)O, OC(O), OC(O)O, —OC(O)N(R^{NPL1}), NR^{NPL1}C(O)O, or NR^{NPL1}C(O)N(R^{NPL1});
each instance of R^{2SL} is independently optionally substituted C₁₋₃₀ alkyl, optionally substituted C₁₋₃₀ alkenyl, or optionally substituted C₁₋₃₀ alkynyl; optionally wherein one or more methylene units of R^{2SL} are independently replaced with optionally substituted carbocyclylene, optionally substituted heterocyclylene, optionally substituted arylene, optionally substituted heteroarylene, N(R^{NPL1}), O, S, C(O), C(O)N(R^{NPL1}), NR^{NPL1}C(O), —NR^{NPL1}C(O)N(R^{NPL1}), C(O)O, OC(O), OC(O)O, OC(O)N(R^{NPL1}), NR^{NPL1}C(O)O, C(O)S, —SC(O), C(═NR^{NPL1}), C(═NR^{NPL1})N(R^{NPL1}), NR^{NPL1}C(═NR^{NPL1}), —NR^{NPL1}C(═NR^{NPL1})N(R^{NPL1}), C(S), C(S)N(R^{NPL1}), NR^{NPL1}C(S), NR^{NPL1}C(S)N(R^{NPL1}), S(O), OS(O), S(O)O, OS(O)O, OS(O)₂, S(O)₂O, OS(O)₂O, N(R^{NPL1})S(O), S(O)N(R^{NPL1}), —N(R^{NPL1})S(O)N(R^{NPL1}), OS(O)N(R^{NPL1}), N(R^{NPL1})S(O)O, S(O)₂, N(R^{NPL1})S(O)₂, —S(O)₂N(R^{NPL1}), N(R^{NPL1})S(O)₂N(R^{NPL1}), OS(O)₂N(R^{NPL1}), or N(R^{NPL1})S(O)₂O;
each instance of R^{NPL1} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p^{SL} is 1 or 2.

In some embodiments, the PEG-lipid is a compound of formula

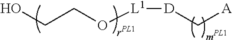

or a salt thereof, wherein r^{PL1}, L¹, D, m^{PL1}, and A are as above defined.

In some embodiments, the PEG-lipid is a compound of formula

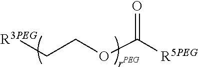

or a salt or isomer thereof, wherein:
R^{3PEG} is —OR^{O};
R^{O} is hydrogen, C₁₋₆ alkyl or an oxygen protecting group;
r^{PEG} is an integer between 1 and 100 (e.g., between 40 and 50, e.g., 45);
R^{5PEG} is C₁₀₋₄₀ alkyl (e.g., C₁₇ alkyl), C₁₀₋₄₀ alkenyl, or C₁₀₋₄₀, alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{NPEG})—, —O—, —S—, —C(O)—, —C(O)N(R^{NPEG})—, —NR^{NPEG}C(O)—, —NR^{NPEG}C(O)N(R^{NPEG})—, —C(O)O—, —OC(O)—, —OC(O)O—, —OC(O)N(R^{NPEG})—, —NR^{NPEG}C(O)O—, —C(O)S—, —SC(O)—, —C(═NR^{NPEG}C)—, —C(═NR^{NPEG})N(R^{NPEG})—, —NR^{NPEG}C(═NR^{NPEG})—, —NR^{NPEG}C(═NR^{NPEG})N(R^{NPEG})—, —C(S)—, —C(S)N(R^{NPEG})—, —NR^{NPEG}C(S)—, —NR^{NPEG}C(S)N(R^{NPEG})—, —S(O)—, —OS(O)—, —S(O)O—, —OS(O)O—, —OS(O)₂—, —S(O)₂O—, —OS(O)₂O—, —N(R^{NPEG})S(O)—, —S(O)N(R^{NPEG})—, —N(R^{NPEG})S(O)N(R^{NPEG}—, —OS(O)N(R^{NPEG})—, —N(R^{NPEG})S(O)O—, —S(O)₂—, —N(R^{NPEG})S(O)₂—, —S(O)₂N(R^{NPEG})—, —N(R^{NPEG})S(O)₂N(R^{NPEG})—, —OS(O)₂N(R^{NPEG})—, or —N(R^{NPEG})S(O)₂O—; and
each instance of R^{NPEG} is independently hydrogen, C₁₋₆ alkyl, or a nitrogen protecting group.

In some embodiments, the PEG-lipid is a compound of formula

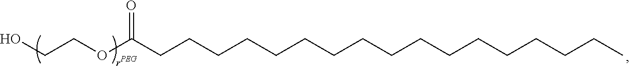

wherein r^{PEG} is an integer between 1 and 100 (e.g., between 40 and 50, e.g., 45).

In some embodiments, the PEG-lipid is a compound of formula

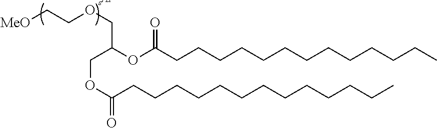

or a salt or isomer thereof, wherein s^{PL1} is an integer between 1 and 100 (e.g., between 40 and 50, e.g., 45).

In some embodiments, the PEG-lipid has the formula of

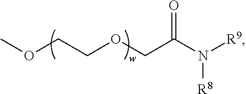

or a pharmaceutically acceptable salt, tautomer or stereoisomer thereof, wherein:
R⁸ and R⁹ are each independently a straight or branched, saturated or unsaturated alkyl chain containing form 10 to 30 carbon atoms, wherein the alkyl chain is optionally interrupted by one or more ester bonds (e.g., R⁸ and R⁹ are each independently straight, saturated alkyl chains containing form 12 to 16 carbon atoms); and
w has a mean value ranging from 30 to 60 (e.g., the average w is about 49).

In some embodiments, the incorporation of any of the above-discussed PEG-lipids in the lipid composition can improve the pharmacokinetics and/or biodistribution of the lipid composition. For example, incorporation of any of the above-discussed PEG-lipids in the lipid composition can reduce the accelerated blood clearance (ABC) effect.

### Other Ionizable Lipids

In some embodiments, the lipid composition may comprise one or more additional ionizable lipids, different than the ionizable lipids described herein. Exemplary ionizable lipids include, but are not limited to,

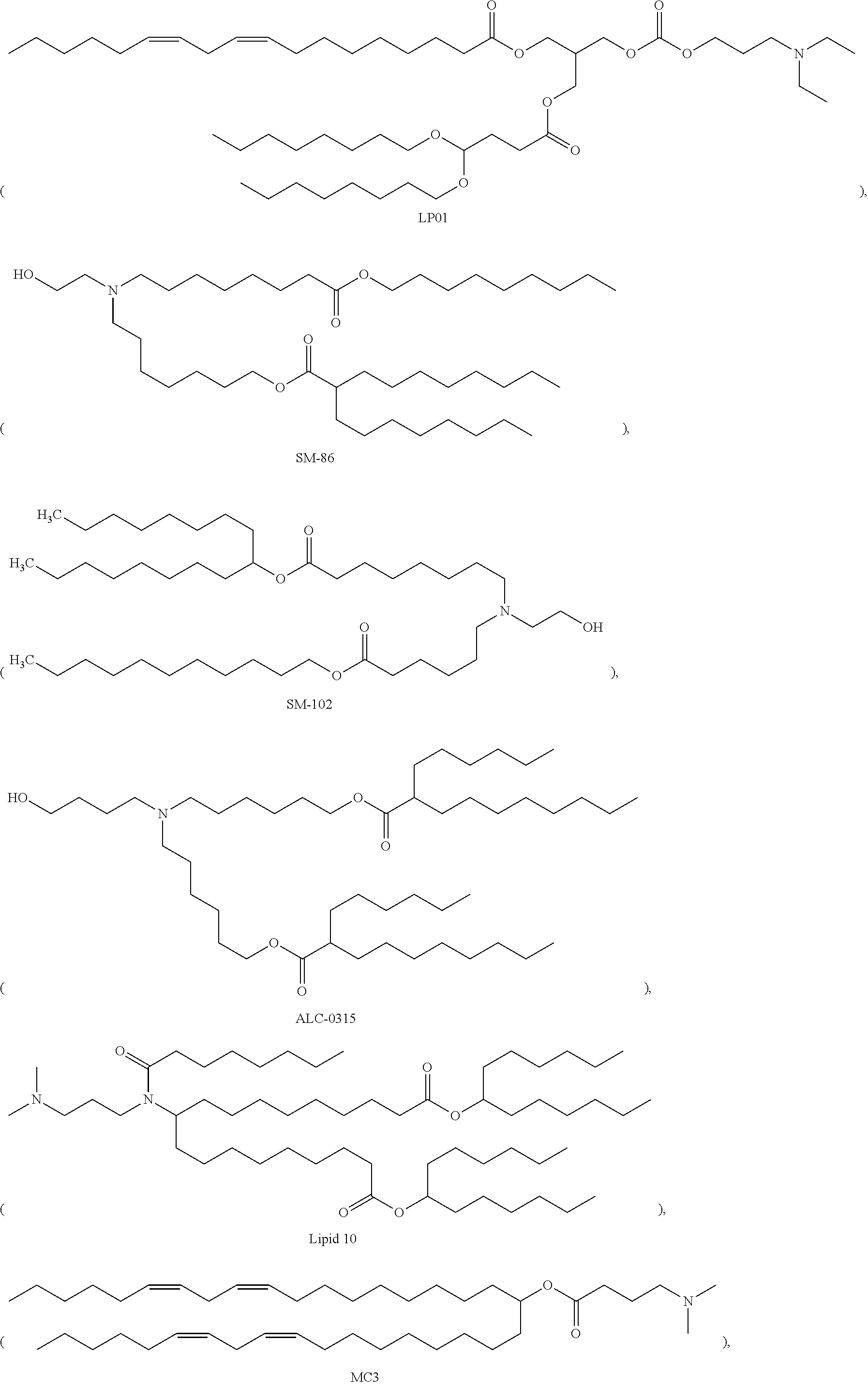

Acuitas Lipid 9, and Acuitas Lipid 10 (see WO 2017/004113A1 which is incorporated herein by reference in its entirety).

In one embodiment, the additional ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (which is incorporated by reference herein in its entirety).

In one embodiment, the additional ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01), e.g., as synthesized in Example 13 of WO 2015/095340 (which is incorporated by reference herein in its entirety).

In one embodiment, the additional ionizable lipid is Di((Z)-non-2-en-1-yl) 9-((4-dimethylamino)butanoyl)oxy)heptadecanedioate (L319), e.g., as synthesized in Example 7, 8, or 9 of US 2012/0027803 (which is incorporated by reference herein in its entirety).

In one embodiment, the additional ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO 2010/053572 (which is incorporated by reference herein in its entirety).

In one embodiment, the additional ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, e.g., Structure (I) from WO 2020/106946 (which is incorporated by reference herein in its entirety).

In one embodiment, the additional ionizable lipid is MC3 (6Z,9Z,28Z,31Z)-heptatriaconta-6,9,28,3 1-tetraen-19-yl-4-(dimethylamino) butanoate (DLin-MC3-DMA or MC3), e.g., as described in Example 9 of WO 2019/051289A9, which is incorporated by reference herein in its entirety.

In one embodiment, the additional ionizable lipid is lipid ATX-002, e.g., as described in Example 10 of WO 2019/051289A9, which incorporated by reference herein in its entirety.

In one embodiment, the additional ionizable lipid is (13Z,16Z)-A,A-dimethyl-3-nonyldocosa-13, 16-dien-1-amine (Compound 32), e.g., as described in Example 11 of WO 2019/051289A9 (which is incorporated by reference herein in its entirety).

In one embodiment, the additional ionizable lipid is Compound 6 or Compound 22, e.g., as described in Example 12 of WO 2019/051289A9, which is incorporated by reference herein in its entirety.

Examples of additional ionizable lipids useful in the lipid composition include those listed in Table 1 of WO 2019/051289, which is incorporated herein by reference.

### Additional Lipid Components

Some non-limiting examples of additional lipid compounds that may be used (e.g., in combination with the ionizable lipid compound described herein and other lipid components) to form the lipid composition include:

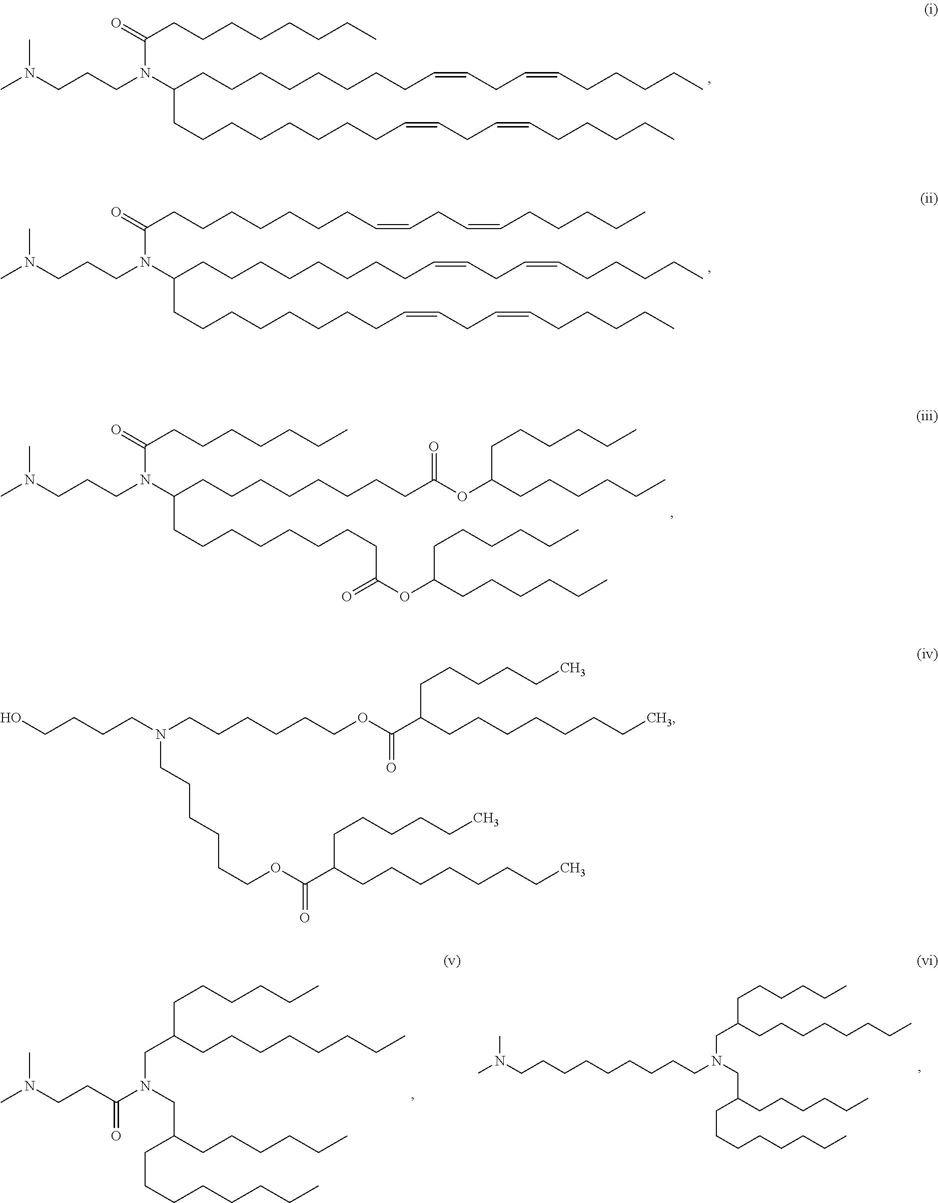

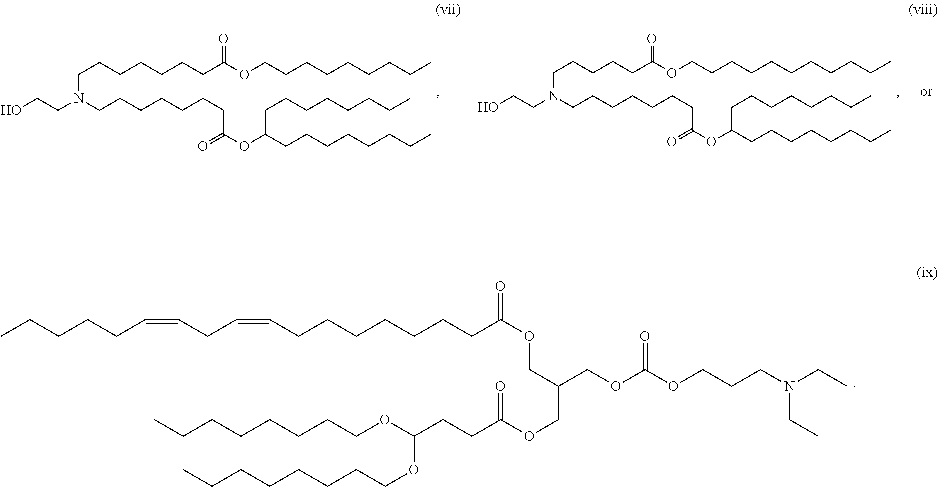 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCC/C=C\C/C=C\CCCCCCCC(=O)OCC(COC(=O)CCC(OCCCCCCCC)OCCCCCCCC)COC(=O)OCCCN(CC)CC]

In some embodiments, the lipid composition further comprises the lipids in formula (i), (ii), (iii), (iv), (v), (vi), (vii), (viii), or (ix).

In some embodiments, the lipid composition further comprises the following compounds having the structure of:

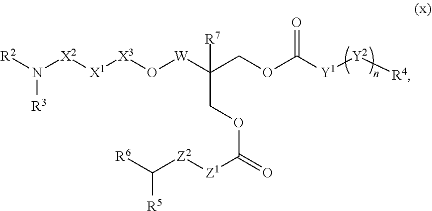

wherein:
X¹ is O, NR¹, or a direct bond, X² is C₂₋₅ alkylene, and X³ is C(═O) or a direct bond;
R¹ is H or Me, R³ is C₁₋₃ alkyl, R² is C₁₋₃ alkyl, or R² taken together with the nitrogen atom to which it is attached and 1-3 carbon atoms of X² form a 4-, 5-, or 6-membered ring; or
X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring;
Y¹ is C₂₋₁₂ alkylene, and Y² is selected from

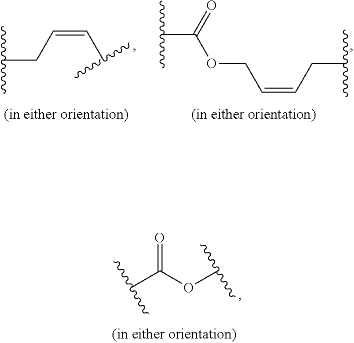 [SMILES: */C=C\C* ; *C/C=C\COC(*)=O ; *OC(*)=O]

n is 0 to 3;
R⁴ is C₁₋₁₅ alkyl;
Z¹ is C₁₋₆ alkylene or a direct bond, and Z² is

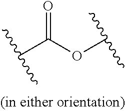 [SMILES: *OC(*)=O]

or absent, provided that if Z¹ is a direct bond, Z² is absent;
R⁵ is C₅₋₉ alkyl or C₆₋₁₀ alkoxy, R⁶ is C₅₋₉ alkyl or C₆₋₁₀ alkoxy;
W is methylene or a direct bond; and
R⁷ is H or Me, or a salt thereof;
provided that if R³ and R² are C₂ alkyls, X¹ is O, X² is linear C₃ alkylene, X³ is C(═O), Y¹ is linear C₅ alkylene, (Y²)n-R⁴ is:

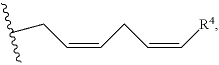

R⁴ is linear C₅ alkyl, Z¹ is C₂ alkylene, Z² is absent, W is methylene, and R⁷ is H, then R⁵ and R⁶ are not C₂ alkoxy. In some embodiments, the lipid composition further comprises one or more compounds of formula (x).

Additional non-limiting examples of lipid compounds that may be further included in the lipid composition further comprises (e.g., in combination with the lipid compounds described herein and other lipid components):

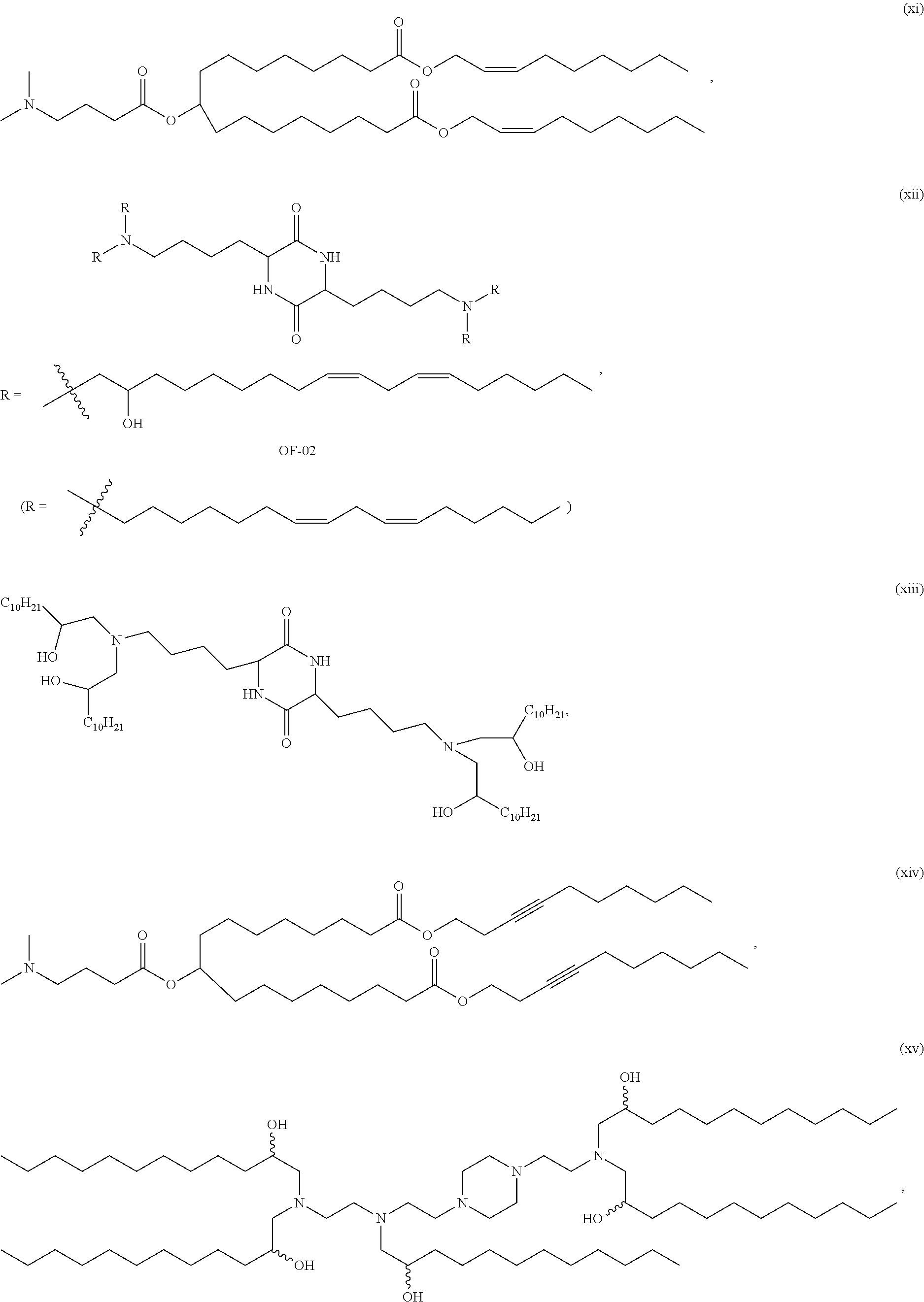

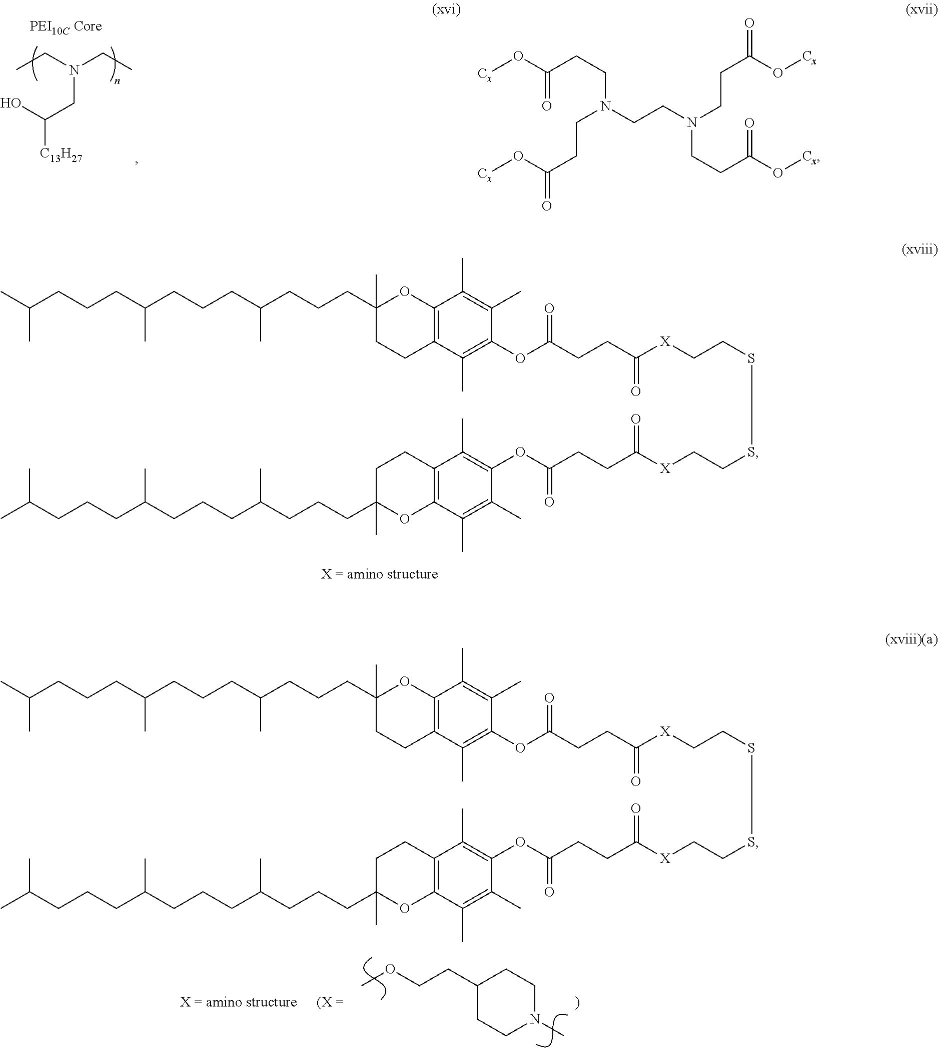

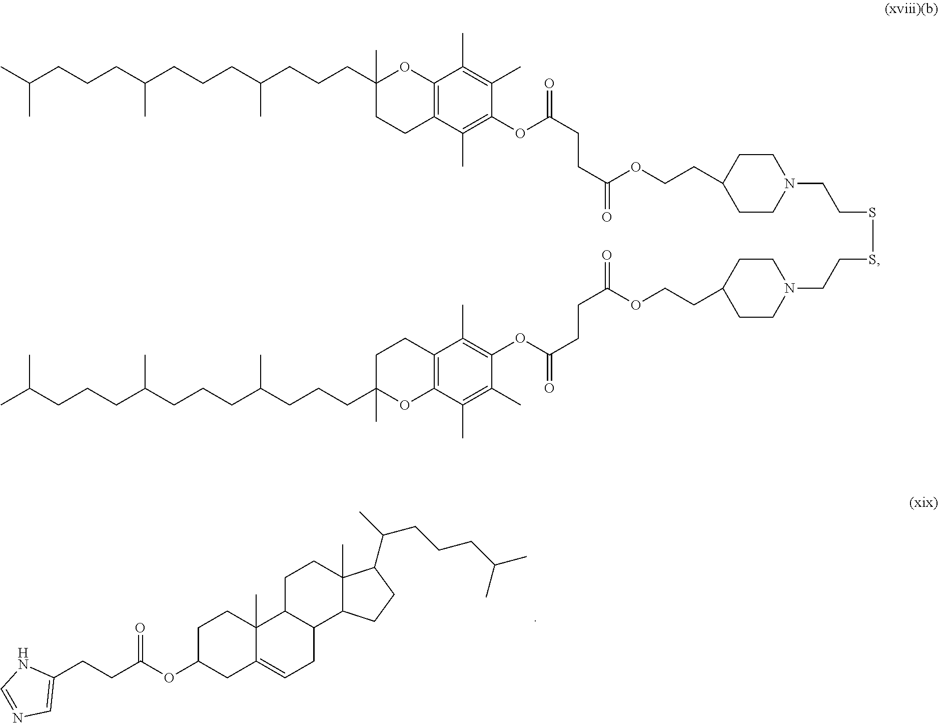 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2 ; CC(C)CCCC(C)C1CCC2C3CC=C4CC(OC(=O)CCc5cnc[nH]5)CCC4(C)C3CCC12C]

In some embodiments, the lipid composition further comprises one or more compounds of formula (xi), (xii), (xiii), (xiv), (xv), (xvi), (xvii), (xviii) (e.g., (xviii)a, (xviii)b), or (xix).

In some embodiments, the lipid composition further comprises lipids formed by one of the following reactions:

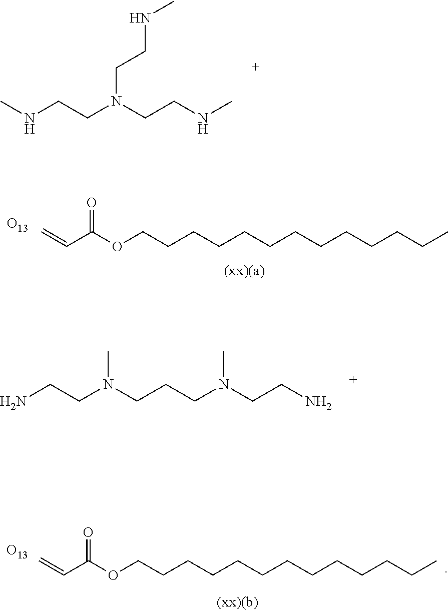

In some embodiments, the lipid composition further comprises the lipid (e.g., in combination with the lipid compounds described herein and other lipid components) having the formula (xxi):

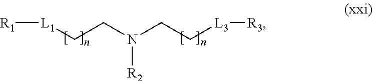

wherein:
each n is independently an integer from 2-15;
L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkyl sulfonyl, and alkyl sulfonealkyl; and
R₂ is selected from a group consisting of:

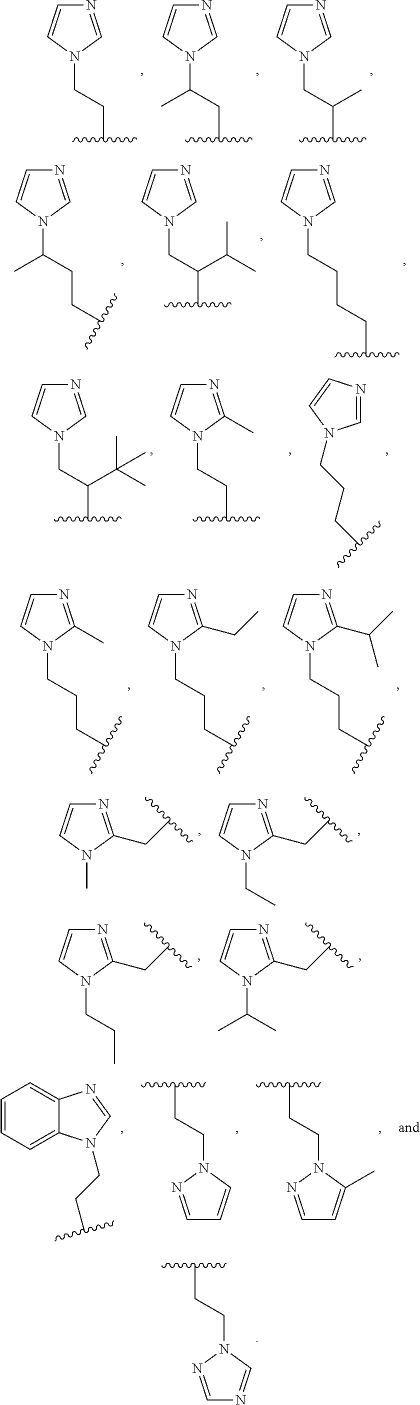

In some embodiments, the lipid composition further comprises one or more compounds of formula (xxi). In some embodiments, the compounds of formula (xxi) include those described by WO 2021/113777 (e.g., a lipid of Formula (1) such as a lipid of Table 1 of WO 2021/113777), which is incorporated herein by reference in its entirety.

In some embodiments, the lipid composition further comprises lipids (e.g., in combination with the lipid compound described herein and other lipid components) having the formula (xxii):

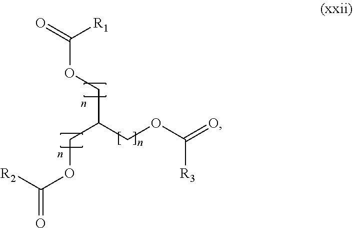

wherein:
each n is independently an integer from 1-15;
R¹ and R₂ are each independently selected from a group consisting of:

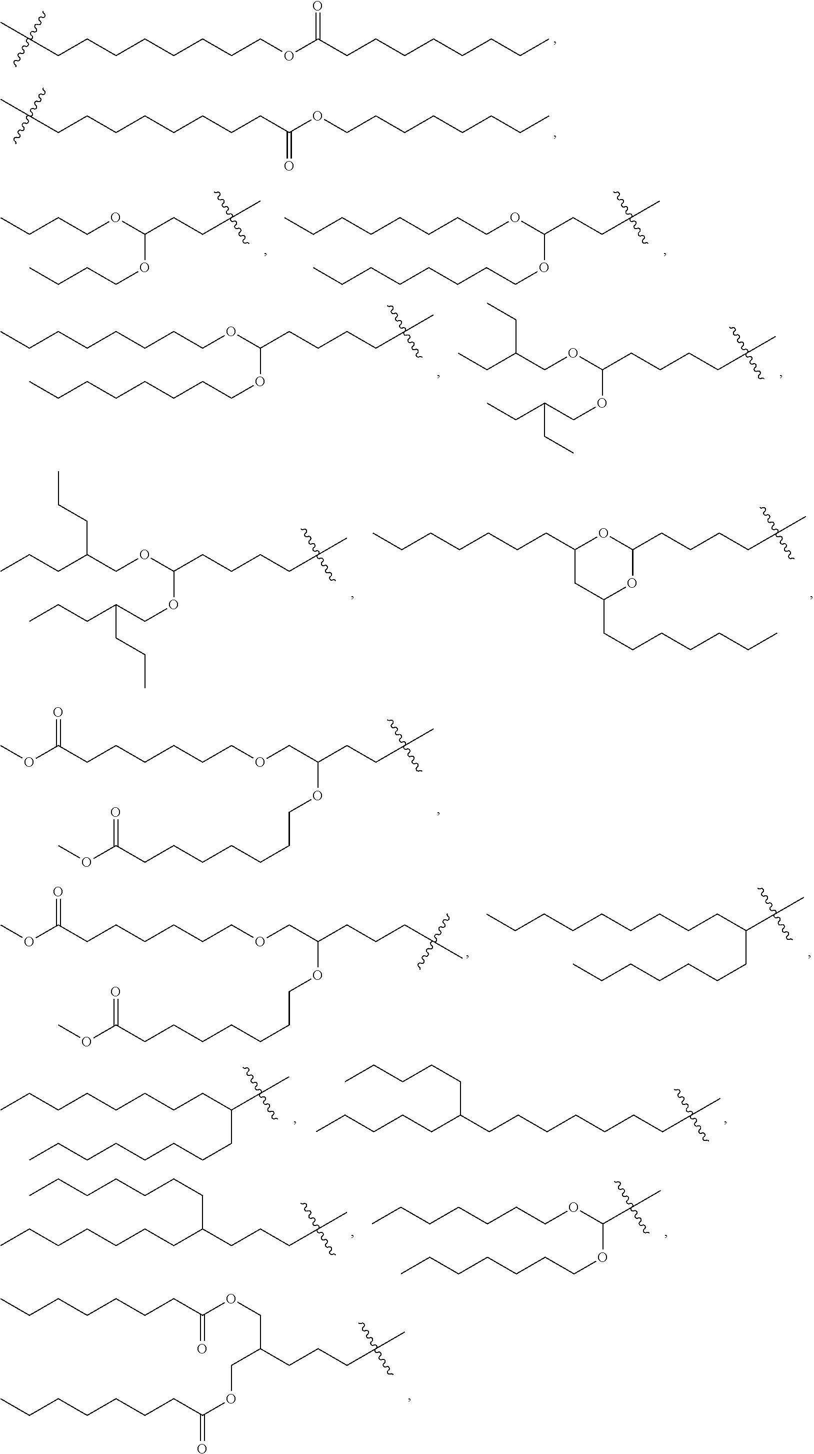

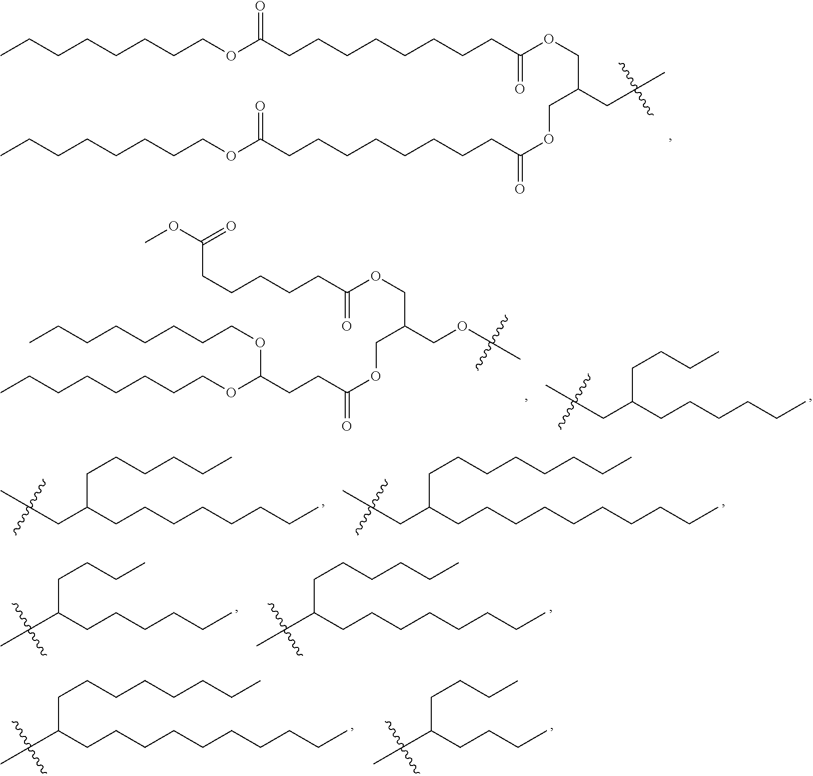

R³ is selected from a group consisting of:

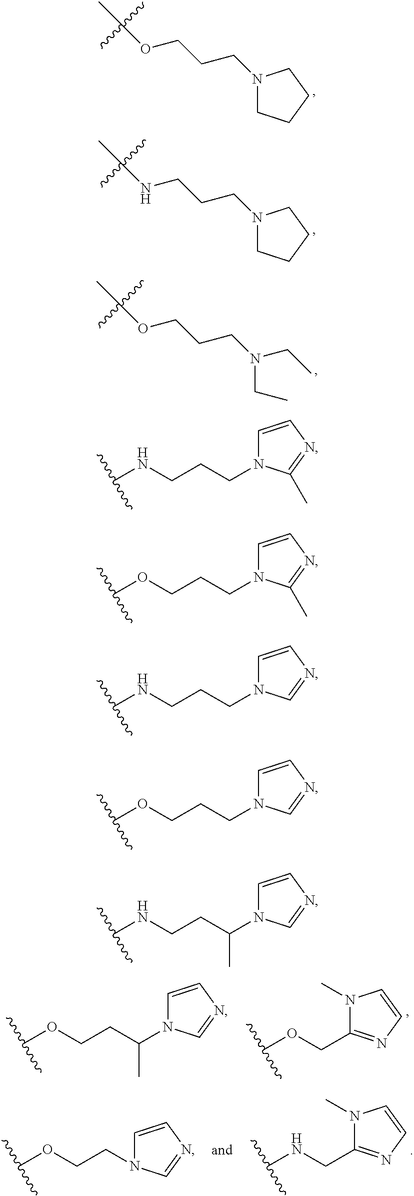

In some embodiments, the lipid composition further comprises one or more compounds of formula (xxii). In some embodiments, the compounds of formula (xxii) include those described by WO 2021/113777 (e.g., a lipid of Formula (2) such as a lipid of Table 2 of WO 2021/113777), which is incorporated herein by reference in its entirety.

In some embodiments, the lipid composition further comprises lipids (e.g., in combination with the lipid compound described herein and other lipid components) having the formula (xxiii):

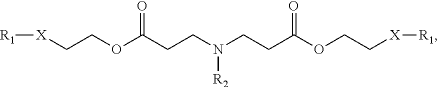

wherein
X is selected from —O—, —S—, or —OC(O)—*, wherein * indicates the attachment point to R₁;
R₁ is selected from a group consisting of:

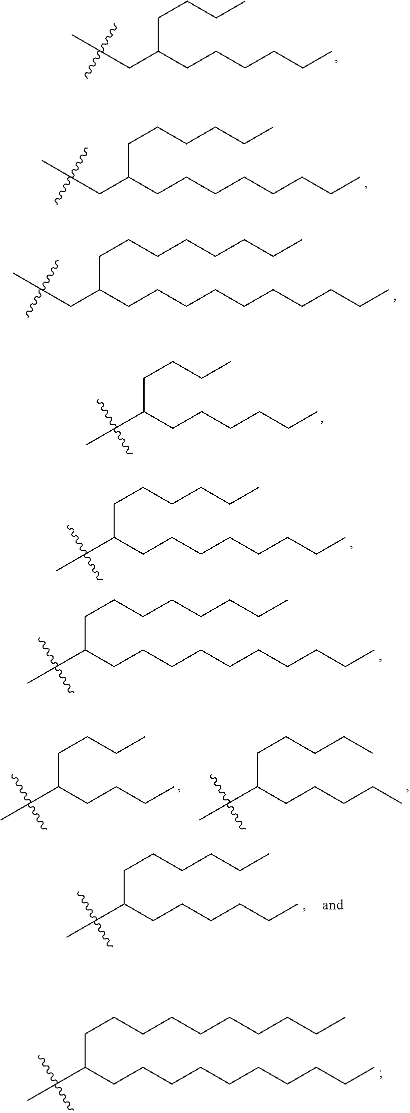

and
R₂ is selected from a group consisting of:

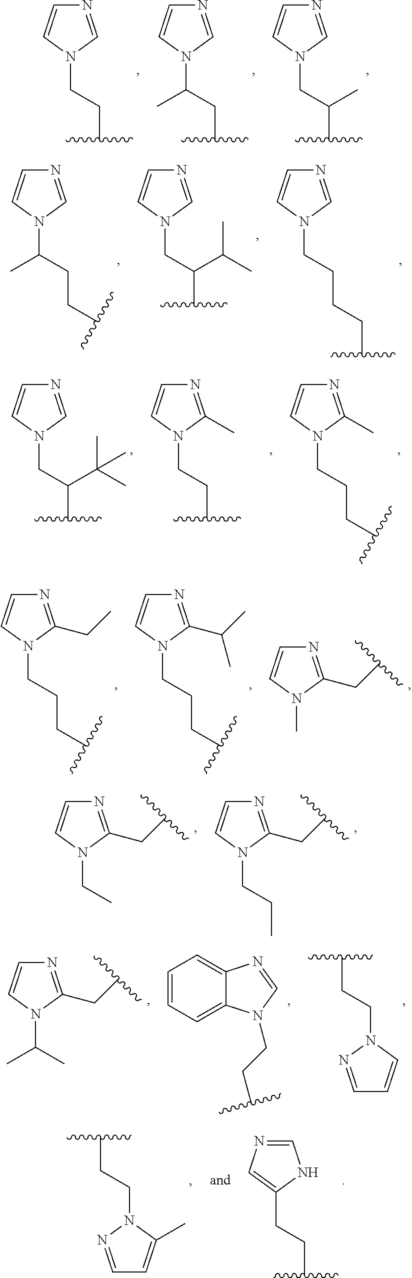

In some embodiments, the lipid composition further comprises one or more compounds of formula (xxiii). In some embodiments, the compounds of formula (xxiii) include those described by WO 2021/113777 (e.g., a lipid of Formula (3) such as a lipid of Table 3 of WO 2021/113777), which is incorporated herein by reference in its entirety.

Examples of additional lipids that can be used in the lipid composition include, without limitation, one or more of the following formulas: X of US 2016/0311759; I of US 20150376115 or in US 2016/0376224; I, II or III of US 2016/0151284; I, IA, II, or IIA of US 2017/0210967; I-c of US 2015/0140070; A of US 2013/0178541; I of US 2013/0303587 or US 2013/0123338; I of US 2015/0141678; II, III, IV, or V of US 2015/0239926; I of US 2017/0119904; I or II of WO 2017/117528; A of US 2012/0149894; A of US 2015/0057373; A of WO 2013/116126; A of US 2013/0090372; A of US 2013/0274523; A of US 2013/0274504; A of US 2013/0053572; A of WO 2013/016058; A of WO 2012/162210; I of US 2008/042973; I, II, III, or IV of US 2012/01287670; I or II of US 2014/0200257; I, II, or III of US 2015/0203446; I or III of US 2015/0005363; I, IA, IB, IC, ID, II, IIA, IIB, IIC, IID, or III-XXIV of US 2014/0308304; of US 2013/0338210; I, II, III, or IV of WO 2009/132131; A of US 2012/01011478; I or XXXV of US 2012/0027796; XIV or XVII of US 2012/0058144; of US 2013/0323269; I of US 2011/0117125; I, II, or III of US 2011/0256175; I, II, III, IV, V, VI, VII, VIII, IX, X, XI, XII of US 2012/0202871; I, II, III, IV, V, VI, VII, VIII, X, XII, XIII, XIV, XV, or XVI of US 2011/0076335; I or II of US 2006/008378; I of US 2013/0123338; I or X-A-Y—Z of US 2015/0064242; XVI, XVII, or XVIII of US 2013/0022649; I, II, or III of US 2013/0116307; I, II, or III of US 2013/0116307; I or II of US 2010/0062967; I-X of US 2013/0189351; I of US 2014/0039032; V of US 2018/0028664; I of US 2016/0317458; I of US 2013/0195920; 5, 6, or 10 of U.S. Pat. No. 10,221,127; III-3 of WO 2018/081480; I-5 or I-8 of WO 2020/081938; 18 or 25 of U.S. Pat. No. 9,867,888; A of US 2019/0136231; II of WO 2020/219876; 1 of US 2012/0027803; OF-02 of US 2019/0240349; 23 of U.S. Pat. No. 10,086,013; cKK-E12/A6 of Miao et al (2020); C12-200 of WO 2010/053572; 7C₁ of Dahlman et al (2017); 304-O13 or 503-O13 of Whitehead et al; TS-P4C2 of U.S. Pat. No. 9,708,628; I of WO 2020/106946; I of WO 2020/106946; (1), (2), (3), or (4) of WO 2021/113777; and any one of Tables 1-16 of WO 2021/113777, all of which are incorporated herein by reference in their entirety.

In some embodiments, the lipid conjugate (e.g., PEG-lipid) is present from 0.1 mol % to 2 mol %, from 0.5 mol % to 2 mol %, from 1 mol % to 2 mol %, from 0.6 mol % to 1.9 mol %, from 0.7 mol % to 1.8 mol %, from 0.8 mol % to 1.7 mol %, from 0.9 mol % to 1.6 mol %, from 0.9 mol % to 1.8 mol %, from 1 mol % to 1.8 mol %, from 1 mol % to 1.7 mol %, from 1.2 mol % to 1.8 mol %, from 1.2 mol % to 1.7 mol %, from 1.3 mol % to 1.6 mol %, or from 1.4 mol % to 1.5 mol % (or any fraction thereof or range therein) of the total lipids present in the lipid composition. In some embodiments, the lipid conjugate (e.g., PEG-lipid) is present from 0 mol % to 20 mol %, from 0.5 mol % to 20 mol %, from 2 mol % to 20 mol %, from 1.5 mol % to 18 mol %, from 2 mol % to 15 mol %, from 4 mol % to 15 mol %, from 2 mol % to 12 mol %, from 5 mol % to 12 mol %, or 2 mol % (or any fraction thereof or range therein) of the total lipids present in the lipid composition.

In some embodiments, the lipid conjugate (e.g., PEG-lipid) lipid composition from 4 mol % to 10 mol %, from 5 mol % to 10 mol %, from 5 mol % to 9 mol %, from 5 mol % to 8 mol %, from 6 mol % to 9 mol %, from 6 mol % to 8 mol %, or 5 mol %, 6 mol %, 7 mol %, 8 mol %, 9 mol %, or 10 mol % (or any fraction thereof or range therein) of the total lipids present in the lipid composition.

The percentage of lipid conjugate (e.g., PEG-lipid) present in the lipid composition of the disclosure is a target amount, and the actual amount of lipid conjugate present in the composition may vary, for example, by ±2 mol %. One of ordinary skill in the art will appreciate that the concentration of the lipid conjugate can be varied depending on the lipid conjugate employed and the rate at which the lipid particle is to become fusogenic.

By controlling the composition and concentration of the lipid conjugate, one can control the rate at which the lipid conjugate exchanges out of the lipid composition and, in turn, the rate at which the lipid composition becomes fusogenic. In addition, other variables including, e.g., pH, temperature, or ionic strength, can be used to vary and/or control the rate at which the lipid composition becomes fusogenic. Other methods which can be used to control the rate at which the lipid composition becomes fusogenic will become apparent to those of skill in the art upon reading this disclosure. Also, by controlling the composition and concentration of the lipid conjugate, one can control the lipid particle size.

In some embodiments, the lipid composition containing a ionizable lipid compound may comprise 30-70% ionizable lipid compound, 0-60% cholesterol, 0-30% phospholipid and 1-10% polyethylene glycol (PEG). In some embodiments, the lipid composition comprises 30-40% ionizable lipid compound, 40-50% cholesterol, and 10-20% PEG. In some embodiments, the lipid composition comprises 50-75% ionizable lipid compound, 20-40% cholesterol, and 5-10% phospholipid, and 1-10% PEG. The lipid composition may contain 60-70% ionizable lipid compound, 25-35% cholesterol, and 5-10% PEG-lipid.

In some embodiments, the lipid component of the lipid composition includes about 30 mol % to about 60 mol % (e.g., about 35-55 mol %, or about 40-50 mol %) an ionizable lipid compound as described herein, about 0 mol % to about 30 mol % (e.g., 5-25 mol %, or 10-20 mol %) phospholipid, about 15 mol % to about 50 mol % (e.g., 18.5-48.5 mol %, or 30-40 mol %) sterol, and about 0 mol % to about 10 mol % (e.g., 1-5 mol %, or 1.5-2.5 mol %) PEGylated lipid, provided that the total mol % of the lipid component does not exceed 100%.

In some embodiments, the lipid composition may contain up to 90% ionizable lipid compound and 2-15% helper lipid.

In some embodiments, the lipid composition may be a lipid particle composition, for example containing 8-30% ionizable lipid compound, 5-30% helper lipid, and 0-20% cholesterol. In some embodiments, the lipid nanoparticle composition contains 4-25% ionizable lipid, 4-25% helper lipid, 2-25% cholesterol, 10-35% cholesterol-PEG, and 5% cholesterol-amine. In some embodiments, the lipid nanoparticle composition contains 2-30% ionizable lipid, 2-30% helper lipid, 1-15% cholesterol, 2-35% cholesterol-PEG, and 1-20% cholesterol-amine. In some embodiments, the lipid nanoparticle composition contains up to 90% ionizable lipid and ²-10% helper lipids. In some embodiments, the lipid nanoparticle composition contains 100% ionizable lipids.

### Other Components for the LNP Composition

The lipid nanoparticle composition may include one or more components in addition to those described above. For example, a LN P composition may include one or more small hydrophobic molecules such as a vitamin (e.g., vitamin A or vitamin E) or a sterol.

The lipid nanoparticle composition may also include one or more permeability enhancer molecules, carbohydrates, polymers, surface altering agents, or other components.

Suitable carbohydrates may include simple sugars (e.g., glucose) and polysaccharides (e.g., glycogen and derivatives and analogs thereof).

A polymer may be used to encapsulate or partially encapsulate a nanoparticle composition. The polymer may be biodegradable and/or biocompatible. Suitable polymers include, but are not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyarylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Suitable surface altering agents include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a lipid nanoparticle and/or on the surface of a lipid nanoparticle (e.g., by coating, adsorption, covalent linkage, or other process).

The lipid nanoparticle composition nay also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a lipid nanoparticle may also be conjugated with one or more useful antibodies. Functional groups and conjugates useful in targeted cell delivery, imaging, and membrane permeation are well known in the art.

The lipid nanoparticle composition may include any substance useful in pharmaceutical compositions. For example, the lipid nanoparticle composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included.

Suitable diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Suitable surface active agents and/or emulsifiers may include, but are not limited to, natural emulsifiers (e, acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxydimethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

Suitable binding agents may be starch (e.g. cornstarch and starch paste); gelatin; sugars (e.g. sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Suitable preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Suitable lubricating agents include, but are not limited to, magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Suitable oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice brain, rosemary, safflower, sandalwood, sasquana, savoury, sea buckthorn, sesame, shea butter, silicone, soybean, sunflower, tea tree, thistle, tsubaki, vetiver, walnut, and wheat ern oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

In some embodiments, the lipid composition further comprises one or more cryoprotectants. Suitable cryoprotective agents include, but are not limited to, a polyol (e.g., a diol or a triol such as propylene glycol (i.e., 1,2-propanediol, 1,3-propanediol, glycerol, (+/−)-2-methyl-2,4-pentanediol, 1,6-hexanediol, 1,2-butanediol, 2,3-butanediol, ethylene glycol, or diethylene glycol), a nondetergent sulfobetaine e.g. NDSB-201 (3-(1-pyridino)-1-propane sulfonate), an osmolyte (e.g., L-proline or trimethylamine N-oxide dihydrate), a polymer (e.g., polyethylene glycol 200 (PEG 200), PEG 400, PEG 600, PEG 1000, PEG₂ₖ-DMG, PEG 3350, PEG 4000, PEG 8000, PEG 10000, PEG 20000, polyethylene glycol monomethyl ether 550 (mPEG 550), mPEG 600, mPEG 2000, mPEG 3350, mPEG 4000, mPEG 5000, polyvinylpyrrolidone (e.g., polyvinylpyrrolidone K 15), pentaerythritol propoxylate, or polypropylene glycol P 400), an organic solvent (e.g., dimethyl sulfoxide (DMSO) or ethanol), a sugar e D-(+)-sucrose, D-sorbitol trehalose, D-(+)-maltose monohydrate, meso-erythritol, xylitol, myo-inositol, D-(+)-raffinose pentahydrate, D-(+)-trehalose dihydrate, or D-(+)-glucose monohydrate), or a salt (e.g., lithium acetate, lithium chloride, lithium formate, lithium nitrate, lithium sulfate, magnesium acetate, sodium acetate, sodium chloride, sodium formate, sodium malonate, sodium nitrate, sodium sulfate, or any hydrate thereof), or any combination thereof.

In some embodiments, the cryoprotectant comprises sucrose. In some embodiments, the cryoprotectant and/or excipient is sucrose. In some embodiments, the cryoprotectant comprises sodium acetate. In some embodiments, the cryoprotectant and/or excipient is sodium acetate. In some embodiments, the cryoprotectant comprises sucrose and sodium acetate.

In some embodiments, the composition further comprises one or more buffers. Suitable buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof.

In some embodiments, the buffer is an acetate buffer, a citrate buffer, a phosphate buffer, a tris buffer, or combinations thereof.

In some embodiments, the lipid composition further comprises one or more nucleic acids, ionizable lipids, amphiphiles, phospholipids, cholesterol, and/or PEG-linked cholesterol.

### Pharmaceutical Compositions

Another aspect of the disclosure also provides pharmaceutical compositions comprising the lipid composition as described herein, which comprises one or more lipid compounds chosen from an ionizable lipid compound described herein (e.g., those having a structure of formula (I), (IA-1), (IA-2), (IIA)-(IIC), (IIA-1), (IIIA)-(IIIIE), (IIIC-1), (IVA-1)-(IVA-3), (IVC-1)-(IVC-2), and (VC-1)-(VC-6), and a pharmaceutically acceptable excipient. The pharmaceutical composition may further comprise a therapeutic agent.

All above descriptions and all embodiments discussed in the above aspects relating to the aspects of the lipid compounds, including the compounds covered by formulas (I), (IA-1), (IA-2), (IIA)-(IIC), (IIA-1), (IIIA)-(IIIIE), (IIIC-1), (IVA-1)-(IVA-3), (IVC-1)-(IVC-2), and (VC-1)-(VC-6), and the exemplary variables and compounds are all applicable to these aspects of the invention relating to the pharmaceutical composition.

All above descriptions and all embodiments discussed in the above aspects relating to the aspects of the lipid composition, including various other lipid components, are applicable to these aspects of the invention relating to the pharmaceutical composition.

In the lipid composition containing the therapeutic agent, the ratio of total lipid components to the cargo (e.g., an encapsulated therapeutic agent such as a nucleic acid) can be varied as desired. For example, the total lipid components to the cargo (mass or weight) ratio can be from about 10:1 to about 30:1. In some embodiments, the total lipid components to the cargo ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid components and the cargo can be adjusted to provide a desired N/P ratio, for example, N/P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the lipid composition's overall lipid content can range from about 5 mg/ml to about 30 mg/mL.

### Therapeutic Agents

### Nucleic Acid Molecule

In some embodiments, the lipid composition further comprises one or more nucleic acid molecule. The nucleic acid molecule may be a plasmid, an immunostimulatory oligonucleotide, an antisense oligonucleotide, an antagomir, an aptamer, a deoxyribozyme (DNAzyme), and a ribozyme.

In some embodiments, the lipid composition further comprises one or more RNA and/or DNA.

In some embodiments, the nucleic acid molecule is DNA. In some embodiments, the DNA is linear DNA, circular DNA, single stranded DNA, or double stranded DNA.

In some embodiments, the nucleic acid molecule is RNA. In some embodiments, the RNA is mRNA, miRNA, siRNA, RNA aptamer, linear RNA, circular RNA, single stranded RNA, double stranded RNA, tRNA, microRNA (miRNA) or miRNA precursor, a Dicer substrate small interfering RNA (dsiRNA), a short hairpin RNA (shRNA), an asymmetric interfering RNA (aiRNA), a guide RNA (gRNA), lncRNA, ncRNA, sncRNA, rRNA, snRNA, piRNA, snoRNA, snRNA, scaRNA, exRNA, scaRNA, Y RNA, or hnRNA. In some embodiments, the RNA is mRNA. In one embodiment, the mRNA is a modified mRNA.

In some embodiments, the nucleic acid molecule is an enzymatic nucleic acid molecule. The term “enzymatic nucleic acid molecule” refers to a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity.

In some embodiments, the nucleic acid molecule is an antisense nucleic acid. The term “antisense nucleic acid” refers to a non-enzymatic nucleic acid molecule that binds to target RNA by means of RNA-RNA or RNA-DNA or RNA-PNA (protein nucleic acid) interactions and alters the activity of the target RNA.

In some embodiments, the nucleic acid molecule may be a 2-5A antisense chimera. The term “2-5A antisense chimera” refers to an antisense oligonucleotide containing a 5′-phosphorylated 2′-5′-linked adenylate residue.

In some embodiments, the nucleic acid molecule may be a triplex forming oligonucleotide. The term “triplex forming oligonucleotide” refers to an oligonucleotide that can bind to a double-stranded DNA in a sequence-specific manner to form a triple-strand helix.

In some embodiments, the nucleic acid molecule may be a decoy RNA. The term “decoy RNA” refers to a RNA molecule or aptamer that is designed to preferentially bind to a predetermined ligand. Such binding can result in the inhibition or activation of a target molecule.

In some embodiments, the nucleic acid molecule (e.g., RNA or DNA) encodes a therapeutic peptide or polypeptide, operably linked to a promoter for a DNA. The therapeutic peptide or polypeptide may be, e.g., a transcription factor; a chromatin remodeling factor; an antigen; a hormone; an enzyme (such as a nuclease, e.g., an endonuclease, e.g., a nuclease element of a CRISPR system, e.g., a Cas9, dCas9, aCas9-nickase, Cpf/Cas12a); a Crispr-linked enzyme, e.g., a base editor or prime editor; a mobile genetic element protein (e.g., a transposase, a retrotransposase, a recombinase, an integrase); a Gene Writer; a polymerase; a methylase; a demethylase; an acetylase; a deacetylase; a kinase; a phosphatase; a ligase; a deubiquitinase; an integrase; a recombinase; a topoisomerase; a gyrase; a helicase; a lysosomal acid hydrolase); an antibody; a receptor ligand; a receptor; a clotting factor; a membrane protein; a mitochondrial protein; a nuclear protein; an antibody or other protein scaffold binder such as a centyrin, darpin, or adnectin.

In some embodiments, the nucleic acid molecule is RNA comprising a gRNA nucleic acid. In some embodiments, the gRNA nucleic acid is a gRNA. In some embodiments, the nucleic acid molecule is RNA comprising a Class 2 Cas nuclease mRNA and a gRNA. In some embodiments, the gRNA nucleic acid is or encodes a dual-guide RNA (dgRNA). In some embodiments, the gRNA nucleic acid is or encodes a single-guide RNA (sgRNA). In some embodiments, the gRNA is a modified gRNA. In some embodiments, the modified gRNA comprises a modification at one or more of the first five nucleotides at a 5′ end. In some embodiments, the modified gRNA comprises a modification at one or more of the last five nucleotides at a 3′ end.

In some embodiments, the nucleic acid molecule is RNA comprising an mRNA. In some embodiments, the RNA components comprise an RNA-guided DNA-binding agent, for example a Cas nuclease mRNA (such as a Class 2 Cas nuclease mRNA) or a Cas9 nuclease mRNA.

All the nucleic acid molecules described herein can be chemically modified. The various modification strategy to the nucleic acid molecules are well known to one skilled in the art. In some embodiments, the nucleic acid molecule comprises one or more modifications selected from the group consisting of pseudouridine, 5-bromouracil, 5-methylcytosine, peptide nucleic acid, xeno nucleic acid, morpholinos, locked nucleic acids, glycol nucleic acids, threose nucleic acids, dideoxynucleotides, cordycepin, 7-deaza-GTP, florophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. In some embodiments, the antisense oligonucleotide may be a locked nucleic acid oligonucleotide (LNA). The term “locked nucleic acid (LNA)” refers to oligonucleotides that contain one or more nucleotide building blocks in which an extra methylene bridge fixes the ribose moiety either in the C3′-endo (beta-D-LNA) or C2′-endo (alpha-L-LNA) conformation (Grunweller A, Hartmann R K, BioDrugs, 21(4): 235-243 (2007)).

In some embodiments, the composition further comprises one or more template nucleic acids.

Additional examples of the nucleic acid molecules (including tumor suppressor genes, antisense oligonucleotides, siRNA, miRNA, or shRNA) may be found in U.S. Published Patent Application No. 2007/0065499 and U.S. Pat. No. 7,780,882, which are incorporated by reference herein in their entireties.

In some embodiments, the pharmaceutical composition can include a plurality of nucleic acid molecules, which may be the same or different types.

Nucleic acids for use with embodiments of this disclosure may be prepared according to any available technique. For mRNA, the primary methodology of preparation is, but not limited to, enzymatic synthesis (also termed in vitro transcription) which currently represents the most efficient method to produce long sequence-specific mRNA. In vitro transcription describes a process of template-directed synthesis of RNA molecules from an engineered DNA template comprised of an upstream bacteriophage promoter sequence (e.g., including but not limited to that from the T7, T3 and SP6 coliphage) linked to a downstream sequence encoding the gene of interest. Template DNA can be prepared for in vitro transcription from a number of sources with appropriate techniques which are well known in the art including, but not limited to, plasmid DNA and polymerase chain reaction amplification (see Linpinsel, J. L and Conn, G. L., General protocols for preparation of plasmid DNA template and Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012).

Transcription of the RNA occurs in vitro using the linearized DNA template in the presence of the corresponding RNA polymerase and adenosine, guanosine, uridine and cytidine ribonucleoside triphosphates (rNTPs) under conditions that support polymerase activity while minimizing potential degradation of the resultant mRNA transcripts. In vitro transcription can be performed using a variety of commercially available kits including, but not limited to RiboMax Large Scale RNA Production System (Promega), MegaScript Transcription kits (Life Technologies) as well as with commercially available reagents including RNA polymerases and rNTPs. The methodology for in vitro transcription of mRNA is well known in the art. (see, e.g. Losick, R., 1972, In vitro transcription, Ann Rev Biochem v.41 409-46; Kamakaka, R. T. and Kraus, W. L. 2001. In Vitro Transcription. Current Protocols in Cell Biology. 2: 11.6:11.6.1-11.6.17; Beckert, B. And Masquida, B., (2010) Synthesis of RNA by In Vitro Transcription in RNA in Methods in Molecular Biology v. 703 (Neilson, H. Ed), New York, N.Y. Humana Press, 2010; Brunelle, J. L. and Green, R., 2013, Chapter Five—In vitro transcription from plasmid or PCR-amplified DNA, Methods in Enzymology v. 530, 101-114; all of which are incorporated herein by reference).

The desired in vitro transcribed mRNA may be purified from the undesired components of the transcription or associated reactions (including unincorporated rNTPs, protein enzyme, salts, short RNA oligos, etc.). Techniques for the isolation of the mRNA transcripts are well known in the art. Well known procedures include, for non-limiting examples, phenol/chloroform extraction or precipitation with either alcohol (ethanol, isopropanol) in the presence of monovalent cations or lithium chloride.

Additional, non-limiting examples of purification procedures which can be used include size exclusion chromatography (Lukavsky, P. J. and Puglisi, J. D., 2004, Large-scale preparation and purification of polyacrylamide-free RNA oligonucleotides, RNA v.10, 889-893), silica-based affinity chromatography and polyacrylamide gel electrophoresis (Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012). Purification can be performed using a variety of commercially available kits including, but not limited to SV Total Isolation System (Promega) and In Vitro Transcription Cleanup and Concentration Kit (Norgen Biotek).

Furthermore, while reverse transcription can yield large quantities of mRNA, the products can contain a number of aberrant RNA impurities associated with undesired polymerase activity which may need to be removed from the full-length mRNA preparation. These include short RNAs that result from abortive transcription initiation as well as double-stranded RNA (dsRNA) generated by RNA-dependent RNA polymerase activity, RNA-primed transcription from RNA templates and self-complementary 3′ extension. It has been demonstrated that these contaminants with dsRNA structures can lead to undesired immunostimulatory activity through interaction with various innate immune sensors in eukaryotic cells that function to recognize specific nucleic acid structures and induce potent immune responses. This in turn, can dramatically reduce mRNA translation since protein synthesis is reduced during the innate cellular immune response. Therefore, additional techniques to remove these dsRNA contaminants have been developed and are known in the art including but not limited to scaleable HPLC purification (see, e.g., Kariko, K., Muramatsu, H., Ludwig, J. And Weissman, D., 2011, Generating the optimal mRNA for therapy: HPLC purification eliminates immune activation and improves translation of nucleoside-modified, protein-encoding mRNA, Nucl Acid Res, v. 39 el42; Weissman, D., Pardi, N., Muramatsu, H., and Kariko, K., HPLC Purification of in vitro transcribed long RNA in Synthetic Messenger RNA and Cell Metabolism Modulation in Methods in Molecular Biology v.969 (Rabinovich, P. H. Ed), 2013). HPLC purified mRNA has been reported to be translated at much greater levels, particularly in primary cells and in vivo.

A significant variety of modifications have been described in the art which are used to alter specific properties of in vitro transcribed mRNA, and may improve its utility. These include, but are not limited to modifications to the 5′ and 3′ termini of the mRNA. Endogenous eukaryotic m RNA typically contain a cap structure on the 5′-end of a mature molecule which plays an important role in mediating binding of the mRNA Cap Binding Protein (CBP), which is in turn responsible for enhancing mRNA stability in the cell and efficiency of mRNA translation. Therefore, highest levels of protein expression are achieved with capped mRNA transcripts. The 5′-cap contains a 5′-5′-triphosphate linkage between the 5′-most nucleotide and guanine nucleotide. The conjugated guanine nucleotide is methylated at the N7 position. Additional modifications include methylation of the ultimate and penultimate most 5′-nucleotides on the 2′-hydroxyl group.

Multiple distinct cap structures can be used to generate the 5′-cap of in vitro transcribed synthetic mRNA. 5′-capping of synthetic mRNA can be performed co-transcriptionally with chemical cap analogs (i.e., capping during in vitro transcription). For example, the Anti-Reverse Cap Analog (ARC A) cap contains a 5′-5′-triphosphate guanine-guanine linkage where one guanine contains an N7 methyl group as well as a Y—O-methyl group. However, up to 20% of transcripts remain uncapped during this co-transcriptional process and the synthetic cap analog is not identical to the 5′-cap structure of an authentic cellular mRNA, potentially reducing translatability and cellular stability. Alternatively, synthetic mRNA molecules may also be enzymatically capped post-transcriptionally. These may generate a more authentic 5′-cap structure that more closely mimics, either structurally or functionally, the endogenous 5′-cap which have enhanced binding of cap binding proteins, increased half-life and reduced susceptibility to 5′ endonucleases and/or reduced 5′ decapping. Numerous synthetic 5′-cap analogs have been developed and are known in the art to enhance mRNA stability and translatability (see, e.g., Grudzien-Nogalska, E., Kowalska, J., Su, W., Kuhn, A. N., Slepenkov, S. V., Darynkiewicz, E., Sahin, U., Jemielity, J., and Rhoads, R. E., Synthetic mRNAs with superior translation and stability properties in Synthetic Messenger RNA and Cell Metabolism Modulation in Methods in Molecular Biology v.969 (Rabinovich, P. H. Ed), 2013).

On the 3′-terminus, a long chain of adenine nucleotides (poly-A tail) is normally added to mRNA molecules during RNA processing. Immediately after transcription, the 3′ end of the transcript is cleaved to free a 3′ hydroxyl to which poly-A polymerase adds a chain of adenine nucleotides to the RNA in a process called polyadenylation. The poly-A tail has been extensively shown to enhance both translational efficiency and stability of mRNA (see Bernstein, P. and Ross, J., 1989, Poly (A), poly (A) binding protein and the regulation of mPNA stability, Trends Bio Sci v. 14 373-377; Guhaniyogi. J. And Brewer, G., 2001, Regulation of mRNA stability in mammalian cells, Gene, v. 265, 11-23; Dreyfus, M. And Regnier, P., 2002, The poly (A) tail of mRNAs: Bodyguard in eukaryotes, scavenger in bacteria, Cell, v. I1, 611-613).

Poly (A) tailing of in vitro transcribed mRNA can be achieved using various approaches including, but not limited to, cloning of a poly (T) tract into the DNA template or by post-transcriptional addition using Poly (A) polymerase. The first case allows in vitro transcription of mRNA with poly (A) tails of defined length, depending on the size of the poly (T) tract, but requires additional manipulation of the template. The latter case involves the enzymatic addition of a poly (A) tail to in vitro transcribed mRNA using poly (A) polymerase which catalyzes the incorporation of adenine residues onto the 3′-termini of RNA, requiring no additional manipulation of the DNA template, but results in mRNA with poly(A) tails of heterogeneous length. 5′-capping and 3′-poly (A) tailing can be performed using a variety of commercially available kits including, but not limited to Poly (A) Polymerase Tailing kit (EpiCenter), mMESSAGE mMACHINE T7 Ultra kit and Poly (A) Tailing kit (Life Technologies) as well as with commercially available reagents, various ARCA caps, Poly (A) polymerase, etc.

In addition to 5′ cap and 3′ poly adenylation, other modifications of the in vitro transcripts have been reported to provide benefits as related to efficiency of translation and stability. It is well known in the art that pathogenic DNA and RNA can be recognized by a variety of sensors within eukaryotes and trigger potent innate immune responses. The ability to discriminate between pathogenic and self DNA and RNA has been shown to be based, at least in part, on structure and nucleoside modifications since most nucleic acids from natural sources contain modified nucleosides. In contrast, in vitro synthesized RNA lacks these modifications, thus rendering it immunostimulatory which in turn can inhibit effective mRNA translation as outlined above. The introduction of modified nucleosides into in vitro transcribed mRNA can be used to prevent recognition and activation of RNA sensors, thus mitigating this undesired immunostimulatory activity and enhancing translation capacity (see, e.g., Kariko, K. And Weissman, D. 2007, Naturally occurring nucleoside modifications suppress the immunostimulatory activity of RNA: implication for therapeutic RNA development, Curr Opin Drug Discov Devel, v.10 523-532; Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v.969 (Rabinovich, P. H. Ed), 2013; Kariko, K., Muramatsu, H., Welsh, F. A., Ludwig, J., Kato, H., Akira, S., Weissman, D., 2008, Incorporation of Pseudouridine Into mRNA Yields Superior Nonimmunogenic Vector With Increased Translational Capacity and Biological Stability, Mol Ther v.16, 1833-1840). The modified nucleosides and nucleotides used in the synthesis of modified RNAs can be prepared monitored and utilized using general methods and procedures known in the art. A large variety of nucleoside modifications are available that may be incorporated alone or in combination with other modified nucleosides to some extent into the in vitro transcribed mRNA (see, e.g., US2012/0251618). In vitro synthesis of nucleoside-modified mRNA has been reported to have reduced ability to activate immune sensors with a concomitant enhanced translational capacity.

Other components of mRNA which can be modified to provide benefit in terms of translatability and stability include the 5′ and 3′ untranslated regions (UTR). Optimization of the UTRs (favorable 5′ and 3′ UTRs can be obtained from cellular or viral RNAs), either both or independently, have been shown to increase mRNA stability and translational efficiency of in vitro transcribed mRNA (see, e.g., Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v.969 (Rabinovich, P. H. Ed), 2013).

In addition to mRNA, other nucleic acid payloads may be used for this disclosure. For oligonucleotides, methods of preparation include but are not limited to chemical synthesis and enzymatic, chemical cleavage of a longer precursor, in vitro transcription as described above, etc. Methods of synthesizing DNA and RNA nucleotides are widely used and well known in the art (see, e.g., Gait, M. J. (ed.) Oligonucleotide synthesis: a practical approach, Oxford [Oxfordshire], Ishington, D. C.: IRL Press, 1984; and Herdewijn, P. (ed.) Oligonucleotide synthesis: methods and applications, Methods in Molecular Biology, v. 288 (Clifton, N. J.) Totowa, N. J.: Humana Press, 2005; both of which are incorporated herein by reference).

For plasmid DNA, preparation for use with embodiments of this disclosure commonly utilizes, but is not limited to, expansion and isolation of the plasmid DNA in vitro in a liquid culture of bacteria containing the plasmid of interest. The presence of a gene in the plasmid of interest that encodes resistance to a particular antibiotic (penicillin, kanamycin, etc.) allows those bacteria containing the plasmid of interest to selectively grow in antibiotic-containing cultures. Methods of isolating plasmid DNA are widely used and well known in the art (see, e.g., Heilig, J., Elbing, K. L. and Brent, R., (2001), Large-Scale Preparation of Plasmid DNA, Current Protocols in Molecular Biology, 41:11:1.7:1.7.1-1.7.16; Rozkov, A., Larsson, B., Gillstrom, S., Bjornestedt, R. and Schmidt, S. R., (2008), Large-scale production of endotoxin-free plasmids for transient expression in mammalian cell culture, Biotechnol. Bioeng., 99: 557-566; and U.S. Pat. No. 6,197,553 B1). Plasmid isolation can be performed using a variety of commercially available kits including, but not limited to Plasmid Plus (Qiagen), GenJET plasmid MaxiPrep (Thermo) and Pure Yield MaxiPrep (Promega) kits as well as with commercially available reagents.

In some embodiments, the lipid nanoparticle compositions are useful for expression of protein encoded by mRNA. In some embodiments, provided herein are methods for expression of protein encoded by mRNA.

In some embodiments, the lipid composition has an N/P ratio of from about 1:1 to about 30:1, for instance, from about 3:1 to about 20:1, from about 3:1 to about 15:1, from about 3:1 to about 10:1, or from about 3:1 to about 6:1. For example, the N/P ratio of the nucleic acid molecule-encapsulated lipid composition may be 6±1, or the N/P ratio of the nucleic acid molecule-encapsulated lipid composition may be 6±0.5. In some embodiments, the N/P ratio of the nucleic acid molecule—encapsulated lipid composition ranges from about 3:1 to about 15:1. In some embodiments, the N/P ratio of the nucleic acid molecule-encapsulated lipid composition is about 6. An N:P ratio refers to the molar ratio of the amines present in the lipid composition or lipid nanoformulation (e.g., the amines in the ionizable lipids) to the phosphates present in the nucleic acid molecule. It is a factor for efficient packaging and potency.

### Other Therapeutic Agents

The therapeutic agent can be a peptide or protein, a small molecule drug, encapsulated in the lipid composition. The pharmaceutical composition can contain two or more different therapeutic agents from the nucleic acid molecule, peptide or protein, and small molecule drug.

In some embodiments, the protein may be a peptide or polypeptide, e.g., a transcription factor; a chromatin remodeling factor; an antigen; a hormone; an enzyme (such as a nuclease, e.g., an endonuclease, e.g., a nuclease element of a CRISPR system, e.g., a Cas9, dCas9, aCas9-nickase, Cpf/Cas12a); a Crispr-linked enzyme, e.g., a base editor or prime editor; a mobile genetic element protein (e.g., a transposase, a retrotransposase, a recombinase, an integrase); a gene writer; a polymerase; a methylase; a demethylase; an acetylase; a deacetylase; a kinase; a phosphatase; a ligase; a deubiquitinase; an integrase; a recombinase; a topoisomerase; a gyrase; a helicase; a lysosomal acid hydrolase); an antibody; a receptor ligand; a receptor; a clotting factor; a membrane protein; a mitochondrial protein; a nuclear protein; an antibody or other protein scaffold binder such as a centyrin, darpin, or adnectin.

In some embodiments, the pharmaceutical composition can include a plurality of protein molecules, which may be the same or different types.

In some embodiments, the therapeutic agent is a small molecule drug, for instance, a small molecule drug approved for use in humans by an appropriate regulatory authority.

In some embodiments, the pharmaceutical composition can include a plurality of small molecule drugs, which may be the same or different types.

In some embodiments, the therapeutic agent is a vaccine. In some embodiments, the vaccine is a RNA vaccine, such as a RNA cancer vaccine or RNA vaccine for infectious disease (e.g., an influenza virus vaccine or a corona virus vaccine (e.g., COVID-19 vaccine).

### Other Ingredients

The pharmaceutical compositions may contain one or more pharmaceutically acceptable excipients. The pharmaceutically acceptable excipient is selected on the basis of the mode and route of administration. Suitable pharmaceutical carriers or excipients for use in pharmaceutical formulations are described in Remington: The Science and Practice of Pharmacy, 21^{st} Ed., Gennaro, Ed., Lippencott Williams & Wilkins (2005); Handbook of Pharmaceutical Excipients, 6^{th} Edition, Rowe et al., Eds., Pharmaceutical Press (2009); and the USP/NF (United States Pharmacopeia and the National Formulary), which are herein incorporated by reference in their entirety.

In some embodiments, the pharmaceutically acceptable excipient includes one or more of an antioxidant, binder, antiadherent, buffer, coloring agent, diluent (e.g., solid or liquid), disintegrant (e.g., coatings disintegrate), dispersing agent, dyestuff, filler, emulsifier, flavoring agent, lubricant, pH adjuster, pigment, preservative, stabilizer, solubilizing agent, solvent, suspending agent, sweetener, or wetting agent, or combination thereof.

Examples of suitable excipients include, without limitation, acacia, alginate, calcium phosphate, calcium carbonate, calcium silicate, carbopol gel, carboxymethyl cellulose, caranuba wax, cellulose, crospovidone, dextrose, diacetylated monoglycerides, ethylcellulose, gelatin, glyceryl monostearate 40-50, gum acacia, gum arabic, hydroxyethyl cellulose, hydroxypropyl cellulose, hydroxypropylmethyl cellulose, hypromellose phthalate, hypromellose, lactose, lecithin, magnesium stearate, kaolin, methacrylic acid copolymer type C, mannitol, methyl cellulose, methylhydroxybenzoate, microcrystalline cellulose, povidone, polyethylene glycol, polysorbate 80, polyvinylpyrrolidone, propylhydroxybenzoate, sodium carboxymethyl cellulose sodium hydroxide, sodium stearyl fumarate, sodium starch glycolate, starch, sorbitan monooleate sorbitol, sorbic acid, sucrose, talc, tragacanth, talc, triethyl citrate, titanium dioxide, yellow ferric oxide, talc, oil medium (e.g., peanut oil, liquid paraffin, mineral oil, olive oil, almond oil, glycerin, propylene glycol), or water, When the excipient serves as a diluent, it can be a solid, semisolid, or liquid material (e.g., normal saline), which acts as a vehicle, carrier or medium for the active ingredient. As is known in the art, the type of diluent can vary depending upon the intended route of administration.

The pharmaceutical compositions can comprise pharmaceutically acceptable carriers, excipients, or stabilizers in the form of lyophilized formulations or aqueous solutions. Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans: chelating agents such as EDTA; sugars such as sucrose, mannitol, trehalose or sorbitol; salt-forming counter-ions such as sodium; metal complexes (e.g., Zn— protein complexes); and/or nonionic surfactants such as TWEEN™, PLURONICS™ or polyethylene glycol (PEG).

Suitable carriers or excipients for the pharmaceutical compositions may also include a substance that enhances the ability of the body of an individual to absorb the LNP or liposome. Suitable carriers and/or excipients also include any substance that can be used to bulk up formulations with a LNP or liposome, to allow for convenient and accurate dosage. In addition, carriers and/or excipients may be used in the manufacturing process to aid in the handling of a LNP or liposome. Depending on the route of administration, and form of medication, different carriers and/or excipients may be used.

Carriers and/or excipients may also include vehicles and/or diluents. “Vehicles” indicates any of various media acting usually as solvents or carriers; “diluent” indicates a diluting agent which is issued to dilute an active ingredient of a composition; suitable diluent include any substance that can decrease the viscosity of a medicine. The type and amounts of carriers and/or excipient are chosen in function of the chosen pharmaceutical form; suitable pharmaceutical forms are liquid systems like solutions, infusions, suspensions; semisolid systems like colloids, gels, pastes or creams; solid systems like powders, granulates, tablets, capsules, pellets, microgranulates, minitablets, microcapsules, micropellets, suppositories; etc.

Each of the above systems can be suitably formulated for normal, delayed or accelerated release, using techniques well-known in the art.

### Formulations, Dosages, and Routes of Administration

The pharmaceutical compositions described herein can be prepared according to standard techniques, as well as those techniques described herein. For instance, the pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are known in the art. See, e.g., Remington: The Science and Practice of Pharmacy, 21^{st} Ed., Gennaro, Ed., Lippencott Williams & Wilkins (2005), and Encyclopedia of Pharmaceutical Technology, eds. J. Swarbrick and J. C. Boylan, 1988-1999, Marcel Dekker, New York.

The therapeutic agent may be encapsulated in the lipid composition, for instance, the therapeutic agent may be completely or partially located in the interior space of the LNPs, within the lipid layer/membrane, or associated with the exterior surface of the lipid layer/membrane. One purpose of incorporating therapeutic agents into LNPs is to protect the therapeutic agents from environments which may contain enzymes or chemicals or conditions that degrade the therapeutic agents and/or systems or receptors that cause the rapid excretion of the therapeutic agents. Moreover, incorporating therapeutic agents into LNPs may promote uptake of the therapeutic agent, and hence, may enhance the therapeutic effect.

In some embodiments, in the pharmaceutical composition, the lipid components to therapeutic agent ratio (mass/mass ratio; w/w ratio) can range from about 1:1 to about 25:1, 10:1 to about 14:1, about 3:1 to about 15:1, about 4:1 to about 10:1, about 5:1 to about 9:1, or about 6:1 to about 9:1.

The lipid composition or pharmaceutical composition may contain about 5 to about 95% by weight the therapeutic agent, based on the weight of the lipid composition or pharmaceutical composition. In some embodiments, the lipid composition or pharmaceutical composition contains about 5%, about 10%, about 20, about 30, about 40, about 50, about 60, about 70, about 80, about 90, or about 95% by weight, based on the weight of the LNP or pharmaceutical composition, of the therapeutic agent. In some embodiments, the lipid composition or pharmaceutical composition contains the therapeutic agent in an amount about 5-95%, about 5-90%, about 5-80%, about 5-70%, about 5-60%, about 5-50%, about 5-40%, about 5-30%, about 5-20%, about 5-10%, about 10-95%, about 10-90%, about 10-80%, about 10-70%, about 10-60%, about 10-50%, about 10-40%, about 10-30%, about 10-20%, about 20-95%, about 20-90%, about 20-80%, about 20-70%, about 20-60%, about 20-50%, about 20-40%, about 20-30%, about 30-95%, about 30-90%, about 30-80%, about 30-70%, about 30-60%, about 30-50%, about 30-40%, about 40-95%, about 40-90%, about 40-80%, about 40-70%, about 40-60%, about 40-50%, about 50-95%, about 50-90%, about 50-80%, about 50-70%, about 50-60%, about 60-95%, about 60-90%, about 60-80%, about 60-70%, about 70-95%, about 70-90%, about 70-80%, about 80-95%, about 80-90%, or about 90-95%, based on the weight of the lipid composition or pharmaceutical composition.

The lipid composition or pharmaceutical compositions can contain total lipids at an amount of about 5 to about 95% by weight, based on the weight of the lipid composition or pharmaceutical composition. In some embodiments, the lipid composition or pharmaceutical compositions contain total lipids at an amount of about 5-95%, about 5-90%, about 5-80%, about 5-70%, about 5-60%, about 5-50%, about 5-40%, about 5-30%, about 5-20%, about 5-10%, about 10-95%, about 10-90%, about 10-80%, about 10-70%, about 10-60%, about 10-50%, about 10-40%, about 10-30%, about 10-20%, about 20-95%, about 20-90%, about 20-80%, about 20-70%, about 20-60%, about 20-50%, about 20-40%, about 20-30%, about 30-95%, about 30-90%, about 30-80%, about 30-70%, about 30-60%, about 30-50%, about 30-40%, about 40-95%, about 40-90%, about 40-80%, about 40-70%, about 40-60%, about 40-50%, about 50-95%, about 50-90%, about 50-80%, about 50-70%, about 50-60%, about 60-95%, about 60-90%, about 60-80%, about 60-70%, about 70-95%, about 70-90%, about 70-80%, about 80-95%, about 80-90%, or about 90-95%, based on the weight of the lipid composition or pharmaceutical composition.

The lipid compositions or pharmaceutical compositions of this disclosure may be administered by various routes, for example, to effect systemic delivery via intravenous, parenteral, intraperitoneal, intramuscular, intracanalicular or topical routes. In some embodiments, a siRNA may be delivered intracellularly, for example, in cells of a target tissue such as lung or liver, or in inflamed tissues. In some embodiments, this disclosure provides a method for delivery of siRNA in vivo. A nucleic acid-lipid composition may be administered intravenously, subcutaneously, or intraperitoneally to a subject.

As used herein, the term “parenteral” refers to routes of administration aside from enteral administration. Examples of parenteral administration include, without limitation, buccal, epicutaneous, epidural, extra-amniotic, intra-arterial, intra-articular, intracardiac, intracavernous, intracerebral, intracerebroventricular, intradermal, intralesional, intramuscular, intraocular, intraosseous infusion, intraperitoneal, intrapulmonary, intrathecal, intrauterine, intravaginal, intravenous, intravesical, intravitreal, nasal, perivascular, subcutaneous, sublingual, transdermal, topical, transepithelial, or transmucosal. Parenteral administration may be by continuous infusion over a selected period of time.

The compositions and methods of the disclosure may be administered to subjects by a variety of mucosal administration modes, including by oral, rectal, vaginal, intranasal, intrapulmonary, or transdermal or dermal delivery, or by topical delivery to the eyes, ears, skin, or other mucosal surfaces. In some aspects of this disclosure, the mucosal tissue layer includes an epithelial cell layer. The epithelial cell can be pulmonary, tracheal, bronchial, alveolar, nasal, buccal, epidermal, or gastrointestinal. Compositions of this disclosure can be administered using conventional actuators such as mechanical spray devices, as well as pressurized, electrically activated, or other types of actuators.

Compositions of this disclosure may be administered in an aqueous solution as a nasal or pulmonary spray and may be dispensed in spray form by a variety of methods known to those skilled in the art. Pulmonary delivery of a composition of this disclosure is achieved by administering the composition in the form of drops, particles, or spray, which can be, for example, aerosolized, atomized, or nebulized. Particles of the composition, spray, or aerosol can be in either a liquid or solid form. Non-limiting examples of systems for dispensing liquids as a nasal spray are disclosed in U.S. Pat. No. 4,511,069. Such formulations may be conveniently prepared by dissolving compositions according to the present disclosure in water to produce an aqueous solution, and rendering said solution sterile. The formulations may be presented in multi-dose containers, for example in the sealed dispensing system disclosed in U.S. Pat. No. 4,511,069. Other suitable nasal spray delivery systems have been described in TRANSDERMAL SYSTEMIC MEDICATION, Y. W. Chien ed., Elsevier Publishers, New York, 1985; and in U.S. Pat. No. 4,778,810. Additional aerosol delivery forms may include, e.g., compressed air-Jet-, ultrasonic-, and piezoelectric nebulizers, which deliver the biologically active agent dissolved or suspended in a pharmaceutical solvent, e.g., water, ethanol, or mixtures thereof.

Nasal and pulmonary spray solutions of the present disclosure typically comprise the drug or drug to be delivered, optionally formulated with a surface active agent, such as a nonionic surfactant (e.g., polysorbate-80), and one or more buffers. In some embodiments of the present disclosure, the nasal spray solution further comprises a propellant. The pH of the nasal spray solution may be from pH 6.8 to 7.2. The pharmaceutical solvents employed can also be a slightly acidic aqueous buffer of pH 4-6. Other components may be added to enhance or maintain chemical stability, including preservatives, surfactants, dispersants, or gases.

In some embodiments, this disclosure is a pharmaceutical product which includes a solution containing a composition of this disclosure and an actuator for a pulmonary, mucosal, or intranasal spray or aerosol.

A dosage form of the composition of this disclosure can be liquid, in the form of droplets or an emulsion, or in the form of an aerosol.

A dosage form of the composition of this disclosure can be solid, which can be reconstituted in a liquid prior to administration. The solid can be administered as a powder. The solid can be in the form of a capsule, tablet, or gel.

To prepare compositions for pulmonary delivery within the present disclosure, the biologically active agent can be combined with various pharmaceutically acceptable additives, as well as a base or carrier for dispersion of the active agent(s).

Examples of additives include pH control agents such as arginine, sodium hydroxide, glycine, hydrochloric acid, citric acid, and mixtures thereof. Other additives include local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione). When the composition for mucosal delivery is a liquid, the tonicity of the composition, as measured with reference to the tonicity of 0.9% (w/v) physiological saline solution taken as unity, is typically adjusted to a value at which no substantial, irreversible tissue damage will be induced in the mucosa at the site of administration. Generally, the tonicity of the solution is adjusted to a value of ⅓ to 3, more typically ½ to 2, and most often ¾ to 1.7.

The biologically active agent may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g., maleic anhydride) with other monomers (e.g., methyl(meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-gly colic acid) copolymer, polyhydroxybutyric acid, poly(hydroxybutyric acid-gly colic acid) copolymer, and mixtures thereof. Alternatively or additionally, synthetic fatty acid esters such as polyglycerin fatty acid esters, sucrose fatty acid esters, etc., can be employed as carriers. Hydrophilic polymers and other carriers can be used alone or in combination, and enhanced structural integrity can be imparted to the carrier by partial crystallization, ionic bonding, crosslinking, and the like. The carrier can be provided in a variety of forms, including fluid or viscous solutions, gels, pastes, powders, microspheres, and films for direct application to the nasal mucosa. The use of a selected carrier in this context may result in promotion of absorption of the biologically active agent.

Compositions for mucosal, nasal, or pulmonary delivery may contain a hydrophilic low molecular weight compound as a base or excipient. Such hydrophilic low molecular weight compounds may provide a passage medium through which a water-soluble active agent, such as a physiologically active peptide or protein, may diffuse through the base to the body surface where the active agent is absorbed. The hydrophilic low molecular weight compound may optionally absorb moisture from the mucosa or the administration atmosphere and may dissolve the water-soluble active peptide. In some embodiments, the molecular weight of the hydrophilic low molecular weight compound is less than or equal to 10,000, such as not more than 3,000. Examples of hydrophilic low molecular weight compounds include polyol compounds, such as oligo-, di- and monosaccharides including sucrose, mannitol, lactose, L-arabinose, D-erythrose, D-ribose, D-xylose, D-mannose, D-galactose, lactulose, cellobiose, gentibiose, glycerin, polyethylene glycol, and mixtures thereof. Further examples of hydrophilic low molecular weight compounds include N-methylpyrrolidone, alcohols (e.g., oligovinyl alcohol, ethanol, ethylene glycol, propylene glycol, etc.), and mixtures thereof.

The compositions of this disclosure may alternatively contain as pharmaceutically acceptable carriers substances as required to approximate physiological conditions, such as pH adjusting and buffering agents, tonicity adjusting agents, and wetting agents, for example, sodium acetate, sodium lactate, sodium chloride, potassium chloride, calcium chloride, sorbitan monolaurate, triethanolamine oleate, and mixtures thereof. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the disclosure, the biologically active agent may be administered in a time release formulation, for example in a composition which includes a slow release polymer. The active agent can be prepared with carriers that will protect against rapid release, for example a controlled release vehicle such as a polymer, microencapsulated delivery system, or bioadhesive gel. Prolonged delivery of the active agent, in various compositions of the disclosure can be brought about by including in the composition agents that delay absorption, for example, aluminum monosterate hydrogels and gelatin.

In some embodiments, the lipid composition, pharmaceutical compositions, or dosage units contain about 0.01 to about 1000 mg of one or more lipid compounds described herein. In some embodiments, the lipid composition, pharmaceutical compositions, or dosage units contain about 0.01, about 0.1, about 0.5, about 1, about 5, about 10, about 25, about 50, about 75, about 100, about 125, about 150, about 175, about 200, about 225, 250, about 275, about 300, about 350, about 400, about 450, about 500, about 550, about 600, about 650, about 700, about 750, about 800, about 850, about 900, about 950, or about 1000 mg of one or more lipid compounds described herein. In some embodiments, the lipid composition, pharmaceutical compositions, or dosage units contain about 0.01 to about 750 mg, about 0.01 to about 500 mg, about 0.01 to about 250 mg, about 0.01 to about 100 mg, about 0.01 to about 50 mg, about 0.01 to about 25 mg, about 0.01 to about 10 mg, about 0.01 to about 5 mg, about 0.01 to about 0.1 mg, about 0.1 to about 1000 mg, about 0.1 to about 750 mg, about 0.1 to about 500 mg, about 0.1 to about 250 mg, about 0.1 to about 100 mg, about 0.1 to about 50 mg, about 0.1 to about 25, about 0.1 to about 10 mg, about 0.1 to about 5 mg, about 0.1 to about 1 mg, about 1 to about 1000 mg, about 1 to about 750 mg, about 1 to about 500 mg, about 1 to about 250 mg, about 1 to about 100 mg, about 1 to about 50 mg, about 1 to about 25 mg, about 1 to about 10 mg, about 1 to about 5 mg, about 5 to about 1000 mg, about 5 to about 750 mg, about 5 to about 500 mg, about 5 to about 250 mg, about 5 to about 100 mg, about 5 to about 50 mg, about 5 to about 25 mg, about 5 to about 10 mg, about 10 to about 1000 mg, about 10 to about 750 mg, about 10 to about 500, about 10 to about 250 mg, about 10 to about 100 mg, about 10 to about 50 mg, about 10 to about 25 mg, about 25 to about 1000 mg, about 25 to about 750 mg, about 25 to about 500 mg, about 25 to about 250 mg, about 25 to about 100 mg, about 25 to about 50 mg, about 50 to about 1000, mg about 50 to about 750 mg, about 50 to about 500 mg, about 50 to about 250 mg, about 50 to about 100 mg, about 100 to about 1000 mg, about 100 to about 750 mg, about 100 to about 500 mg, about 100 to about 250 mg, about 250 to about 1000 mg, about 250 to about 750 mg, about 250 to about 500 mg, about 500 to about 1000 mg, about 500 to about 750 mg, or about 750 to about 1000 mg of one or more lipid compounds described herein.

### Methods of Using the Lipid Composition

Another aspect of the present disclosure provides methods for delivering a therapeutic agent to a subject (e.g., a patient) in need thereof, comprising administering to said subject (e.g., patient) the pharmaceutical composition comprises a lipid nanoparticle composition comprising a lipid compound of Formula (I), (IA-1), (IA-2), (IIA)-(IIC), (IIA-1), (IIIA)-(IIIE), (IVA-1)-(IVA-3), (IVC-1)-(IVC-2), and (VC-1)-(VC-6), a pharmaceutically acceptable salt thereof, and/or a stereoisomer of any of the foregoing, and the therapeutic agent.

Another aspect of the present disclosure relates to a method of extrahepatic delivery of a therapeutic agent to at least one organ other than liver (e.g., the pancreas, one or both lungs, or the spleen) of a subject in need thereof with a minimum amount delivered elsewhere in body, such as in the liver, of the subject. The method comprises administering to said subject the pharmaceutical composition comprises a lipid nanoparticle composition comprising a lipid compound of Formula (I), (IA-1), (IA-2), (IIA)-(IIC), (IIA-1), (IIIA)-(IIIE), (IVA-1)-(IVA-3), (IVC-1)-(IVC-2), and (VC-1)-(VC-6), a pharmaceutically acceptable salt thereof, and/or a stereoisomer of any of the foregoing, and the therapeutic agent. In some embodiments, the method delivers the therapeutic agent to the pancreas and/or one or both lungs a subject in need thereof with a minimum amount delivered elsewhere in body, such as in the liver, of the subject.

In some embodiments, less than 55%, 50%, 45%, 40%, 35%, 30%, 25%, 20%, 15%, 10%, 5% or 1% of the total therapeutic agent administered to the subject is delivered to the liver of the subject. In some embodiments, less than 6%, 7%, 8%, 10%, 11%, 12%, 13%, 14%, 15%, 16%, 17%, 18%, 19%, or 20% of the total therapeutic agent administered to the subject is delivered to the liver of the subject.

In some embodiments, more than 99%, 95%, 90%, 85%, 80%, 75%, 70%, 65%, 60%, 55%, 50%, 45%, 40%, 35%, 30%, 25%, 20%, 15%, or 10% of the total therapeutic agent administered to the subject is delivered to the pancreas, spleen, and/or one or both lungs of the subject. In some embodiments, more than 99%, 95%, 90%, 85%, 80%, 75%, 70%, 65%, 60%, 55%, 50%, 45%, 40%, 35%, 30%, 25%, 20%, 15%, or 10% of the total therapeutic agent administered to the subject is delivered to the pancreas of the subject. In some embodiments, more than 99%, 95%, 90%, 85%, 80%, 75%, 70%, 65%, 60%, 55%, 50%, 45%, 40%, 35%, 30%, 25%, 20%, 15%, or 10% of the total therapeutic agent administered to the subject is delivered to the lungs of the subject. In some embodiments, more than 99%, 95%, 90%, 85%, 80%, 75%, 70%, 65%, 60%, 55%, 50%, 45%, 40%, 35%, 30%, 25%, 20%, 15%, or 10% of the total therapeutic agent administered to the subject is delivered to the spleen of the subject.

In some embodiments, the total therapeutic agent administered to the subject has a spleen to liver ratio of at least 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, or 10. In some embodiments, the total therapeutic agent administered to the subject has a spleen to liver ratio of at least 15, 20, 25, 30, 35, 40, 45, 50, 55, 60, 65, 70, 75, 80, 85, 90, 95, 100, 105, 110, 115, 120, 125, 130, 135, 140, 145, or 150. In some embodiments, the total therapeutic agent administered to the subject has a spleen to liver ratio of at least 1. In some embodiments, the total therapeutic cargo administered to the subject has spleen to liver ratio of at least 5. In some embodiments, the total therapeutic agent administered to the subject has a spleen to liver ratio of at least 10. In some embodiments, the total therapeutic agent administered to the subject has a spleen to liver ratio of at least 25. In some embodiments, the total therapeutic agent administered to the subject has a spleen to liver ratio of at least 70. In some embodiments, the total therapeutic agent administered to the subject has a spleen to liver ratio of at least 75. In some embodiments, the total therapeutic agent administered to the subject has a spleen to liver ratio of at least 100. In some embodiments, the total therapeutic agent administered to the subject has a spleen to liver ratio of at least 110.

As used herein, the percent amount of the total therapeutic agent administered to the subject and delivered to a location in the subject is measured by the level of protein expression, or mRNA knockdown level.

In some embodiments, the method of delivering a therapeutic agent disclosed above comprises administering to a subject a lipid composition comprising therapeutic agent. In some embodiments, the lipid nanoparticles in the lipid composition are formed from one or more compounds chosen from ionizable lipids of Formula (I), (IA-1), (IA-2), (IIA)-(IIC), (IIA-1), (IIIA)-(IIIIE), (IVA-1)-(IVA-3), (IVC-1)-(IVC-2), and (VC-1)-(VC-6), pharmaceutically acceptable salts thereof, and stereoisomers of any of the foregoing. In some embodiments, the lipid nanoparticles are formed from one or more compounds chosen from ionizable lipids of Formula (I), (IA-1), or (IA-2), pharmaceutically acceptable salts thereof, and stereoisomers of any of the foregoing. In some embodiments, the lipid nanoparticles are formed from one or more compounds chosen from ionizable lipids of Formula (IIA)-(IIC), pharmaceutically acceptable salts thereof, and stereoisomers of any of the foregoing. In some embodiments, the lipid nanoparticles are formed from one or more compounds chosen from ionizable lipids of Formula (IIA-1), pharmaceutically acceptable salts thereof, and stereoisomers of any of the foregoing. In some embodiments, the lipid nanoparticles are formed from one or more compounds chosen from ionizable lipids of Formula (IIIA)-(IIIIE), pharmaceutically acceptable salts thereof, and stereoisomers of any of the foregoing. In some embodiments, the lipid nanoparticles are formed from one or more compounds chosen from ionizable lipids of Formula (IVA-1)-(IVA-3), pharmaceutically acceptable salts thereof, and stereoisomers of any of the foregoing. In some embodiments, the lipid nanoparticles are formed from one or more compounds chosen from ionizable lipids of Formula (IVC-1)-(IVC-3), pharmaceutically acceptable salts thereof, and stereoisomers of any of the foregoing. In some embodiments, the lipid nanoparticles are formed from one or more compounds chosen from ionizable lipids of Formula (VC-1)-(VC-6), pharmaceutically acceptable salts thereof, and stereoisomers of any of the foregoing.

In some embodiments, the lipid compositions disclosed herein may be used for a variety of purposes, including delivery of encapsulated or associated (e.g., complexed) therapeutic agents such as nucleic acids to cells, in vitro and/or in vivo. Accordingly, in some embodiments, provided are methods of treating or preventing diseases or disorders in a subject in need thereof comprising administering to the subject a lipid composition. In some embodiments, the lipid composition encapsulates or is associated with a suitable therapeutic agent, wherein the lipid composition comprises one or more of the novel ionizable lipids described herein, a pharmaceutically acceptable salt thereof, and/or a stereoisomer of any of the foregoing.

In some embodiments, the lipid compositions of the present disclosure are useful for delivery of therapeutic agent. In some embodiments, the therapeutic agent is chosen from one or more nucleic acids, including, e.g., mRNA, antisense oligonucleotide, plasmid DNA, microRNA (miRNA), miRNA inhibitors (antagomirs/antimirs), messenger-RNA-interfering complementary RNA (micRNA), DNA, multivalent RNA, dicer substrate RNA, complementary DNA (cDNA), etc. Therefore, in some embodiments, disclosed herein are methods of inducing expression of a desired protein in vitro and/or in vivo by contacting cells with a lipid composition comprising one or more novel ionizable lipids described herein, wherein the lipid nanoparticle encapsulates or is associated with a nucleic acid that is expressed to produce a desired protein (e.g., a messenger RNA or plasmid encoding the desired protein) or inhibit processes that terminate expression of m RNA (e.g., miRNA inhibitors). In some embodiments, disclosed herein are methods of decreasing expression of target genes and proteins in vitro and/or in vivo by contacting cells with a lipid composition comprising one or more novel ionizable lipids described herein, wherein the lipid nanoparticle encapsulates or is associated with a nucleic acid that reduces target gene expression (e.g., an antisense oligonucleotide or small interfering RNA (siRNA)). In some embodiments, disclosed herein are methods for co-delivery of one or more nucleic acid (e.g. mRNA and plasmid DNA), separately or in combination, such as may be useful to provide an effect requiring colocalization of different nucleic acids (e.g. mRNA encoding for a suitable gene modifying enzyme and DNA segment(s) for incorporation into the host genome).

In some embodiments, the lipid compositions are useful for upregulation of endogenous protein expression by delivering mRNA inhibitors targeting one specific miRNA or a group of miRNA regulating one target mRNA or several mRNA. In some embodiments, provided herein are methods for upregulating endogenous protein expression comprising delivering miRNA inhibitors targeting one or more miRNA regulating one or more mRNA. In some embodiments, the lipid compositions are useful for down-regulating (e.g., silencing) the protein levels and/or mRNA levels of target genes. In some embodiments, provided herein are methods for down-regulating (e.g., silencing) protein and/or mRNA levels of target genes.

In some embodiments, the lipid composition are useful for delivery of mRNA and plasmids for expression of transgenes. In some embodiments, provided herein are methods for delivering mRNA and plasmids for expression of transgenes.

In some embodiments, the lipid compositions are useful for inducing a pharmacological effect resulting from expression of a protein, e.g., increased production of red blood cells through the delivery of a suitable erythropoietin mRNA, or protection against infection through delivery of mRNA encoding for a suitable antigen or antibody. In some embodiments, provided herein are methods for inducing a pharmacological effect resulting from expression of a protein, e.g., increased production of red blood cells through the delivery of a suitable erythropoietin mRNA, or protection against infection through delivery of mRNA encoding for a suitable antigen or antibody.

Non-limiting exemplary embodiments of the ionizable lipids of the present disclosure, lipid compositions comprising the same, and their use to deliver agents (e.g., therapeutic agents, such as nucleic acids) and/or to modulate gene and/or protein expression are described in further detail below.

In some embodiments, the disclosure relates to a method of gene editing, comprising contacting a cell with the LNP composition. In some embodiments, the disclosure relates to any method of gene editing described herein, comprising cleaving DNA.

In some embodiments, the disclosure relates to a method of cleaving DNA, comprising contacting a cell with an LNP composition.

In some embodiments, the disclosure relates to any method of cleaving DNA described herein, wherein the cleaving step comprises introducing a single stranded DNA nick. In some embodiments, the disclosure relates to any method of cleaving DNA described herein, wherein the cleaving step comprises introducing a double-stranded DNA break. In some embodiments, the disclosure relates to any method of cleaving DNA described herein, wherein the LNP composition comprises a Class 2 Cas mRNA and a guide RNA nucleic acid.

In some embodiments, the disclosure relates to any method of cleaving DNA described herein, further comprising introducing at least one template nucleic acid into the cell. In some embodiments, the disclosure relates to any method of cleaving DNA described herein, comprising contacting the cell with an LNP composition comprising a template nucleic acid.

In some embodiments, the disclosure relates to any a method of gene editing described herein, wherein the method comprises administering the LNP composition to an animal, for example a human. In some embodiments, the disclosure relates to any method of gene editing described herein, wherein the method comprises administering the LNP composition to a cell, such as a eukaryotic cell.

In some embodiments, the disclosure relates to any method of gene editing described herein, wherein the method comprises administering the mRNA formulated in a first LNP composition and a second LNP composition comprising one or more of an mRNA, a gRNA, a gRNA nucleic acid, and a template nucleic acid. In some embodiments, the disclosure relates to any method of gene editing described herein, wherein the first and second LNP compositions are administered simultaneously. In some embodiments, the disclosure relates to any method of gene editing described herein, wherein the first and second LNP compositions are administered sequentially.

In some embodiments, the disclosure relates to any method of gene editing described herein, wherein the method comprises administering the mRNA and the guide RNA nucleic acid formulated in a single LNP composition. In some embodiments, the disclosure relates to any method of gene editing described herein, wherein the gene editing results in a gene knockout. In some embodiments, the disclosure relates to any method of gene editing described herein, wherein the gene editing results in a gene correction.

In some embodiments, the disclosure relates to methods for in vivo delivery of interfering RNA to the lung of a mammalian subject.

In some embodiments, relates to methods of treating a disease or disorder in a mammalian subject. In some embodiments, these methods comprise administering a therapeutically effective amount of the lipid composition of this disclosure to a subject having a disease or disorder associated with expression or overexpression of a gene that can be reduced, decreased, downregulated, or silenced by the lipid composition.

### EXAMPLES

The following examples are for illustrative purposes only and are not intended to limit, in any way, the scope of the present invention.

### Example 1. Synthesis of Compound 2230

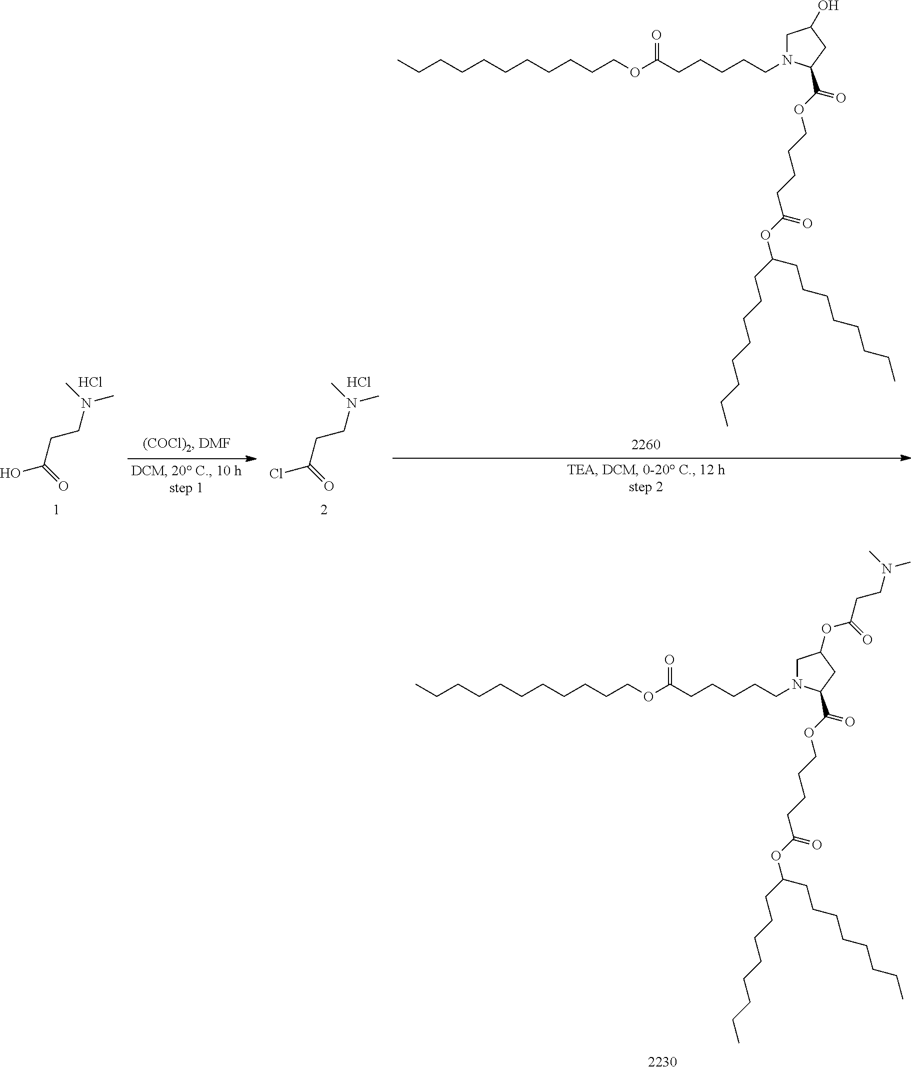

### Step 1

To a solution of 3-(dimethylamino)propanoic acid (0.2 g, 1.30 mmol, 1 eq., HCl) and oxalyl dichloride (826.30 mg, 6.51 mmol, 569.86 μL, 5 eq.) in DCM (5 mL), was added two drops of DMF (9.52 mg, 130.20 μmol, 10.02 μL, 0.1 eq.). The mixture was degassed and purged with N₂ for 3 times, and stirred at 20° C. for 10 hours under N₂ atmosphere. The reaction mixture was concentrated under reduced pressure to give 3-(dimethylamino)propanoyl chloride (0.2 g, crude, HCl) as yellow oil.

### Step 2

To a solution of [5-(1-octylnonoxy)-S-oxo-pentyl](2S)-4-hydroxy-1-(6-oxo-6-undecoxy-hexyl) pyrrolidine-2-carboxylate (100.00 mg, 135.48 μmol, 1 eq.), 3-(dimethylamino)propanoyl chloride (93.24 mg, 541.91 μmol, 4 eq., HCl) in DCM (3 mL), was added TEA (123.38 mg, 1.22 mmol, 169.71 μL, 9 eq.) at 0° C. The mixture was stirred at 20° C. for 12 hours. The reaction mixture was diluted with H₂O 20 mL and extracted with 60 mL EtOAc (20 mL×3). The combined organic layers were dried over Na₂SO₄, filtered, and concentrated under reduced pressure to give a residue. The residue was purified by column chromatography (SiO₂, petroleum ether/ethyl acetate/NH₃·H₂O=1/0/0.1 to 3/1/0.1) and preparative HPLC (column: phenomenex Luna C18 100×30 mm×5 μm; mobile phase: [water(HCl)-ACN]; B %: 45%-75%, 10 minutes) to give a residue. The residue was adjusted to pH=7 with saturated aqueous NaHCO₃ and extracted with 60 mL EtOAc (20 mL×3). The combined organic layers were dried over Na₂SO₄, filtered, and concentrated under reduced pressure to give a residue. Then the residue was purified by column chromatography (SiO₂, petroleum ether/ethyl acetate/NH₃·H₂O=1/0/0.1 to 5/1/10.1) to give 2230, [5-(1-octylnonoxy)-S-oxo-pentyl](2S)-4-[3-(dimethylamino)propanoyloxy]-1-(6-oxo-6-undecoxy-hexyl)pyrrolidine-2-carboxylate (17 mg, 20.30 μmol, 14.99% yield, 100% purity) as yellow oil.

¹H NMR (400 MHz, CDCl₃), 5.21-5.27 (m, 1H), 4.86-4.89 (m, 1H), 4.14-4.16 (m, 2H), 4.06 (t, J=6.4 Hz, 2H), 3.44-3.55 (m, 1H), 3.10-3.26 (m, 1H), 2.45-2.73 (m, 7H), 2.32-2.33 (m, 10H), 2.16-2.28 (m, 1H), 2.05-2.06 (m, 1H), 1.60-1.65 (m, 6H), 1.51-1.52 (m, 6H), 1.27-1.31 (m, 46H), 0.89 (t, J=6.8 Hz, 9H). LCMS: (1/2M+H⁺): 419.2 @2.971 minutes.

### Example 2. Synthesis of Compound 2260

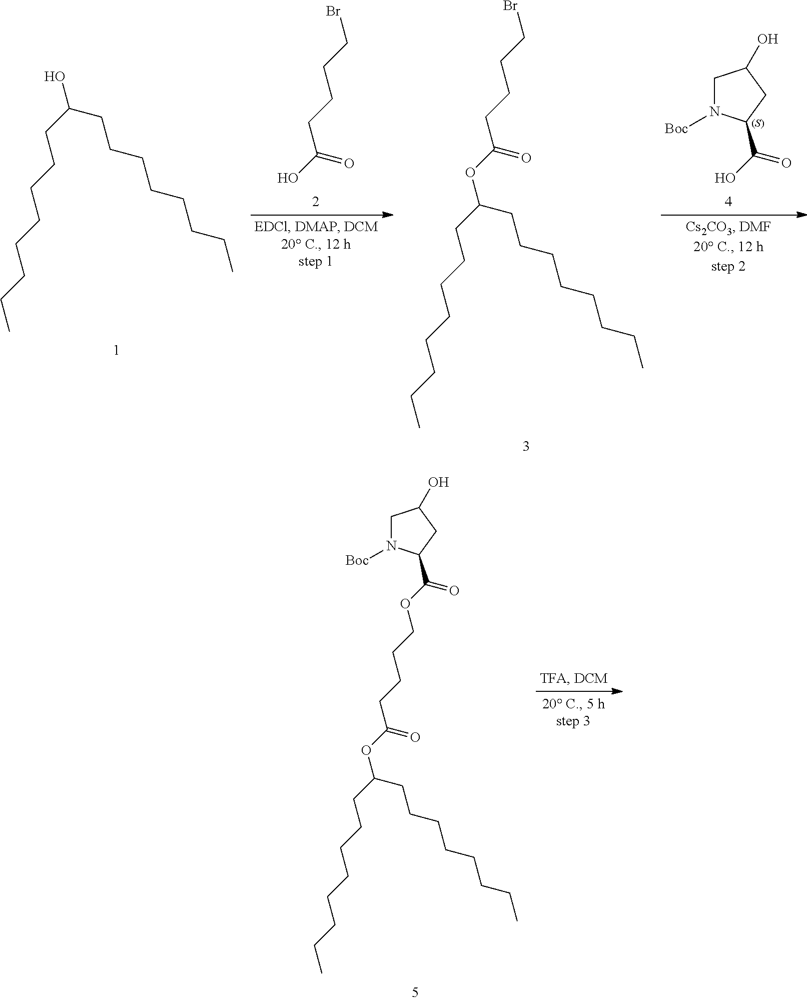

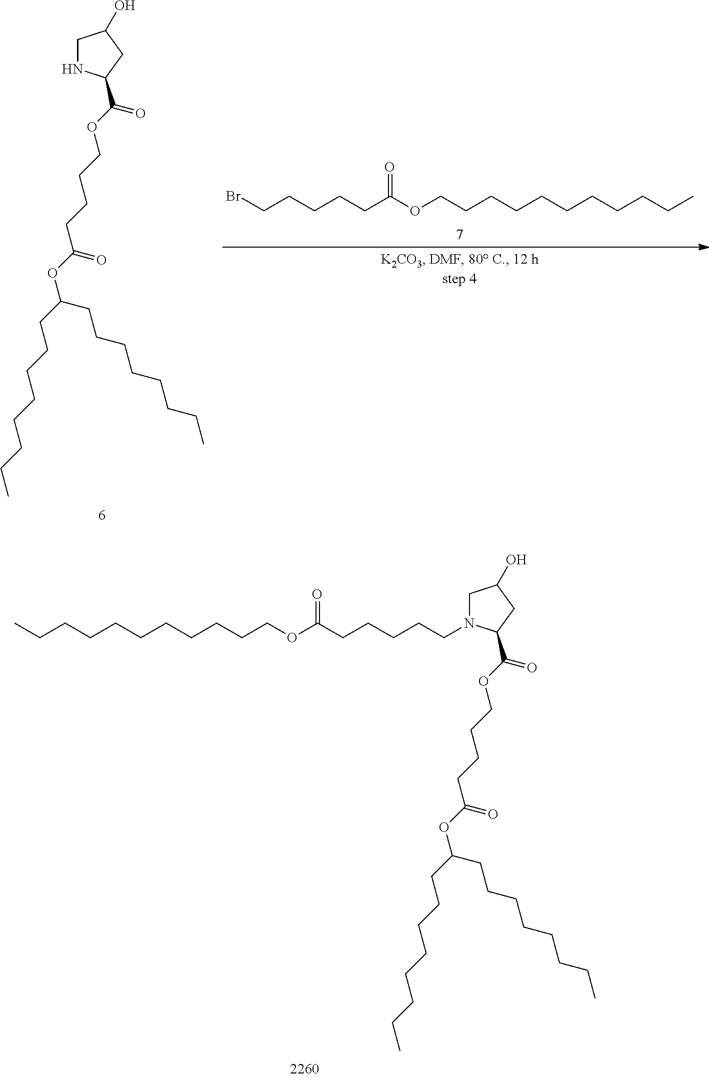

### Step 1

A solution of heptadecan-9-ol (10 g, 38.99 mmol, 1 eq.), 5-bromopentanoic acid (7.06 g, 38.99 mmol, 1 eq.), DMAP (952.72 mg, 7.80 mmol, 0.2 eq.), and EDCI (7.47 g, 38.99 mmol, 1 eq.) in DCM (70 mL) was stirred at 20° C. for 12 hours. The combined organic phase was diluted with 200 mL EtOAc and washed with 600 mL water (200 mL×3) and 400 mL brine (200 mL×2), dried with anhydrous Na₂SO₄, filtered, and concentrated under reduced pressure to give a residue. The residue was purified by column chromatography (SiO₂, petroleum ether/ethyl acetate=20/1 to 10/1) to give 1-octylnonyl 5-bromopentanoate (25 g, 59.60 mmol, 76.42% yield) as colorless oil.

1H NMR (400 MHz, CDCl₃), 4.85-4.91 (m, 1H), 3.42 (t, J=6.8 Hz, 2H), 2.34 (t, J=7.2 Hz, 2H), 1.90-1.93 (m, 2H), 1.79-1.81 (m, 2H), 1.52-1.57 (m, 4H), 1.27-1.51 (m, 24H), 0.89 (t, J=6.4 Hz, 6H).

### Step 2

A mixture of (2S)-1-tert-butoxycarbonyl-4-hydroxy-pyrrolidine-2-carboxylic acid (2.30 g, 9.93 mmol, 1 eq.), 1-octylnonyl 5-bromopentanoate (5 g, 11.92 mmol, 1.2 eq.), Cs₂CO₃ (7.12 g, 21.85 mmol, 2.2 eq.) in DMF (60 mL) was stirred at 20° C. for 12 hours under N₂ atmosphere. The reaction mixture was quenched by adding 10 mL H₂O at 0° C. The mixture was extracted with 30 mL EtOAc (10 mL×3) and the combined organic layers were dried over Na₂SO₄, filtered, and concentrated under reduced pressure to give a residue. The residue was purified by column chromatography (SiO₂, petroleum ether/ethyl acetate=10/1 to 3/1) to give O1-tert-butyl O2-[5-(1-octylnonoxy)-S-oxo-pentyl](2S)-4-hydroxypyrrolidine-1,2-dicarboxylate (4.5 g, 3.95 mmol, 39.75% yield, 50% purity) as a white solid.

¹H NMR (400 MHz, CDCl₃), 4.86-4.89 (m, 1H), 4.17-4.39 (m, 4H), 3.50-3.69 (m, 2H), 2.33-2.34 (m, 2H), 1.61-1.72 (m, 4H), 1.42-1.52 (m, 14H), 1.25-1.30 (m, 25H), 0.88 (t, J=6.8 Hz, 6H).

### Step 3

To a solution of O1-tert-butyl O2-[5-(1-octylnonoxy)-S-oxo-pentyl](2S)-4-hydroxypyrrolidine-1,2-dicarboxylate (4.00 g, 7.02 mmol, 1 eq.) in DCM (50 mL), was added TFA (23.10 g, 202.60 mmol, 15.00 mL, 28.86 eq.). The mixture was stirred at 20° C. for 5 hours. The reaction mixture was adjusted to pH=7 with saturated aqueous NaHCO₃ and extracted with 600 mL EtOAc (200 mL×3), dried over Na₂SO₄, filtered, and concentrated under reduced pressure to give a residue. The residue was purified by column chromatography (SiO₂, petroleum ether/ethyl acetate=0/1 to ethyl acetate/MeOH=3/1) to give [5-(1-octylnonoxy)-S-oxo-pentyl](2S)-4-hydroxypyrrolidine-2-carboxylate (2.5 g, 4.52 mmol, 64.45% yield, 85% purity) as yellow oil.

¹H NMR (400 MHz, CDCl₃), 4.86-4.89 (m, 1H), 4.37-4.45 (m, 1H), 4.15-4.25 (m, 2H), 3.82-4.14 (m, 1H), 2.97-3.15 (m, 2H), 2.33-2.35 (m, 2H), 1.69-1.70 (m, 4H), 1.50-1.52 (m, 4H), 1.26-1.32 (m, 26H), 0.88 (t, J=6.8 Hz, 6H).

### Step 4

To a solution of [5-(1-octylnonoxy)-S-oxo-pentyl](2S)-4-hydroxypyrrolidine-2-carboxylate (500.00 mg, 1.06 mmol, 1 eq.), undecyl 6-bromohexanoate (446.26 mg, 1.28 mmol, 1.2 eq.) in DMF (10 mL) was added K₂CO₃ (441.37 mg, 3.19 mmol, 3 eq.). The mixture was stirred at 80° C. for 12 hours. The reaction mixture was diluted with 20 mL H₂O and extracted with 60 mL EtOAc (20 mL×3). The combined organic layers were dried over Na₂SO₄, filtered, and concentrated under reduced pressure to give a residue. The residue was purified by column chromatography (SiO₂, petroleum ether/ethyl acetate/NH₃·H₂O=10/1/1 to 1/1/0.5) to give 2260, [5-(1-octylnonoxy)-S-oxo-pentyl](2S)-4-hydroxy-1-(6-oxo-6-undecoxy-hexyl)pyrrolidine-2-carboxylate (0.6 g, 715.32 μmol, 67.20% yield, 88% purity) as yellow oil.

¹H NMR (400 MHz, CDCl₃), 4.86-4.91 (m, 1H), 4.28-4.49 (m, 1H), 4.13-4.16 (m, 2H), 4.06 (t, J=6.4 Hz, 2H), 3.07-3.54 (m, 2H), 2.48-2.65 (m, 3H), 2.28-2.34 (m, 4H), 1.95-2.23 (m, 2H), 1.60-1.64 (m, 6H), 1.50-1.52 (m, 6H), 1.27-1.35 (m, 44H), 0.89 (t, J=6.4 Hz, 9H), (M+H⁺): 738.3. LCMS: (M+H⁺): 738.3 @2.843 minutes.

### Example 3. Synthesis of Compound 2231

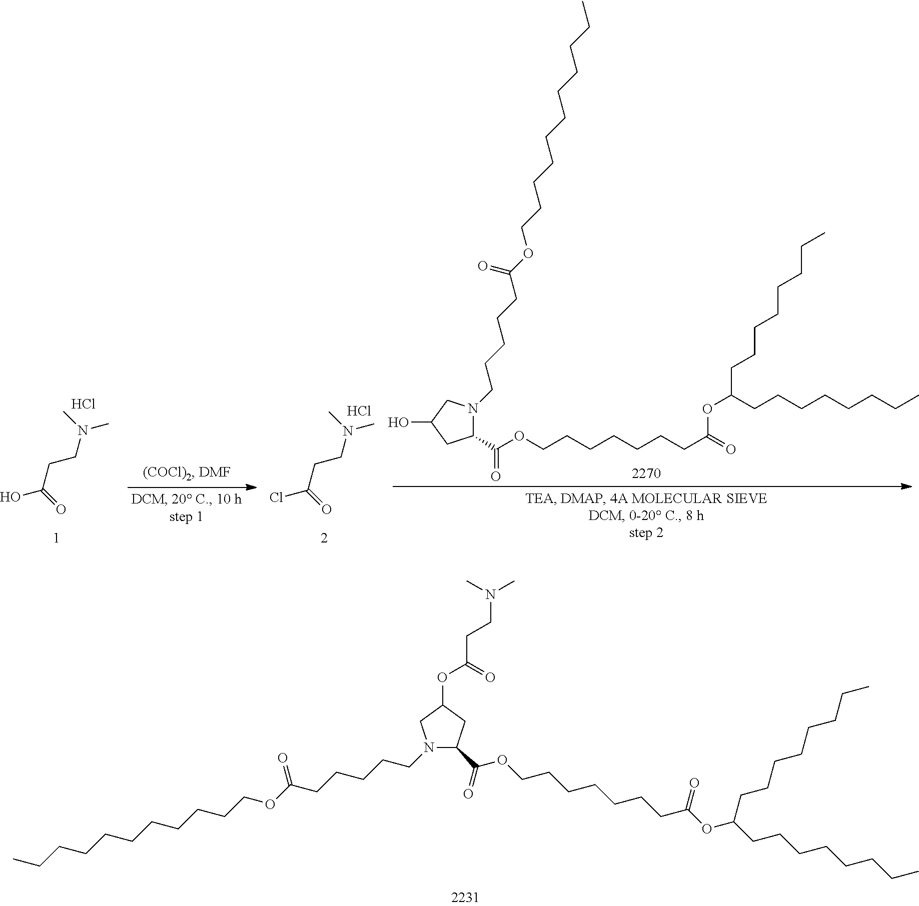

### Step 1

To a solution of 3-(dimethylamino)propanoic acid (0.2 g, 1.30 mmol, 1 eq., HCl) and oxalyl dichloride (826.30 mg, 6.51 mmol, 569.86 μL, 5 eq.) in DCM (5 mL), was added two drops of DMF (9.52 mg, 130.20 μmol, 10.02 μL, 0.1 eq.) at 20° C. The mixture was stirred at 20° C. for 10 hours under N₂ atmosphere. The reaction mixture was concentrated under reduced pressure to give 3-(dimethylamino)propanoyl chloride (0.2 g, crude, HCl) as yellow oil.

### Step 2

To a suspension of [8-(1-octylnonoxy)-8-oxo-octyl](2S)-4-hydroxy-1-(6-oxo-6-undecoxy-hexyl) pyrrolidine-2-carboxylate (200 mg, 256.34 μmol, 1 eq.), DMAP (6.26 mg, 51.27 μmol, 0.2 eq), TEA (207.51 mg, 2.05 mmol, 285.44 μL, 8 eq.) and 4A molecular sieve (100 mg) in DCM (15 mL), was added 3-(dimethylamino)propanoyl chloride (220.52 mg, 1.28 mmol, 5 eq., HCl) in DCM (10 mL) at 0° C. The mixture was stirred at 20° C. for 8 hours under N₂ atmosphere. The reaction mixture was diluted with 20 mL H₂O and extracted with 60 mL EtOAc (20 mL×3). The combined organic layers were dried over Na₂SO₄, filtered, and concentrated under reduced pressure to give a residue. The residue was purified by column chromatography (SiO₂, petroleum ether/ethyl acetate/NH₃·H₂O=1/0/0.1 to 3/1/0.1) and preparative HPLC (column: Phenomenex Luna C18 100×30 mm×5 μm; mobile phase: [water(HCl)-ACN]; B %: 55%-85%, 10 minutes) to give a residue. The residue was adjusted to pH=7 with saturated aqueous NaHCO₃ and extracted with 60 mL EtOAc (20 mL×3). The combined organic layers were dried over Na₂SO₄, filtered, and concentrated under reduced pressure to give a residue. Then the residue was purified by column chromatography (SiO₂, petroleum ether/ethyl acetate/NH₃·H₂O=1/0/0.1 to 5/1/0.1) to give 2231, [8-(1-octylnonoxy)-8-oxo-octyl](2S)-4-[3-(dimethylamino)propanoyloxy]-1-(6-oxo-6-undecoxy-hexyl)pyrrolidine-2-carboxylate (40 mg, 45.49 μmol, 13.33% yield, 100% purity) as yellow oil.

1H NMR (400 MHz, CDCl₃), 5.21-5.27 (m, 1H), 4.84-4.90 (m, 1H), 4.04-4.13 (m, 4H), 3.08-3.54 (m, 2H), 2.32-2.65 (m, 7H), 2.29-2.31 (m, 10H), 2.27-2.28 (m, 2H), 1.63-1.65 (m, 8H), 1.60-1.62 (m, 6H), 1.27-1.52 (m, 48H), 0.89 (t, J=6.4 Hz, 9H). LCMS: (1/2M+H⁺): 879.6 @2.155 minutes.

### Example 4. Synthesis of Compound 2270

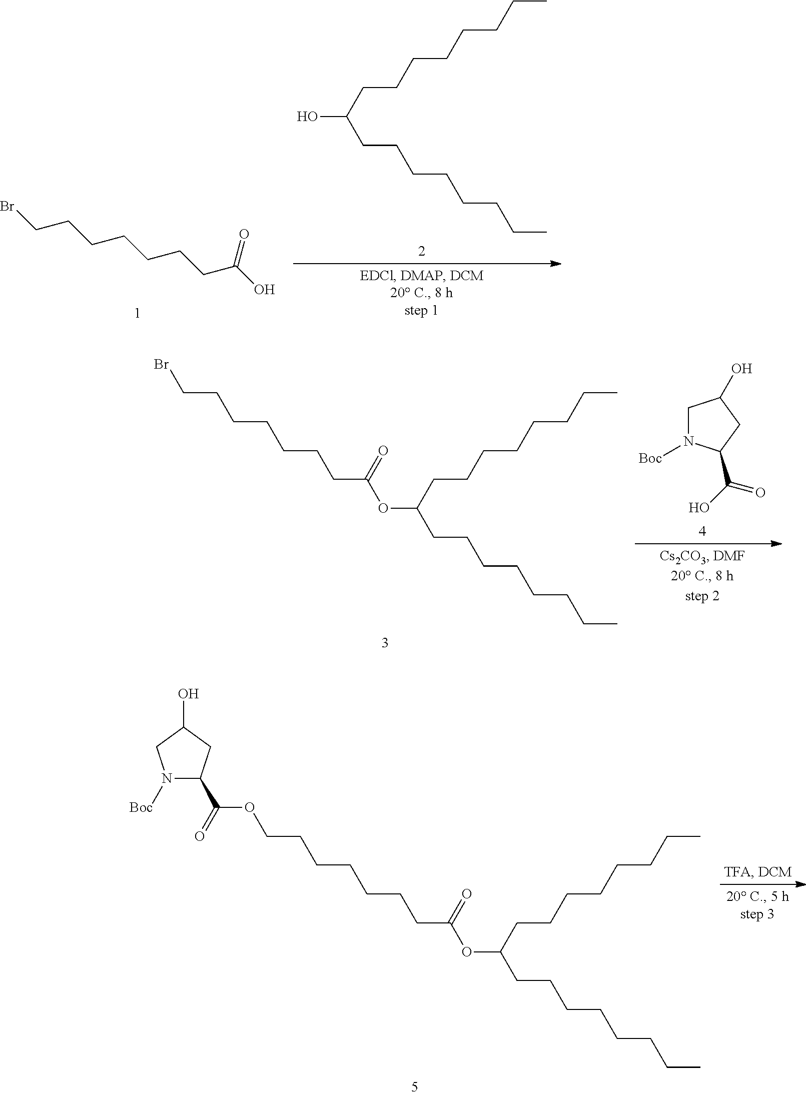 [SMILES: O=C(O)CCCCCCCBr ; CCCCCCCCC(O)CCCCCCCC ; CCCCCCCCC(CCCCCCCC)OC(=O)CCCCCCCBr ; CC(C)(C)OC(=O)N1CC(O)C[C@H]1C(=O)O ; CCCCCCCCC(CCCCCCCC)OC(=O)CCCCCCCOC(=O)[C@@H]1CC(O)CN1C(=O)OC(C)(C)C]

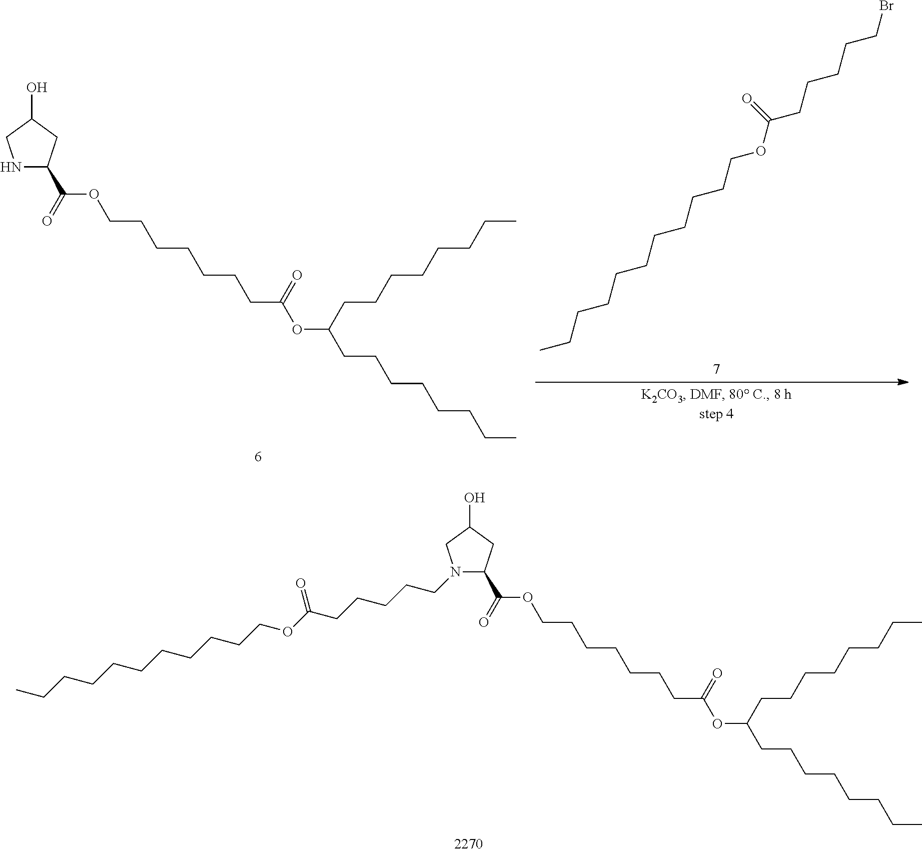 [SMILES: CCCCCCCCC(CCCCCCCC)OC(=O)CCCCCCCOC(=O)[C@@H]1CC(O)CN1 ; CCCCCCCCCCCOC(=O)CCCCCBr ; CCCCCCCCCCCOC(=O)CCCCCN1CC(O)C[C@H]1C(=O)OCCCCCCCC(=O)OC(CCCCCCCC)CCCCCCCC]

### Step 1

A mixture of 8-bromooctanoic acid (10 g, 44.82 mmol, 1 eq.) in DCM (1000 mL) was added DMAP (1.10 g, 8.96 mmol, 0.2 eq), heptadecan-9-ol (11.50 g, 44.82 mmol, 1 eq.), and EDCI (8.59 g, 44.82 mmol, 1 eq.), and was degassed and purged with N₂ for 3 times. The mixture was stirred at 20° C. for 8 hours under N₂ atmosphere. The reaction was diluted with 200 mL EtOAc and washed with 600 mL water (200 mL×3) and 400 mL brine (200 mL×2), dried with anhydrous Na₂SO₄, filtered, and concentrated under reduced pressure to give a residue. The residue was purified by column chromatography (SiO₂, petroleum ether/ethyl acetate=1/0 to 1/0) to give μl-octylnonyl 5-bromopentanoate (25 g, 59.60 mmol, 76.42% yield) as colorless oil.

1H NMR (400 MHz, CDCl₃), 4.86-4.89 (m, 1H), 3.41 (t, J=7.2 Hz, 2H), 2.29 (t, J=7.6 Hz, 2H), 1.80-1.90 (m, 2H), 1.60-1.63 (m, 2H), 1.44-1.51 (m, 4H), 1.34-1.35 (m, 2H), 1.27-1.33 (m, 28H), 0.89 (t, J=6.8 Hz, 6H).

### Step 2

A mixture of (2S)-1-tert-butoxycarbonyl-4-hydroxy-pyrrolidine-2-carboxylic acid (3.34 g, 14.44 mmol, 1 eq.), 1-octylnonyl 8-bromooctanoate (8 g, 17.33 mmol, 1.2 eq.), Cs₂CO₃ (10.35 g, 31.78 mmol, 2.2 eq.) in DMF (60 mL) was stirred at 20° C. for 8 hours under N₂ atmosphere. The reaction mixture was quenched by adding 10 mL H₂O at 0° C. The mixture was extracted with 30 mL EtOAc (10 mL×3) and the combined organic layers were dried over Na₂SO₄, filtered, and concentrated under reduced pressure to give a residue. The residue was purified by column chromatography (SiO₂, petroleum ether/ethyl acetate=10/1 to 3/1) to give O1-tert-butyl O2-[8-(1-octylnonoxy)-8-oxo-octyl](2S)-4-hydroxypyrrolidine-1,2-dicarboxylate (6 g, 9.81 mmol, 67.89% yield) as a white solid.

### Step 3

To a solution of O1-tert-butyl O2-[8-(1-octylnonoxy)-8-oxo-octyl](2S)-4-hydroxypyrrolidine-1,2-dicarboxylate (5.5 g, 8.99 mmol, 1 eq.) in DCM (50 mL), was added TFA (23.10 g, 202.59 mmol, 15 mL, 22.54 eq.). The mixture was stirred at 20° C. for 5 hours. The reaction mixture was adjusted to pH=7 with saturated aqueous NaHCO₃ and extracted with 600 mL EtOAc (200 mL×3), dried over Na₂SO₄, filtered, and concentrated under reduced pressure to give a residue. The residue was purified by column chromatography (SiO₂, petroleum ether/ethyl acetate=0/1 to ethyl acetate/MeOH=3/1) to give [5-(1-octylnonoxy)-S-oxo-pentyl](2S)-4-hydroxypyrrolidine-2-carboxylate (2.5 g, 4.52 mmol, 64.45% yield, 85% purity) as yellow oil.

1H NMR (400 MHz, CDCl₃), 4.85-4.89 (m, 1H), 4.45-4.47 (m, 1H), 4.02-4.18 (m, 3H), 2.99-3.19 (m, 2H), 2.29-2.31 (m, 4H), 2.07-2.27 (m, 1H), 2.05-2.06 (m, 1H), 1.61-1.66 (m, 4H), 1.50-1.52 (m, 4H), 1.26-1.35 (m, 30H), 0.88 (t, J=6.8 Hz, 6H).

### Step 4

To a solution of [8-(1-octylnonoxy)-8-oxo-octyl](2S)-4-hydroxypyrrolidine-2-carboxylate (2 g, 3.91 mmol, 1 eq.), undecyl 6-bromohexanoate (1.64 g, 4.69 mmol, 1.2 eq.) in DMF (20 mL), was added K₂CO₃ (1.62 g, 11.72 mmol, 3 eq.). The mixture was stirred at 80° C. for 8 hours. The reaction mixture was diluted with 20 mL H₂O and extracted with 60 mL EtOAc (20 mL×3). The combined organic layers were dried over Na₂SO₄, filtered, and concentrated under reduced pressure to give a residue. The residue was purified by column chromatography (SiO₂, petroleum ether/ethyl acetate/NH₃·H₂O=10/1/1 to 1/1/0.5) to give 2270 [8-(1-octylnonoxy)-8-oxo-octyl](2S)-4-hydroxy-1-(6-oxo-6-undecoxy-hexyl)pyrrolidine-2-carboxylate (3 g, 3.85 mmol, 98.39% yield, 100% purity) as yellow oil.

¹H NMR (400 MHz, CDCl₃), 4.84-4.90 (m, 1H), 4.49-4.52 (m, 1H), 4.04-4.39 (m, 5H), 3.05-3.66 (m, 2H), 2.48-2.69 (m, 2H), 1.94-2.32 (m, 6H), 1.60-1.66 (m, 8H), 1.50-1.52 (m, 6H), 1.27-1.34 (m, 48H), 0.89 (t, J=6.8 Hz, 9H). LCMS: (M+H⁺): 780.5 @2.889 minutes.

### Example 5. General Reaction Schemes for Synthesis of Exemplary Ionizable Lipid Compounds

General reaction schemes for synthesis of exemplary ionizable lipid compounds, containing heterocyclic core structure (e.g., N-containing core) is shown in Scheme 1.

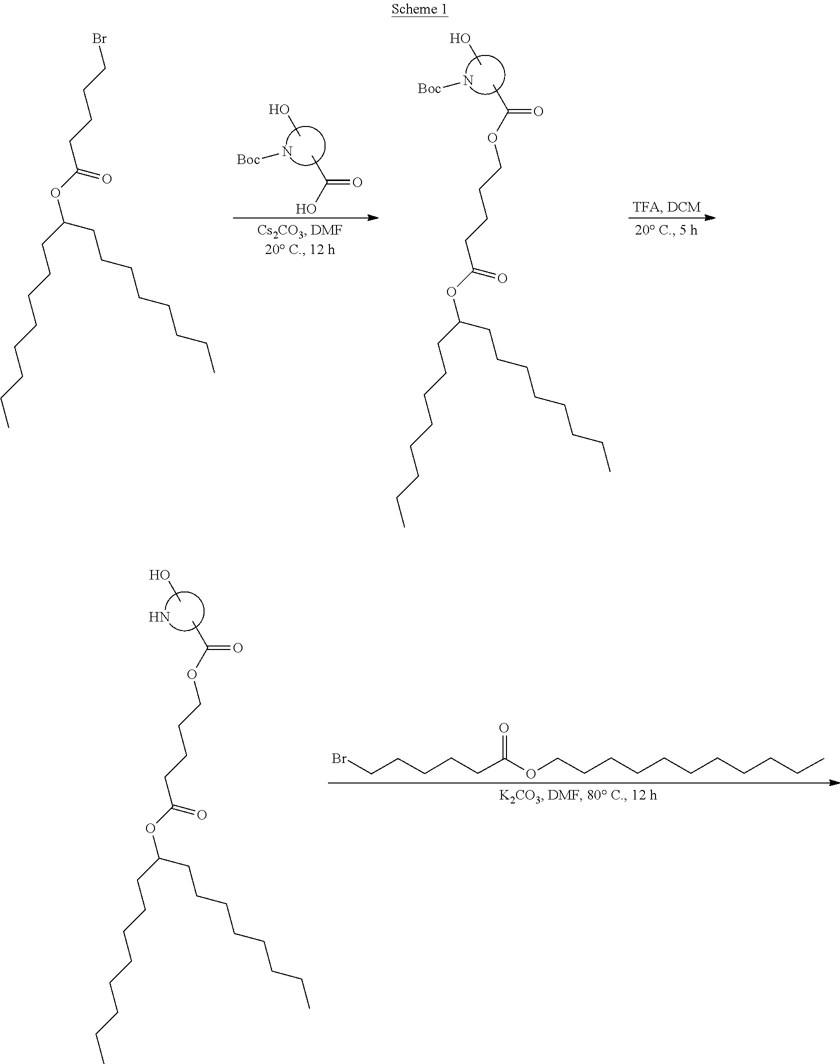

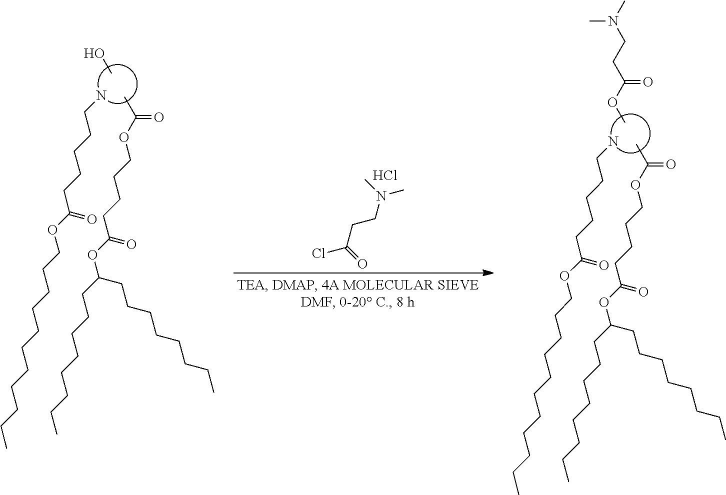

General reaction schemes for synthesis of exemplary ionizable lipid compounds, containing cycloalkyl core structure is shown in Scheme 2.

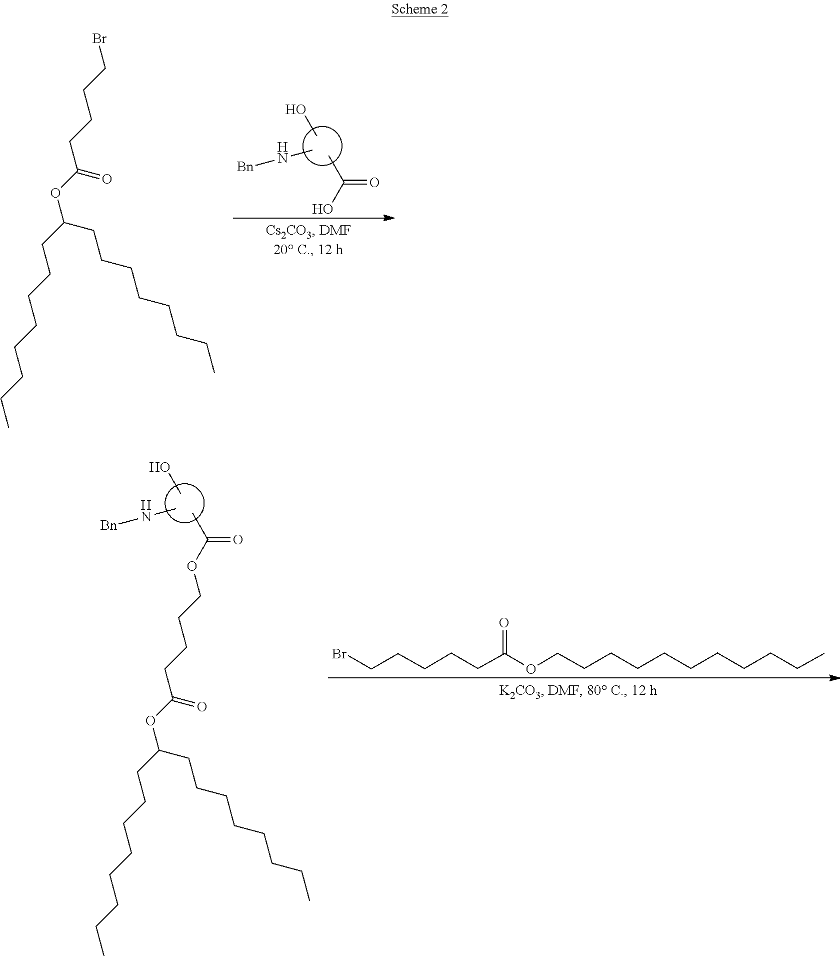

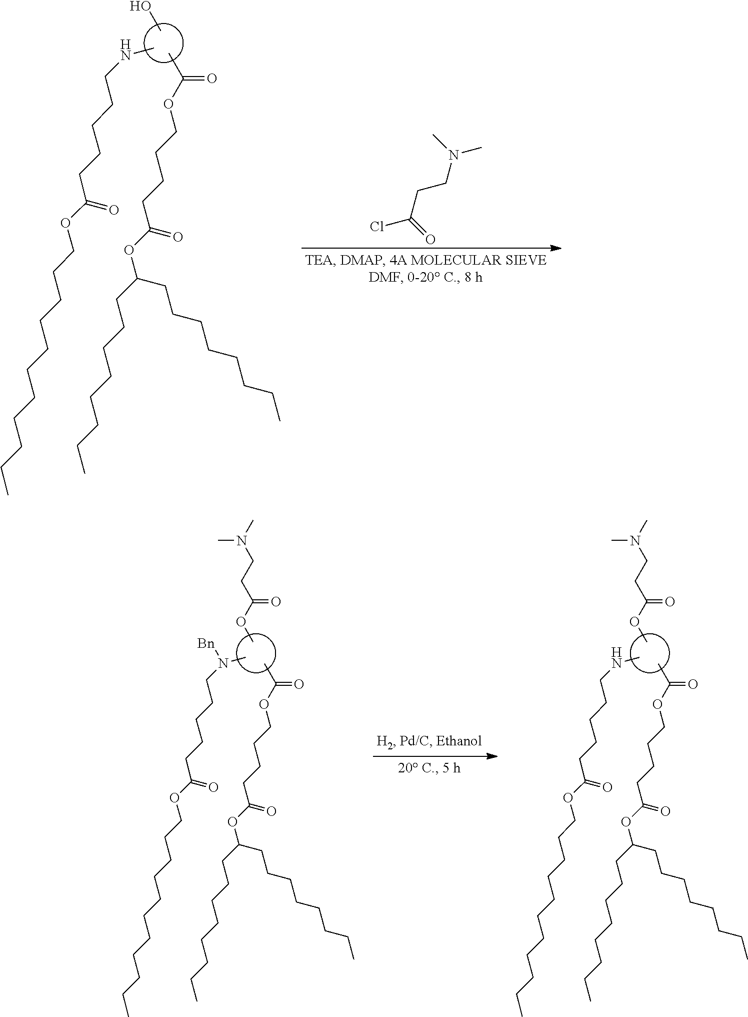

In Scheme 1 or 2, non-limiting examples for the alternative starting heterocyclic or cycloalkyl core structure to prepare exemplary ionizable lipid compounds are shown in Scheme 3.

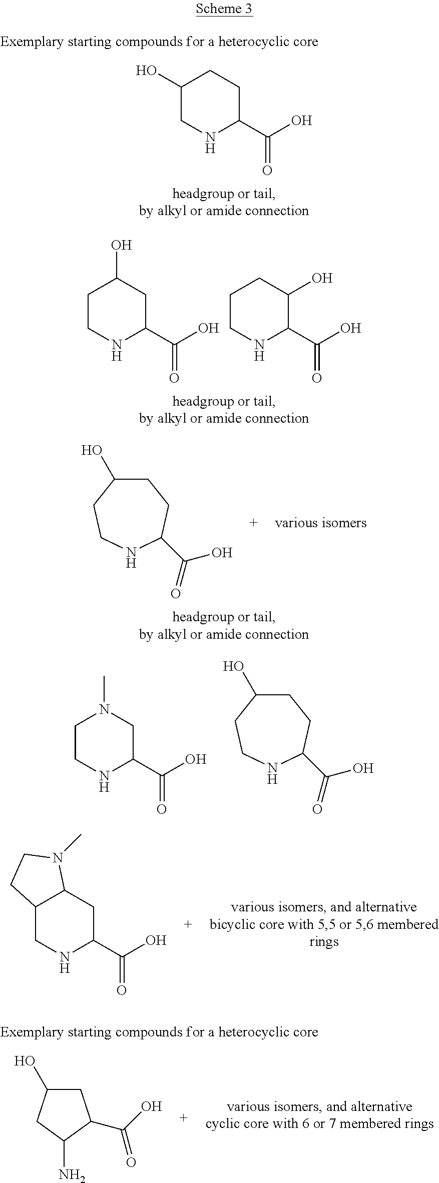

### Example 6. Preparation of Lipid Nanoparticle Compositions with or without a Cargo

### Exemplary Lipid Nanoparticle Compositions

Exemplary lipid nanoparticle compositions were prepared to result in an ionizable lipid:structural lipid:sterol:PEG-lipid at a molar ratio of 50:10:38.5:1.5, respectively. For instance, exemplary lipid nanoparticle compositions in this example are shown in the below chart. The exemplary ionizable lipids used for each exemplary lipid nanoparticle composition were Compounds 2230, 2231, 2260, and 2270 (LNP 2230, LNP 2231, LNP 2260, LNP 2270).

| Lipids | Molar ratios |
|---|---|
| Exemplary ionizable lipid | 50 |
| DSPC | 10 |
| Cholesterol | 38.5 |
| DMPE-PEG2k | 1.5 |

To prepare these compositions, the lipids according to the above chart were solubilized in ethanol, mixed at the above molar ratios, and diluted in ethanol (organic phase) to obtain total lipid concentration of 5.5 mM.

### Comparative Lipid Nanoparticle Compositions

A lipid nanoparticle composition containing C12-200 (LNP C12-200), as control, was prepared to result in C12-200:DOPE:cholesterol (14:0): DMPE-PEG2k) at a molar ratio of 35:16:46.5:2.5, respectively. C12-200 was commercially available ionizable lipid and has a chemical name of 1,1′-((2-(4-(2-((2-(bis(2-hydroxydodecyl)amino)ethyl)(2-hydroxydodecyl) amino)ethyl)piperazin-1-yl)ethyl)azanediyl)bis(dodecan-2-ol). Lipids were solubilized in ethanol. These lipids are mixed at the above-indicated molar ratios and diluted in ethanol (organic phase) to obtain total lipid concentration of 5.5 mM.

Another lipid nanoparticle composition containing MC3 (LNP MC3), as control, was prepared, prepared to result in MC3:DSPC:cholesterol:14:0 DMPE-PEG2k at a molar ratio of 50:38.5:10:1.5, respectively. MC3 was commercially available ionizable lipid having a chemical name of (6Z,9Z,28Z,31Z)-heptatriacont-6,9,28,31-tetraene-19-yl 4-(dimethylamino)butanoate. Lipids are solubilized in ethanol. Lipids were solubilized in ethanol. These lipids are mixed at the above-indicated molar ratios and diluted in ethanol (organic phase) to obtain total lipid concentration of 5.5 mM.

### Lipid Nanoparticle Compositions Encapsulating mRNA

An mRNA solution (aqueous phase, fluc:EPO mRNA) was prepared with RNAse-free water and 100 mM citrate buffer pH 3 for a final concentration of 50 mM citrate buffer and 0.167 mg/mL mRNA concentration (1:1 Fluc:EPO). The formulations were maintained at an ionizable lipid to mRNA at an ionizable lipid nitrogen:mRNA phosphate (N:P) ratio of 15:1 for the LNP C12-200 control, and at an ionizable lipid nitrogen:mRNA phosphate (N:P) ratio of 6:1 for the exemplary lipid nanoparticle compositions (LNP 2230, LNP 2231, LNP 2260, LNP 2270) and for the LNP MC3 control.

For each LNP composition, the lipid mix and mRNA solution were mixed at a 1:3 ratio by volume, respectively, on a NanoAssemblr Ignite (Precision Nanosystems) at a total flow rate of 9 mL/min. The resulting compositions were then loaded into Slide-A-Lyzer G2 dialysis cassettes (10k MWCO) and dialyzed in 200 times sample volume of 1×PBS for 2 hours at room temperature with gentle stirring. The PBS was refreshed, and the compositions were further dialyzed for at least 14 hours at 4° C. with gentle stirring. The dialyzed compositions were then collected and concentrated by centrifugation at 2000×g using Amicon Ultra centrifugation filters (100k MWCO). The concentrated particles were characterized for size, polydispersity, and particle concentration using Zetasizer Ultra (Malvern Panalytical) and for mRNA encapsulation efficiency using Quant-iT RiboGreen RNA Assay Kit (ThermoFisher Scientific).

For pKa measurement, a TNS assay was conducted according to those described in Sabnis et al., Molecular Therapy, 26(6):1509-19), which is incorporated herein by reference in its entirety. Briefly, 20 buffers (10 mM sodium phosphate, 10 mM sodium borate, 10 mM sodium citrate, and 150 mM sodium chloride, in distilled Water) of unique pH values ranging from 3.0-12.0 were prepared using 1M sodium hydroxide and IM hydrochloric acid. 3.25 μL of a LNP composition (0.04 mg/mL mRNA, in PBS) was incubated with 2 μL of TNS reagent (0.3 mM, in DMSO) and 90 μL of buffer for each pH value (described above) in a 96-well black-walled plate. Each pH condition was performed in triplicate wells. The TNS fluorescence was measured using a Biotek Cytation Plate reader at excitation/emission wavelengths of 321/445 nm. The fluorescence values were then plotted and fit using a 4-parameter sigmoid curve. From the fit, the pH value yielding the half-maximal fluorescence was calculated and reported as the apparent LNP pKa value.

The particle characterization data for each exemplary lipid nanoparticle compositions (LNP 2230, LNP 2231, LNP 2260, LNP 2270) are shown in the table below.

| | | | % entrapment | |
|---|---|---|---|---|
| LNP | Size (nm) | PDI | efficiency | pKa |
| 2230 | 87.57 | 0.10 | 92.4 | 4.84 |
| 2260 | 62.62 | 0.14 | 90 | 4.72 |
| 2270 | 68.41 | 0.02 | 88.7 | 4.85 |
| 2231 | 91.57 | 0.05 | 93.9 | 5.03 |

### Example 7. In Vivo Bioluminescent Imaging

The exemplary lipid nanoparticle composition (LNP 2230, LNP 2231, LNP 2260, LNP 2270) and comparative lipid nanoparticle composition (LNP C12-200 and LNP MC3) prepared according to Example 6, with encapsulating an mRNA (EPO), were used in this example.

### Bioluminescence Screening

8-9 week old female Balb/c mice were utilized for bioluminescence-based ionizable lipid screening efforts. Mice were obtained from Jackson Laboratories (JAX Stock: 000651) and allowed to acclimate for one week prior to manipulations. Animals were placed under a heat lamp for a few minutes before introducing them to a restraining chamber. The tail was wiped with alcohol pads (Fisher Scientific) and, for each LNP composition described above, 100 μL of a lipid nanoparticle composition containing 10 μg total mRNA (5 μg Fluc+5 μg EPO) was injected intravenously using a 29G insulin syringe (Covidien). 4-6 hours post-dose, animals were injected with 200 μL of 15 mg/mL D-Luciferin (GoldBio), and placed in set nose cones inside the IVIS Lumina LT imager (PerkinElmer). LivingImage software was utilized for imaging. Whole body bio-luminescence was captured at auto-exposure after which animals are removed from the IVIS and placed into a CO₂ chamber for euthanasia. Cardiac puncture was performed on each animal after placing it in dorsal recumbency, and blood collection was performed using a 25G insulin syringe (BD). Once all blood samples were collected, tubes are spun at 2000 G for 10 minutes using a tabletop centrifuge and plasma was aliquoted into individual Eppendorf tubes (Fisher Scientific) and stored at −80° C. for subsequent EPO quantification. EPO levels in plasma were determined using EPO MSD kit (Meso Scale Diagnostics).

### hEPO MSD Measurement

The reagents used for measuring hEPO levels included:
MSD wash buffer (#R61AA-1)
MSD EPO Kit (#K151VXK-2)
   MSD GOLD 96 Small Spot Streptavidin Plate
   Diluent 100
   Diluent 3
   Diluent 43
   Calibrator 9
   Capture Ab
   Detection Ab
   MSD GOLD Read Buffer B

General procedure. The Plate was coated. 200 μL of biotinylated capture antibody was added to 3.3 mL of Diluent 100 and was mixed by vortexing. 25 μL of the above solution was added to each well of the provided MSD GOLD Small Spot Streptavidin Plate. The plate was sealed with an adhesive plate seal and incubated with shaking at room temperature for 1 hour or at 2-8° C. overnight. The plate was washed 3 times with at least 150 μL/well of 1× MSD Wash Buffer.

Preparation of Calibrator Standards. The Calibrator vial(s) were brought to room temperature. Each vial of Calibrator was reconstituted by adding 250 μL of Diluent 43 to the glass vial, resulting in a 5× concentrated stock of the Calibrator. The reconstituted Calibrator was inverted at least 3 times, and equilibrated at room temperature for 15-30 minutes and then was vortexed briefly. Calibrator Standard 1 was prepared by adding 50 μL of the reconstituted Calibrator to 200 μL of Diluent 43 and vortexing. Calibrator Standard 2 was prepared by adding 75 μL of Calibrator Standard 1 to 225 μL of Diluent 43 and vortexing. The four-fold serial dilutions were repeated 5 additional times to generate a total of 7 Calibrator Standards. Mix by vortexing between each serial dilution. Diluent 43 was used as Calibrator Standard 8 (zero Calibrator).

Samples and Calibrators additions. 25 μL of Diluent 43 was added to each well. 25 μL of the prepared Calibrator Standard or sample was added to each well. The plate was sealed with an adhesive plate seal, and incubate at room temperature with shaking for 1 hour.

Preparation and addition of the Detection Antibody Solution. The detection antibody solution was provided as a 100× stock solution. The working solution was 1×. 60 μL of the supplied 100× detection antibody was added to 5940 μL of Diluent 3. The plate was washed 3 times with at least 150 μL/well of 1×MSD Wash Buffer. 50 μL of the Detection Antibody Solution prepared above was added to each well. The plate was sealed with an adhesive plate seal, and incubated at room temperature with shaking for 1 hour

Sample reading. The plate was washed 3 times with at least 150 μL/well of 1×MSD Wash Buffer. 150 μL of MSD GOLD Read Buffer B was added to each well. The plate was analyzed on an MSD instrument to read the EPO level.

The average radiance levels determined by the in-vivo bioluminescent imaging for each exemplary lipid nanoparticle compositions (LNP 2230, LNP 2231, LNP 2260, LNP 2270) are shown in the table below.

| | Bioluminescence | | | |
|---|---|---|---|---|
| LNP | Liver | Spleen | Lung | hEPO |
| 2230 | 6.62E+05 | 1.61E+06 | 1.02E+04 | 1.22E+05 |
| 2260 | 6.13E+03 | 9.73E+03 | 1.17E+03 | 2.16E+02 |
| 2270 | 3.02E+03 | 6.45E+03 | 1.13E+03 | 9.09E+01 |
| 2231 | 4.22E+05 | 3.77E+06 | 1.83E+04 | 4.70E+04 |

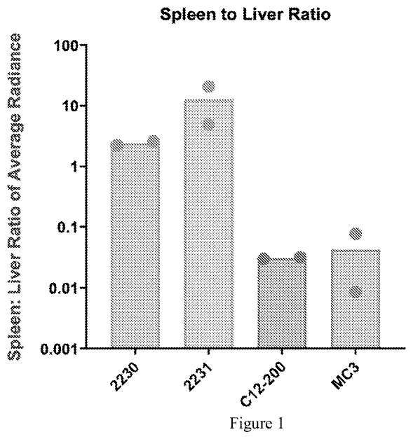

The spleen:liver ratio of average radiance was determined for the exemplary lipid nanoparticle compositions (LNP 2230, LNP 2231), as compared to comparative lipid nanoparticle compositions (LNP C12-200, LNP MC3), and the results are shown in FIG. 1.

As shown in the figure, the exemplary lipid nanoparticle compositions (LNP 2230, LNP 2231) exhibited a significantly higher spleen to liver ratio than that of the comparative lipid nanoparticle compositions (LNP C12-200, LNP MC3) (>>1 v.<<0.1), indicating that instead of standard delivery mostly by liver exhibited for the comparative lipid nanoparticle compositions, the exemplary lipid nanoparticle compositions exhibited surprising high delivery to spleen delivery in addition to liver delivery.

Accordingly, the lipid nanoparticles employing novel ionizable lipids described herein demonstrated selective delivery of the therapeutic cargos outside the liver and, due to the lower lipid levels in the liver, lower liver toxicity is expected.

### Example 8: Synthesis of Exemplary Ionizable Lipid Compounds

### 8.1. Synthesis of Compound 2290

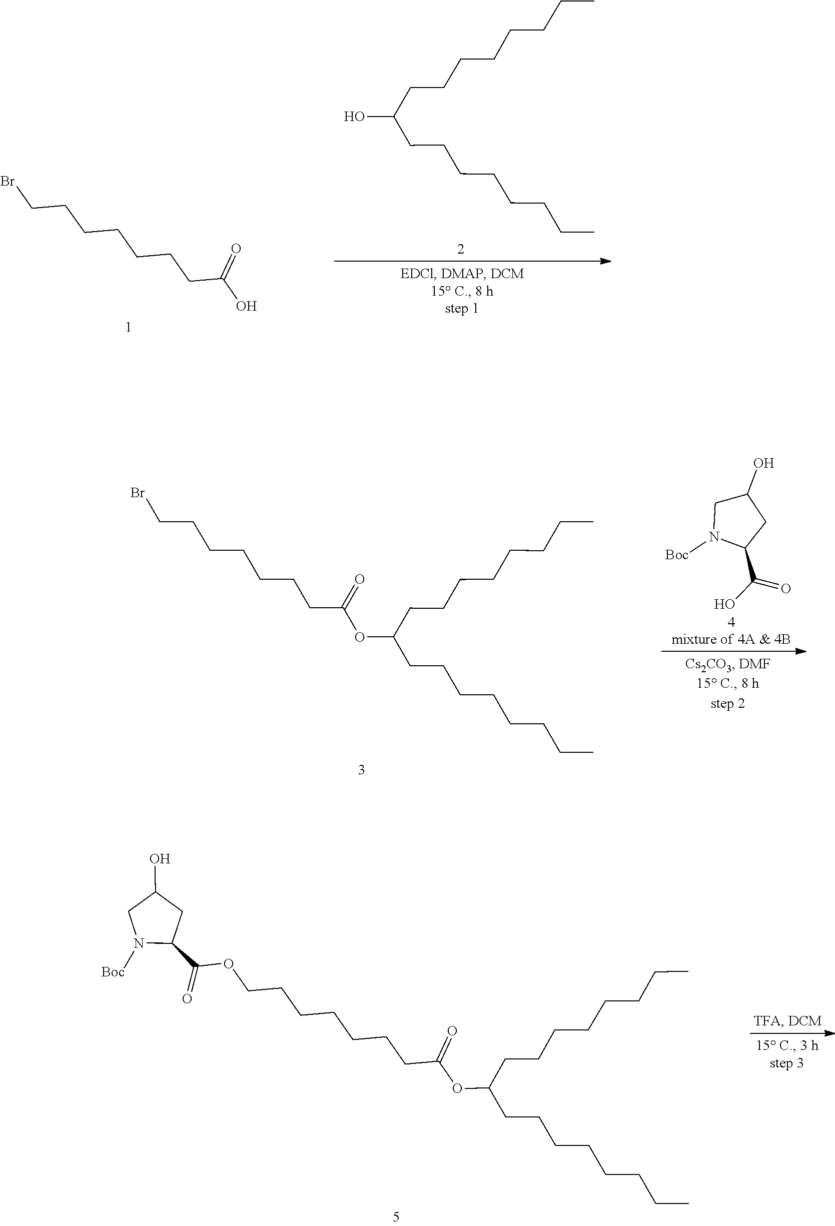 [SMILES: O=C(O)CCCCCCCBr ; CCCCCCCCC(O)CCCCCCCC ; CCCCCCCCC(CCCCCCCC)OC(=O)CCCCCCCBr ; CC(C)(C)OC(=O)N1CC(O)C[C@H]1C(=O)O ; CCCCCCCCC(CCCCCCCC)OC(=O)CCCCCCCOC(=O)[C@@H]1CC(O)CN1C(=O)OC(C)(C)C]

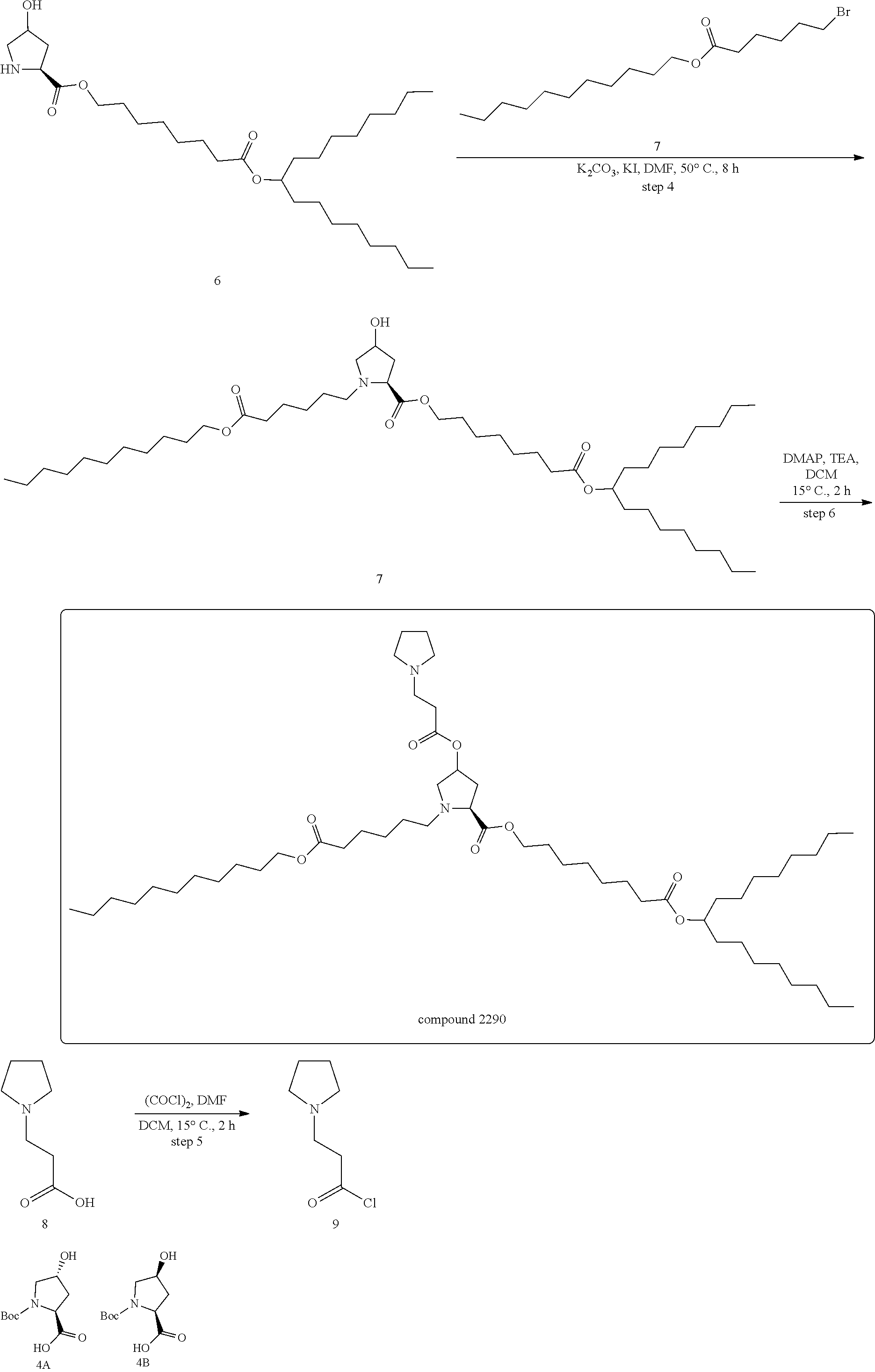

### Step 1

To a solution of 8-bromooctanoic acid (4.35 g, 19.50 mmol, 1 eq) and heptadecan-9-ol (5 g, 19.50 mmol, 1 eq) in DCM (100 mL) was added EDCI (4.48 g, 23.39 mmol, 1.2 eq) and DMAP (1.19 g, 9.75 mmol, 0.5 eq). The mixture was stirred at 15° C. for 8 hours. The reaction mixture was quenched by addition of 200 mL H₂O at 15° C., and then extracted with 600 mL EtOAc (200 mL×3). The combined organic layers were washed with 400 mL brine (200 mL×2), dried over Na₂SO₄, filtered, and concentrated under reduced pressure to give a residue. The residue was purified by column chromatography (SiO₂, petroleum ether/ethyl acetate=1/0 to 20/1) to give 1-octylnonyl 8-bromooctanoate (35 g, 75.83 mmol, 97.24% yield) as colorless oil.

¹H NMR (400 MHz, CDCl₃), 4.84-4.90 (m, 1H), 3.41 (t, J=6.8 Hz, 2H), 2.29 (t, J=7.6 Hz, 2H), 1.82-1.88 (m, 2H), 1.62-1.65 (m, 2H), 1.42-1.52 (m, 6H), 1.25-1.36 (m, 28H), 0.89 (t, J=7.2 Hz, 6H).

### Step 2

A mixture of 1-octylnonyl 8-bromooctanoate (1 g, 2.17 mmol, 1.2 eq), (2S)-1-tert-butoxycarbonyl-4-hydroxy-pyrrolidine-2-carboxylic acid (417.51 mg, 1.81 mmol, 1 eq), Cs₂CO₃ (1.29 g, 3.97 mmol, 2.2 eq) in DMF (10 mL) was degassed and purged with N₂ for 3 times, and then the mixture was stirred at 15° C. for 8 hours under N₂ atmosphere. The reaction mixture was quenched by addition of 50 mL H₂O at 15° C., and then extracted with 150 mL EtOAc (50 mL×3). The combined organic layers were washed with 100 mL brine (50 mL×2), dried over Na₂SO₄, filtered, and concentrated under reduced pressure to give a residue. The residue was purified by column chromatography (SiO₂, petroleum ether/ethyl acetate=20/1 to 3/1) to give O1-tert-butyl O2-[8-(1-octylnonoxy)-8-oxo-octyl](2S)-4-hydroxypyrrolidine-1,2-dicarboxylate (4.55 g, 7.44 mmol, 82.37% yield) as colorless oil.

¹H NMR (400 MHz, CDCl₃), 4.84-4.90 (m, 1H), 4.18-4.52 (m, 3H), 4.06-4.10 (m, 1H), 3.42-3.72 (m, 2H), 2.21-2.39 (m, 3H), 2.07-2.11 (m, 1H), 1.25-1.67 (m, 48H), 0.88 (t, J=6.8 Hz, 6H).

### Step 3

To a solution of O1-tert-butyl O2-[8-(1-octylnonoxy)-8-oxo-octyl](2S)-4-hydroxypyrrolidine-1,2-dicarboxylate (4.5 g, 7.35 mmol, 1 eq) in DCM (30 mL) was added TFA (23.10 g, 202.59 mmol, 15 mL, 27.55 eq). The mixture was stirred at 15° C. for 3 hours. The reaction mixture was quenched by addition of 60 mL aqeuous NaHCO₃ at 15° C., and then extracted with 150 mL EtOAc (50 mL×3). The combined organic layers were washed with 100 mL brine (50 mL×2), dried over Na₂SO₄, filtered, and concentrated under reduced pressure to give [8-(1-octylnonoxy)-8-oxo-octyl](2S)-4-hydroxypyrrolidine-2-carboxylate (3.76 g, 7.35 mmol, 100.00% yield) as colorless oil.

### Step 4

To a solution of [8-(1-octylnonoxy)-8-oxo-octyl](2S)-4-hydroxypyrrolidine-2-carboxylate (2 g, 3.91 mmol, 1 eq) and undecyl 6-bromohexanoate (1.64 g, 4.69 mmol, 1.2 eq) in DMF (40 mL) was added K₂CO₃ (1.62 g, 11.72 mmol, 3 eq) and KI (324.37 mg, 1.95 mmol, 0.5 eq). The mixture was stirred at 50° C. for 8 hours. The reaction mixture was quenched by addition of 50 mL H₂O at 15° C. and extracted with 150 mL EtOAc (50 mL×3). The combined organic layers were washed with 100 mL brine (50 mL×2), dried over Na₂SO₄, filtered, and concentrated under reduced pressure to give a residue. The residue was purified by column chromatography (SiO₂, petroleum ether/ethyl acetate=20/1 to 1/1) to give [8-(1-octylnonoxy)-8-oxo-octyl](2S)-4-hydroxy-1-(6-oxo-6-undecoxy-hexyl)pyrrolidine-2-carboxylate (1.6 g, 2.05 mmol, 52.48% yield) as colorless oil.

¹H NMR (400 MHz, CDCl₃), 4.86-4.90 (m, 1H), 4.24-4.53 (m, 1H), 4.04-4.15 (m, 4H), 2.99-3.69 (m, 2H), 1.84-2.84 (m, 8H), 1.59-1.68 (m, 8H), 1.45-1.54 (m, 6H), 1.15-1.44 (m, 50H), 0.89 (t, J=7.8 Hz, 9H). LCMS: (M+H⁺): 780.4 @13.579 minutes.

### Step 5

To a solution of 3-pyrrolidin-1-ylpropanoic acid (100 mg, 698.41 mol, 1 eq) in DCM (5 mL) was added (COCl)₂ (443.23 mg, 3.49 mmol, 305.68 μL, 5 eq) and DMF (5.10 mg, 69.84 μmol, 5.37 μL, 0.1 eq). The mixture was stirred at 15° C. for 2 hours. The reaction mixture was concentrated under reduced pressure to give 3-pyrrolidin-1-ylpropanoyl chloride (138 mg, 696.65 μmol, 99.75% yield, HCl) as a yellow solid.

### Step 6

To the suspension of [8-(1-octylnonoxy)-8-oxo-octyl](2S)-4-hydroxy-1-(6-oxo-6-undecoxy-hexyl)pyrrolidine-2-carboxylate (200 mg, 256.34 mol, 1 eq), TEA (77.82 mg, 769.03 mol, 107.04 μL, 3 eq) and DMAP (15.66 mg, 128.17 mol, 0.5 eq) in DCM (3 mL) was added dropwise [3-pyrrolidin-1-ylpropanoyl chloride (126.95 mg, 640.85 mol, 2.5 eq, HCl) in DCM (1 mL) at 15° C. The mixture was stirred at 15° C. for 2 hours under N₂ atmosphere. The reaction mixture was quenched by addition of 10 mL saturated NaHCO₃ at 15° C., and then extracted with 30 mL EtOAc (10 mL×3). The combined organic layers were washed with 20 mL brine (10 mL×2), dried over Na₂SO₄, filtered, and concentrated under reduced pressure to give a residue. The residue was purified by prep-TLC (SiO₂, EtOAc:MeOH=10:1) to give [8-(1-octylnonoxy)-8-oxo-octyl](2S)-1-(6-oxo-6-undecoxy-hexyl)-4-(3-pyrrolidin-1-ylpropanoyloxy)pyrrolidine-2-carboxylate (100 mg, 108.24 mol, 49.00% yield, 98% purity) as colorless oil.

¹H NMR (400 MHz, CDCl₃), 5.19-5.31 (m, 1H), 4.84-4.89 (m, 1H), 4.04-4.15 (m, 4H), 3.43-3.55 (m, 1H), 3.09-3.26 (m, 1H), 2.49-2.81 (m, 10H), 2.24-2.36 (m, 5H), 1.95-2.22 (m, 1H), 1.80 (s, 3H), 1.59-1.68 (m, 8H), 1.44-1.54 (m, 6H), 1.12-1.42 (m, 50H), 0.85-0.93 (m, 9H). LCMS: (M+H⁺): 905.4 @1.950/2.035 minutes.

### 8.2. Synthesis of Compound 2291

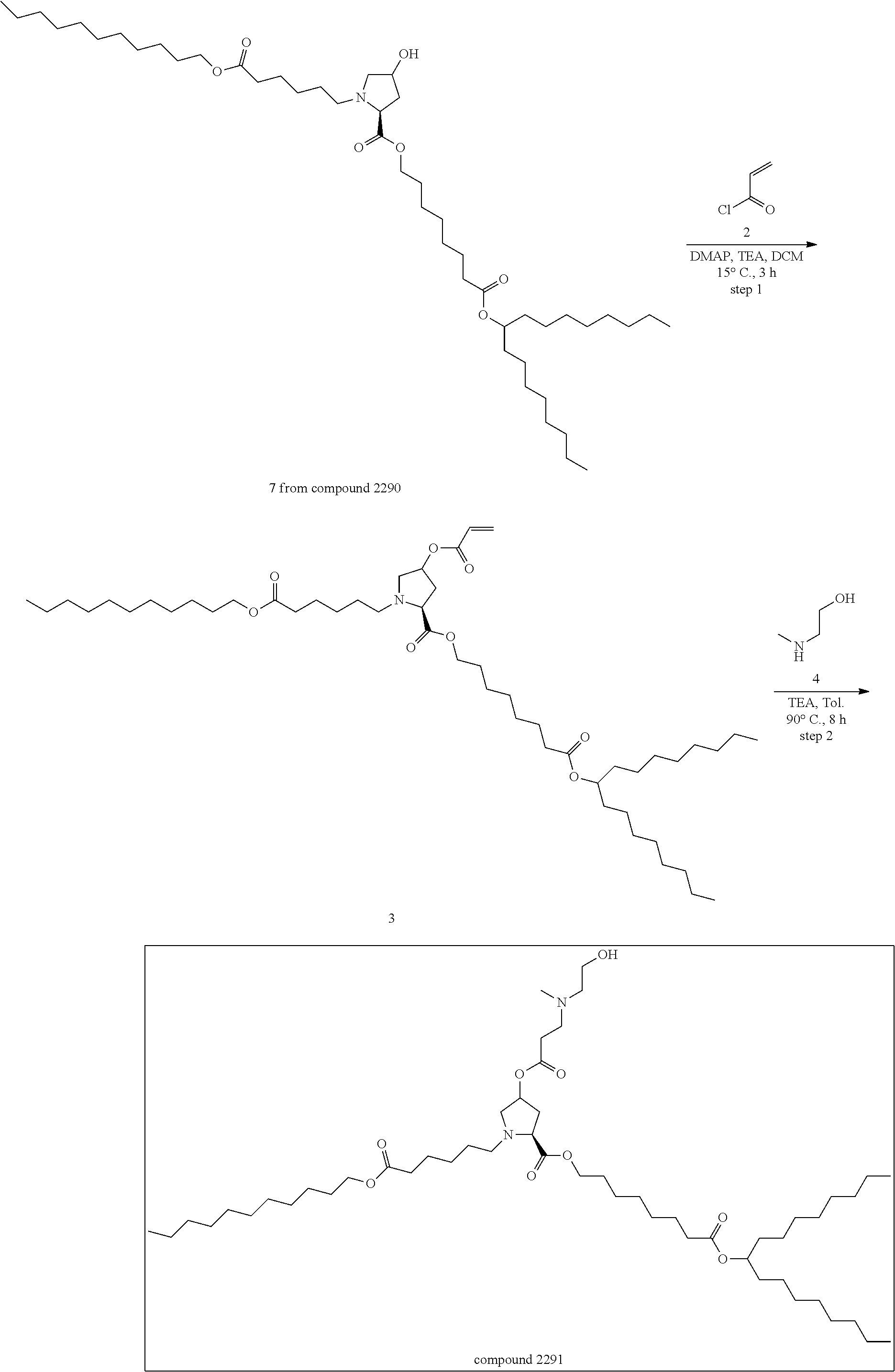

### Step 1

To the suspension of [8-(1-octylnonoxy)-8-oxo-octyl](2S)-4-hydroxy-1-(6-oxo-6-undecoxy-hexyl)pyrrolidine-2-carboxylate (1 g, 1.28 mmol, 1 eq), TEA (648.47 mg, 6.41 mmol, 891.99 uL, 5 eq) and DMAP (78.29 mg, 640.85 mol, 0.5 eq) in DCM (9 mL) was added dropwise a solution of prop-2-enoyl chloride (464.02 mg, 5.13 mmol, 418.04 μL, 4 eq) in DCM (3 mL). The mixture was stirred at 15° C. for 3 hours under N₂ atmosphere. The reaction mixture was quenched by addition of 10 mL H₂O at 15° C., and then extracted with 30 mL EtOAc (10 mL×3). The combined organic layers were washed with 20 mL brine (10 mL×2), dried over Na₂SO₄, filtered, and concentrated under reduced pressure to give a residue. The residue was purified by column chromatography (SiO₂, petroleum ether/ethyl acetate=20/1 to 8/1) to give [8-(1-octylnonoxy)-8-oxo-octyl](2S)-1-(6-oxo-6-undecoxy-hexyl)-4-prop-2-enoyloxy-pyrrolidine-2-carboxylate (300 mg, 359.60 mol, 28.06% yield) as colorless oil.

### Step 2

A mixture of [8-(1-octylnonoxy)-8-oxo-octyl](2S)-1-(6-oxo-6-undecoxy-hexyl)-4-prop-2-enoyloxy-pyrrolidine-2-carboxylate (300 mg, 359.60 mol, 1 eq), 2-(methylamino)ethanol (27.01 mg, 359.60 mol, 28.89 μL, 1 eq) in toluene (3 mL) was degassed and purged with N₂ for 3 times, and then the mixture was stirred at 90° C. for 8 hours under N₂ atmosphere. The reaction mixture was concentrated under reduced pressure to give a residue. The residue was purified by prep-HPLC (column: Phenomenex Luna C18 100×30 mm×5 m; mobile phase: [water(HCl)-ACN]; B %: 45%-75%, 10 minutes) to get a solution. The solution was adjusted pH=˜7 with saturated NaHCO₃, extracted with EtOAc (20 mL×3). The organic layer was washed with brine (20 mL×2), dried over Na₂SO₄, filtered, and the filtrate was concentrated under reduced pressure to give [8-(1-octylnonoxy)-8-oxo-octyl](2S)-4-[3-[2-hydroxyethyl(methyl)amino]propanoyloxy]-1-(6-oxo-6-undecoxy-hexyl)pyrrolidine-2-carboxylate (30 mg, 32.59 mol, 9.06% yield, 98.8% purity) as colorless oil.

¹H NMR (400 MHz, CDCl₃), 5.15-5.31 (m, 1H), 4.81-4.93 (m, 1H), 4.01-4.18 (m, 4H), 3.39-3.72 (m, 3H), 3.04-3.31 (m, 1H), 1.97-2.88 (m, 18H), 1.59-1.66 (m, 8H), 1.47-1.55 (m, 6H), 1.23-1.38 (m, 48H), 0.84-0.95 (m, 9H). LCMS: (M+H⁺): 909.7 @9.772 minutes.

### 8.3. Synthesis of Compound 2292

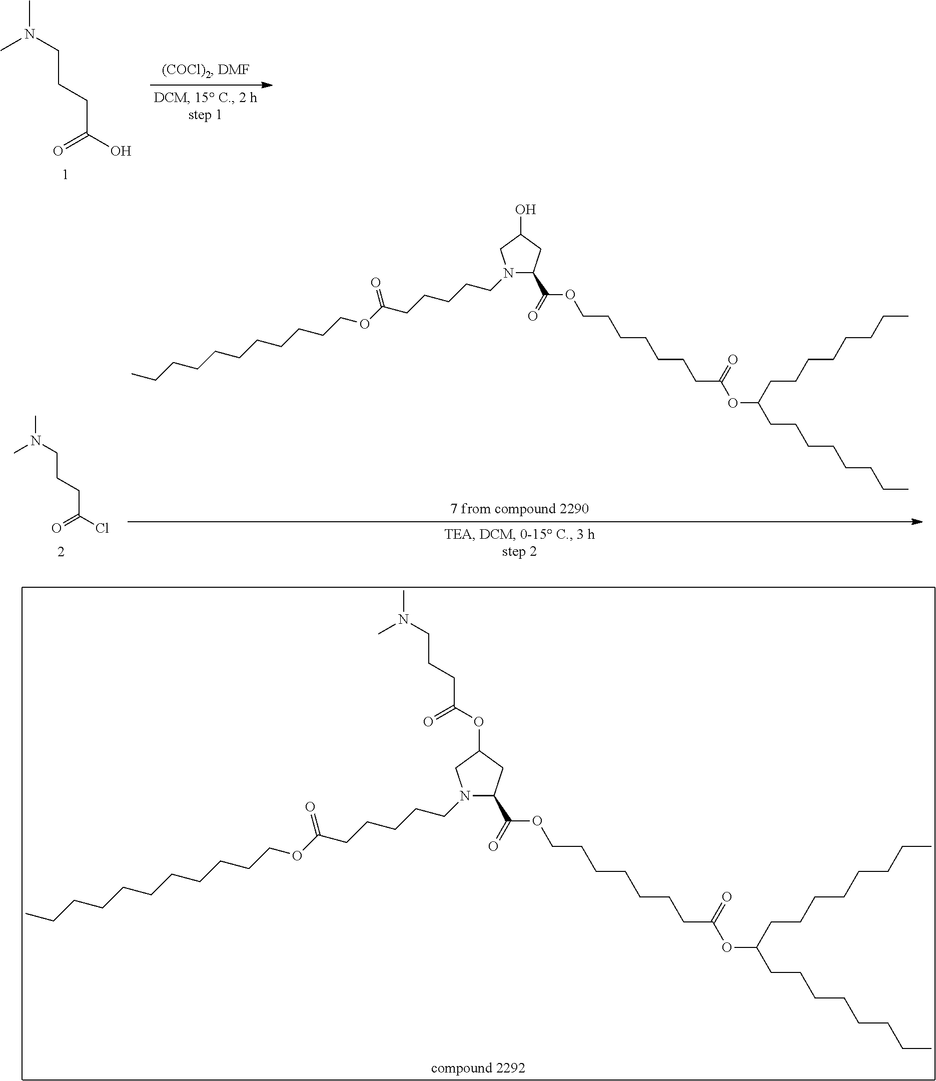

### Step 1

To a solution of 3-(dimethylamino)propanoic acid (100 mg, 651.01 mol, 1 eq, HCl) in DCM (5 mL) was added (COCl)₂ (354.59 mg, 2.79 mmol, 244.55 μL, 4 eq) and DMF (5.10 mg, 69.84 mol, 5.37 μL, 0.1 eq). The mixture was stirred at 15° C. for 2 hours. The mixture was concentrated under reduced pressure to give 3-(dimethylamino)propanoyl chloride (112 mg, 650.96 mol, 99.99% yield, HCl) as a yellow solid.

### Step 2

To a solution of [8-(1-octylnonoxy)-8-oxo-octyl](2S)-4-hydroxy-1-(6-oxo-6-undecoxy-hexyl)pyrrolidine-2-carboxylate (200 mg, 256.34 mol, 1 eq) in DCM (10 mL) was added TEA (129.69 mg, 1.28 mmol, 178.40 μL, 5 eq) and 3-(dimethylamino)propanoyl chloride (112 mg, 650.96 mol, 2.54 eq, HCl) at 0° C. The mixture was stirred at 15° C. for 3 hours.

The reaction mixture was quenched by addition of 10 mL NaHCO₃ at 15° C. and then extracted with 30 mL EtOAc (10 mL×3). The combined organic layers were washed with 20 mL brine (10 mL×2), dried over Na₂SO₄, filtered, and concentrated under reduced pressure to give a residue. The residue was purified by prep-TLC (SiO₂, EtOAc:MeOH=10:1) to give [8-(1-octylnonoxy)-8-oxo-octyl](2S)-4-[4-(dimethylamino)butanoyloxy]-1-(6-oxo-6-undecoxy-hexyl)pyrrolidine-2-carboxylate (33 mg, 36.42 mol, 14.7% yield, 98.6% purity) as colorless oil.

¹H NMR (400 MHz, CDCl₃), 5.11-5.34 (m, 1H), 4.84-4.90 (m, 1H), 4.03-4.14 (m, 4H), 3.43-3.54 (m, 1H), 3.09-3.25 (m, 1H), 2.03-2.77 (m, 19H), 1.77-1.82 (m, 2H), 1.60-1.65 (m, 8H), 1.49-1.52 (m, 6H), 1.27-1.34 (m, 48H), 0.87-0.90 (m, 9H).

LCMS: (M/2+1): 893.4 @10.022 minutes.

### 8.4. Synthesis of Compound 2293

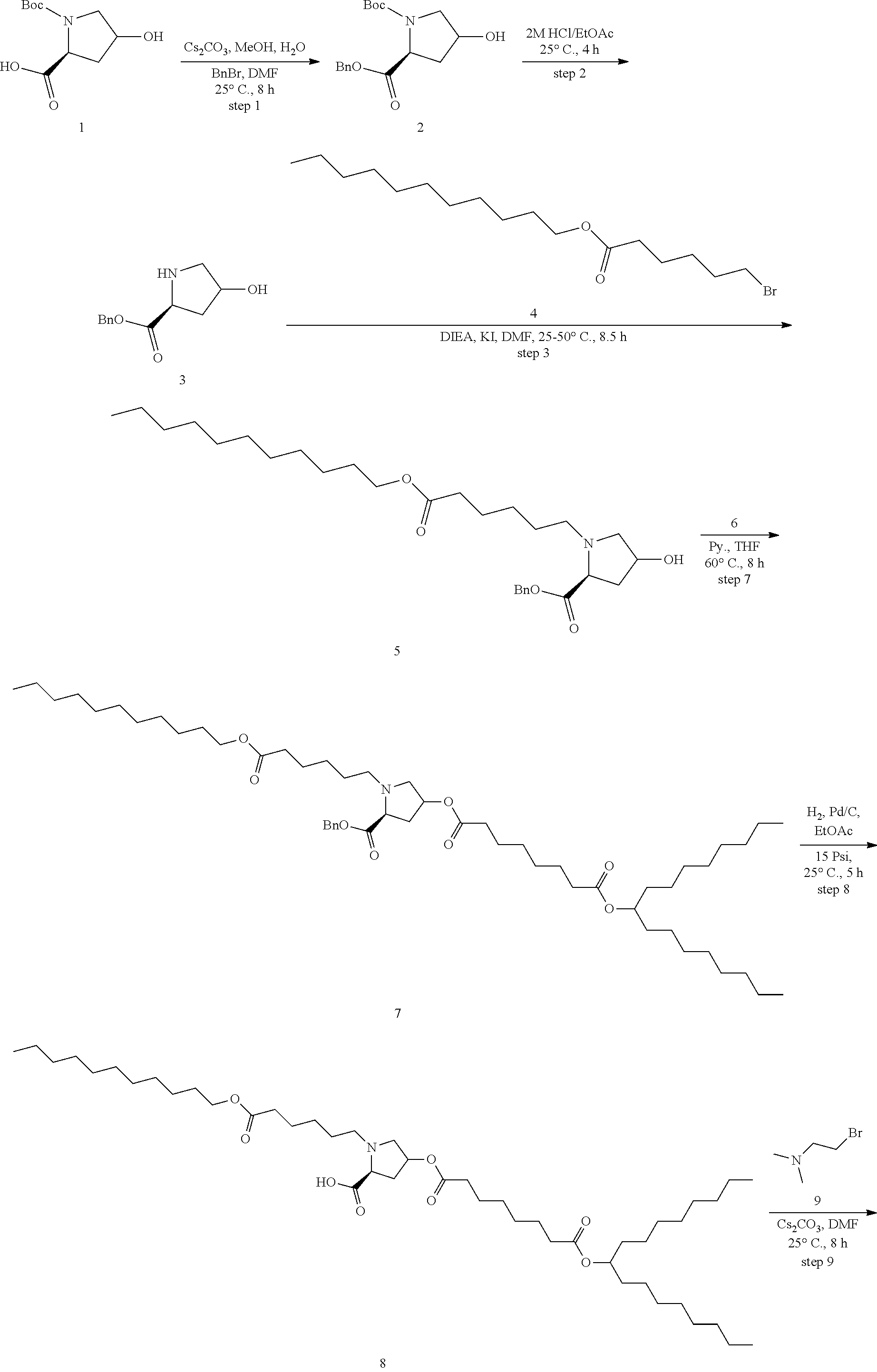

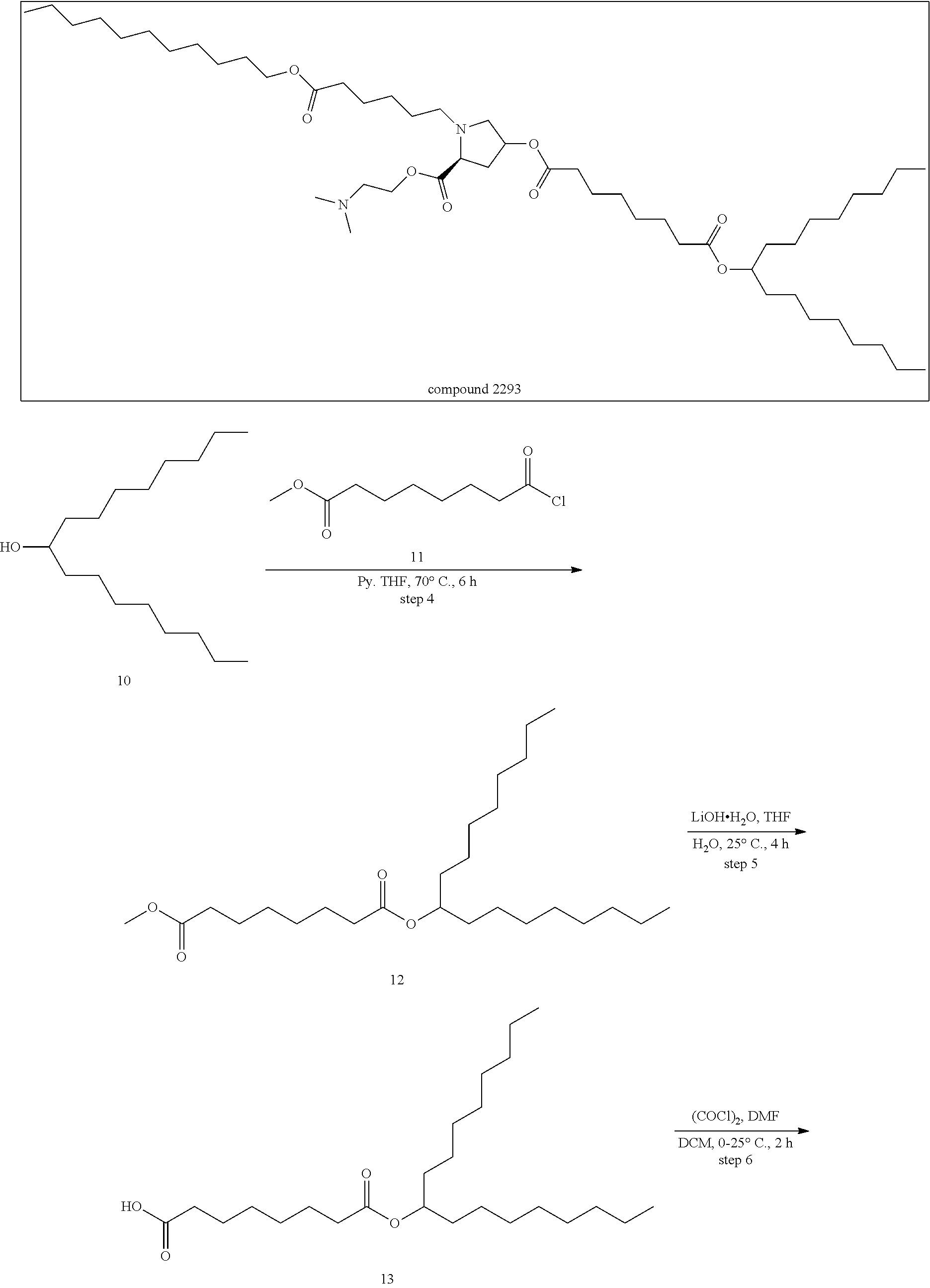

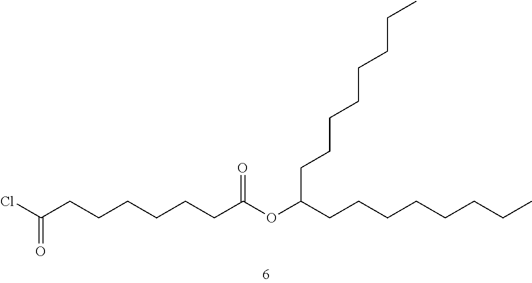 [SMILES: CCCCCCCCC(CCCCCCCC)OC(=O)CCCCCCC(=O)Cl]

### Step 1

A solution of (2S)-1-tert-butoxycarbonyl-4-hydroxy-pyrrolidine-2-carboxylic acid (10 g, 43.24 mmol, 1 eq) in MeOH (50 mL) and H₂O (20 mL) was adjusted to pH=7.0 with dicesium carbonate (8.45 g, 25.95 mmol, 0.6 eq). The mixture was concentrated under reduced pressure to give a residue. The residue was dissolved in DMF (100 mL), then BnBr (7.40 g, 43.24 mmol, 5.14 mL, 1 eq) was added at 25° C. The mixture was degassed and purged with N₂ for 3 times, and then stirred at 25° C. for 8 hours under N₂ atmosphere. The reaction mixture was diluted with 50 mL H₂O, extracted with 300 mL EtOAc (100 mL×3). The combined organic layers were washed with 100 mL brine, dried over Na₂SO₄, filtered, and concentrated under reduced pressure to give a residue. The residue was purified by column chromatography (SiO₂, petroleum ether/ethyl acetate=8/1 to 2/1) to give O2-benzyl O1-tert-butyl (2S)-4-hydroxypyrrolidine-1,2-dicarboxylate (13 g, 40.45 mmol, 93.54% yield) as colourless oil.

¹H NMR (400 MHz, CDCl₃), 7.31-7.40 (m, 5H), 4.99-5.16 (m, 3H), 4.23-4.29 (m, 2H), 3.35-3.55 (m, 1H), 3.10-3.30 (m, 1H), 2.10-2.40 (m, 1H), 1.80-1.90 (m, 1H), 1.20-1.40 (m, 9H).

### Step 2

To a solution of O2-benzyl O1-tert-butyl (2S)-4-hydroxypyrrolidine-1,2-dicarboxylate (13 g, 40.45 mmol, 1 eq) in EtOAc (80 mL) was added dropwise HCl/EtOAc (4 M, 80 mL, 7.91 eq) at 20° C. The mixture was stirred at 25° C. for 4 hours under N₂ atmosphere. The reaction mixture was filtered, and the residue was collected and concentrated under reduced pressure to give benzyl (2S)-4-hydroxypyrrolidine-2-carboxylate (9 g, 34.92 mmol, 86.33% yield, HCl) as a white solid.

### Step 3

To a solution of benzyl (2S)-4-hydroxypyrrolidine-2-carboxylate (1 g, 3.88 mmol, 1 eq, HCl) in DMF (70 mL) was added DIEA (1.00 g, 7.76 mmol, 1.35 mL, 2 eq) at 25° C. and stirred for 0.5 hour under N₂ atmosphere. The mixture was added undecyl 6-bromohexanoate (1.36 g, 3.88 mmol, 1 eq) and KI (128.83 mg, 776.06 mol, 0.2 eq) and stirred at 50° C. for 8 hours under N₂ atmosphere. The reaction mixture was diluted with 50 mL H₂O, extracted with 300 mL EtOAc (150 mL×2). The combined organic layers were washed with 200 mL brine (100 mL×2), dried over Na₂SO₄, filtered, and concentrated under reduced pressure to give a residue. The residue was purified by column chromatography (SiO₂, petroleum ether/ethyl acetate=20/1 to 5/1) to give benzyl (2S)-4-hydroxy-1-(6-oxo-6-undecoxy-hexyl)pyrrolidine-2-carboxylate (2.8 g, 5.72 mmol, 49.12% yield) as colourless oil.

¹H NMR (400 MHz, CDCl₃), 7.25-7.55 (m, 5H), 5.14-5.25 (m, 2H), 4.25˜4.48 (m, 1H), 4.05 (t, J=6.8 Hz, 2H), 3.15-3.65 (m, 2H), 2.55-2.75 (m, 1H), 2.40-2.55 (m, 1H), 2.30-2.40 (m, 1H), 2.20-2.30 (m, 2H), 2.05-2.15 (m, 1H), 1.90-2.00 (m, 1H), 1.55-1.75 (m, 4H), 1.40-1.50 (m, 2H), 1.20-1.35 (m, 18H), 0.89 (t, J=6.4 Hz, 3H).

### Step 4

A mixture of methyl 8-chloro-8-oxo-octanoate (3.22 g, 15.60 mmol, 2.21 mL, 1 eq), heptadecan-9-ol (4 g, 15.60 mmol, 1 eq) and pyridine (1.23 g, 15.60 mmol, 1.26 mL, 1 eq) in THF (20 mL) was stirred at 70° C. for 6 hours under N₂ atmosphere. The reaction mixture was filtered and concentrated under reduced pressure to give a residue. The residue was purified by column chromatography (SiO₂, petroleum ether/ethyl acetate=1/0 to 20/1) to give O1-methyl 08-(1-octylnonyl) octanedioate (3.6 g, 8.44 mmol, 54.10% yield) as colourless oil.

### Step 5

To a solution of O1-methyl 08-(1-octylnonyl) octanedioate (3.6 g, 8.44 mmol, 1 eq) in THF (15 mL) was added dropwise LiOH·H₂O (424.88 mg, 10.12 mmol, 1.2 eq) in H₂O (1 mL) at 25° C. The mixture was stirred at 25° C. for 4 hours under N₂ atmosphere. The reaction mixture was diluted with 500 mL H₂O, extracted with 60 mL EtOAc (30 mL×2). The combined organic layers were dried over Na₂SO₄, filtered, and concentrated under reduced pressure to give a residue. The residue was purified by column chromatography (SiO₂, petroleum ether/ethyl acetate=10/1 to 5/1) to give 8-(1-octylnonoxy)-8-oxo-octanoic acid (2.1 g, 5.09 mmol, 60.32% yield) as colourless oil.

### Step 6

To a solution of 8-(1-octylnonoxy)-8-oxo-octanoic acid (2.1 g, 5.09 mmol, 1 eq) in DCM (25 mL) was added dropwise (COCl)₂ (3.23 g, 25.45 mmol, 2.23 mL, 5 eq) and DMF (37.20 mg, 508.91 μmol, 39.15 μL, 0.1 eq) at 0° C. The mixture was stirred at 25° C. for 2 hours under N₂ atmosphere. The reaction mixture was concentrated under reduced pressure to give crude product 1-octylnonyl 8-chloro-8-oxo-octanoate (2.3 g, crude) as colorless oil and used into the next step without further purification.

### Step 7

A mixture of benzyl (2S)-4-hydroxy-1-(6-oxo-6-undecoxy-hexyl)pyrrolidine-2-carboxylate (0.4 g, 816.85 μmol, 1 eq), 1-octylnonyl 8-chloro-8-oxo-octanoate (704.27 mg, 1.63 mmol, 2 eq) and pyridine (64.61 mg, 816.85 mol, 65.93 μL, 1 eq) in THF (15 mL) was stirred at 60° C. for 8 hours under N₂ atmosphere. The reaction mixture was filtered, and the filtrate was diluted with 10 mL H₂O, then extracted with 60 mL EtOAc (30 mL×2). The combined organic layers were dried over Na₂SO₄, filtered, and concentrated under reduced pressure to give a residue. The residue was purified by column chromatography (SiO₂, petroleum ether/ethyl acetate=1/0 to 10/1) to give O1-[(5S)-5-benzyloxycarbonyl-1-(6-oxo-6-undecoxy-hexyl)pyrrolidin-3-yl]O8-(1-octylnonyl) octanedioate (330 mg, 373.17 mol, 45.68% yield) as colourless oil.

¹H NMR (400 MHz, CDCl₃), 7.31-7.39 (m, 5H), 5.14-5.25 (m, 3H), 4.85-4.89 (m, 1H), 4.06 (t, J=6.8 Hz, 2H), 3.10-3.55 (m, 2H), 2.20-2.75 (m, 11H), 2.05-2.20 (m, 1H), 1.58-1.75 (m, 6H), 1.40-1.55 (m, 6H), 1.20-1.35 (m, 48H), 0.89 (t, J=6.4 Hz, 9H).

### Step 8

To a solution of Pd/C (500 mg, 10% purity) in EtOAc (400 mL) was added O1-[(5S)-5-benzyloxycarbonyl-1-(6-oxo-6-undecoxy-hexyl)pyrrolidin-3-yl]O8-(1-octylnonyl) octanedioate (500 mg, 565.41 mol, 1 eq). The mixture was stirred at 25° C. for 5 hours under H₂ under 15 Psi. The mixture was filtered, and the filtrate was concentrated under reduced pressure to give (2S)-4-[8-(1-octylnonoxy)-8-oxo-octanoyl]oxy-1-(6-oxo-6-undecoxy-hexyl)pyrrolidine-2-carboxylic acid (350 mg, crude) as colorless oil.

### Step 9

To a solution of (2S)-4-[8-(1-octylnonoxy)-8-oxo-octanoyl]oxy-1-(6-oxo-6-undecoxy-hexyl) pyrrolidine-2-carboxylic acid (200 mg, 251.83 mol, 1 eq) and Cs₂CO₃ (164.10 mg, 503.66 μmol, 2 eq) in DMF (5 mL) was added 2-bromo-N,N-dimethyl-ethanamine (45.94 mg, 302.19 mol, 1.2 eq). The mixture was stirred at 25° C. for 8 hours. The mixture was added into H₂O (10 mL), extracted with EtOAc (10 mL×3). The organic layer was washed with brine (10 mL×2), dried over Na₂SO₄, filtered, and concentrated under reduced pressure. The residue was purified by column chromatography (SiO₂, petroleum ether/ethyl acetate=10/1 to 0/1) and by prep-TLC (SiO₂, ethyl acetate:MeOH=1:0, added 1% NH₃·H₂O) to give O1-[(5S)-5-[2-(dimethylamino)ethoxycarbonyl]-1-(6-oxo-6-undecoxy-hexyl)pyrrolidin-3-yl]O8-(1-octylnonyl) octanedioate (125 mg, 143.01 mol, 56.79% yield, 99% purity) as colorless oil.

¹H NMR (400 MHz, CDCl₃), 5.14-5.25 (m, 1H), 4.85-4.89 (m, 1H), 4.23-4.28 (m, 2H), 4.05 (t, J=6.8 Hz, 2H), 3.10-3.60 (m, 2H), 2.25-2.80 (m, 18H), 2.00-2.10 (m, 1H), 1.58-1.65 (m, 8H), 1.40-1.55 (m, 6H), 1.20-1.35 (m, 46H), 0.89 (t, J=6.4 Hz, 9H).

LCMS: (M+H⁺): 865.7 @10.196/10.709 minutes.

### 8.5. Synthesis of Compound 2294

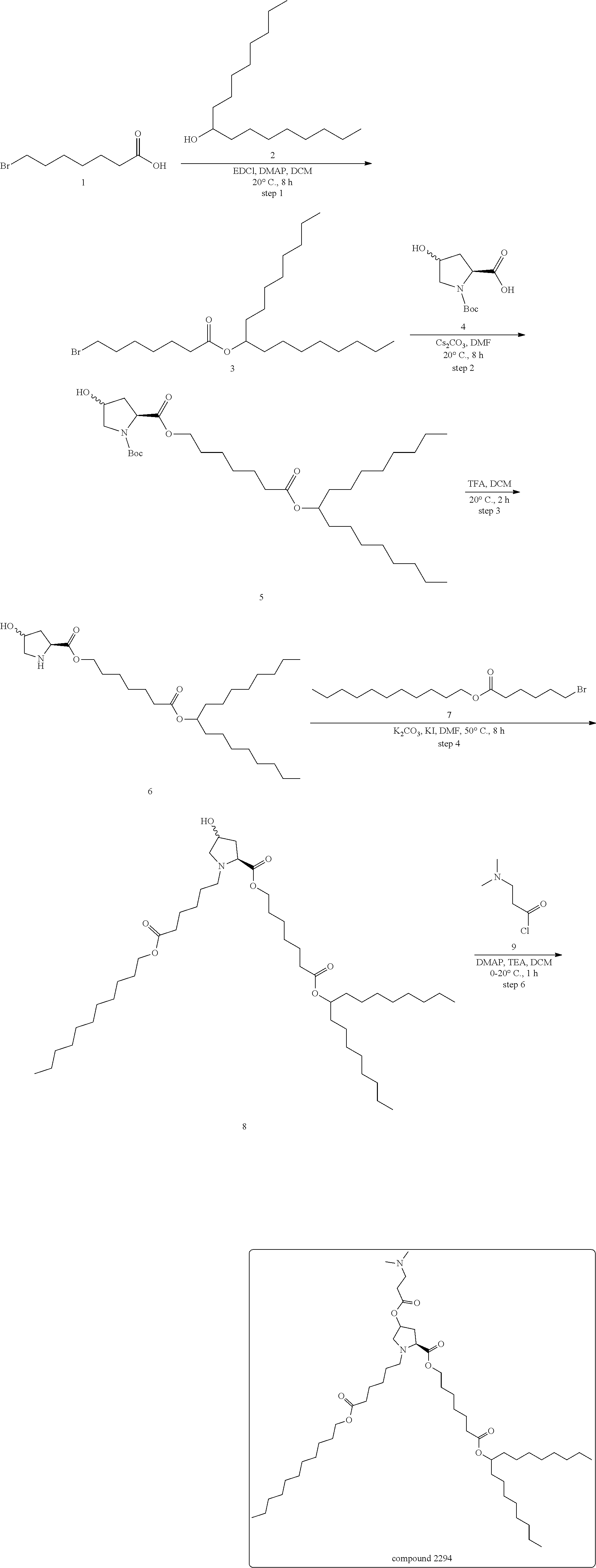

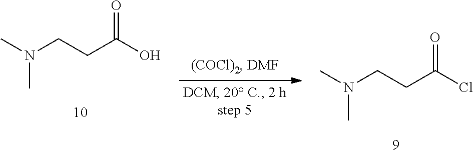

### Step 1

To a solution of heptadecan-9-ol (10 g, 38.99 mmol, 1 eq) and 7-bromoheptanoic acid (8.82 g, 42.17 mmol, 1.08 eq) in DCM (100 mL) was added DMAP (2.38 g, 19.50 mmol, 0.5 eq) and EDCI (8.97 g, 46.79 mmol, 1.2 eq). The mixture was stirred at 20° C. for 8 hours. The mixture was added into H₂O (200 mL), extracted with EtOAc (50 mL×3). The organic layer was washed with brine (50 mL×2), dried over Na₂SO₄, filtered, and concentrated under reduced pressure. The residue was purified by column chromatography (SiO₂, petroleum ether/ethyl acetate=1/0 to 5/1) to give 1-octylnonyl 7-bromoheptanoate (12 g, 26.81 mmol, 68.77% yield) as colorless oil.

### Step 2

To a solution of (2S)-1-tert-butoxycarbonyl-4-hydroxy-pyrrolidine-2-carboxylic acid (950 mg, 4.11 mmol, 1 eq) and 1-octylnonyl 7-bromoheptanoate (2.02 g, 4.52 mmol, 1.1 eq) in DMF (50 mL) was added Cs₂CO₃ (2.94 g, 9.04 mmol, 2.2 eq). The mixture was stirred at 20° C. for 8 hours. The mixture was added into H₂O (50 mL), extracted with EtOAc (20 mL×3). The organic layer was washed with brine (20 mL×2), dried over Na₂SO₄, filtered, and concentrated under reduced pressure. The residue was purified by column chromatography (SiO₂, petroleum ether/ethyl acetate=1/0 to 5/1) to give O1-tert-butyl O2-[7-(1-octylnonoxy)-7-oxo-heptyl](2S)-4-hydroxypyrrolidine-1,2-dicarboxylate (1.5 g, 2.51 mmol, 61.07% yield) as colorless oil.

### Step 3

A solution of O1-tert-butyl O2-[9-(1-octylnonoxy)-9-oxo-nonyl](2S)-4-hydroxypyrrolidine-1,2-dicarboxylate (1.5 g, 2.40 mmol, 1 eq) in DCM (30 mL) and TFA (6.93 g, 60.78 mmol, 4.50 mL, 25.36 eq) was stirred at 20° C. for 2 hours. The mixture was concentrated under reduced pressure to get residue. The residue was dissolved with EtOAc (20 mL), and the organic layer was washed with saturated NaHCO₃ (50 mL×4), brine (20 mL×2), dried over Na₂SO₄, filtered, and concentrated under reduced pressure to give [9-(1-octylnonoxy)-9-oxo-nonyl](2S)-4-hydroxypyrrolidine-2-carboxylate (1 g, 1.90 mmol, 79.36% yield) as colorless oil.

### Step 4

To a solution of [7-(1-octylnonoxy)-7-oxo-heptyl](2S)-4-hydroxypyrrolidine-2-carboxylate (0.5 g, 1.00 mmol, 1 eq), K₂CO₃ (416.49 mg, 3.01 mmol, 3 eq) and KI (83.38 mg, 502.26 μmol, 0.5 eq) in DMF (20 mL) was added undecyl 6-bromohexanoate (386.02 mg, 1.10 mmol, 1.1 eq). The mixture was stirred at 50° C. for 8 hours. The mixture was added into H₂O (20 mL), extracted with EtOAc (20 mL×3), and the organic layer was washed with brine (20 mL×2), dried over Na₂SO₄, filtered, and concentrated under reduced pressure. The residue was purified by column chromatography (SiO₂, petroleum ether/ethyl acetate=1/0 to 5/1) and purified by prep-HPLC (column: Phenomenex Luna C18 100×30 mmx 5 m; mobile phase: [water(HCl)-ACN]; B %: 55%-85%, 10 minutes) to get a solution. The solution was added saturated NaHCO₃ until the pH=˜7, and extracted with EtOAc (20 mL×3). The organic layer was washed with brine (20 mL×2), dried over Na₂SO₄, filtered, and, concentrated under reduced pressure to give [7-(1-octylnonoxy)-7-oxo-heptyl](2S)-4-hydroxy-1-(6-oxo-6-undecoxy-hexyl)pyrrolidine-2-carboxylate (1 g, 1.31 mmol, 64.96% yield) as colorless oil.

¹H NMR (400 MHz, CDCl₃), 4.84-4.91 (m, 1H), 4.25-4.55 (m, 1H), 4.04-4.20 (m, 4H), 3.05-3.75 (m, 2H), 1.91-2.85 (m, 9H), 1.60-1.70 (m, 8H), 1.45-1.55 (m, 6H), 1.20-1.40 (m, 46H), 0.89 (t, J=6.4 Hz, 9H). LCMS: (M+H⁺): 766.4 @13.405 minutes.

### Step 5

To a solution of 3-(dimethylamino)propanoic acid (300 mg, 1.95 mmol, 1 eq, HCl) in DCM (5 mL) was added (COCl)₂ (991.60 mg, 7.81 mmol, 683.86 μL, 4 eq) and DMF (14.27 mg, 195.30 mol, 15.03 μL, 0.1 eq), stirred at 20° C. for 2 hours. The mixture was concentrated under reduced pressure to give 3-(dimethylamino)propanoyl chloride (336 mg, 1.95 mmol, 99.99% yield, HCl) as a yellow solid.

### Step 6

To a solution of [7-(1-octylnonoxy)-7-oxo-heptyl](2S)-4-hydroxy-1-(6-oxo-6-undecoxy-hexyl) pyrrolidine-2-carboxylate (200 mg, 261.03 mol, 1 eq), DMAP (15.95 mg, 130.52 μmol, 0.5 eq) and TEA (132.07 mg, 1.31 mmol, 181.66 μL, 5 eq) in DCM (10 mL) was added 3-(dimethylamino) propanoyl chloride (112.00 mg, 650.96 mol, 2.49 eq, HCl) under N₂ at 0° C., and then the mixture was stirred at 20° C. for 1 hour. The mixture was added into saturated NaHCO₃ (20 mL), and extracted with EtOAc (10 mL×3). The organic layer was washed with brine (10 mL×2), dried over Na₂SO₄, filtered, and concentrated under reduced pressure. The residue was purified by column chromatography (SiO₂, petroleum ether/ethyl acetate=10/1 to 0/1) and purified by prep-HPLC (column: Phenomenex Luna C18 100×30 mm×5 m; mobile phase: [water(HCl)-ACN]; B %: 45%-75%, 10 minutes) to get a solution. The solution was added saturated NaHCO₃ until the pH=˜7, and extracted with EtOAc (20 mL×3). The organic layer was washed with brine (20 mL×2), dried over Na₂SO₄, filtered, and concentrated under reduced pressure to give [7-(1-octylnonoxy)-7-oxo-heptyl](2S)-4-[3-(dimethylamino)propanoyloxy]-1-(6-oxo-6-undecoxy-hexyl)pyrrolidine-2-carboxylate (70 mg, 80.90 μmol, 30.99% yield, 100% purity) as yellow oil.

¹H NMR (400 MHz, CDCl₃), 5.20-5.28 (m, 1H), 4.83-4.90 (m, 1H), 4.03-4.15 (m, 4H), 3.43-3.55 (m, 1H), 3.09-3.27 (m, 1H), 2.00-2.80 (m, 17H), 1.55-1.70 (m, 8H), 1.45-1.55 (m, 6H), 1.20-1.40 (m, 48H), 0.86-0.90 (m, 9H). LCMS: (M+H⁺): 865.4 @9.871/9.920 minutes.

### 8.6. Synthesis of Compound 2295

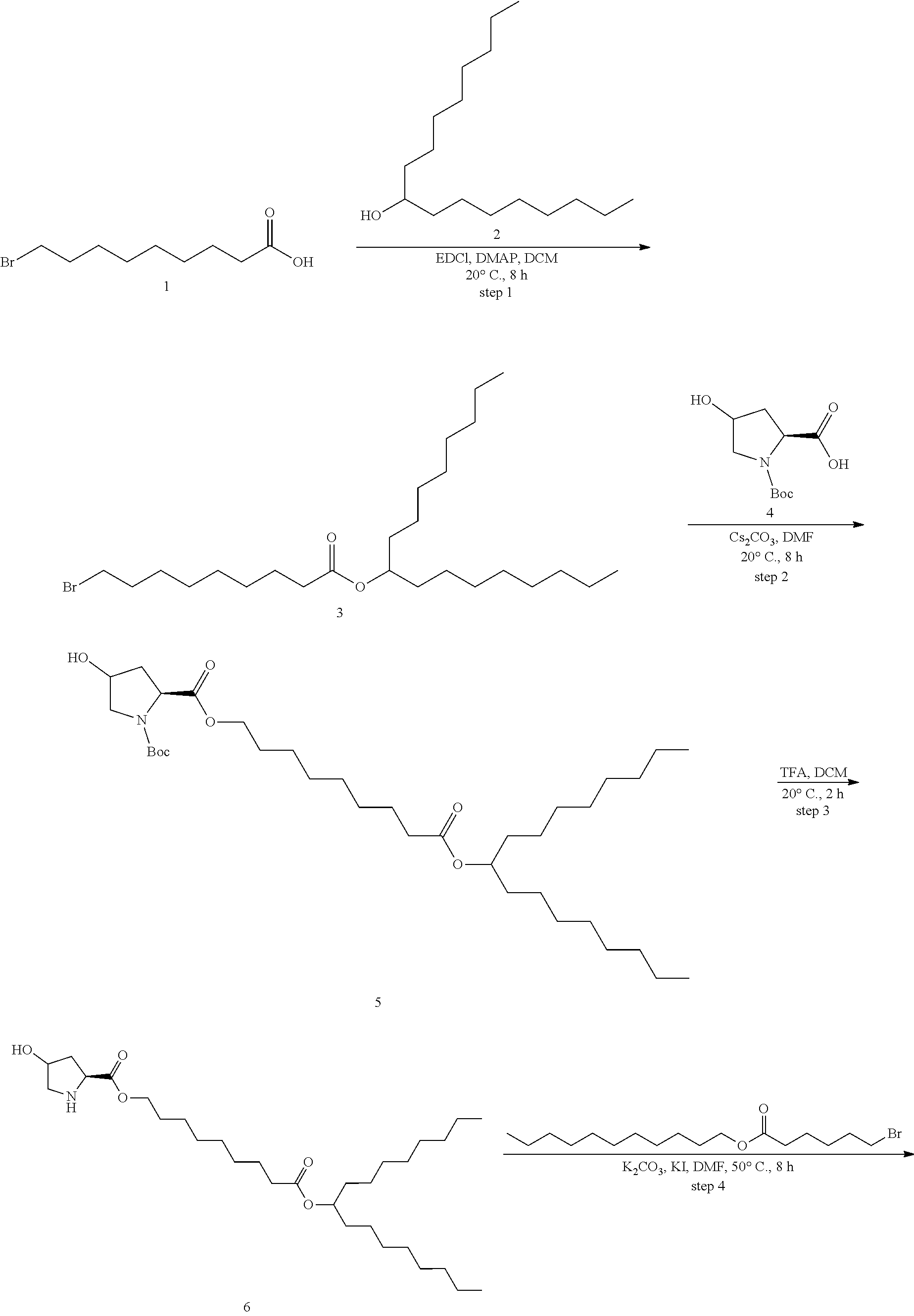

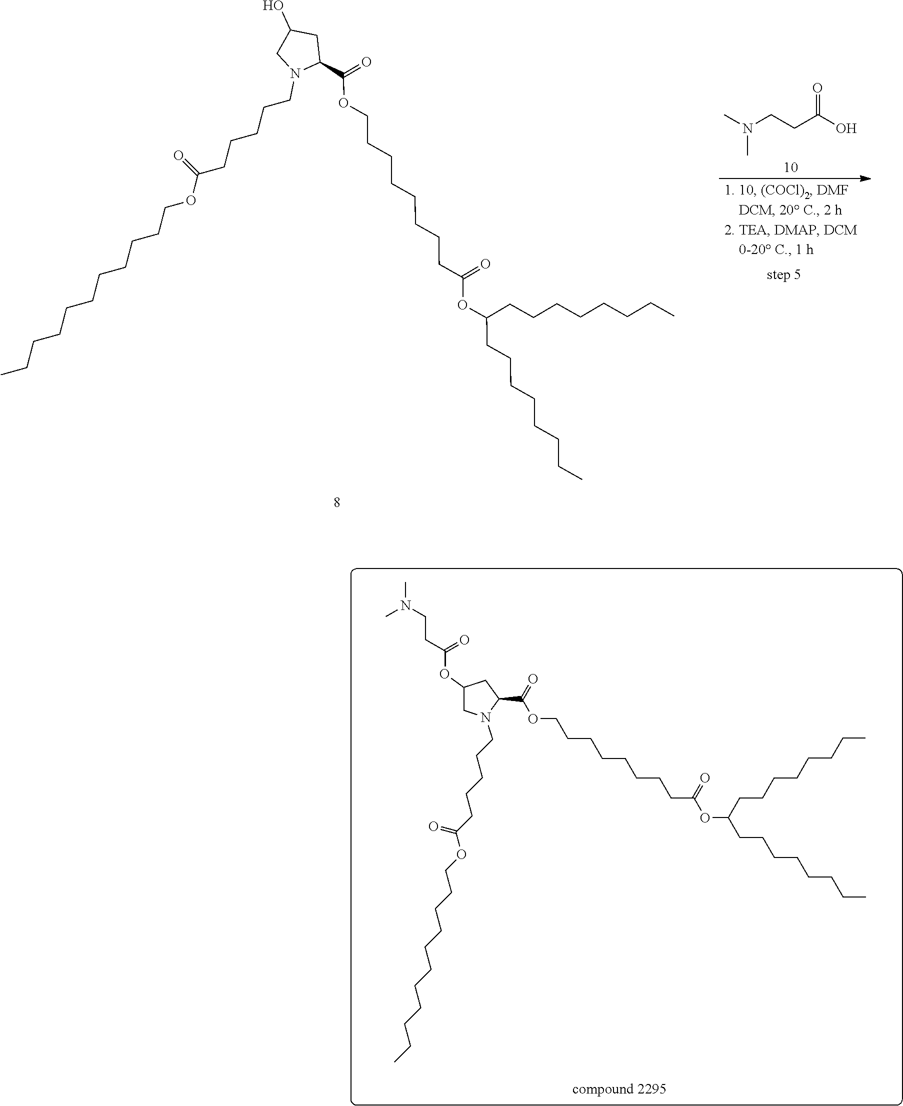

### Step 1

To a solution of heptadecan-9-ol (10 g, 38.99 mmol, 1 eq) and 9-bromononanoic acid (10 g, 42.17 mmol, 1.08 eq) in DCM (100 mL) was added DMAP (2.38 g, 19.50 mmol, 0.5 eq) and EDCI (8.97 g, 46.79 mmol, 1.2 eq). The mixture was stirred at 20° C. for 8 hours. The mixture was added into H₂O (200 mL), and extracted with EtOAc (100 mL×3). The organic layer was washed with brine (100 mL×2), dried over Na₂SO₄, filtered, and concentrated under reduced pressure. The residue was purified by column chromatography (SiO₂, petroleum ether/ethyl acetate=1/0 to 5/1) to get 1-octylnonyl 9-bromononanoate (15 g, 31.54 mmol, 80.89% yield) as colorless oil.

### Step 2

To a solution of (2S)-1-tert-butoxycarbonyl-4-hydroxy-pyrrolidine-2-carboxylic acid (5 g, 21.62 mmol, 1 eq) and 1-octylnonyl 9-bromononanoate (12.34 g, 25.95 mmol, 1.2 eq) in DMF (100 mL) was added Cs₂CO₃ (15.50 g, 47.57 mmol, 2.2 eq). The mixture was stirred at 20° C. for 8 hours. The mixture was added into H₂O (200 mL), and extracted with EtOAc (100 mL×3). The organic layer was washed with brine (100 mL×2), dried over Na₂SO₄, filtered, and concentrated under reduced pressure. The residue was purified by column chromatography (SiO₂, petroleum ether/ethyl acetate=1/0 to 5/1) to give O1-tert-butyl O2-[9-(1-octylnonoxy)-9-oxo-nonyl](2S)-4-hydroxypyrrolidine-1,2-dicarboxylate (10 g, 15.98 mmol, 73.89% yield) as colorless oil.

¹H NMR (400 MHz, CDCl₃), 4.85-4.89 (m, 1H), 4.05-4.55 (m, 4H), 3.40-3.80 (m, 2H), 2.25-2.40 (m, 3H), 2.05-2.15 (m, 1H), 1.60-1.75 (m, 4H), 1.40-1.60 (m, 14H), 1.20-1.35 (m, 32H), 0.88 (t, J=6.4 Hz, 6H).

### Step 3

A solution of O1-tert-butyl O2-[9-(1-octylnonoxy)-9-oxo-nonyl](2S)-4-hydroxypyrrolidine-1,2-dicarboxylate (10 g, 15.98 mmol, 1 eq) in DCM (60 mL) and TFA (57.75 g, 506.48 mmol, 37.50 mL, 31.70 eq) was stirred at 20° C. for 2 hours. The mixture was concentrated under reduced pressure to give a residue. The residue was dissolved with EtOAc (100 mL), washed with saturated NaHCO₃ (200 mL×2) and brine (200 mL×2), dried over Na₂SO₄, filtered, and concentrated under reduced pressure to give [9-(1-octylnonoxy)-9-oxo-nonyl](2S)-4-hydroxypyrrolidine-2-carboxylate (7 g, crude) as yellow oil.

### Step 4

To a solution of [9-(1-octylnonoxy)-9-oxo-nonyl](2S)-4-hydroxypyrrolidine-2-carboxylate (2 g, 3.80 mmol, 1 eq), K₂CO₃ (1.58 g, 11.41 mmol, 3 eq) and KI (315.71 mg, 1.90 mmol, 0.5 eq) in DMF (100 mL) was added undecyl 6-bromohexanoate (1.59 g, 4.56 mmol, 1.2 eq). The mixture was stirred at 50° C. for 8 hours. The mixture was added into H₂O (20 mL), and extracted with EtOAc (20 mL×3). The organic layer was washed with brine (20 mL×2), dried over Na₂SO₄, filtered, and concentrated under reduced pressure. The residue was purified by column chromatography (SiO₂, petroleum ether/ethyl acetate=1/0 to 5/1) and purified by prep-HPLC (column: Phenomenex Luna C18 100×30 mm×5 m; mobile phase: [water(HCl)-ACN]; B %: 55%-80%, 10 minutes) to give [9-(1-octylnonoxy)-9-oxo-nonyl](2S)-4-hydroxy-1-(6-oxo-6-undecoxy-hexyl)pyrrolidine-2-carboxylate (2 g, 2.52 mmol, 66.20% yield) as yellow oil.

¹H NMR (400 MHz, CDCl₃), 4.85-4.90 (m, 1H), 4.20-4.55 (m, 1H), 4.00-4.15 (m, 4H), 3.05-3.60 (m, 2H), 1.90-2.80 (m, 9H), 1.55-1.75 (m, 8H), 1.45-1.55 (m, 6H), 1.20-1.40 (m, 50H), 0.88 (t, J=6.4 Hz, 9H).

### Step 5

To a solution of 3-(dimethylamino)propanoic acid (100 mg, 651.01 mol, 1 eq, HCl) in DCM (5 mL) was added (COCl)₂ (330.53 mg, 2.60 mmol, 227.95 μL, 4 eq) and DMF (4.76 mg, 65.10 mol, 5.01 μL, 0.1 eq). The mixture was stirred at 20° C. for 2 hours. The mixture was concentrated under reduced pressure to give 3-(dimethylamino)propanoyl chloride (112 mg, crude, HCl) as a yellow solid. Then 3-(dimethylamino)propanoyl chloride (112 mg, 650.96 μmol, 2.59 eq, HCl) was added to a solution of [9-(1-octylnonoxy)-9-oxo-nonyl](2S)-4-hydroxy-1-(6-oxo-6-undecoxy-hexyl)pyrrolidine-2-carboxylate (200 mg, 251.81 mol, 1 eq), DMAP (15.38 mg, 125.91 mol, 0.5 eq), and TEA (127.41 mg, 1.26 mmol, 175.25 μL, 5 eq) in DCM (5 mL) under N₂ at 0° C., and then the mixture was stirred at 20° C. for 1 hour. The mixture was added into saturated NaHCO₃ (20 mL), and extracted with EtOAc (10 mL×3). The organic layer was washed with brine (10 mL×2), dried over Na₂SO₄, filtered, and concentrated under reduced pressure. The residue was purified by column chromatography (SiO₂, petroleum ether/ethyl acetate=10/1 to 0/1) and further purified by prep-TLC (SiO₂, ethyl acetate/MeOH=5:1, added 3% NH₃·H₂O) to give [9-(1-octylnonoxy)-9-oxo-nonyl](2S)-4-[3-(dimethylamino)propanoyloxy]-1-(6-oxo-6-undecoxy-hexyl)pyrrolidine-2-carboxylate (100 mg, 108.58 mol, 43.12% yield, 97% purity) as yellow oil.

¹H NMR (400 MHz, CDCl₃), 5.20-5.29 (m, 1H), 4.85-4.89 (m, 1H), 4.03-4.18 (m, 4H), 3.44-3.55 (m, 1H), 3.09-3.26 (m, 1H), 2.05-2.80 (m, 19H), 1.60-1.65 (m, 8H), 1.45-1.55 (m, 6H), 1.18-1.40 (m, 50H), 0.86-0.91 (m, 9H). LCMS: (M+H⁺): 893.5 @10.397/10.417 minutes.

### 8.7. Synthesis of Compound 2296

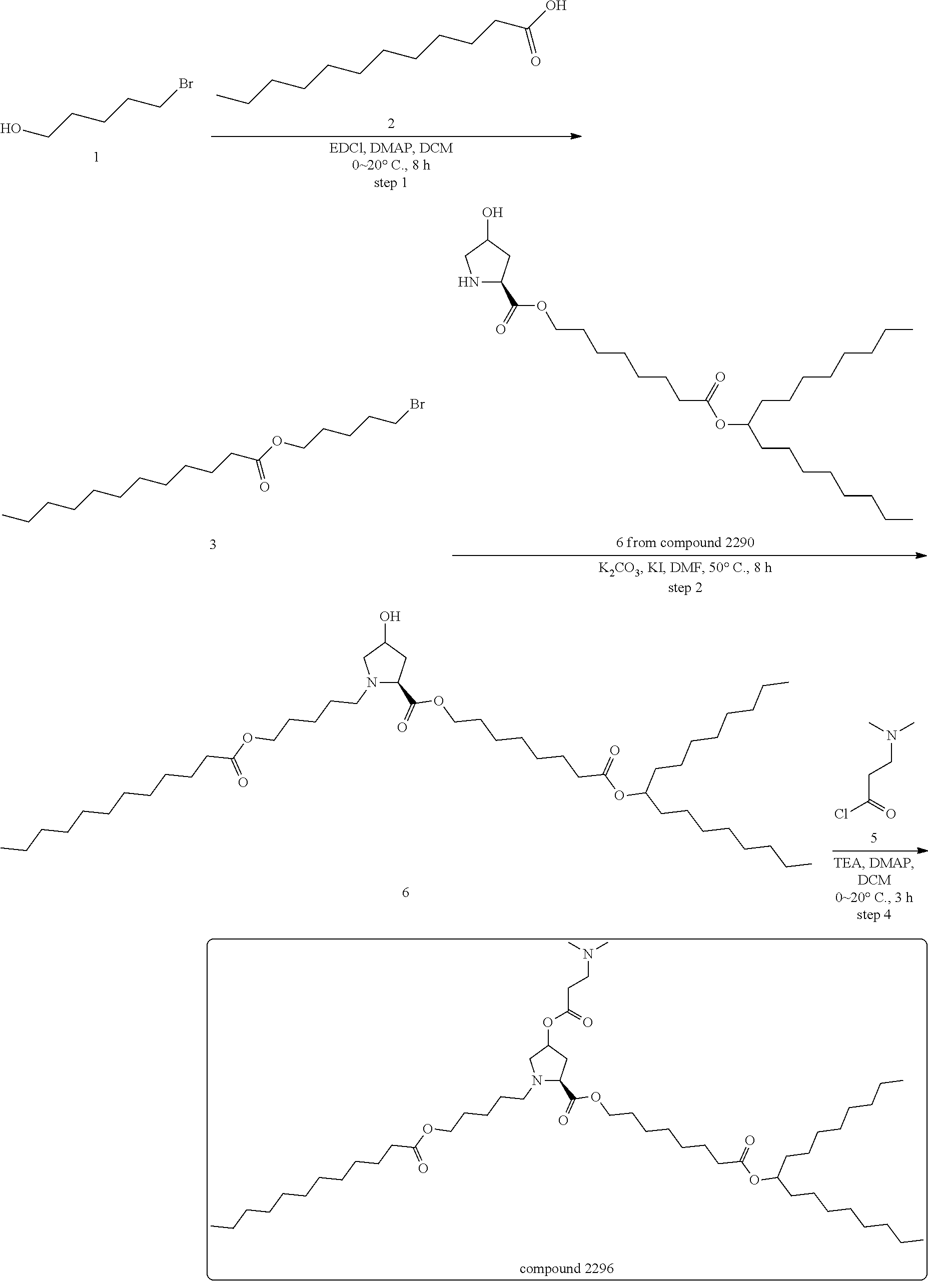

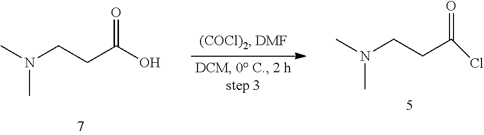

### Step 1

To a solution of 5-bromopentan-1-ol (10 g, 59.86 mmol, 1 eq) and dodecanoic acid (12.59 g, 62.86 mmol, 1.05 eq) in DCM (100 mL) was added EDCI (22.95 g, 119.73 mmol, 2 eq) and DMAP (3.66 g, 29.93 mmol, 0.5 eq) at 0° C. The mixture was stirred at 20° C. for 8 hours. The reaction mixture was quenched by addition of mL H₂O 200 at 0° C., and then extracted with 300 mL EtOAc (100 mL×3). The combined organic layers were washed with 300 mL saturated brine (100 mL×3), dried over Na₂SO₄, filtered, and concentrated under reduced pressure to give a residue. The residue was purified by column chromatography (SiO₂, petroleum ether/ethyl acetate=100/1 to 5/1) to give 5-bromopentyl dodecanoate (15 g, 42.94 mmol, 71.72% yield) as a white solid.

### Step 2

To a solution of [8-(1-octylnonoxy)-8-oxo-octyl](2S)-4-hydroxypyrrolidine-2-carboxylate (800 mg, 1.56 mmol, 1 eq) in DMF (10 mL) was added K₂CO₃ (648.13 mg, 4.69 mmol, 3 eq) and KI (129.75 mg, 781.59 mol, 0.5 eq). Then 5-bromopentyl dodecanoate (600.70 mg, 1.72 mmol, 1.1 eq) was added to the mixture. The mixture was stirred at 50° C. for 8 hours. The reaction mixture was quenched by addition of 20 mL H₂O at 0° C., and then extracted with 60 mL EtOAc (20 mL×3). The combined organic layers were washed with 60 mL saturated brine (20 mL×3), dried over Na₂SO₄, filtered, and concentrated under reduced pressure to give a residue. The residue was purified by column chromatography (SiO₂, petroleum ether/ethyl acetate=10/1 to 0/1) to give [8-(1-octylnonoxy)-8-oxo-octyl](2S)-1-(5-dodecanoyloxypentyl)-4-hydroxy-pyrrolidine-2-carboxylate (820 mg, 1.05 mmol, 67.23% yield) as yellow oil.

### Step 3

To a solution of 3-(dimethylamino)propanoic acid (400 mg, 2.60 mmol, 1 eq, HCl) in DCM (10 mL) was added DMF (9.52 mg, 130.20 mol, 10.02 μL, 0.05 eq) and (COCl)₂ (396.63 mg, 3.12 mmol, 273.54 μL, 1.2 eq) at 0° C. The mixture was stirred at 0° C. for 2 hours. The mixture was concentrated under reduced pressure to give 3-(dimethylamino)propanoyl chloride (450 mg, crude, HCl) as a white solid.

### Step 4

To a solution of [8-(1-octylnonoxy)-8-oxo-octyl](2S)-1-(5-dodecanoyloxypentyl)-4-hydroxy-pyrrolidine-2-carboxylate (400 mg, 512.68 mol, 1 eq) in DCM (10 mL) was added TEA (518.78 mg, 5.13 mmol, 713.59 μL, 10 eq) and 3-(dimethylamino)propanoyl chloride (352.83 mg, 2.05 mmol, 4 eq, HCl) at 0° C. The mixture was stirred at 20° C. for 3 hours. The reaction mixture was quenched by addition of 10 mL H₂O at 0° C., and then extracted with 30 mL EtOAc (10 mL×3). The combined organic layers were washed with 30 mL saturated brine (10 mL×3), dried over Na₂SO₄, filtered, and concentrated under reduced pressure to give a residue. The residue was purified by prep-HPLC (column: Phenomenex Luna C18 100×30 mm×5 m; mobile phase: [water(HCl)-ACN]; B %: 45%-75%, 10 minutes) to give a solution. The solution was adjusted to pH=8 with saturated NaHCO₃, and extracted with 30 mL EtOAc (10 mL×3). The combined organic layers were dried over Na₂SO₄, filtered, and concentrated under reduced pressure to give [8-(1-octylnonoxy)-8-oxo-octyl](2S)-4-[3-(dimethylamino)propanoyloxy]-1-(5-dodecanoyloxypentyl)pyrrolidine-2-carboxylate (108 mg, 51.58 mol, 10.06% yield, 42% purity) as colorless oil.

¹H NMR (400 MHz, CDCl₃), 5.21-5.28 (m, 1H), 4.84-4.88 (m, 1H), 4.10-4.13 (m, 2H), 4.06 (t, J=6.8 Hz, 2H), 3.12-3.55 (m, 2H), 1.97-2.35 (m, 7H), 2.40-2.57 (m, 12H), 1.60-1.65 (m, 6H), 1.50-1.52 (m, 6H), 1.26-1.38 (m, 50H), 0.89 (t, J=6.8 Hz, 9H).

LCMS: (M+H⁺): 879.4 @10.062 minutes.

### 8.8. Synthesis of Compound 2297

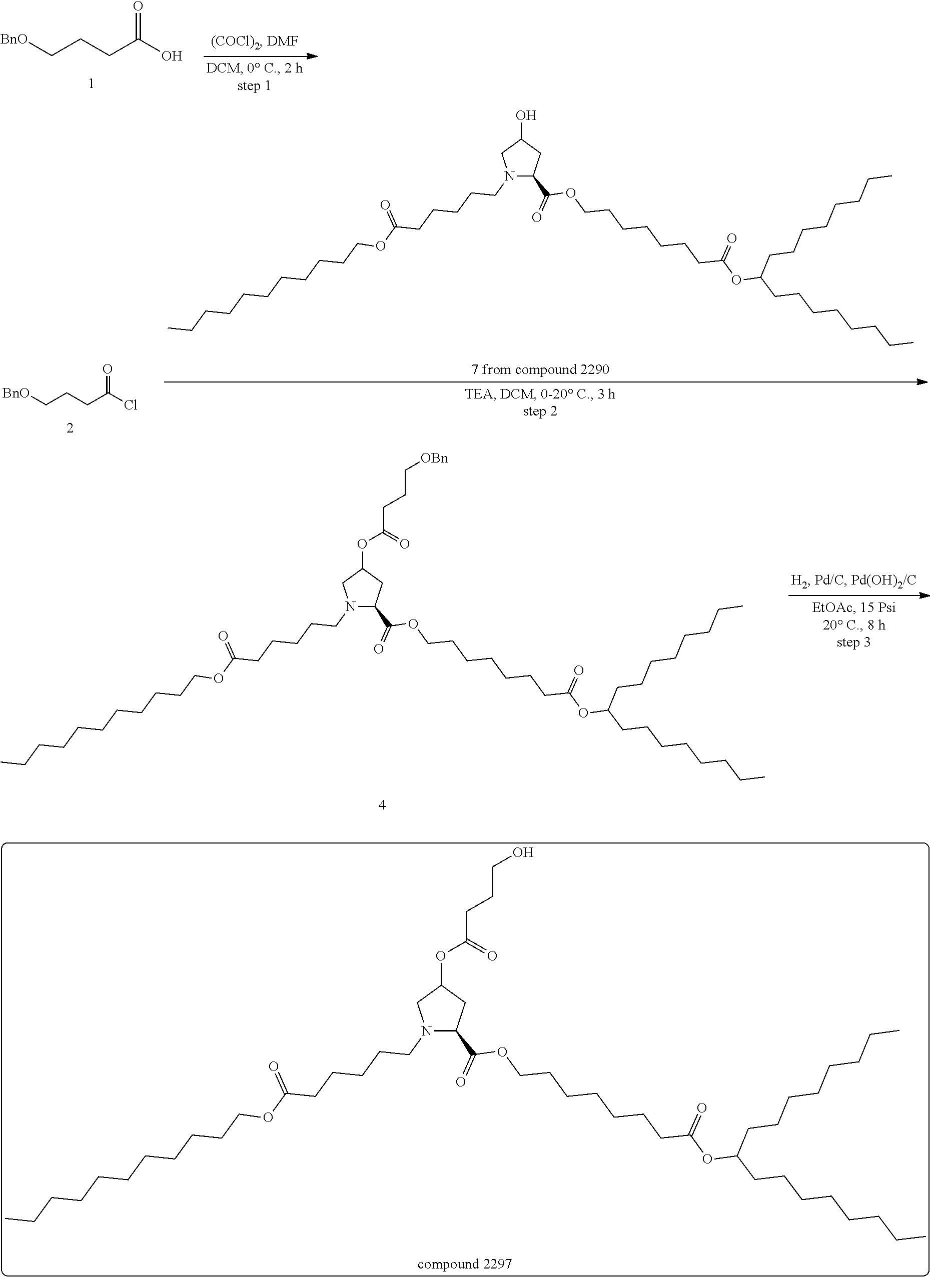

### Step 1

To a solution of 4-benzyloxybutanoic acid (2 g, 10.30 mmol, 1.82 mL, 1 eq) in DCM (20 mL) was added DMF (15.05 mg, 205.95 mol, 15.85 μL, 0.02 eq) and (COCl)₂ (1.57 g, 12.36 mmol, 1.08 mL, 1.2 eq) at 0° C. The mixture was stirred at 0° C. for 2 hours. The mixture was concentrated under reduced pressure to give 4-benzyloxybutanoyl chloride (2.2 g, crude) as a white solid.

### Step 2

To a solution of [8-(1-octylnonoxy)-8-oxo-octyl](2S)-4-hydroxy-1-(6-oxo-6-undecoxy-hexyl)pyrrolidine-2-carboxylate (1.5 g, 1.92 mmol, 1 eq) in DCM (20 mL) was added TEA (1.95 g, 19.23 mmol, 2.68 mL, 10 eq) and 4-benzyloxybutanoyl chloride (2.04 g, 9.61 mmol, 5 eq) at 0° C. The mixture was stirred at 20° C. for 3 hours. The reaction mixture was quenched by addition of 20 mL H₂O at 0° C., and then extracted with 60 mL EtOAc (20 mL×3). The combined organic layers were washed with 60 mL brine (20 mL×3), dried over Na₂SO₄, filtered, and concentrated under reduced pressure to give a residue. The residue was purified by column chromatography (SiO₂, petroleum ether/ethyl acetate=20/1 to 1/1) to give [8-(1-octylnonoxy)-8-oxo-octyl](2S)-4-(4-benzyloxybutanoyloxy)-1-(6-oxo-6-undecoxy-hexyl)pyrrolidine-2-carboxylate (1.3 g, 1.36 mmol, 70.70% yield) as yellow oil.

### Step 3

To a solution of [8-(1-octylnonoxy)-8-oxo-octyl](2S)-4-(4-benzyloxybutanoyloxy)-1-(6-oxo-6-undecoxy-hexyl)pyrrolidine-2-carboxylate (600 mg, 627.34 mol, 1 eq) in EtOAc (10 mL) was added Pd/C (0.3 g, 10% purity) and Pd(OH)₂/C (0.3 g, 427.25 mol, 20% purity, 6.81e-1 eq). The mixture was stirred at 20° C. for 8 hours under H₂ atmosphere (15 psi). The mixture was filtered and the filtrate was concentrated under reduced pressure to give a residue. The residue was purified by prep-HPLC (column: Phenomenex Luna C18 100×30 mm×5 m; mobile phase: [water(HCl)-ACN]; B %: 50%-80%, 10 minutes). Then the mixture was adjusted to pH=8 with saturated NaHCO₃, and extracted with 30 mL EtOAc (10 mL×3). The combined organic layers were dried over Na₂SO₄, filtered, and concentrated under reduced pressure to give [8-(1-octylnonoxy)-8-oxo-octyl](2S)-4-(4-hydroxybutanoyloxy)-1-(6-oxo-6-undecoxy-hexyl)pyrrolidine-2-carboxylate (191 mg, 220.48 μmol, 35.15% yield) as colorless oil.

1H NMR (400 MHz, CDCl₃), 5.15-5.28 (m, 1H), 4.85-4.88 (m, 1H), 4.03-4.12 (m, 4H), 3.27-3.73 (m, 4H), 2.26-2.72 (m, 11H), 1.86-1.91 (m, 2H), 1.60-1.70 (m, 8H), 1.45-1.55 (m, 6H), 1.26-1.34 (m, 48H), 0.89 (t, J=5.2 Hz, 9H). (M+H⁺): 866.8.

LCMS: (M+H⁺): 866.8 @13.884 minutes.

### 8.9. Synthesis of Compound 2298

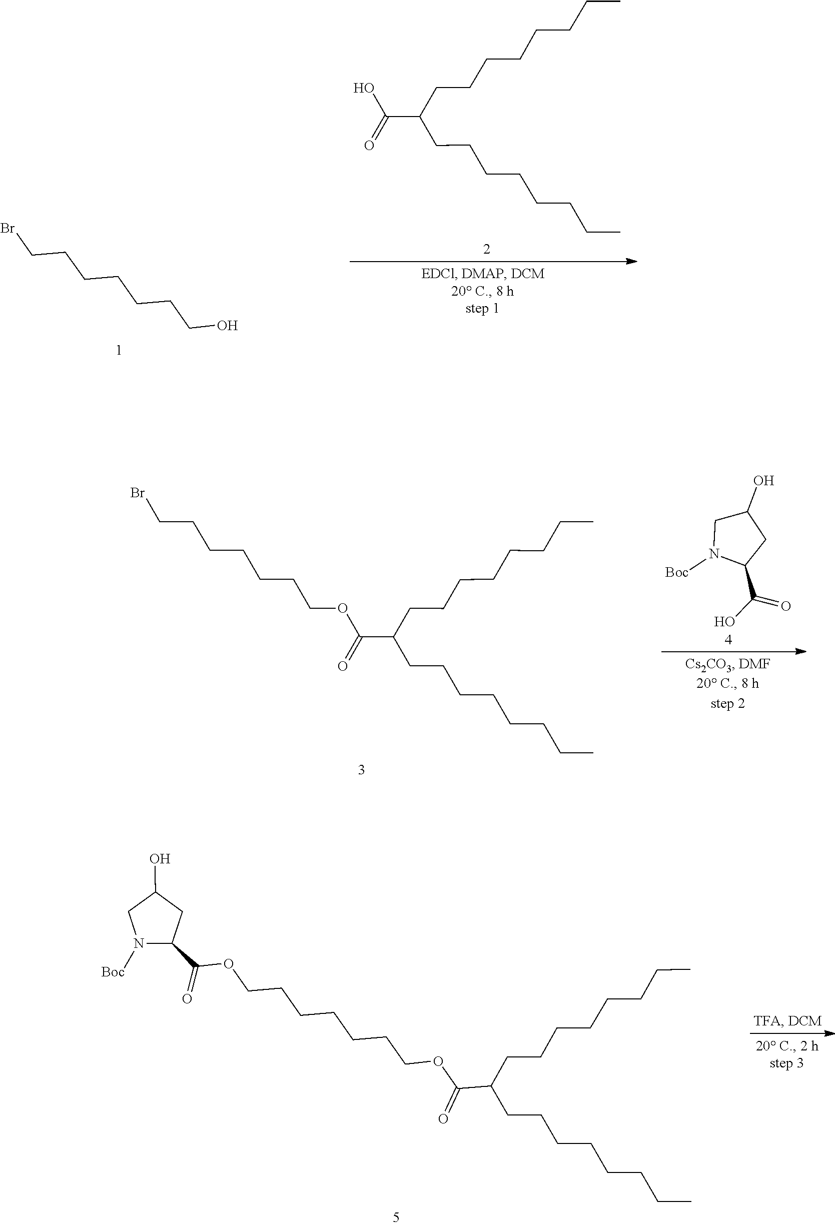 [SMILES: OCCCCCCCBr ; CCCCCCCCC(CCCCCCCC)C(=O)O ; CCCCCCCCC(CCCCCCCC)C(=O)OCCCCCCCBr ; CC(C)(C)OC(=O)N1CC(O)C[C@H]1C(=O)O ; CCCCCCCCC(CCCCCCCC)C(=O)OCCCCCCCOC(=O)[C@@H]1CC(O)CN1C(=O)OC(C)(C)C]

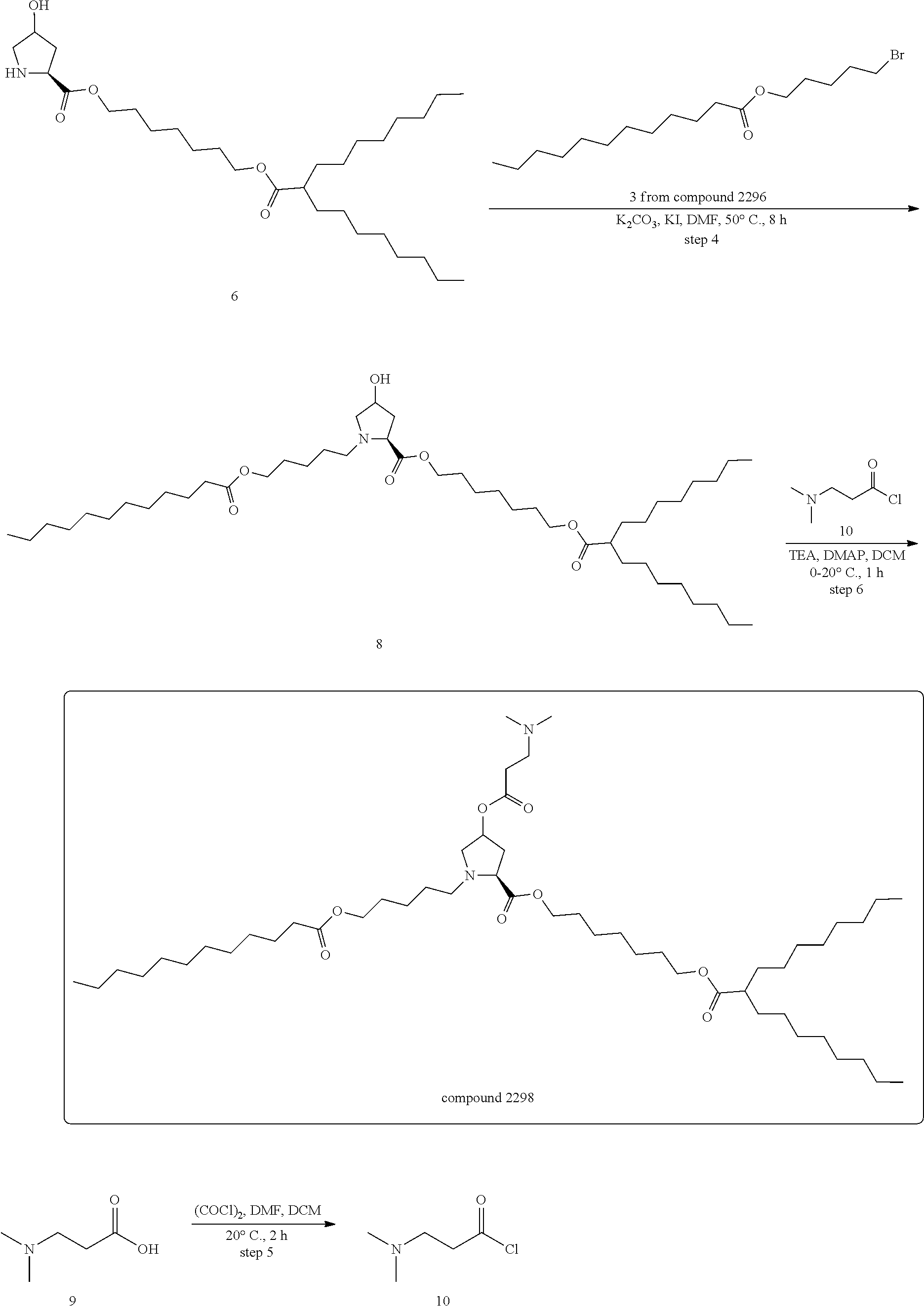

### Step 1

To a solution of 7-bromoheptan-1-ol (3.60 g, 18.46 mmol, 1.05 eq) and 2-octyldecanoic acid (5 g, 17.58 mmol, 1 eq) in DCM (100 mL) was added DMAP (1.07 g, 8.78 mmol, 0.5 eq) and EDCI (4.04 g, 21.10 mmol, 1.2 eq). The mixture was stirred at 20° C. for 8 hours. The mixture was added into H₂O (200 mL), and extracted with EtOAc (200 mL×3). The organic layer was washed with brine (200 mL×2), dried over Na₂SO₄, filtered, and concentrated under reduced pressure. The residue was purified by column chromatography (SiO₂, petroleum ether/ethyl acetate=1/0 to 5/1) to give 7-bromoheptyl 2-octyldecanoate (7 g, 15.17 mmol, crude) as colorless oil.

### Step 2

To a solution of (2S)-1-tert-butoxycarbonyl-4-hydroxy-pyrrolidine-2-carboxylic acid (3 g, 12.96 mmol, 1 eq) and 7-bromoheptyl 2-octyldecanoate (6.99 g, 15.18 mmol, 1.17 eq) in DMF (100 mL) was added Cs₂CO₃ (9.30 g, 28.53 mmol, 2.2 eq). The mixture was stirred at 20° C. for 8 hours. The mixture was added into H₂O (20 mL), and extracted with EtOAc (20 mL×3). The organic layer was washed with brine (20 mL×2), dried over Na₂SO₄, filtered, and concentrated under reduced pressure. The residue was purified by column chromatography (SiO₂, petroleum ether/ethyl acetate=1/0 to 5/1) to give O1-tert-butyl O2-[7-(2-octyldecanoyloxy)heptyl](2S)-4-hydroxypyrrolidine-1,2-dicarboxylate (5 g, 8.17 mmol, 62.99% yield) as colorless oil.

### Step 3

A solution of O1-tert-butyl O2-[7-(2-octyldecanoyloxy)heptyl](2S)-4-hydroxypyrrolidine-1,2-dicarboxylate (5 g, 8.18 mmol, 1 eq) in DCM (30 mL) and TFA (23.10 g, 101.30 mmol, 15.02 mL, 24.79 eq) was stirred at 20° C. for 2 hours. The mixture was concentrated under reduced pressure to get a residue, and the residue was dissolved with EtOAc (20 mL). The organic layer was washed with saturated NaHCO₃ (50 mL×4) and brine (20 mL×2), dried over Na₂SO₄, filtered, and concentrated under reduced pressure to give 7-(2-octyldecanoyloxy)heptyl (2S)-4-hydroxypyrrolidine-2-carboxylate (4 g, crude) as colorless oil.

### Step 4

To a solution of 7-(2-octyldecanoyloxy)heptyl (2S)-4-hydroxypyrrolidine-2-carboxylate (2 g, 3.91 mmol, 1 eq), K₂CO₃ (1.62 g, 11.72 mmol, 3 eq) and KI (324.36 mg, 1.95 mmol, 0.5 eq) in DMF (20 mL) was added 5-bromopentyl dodecanoate (1.50 g, 4.30 mmol, 1.1 eq). The mixture was stirred at 50° C. for 8 hours. The mixture was added into H₂O (20 mL), and extracted with EtOAc (20 mL×3). The organic layer was washed with brine (20 mL×2), dried over Na₂SO₄, filtered, and concentrated under reduced pressure. The residue was purified by column chromatography (SiO₂, petroleum ether/ethyl acetate=1/0 to 5/1) and purified by prep-HPLC (column: Phenomenex Luna C18 100×30 mm×5 m; mobile phase: [water(HCl)-ACN]; B %: 50%-80%, 10 minutes) to give a solution. The solution was added saturated NaHCO₃ until the solution has a pH=˜7, and extracted with EtOAc (20 mL×3). The organic layer was washed with brine (20 mL×2), dried over Na₂SO₄, filtered, and concentrated under reduced pressure to give 7-(2-octyldecanoyloxy)heptyl (2S)-1-(5-dodecanoyloxypentyl)-4-hydroxy-pyrrolidine-2-carboxylate (2 g, 2.56 mmol, 65.59% yield) as colorless oil.

¹H NMR (400 MHz, CDCl₃), 4.25-4.55 (m, 1H), 3.95-4.25 (m, 6H), 3.05-3.75 (m, 2H), 1.80-2.85 (m, 8H), 1.60-1.70 (m, 8H), 1.40-1.55 (m, 6H), 1.23-1.40 (m, 48H), 0.89 (t, J=6.4 Hz, 9H).

LCMS: (M+H⁺): 780.4 @13.242&13.267 minutes.

### Step 5

To a solution of 3-(dimethylamino)propanoic acid (300 mg, 1.95 mmol, 1 eq, HCl) in DCM (5 mL) was added (COCl)₂ (991.60 mg, 7.81 mmol, 683.86 μL, 4 eq) and DMF (14.27 mg, 195.30 mol, 15.03 μL, 0.1 eq). The mixture was stirred at 20° C. for 2 hours. The mixture was concentrated under reduced pressure to give 3-(dimethylamino)propanoyl chloride (336 mg, 1.95 mmol, 99.99% yield, HCl) as a yellow solid.

### Step 6

To a solution of 7-(2-octyldecanoyloxy)heptyl (2S)-1-(5-dodecanoyloxypentyl)-4-hydroxy-pyrrolidine-2-carboxylate (350 mg, 448.60 mol, 1 eq), DMAP (27.40 mg, 224.30 mol, 0.5 eq) and TEA (226.97 mg, 2.24 mmol, 312.20 μL, 5 eq) in DCM (10 mL) was added 3-(dimethylamino)propanoyl chloride (294.00 mg, 1.71 mmol, 3.81 eq, HCl) under N₂ at 0° C., and then the mixture was stirred at 20° C. for 1 hour. The mixture was added into saturated NaHCO₃ (20 mL), and extracted with EtOAc (10 mL×3). The organic layer was washed with brine (10 mL×2), dried over Na₂SO₄, filtered, and concentrated under reduced pressure. The residue was purified by column chromatography (SiO₂, petroleum ether/ethyl acetate=10/1 to 0/1) and purified by prep-HPLC (column: Phenomenex Luna C18 100×30 mm×5 m; mobile phase: [water(HCl)-ACN]; B %: 50%-80%, 10 minutes) to give a solution. The solution was added saturated NaHCO₃ until the solution has pH=˜7, and extracted with EtOAc (20 mL×3). The organic layer was washed with brine (20 mL×2), dried over Na₂SO₄, filtered, and concentrated under reduced pressure to give 7-(2-octyldecanoyloxy)heptyl (2S)-4-[3-(dimethylamino)propanoyloxy]-1-(5-dodecanoyloxypentyl) pyrrolidine-2-carboxylate (132 mg, 148.61 mol, 45.40% yield, 99% purity) as yellow oil.

¹H NMR (400 MHz, CDCl₃), 5.20-5.30 (m, 1H), 4.03-4.17 (m, 6H), 3.25-3.55 (m, 1H), 3.09-3.30 (m, 1H), 2.00-2.80 (m, 18H), 1.57-1.70 (m, 8H), 1.45-1.55 (m, 6H), 1.15-1.40 (m, 48H), 0.89 (t, J=6.8 Hz, 9H). LCMS: (M+H⁺): 879.7 @9.868 minutes.

### 8.10. Synthesis of Compound 2304

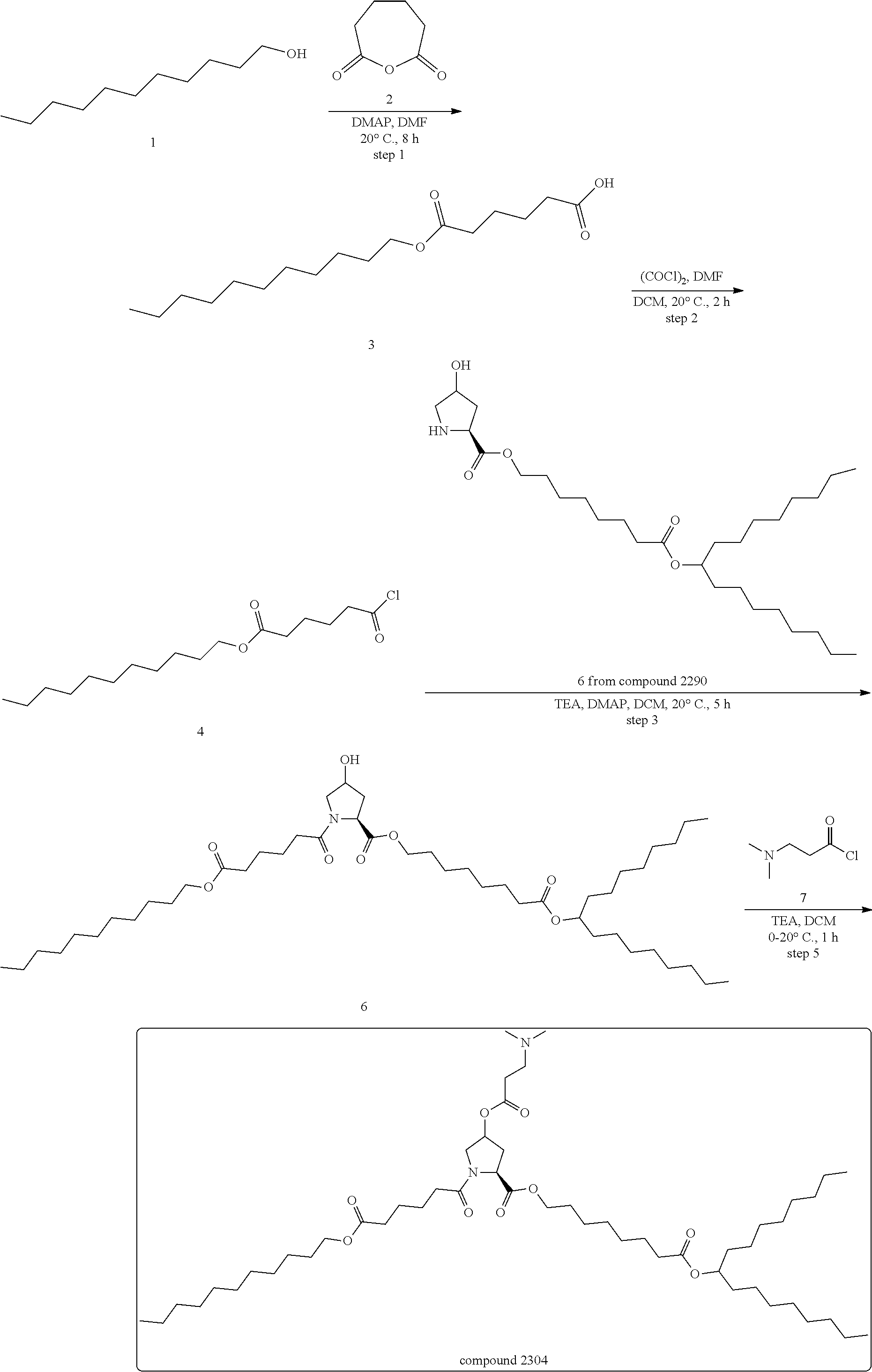

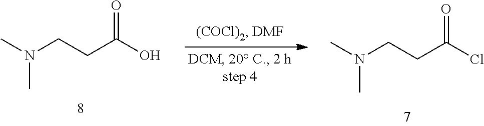

### Step 1

To a solution of undecan-1-ol (5 g, 29.02 mmol, 1 eq) and DMAP (3.55 g, 29.02 mmol, 1 eq) in DMF (10 mL) was added oxepane-2,7-dione (4.46 g, 34.82 mmol, 1.2 eq). The mixture was stirred at 20° C. for 8 hours. The mixture was added into H₂O (20 mL), and extracted with EtOAc (20 mL×3). The organic layer was washed with brine (20 mL×2), dried over Na₂SO₄, filtered, and concentrated under reduced pressure. The residue was purified by column chromatography (SiO₂, petroleum ether/ethyl acetate=1/0 to 5/1) to give 6-oxo-6-undecoxy-hexanoic acid (4 g, 13.31 mmol, 45.88% yield) as a white solid.

¹H NMR (400 MHz, CDCl₃), 4.07 (t, J=6.8 Hz, 2H), 2.33-2.41 (m, 4H), 1.60-1.71 (m, 6H), 1.19-1.41 (m, 17H), 0.89 (m, J=7.2 Hz, 3H).

### Step 2

To a solution of 6-oxo-6-undecoxy-hexanoic acid (2 g, 6.66 mmol, 1 eq) in DCM (10 mL) was added (COCl)₂ (2.53 g, 19.97 mmol, 1.75 mL, 3 eq) and DMF (4.87 mg, 66.57 mol, 5.12 μL, 0.01 eq). The mixture was stirred at 20° C. for 2 hours. The mixture was concentrated under reduced pressure to give undecyl 6-chloro-6-oxo-hexanoate (2.12 g, crude) as a white solid.

### Step 3

To a solution of [8-(1-octylnonoxy)-8-oxo-octyl](2S)-4-hydroxypyrrolidine-2-carboxylate (3 g, 5.86 mmol, 1 eq), TEA (1.78 g, 17.59 mmol, 2.45 mL, 3 eq) and DMAP (71.61 mg, 586.20 μmol, 0.1 eq) in DCM (5 mL) was added undecyl 6-chloro-6-oxo-hexanoate (1.91 g, 5.99 mmol, 1.02 eq). The mixture was stirred at 20° C. for 5 hours. The mixture was added into H₂O (20 mL), and extracted with EtOAc (20 mL×3). The organic layer was washed with brine (20 mL×2), dried over Na₂SO₄, filtered, and concentrated under reduced pressure. The residue was purified by column chromatography (SiO₂, petroleum ether/ethyl acetate=1/0 to 5/1) to give [8-(1-octylnonoxy)-8-oxo-octyl](2S)-4-hydroxy-1-(6-oxo-6-undecoxy-hexanoyl)pyrrolidine-2-carboxylate (2 g, 2.52 mmol, 42.96% yield) as a white solid.

¹H NMR (400 MHz, CDCl₃), 4.80-4.90 (m, 1H), 4.40-4.65 (m, 2H), 4.05-4.25 (m, 4H), 3.49-3.88 (m, 2H), 2.20-2.40 (m, 8H), 1.60-1.75 (m, 10H), 1.40-1.53 (m, 4H), 1.23-1.38 (m, 46H), 0.89 (t, J=6.8 Hz, 9H).

### Step 4

To a solution of 3-(dimethylamino)propanoic acid (400 mg, 2.60 mmol, 1 eq, HCl) in DCM (5 mL) was added (COCl)₂ (1.32 g, 10.42 mmol, 911.81 μL, 4 eq) and DMF (19.03 mg, 260.40 mol, 20.03 μL, 0.1 eq). The mixture was stirred at 20° C. for 2 hours. The mixture was concentrated under reduced pressure to give 3-(dimethylamino)propanoyl chloride (448 mg, crude, HCl) as a yellow solid.

### Step 5

To a solution of [8-(1-octylnonoxy)-8-oxo-octyl](2S)-4-hydroxy-1-(6-oxo-6-undecoxy-hexanoyl)pyrrolidine-2-carboxylate (500 mg, 629.57 mol, 1 eq), TEA (318.53 mg, 3.15 mmol, 438.14 μL, 5 eq) and DMAP (38.46 mg, 314.79 mol, 0.5 eq) in DCM (10 mL) was added 3-(dimethylamino)propanoyl chloride (433.28 mg, 2.52 mmol, 4 eq, HCl) under N₂ at 0° C., and then the mixture was stirred at 20° C. for 1 hour. The mixture was added into saturated NaHCO₃ (20 mL), and extracted with EtOAc (10 mL×3). Theorganic layer was washed with brine (10 mL×2), dried over Na₂SO₄, filtered, and concentrated under reduced pressure. The residue was purified by column chromatography (SiO₂, petroleum ether/ethyl acetate=10/1 to 0/1) to give the product. The product was dissolved with petroleum ether (2 mL), and washed with ACN (2 mL×2). The petroleum ether phase was concentrated under reduced pressure to give [8-(1-octylnonoxy)-8-oxo-octyl](2S)-4-[3-(dimethylamino)propanoyloxy]-1-(6-oxo-6-undecoxy-hexanoyl)pyrrolidine-2-carboxylate (200 mg, 223.88 mol, 35.56% yield, 100% purity) as yellow oil.

¹H NMR (400 MHz, CDCl₃), 5.33-5.37 (m, 1H), 4.80-4.90 (m, 1H), 4.40-4.75 (m, 1H), 4.11-4.15 (m, 2H), 4.00-4.10 (m, 2H), 3.85-3.88 (m, 1H), 3.61-3.70 (m, 1H), 2.52-2.65 (m, 2H), 2.40-2.50 (m, 3H), 2.15-2.38 (m, 13H), 1.66-1.70 (m, 8H), 1.45-1.53 (m, 6H), 1.23-1.38 (m, 46H), 0.86-0.91 (m, 9H). LCMS: (M+H⁺): 893.7 @13.385/13.687 minutes.

### 8.11. Synthesis of Compound 2305

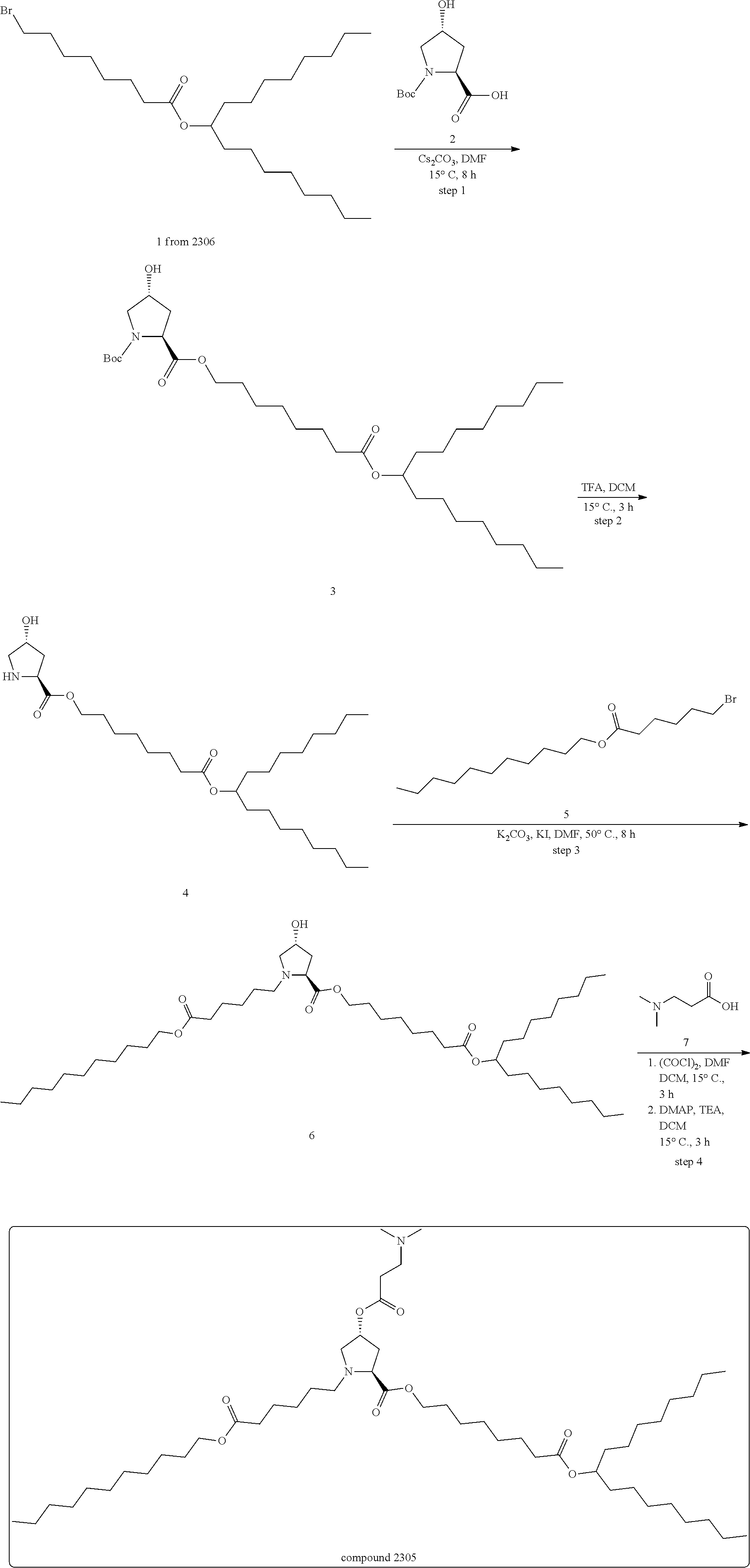

### Step 1

A mixture of (2S,4R)-1-tert-butoxycarbonyl-4-hydroxy-pyrrolidine-2-carboxylic acid (835.02 mg, 3.61 mmol, 1 eq), 1-octylnonyl 8-bromooctanoate (2 g, 4.33 mmol, 1.2 eq), Cs₂CO₃ (2.59 g, 7.94 mmol, 2.2 eq) in DMF (30 mL) was degassed and purged with N₂ for 3 times, and then the mixture was stirred at 15° C. for 8 hours under N₂ atmosphere. The reaction mixture was quenched by addition of 100 mL H₂O at 15° C., and then extracted with 300 mL EtOAc (100 mL×3). The combined organic layers were washed with 200 mL brine (100 mL×2), dried over Na₂SO₄, filtered, and concentrated under reduced pressure to give a residue. The residue was purified by column chromatography (SiO₂, petroleum ether/ethyl acetate=20/1 to 3/1) to give O1-tert-butyl O2-[8-(1-octylnonoxy)-8-oxo-octyl](2S,4R)-4-hydroxypyrrolidine-1,2-dicarboxylate (11 g, 17.71 mmol, 98.08% yield, 98.5% purity) as colorless oil.

¹H NMR (400 MHz, CDCl₃), 4.85-4.88 (m, 1H), 4.36-4.51 (m, 2H), 3.64-3.68 (m, 1H), 3.42-3.56 (m, 1H), 2.06-2.33 (m, 4H), 1.60-1.64 (m, 5H), 1.20-1.55 (m, 45H), 0.88 (t, J=6.4 Hz, 6H).

### Step 2

To a solution of O1-tert-butyl O2-[8-(1-octylnonoxy)-8-oxo-octyl](2S,4R)-4-hydroxypyrrolidine-1,2-dicarboxylate (8 g, 13.07 mmol, 1 eq) in DCM (60 mL) was added TFA (46.20 g, 405.19 mmol, 30 mL, 30.99 eq). The mixture was stirred at 15° C. for 3 hours. The reaction mixture was quenched by addition of 50 mL NaHCO₃ at 15° C., and then extracted with 150 mL EtOAc (50 mL×3). The combined organic layers were washed with 100 mL brine (50 mL×2), dried over Na₂SO₄, filtered, and concentrated under reduced pressure to give a residue to give [8-(1-octylnonoxy)-8-oxo-octyl](2S,4R)-4-hydroxypyrrolidine-2-carboxylate (5 g, 9.77 mmol, 74.73% yield) as colorless oil.

### Step 3

To a solution of undecyl 6-bromohexanoate (819.14 mg, 2.34 mmol, 1.2 eq) and [8-(1-octylnonoxy)-8-oxo-octyl](2S,4R)-4-hydroxypyrrolidine-2-carboxylate (1 g, 1.95 mmol, 1 eq) in DMF (15 mL) was added K₂CO₃ (810.18 mg, 5.86 mmol, 3 eq) and KI (162.18 mg, 976.99 mol, 0.5 eq). The mixture was stirred at 50° C. for 8 hours. The reaction mixture was quenched by addition of 50 mL H₂O at 15° C., and then extracted with 150 mL EtOAc (50 mL×3). The combined organic layers were washed with 100 mL brine (50 mL×2), dried over Na₂SO₄, filtered, and concentrated under reduced pressure to give a residue. The residue was purified by column chromatography (SiO₂, petroleum ether/ethyl acetate=1/0 to 8/1) and purified by prep-HPLC (column: Phenomenex Luna C18 100×30 mm×5 m; mobile phase: [water(HCl)-ACN]; B %: 50%-80%, 10 minutes) to give a solution. The solution of was added saturated NaHCO₃ (200 mL), and then extracted with EtOAc (50 mL×2). The organic layer was washed with brine (20 mL×2), dried over Na₂SO₄, filtered, and concentrated under reduced pressure to give [8-(1-octylnonoxy)-8-oxo-octyl](2S,4R)-4-hydroxy-1-(6-oxo-6-undecoxy-hexyl)pyrrolidine-2-carboxylate (3.6 g, 4.60 mmol, 78.52% yield, 99.76% purity) as yellow oil.

¹H NMR (400 MHz, CDCl₃), 4.85-4.90 (m, 1H), 4.51 (brs, 1H), 4.04-4.15 (m, 4H), 3.47-3.49 (m, 2H), 2.57-2.78 (m, 2H), 2.26-2.32 (m, 6H), 1.50-1.66 (m, 17H), 1.26-1.35 (m, 47H), 0.88 (t, J=6.8 Hz, 9H).

### Step 4

To a solution of 3-(dimethylamino)propanoic acid (300 mg, 1.95 mmol, 1 eq, HCl) in DCM (5 mL) was added (COCl)₂ (743.67 mg, 5.86 mmol, 512.88 μL, 3 eq) and DMF (14.28 mg, 195.30 mol, 15.03 μL, 0.1 eq). The mixture was stirred at 15° C. for 3 hours. The reaction mixture was concentrated under reduced pressure to give 3-(dimethylamino) propanoyl chloride (336 mg, 1.95 mmol, 99.99% yield, HCl) as a yellow solid. The 3-(dimethylamino)propanoyl chloride (330.78 mg, 1.92 mmol, 3 eq, HCl) was added to a solution of [8-(1-octylnonoxy)-8-oxo-octyl](2S,4R)-4-hydroxy-1-(6-oxo-6-undecoxy-hexyl)pyrrolidine-2-carboxylate (500 mg, 640.85 mol, 1 eq), TEA (324.24 mg, 3.20 mmol, 445.99 μL, 5 eq), and DMAP (39.15 mg, 320.43 mol, 0.5 eq) in DCM (10 mL). The mixture was stirred at 15° C. for 3 hours. The reaction mixture was quenched by addition of 10 mL aqueous NaHCO₃ at 15° C., and then extracted with 30 mL EtOAc (10 mL×3). The combined organic layers were washed with 20 mL brine (10 mL×2), dried over Na₂SO₄, filtered, and concentrated under reduced pressure to give a residue. The residue was purified by column chromatography (SiO₂, petroleum ether/ethyl acetate=1/0 to 3/1), and purified by prep-HPLC (column: Phenomenex Luna C18 100×30 mm×5 m; mobile phase: [water(HCl)-ACN]; B %: 45%-75%, 10 minutes) to give a solution. The solution of was added saturated NaHCO₃ (200 mL), and then extracted with EtOAc (50 mL×2). The organic layer was washed with brine (20 mL×2), dried over Na₂SO₄, filtered, and concentrated under reduced pressure to give [8-(1-octylnonoxy)-8-oxo-octyl](2S,4R)-4-[3-(dimethylamino)propanoyloxy]-1-(6-oxo-6-undecoxy-hexyl)pyrrolidine-2-carboxylate (79 mg, 89.39 μmol, 13.95% yield, 99.5% purity) as a colorless oil.

¹H NMR (400 MHz, CDCl₃), 5.24-5.29 (m, 1H), 4.84-4.88 (m, 1H), 4.04-4.13 (m, 4H), 3.44-3.54 (m, 2H), 2.26-3.03 (m, 16H), 2.08-2.22 (m, 1H), 1.60-1.65 (m, 10H), 1.50-1.52 (m, 6H), 1.26-1.37 (m, 48H), 0.88 (t, J=6.4 Hz, 9H). LCMS: (M+H⁺): 879.8 @10.515 minutes

### 8.12. Synthesis of Compound 2306

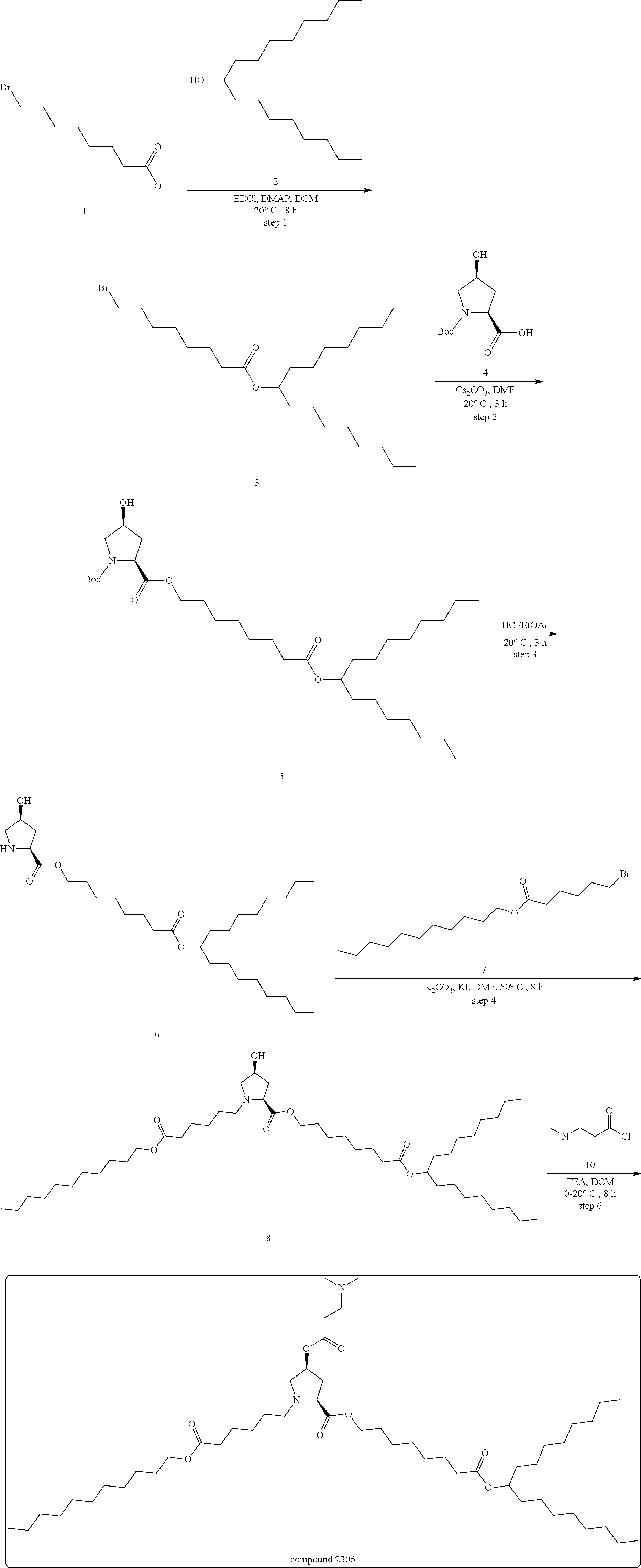

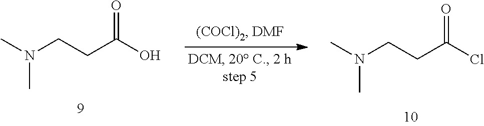

### Step 1

To a solution of heptadecan-9-ol (5 g, 19.50 mmol, 1 eq) and 8-bromooctanoic acid (4.57 g, 20.47 mmol, 1.05 eq) in DCM (100 mL) was added DMAP (1.19 g, 9.75 mmol, 0.5 eq) and EDCI (4.48 g, 23.39 mmol, 1.2 eq). The mixture was stirred at 20° C. for 8 hours. The mixture was added into H₂O (100 mL), and extracted with EtOAc (100 mL×3). The organic layer was washed with brine (100 mL×2), dried over Na₂SO₄, filtered, and concentrated under reduced pressure. The residue was purified by column chromatography (SiO₂, petroleum ether/ethyl acetate=1/0 to 5/1) to give 1-octylnonyl 8-bromooctanoate (30 g, 65.00 mmol, 83.35% yield,—purity) as colorless oil.

### Step 2

A mixture of (2S,4S)-1-tert-butoxycarbonyl-4-hydroxy-pyrrolidine-2-carboxylic acid (4.55 g, 19.70 mmol, 1 eq), 1-octylnonyl 8-bromooctanoate (10 g, 21.67 mmol, 1.1 eq), Cs₂CO₃ (14.12 g, 43.33 mmol, 2.2 eq) in DMF (100 mL) was stirred at 20° C. for 3 hours under N₂ atmosphere. The reaction mixture was quenched by addition of 100 mL H₂O at 0° C. The mixture was extracted with EtOAc 300 mL (100 mL×3) and the combined organic layers were dried over Na₂SO₄, filtered, and concentrated under reduced pressure to give a residue. The residue was purified by column chromatography (SiO₂, petroleum ether/ethyl acetate=10/1 to 3/1) to give O1-tert-butyl O2-[8-(1-octylnonoxy)-8-oxo-octyl](2S,4S)-4-hydroxypyrrolidine-1,2-dicarboxylate (10 g, 16.34 mmol, 82.97% yield, 100% purity) as a white solid.

¹H NMR (400 MHz, CDCl₃), 4.85-4.88 (m, 1H), 4.15-4.37 (m, 4H), 3.54-3.70 (m, 2H), 2.28-2.32 (m, 3H), 2.05-2.07 (m, 1H), 1.62-1.67 (m, 4H), 1.43-1.56 (m, 11H), 1.26-1.35 (m, 32H), 0.88 (t, J=6.4 Hz, 6H).

### Step 3

To a solution of O1-tert-butyl O2-[8-(1-octylnonoxy)-8-oxo-octyl](2S,4S)-4-hydroxypyrrolidine-1,2-dicarboxylate (2 g, 3.27 mmol, 1 eq) in EtOAc (50 mL) was added HCl/EtOAc (4 M, 6.21 mL, 7.60 eq). The mixture was stirred at 20° C. for 3 hours. The reaction mixture was adjusted to pH=7 with aqueous saturated NaHCO₃ and extracted with 600 mL EtOAc (200 mL×3), dried over Na₂SO₄, filtered, and concentrated under reduced pressure to give a residue. The residue was purified by column chromatography (SiO₂, petroleum ether/ethyl acetate=4/1 to ethyl acetate/NH₃·H2O=30/1) to give [8-(1-octylnonoxy)-8-oxo-octyl](2S,4S)-4-hydroxypyrrolidine-2-carboxylate (6 g, 11.72 mmol, 71.74% yield, 100% purity) as yellow oil.

### Step 4

To a solution of undecyl 6-bromohexanoate (409.57 mg, 1.17 mmol, 1.2 eq) and [8-(1-octylnonoxy)-8-oxo-octyl](2S,4S)-4-hydroxypyrrolidine-2-carboxylate (0.5 g, 976.99 mol, 1 eq) in DMF (20 mL) was added K₂CO₃ (405.09 mg, 2.93 mmol, 3 eq) and KI (81.09 mg, 488.50 mol, 0.5 eq). The mixture was stirred at 50° C. for 8 hours. The reaction mixture was quenched by addition of 50 mL H₂O at 15° C., and then extracted with 150 mL EtOAc (50 mL×3). The combined organic layers were washed with 100 mL brine (50 mL×2), dried over Na₂SO₄, filtered, and concentrated under reduced pressure to give a residue. The residue was purified by column chromatography (SiO₂, petroleum ether/ethyl acetate=1/0 to 5/1). And then the residue was purified by prep-HPLC (column: Phenomenex Luna C18 100×30 mm×5 μm; mobile phase: [water(HCl)-ACN]; B %: 50%-80%, 10 minutes). The mixture was added to 500 mL saturated NaHCO₃, and extracted with EtOAc (100 mL×3). The organic layer was washed with brine (50 mL×3), dried over Na₂SO₄, filtered, and concentrated under reduced pressure to give [8-(1-octylnonoxy)-8-oxo-octyl](2S,4S)-4-hydroxy-1-(6-oxo-6-undecoxy-hexyl)pyrrolidine-2-carboxylate (600 mg, 769.03 mol, 78.71% yield) as a yellow oil.

¹H NMR (400 MHz, CDCl₃), 4.77-4.80 (m, 1H), 4.18 (brs, 1H), 4.05 (t, J=6.8 Hz, 2H), 3.98 (t, J=6.4 Hz, 2H), 2.98-3.16 (m, 2H), 2.49-2.60 (m, 2H), 2.40-2.42 (m, 1H), 2.13-2.23 (m, 5H), 1.81-1.85 (m, 1H), 1.53-1.57 (m, 8H), 1.38-1.43 (m, 6H), 1.18-1.26 (m, 48H), 0.80 (t, J=6.0 Hz, 9H).

### Step 5

To a solution of 3-(dimethylamino)propanoic acid (500 mg, 3.26 mmol, 1 eq, HCl) in DCM (5 mL) was added (COCl)₂ (1.24 g, 9.77 mmol, 854.80 μL, 3 eq) and DMF (23.79 mg, 325.51 mol, 25.04 μL, 0.1 eq). The mixture was stirred at 20° C. for 2 hours. The reaction mixture was concentrated under reduced pressure to give 3-(dimethylamino) propanoyl chloride (560 mg, 3.25 mmol, 99.99% yield, HCl) as a yellow solid.

### Step 6

To a solution of 3-(dimethylamino)propanoyl chloride (352.83 mg, 2.05 mmol, 4 eq, HCl) in DCM (10 mL) was added TEA (259.39 mg, 2.56 mmol, 356.79 μL, 5 eq) and DMAP (31.32 mg, 256.34 mol, 0.5 eq) and [8-(1-octylnonoxy)-8-oxo-octyl](2S,4S)-4-hydroxy-1-(6-oxo-6-undecoxy-hexyl)pyrrolidine-2-carboxylate (400.00 mg, 512.68 mol, 1 eq) at 0° C. The mixture was stirred at 20° C. for 8 hours. The reaction mixture was quenched by addition of 30 mL H₂O at 15° C., and then extracted with 60 mL EtOAc (20 mL×3). The combined organic layers were washed with 40 mL brine (20 mL×2), dried over Na₂SO₄, filtered, and concentrated under reduced pressure to give a residue. The residue was purified by column chromatography (SiO₂, petroleum ether/ethyl acetate=1/0 to 0/1) to give a residue. The residue was purified by prep-HPLC (column: Phenomenex Luna C18 100×30 mm×5 m; mobile phase: [water(HCl)-ACN]; B %: 45%-75%, 10 minutes) and prep-TLC (SiO₂, EtOAc:MeOH=3:1) to give [8-(1-octylnonoxy)-8-oxo-octyl](2S,4S)-4-[3-(dimethylamino)propanoyloxy]-1-(6-oxo-6-undecoxy-hexyl)pyrrolidine-2-carboxylate (20.7 mg, 23.54 mol, 60.88% yield) as a yellow oil.

¹H NMR (400 MHz, CDCl₃), 5.11-5.15 (m, 1H), 4.77-4.80 (m, 1H), 3.96-4.06 (m, 4H), 3.01-3.18 (m, 2H), 2.49-2.67 (m, 6H), 2.19-2.22 (m, 10H), 1.96-1.98 (m, 1H), 1.52-1.77 (m, 10H), 1.42-1.44 (m, 6H), 1.18-1.26 (m, 48H), 0.80 (t, J=6.4 Hz, 9H).

LCMS: (M+H⁺): 879.8 @8.994 minutes.

### 8.13. Synthesis of Compound 2307

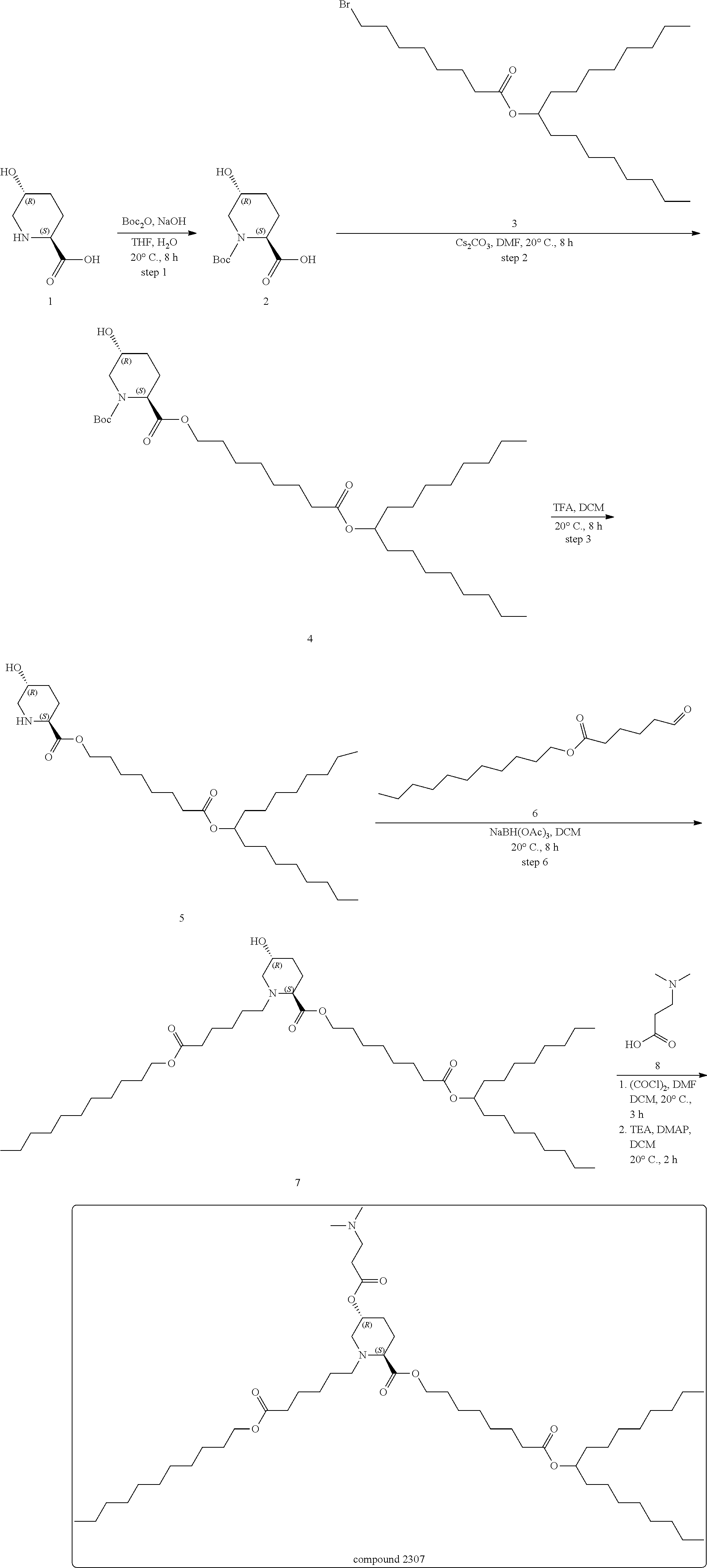

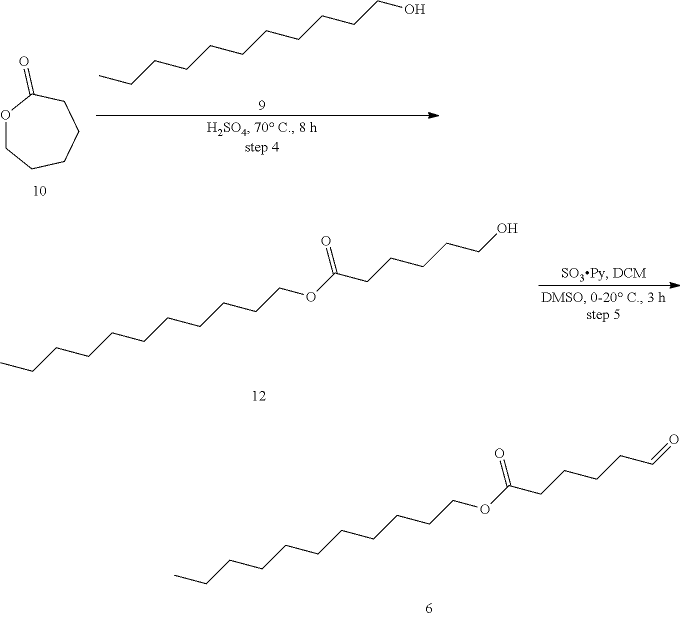

### Step 1

To a solution of (2S,5R)-5-hydroxypiperidine-2-carboxylic acid (0.5 g, 2.75 mmol, 1 eq, HCl) in THF (25 mL) was added aq.NaOH (3.30 g, 8.26 mmol, 10% purity, 3 eq) and Boc₂O (721.01 mg, 3.30 mmol, 758.96 μL, 1.2 eq). The mixture was stirred at 20° C. for 8 hours. The mixture was added into H₂O (20 mL), and extracted with EtOAc (10 mL×3). The organic layer was washed with brine (20 mL×2), dried over Na₂SO₄, filtered, and concentrated under reduced pressure to give (2S,5R)-1-tert-butoxycarbonyl-5-hydroxy-piperidine-2-carboxylic acid (500 mg, crude) as colorless oil.

### Step 2

To a solution of (2S,5R)-1-tert-butoxycarbonyl-5-hydroxy-piperidine-2-carboxylic acid (500 mg, 2.04 mmol, 1 eq) and 1-octylnonyl 8-bromooctanoate (1.13 g, 2.45 mmol, 1.2 eq) in DMF (10 mL) was added Cs₂CO₃ (1.46 g, 4.48 mmol, 2.2 eq). The mixture was stirred at 20° C. for 8 hours. The mixture was added into H₂O (20 mL), and extracted with EtOAc (20 mL×3). The organic layer was washed with brine (20 mL×2), dried over Na₂SO₄, filtered, and concentrated under reduced pressure. The residue was purified by column chromatography (SiO₂, petroleum ether/ethyl acetate=1/0 to 5/1) to give O1-tert-butyl O2-[8-(1-octylnonoxy)-8-oxo-octyl](2S,5R)—S-hydroxypiperidine-1,2-dicarboxylate (500 mg, 798.83 mol, 39.19% yield) as yellow oil.

¹H NMR (400 MHz, CDCl₃), 4.75-4.93 (m, 2H), 3.95-4.05 (m, 2H), 3.09-3.24 (m, 2H), 2.28 (t, J=7.2 Hz, 2H), 1.95-2.25 (m, 2H), 1.75-1.85 (m, 1H), 1.60-1.73 (m, 5H), 1.43-1.56 (m, 13H), 1.20-1.40 (m, 30H), 0.88 (t, J=6.4 Hz, 6H).

### Step 3

To a solution of O1-tert-butyl O2-[8-(1-octylnonoxy)-8-oxo-octyl](2S,5R)—S-hydroxypiperidine-1,2-dicarboxylate (500 mg, 798.83 mol, 1 eq) in DCM (10 mL) was added TFA (7.68 g, 67.31 mmol, 5 mL, 84.26 eq). The mixture was stirred at 20° C. for 2 hours. The mixture was concentrated under reduced pressure. The residue was dissolved with EtOAc (20 mL), and the organic layer was washed with saturated NaHCO₃ (20 mL×3) and brine (20 mL×2), dried over Na₂SO₄, filtered, and concentrated under reduced pressure. The residue was purified by column chromatography (SiO₂, petroleum ether/ethyl acetate=1/0 to 5/1) to give [8-(1-octylnonoxy)-8-oxo-octyl](2S,5R)—S-hydroxypiperidine-2-carboxylate (350 mg, crude) as yellow oil.

### Step 4

A solution of undecan-1-ol (6.04 g, 35.04 mmol, 2 eq), oxepan-2-one (2 g, 17.52 mmol, 1 eq) and H₂SO₄ (171.85 mg, 1.75 mmol, 93.40 μL, 0.1 eq) was stirred at 70° C. for 8 hours. The mixture was concentrated under reduced pressure. The residue was purified by column chromatography (SiO₂, petroleum ether/ethyl acetate=1/0 to 5/1) to give to compound undecyl 6-hydroxyhexanoate (2.6 g, 9.08 mmol, 51.80% yield) as yellow oil.

### Step 5

To a solution of undecyl 6-hydroxyhexanoate (2.5 g, 8.73 mmol, 1 eq) and TEA (8.83 g, 87.28 mmol, 12.15 mL, 10 eq) in DCM (100 mL) was added a solution of SO₃·Py (7.64 g, 48.00 mmol, 5.5 eq) in DMSO (25 mL) at 0° C. The mixture was stirred at 20° C. for 3 hours. The reaction mixture was diluted with 100 mL 0.1 M HCl and extracted with EtOAc (20 mL×3). Then organic layers was washed with brine (30 mL×3), dried over Na₂SO₄, filtered, and concentrated under reduced pressure to give a residue. The residue was purified by column chromatography (SiO₂, petroleum ether/ethyl acetate=1/0 to 5/1) to give undecyl 6-oxohexanoate (3.2 g, 11.25 mmol, 64.45% yield) as colourless oil.

### Step 6

To a solution of [8-(1-octylnonoxy)-8-oxo-octyl](2S,5R)—S-hydroxypiperidine-2-carboxylate (350 mg, 665.65 mol, 1 eq) and undecyl 6-oxohexanoate (227.20 mg, 798.78 mol, 1.2 eq) in DCM (10 mL) was added NaBH(OAc)₃ (423.24 mg, 2.00 mmol, 3 eq). The mixture was stirred at 20° C. for 8 hours. The mixture was added into H₂O (20 mL), and extracted with EtOAc (20 mL×3). The organic layer was washed with brine (20 mL×2), dried over Na₂SO₄, filtered, and concentrated under reduced pressure. The residue was purified by column chromatography (SiO₂, petroleum ether/ethyl acetate=1/0 to 5/1) to give [8-(1-octylnonoxy)-8-oxo-octyl](2S,5R)—S-hydroxy-1-(6-oxo-6-undecoxy-hexyl)piperidine-2-carboxylate (380 mg, 478.45 μmol, 71.88% yield) as yellow oil.

¹H NMR (400 MHz, CDCl₃), 4.85-4.90 (m, 1H), 4.04-4.12 (m, 4H), 3.86 (s, 1H), 3.21-3.25 (m, 2H), 2.55-2.65 (m, 1H), 2.40-2.50 (m, 1H), 2.20-2.40 (m, 6H), 1.95-2.10 (m, 1H), 1.70-1.85 (m, 2H), 1.55-1.70 (m, 8H), 1.43-1.56 (m, 7H), 1.20-1.40 (m, 48H), 0.89 (t, J=6.8 Hz, 9H).

### Step 7

To a solution of 3-(dimethylamino)propanoic acid (300 mg, 1.95 mmol, 1 eq, HCl) in DCM (5 mL) was added (COCl)₂ (991.60 mg, 7.81 mmol, 683.86 μL, 4 eq) and DMF (14.27 mg, 195.30 mol, 15.03 μL, 0.1 eq). The mixture was stirred at 20° C. for 3 hours. The mixture was concentrated under reduced pressure to give 3-(dimethylamino)propanoyl chloride (1.68 g, crude, HCl) as a yellow solid. The crude 3-(dimethylamino)propanoyl chloride (329.27 mg, 1.91 mmol, 4 eq, HCl) was added to a solution of [8-(1-octylnonoxy)-8-oxo-octyl](2S,5R)—S-hydroxy-1-(6-oxo-6-undecoxy-hexyl)piperidine-2-carboxylate (380 mg, 478.45 μmol, 1 eq), TEA (290.48 mg, 2.87 mmol, 399.56 μL, 6 eq) and DMAP (11.69 mg, 95.69 μmol, 0.2 eq) in DCM (10 mL) and stirred at 20° C. for 2 hours. The mixture was added into saturated NaHCO₃ (20 mL), and extracted with DCM (20 mL×2). The organic layer was washed with brine (20 mL×2), dried over Na₂SO₄, filtered, and concentrated under reduced pressure. The residue was purified by column chromatography (SiO₂, petroleum ether/ethyl acetate=1/0 to 0/1, added 5% NH₃·THF) to give [8-(1-octylnonoxy)-8-oxo-octyl](2S,5R)—S-[3-(dimethylamino)propanoyloxy]-1-(6-oxo-6-undecoxy-hexyl)piperidine-2-carboxylate (55 mg, 60.33 μmol, 12.61% yield, 98% purity) as colorless oil.

1H NMR (400 MHz, CDCl₃), 4.85-4.96 (m, 2H), 4.03-4.13 (m, 4H), 3.13-3.24 (m, 2H), 2.46-2.64 (m, 5H), 2.24-2.37 (m, 12H), 1.90-2.10 (m, 2H), 1.70-1.80 (m, 1H), 1.55-1.70 (m, 9H), 1.45-1.55 (m, 6H), 1.20-1.40 (m, 48H), 0.86-0.90 (m, 9H). LCMS: (M+H⁺): 893.8 @10.829 minutes.

### 8.14. Synthesis of Compound 2308

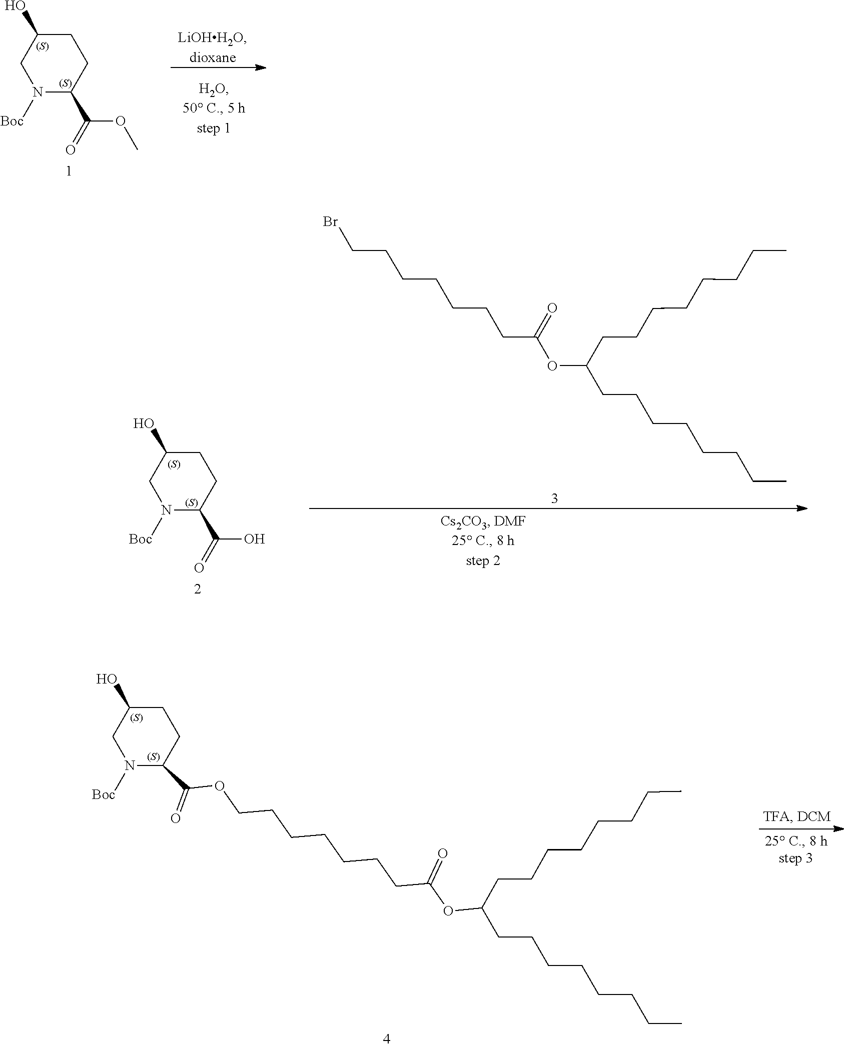

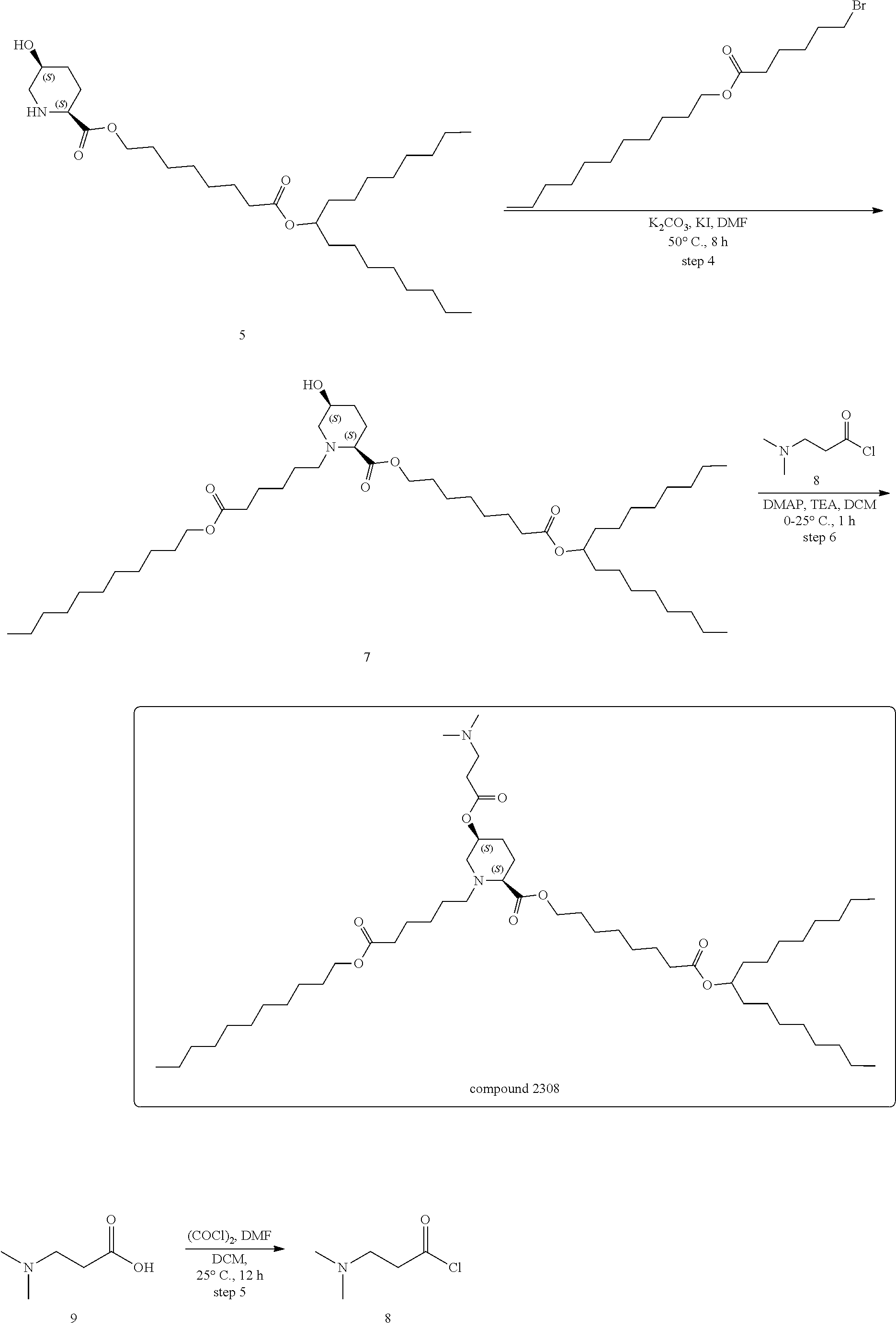

### Step 1

To a solution of O1-tert-butyl O2-methyl (2S,5S)-5-hydroxypiperidine-1,2-dicarboxylate (500 mg, 1.93 mmol, 1 eq) in dioxane (9 mL) and H₂O (3 mL) was added LiOH·H₂O (242.75 mg, 5.78 mmol, 3 eq). The mixture was stirred at 50° C. for 5 hours. The mixture was adjust to pH=5 with 1N HCl, and then extracted with 30 mL EtOAc (10 mL×3). The combined organic layers were washed with 30 mL saturated brine (10 mL×3), dried over Na₂SO₄, filtered, and concentrated under reduced pressure to give (2S,5S)-1-tert-butoxycarbonyl-5-hydroxy-piperidine-2-carboxylic acid (400 mg, crude) as a white solid. The crude product used into the next step without further purification.

### Step 2

To a solution of (2S,5S)-1-tert-butoxycarbonyl-5-hydroxy-piperidine-2-carboxylic acid (400 mg, 1.63 mmol, 1 eq) and 1-octylnonyl 8-bromooctanoate (828.00 mg, 1.79 mmol, 1.1 eq) in DMF (5 mL) was added Cs₂CO₃ (797.04 mg, 2.45 mmol, 1.5 eq). The mixture was stirred at 25° C. for 8 hours. The mixture was added into H₂O (20 mL), and extracted with EtOAc (20 mL×3). The organic layer was washed with brine (20 mL×2), dried over Na₂SO₄, filtered, and concentrated under reduced pressure. The residue was purified by column chromatography (SiO₂, petroleum ether/ethyl acetate=1/0 to 5/1) to give O1-tert-butyl O2-[8-(1-octylnonoxy)-8-oxo-octyl](2S,5S)—S-hydroxypiperidine-1,2-dicarboxylate (500 mg, 798.83 μmol, 48.98% yield) as a white solid.

¹H NMR (400 MHz, CDCl₃), 4.65-4.90 (m, 2H), 4.10-4.20 (m, 3H), 3.64 (brs, 1H), 2.60-2.85 (m, 1H), 2.28 (t, J=7.6 Hz, 3H), 1.90-2.05 (m, 1H), 1.70-1.85 (m, 2H), 1.58-1.65 (m, 4H), 1.40-1.55 (m, 13H), 1.20-1.40 (m, 32H), 0.89 (t, J=6.8 Hz, 6H).

### Step 3

To a solution of O1-tert-butyl O2-[8-(1-octylnonoxy)-8-oxo-octyl](2S,5S)—S-hydroxypiperidine-1,2-dicarboxylate (500 mg, 798.83 mol, 1 eq) in DCM (4 mL) was added TFA (3.08 g, 27.01 mmol, 2 mL, 33.81 eq). The mixture was stirred at 25° C. for 8 hours. The mixture was added into H₂O (20 mL), and extracted with EtOAc (20 mL×3). The organic layer was washed with brine (20 mL×2), dried over Na₂SO₄, filtered, and concentrated under reduced pressure. The residue was purified by column chromatography (SiO₂, petroleum ether/ethyl acetate=1/0 to 5/1) to give [8-(1-octylnonoxy)-8-oxo-octyl](2S,5S)—S-hydroxypiperidine-2-carboxylate (400 mg, 760.74 mol, 95.23% yield) as a white solid.

### Step 4

To a solution of [8-(1-octylnonoxy)-8-oxo-octyl](2S,5S)—S-hydroxypiperidine-2-carboxylate (350 mg, 665.65 mol, 1 eq), K₂CO₃ (276.00 mg, 2.00 mmol, 3 eq) and KI (22.10 mg, 133.13 μmol, 0.2 eq) in DMF (20 mL) was added undecyl 6-bromohexanoate (395.32 mg, 1.13 mmol, 1.7 eq). The mixture was stirred at 50° C. for 8 hours. The mixture was added into H₂O (20 mL), extracted with EtOAc (20 mL×3), and the organic layer was washed with brine (20 mL×2), dried over Na₂SO₄, filtered, and concentrated under reduced pressure. The residue was purified by column chromatography (SiO₂, petroleum ether/ethyl acetate=1/0 to 5/1) to give [8-(1-octylnonoxy)-8-oxo-octyl](2S,5S)—S-hydroxy-1-(6-oxo-6-undecoxy-hexyl)piperidine-2-carboxylate (480 mg, 604.36 mol, 90.79% yield) as a white solid.

¹H NMR (400 MHz, CDCl₃), 4.80-4.90 (m, 1H), 4.04-4.15 (m, 4H), 3.75-3.88 (m, 1H), 2.90-3.15 (m, 2H), 2.20-2.60 (m, 8H), 1.95-2.05 (m, 1H), 1.60-1.75 (m, 11H), 1.45-1.53 (m, 6H), 1.20-1.40 (m, 48H), 0.86-0.91 (m, 9H).

### Step 5

To a solution of 3-(dimethylamino)propanoic acid (170 mg, 1.11 mmol, 1 eq, HCl) in DCM (5 mL) was added (COCl)₂ (561.90 mg, 4.43 mmol, 387.52 μL, 4 eq) and DMF (8.09 mg, 110.67 mol, 8.51 μL, 0.1 eq). The mixture was stirred at 25° C. for 12 hours. The mixture was concentrated under reduced pressure to give 3-(dimethylamino)propanoyl chloride (380 mg, crude, HCl) as a yellow solid.

### Step 6

To a solution of [8-(1-octylnonoxy)-8-oxo-octyl](2S,5S)—S-hydroxy-1-(6-oxo-6-undecoxy-hexyl)piperidine-2-carboxylate (200 mg, 251.81 mol, 1 eq), DMAP (15.38 mg, 125.91 μmol, 0.5 eq) and TEA (127.41 mg, 1.26 mmol, 175.25 μL, 5 eq) in DCM (10 mL) was added 3-(dimethylamino)propanoyl chloride (173.30 mg, 1.01 mmol, 4 eq, HCl) under N₂ at 0° C., and then the mixture was stirred at 25° C. for 1 hour. The mixture was added into saturated NaHCO₃ (20 mL), and extracted with EtOAc (10 mL×3). The organic layer was washed with brine (10 mL×2), dried over Na₂SO₄, filtered, and concentrated under reduced pressure. The residue was purified by column chromatography (SiO₂, petroleum ether/ethyl acetate=10/1 to 0/1) and further purify by prep-TLC (SiO₂, ethyl acetate/MeOH=0:1, added 3% NH₃·H₂O) to give [8-(1-octylnonoxy)-8-oxo-octyl](2S,5S)—S-[3-(dimethylamino)propanoyloxy]-1-(6-oxo-6-undecoxy-hexyl)piperidine-2-carboxylate (158 mg, 175.09 μmol, 34.77% yield, 99% purity) as yellow oil.

¹H NMR (400 MHz, CDCl₃), 4.83-4.89 (m, 2H), 4.00-4.12 (m, 4H), 3.55 (t, J=4.4 Hz, 1H), 2.93-2.98 (m, 1H), 2.45-2.80 (m, 7H), 2.20-2.35 (m, 10H), 1.95-2.10 (m, 1H), 1.75-1.90 (m, 2H), 1.60-1.65 (m, 7H), 1.40-1.55 (m, 8H), 1.20-1.40 (m, 48H), 0.86-0.91 (m, 9H).

LCMS (M+H⁺): 893.7.

### 8.15. Synthesis of Compound 2309

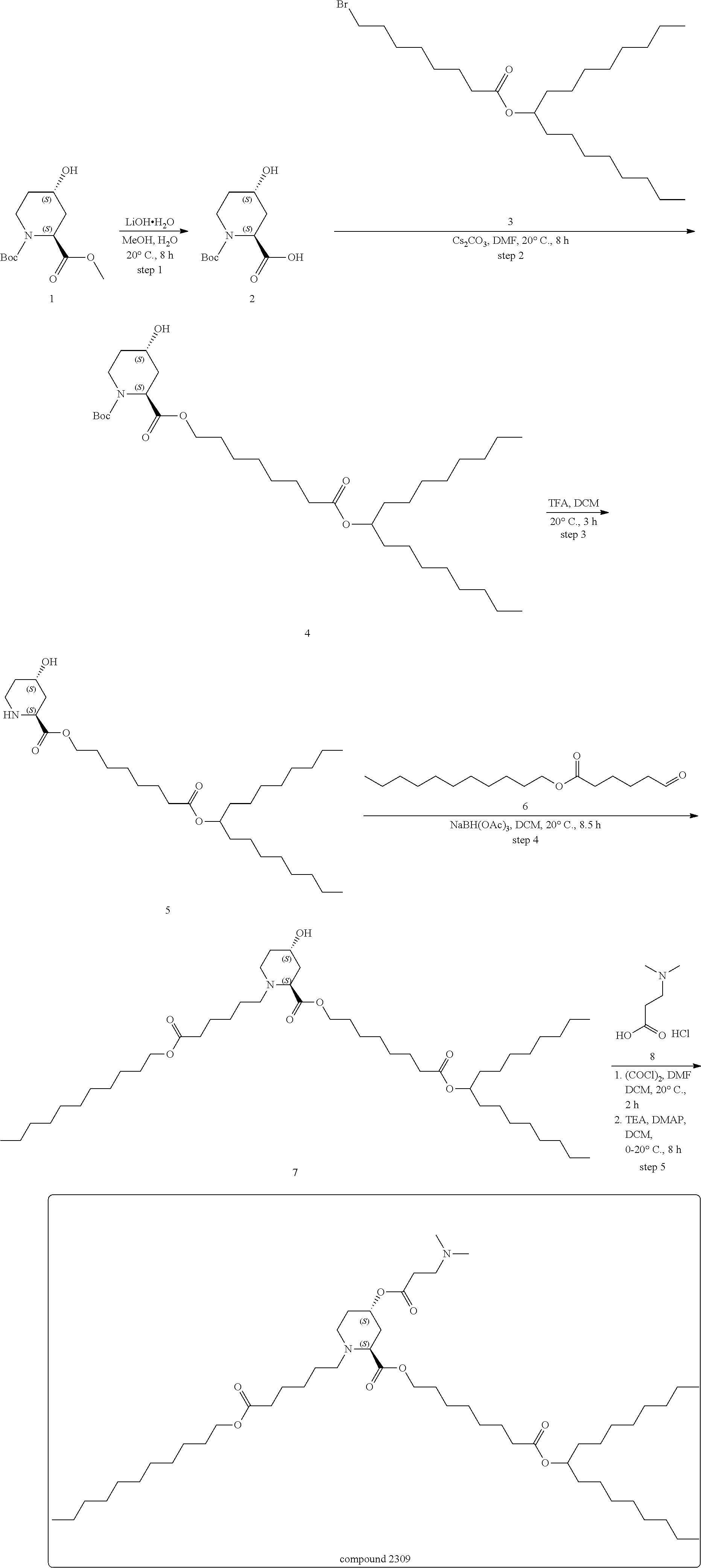

### Step 1

To a solution of O1-tert-butyl O2-methyl (2S,4S)-4-hydroxypiperidine-1,2-dicarboxylate (2 g, 7.71 mmol, 1 eq) in MeOH (12 mL) and H₂O (6 mL) was added LiOH·H₂O (647.34 mg, 15.43 mmol, 2 eq). The mixture was stirred at 20° C. for 8 hours. The mixture was adjust to pH=3 with 1N HCl, and then extracted with 60 mL EtOAc (20 mL×3). The combined organic layers were washed with 40 mL saturated brine (20 mL×2), dried over Na₂SO₄, filtered, and concentrated under reduced pressure to give (2S,4S)-1-tert-butoxycarbonyl-4-hydroxy-piperidine-2-carboxylic acid (1.8 g, 7.34 mmol, 95.15% yield) as colorless oil.

¹H NMR (400 MHz, CDCl₃), 5.98-6.80 (m, 1H), 4.88-5.05 (m, 1H), 4.01-4.10 (m, 1H), 3.72-3.77 (m, 1H), 2.97-3.09 (m, 1H), 2.35-2.60 (m, 1H), 1.90-2.00 (m, 1H), 1.60-1.75 (m, 1H), 1.47 (s, 9H).

### Step 2

To a solution of (2S,4S)-1-tert-butoxycarbonyl-4-hydroxy-piperidine-2-carboxylic acid (1.8 g, 7.34 mmol, 1 eq) and 1-octylnonyl 8-bromooctanoate (4.06 g, 8.81 mmol, 1.2 eq) in DMF (30 mL) was added Cs₂CO₃ (5.26 g, 16.15 mmol, 2.2 eq). The mixture was stirred at 20° C. for 8 hours. The reaction mixture was quenched by addition of 30 mL H₂O at 0° C., and then extracted with 60 mL EtOAc (20 mL×3). The combined organic layers were washed with 60 mL saturated brine (30 mL×2), dried over Na₂SO₄, filtered, and concentrated under reduced pressure to give a residue. The residue was purified by column chromatography (SiO₂, petroleum ether/ethyl acetate=10/1 to 3/1) to give O1-tert-butyl O2-[8-(1-octylnonoxy)-8-oxo-octyl](2S,4S)-4-hydroxypiperidine-1,2-dicarboxylate (2 g, 3.20 mmol, 43.54% yield) as colorless oil.

### Step 3

To a solution of O1-tert-butyl O2-[8-(1-octylnonoxy)-8-oxo-octyl](2S,4S)-4-hydroxypiperidine-1,2-dicarboxylate (2 g, 3.20 mmol, 1 eq) in DCM (12 mL) was added TFA (6 mL). The mixture was stirred at 20° C. for 3 hours. The mixture was concentrated under reduced pressure to give a residue, then adjusted to pH=8 with saturated NaHCO₃, and extracted with 60 mL EtOAc (20 mL×3). The combined organic layers were dried over Na₂SO₄, filtered, and concentrated under reduced pressure to give a residue. The residue was purified by column chromatography (SiO₂, petroleum ether/ethyl acetate=10/1 to 0/1, added 0.5% NH₃·H₂O) to give [8-(1-octylnonoxy)-8-oxo-octyl](2S,4S)-4-hydroxypiperidine-2-carboxylate (1.4 g, crude) as colorless oil.

### Step 4

To a solution of [8-(1-octylnonoxy)-8-oxo-octyl](2S,4S)-4-hydroxypiperidine-2-carboxylate (1 g, 1.90 mmol, 1 eq) and undecyl 6-oxohexanoate (649.14 mg, 2.28 mmol, 1.2 eq) in DCM (10 mL). The mixture was stirred at 20° C. for 30 minutes. Then NaBH(OAc)₃ (1.21 g, 5.71 mmol, 3 eq) was added to the mixture. The mixture was stirred at 20° C. for 8 hours. The mixture was concentrated under reduced pressure to give a residue. The residue was purified by column chromatography (SiO₂, petroleum ether/ethyl acetate=50/1 to 1/1, added 0.5% NH₃·H₂O) to give [8-(1-octylnonoxy)-8-oxo-octyl](2S,4S)-4-hydroxy-1-(6-oxo-6-undecoxy-hexyl)piperidine-2-carboxylate (1.3 g, 1.64 mmol, 86.06% yield) as colorless oil.

¹H NMR (400 MHz, CDCl₃), 4.85-4.88 (m, 1H), 4.03-4.11 (m, 5H), 3.51 (t, J=4.8 Hz, 1H), 2.92-2.98 (m, 1H), 2.53-2.62 (m, 2H), 2.38-2.45 (m, 1H), 2.26-2.31 (m, 4H), 2.05-2.07 (m, 1H), 1.89-1.94 (m, 1H), 1.74-1.82 (m, 1H), 1.58-1.65 (m, 9H), 1.46-1.55 (m, 6H), 1.26-1.35 (m, 48H), 0.88 (t, J=6.8 Hz, 9H).

### Step 5

To a solution of 3-(dimethylamino)propanoic acid (0.7 g, 4.56 mmol, 1 eq, HCl) in DCM (10 mL) was added DMF (16.65 mg, 227.85 mol, 17.53 μL, 0.05 eq) and (COCl)₂ (694.10 mg, 5.47 mmol, 478.69 μL, 1.2 eq). The mixture was stirred at 20° C. for 2 hours. The mixture was concentrated under reduced pressure to give 3-(dimethylamino)propanoyl chloride (780 mg, crude, HCl) as crude product. The crude 3-(dimethylamino)propanoyl chloride (758.19 mg, 4.41 mmol, 5 eq, HCl) was added to a solution of [8-(1-octylnonoxy)-8-oxo-octyl](2S,4S)-4-hydroxy-1-(6-oxo-6-undecoxy-hexyl)piperidine-2-carboxylate (700 mg, 881.35 μmol, 1 eq), TEA (891.83 mg, 8.81 mmol, 1.23 mL, 10 eq) and DMAP (53.84 mg, 440.68 μmol, 0.5 eq) in DCM (10 mL). The mixture was stirred at 20° C. for 8 hours. The reaction mixture was quenched by addition of 10 mL H₂O at 0° C., and then extracted with 30 mL EtOAc (10 mL×3). The combined organic layers were washed with saturated brine 20 mL (10 mL×2), dried over Na₂SO₄, filtered, and concentrated under reduced pressure to give a residue. The residue was purified by column chromatography (SiO₂, petroleum ether/ethyl acetate=20/1 to 0/1, added 0.5% NH₃·H₂O) to give [8-(1-octylnonoxy)-8-oxo-octyl](2S,4S)-4-[3-(dimethylamino)propanoyloxy]-1-(6-oxo-6-undecoxy-hexyl)piperidine-2-carboxylate (175 mg, 186.09 mol, 21.11% yield, 96% purity) as colorless oil.

¹H NMR (400 MHz, CDCl₃), 5.02-5.06 (m, 1H), 4.85-4.88 (m, 1H), 4.05-4.13 (m, 4H), 3.36-3.39 (m, 1H), 2.94-2.99 (m, 1H), 2.23-2.63 (m, 17H), 1.89-2.03 (m, 3H), 1.60-1.65 (m, 9H), 1.49-1.53 (m, 6H), 1.26-1.33 (m, 48H), 0.88 (t, J=6.8 Hz, 9H). LCMS: (M+H⁺): 893.8 @9.863 minutes

### 8.16. Synthesis of Compound 2310

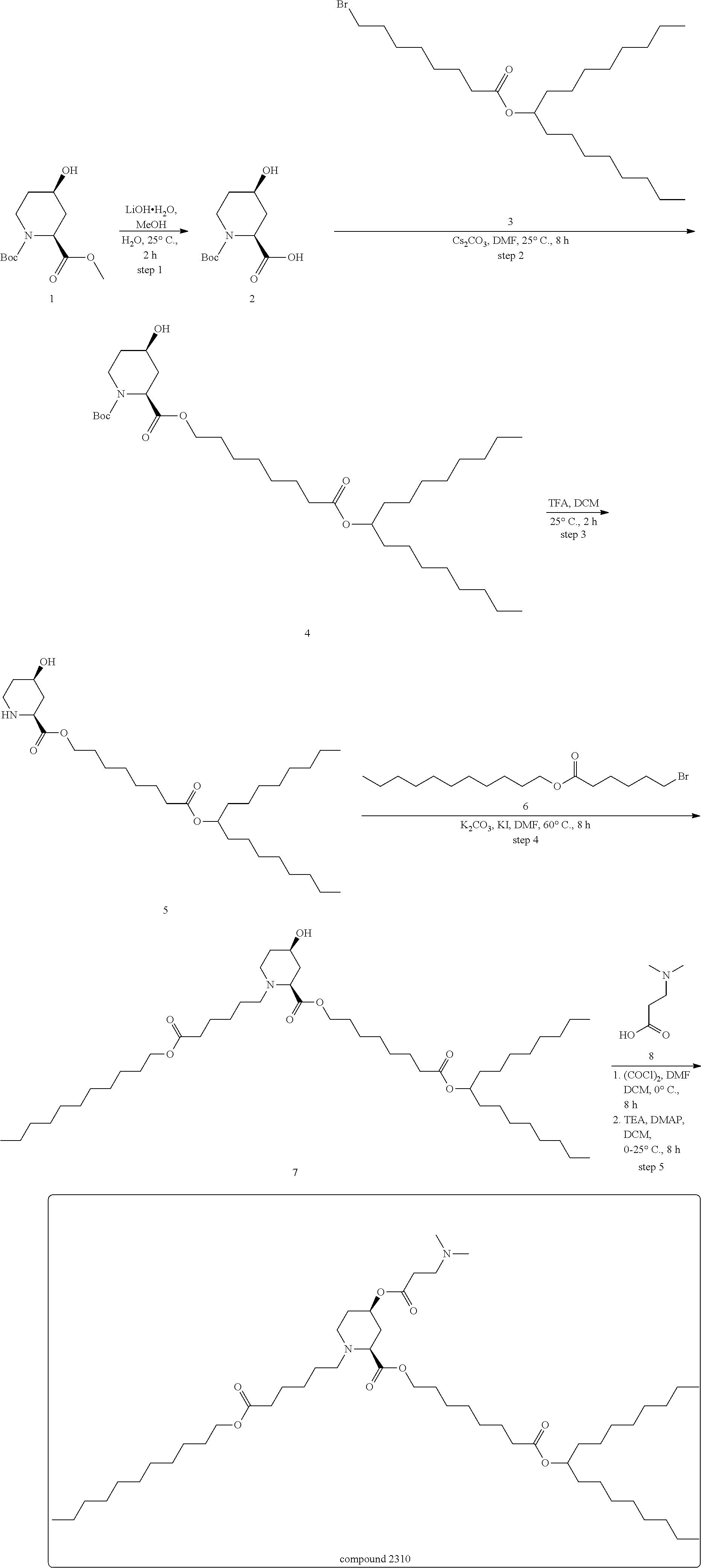

### Step 1

To a solution of O1-tert-butyl O2-methyl (2S,4R)-4-hydroxypiperidine-1,2-dicarboxylate (2 g, 7.71 mmol, 1 eq) in MeOH (12 mL) and H₂O (6 mL) was added LiOH·H₂O (647.34 mg, 15.43 mmol, 2 eq). The mixture was stirred at 25° C. for 2 hours. The mixture was concentrated under reduced pressure to give a residue. Then the solution was adjusted to pH=3 with 1N HCl, and extracted with EtOAc 45 mL (15 mL×3). The combined organic layers were dried over Na₂SO₄, filtered, and concentrated under reduced pressure to give (2S,4R)-1-tert-butoxycarbonyl-4-hydroxy-piperidine-2-carboxylic acid (1.6 g, 6.52 mmol, 84.58% yield) as colorless oil and used into the next step without further purification.

### Step 2

To a solution of (2S,4R)-1-tert-butoxycarbonyl-4-hydroxy-piperidine-2-carboxylic acid (1.6 g, 6.52 mmol, 1 eq) and 1-octylnonyl 8-bromooctanoate (3.61 g, 7.83 mmol, 1.2 eq) in DMF (15 mL) was added Cs₂CO₃ (4.68 g, 14.35 mmol, 2.2 eq). The mixture was stirred at 25° C. for 8 hours. The reaction mixture was quenched by addition of 20 mL H₂O at 0° C., and then extracted with 60 mL EtOAc (20 mL×3). The combined organic layers were dried over Na₂SO₄, filtered, and concentrated under reduced pressure to give a residue. The residue was purified by column chromatography (SiO₂, petroleum ether/ethyl acetate=20/1 to 0/1) to give O1-tert-butyl O2-[8-(1-octylnonoxy)-8-oxo-octyl](2S,4R)-4-hydroxypiperidine-1,2-dicarboxylate (2 g, 3.20 mmol, 49.02% yield) as yellow oil.

¹H NMR (400 MHz, CDCl₃), 4.64-4.89 (m, 1H), 4.11-4.20 (m, 3H), 3.70-3.92 (m, 1H), 3.27-3.42 (m, 1H), 2.41-2.44 (m, 1H), 2.27-2.29 (m, 2H), 1.87-1.93 (m, 1H), 1.58-1.64 (m, 8H), 1.45-1.51 (m, 14H), 1.26-1.34 (m, 30H), 0.88 (t, J=6.8H, 6H).

### Step 3

To a solution of O1-tert-butyl O2-[8-(1-octylnonoxy)-8-oxo-octyl](2S,4R)-4-hydroxypiperidine-1,2-dicarboxylate (2 g, 3.20 mmol, 1 eq) in DCM (14 mL) was added TFA (7 mL). The mixture was stirred at 25° C. for 2 hours. The mixture was concentrated under reduced pressure, then was adjusted to pH=8 with saturated NaHCO₃, and extracted with EtOAc 90 mL (30 mL×3). The combined organic layers were dried over Na₂SO₄, filtered, and concentrated under reduced pressure to give a residue. The residue was purified by column chromatography (SiO₂, petroleum ether/ethyl acetate=10/1 to 0/1, added 3% NH₃·H₂O) to give [8-(1-octylnonoxy)-8-oxo-octyl](2S,4R)-4-hydroxypiperidine-2-carboxylate (1.5 g, 2.85 mmol, 89.29% yield) as yellow oil.

### Step 4

To a solution of [8-(1-octylnonoxy)-8-oxo-octyl](2S,4R)-4-hydroxypiperidine-2-carboxylate (500 mg, 950.93 μmol, 1 eq) in DMF (10 mL) was added undecyl 6-bromohexanoate (996.61 mg, 2.85 mmol, 3 eq), K₂CO₃ (394.27 mg, 2.85 mmol, 3 eq) and KI (157.86 mg, 950.93 μmol, 1 eq). The mixture was stirred at 60° C. for 8 hours. The reaction mixture was quenched by addition H₂O 60 mL, and then extracted with EtOAc (50 mL×3). The combined organic layers were dried over Na₂SO₄, filtered, and concentrated under reduced pressure to give a residue. The residue was purified by column chromatography (SiO₂, petroleum ether/ethyl acetate=10/1 to 0/1) to give [8-(1-octylnonoxy)-8-oxo-octyl](2S,4R)-4-hydroxy-1-(6-oxo-6-undecoxy-hexyl) piperidine-2-carboxylate (550 mg, 692.49 mol, 72.82% yield) as yellow oil.

¹H NMR (400 MHz, CDCl₃), 4.82-4.88 (m, 1H), 4.02-4.14 (m, 2H), 3.72-3.74 (m, 1H), 3.07-3.72 (m, 2H), 2.48-2.55 (m, 1H), 2.26-2.30 (m, 8H), 1.89-1.92 (m, 1H), 1.73-1.78 (m, 2H), 1.57-1.63 (m, 8H), 1.49-1.52 (m, 6H), 1.24-1.33 (m, 50H), 0.87 (t, J=6.4 Hz, 9H).

### Step 5

To a solution of 3-(dimethylamino)propanoic acid (440 mg, 2.86 mmol, 1 eq, HCl) in DCM (5 mL) was added DMF (10.47 mg, 143.22 mol, 11.02 μL, 0.05 eq) and oxalyl dichloride (436.29 mg, 3.44 mmol, 300.89 μL, 1.2 eq). The mixture was stirred at 0° C. for 8 hours. The reaction mixture was concentrated under reduced pressure to give 3-(dimethylamino)propanoyl chloride (492 mg, crude, HCl) as yellow oil. The crude oil residue was dissolved with DCM (5 mL), and then added into a solution of [8-(1-octylnonoxy)-8-oxo-octyl](2S,4R)-4-hydroxy-1-(6-oxo-6-undecoxy-hexyl)piperidine-2-carboxylate (500 mg, 629.54 mol, 1 eq), TEA (637.02 mg, 6.30 mmol, 876.23 μL, 10 eq) and DMAP (38.45 mg, 314.77 mol, 0.5 eq) in DCM (10 mL) at 0° C. The combined mixture was stirred at 25° C. for 8 hours. The reaction mixture was quenched by addition of 10 mL H₂O, and then extracted with 15 mL EtOAc (5 mL×3). The combined organic layers were dried over Na₂SO₄, filtered, and concentrated under reduced pressure to give a residue. The residue was purified by p-HPLC (column: Xselect CSH C18 100×30 mm×5 m; mobile phase: [H₂O(0.04% HCl)-THF:ACN=1:3]; gradient:30%-70% B over 10.0 minutes) to give [8-(1-octylnonoxy)-8-oxo-octyl](2S,4R)-4-[3-(dimethylamino)propanoyloxy]-1-(6-oxo-6-undecoxy-hexyl)piperidine-2-carboxylate (29 mg, 32.46 mol, 36.25% yield, HCl salt) as yellow oil.

1H NMR (400 MHz, CDCl₃), 12.86-13.00 (m, 2H), 5.16 (brs, 1H), 4.82-4.85 (m, 1H), 3.85-4.20 (m, 6H), 3.30 (brs, 4H), 2.71-3.12 (m, 10H), 2.50-2.54 (m, 1H), 2.26-2.32 (m, 4H), 2.13 (brs, 1H), 1.96 (brs, 1H), 1.2 (brs, 1H), 1.63-1.82 (m, 8H), 1.49-1.50 (m, 4H), 1.25-1.50 (m, 49H), 0.87 (t, J=6.4H, 9H). LCMS (CAD): (M+H⁺): 893.3 @9.371 minutes.

### 8.17. Synthesis of Compound 2329

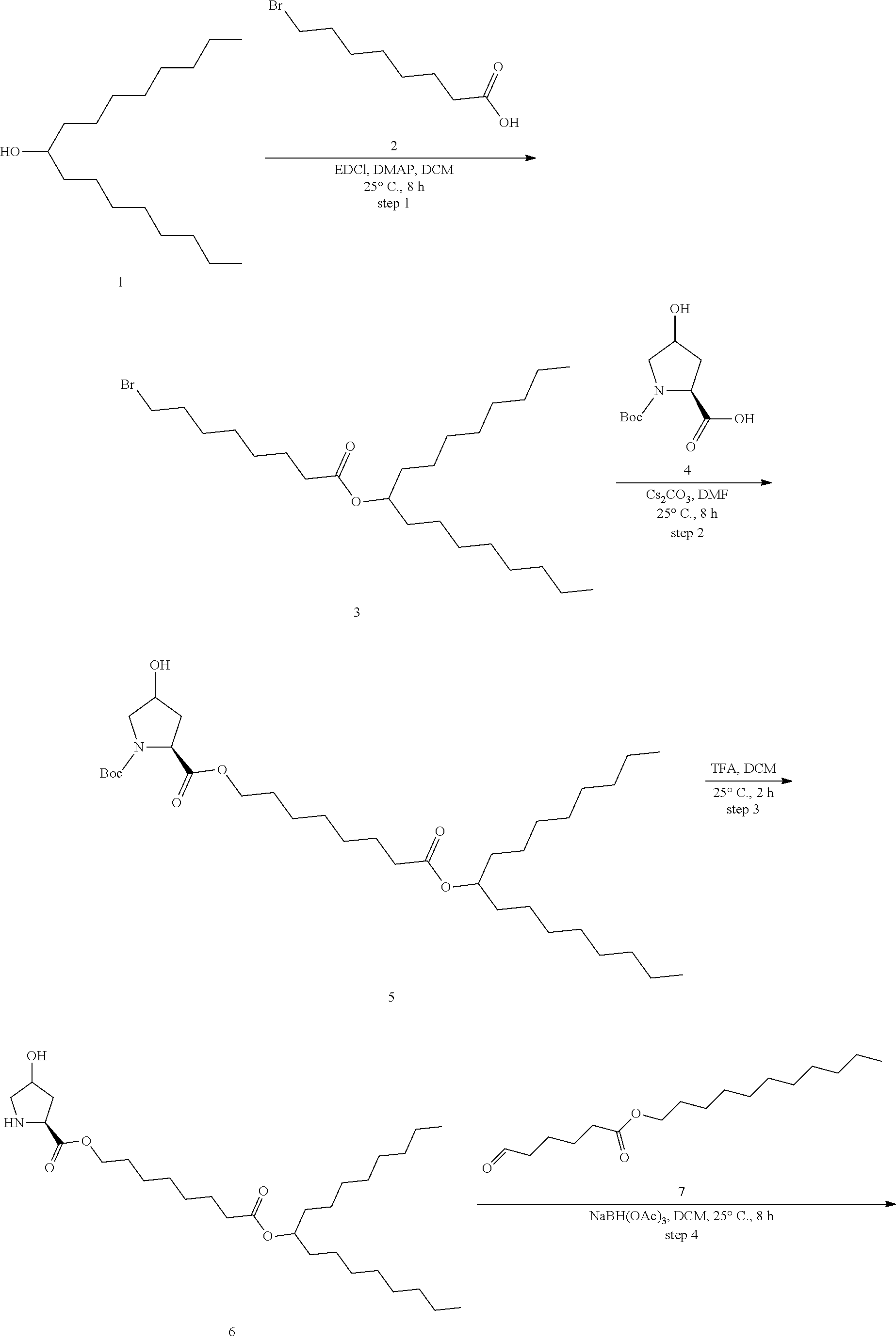

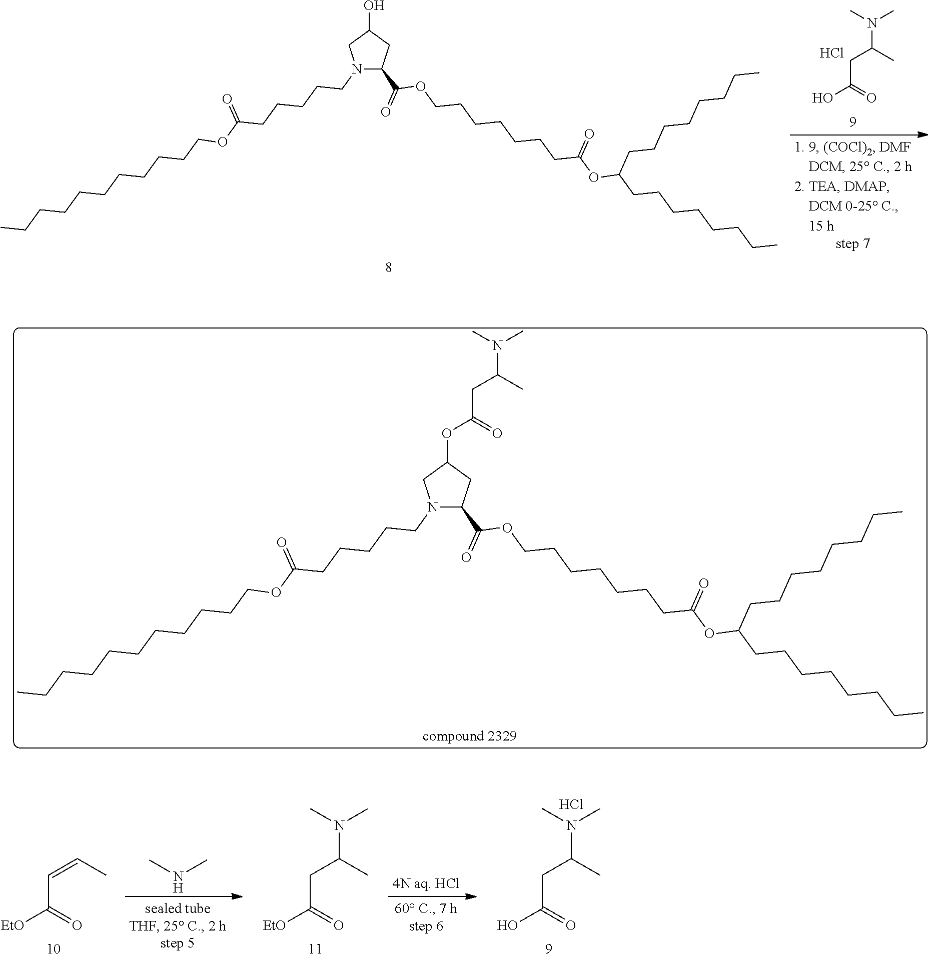

### Step 1

To a solution of 8-bromooctanoic acid (5 g, 22.41 mmol, 1.2 eq) in DCM (500 mL) was added EDCI (5.37 g, 28.01 mmol, 1.5 eq), DMAP (456.31 mg, 3.74 mmol, 0.2 eq), heptadecan-9-ol (4.79 g, 18.68 mmol, 1 eq) at 25° C. The mixture was degassed and purged with N₂ for 3 times, and then stirred at 25° C. for 8 hours under N₂ atmosphere. The reaction mixture was concentrated under reduced pressure to give a residue. The residue diluted with 500 mL H₂O, and then extracted with 800 mL EtOAc (400 mL×2). The combined organic layers were washed with 500 mL brine, dried over Na₂SO₄, filtered, and concentrated under reduced pressure to give a residue. The residue was purified by column chromatography (SiO₂, petroleum ether/ethyl acetate=1/0 to 10/1) to give 1-octylnonyl 8-bromooctanoate (24 g, 52.00 mmol, 92.81% yield) as colorless oil.

### Step 2

To a solution of (2S)-1-tert-butoxycarbonyl-4-hydroxy-pyrrolidine-2-carboxylic acid (2.5 g, 10.81 mmol, 1 eq) and Cs₂CO₃ (7.75 g, 23.78 mmol, 2.2 eq) in DMF (30 mL) was added 1-octylnonyl 8-bromooctanoate (5.99 g, 12.97 mmol, 1.2 eq) at 25° C. under N₂ atmosphere. The mixture was then stirred at 25° C. for 8 hours under N₂ atmosphere. The reaction mixture was diluted with 100 mL H₂O and extracted with 60 mL EtOAc (20 mL×3). Then the combined organic layers was washed with 90 mL brine (30 mL×3). The combined organic layers were dried over Na₂SO₄, filtered, and concentrated under reduced pressure to give a residue. The residue was purified by column chromatography (SiO₂, petroleum ether/ethyl acetate=1/0 to 0/1) to give O1-tert-butyl O2-[8-(1-octylnonoxy)-8-oxo-octyl](2S)-4-hydroxypyrrolidine-1,2-dicarboxylate (10.78 g, 17.62 mmol, 81.48% yield) as colorless oil.

### Step 3

A solution of TFA (465.86 mg, 4.09 mmol, 303.49 μL, 1 eq) in DCM (12 mL) was added to O1-tert-butyl O2-[8-(1-octylnonoxy)-8-oxo-octyl](2S)-4-hydroxypyrrolidine-1,2-dicarboxylate (2.5 g, 4.09 mmol, 1 eq) and purged with N₂ for 3 times, and then the mixture was stirred at 25° C. for 2 hours under N₂ atmosphere. The reaction mixture was concentrated under reduced pressure to get a residue. Then the residue was dissolved with EtOAc (30 mL), and the organic layer was washed with 60 mL saturated NaHCO₃ (20 mL×3) and 60 mL brine (20 mL×3), dried over Na₂SO₄, filtered, and concentrated under reduced pressure to give a residue. The residue was purified by column chromatography (SiO₂, petroleum ether/ethyl acetate=10/0 to 1/0, 3% NH₃·H₂O) to give [8-(1-octylnonoxy)-8-oxo-octyl](2S)-4-hydroxypyrrolidine-2-carboxylate (3.14 g, 6.14 mmol, 75.09% yield) as yellow oil.

### Step 4

The solution of [8-(1-octylnonoxy)-8-oxo-octyl](2S)-4-hydroxypyrrolidine-2-carboxylate (3.1 g, 6.06 mmol, 1 eq) and undecyl 6-oxohexanoate (2.07 g, 7.27 mmol, 1.2 eq) in DCM (30 mL) was stirred for 30 minutes at 25° C. and then NaBH(OAc)₃ (3.85 g, 18.17 mmol, 3 eq) was added. The mixture was degassed and purged with N₂ for 3 times, and then stirred at 25° C. for 7.5 hours under N₂ atmosphere. The reaction mixture was diluted with 50 mL H₂O and extracted with 60 mL DCM (20 mL×3). Then the combined organic layers was washed with 90 mL brine (30 mL×3), dried over Na₂SO₄, filtered, and concentrated under reduced pressure to give a residue. The residue was purified by column chromatography (SiO₂, petroleum ether/ethyl acetate=1/0 to 1:1) to give [8-(1-octylnonoxy)-8-oxo-octyl](2S)-4-hydroxy-1-(6-oxo-6-undecoxy-hexyl)pyrrolidine-2-carboxylate (2.86 g, 3.67 mmol, 60.52% yield) as yellow oil.

¹H NMR (400 MHz, CDCl₃), 4.84-4.90 (m, 1H), 4.25-4.55 (m, 1H), 4.01-4.20 (m, 5H), 3.40-3.60 (m, 1H), 3.06-3.32 (m, 1H), 2.40-2.71 (m, 3H), 2.15-2.40 (m, 5H), 1.90-2.05 (m, 1H), 1.55-1.80 (m, 11H), 1.15-1.40 (m, 51H), 0.89 (t, J=6.8 Hz, 9H).

### Step 5

A solution of ethyl (Z)-but-2-enoate (1 g, 8.76 mmol, 1 eq) in N-methylmethanamine (2 M, 20 mL, 4.57 eq, THF) was stirred at 25° C. for 2 hours in a 100 mL of sealed tube. The reaction mixture was concentrated under reduced pressure to get a residue. The residue was purified by column chromatography (SiO₂, petroleum ether/ethyl acetate=1/0 to 1/0) to give ethyl 3-(dimethylamino)butanoate (0.64 g, 4.02 mmol, 45.88% yield) as colorless oil.

### Step 6

Ethyl 3-(dimethylamino)butanoate (0.32 g, 2.01 mmol, 1 eq) was dissolved in aqeuous HCl (4 M, 20.98 mL, 41.76 eq), and stirred for 7 hours at 60° C. under N₂ atmosphere. The reaction mixture was diluted with 20 mL water and extracted with 60 mL EtOAc (20 mL×3), and then the aqueous phase was freeze-dried to give 3-(dimethylamino)butanoic acid (0.231 g, 1.76 mmol, 87.63% yield, HCl) as white solid.

### Step 7

A mixture of 3-(dimethylamino)butanoic acid (0.231 g, 1.76 mmol, 231.00 μL, 1 eq) in DCM (20 mL) was added (COCl)₂ (1.12 g, 8.81 mmol, 770.77 μL, 5 eq) and DMF (6.44 mg, 88.05 μmol, 6.77 μL, 0.05 eq) at 25° C. under N₂ atmosphere. The mixture was degassed and purged with N₂ for 3 times, and then stirred at 25° C. for 2 hours under N₂ atmosphere. The reaction mixture was concentrated under reduced pressure to give 3-(dimethylamino)butanoyl chloride (0.24 g, 1.60 mmol, 91.09% yield) as a yellow solid. To a solution of [8-(1-octylnonoxy)-8-oxo-octyl](2S)-4-hydroxy-1-(6-oxo-6-undecoxy-hexyl)pyrrolidine-2-carboxylate (0.25 g, 320.43 mol, 1 eq), TEA (226.97 mg, 2.24 mmol, 312.20 μL, 7 eq) and DMAP (19.57 mg, 160.21 mol, 0.5 eq) in DCM (25 mL) was added dropwise a solution of 3-(dimethylamino)butanoyl chloride (239.71 mg, 1.60 mmol, 5 eq) in DCM (5 mL) at 0° C. The mixture was stirred at 25° C. for 15 hours. The mixture was added in 30 mL saturated NaHCO₃, and then extracted with 150 mL DCM (50 mL×3). The combined organic layer was washed with brine (50 mL×3), dried over Na₂SO₄, filtered, and concentrated under reduced pressure to give a residue. The residue was purified by column chromatography (SiO₂, petroleum ether/ethyl acetate=10/1 to 1/0, 3% NH₃·H₂O) and prep-HPLC (column: Xselect CSH C18 100×30 mm×5 m; mobile phase: [H₂O(0.04% HCl)-ACN:THF=1:1]; gradient:40%-800% B over 8.0 minutes) to give [8-(1-octylnonoxy)-8-oxo-octyl](2S)-4-[3-(dimethylamino) butanoyloxy]-1-(6-oxo-6-undecoxy-hexyl)pyrrolidine-2-carboxylate (47 mg, 51.30 μmol, 47.70% yield, HCl) as white solid.

1H NMR (400 MHz, CDCl₃), 11.43-13.32 (m, 2H), 5.23-5.55 (m, 1H), 4.80-4.95 (m, 1H), 4.40-4.70 (m, 1H), 3.90-4.40 (m, 6H), 3.50-3.90 (m, 2H), 2.60-3.40 (m, 10H), 2.50-2.60 (m, 1H), 2.20-2.40 (m, 4H), 1.60-2.02 (m, 9H), 1.20-1.50 (m, 56H), 0.87 (t, J=6.8 Hz, 9H).

LCMS: (M+H⁺): 893.7 @11.259 minutes.

### 8.18. Synthesis of Compound 2336

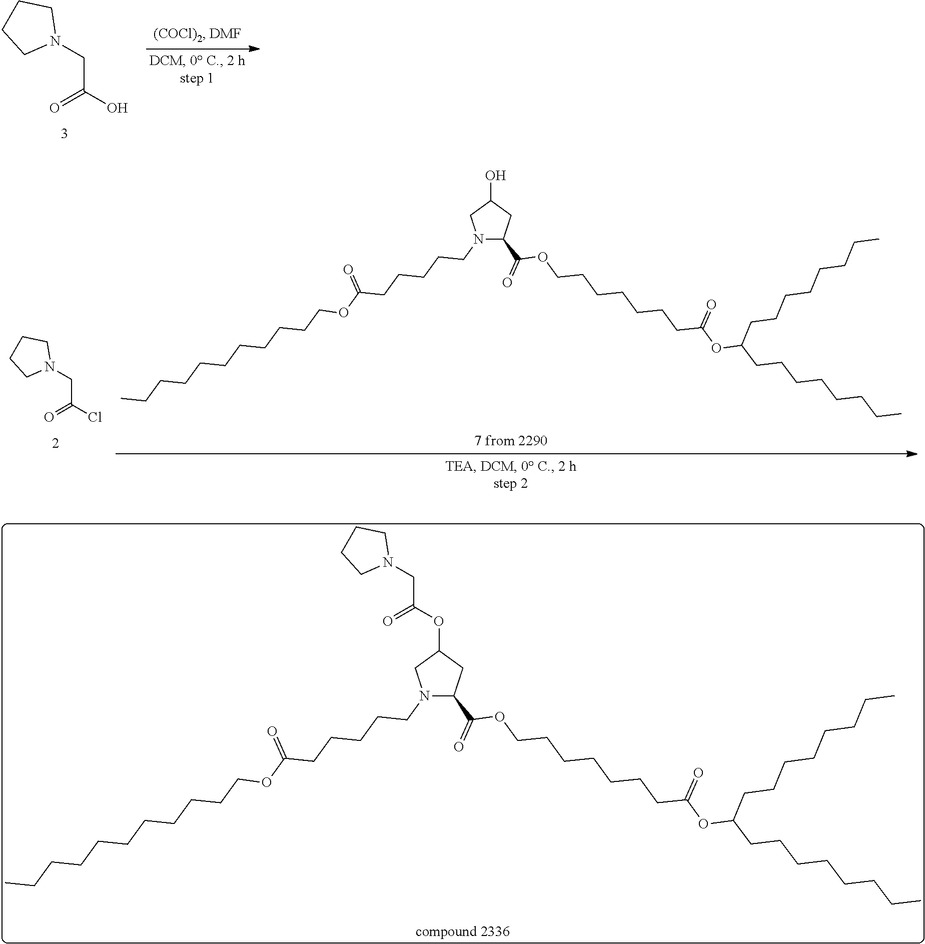

### Step 1

To a solution of 2-pyrrolidin-1-ylacetic acid (500 mg, 3.87 mmol, 1 eq) in DCM (10 mL) was added DMF (28.30 mg, 387.13 mol, 29.79 μL, 0.1 eq) and (COCl)₂ (589.64 mg, 4.65 mmol, 406.65 μL, 1.2 eq). The mixture was stirred at 0° C. for 2 hours. The mixture was concentrated under reduced pressure to give 2-pyrrolidin-1-ylacetyl chloride (570 mg, crude) as a white solid.

### Step 2

To a solution of [8-(1-octylnonoxy)-8-oxo-octyl](2S)-4-hydroxy-1-(6-oxo-6-undecoxy-hexyl) pyrrolidine-2-carboxylate (600 mg, 769.03 mol, 1 eq) in DCM (10 mL) was added TEA (778.17 mg, 7.69 mmol, 1.07 mL, 10 eq) and 2-pyrrolidin-1-ylacetyl chloride (567.55 mg, 3.85 mmol, 5 eq). The mixture was stirred at 0° C. for 2 hours. The mixture was concentrated under reduced pressure to give a residue. The residue was purified by prep-HPLC (column: Phenomenex Luna C18 100×30 mm×5 m; mobile phase: [water(HCl)-ACN]; B %: 45%-75%, 10 minutes). Then the mixture was adjusted to pH=8 with saturated NaHCO₃, and extracted with 30 mL EtOAc (10 mL×3). The combined organic layers were washed with saturated 30 mL brine (10 mL×3), dried over Na₂SO₄, filtered, and concentrated under reduced pressure to give [8-(1-octylnonoxy)-8-oxo-octyl](2S)-1-(6-oxo-6-undecoxy-hexyl)-4-(2-pyrrolidin-1-ylacetyl)oxy-pyrrolidine-2-carboxylate (127 mg, 142.48 mol, 18.53% yield) as yellow oil.

1H NMR (400 MHz, CDCl₃), 5.22-5.29 (m, 1H), 4.84-4.90 (m, 1H), 4.04-4.14 (m, 4H), 3.12-3.55 (m, 4H), 2.13-2.70 (m, 13H), 1.83 (d, J=3.6 Hz, 4H), 1.61-1.65 (m, 8H), 1.45-1.55 (m, 6H), 1.26-1.34 (m, 48H), 0.88 (t, J=6.4 Hz, 9H). (M+H⁺): 891.7.

LCMS: (M+H⁺): 891.7 @10.183&10.335 minutes.

### 8.19. Synthesis of Compound 2337

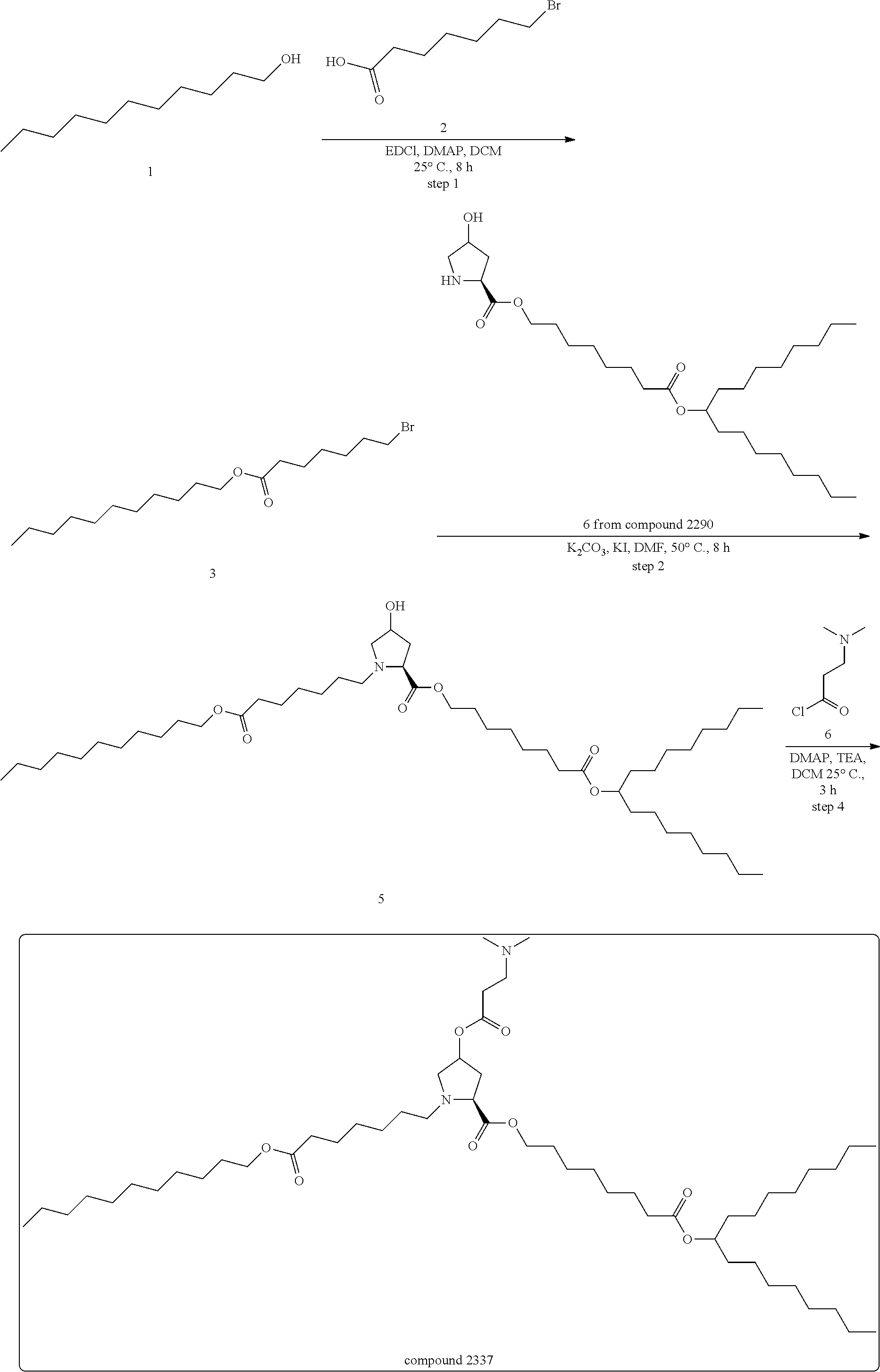

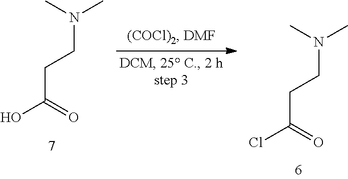

### Step 1

To a solution of undecan-1-ol (5 g, 29.02 mmol, 1 eq) and 7-bromoheptanoic acid (6.07 g, 29.02 mmol, 1 eq) in DCM (100 mL) was added DMAP (1.77 g, 14.51 mmol, 0.5 eq) and EDCI (6.68 g, 34.82 mmol, 1.2 eq). The mixture was stirred at 25° C. for 8 hours. The mixture was added into H₂O (200 mL), and extracted with EtOAc (200 mL×3). The organic layer was washed with brine (200 mL×2), dried over Na₂SO₄, filtered, and concentrated under reduced pressure. The residue was purified by column chromatography (SiO₂, petroleum ether/ethyl acetate=1/0 to 5/1) to give undecyl 7-bromoheptanoate (7 g, 19.26 mmol, 66.39% yield) as colorless oil.

### Step 2

To a solution of [8-(1-octylnonoxy)-8-oxo-octyl](2S)-4-hydroxypyrrolidine-2-carboxylate (0.5 g, 976.99 mol, 1 eq) and undecyl 7-bromoheptanoate (426.01 mg, 1.17 mmol, 1.2 eq) in DMF (15 mL) was added K₂CO₃ (405.09 mg, 2.93 mmol, 3 eq) and KI (81.09 mg, 488.50 μmol, 0.5 eq). The mixture was stirred at 50° C. for 8 hours. The reaction mixture was quenched by addition H₂O 20 mL at 25° C., and then extracted with EtOAc (20 mL×3). The combined organic layers were washed with brine (20 mL×2), dried over Na₂SO₄, filtered, and concentrated under reduced pressure to give a residue. The residue was purified by column chromatography (SiO₂, petroleum ether/ethyl acetate=20/1 to 0/1) to give [8-(1-octylnonoxy)-8-oxo-octyl](2S)-4-hydroxy-1-(7-oxo-7-undecoxy-heptyl)pyrrolidine-2-carboxylate (700 mg, 881.35 mol, 45.11% yield) as a yellow oil.

¹H NMR (400 MHz, CDCl₃), 4.82-4.92 (m, 1H), 4.03-4.16 (m, 6H), 2.22-2.37 (m, 6H), 1.58-1.67 (m, 12H), 1.47-1.53 (m, 4H), 1.24-1.37 (m, 52H), 0.85-0.92 (m, 9H).

### Step 3

To a solution of 3-(dimethylamino)propanoic acid (200 mg, 1.71 mmol, 1 eq) in DCM (5 mL) was added (COCl)₂ (1.08 g, 8.54 mmol, 747.26 μL, 5 eq) and DMF (12.48 mg, 170.73 μmol, 13.14 μL, 0.1 eq). The mixture was stirred at 25° C. for 2 hours. The reaction mixture was concentrated under reduced pressure to give 3-(dimethylamino)propanoyl chloride (293.7 mg, 1.71 mmol, 99.99% yield, HCl) as a yellow solid.

### Step 4

To the suspension of [8-(1-octylnonoxy)-8-oxo-octyl](2S)-4-hydroxy-1-(7-oxo-7-undecoxy-heptyl)pyrrolidine-2-carboxylate (400 mg, 503.63 mol, 1 eq), TEA (254.81 mg, 2.52 mmol, 350.50 μL, 5 eq) and DMAP (30.76 mg, 251.81 mol, 0.5 eq) in DCM (3 mL) was added dropwise 3-(dimethylamino)propanoyl chloride (259.95 mg, 1.51 mmol, 3 eq, HCl) in DCM (1 mL) at 25° C. The mixture was stirred at 25° C. for 3 hours under N₂ atmosphere. The reaction mixture was quenched by addition of 10 mL aqueous NaHCO₃ at 25° C., and then extracted with 30 mL EtOAc (10 mL×3). The combined organic layers were washed with 20 mL brine (10 mL×2), dried over Na₂SO₄, filtered and concentrated under reduced pressure to give a residue. The residue was purified by prep-HPLC (column: Phenomenex Luna C18 100×30 mm×5 μm; mobile phase: [water(HCl)-ACN]; B %: 40%-70%, 10 minutes) to give the solution. The solution was added saturated NaHCO₃ until the solution has pH=˜7, and then extracted with EtOAc (20 mL×3). The organic layer was washed brine (20 mL×2), dried over Na₂SO₄, filtered, and concentrated under reduced pressure to give [8-(1-octylnonoxy)-8-oxo-octyl](2S)-4-[3-(dimethylamino)propanoyloxy]-1-(7-oxo-7-undecoxy-heptyl)pyrrolidine-2-carboxylate (60 mg, 156.71 mol, 13.34% yield) as a colorless oil.

¹H NMR (400 MHz, CDCl₃), 5.17-5.32 (m, 1H), 4.80-4.93 (m, 1H), 4.02-4.17 (m, 4H), 3.06-3.58 (m, 2H), 1.91-2.85 (m, 19H), 1.60 (s, 8H), 1.48-1.54 (m, 6H), 1.24-1.36 (m, 50H), 0.85-0.91 (m, 9H). LCMS: (M+H⁺): 893.7 @10.113/10.172 minutes.

### 8.20. Synthesis of Compound 2338

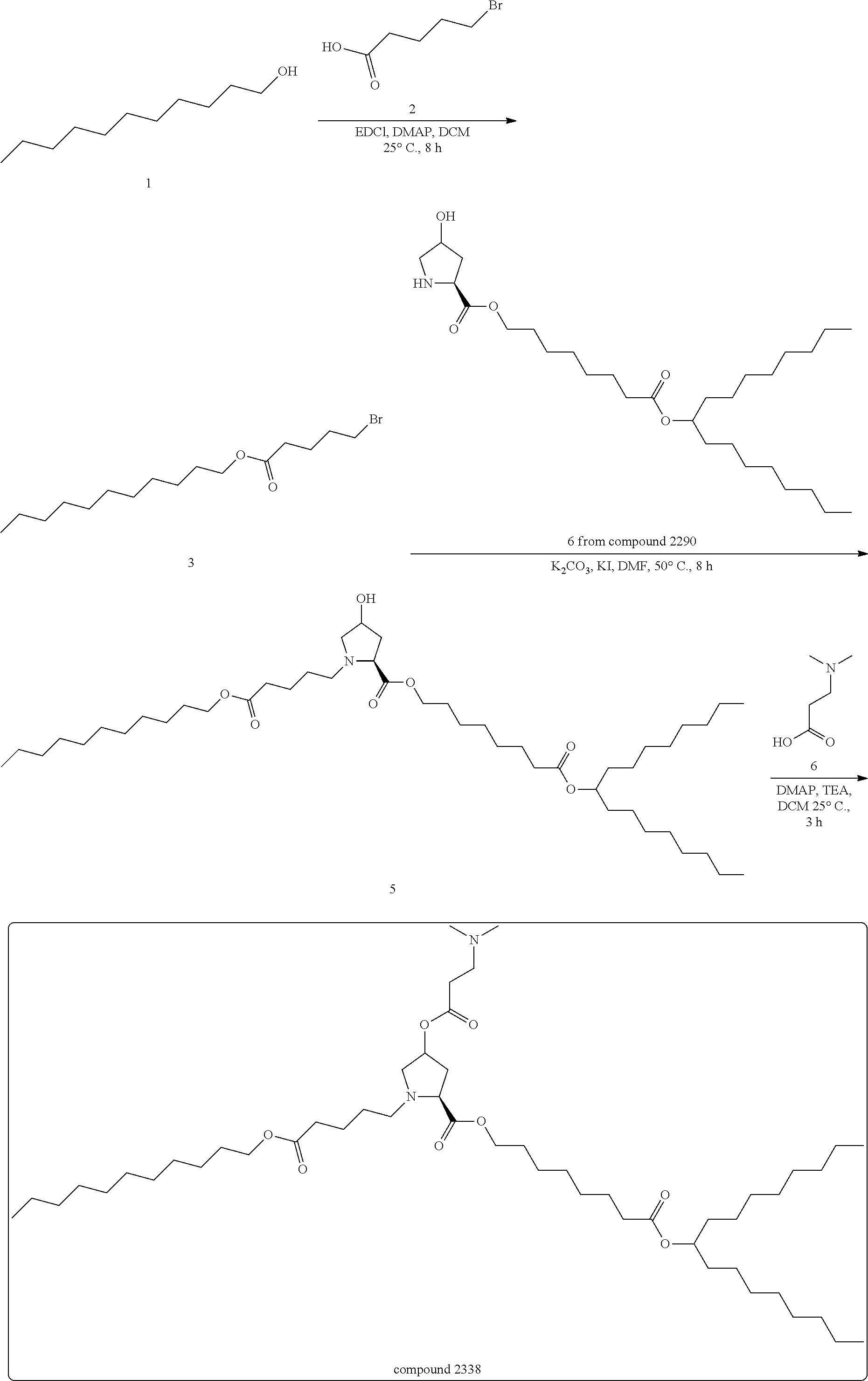

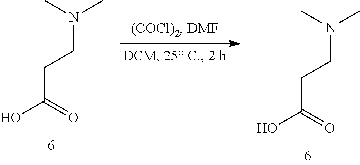

### Step 1

To a solution of undecan-1-ol (5 g, 29.02 mmol, 1 eq) and 5-bromopentanoic acid (5.25 g, 29.02 mmol, 1 eq) in DCM (100 mL) was added DMAP (1.77 g, 14.51 mmol, 0.5 eq) and EDCI (6.68 g, 34.82 mmol, 1.2 eq). The mixture was stirred at 25° C. for 8 hours. The mixture was added into H₂O (200 mL), and extracted with EtOAc (100 mL×3). The organic layer was washed with brine (100 mL×2), dried over Na₂SO₄, filtered, and concentrated under reduced pressure. The residue was purified by column chromatography (SiO₂, petroleum ether/ethyl acetate=1/0 to 5/1) to give undecyl 5-bromopentanoate (7 g, 20.88 mmol, 71.94% yield) as colorless oil.

### Step 2

To a solution of [8-(1-octylnonoxy)-8-oxo-octyl](2S)-4-hydroxypyrrolidine-2-carboxylate (0.5 g, 976.99 mol, 1 eq) and undecyl 5-bromopentanoate (393.12 mg, 1.17 mmol, 1.2 eq) in DMF (15 mL) was added K₂CO₃ (405.09 mg, 2.93 mmol, 3 eq) and KI (81.09 mg, 488.50 μmol, 0.5 eq). The mixture was stirred at 50° C. for 8 hours. The reaction mixture was quenched by addition of 20 mL H₂O at 15° C., and then extracted with 60 mL EtOAc (20 mL×3). The combined organic layers were washed with 40 mL brine (20 mL×2), dried over Na₂SO₄, filtered, and concentrated under reduced pressure to give a residue. The residue was purified by column chromatography (SiO₂, petroleum ether/ethyl acetate=1/0 to 0/1) to give [8-(1-octylnonoxy)-8-oxo-octyl](2S)-4-hydroxy-1-(5-oxo-S-undecoxy-pentyl)pyrrolidine-2-carboxylate (700 mg, 913.62 mol, 46.76% yield) as yellow oil.

### Step 3

To a solution of 3-(dimethylamino)propanoic acid (200 mg, 1.71 mmol, 1 eq) in DCM (5 mL) was added (COCl)₂ (1.08 g, 8.54 mmol, 747.26 μL, 5 eq) and DMF (12.48 mg, 170.73 μmol, 13.14 μL, 0.1 eq). The mixture was stirred at 25° C. for 2 hours. The reaction mixture was concentrated under reduced pressure to give 3-(dimethylamino)propanoyl chloride (293.7 mg, 1.71 mmol, 99.99% yield, HCl) as a yellow solid.

### Step 4

To the suspension of 3-(dimethylamino)propanoyl chloride (269.47 mg, 1.57 mmol, 3 eq, HCl), TEA (264.14 mg, 2.61 mmol, 363.33 μL, 5 eq) and DMAP (31.89 mg, 261.03 mol, 0.5 eq) in DCM (5 mL) was added dropwise [8-(1-octylnonoxy)-8-oxo-octyl](2S)-4-hydroxy-1-(5-oxo-S-undecoxy-pentyl)pyrrolidine-2-carboxylate (400 mg, 522.07 mol, 1 eq) in DCM (3 mL) at 25° C. The mixture was stirred at 25° C. for 3 hours under N₂ atmosphere. The reaction mixture was quenched by addition of 10 mL aqueous NaHCO₃ at 25° C., and then extracted with 30 mL EtOAc (10 mL×3). The combined organic layers were washed with 20 mL brine (10 mL×2), dried over Na₂SO₄, filtered, and concentrated under reduced pressure to give a residue. The residue was purified by prep-HPLC (column: Phenomenex Luna C18 100×30 mm×5 m; mobile phase: [water(HCl)-ACN]; B %: 40%-70%, 10 minutes) to give a solution. The solution was added into saturated NaHCO₃ (100 mL), and extracted with EtOAc (20 mL×3). The organic layer was washed with brine (20 mL×2), dried over Na₂SO₄, filtered, and the filtrate was concentrated under reduced pressure to give [8-(1-octylnonoxy)-8-oxo-octyl](2S)-4-[3-(dimethylamino)propanoyloxy]-1-(5-oxo-S-undecoxy-pentyl)pyrrolidine-2-carboxylate (110 mg, 161.79 mol, 24.35% yield) as colorless oil.

¹H NMR (400 MHz, CDCl₃), 5.14-5.32 (m, 1H), 4.81-4.95 (m, 1H), 3.99-4.19 (m, 4H), 3.06-3.60 (m, 2H), 2.41-2.83 (m, 7H), 1.99-2.35 (m, 12H), 1.59-1.67 (m, 8H), 1.47-1.56 (m, 6H), 1.23-1.37 (m, 46H), 0.82-0.94 (m, 9H. LCMS: (M+H⁺): 865.6 @11.572 minutes.

### 8.21. Synthesis of Compound 2339

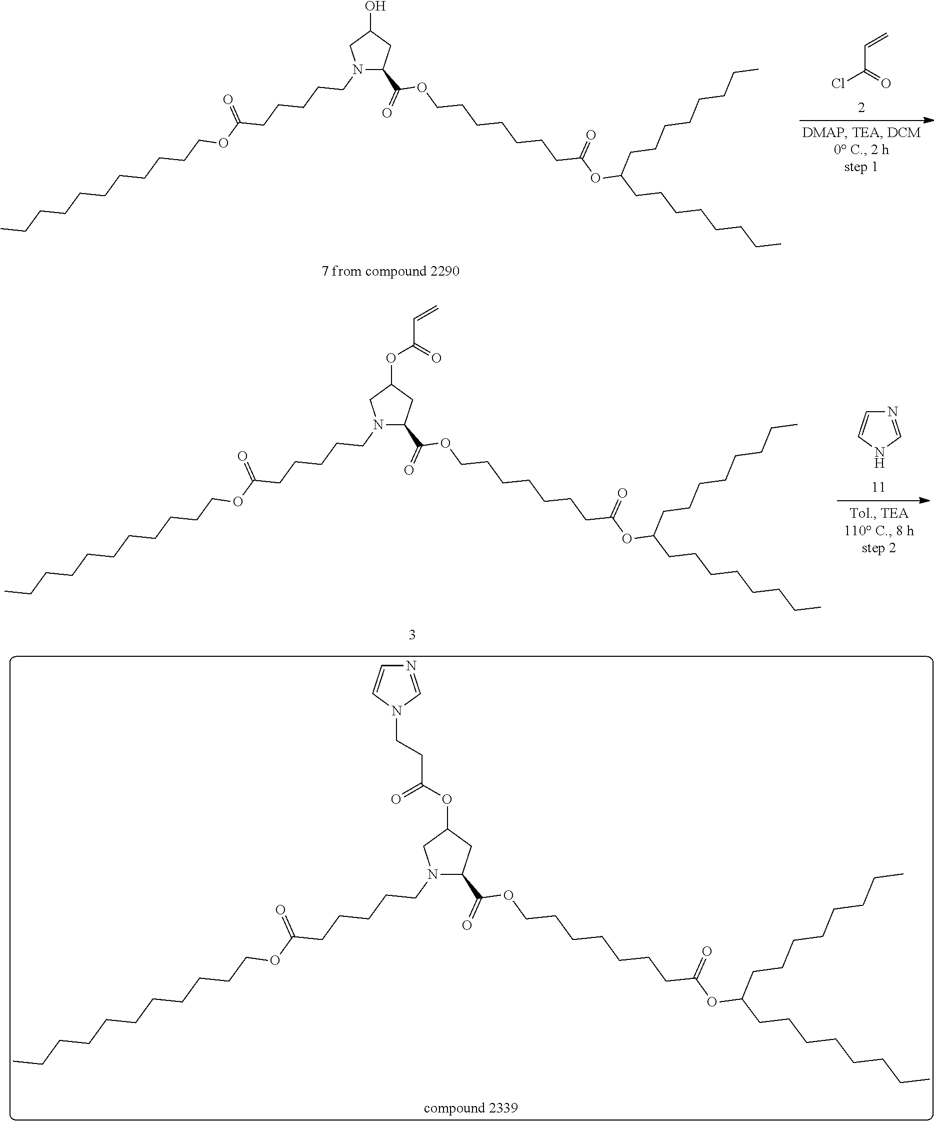

### Step 1

To a solution of [8-(1-octylnonoxy)-8-oxo-octyl](2S)-4-hydroxy-1-(6-oxo-6-undecoxy-hexyl) pyrrolidine-2-carboxylate (1 g, 1.28 mmol, 1 eq), TEA (648.47 mg, 6.41 mmol, 891.99 μL, 5 eq) and DMAP (78.29 mg, 640.85 mol, 0.5 eq) in DCM (10 mL) was added dropwise prop-2-enoyl chloride (464.02 mg, 5.13 mmol, 418.04 μL, 4 eq) at 0° C., and then the mixture was stirred at 0° C. for 2 hours under N₂ atmosphere. The reaction mixture was concentrated under reduced pressure to give a residue. The residue was purified by column chromatography (SiO₂, petroleum ether/ethyl acetate=1/0 to 3/1) to give [8-(1-octylnonoxy)-8-oxo-octyl](2S)-1-(6-oxo-6-undecoxy-hexyl)-4-prop-2-enoyloxy-pyrrolidine-2-carboxylate (0.45 g, 539.40 μmol, 42.08% yield) as colorless oil.

¹H NMR (400 MHz, CDCl₃), 6.42 (d, J=11.6 Hz, 1H), 6.08-6.18 (m, 1H), 5.58 (t, J=10.0 Hz, 1H), 5.21-5.48 (m, 1H), 4.86-4.88 (m, 1H), 4.04-4.15 (m, 5H), 3.15-3.75 (m, 2H), 2.03-2.89 (m, 9H), 1.50-1.66 (m, 17H), 1.27-1.30 (m, 44H), 0.87 (t, J=5.6 Hz, 9H).

### Step 2

A mixture of [8-(1-octylnonoxy)-8-oxo-octyl](2S)-1-(6-oxo-6-undecoxy-hexyl)-4-prop-2-enoyloxy-pyrrolidine-2-carboxylate (200 mg, 239.73 mol, 1 eq), imidazole (48.96 mg, 719.20 mol, 3 eq), TEA (24.26 mg, 239.73 mol, 33.37 μL, 1 eq) in Tol. (5 mL) was degassed and purged with N₂ for 3 times, and then the mixture was stirred at 110° C. for 8 hours under N₂ atmosphere. The reaction mixture was concentrated under reduced pressure to give a residue. The residue was purified by prep-TLC (SiO₂, petroleum ether/ethyl acetate=0:1, 0.3% NH₃·H₂O) and prep-HPLC (column: Phenomenex Luna C18 100×30 mm×5 m; mobile phase: [water(HCl)-ACN]; B %: 45%-75%, 10 minutes). The mixture was concentrated under reduced pressure and then washed with saturated 30 mL NaHCO₃ and extracted with 60 mL EtOAc (30 mL×2). The combined organic layers were washed with 20 mL brine, dried over Na₂SO₄, filtered, and concentrated under reduced pressure to give [8-(1-octylnonoxy)-8-oxo-octyl](2S)-4-(3-imidazol-1-ylpropanoyloxy)-1-(6-oxo-6-undecoxy-hexyl)pyrrolidine-2-carboxylate (45 mg, 49.87 mol, 37.50% yield) as colorless oil.

1H NMR (400 MHz, CDCl₃), 7.62 (d, J=27.2 Hz, 1H), 7.08 (s, 1H), 7.96 (d, J=27.2 Hz, 1H), 5.19-5.27 (m, 1H), 4.85-4.89 (m, 1H), 4.28 (t, J=6.8 Hz, 2H), 4.04-4.12 (m, 4H), 3.44-3.48 (m, 1H), 3.01-3.33 (m, 1H), 2.77-2.80 (m, 2H), 2.48-2.71 (m, 3H), 2.28-2.31 (m, 5H), 2.01-2.21 (m, 1H), 1.50-1.62 (m, 14H), 1.27-1.45 (m, 48H), 0.89 (t, J=6.8 Hz, 9H).

LCMS: (M+H⁺): 902.6 @11.521 minutes.

### 8.22. Synthesis of Compound 2340

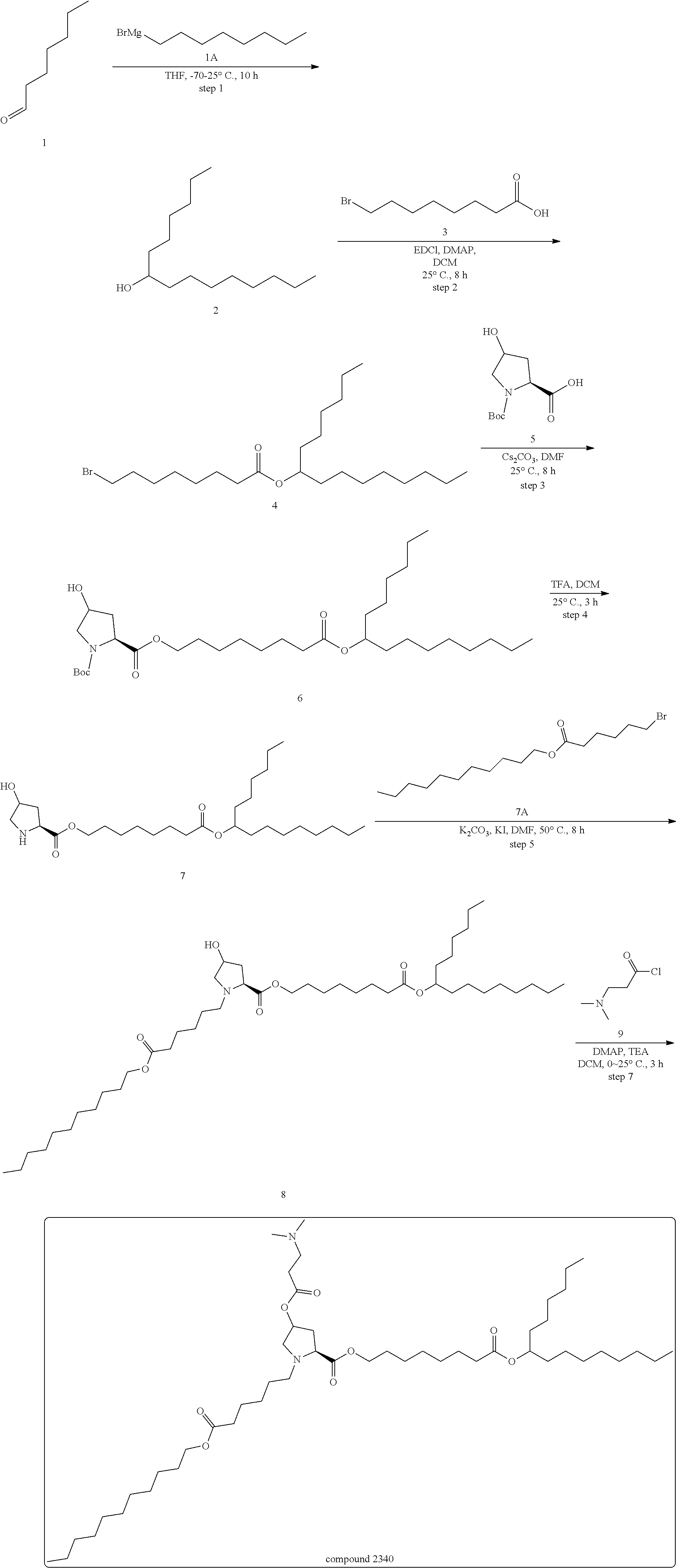

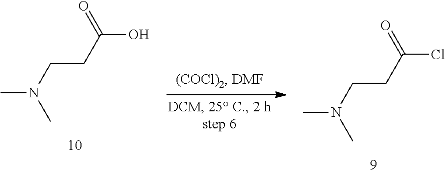

### Step 1

To a solution of heptanal (20 g, 175.15 mmol, 24.45 mL, 1 eq) in THF (200 mL) was added bromo(octyl)magnesium (2 M, 96.33 mL, 1.1 eq) at −70° C. The mixture was stirred at 25° C. for 10 hours. The reaction mixture (5 batches were combined) was quenched by addition of 300 mL saturated NH₄C₁ at 0° C., then diluted with 2000 mL H₂O, and extracted with EtOAc (500 mL×3). The combined organic layers were washed with 500 mL saturated NaCl, dried over Na₂SO₄, filtered, and concentrated under reduced pressure to give a residue. The residue was purified by column chromatography (SiO₂, petroleum ether/ethyl acetate=1/0 to 50/1) to give pentadecan-7-ol (20 g, 87.56 mmol, 25.00% yield) as a white solid.

¹H NMR (400 MHz, CDCl₃), 3.49-3.56 (m, 1H), 1.15-1.36 (m, 24H), 0.79-0.82 (m, 6H).

### Step 2

To a solution of pentadecan-7-ol (5 g, 21.89 mmol, 1 eq) and 8-bromooctanoic acid (4.88 g, 21.89 mmol, 1 eq) in DCM (50 mL) was added EDCI (5.04 g, 26.27 mmol, 1.2 eq) and DMAP (1.34 g, 10.95 mmol, 0.5 eq). The mixture was stirred at 25° C. for 8 hours. The reaction mixture diluted with water 50 mL and extracted with EtOAc 60 mL (20 mL×3). The combined organic layers were washed with 30 mL saturated brine (10 mL×3), dried over Na₂SO₄, filtered, and concentrated under reduced pressure to give a residue. The residue was purified by column chromatography (SiO₂, petroleum ether/ethyl acetate=1/0) to give 1-hexylnonyl 8-bromooctanoate (6.5 g, 14.99 mmol, 68.50% yield) as a colorless oil.

¹H NMR (400 MHz, CDCl₃), 4.84-4.91 (m, 1H), 3.41 (t, J=6.8 Hz, 2H), 2.29 (t, J=7.6 Hz, 2H), 1.82-1.89 (m, 2H), 1.59-1.67 (m, 2H), 1.40-1.52 (m, 6H), 1.26-1.36 (m, 24H), 0.88 (t, J=6.4 Hz, 6H).

### Step 3

To a solution of 1-hexylnonyl 8-bromooctanoate (3 g, 6.92 mmol, 1.2 eq) in DMF (30 mL) was added Cs₂CO₃ (4.13 g, 12.69 mmol, 2.2 eq) and (2S)-1-tert-butoxycarbonyl-4-hydroxy-pyrrolidine-2-carboxylic acid (1.33 g, 5.77 mmol, 1 eq). The mixture was stirred at 25° C. for 8 hours. The reaction mixture was diluted with 50 mL water and extracted with 60 mL EtOAc (20 mL×3). The combined organic layers were washed with 30 mL saturated brine (10 mL×3), dried over Na₂SO₄, filtered, and concentrated under reduced pressure to give a residue. The residue was purified by column chromatography (SiO₂, petroleum ether/ethyl acetate=20/1 to 3/1) to give O1-tert-butyl O2-[8-(1-hexylnonoxy)-8-oxo-octyl](2S)-4-hydroxypyrrolidine-1,2-dicarboxylate (2.7 g, 4.62 mmol, 80.19% yield) as yellow oil.

¹H NMR (400 MHz, CDCl₃), 4.86-4.89 (m, 1H), 4.28-4.52 (m, 2H), 4.11-4.25 (m, 3H), 3.46-3.69 (m, 2H), 2.29-2.40 (m, 3H), 2.06-2.11 (m, 2H), 1.26-1.53 (m, 41H), 0.89 (t, J=6.4 Hz, 6H).

### Step 4

To a solution of O1-tert-butyl O2-[8-(1-hexylnonoxy)-8-oxo-octyl](2S)-4-hydroxypyrrolidine-1,2-dicarboxylate (2 g, 3.43 mmol, 1 eq) in DCM (14 mL) was added TFA (10.78 g, 94.54 mmol, 7 mL, 27.60 eq). The mixture was stirred at 25° C. for 3 hours. The reaction mixture was concentrated under reduced pressure to remove solvent. The reaction mixture was adjusted to pH=8 with saturated NaHCO₃ and extracted with 30 mL EtOAc (10 mL×3). The combined organic layers were washed with 20 mL saturated brine (10 mL×2), dried over Na₂SO₄, filtered, and concentrated under reduced pressure to give [8-(1-hexylnonoxy)-8-oxo-octyl](2S)-4-hydroxypyrrolidine-2-carboxylate (1.5 g, crude) as colorless oil.

1H NMR (400 MHz, CDCl₃), 4.86-4.91 (m, 1H), 4.41-4.49 (m, 1H), 4.12-4.20 (m, 3H), 3.07-3.21 (m, 2H), 2.30 (t, J=7.6 Hz, 3H), 2.06-2.11 (m, 2H), 1.62-1.67 (m, 4H), 1.51-1.53 (m, 4H), 1.27-1.36 (m, 26H), 0.89 (t, J=6.4 Hz, 6H)

### Step 5

To a solution of [8-(1-hexylnonoxy)-8-oxo-octyl](2S)-4-hydroxypyrrolidine-2-carboxylate (1.5 g, 3.10 mmol, 1 eq) in DMF (30 mL) was added K₂CO₃ (1.29 g, 9.30 mmol, 3 eq) and KI (257.38 mg, 1.55 mmol, 0.5 eq) and undecyl 6-bromohexanoate (1.19 g, 3.41 mmol, 1.1 eq). The mixture was stirred at 50° C. for 8 hours. The reaction mixture was diluted with 50 mL water and extracted with 60 mL EtOAc (20 mL×3). The combined organic layers were washed with 30 mL saturated brine (15 mL×2), dried over Na₂SO₄, filtered, and concentrated under reduced pressure to give a residue. The residue was purified by column chromatography (SiO₂, petroleum ether/ethyl acetate=1/0 to 5/1) to give [8-(1-hexylnonoxy)-8-oxo-octyl](2S)-4-hydroxy-1-(6-oxo-6-undecoxy-hexyl) pyrroli dine-2-carboxylate (2 g, 2.66 mmol, 85.75% yield) as a colorless oil.

### Step 6

To a solution of 3-(dimethylamino)propanoic acid (500 mg, 3.26 mmol, 1 eq, HCl) in DCM (10 mL) was added DMF (23.79 mg, 325.51 mol, 25.04 μL, 0.1 eq) and (COCl)₂ (495.78 mg, 3.91 mmol, 341.92 μL, 1.2 eq). The mixture was stirred at 25° C. for 2 hours. The reaction mixture was concentrated under reduced pressure to give 3-(dimethylamino) propanoyl chloride (560 mg, crude, HCl) as a white solid.

### Step 7

To a solution of [8-(1-hexylnonoxy)-8-oxo-octyl](2S)-4-hydroxy-1-(6-oxo-6-undecoxy-hexyl)pyrrolidine-2-carboxylate (500 mg, 664.76 mol, 1 eq) in DCM (10 mL) was added DMAP (16.24 mg, 132.95 mol, 0.2 eq) and TEA (672.66 mg, 6.65 mmol, 925.25 μL, 10 eq) and 3-(dimethylamino)propanoyl chloride (554.71 mg, 3.22 mmol, 4.85 eq, HCl) at 0° C. The mixture was stirred at 25° C. for 3 hours. The reaction mixture was diluted with 40 mL water and extracted with 45 mL EtOAc (15 mL×3). The combined organic layers were washed with 30 mL brine (15 mL×2), dried over Na₂SO₄, filtered, and concentrated under reduced pressure to give a residue. The residue was purified by column chromatography (SiO₂, petroleum ether/ethyl acetate=0/1 to 1/1). Then the residue was purified by prep-HPLC (column: Phenomenex Luna C18 100×30 mm×5 m; mobile phase: [water(HCl)-ACN]; B %: 40%-70%, 10 minutes). Then the mixture was adjusted to pH=8 with saturated NaHCO₃, extracted with EtOAc 45 mL (15 mL×3). The combined organic layers were dried over Na₂SO₄, filtered, and concentrated under reduced pressure to give [8-(1-hexylnonoxy)-8-oxo-octyl](2S)-4-[3-(dimethylamino)propanoyloxy]-1-(6-oxo-6-undecoxy-hexyl)pyrrolidine-2-carboxylate (380 mg, 446.38 mol, 67.15% yield) as colorless oil.

¹H NMR (400 MHz, CDCl₃), 5.18-5.28 (m, 1H), 4.85-4.88 (m, 1H), 4.03-4.14 (m, 4H), 3.09-3.54 (m, 2H), 2.47-2.62 (m, 7H), 2.00-2.31 (m, 12H), 1.61-1.63 (m, 8H), 1.49-1.51 (m, 6H), 1.26-1.33 (m, 44H), 0.88 (t, J=6.8 Hz, 9H). LCMS: (M+H⁺): 851.8 @10.805 minutes.

### 8.23. Synthesis of Compound 2341

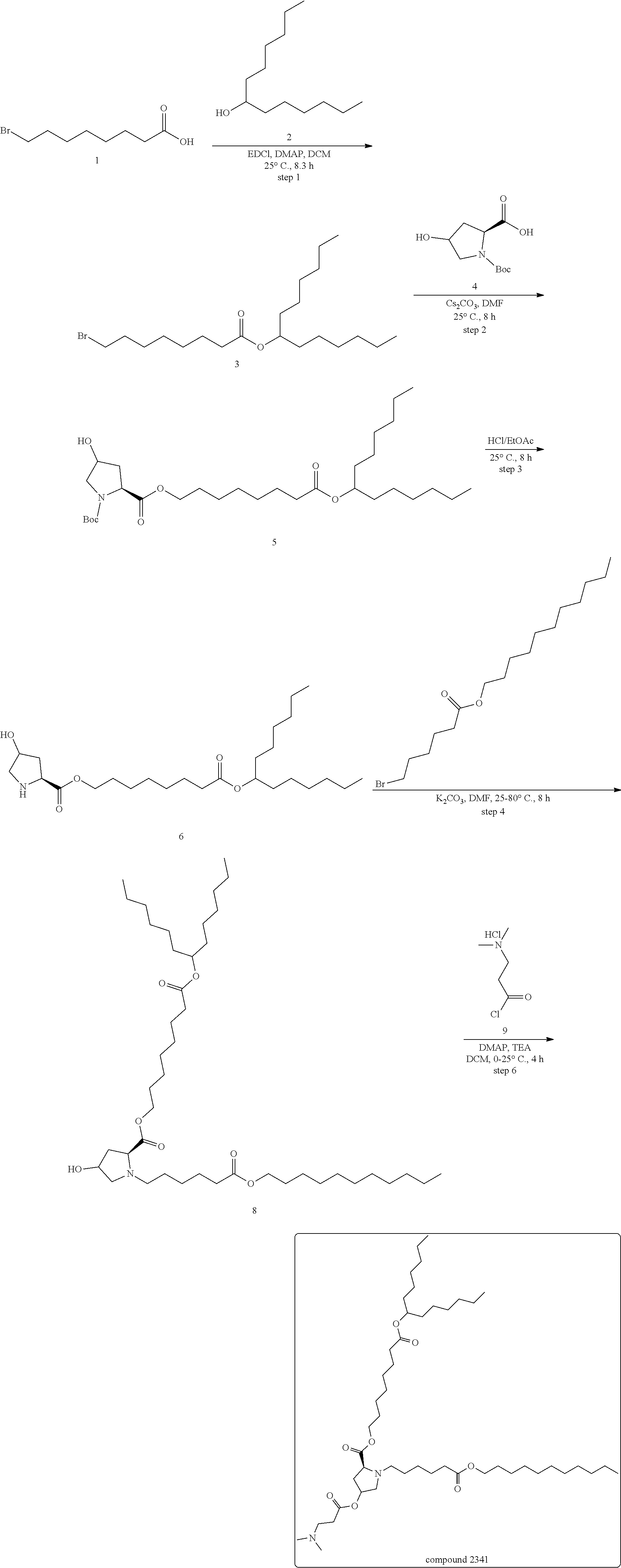

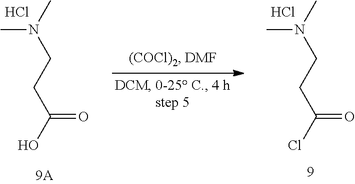

### Step 1

To a solution of 8-bromooctanoic acid (9.47 g, 42.42 mmol, 1.7 eq) in DCM (200 mL) was added EDCI (8.13 g, 42.42 mmol, 1.7 eq) and DMAP (3.35 g, 27.45 mmol, 1.1 eq). The mixture was degassed and purged with N₂ for 3 times, and then stirred at 25° C. for 0.3 hour under N₂ atmosphere. To the mixture was added tridecan-7-ol (5 g, 24.96 mmol, 1 eq) and the mixture was stirred at 25° C. for 8 hours under N₂ atmosphere. The reaction mixture was concentrated under reduced pressure, and then diluted with 200 mL H₂O and extracted with 900 mL EtOAc (300 mL×3). The combined organic layers were washed with 200 mL brine, dried over Na₂SO₄, filtered, and concentrated under reduced pressure to give a residue. The residue was purified by column chromatography (SiO₂, petroleum ether/ethyl acetate=1/0 to 10/1) to give 1-hexylheptyl 8-bromooctanoate (7.78 g, 19.18 mmol, 76.85% yield) as colorless oil.

1H NMR (400 MHz, CDCl₃), 4.85-4.88 (m, 1H), 3.41 (t, J=6.8 Hz, 2H), 2.29 (t, J=7.6 Hz, 2H), 1.84-1.90 (m, 2H), 1.59-1.67 (m, 2H), 1.40-1.57 (m, 8H), 1.25-1.38 (m, 18H), 0.88 (t, J=6.8 Hz, 6H).

### Step 2

To a solution of 1-hexylheptyl 8-bromooctanoate (7.28 g, 17.95 mmol, 1.2 eq) and (2S)-1-tert-butoxycarbonyl-4-hydroxy-pyrrolidine-2-carboxylic acid (3.46 g, 14.95 mmol, 1 eq) in DMF (200 mL) was added Cs₂CO₃ (10.72 g, 32.90 mmol, 2.2 eq) at 25° C. The mixture was degassed and purged with N₂ for 3 times, and then stirred for 8 hours under N₂ atmosphere. The reaction mixture was diluted with 100 mL H₂O, and extracted with 600 mL EtOAc (200 mL×3). The combined organic layers were washed with 300 mL brine (300 ml×2) mL, dried over Na₂SO₄, filtered, and concentrated under reduced pressure to give a residue. The residue was purified by column chromatography (SiO₂, petroleum ether/ethyl acetate=10/1 to 0/1, 5% NH₃·H₂O) to give O1-tert-butyl O2-[8-(1-hexylheptoxy)-8-oxo-octyl](2S)-4-hydroxypyrrolidine-1,2-dicarboxylate (6.31 g, 11.36 mmol, 75.97% yield) as yellow oil.

¹H NMR (400 MHz, CDCl₃), 4.85-4.88 (m, 1H), 4.05-4.35 (m, 2H), 4.35-4.55 (m, 2H), 3.38-3.75 (m, 2H), 2.20-2.44 (m, 3H), 2.25-2.17 (m, 1H), 1.59-1.67 (m, 4H), 1.40-1.55 (m, 13H), 1.20-1.38 (m, 22H), 0.88 (t, J=6.8 Hz, 6H).

### Step 3

To a solution of O1-tert-butyl O2-[8-(1-hexylheptoxy)-8-oxo-octyl](2S)-4-hydroxypyrrolidine-1,2-dicarboxylate (5.7 g, 10.26 mmol, 1 eq) in EtOAc (31.5 mL) was added HCl/EtOAc (4 M, 31.5 mL, 12.29 eq). The mixture was stirred at 25° C. for 8 hours. The reaction mixture was adjusted to pH=7.0 with aqueous saturated NaHCO₃ (60 mL) and extracted with 300 mL EtOAc (100 mL×3). The combined organic layers were dried over Na₂SO₄, filtered, and concentrated under reduced pressure to give [8-(1-hexylheptoxy)-8-oxo-octyl](2S)-4-hydroxypyrrolidine-2-carboxylate (4.39 g, 9.63 mmol, 93.94% yield) as yellow oil.

### Step 4

To a solution of [8-(1-hexylheptoxy)-8-oxo-octyl](2S)-4-hydroxypyrrolidine-2-carboxylate (4.36 g, 9.56 mmol, 1 eq) and undecyl 6-bromohexanoate (4.00 g, 11.48 mmol, 1.2 eq) in DMF (100 mL) was added K₂CO₃ (3.97 g, 28.72 mmol, 3 eq) at 25° C. The mixture was degassed and purged with N₂ for 3 times, and then stirred at 80° C. for 8 hours under N₂ atmosphere. The reaction mixture was filtered and diluted with 150 mL H₂O, and extracted with 400 mL EtOAc (100 mL×4). The combined organic layers were washed with 300 mL brine (150 ml×2), dried over Na₂SO₄, filtered, and concentrated under reduced pressure to give a residue. The residue was purified by column chromatography (SiO₂, petroleum ether/ethyl acetate=8/1 to 1/1, 5% NH₃·H₂O) to give[8-(1-hexylheptoxy)-8-oxo-octyl](2S)-4-hydroxy-1-(6-oxo-6-undecoxy-hexyl) pyrrolidine-2-carboxylate (4.21 g, 5.81 mmol, 60.77% yield) as yellow oil.

### Step 5

To a solution of 3-(dimethylamino)propanoic acid (600 mg, 3.91 mmol, 1 eq, HCl) in DCM (10 mL) was added oxalyl dichloride (2.48 g, 19.53 mmol, 1.71 mL, 5 eq) and DMF (19.00 mg, 259.94 mol, 0.02 mL, 6.65e-2 eq) at 0° C. The mixture was degassed and purged with N₂ for 3 times, and then warmed to 25° C. and stirred for 4 hours under N₂ atmosphere. The reaction mixture was concentrated under reduced pressure to give crude product 3-(dimethylamino)propanoyl chloride (700 mg, crude, HCl) as a yellow solid and used into the next step without further purification.

### Step 6

To a solution of [8-(1-hexylheptoxy)-8-oxo-octyl](2S)-4-hydroxy-1-(6-oxo-6-undecoxy-hexyl)pyrrolidine-2-carboxylate (500 mg, 690.51 mol, 1 eq) and DMAP (8.44 mg, 69.05 μmol, 0.1 eq), TEA (698.72 mg, 6.91 mmol, 961.10 μL, 10 eq) in DCM (10 mL) was added dropwise 3-(dimethylamino)propanoyl chloride (594.02 mg, 3.45 mmol, 5 eq, HCl) in DCM (5 mL) at 0° C. The mixture was degassed and purged with N₂ for 3 times, and then stirred at 25° C. for 4 hours under N₂ atmosphere. The reaction mixture was quenched by addition of 40 mL H₂O, and extracted with 400 mL EtOAc (200 mL×2). The combined organic layers were washed with brine 100 mL, dried over Na₂SO₄, filtered, and concentrated under reduced pressure to give a residue. The residue was purified by prep-HPLC (column: Phenomenex Luna C18 100×30 mm×5 m; mobile phase: [water(HCl)-ACN]; B %: 40%-70%, 10 minutes) and prep-TLC (SiO₂, petroleum ether/ethyl acetate=1:10, 2% NH₃·H₂O) to give [8-(1-hexylheptoxy)-8-oxo-octyl](2S)-4-[3-(dimethylamino)propanoyloxy]-1-(6-oxo-6-undecoxy-hexyl)pyrrolidine-2-carboxylate (76 mg, 92.43 mol, 100% purity, 24.18% yield) as colorless oil.

¹H NMR (400 MHz, CDCl₃), 5.20-5.27 (m, 1H), 4.85-4.88 (m, 1H), 4.03-4.12 (m, 4H), 3.05-3.57 (m, 2H), 1.98-2.62 (m, 19H), 1.62-1.64 (m, 8H), 1.45-1.52 (m, 6H), 1.22-1.41 (m, 40H), 0.88 (t, J=6.8 Hz, 9H). LCMS: (M+H⁺): 823.6 @8.281 minutes.

### 8.24. Synthesis of Compound 2342

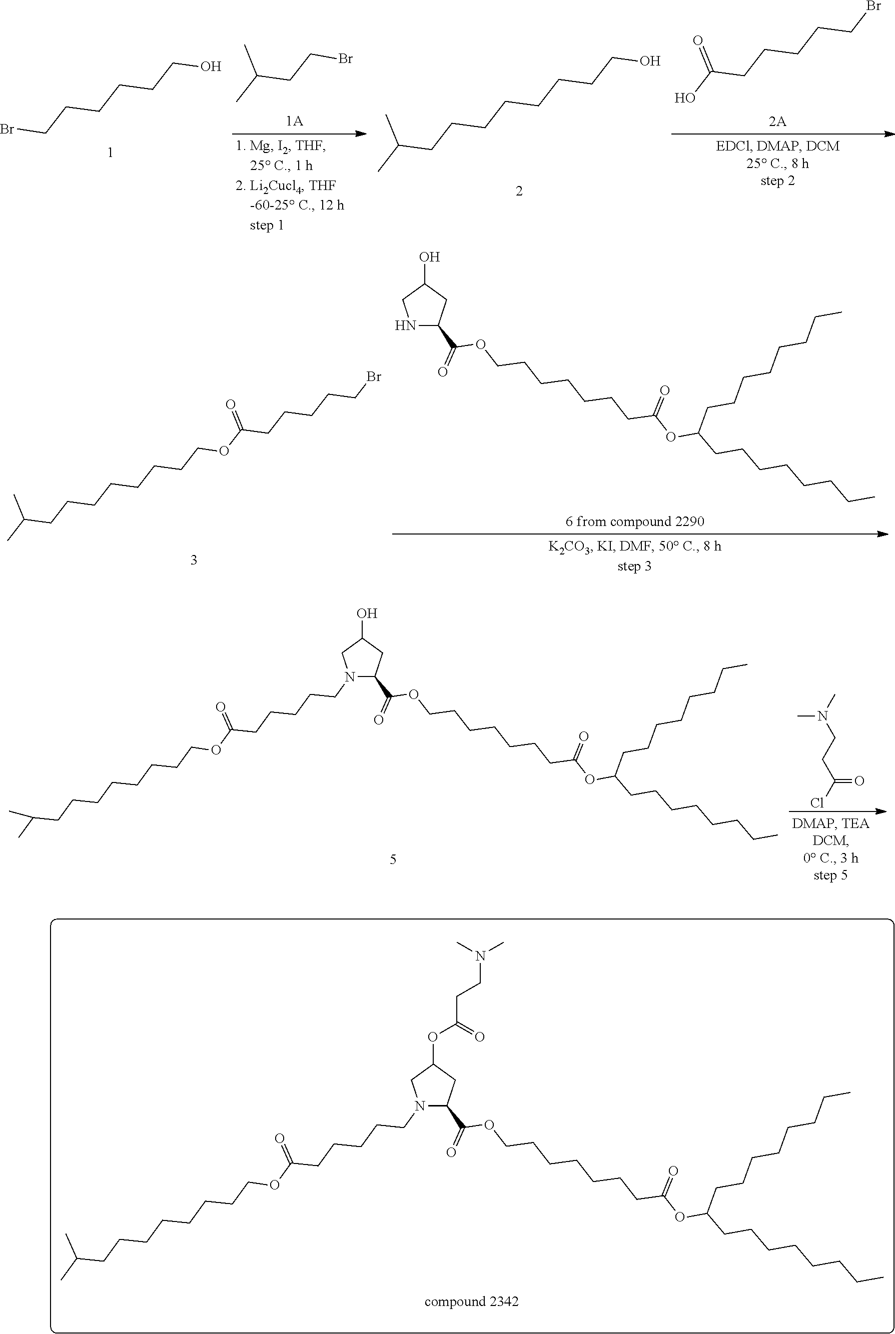

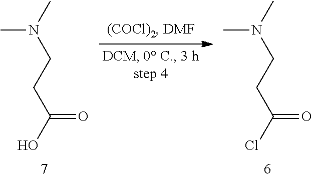

### Step 1

To a solution of Mg (3.84 g, 158.00 mmol, 1.19 eq) in THF (400 mL) was added I₂ (168.04 mg, 662.06 mol, 133.36 μL, 0.005 eq) and 1-bromo-3-methyl-butane (20 g, 132.42 mmol, 16.66 mL, 1 eq). The mixture was stirred at 25° C. for 1 hour under N₂ atmosphere. The mixture was added into a solution of 6-bromohexan-1-ol (5 g, 27.60 mmol, 1.45 mL, 1 eq) in THF (50 mL) and then dilithium tetrachlorocopper(II) (0.1 M, 13.8 mL, 0.05 eq) was added at −60° C. The mixture was stirred at 25° C. for 12 hours. The reaction mixture was quenched by addition of 100 mL H₂O at 0° C., and then extracted with 150 mL EtOAc (50 mL×3). The combined organic layers were washed with 150 mL saturated brine (50 mL×3), dried over Na₂SO₄, filtered, and concentrated under reduced pressure to give a residue. The residue was purified by column chromatography (SiO₂, petroleum ether/ethyl acetate=50/1 to 3/1) to give 9-methyldecan-1-ol (4 g, 23.21 mmol, 84.07% yield) as colorless oil.

¹H NMR (400 MHz, CDCl₃), 3.64 (t, J=6.8 Hz, 2H), 1.53-1.57 (m, 3H), 1.14-1.34 (m, 12H), 0.86 (d, J=6.8 Hz, 6H).

### Step 2

To a solution of 9-methyldecan-1-ol (4 g, 23.21 mmol, 1 eq) and 6-bromohexanoic acid (4.53 g, 23.21 mmol, 1 eq) in DCM (30 mL) was added EDCI (5.34 g, 27.86 mmol, 1.2 eq) and DMAP (1.42 g, 11.61 mmol, 0.5 eq). The mixture was stirred at 25° C. for 8 hours. The reaction mixture was quenched by addition of 100 mL H₂O at 0° C., and then extracted with 150 mL EtOAc (50 mL×3). The combined organic layers were washed with 150 mL brine (50 mL×3), dried over Na₂SO₄, filtered, and concentrated under reduced pressure to give a residue. The residue was purified by column chromatography (SiO₂, petroleum ether/ethyl acetate=1/0 to 50/1) to give 9-methyldecyl 6-bromohexanoate (4.5 g, 12.88 mmol, 55.49% yield) as yellow oil.

### Step 3

To a solution of 9-methyldecyl 6-bromohexanoate (1.50 g, 4.30 mmol, 1.1 eq) in DMF (20 mL) was added K₂CO₃ (1.62 g, 11.72 mmol, 3 eq), KI (324.37 mg, 1.95 mmol, 0.5 eq) and [8-(1-octylnonoxy)-8-oxo-octyl](2S)-4-hydroxypyrrolidine-2-carboxylate (2 g, 3.91 mmol, 1 eq). The mixture was stirred at 50° C. for 8 hours. The reaction mixture was quenched by addition of 50 mL H₂O at 0° C., and then extracted with 90 mL EtOAc (30 mL×3). The combined organic layers were washed with 90 mL brine (30 mL×3), dried over Na₂SO₄, filtered, and concentrated under reduced pressure to give a residue. The residue was purified by column chromatography (SiO₂, petroleum ether/ethyl acetate=10/1 to 0/1, added 0.1% NH₃·H₂O to give [8-(1-octylnonoxy)-8-oxo-octyl](2S)-4-hydroxy-1-[6-(9-methyldecoxy)-6-oxo-hexyl]pyrrolidine-2-carboxylate (2 g, 2.56 mmol, 65.59% yield) as colorless oil.

¹H NMR (400 MHz, CDCl₃), 4.86 (t, J=6.4 Hz, 1H), 4.17-4.40 (m, 1H), 4.03-4.09 (m, 4H), 3.23-3.65 (m, 3H), 2.31-3.21 (m, 3H), 2.27-2.98 (m, 4H), 1.27-1.70 (m, 60H), 1.24-1.59 (m, 3H), 0.85-0.87 (m, 12H).

### Step 4

To a solution of 3-(dimethylamino)propanoic acid (700 mg, 4.56 mmol, 1 eq, HCl) in DCM (10 mL) was added DMF (33.31 mg, 455.71 mol, 35.06 μL, 0.1 eq) and (COCl)₂ (694.10 mg, 5.47 mmol, 478.69 μL, 1.2 eq). The mixture was stirred at 0° C. for 3 hours. The mixture was concentrated under reduced pressure to give 3-(dimethylamino)propanoyl chloride (780 mg, crude, HCl) as a white solid.

### Step 5

To a solution of [8-(1-octylnonoxy)-8-oxo-octyl](2S)-4-hydroxy-1-[6-(9-methyldecoxy)-6-oxo-hexyl]pyrrolidine-2-carboxylate (700 mg, 897.20 μmol, 1 eq) in DCM (10 mL) was added TEA (907.86 mg, 8.97 mmol, 1.25 mL, 10 eq), DMAP (21.92 mg, 179.44 μmol, 0.2 eq) and 3-(dimethylamino)propanoyl chloride (771.82 mg, 4.49 mmol, 5 eq, HCl). The mixture was stirred at 0° C. for 3 hours. The reaction mixture was quenched by addition of 20 mL H₂O at 0° C., and then extracted with 30 mL EtOAc (10 mL×3). The combined organic layers were washed with 30 mL brine (10 mL×3), dried over Na₂SO₄, filtered, and concentrated under reduced pressure to give a residue. The residue was purified by column chromatography (SiO₂, petroleum ether/ethyl acetate=20/1 to 0/1, added 0.1% NH₃·H₂O) to give [8-(1-octylnonoxy)-8-oxo-octyl](2S)-4-[3-(dimethylamino) propanoyloxy]-1-[6-(9-methyldecoxy)-6-oxo-hexyl]pyrrolidine-2-carboxylate (500 mg, 568.61 μmol, 63.38% yield) as colorless oil.

1H NMR (400 MHz, CDCl₃), 5.20-5.27 (m, 1H), 4.86 (t, J=6.0 Hz, 1H), 4.03-4.13 (m, 4H), 3.08-3.53 (m, 2H), 2.49-2.79 (m, 7H), 2.24-2.31 (m, 11H), 2.03-2.17 (m, 1H), 1.59-1.64 (m, 8H), 1.48-1.53 (m, 7H), 1.26-1.34 (m, 42H), 1.14-1.16 (m, 2H), 0.85-0.89 (m, 12H).

LCMS: (M+H⁺): 879.7@10.027&10.102 minutes.

### 8.25. Synthesis of Compound 2343

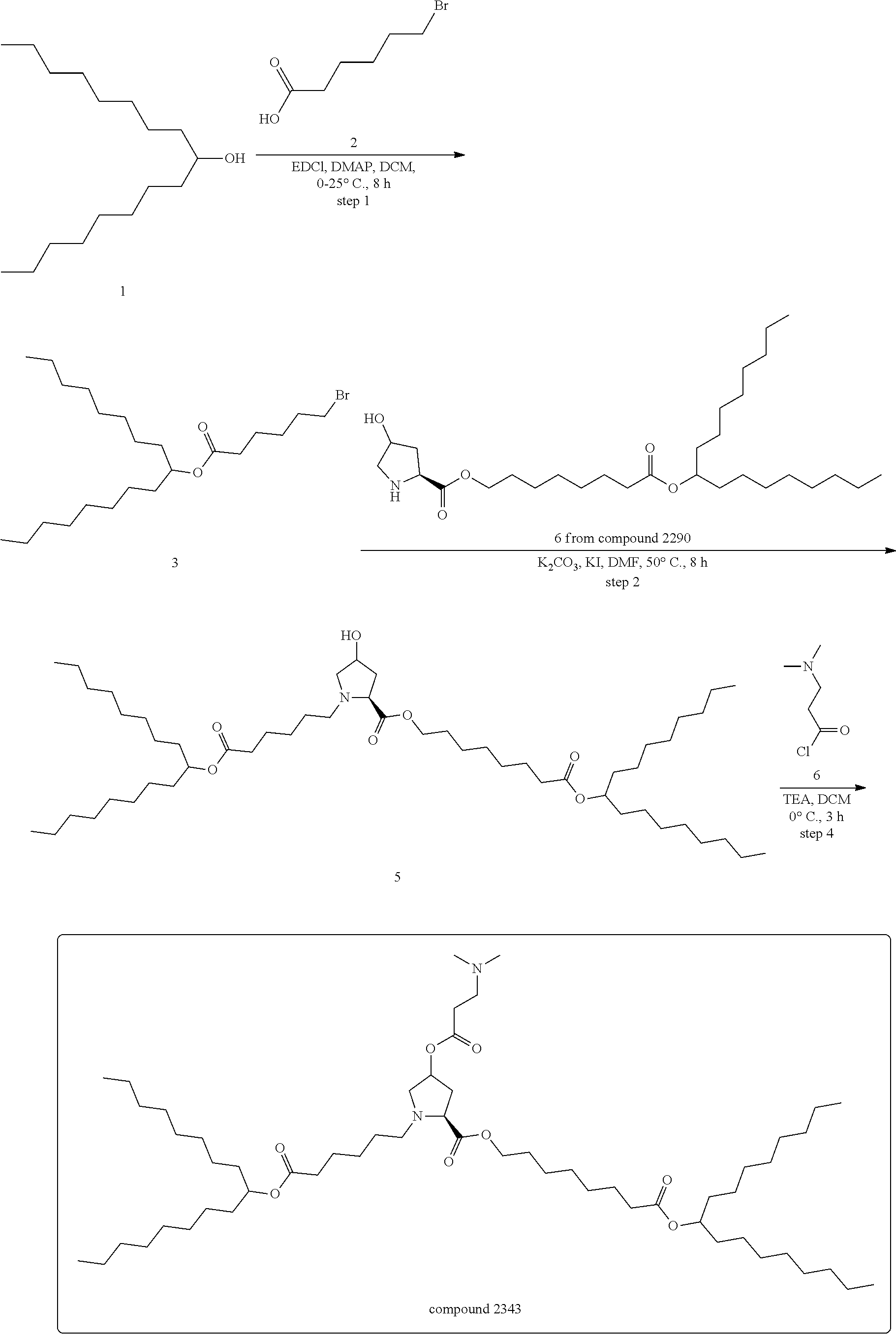

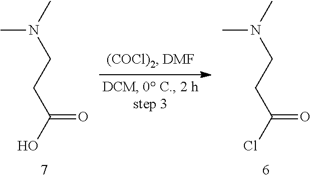

### Step 1

To a solution of heptadecan-9-ol (5 g, 19.50 mmol, 1 eq) and 6-bromohexanoic acid (3.80 g, 19.50 mmol, 1 eq) in DCM (100 mL) was added EDCI (4.48 g, 23.39 mmol, 1.2 eq) and DMAP (1.19 g, 9.75 mmol, 0.5 eq) at 0° C. The mixture was stirred at 25° C. for 8 hours. The reaction mixture was quenched by addition of 100 mL H₂O at 0° C., and then extracted with 300 mL EtOAc (100 mL×3). The combined organic layers were washed with 300 mL brine (100 mL×3), dried over Na₂SO₄, filtered, and concentrated under reduced pressure to give a residue. The residue was purified by column chromatography (SiO₂, petroleum ether/ethyl acetate=1/0 to 20/1) to give 1-octylnonyl 6-bromohexanoate (5.8 g, 13.38 mmol, 68.63% yield) as colorless oil.

¹H NMR (400 MHz, CDCl₃), 4.86-4.89 (m, 1H), 3.41 (t, J=6.8 Hz, 2H), 2.31 (t, J=7.2 Hz, 2H), 1.85-1.92 (m, 2H), 1.62-1.68 (m, 2H), 1.48-1.52 (m, 6H), 1.24-1.30 (m, 24H), 0.88 (t, J=6.4 Hz, 6H).

### Step 2

To a solution of 1-octylnonyl 6-bromohexanoate (1.86 g, 4.30 mmol, 1.1 eq) in DMF (20 mL) was added K₂CO₃ (1.62 g, 11.72 mmol, 3 eq), KI (324.37 mg, 1.95 mmol, 0.5 eq) and [8-(1-octylnonoxy)-8-oxo-octyl](2S)-4-hydroxypyrrolidine-2-carboxylate (2 g, 3.91 mmol, 1 eq). The mixture was stirred at 50° C. for 8 hours. The reaction mixture was quenched by addition of 30 mL H₂O at 0° C., and then extracted with 60 mL EtOAc (20 mL×3). The combined organic layers were washed with 60 mL brine (20 mL×3), dried over Na₂SO₄, filtered, and concentrated under reduced pressure to give a residue. The residue was purified by column chromatography (SiO₂, petroleum ether/ethyl acetate=10/1 to 0/1, added 0.1% NH₃—H₂O) to give [8-(1-octylnonoxy)-8-oxo-octyl](2S)-4-hydroxy-1-[6-(1-octylnonoxy)-6-oxo-hexyl]pyrrolidine-2-carboxylate (1.8 g, 2.08 mmol, 53.29% yield) as colorless oil.

¹H NMR (400 MHz, CDCl₃), 4.87 (t, J=6.0 Hz, 2H), 4.28-4.49 (m, 1H), 4.10-4.13 (m, 2H), 3.63-3.66 (m, 1H), 3.06-3.26 (m, 1H), 2.50-2.63 (m, 2H), 2.28 (t, J=7.2 Hz, 4H), 1.62-1.64 (m, 8H), 1.50-1.51 (m, 8H), 1.26-1.34 (m, 56H), 0.88 (t, J=6.8 Hz, 12H).

### Step 3

To a solution of 3-(dimethylamino)propanoic acid (500 mg, 3.26 mmol, 1 eq, HCl) in DCM (10 mL) was added DMF (237.92 mg, 3.26 mmol, 250.45 μL, 1 eq) and (COCl)₂ (495.78 mg, 3.91 mmol, 341.92 μL, 1.2 eq). The mixture was stirred at 0° C. for 2 hours. The mixture was concentrated under reduced pressure to give 3-(dimethylamino)propanoyl chloride (560 mg, crude, HCl) as a white solid.

### Step 4

To a solution of [8-(1-octylnonoxy)-8-oxo-octyl](2S)-4-hydroxy-1-[6-(1-octylnonoxy)-6-oxo-hexyl]pyrrolidine-2-carboxylate (500 mg, 578.46 mol, 1 eq) in DCM (10 mL) was added TEA (585.34 mg, 5.78 mmol, 805.14 μL, 10 eq) and 3-(dimethylamino)propanoyl chloride (497.63 mg, 2.89 mmol, 5 eq, HCl). The mixture was stirred at 0° C. for 3 hours. The reaction mixture was quenched by addition of 10 mL H₂O at 0° C., and then extracted with 30 mL EtOAc (10 mL×3). The combined organic layers were washed with brine (10 mL×3), dried over Na₂SO₄, filtered, and concentrated under reduced pressure to give a residue. The residue was purified by prep-HPLC (column: Phenomenex Luna C18 100×30 mm×5 m; mobile phase: [water(HCl)-ACN]; B %: 45%-75%, 10 minutes). Then the mixture was adjusted to pH=o 8 with saturated NaHCO₃, and extracted with 45 mL EtOAc (15 mL×3). The combined organic layers were dried over Na₂SO₄, filtered, and concentrated under reduced pressure to give [8-(1-octylnonoxy)-8-oxo-octyl](2S)-4-[3-(dimethylamino) propanoyloxy]-1-[6-(1-octylnonoxy)-6-oxo-hexyl]pyrrolidine-2-carboxylate (70 mg, 72.65 μmol, 12.56% yield) as yellow oil.

¹H NMR (400 MHz, CDCl₃), 5.20-5.26 (m, 1H), 4.84-4.88 (m, 2H), 4.09-4.14 (m, 2H), 3.11-3.52 (m, 2H), 2.49-2.63 (m, 7H), 2.28-2.30 (m, 10H), 2.25-2.26 (m, 2H), 1.63-1.65 (m, 10H), 1.51-1.61 (m, 4H), 1.26-1.34 (m, 58H), 0.88 (t, J=6.4 Hz, 12H).

LCMS: (M+H⁺): 963.8@13.048&13.134 minutes

### 8.26. Synthesis of Compound 2344

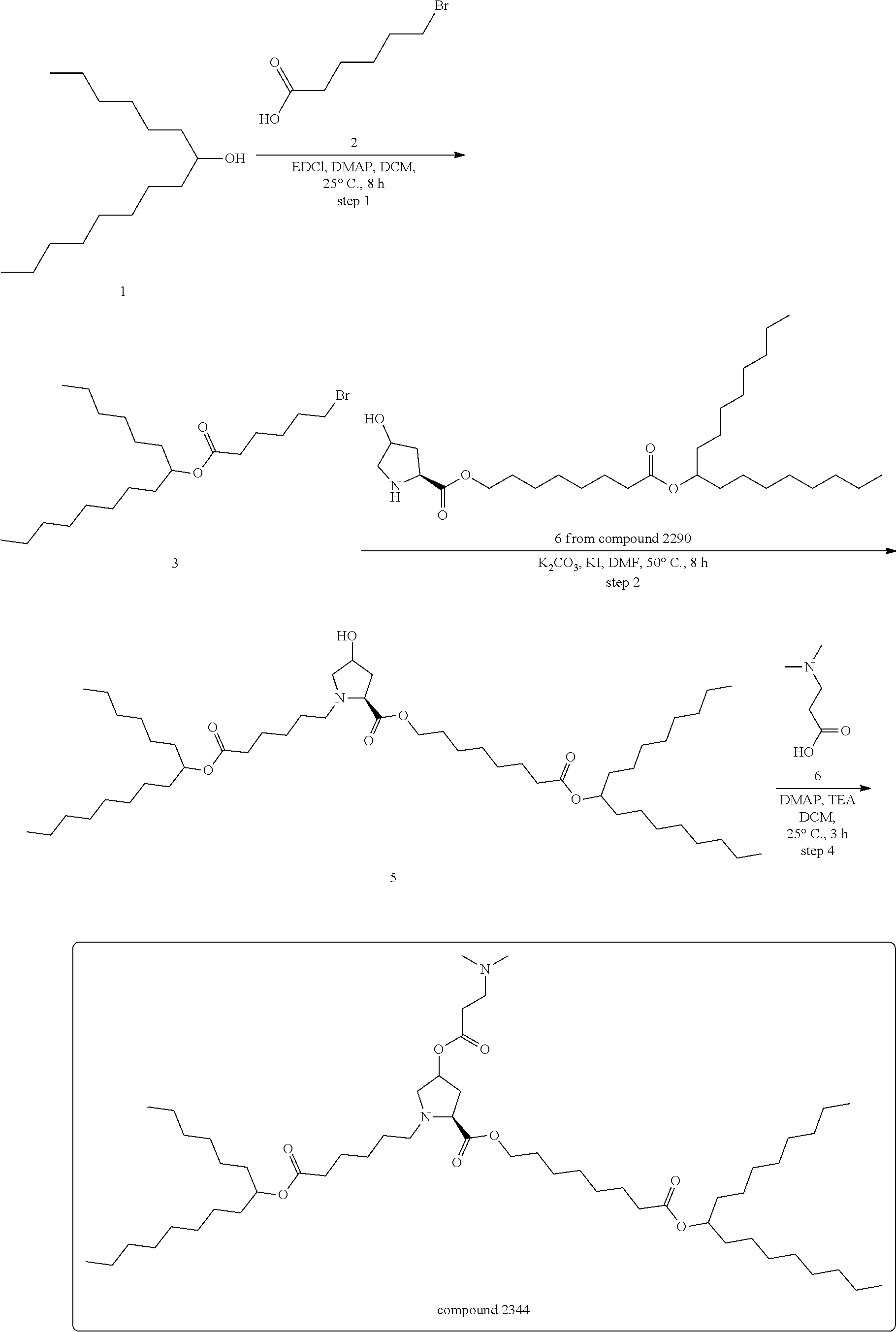

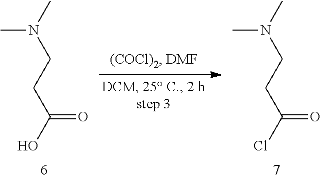

### Step 1

To a solution of pentadecan-7-ol (2.5 g, 10.95 mmol, 1 eq) and 6-bromohexanoic acid (2.13 g, 10.95 mmol, 1 eq) in DCM (100 mL) was added EDCI (2.52 g, 13.13 mmol, 1.2 eq) and DMAP (668.57 mg, 5.47 mmol, 0.5 eq). The mixture was stirred at 25° C. for 8 hours. The reaction mixture was quenched by addition of 100 mL H₂O at 0° C., and then extracted with 150 mL EtOAc (50 mL×3). The combined organic layers were washed with 150 mL brine (50 mL×3), dried over Na₂SO₄, filtered, and concentrated under reduced pressure to give a residue. The residue was purified by column chromatography (SiO₂, petroleum ether/ethyl acetate=1/0 to 10/1) to give 1-hexylnonyl 6-bromohexanoate (5 g, 12.33 mmol, 56.34% yield) as colorless oil.

### Step 2

To a solution of 1-hexylnonyl 6-bromohexanoate (950.69 mg, 2.34 mmol, 1.2 eq) and [8-(1-octylnonoxy)-8-oxo-octyl](2S)-4-hydroxypyrrolidine-2-carboxylate (1 g, 1.95 mmol, 1 eq) in DMF (50 mL) was added K₂CO₃ (810.18 mg, 5.86 mmol, 3 eq) and KI (162.18 mg, 976.99 μmol, 0.5 eq). The mixture was stirred at 50° C. for 8 hours. The reaction mixture was quenched by addition of 50 mL H₂O at 15° C., and then extracted with EtOAc (50 mL×3). The combined organic layers were washed with brine (50 mL×2), dried over Na₂SO₄, filtered, and concentrated under reduced pressure to give a residue. The residue was purified by column chromatography (SiO₂, petroleum ether/ethyl acetate=1/0 to 8/1) to give [8-(1-octylnonoxy)-8-oxo-octyl](2S)-1-[6-(1-hexylnonoxy)-6-oxo-hexyl]-4-hydroxy-pyrrolidine-2-carboxylate (1.2 g, 1.43 mmol, 36.72% yield) as a yellow oil.

1H NMR (400 MHz, CDCl₃), 4.80-4.94 (m, 2H), 4.09-4.15 (m, 2H), 2.99-3.99 (m, 4H), 2.44-2.60 (m, 2H), 2.28 (t, J=7.6 Hz, 4H), 1.61-1.70 (m, 8H), 1.49-1.52 (m, 6H), 1.16-1.38 (m, 56H), 0.88 (t, J=7.2 Hz, 12H).

### Step 3

To a solution of 3-(dimethylamino)propanoic acid (300 mg, 1.95 mmol, 1 eq, HCl) in DCM (5 mL) was added (COCl)₂ (1.24 g, 9.77 mmol, 854.82 μL, 5 eq) and DMF (14.27 mg, 195.30 mol, 15.03 μL, 0.1 eq). The mixture was stirred at 25° C. for 2 hours. The reaction mixture was concentrated under reduced pressure to give 3-(dimethylamino)propanoyl chloride (336 mg, 1.95 mmol, 99.99% yield, HCl) as a yellow solid.

### Step 4

To the suspension of [8-(1-octylnonoxy)-8-oxo-octyl](2S)-1-[6-(1-hexylnonoxy)-6-oxo-hexyl]-4-hydroxy-pyrrolidine-2-carboxylate (500 mg, 597.86 mol, 1 eq), DMAP (36.52 mg, 298.93 mol, 0.5 eq) and TEA (302.49 mg, 2.99 mmol, 416.08 μL, 5 eq) in DCM (5 mL) was added dropwise 3-(dimethylamino)propanoyl chloride (308.59 mg, 1.79 mmol, 3 eq, HCl) in DCM (3 mL). The mixture was stirred at 25° C. for 3 hours under N₂ atmosphere. The reaction mixture was quenched by addition of 10 mL aqueous NaHCO₃ at 25° C., and then extracted with 30 mL EtOAc (10 mL×3). The combined organic layers were washed with 20 mL brine (10 mL×2), dried over Na₂SO₄, filtered, and concentrated under reduced pressure to give a residue. The residue was purified by column chromatography (SiO₂, petroleum ether/ethyl acetate=1/0 to 5/1) and purified by prep-HPLC (column: Phenomenex Luna C18 100×30 mm×5 m; mobile phase: [water(HCl)-ACN]; B %: 45%-75%, 10 minutes). Then the mixture was adjusted to pH=8 with saturated NaHCO₃, and extracted with 45 mL EtOAc (15 mL×3). The combined organic layers were dried over Na₂SO₄, filtered, and concentrated under reduced pressure to give [8-(1-octylnonoxy)-8-oxo-octyl](2S)-4-[3-(dimethylamino)propanoyloxy]-1-[6-(1-hexylnonoxy)-6-oxo-hexyl]pyrrolidine-2-carboxylate (220 mg, 231.65 mol, 47.11% yield) as colorless oil.

¹H NMR (400 MHz, CDCl₃), 5.20-5.30 (m, 1H), 4.84-4.90 (m, 2H), 4.09-4.15 (m, 2H), 3.43-3.55 (m, 1H), 3.08-3.26 (m, 1H), 2.20-2.80 (m, 18H), 1.98-2.20 (m, 1H), 1.60-1.70 (m, 6H), 1.45-1.55 (m, 10H), 1.20-1.38 (m, 52H), 0.88 (t, J=7.2 Hz, 12H).

LCMS: (M/2+H⁺): 935.7 @11.390/11.490 minutes.

### 8.27. Synthesis of Compound 2345

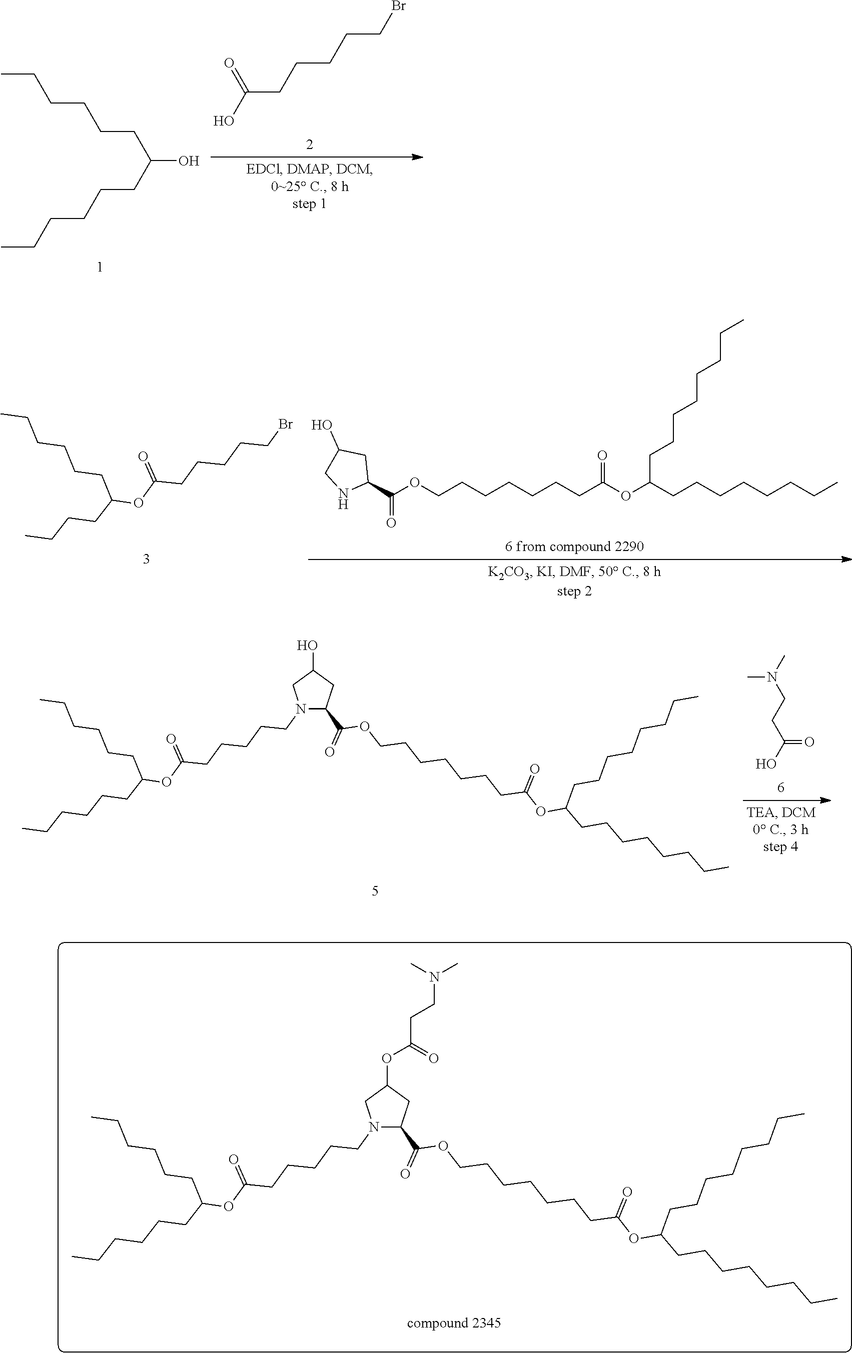

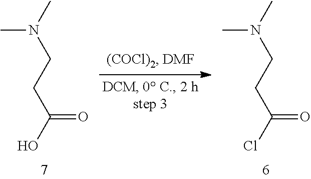

### Step 1

To a solution of tridecan-7-ol (5.14 g, 25.63 mmol, 1 eq) and 6-bromohexanoic acid (5 g, 25.63 mmol, 1 eq) in DCM (100 mL) was added EDCI (5.90 g, 30.76 mmol, 1.2 eq) and DMAP (1.57 g, 12.82 mmol, 0.5 eq) at 0° C. The mixture was stirred at 25° C. for 8 hours. The reaction mixture was quenched by addition of 100 mL H₂O at 0° C., and then extracted with 300 mL EtOAc (100 mL×3). The combined organic layers were washed with 300 mL brine (100 mL×3), dried over Na₂SO₄, filtered, and concentrated under reduced pressure to give a residue. The residue was purified by column chromatography (SiO₂, petroleum ether/ethyl acetate=1/0 to 20/1) to give 1-hexylheptyl 6-bromohexanoate (8 g, 21.20 mmol, 82.69% yield) as colorless oil.

¹H NMR (400 MHz, CDCl₃), 4.84-4.90 (m, 1H), 3.41 (t, J=6.8 Hz, 2H), 2.31 (t, J=7.2 Hz, 2H), 1.88-1.92 (m, 2H), 1.62-1.70 (m, 2H), 1.48-1.52 (m, 6H), 1.24-1.30 (m, 16H), 0.88 (t, J=6.4 Hz, 6H).

### Step 2

To a solution of [8-(1-octylnonoxy)-8-oxo-octyl](2S)-4-hydroxypyrrolidine-2-carboxylate (2 g, 3.91 mmol, 1 eq) in DMF (20 mL) was added K₂CO₃ (1.62 g, 11.72 mmol, 3 eq), KI (324.37 mg, 1.95 mmol, 0.5 eq) and 1-hexylheptyl 6-bromohexanoate (1.62 g, 4.30 mmol, 1.1 eq). The mixture was stirred at 50° C. for 8 hours. The reaction mixture was quenched by addition of 50 mL H₂O at 0° C., and then extracted with 90 mL EtOAc (30 mL×3). The combined organic layers were washed with 90 mL brine (30 mL×3), dried over Na₂SO₄, filtered, and concentrated under reduced pressure to give a residue. The residue was purified by column chromatography (SiO₂, petroleum ether/ethyl acetate=10/1 to 1/1, added 0.1% NH₃·H₂O) to give [8-(1-octylnonoxy)-8-oxo-octyl](2S)-1-[6-(1-hexylheptoxy)-6-oxo-hexyl]-4-hydroxy-pyrrolidine-2-carboxylate (1.7 g, 2.10 mmol, 53.82% yield) as colorless oil.

¹H NMR (400 MHz, CDCl₃), 4.86 (t, J=6.0 Hz, 2H), 4.25-4.48 (m, 1H), 4.10-4.13 (m, 2H), 3.05-3.65 (m, 3H), 2.49-2.63 (m, 7H), 1.50-1.64 (m, 18H), 1.26-1.34 (m, 48H), 0.88 (t, J=6.4 Hz, 12H).

### Step 3

To a solution of 3-(dimethylamino)propanoic acid (500 mg, 3.26 mmol, 1 eq, HCl) in DCM (10 mL) was added DMF (23.79 mg, 325.51 mol, 25.04 μL, 0.1 eq) and (COCl)₂ (495.78 mg, 3.91 mmol, 341.92 μL, 1.2 eq). The mixture was stirred at 0° C. for 2 hours. The mixture was concentrated under reduced pressure to give 3-(dimethylamino)propanoyl chloride (560 mg, crude, HCl) as a white solid.

### Step 4

To a solution of [8-(1-octylnonoxy)-8-oxo-octyl](2S)-1-[6-(1-hexylheptoxy)-6-oxo-hexyl]-4-hydroxy-pyrrolidine-2-carboxylate (500 mg, 618.61 mol, 1 eq) in DCM (10 mL) was added TEA (625.97 mg, 6.19 mmol, 861.03 μL, 10 eq) and 3-(dimethylamino)propanoyl chloride (532.17 mg, 3.09 mmol, 5 eq, HCl). The mixture was stirred at 0° C. for 3 hours. The reaction mixture was quenched by addition of 10 mL H₂O at 0° C., and then extracted with 30 mL EtOAc (10 mL×3). The combined organic layers were washed with 30 mL brine (10 mL×3), dried over Na₂SO₄, filtered, and concentrated under reduced pressure to give a residue. The residue was purified by prep-HPLC (column: Phenomenex Luna C18 100×30 mm×5 μm; mobile phase: [water(HCl)-ACN]; B %: 40%-70%, 10 minutes). Then the solution was adjusted to pH=8 with saturated NaHCO₃, and extracted with 30 mL EtOAc (10 mL×3). The combined organic layers were dried over Na₂SO₄, filtered, and concentrated under reduced pressure to give [8-(1-octylnonoxy)-8-oxo-octyl](2S)-4-[3-(dimethylamino) propanoyloxy]-1-[6-(1-hexylheptoxy)-6-oxo-hexyl]pyrrolidine-2-carboxylate (48 mg, 52.90 μmol, 8.55% yield) as colorless oil.

¹H NMR (400 MHz, CDCl₃), 5.19-5.21 (m, 1H), 4.86 (t, J=6.0 Hz, 2H), 4.07-4.14 (m, 2H), 3.11-3.45 (m, 2H), 2.48-2.64 (m, 7H), 1.86-2.30 (m, 12H), 1.63 (s, 5H), 1.50 (d, J=5.2 Hz, 10H), 1.26-1.34 (m, 50H), 0.88 (t, J=6.4 Hz, 12H). LCMS: (M+H⁺): 907.7@11.723&11.808 minutes.

### 8.28. Synthesis of Compound 2348

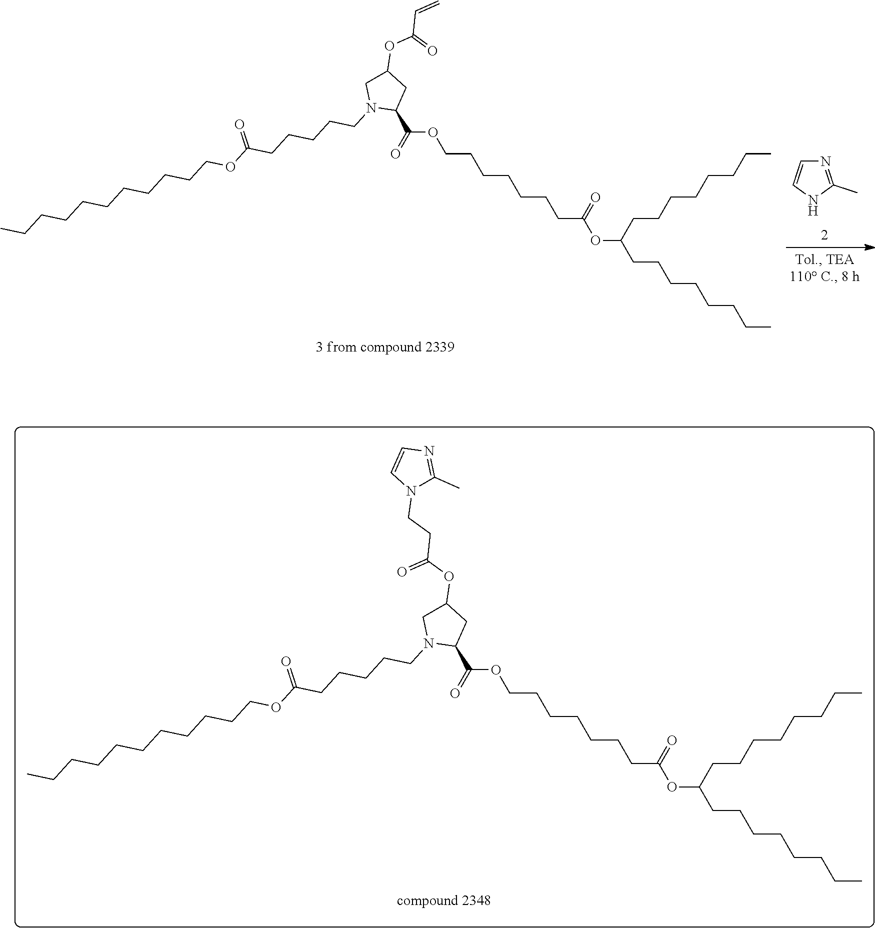

A mixture of [8-(1-octylnonoxy)-8-oxo-octyl](2S)-1-(6-oxo-6-undecoxy-hexyl)-4-prop-2-enoyloxy-pyrrolidine-2-carboxylate (200 mg, 239.73 mol, 1 eq), 2-methyl-1H-imidazole (59.05 mg, 719.20 mol, 3 eq) and TEA (24.26 mg, 239.73 mol, 33.37 μL, 1 eq) in toluene (10 mL) was degassed and purged with N₂ for 3 times, and then the mixture was stirred at 110° C. for 8 hours under N₂ atmosphere. The reaction mixture was diluted with 20 mL H₂O, and extracted with 100 mL EtOAc (50 mL×2). The combined organic layers were washed with 20 mL brine, dried over Na₂SO₄, filtered, and concentrated under reduced pressure to give a residue. The residue was purified by column chromatography (SiO₂, petroleum ether/ethyl acetate=5/1 to 1/1, 3% NH₃·H₂O), column chromatography (SiO₂, petroleum ether/ethyl acetate=3/1 to 0/1, 3% NH₃·H₂O), and prep-TLC (SiO₂, petroleum ether/ethyl acetate=1:8, 1% NH₃—H₂O) to give [8-(1-octylnonoxy)-8-oxo-octyl](2S)-4-[3-(2-methylimidazol-1-yl)propanoyloxy]-1-(6-oxo-6-undecoxy-hexyl) pyrrolidine-2-carboxylate (89 mg, 97.12 mol, 40.51% yield, 100% purity) as colorless oil.

¹H NMR (400 MHz, CDCl₃), 6.85-6.92 (m, 2H), 5.25-5.27 (m, 1H), 4.85-4.90 (m, 1H), 4.03-4.16 (m, 6H), 3.05-3.50 (m, 2H), 2.38-2.60 (m, 8H), 2.25-2.35 (m, 5H), 1.97-2.10 (m, 1H), 1.59-1.64 (m, 8H), 1.42-1.50 (m, 6H), 1.21-1.35 (m, 48H), 0.88 (t, J=6.8 Hz, 9H).

LCMS: (M+H⁺): 916.6 @9.723 minutes.

### 8.29. Synthesis of Compound 2349

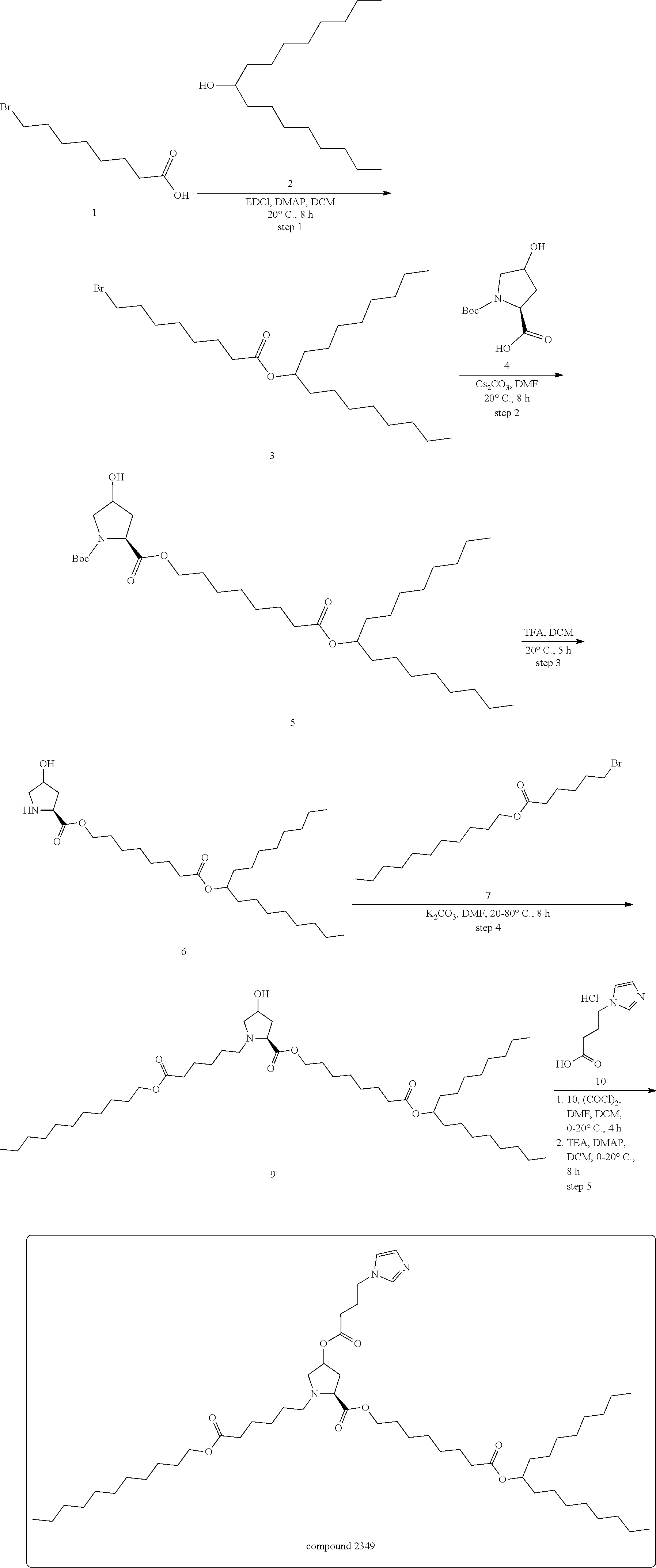

### Step 1

To a solution of 8-bromooctanoic acid (5 g, 22.41 mmol, 1.2 eq) in DCM (50 mL) was added EDCI (5.37 g, 28.01 mmol, 1.5 eq), DMAP (456.31 mg, 3.74 mmol, 0.2 eq) and heptadecan-9-ol (4.79 g, 18.68 mmol, 1 eq) at 20° C. The mixture was degassed and purged with N₂ for 3 times, and then stirred at 20° C. for 8 hours under N₂ atmosphere. The reaction mixture was concentrated under reduced pressure to give a residue. The residue diluted with 500 mL H₂O, and then extracted with 800 mL EtOAc (400 mL×2). The combined organic layers were washed with 500 mL brine, dried over Na₂SO₄, filtered, and concentrated under reduced pressure to give a residue. The residue was purified by column chromatography (SiO₂, petroleum ether/ethyl acetate=1/0 to 10/1) to give 1-octylnonyl 8-bromooctanoate (24 g, 52.00 mmol, 92.81% yield) as colourless oil.

### Step 2

To a solution of 1-octylnonyl 8-bromooctanoate (5 g, 10.83 mmol, 1.2 eq) and (2S)-1-tert-butoxycarbonyl-4-hydroxy-pyrrolidine-2-carboxylic acid (2.09 g, 9.03 mmol, 1 eq) in DMF (70 mL) was added Cs₂CO₃ (6.47 g, 19.86 mmol, 2.2 eq) at 20° C. The mixture was degassed and purged with N₂ for 3 times, and then stirred at 20° C. for 8 hours under N₂ atmosphere. The reaction mixture was filtered and diluted with 50 mL H₂O, and then extracted with 200 mL EtOAc (100 mL×2). The combined organic layers were washed with 300 mL brine (150 mL×2), dried over Na₂SO₄, filtered, and concentrated under reduced pressure to give a residue. The residue was purified by column chromatography (SiO₂, petroleum ether/ethyl acetate=8/1 to 3/1) to give O1-tert-butyl O2-[8-(1-octylnonoxy)-8-oxo-octyl](2S)-4-hydroxypyrrolidine-1,2-dicarboxylate (27 g, 44.13 mmol, 97.76% yield) as colourless oil.

¹H NMR (400 MHz, CDCl₃), 4.85-4.89 (m, 1H), 4.11-4.55 (m, 4H), 3.35-3.75 (m, 2H), 2.05-2.35 (m, 4H), 1.55-1.63 (m, 10H), 1.26-1.50 (m, 37H), 0.88 (t, J=6.8 Hz, 6H).

### Step 3

To a solution of O1-tert-butyl O2-[8-(1-octylnonoxy)-8-oxo-octyl](2S)-4-hydroxypyrrolidine-1,2-dicarboxylate (10 g, 16.34 mmol, 1 eq) in DCM (60 mL) was added TFA (46.05 g, 403.87 mmol, 30 mL, 24.71 eq) at 20° C. The mixture was stirred at 20° C. for 5 hours. The reaction mixture was concentrated under reduced pressure to get a residue. The reaction mixture was adjusted to pH=7.0 with aqueous saturated NaHCO₃ and extracted with 100 mL EtOAc (25 mL×4). The combined organic layers were dried over Na₂SO₄, filtered, and concentrated under reduced pressure to give [8-(1-octylnonoxy)-8-oxo-octyl](2S)-4-hydroxypyrrolidine-2-carboxylate (7.14 g, 13.95 mmol, 85.37% yield) as yellow oil.

### Step 4

To a solution of [8-(1-octylnonoxy)-8-oxo-octyl](2S)-4-hydroxypyrrolidine-2-carboxylate (7.14 g, 13.95 mmol, 1 eq) and undecyl 6-bromohexanoate (5.85 g, 16.74 mmol, 1.2 eq) in DMF (100 mL) was added K₂CO₃ (5.78 g, 41.85 mmol, 3 eq) at 20° C. The mixture was stirred at 80° C. for 8 hours. The reaction mixture was filtered and concentrated under reduced pressure to give a residue. The reaction mixture was diluted with 300 mL H₂O and extracted with 600 mL EtOAc (200 mL×3). The combined organic layers were washed with 150 mL brine (50 ml×3), dried over Na₂SO₄, filtered, and concentrated under reduced pressure to give a residue. The residue was purified by column chromatography (SiO₂, petroleum ether/ethyl acetate=5/1 to 0/1) to give [8-(1-octylnonoxy)-8-oxo-octyl](2S)-4-hydroxy-1-(6-oxo-6-undecoxy-hexyl)pyrrolidine-2-carboxylate (9.6 g, crude) as yellow oil.

¹H NMR (400 MHz, CDCl₃), 4.76-4.82 (m, 1H), 4.22-4.52 (m, 1H), 4.10-4.20 (m, 2H), 4.07 (t, J=6.8 Hz, 2H), 3.40-3.68 (m, 1H), 3.02-3.24 (m, 1H), 2.45-2.78 (m, 3H), 2.25-2.33 (m, 4H), 1.86-2.17 (m, 2H), 1.51-1.56 (m, 8H), 1.42-1.44 (m, 6H), 1.19-1.38 (m, 48H), 0.80 (t, J=6.4 Hz, 9H).

### Step 5

To a solution of 4-imidazol-1-ylbutanoic acid (0.35 g, 2.27 mmol, 1 eq) in DCM (10 mL) was added oxalyl dichloride (1.44 g, 11.35 mmol, 993.65 μL, 5 eq) and DMF (19.00 mg, 259.94 μmol, 0.02 mL, 1.14e-1 eq) at 0° C. The mixture was degassed and purged with N₂ for 3 times, and then stirred at 20° C. for 4 hours under N₂ atmosphere. The reaction mixture was concentrated under reduced pressure to give 4-imidazol-1-ylbutanoyl chloride (0.5 g, crude, HCl) as colourless oil. The crude oil residue was dissolved with DCM (10 mL), and then added into a solution of [8-(1-octylnonoxy)-8-oxo-octyl](2S)-4-hydroxy-1-(6-oxo-6-undecoxy-hexyl)pyrrolidine-2-carboxylate (0.312 g, 399.89 mol, 1 eq), TEA (404.65 mg, 4.00 mmol, 556.60 μL, 10 eq) and DMAP (9.77 mg, 79.98 mol, 0.2 eq) in DCM (3 mL) at 0° C. The mixture was then stirred at 20° C. for 8 hours under N₂ atmosphere. The reaction mixture was concentrated under reduced pressure to get a residue. The reaction mixture was diluted with 20 mL aqueous saturated NaHCO₃, and extracted with 100 mL EtOAc (25 mL×4). The combined organic layers were dried over Na₂SO₄, filtered, and concentrated under reduced pressure to give a residue. The residue was purified by column chromatography (SiO₂, petroleum ether/ethyl acetate=8/1 to 0/1, 5% NH₃·H₂O) to give a residue. The residue was extracted with hexane (8 mL) and brine (8 mL). The hexane layer was dried over Na₂SO₄ and concentrated under reduced pressure to give [8-(1-octylnonoxy)-8-oxo-octyl](2S)-4-(4-imidazol-1-ylbutanoyloxy)-1-(6-oxo-6-undecoxy-hexyl)pyrrolidine-2-carboxylate (0.121 g, 132.04 mol, 34.57% yield) as yellow oil.

¹H NMR (400 MHz, CDCl₃), 7.49-7.50 (m, 1H), 7.07-7.08 (m, 1H), 6.92 (s, 1H), 5.25-5.27 (m, 1H), 4.85-4.88 (m, 1H), 4.03-4.13 (m, 6H), 3.24-3.52 (m, 2H), 2.28-2.59 (m, 10H), 1.98-2.10 (m, 3H), 1.60-1.66 (m, 8H), 1.40-1.50 (m, 6H), 1.26-1.34 (m, 48H), 0.88 (t, J=6.4 Hz, 9H).

LCMS: (M+H⁺): 916.8 @9.878 minutes.

### 8.30. Synthesis of Compound 2352

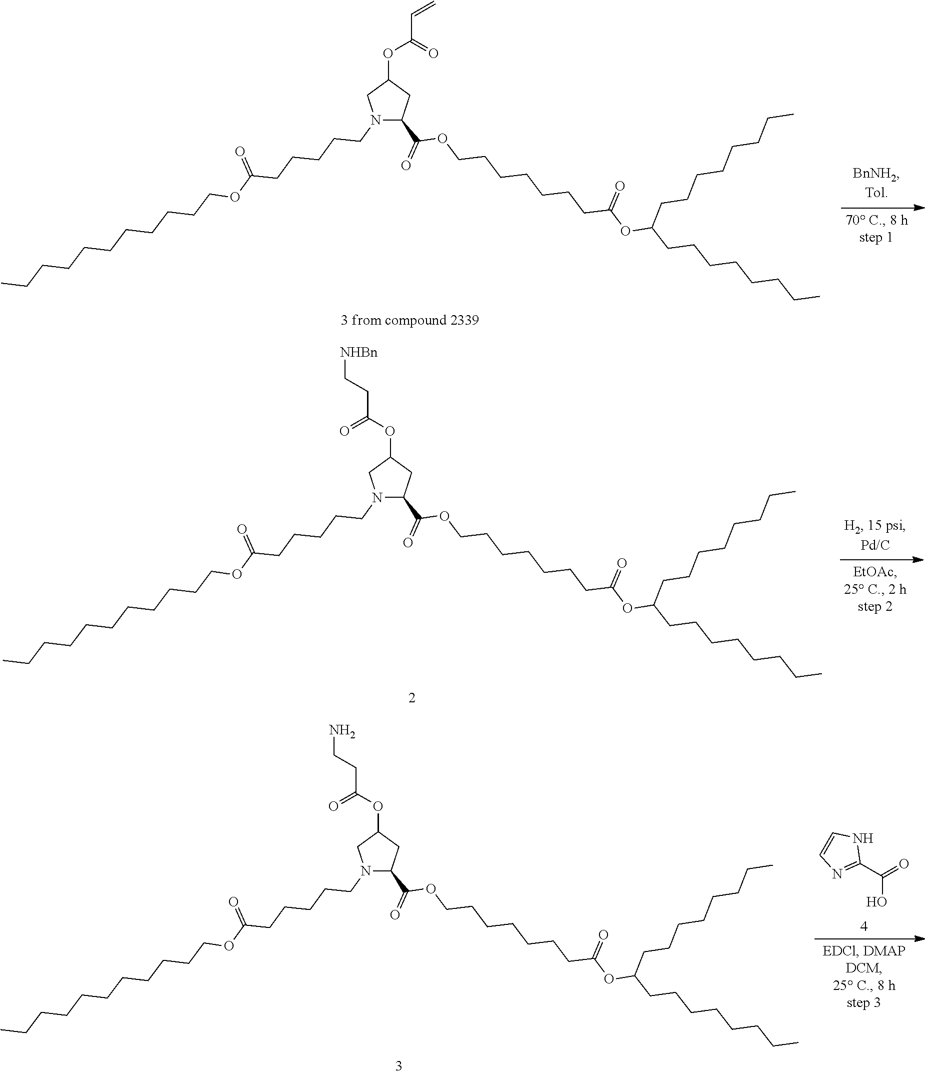

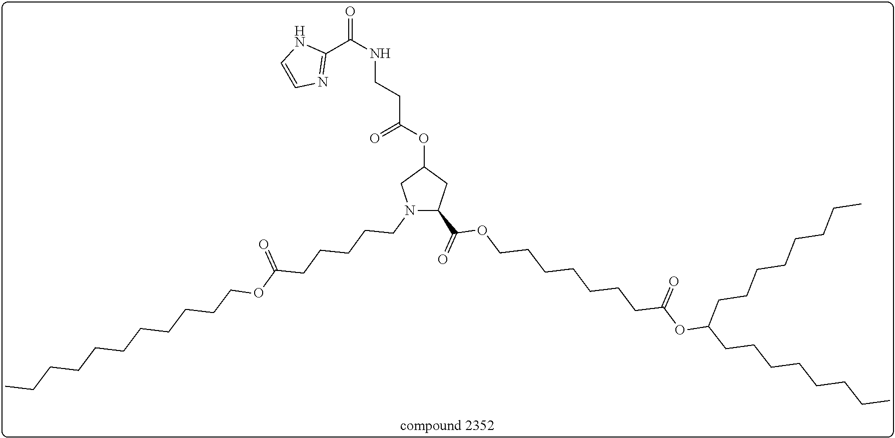 [SMILES: CCCCCCCCCCCOC(=O)CCCCCN1CC(OC(=O)CCNC(=O)c2ncc[nH]2)C[C@H]1C(=O)OCCCCCCCC(=O)OC(CCCCCCCC)CCCCCCCC]

### Step 1

A mixture of [8-(1-octylnonoxy)-8-oxo-octyl](2S)-1-(6-oxo-6-undecoxy-hexyl)-4-prop-2-enoyloxy-pyrrolidine-2-carboxylate (0.4 g, 479.47 mol, 1 eq) in Tol. (10 mL) was added phenylmethanamine (513.76 mg, 4.79 mmol, 522.65 μL, 10 eq) and then was degassed and purged with N₂ for 3 times. The mixture was stirred at 70° C. for 8 hours under N₂ atmosphere. The reaction mixture was diluted with 20 mL H₂O and extracted with 60 mL EtOAc (20 mL×3). The combined organic layers were dried over Na₂SO₄, filtered, and concentrated under reduced pressure to give a residue. The residue was purified by column chromatography (SiO₂, petroleum ether/ethyl acetate=20/1 to 5/1, 3% NH₃·H₂O) to give [8-(1-octylnonoxy)-8-oxo-octyl](2S)-4-[3-(benzylamino)propanoyloxy]-1-(6-oxo-6-undecoxy-hexyl)pyrrolidine-2-carboxylate (0.3 g, 315.48 mol, 65.80% yield, 99% purity) as yellow oil.

### Step 2

A solution of [8-(1-octylnonoxy)-8-oxo-octyl](2S)-4-[3-(benzylamino)propanoyloxy]-1-(6-oxo-6-undecoxy-hexyl)pyrrolidine-2-carboxylate (0.3 g, 318.67 mol, 1 eq) in EtOAc (10 mL) was added to a solution of Pd/C (0.3 g, 10% purity, 1 eq) in EtOAc (10 mL) under N₂. The suspension was degassed under vacuum and purged with H₂ several times. The mixture was stirred under H₂ (15 psi) at 25° C. for 2 hours. The mixture is filtered and the solvent was removed under reduced pressure to give [8-(1-octylnonoxy)-8-oxo-octyl](2S)-4-(3-aminopropanoyloxy)-1-(6-oxo-6-undecoxy-hexyl)pyrrolidine-2-carboxylate (0.2 g, 230.24 mol, 72.25% yield) as a white solid.

### Step 3

To a solution of 1H-imidazole-2-carboxylic acid (19.75 mg, 176.20 mol, 1.5 eq), EDCI (45.04 mg, 234.94 mol, 2 eq), DMAP (14.35 mg, 117.47 mol, 1 eq) in DCM (5 mL) was added [8-(1-octylnonoxy)-8-oxo-octyl](2S)-4-(3-aminopropanoyloxy)-1-(6-oxo-6-undecoxy-hexyl) pyrrolidine-2-carboxylate (0.1 g, 117.47 mol, 1 eq) in DCM (1 mL). The mixture was stirred at 25° C. for 8 hours. The reaction mixture was diluted with 20 mL H₂O and extracted with 60 mL EtOAc (20 mL×3). The combined organic layers were dried over Na₂SO₄, filtered, and concentrated under reduced pressure to give a residue. The residue was purified by column chromatography (SiO₂, petroleum ether/ethyl acetate=20/1 to 1/1, 3% NH₃·H₂O) and prep-TLC (SiO₂, PE:EtOAc=1:1, 1% NH₃·H₂O) to give [8-(1-octylnonoxy)-8-oxo-octyl](2S)-4-[3-(1H-imidazole-2-carbonylamino)propanoyloxy]-1-(6-oxo-6-undecoxy-hexyl)pyrrolidine-2-carboxylate (0.021 g, 26.45 mol, 22.510% yield, 100% purity) as colorless oil.

1H NMR (400 MHz, CDCl₃), 7.67 (brs, 1H), 7.15 (s, 2H), 5.21-5.39 (m, 1H), 4.84-4.90 (m, 1H), 4.04-4.12 (m, 4H), 3.70-3.75 (m, 2H), 3.13-3.69 (m, 2H), 2.20-2.66 (m, 11H), 1.61-1.65 (m, 6H), 1.45-1.55 (m, 6H), 1.26-1.34 (m, 50H), 0.88 (t, J=6.8 Hz, 9H).

LCMS: (M+H⁺): 945.7 @12.369 minutes.

### 8.31. Synthesis of Compound 2371

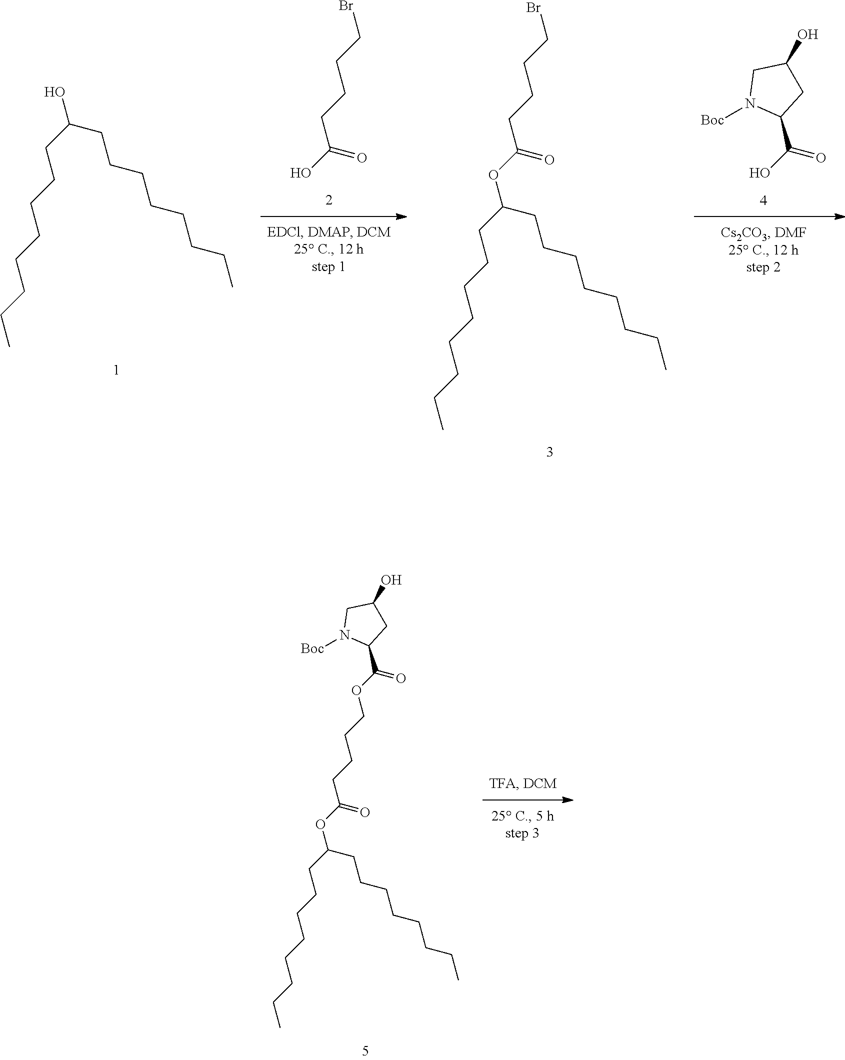 [SMILES: CCCCCCCCC(O)CCCCCCCC ; O=C(O)CCCCBr ; CCCCCCCCC(CCCCCCCC)OC(=O)CCCCBr ; CC(C)(C)OC(=O)N1C[C@@H](O)C[C@H]1C(=O)O ; CCCCCCCCC(CCCCCCCC)OC(=O)CCCCOC(=O)[C@@H]1C[C@H](O)CN1C(=O)OC(C)(C)C]

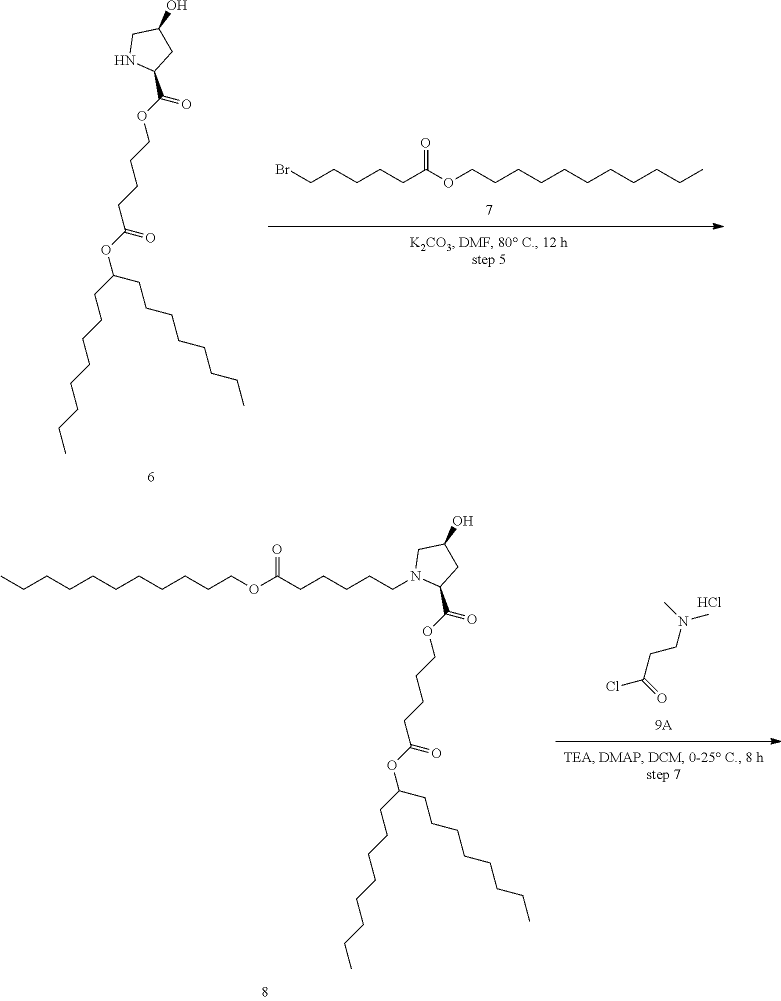

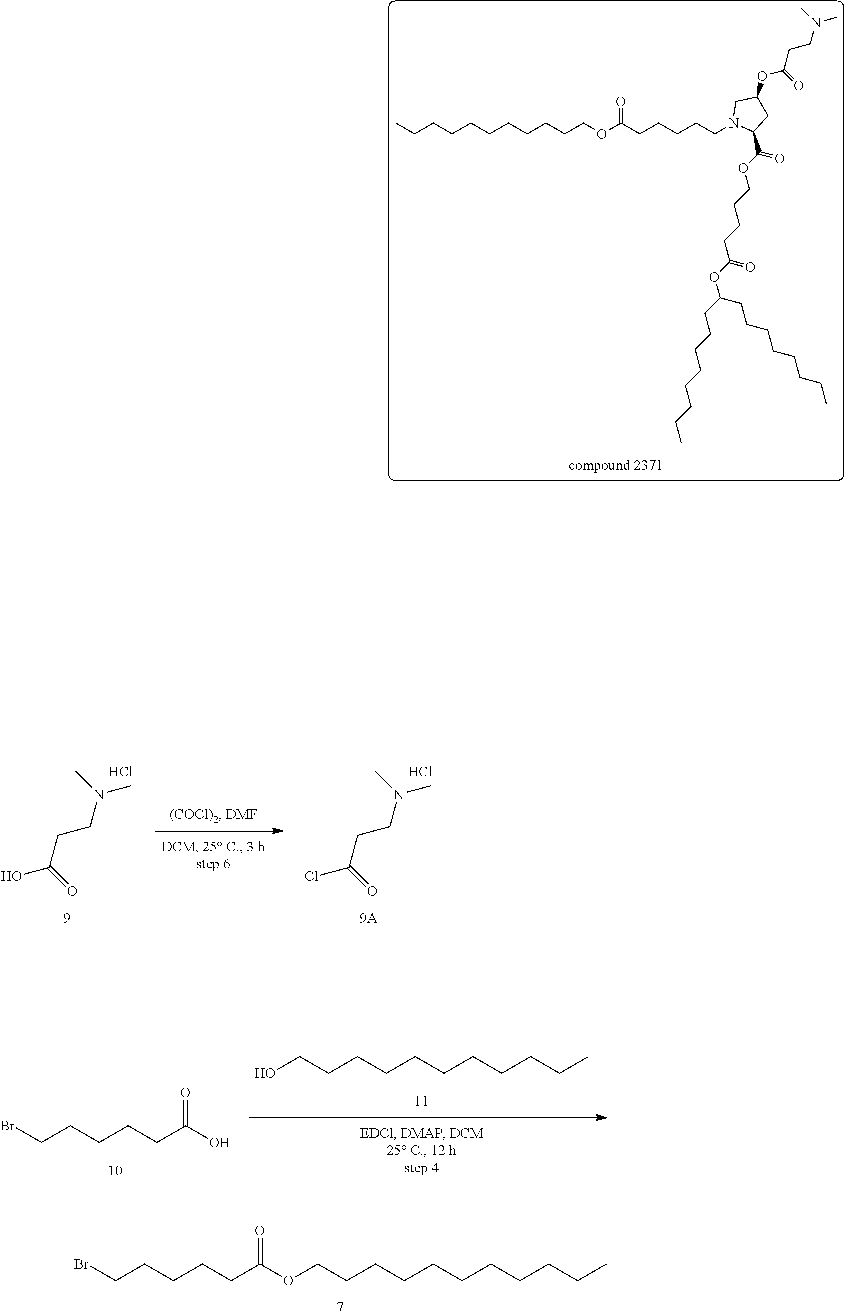

### Step 1

To a solution of heptadecan-9-ol (10 g, 38.99 mmol, 1 eq), 5-bromopentanoic acid (7.06 g, 38.99 mmol, 1 eq), DMAP (952.72 mg, 7.80 mmol, 0.2 eq), EDCI (7.47 g, 38.99 mmol, 1 eq) in DCM (70 mL) was stirred at 25° C. for 12 hours. The combined organic phase was diluted with 200 mL EtOAc and washed with 600 mL water (200 mL×3) and 400 mL brine (200 mL×2), dried with anhydrous Na₂SO₄, filtered, and concentrated under reduced pressure to give a residue. The residue was purified by column chromatography (SiO₂, petroleum ether/ethyl acetate=20/1 to 10/1) to give 1-octylnonyl 5-bromopentanoate (25 g, 59.60 mmol, 76.42% yield) as colorless oil.

¹H NMR (400 MHz, CDCl₃), 4.85-4.91 (m, 1H), 3.42 (t, J=6.8 Hz, 2H), 2.34 (t, J=7.2 Hz, 2H), 1.90-1.93 (m, 2H), 1.79-1.81 (m, 2H), 1.52-1.57 (m, 4H), 1.27-1.51 (m, 24H), 0.89 (t, J=6.4 Hz, 6H).

### Step 2

A mixture of 1-octylnonyl 5-bromopentanoate (2 g, 4.77 mmol, 1 eq), (2S,4S)-1-tert-butoxycarbonyl-4-hydroxy-pyrrolidine-2-carboxylic acid (1.32 g, 5.72 mmol, 1.2 eq), Cs₂CO₃ (3.42 g, 10.49 mmol, 2.2 eq) in DMF (20 mL) was degassed and purged with N₂ for 3 times, and then the mixture was stirred at 25° C. for 12 hours under N₂ atmosphere. The mixture is filtered through celite and the solvent was diluted with 120 mL EtOAc and washed with 150 mL water (50 mL×3) and brine 200 mL (100 mL×2), dried with anhydrous Na₂SO₄, filtered, and concentrated under reduced pressure to give a residue. The residue was purified by column chromatography (SiO₂, petroleum ether/ethyl acetate=10/1 to 8/1, 3% NH₃·H₂O) to give O1-tert-butyl O2-[5-(1-octylnonoxy)-S-oxo-pentyl](2S,4S)-4-hydroxypyrrolidine-1,2-dicarboxylate (1.5 g, 2.58 mmol, 54.08% yield, 98% purity) as yellow oil.

¹H NMR (400 MHz, CDCl₃), 4.81-4.93 (m, 1H), 4.08-4.41 (m, 4H), 3.45-3.68 (m, 2H), 2.11-2.33 (m, 4H), 1.68-1.74 (m, 4H), 1.33-1.51 (m, 13H), 1.25-1.31 (m, 24H), 0.88 (t, J=6.8 Hz, 6H)

### Step 3

To a solution of O1-tert-butyl O2-[5-(1-octylnonoxy)-S-oxo-pentyl](2S,4S)-4-hydroxypyrrolidine-1,2-dicarboxylate (1.50 g, 2.63 mmol, 1 eq) in DCM (20 mL) was added TFA (5.00 g, 43.85 mmol, 3.25 mL, 16.66 eq). The mixture was stirred at 25° C. for 5 hours. The crude reaction mixture was adjusted to pH=7 with aqueous saturated NaHCO₃ and extracted with 180 mL EtOAc (60 mL×3). The combined organic layers were dried over Na₂SO₄, filtered, and concentrated under reduced pressure to give a compound [5-(1-octylnonoxy)-S-oxo-pentyl](2S,4S)-4-hydroxypyrrolidine-2-carboxylate (1 g, 2.04 mmol, 77.64% yield, 96% purity) as yellow oil without purification.

### Step 4

To a mixture of 6-bromohexanoic acid (22.64 g, 116.07 mmol, 1 eq) in DCM (1 mL) was added DMAP (2.84 g, 23.21 mmol, 0.2 eq), undecan-1-ol (20 g, 116.07 mmol, 1 eq), EDCI (22.25 g, 116.07 mmol, 1 eq). The mixture was stirred at 25° C. for 12 hours under N₂ atmosphere. The reaction mixture was diluted with 200 mL H₂O and extracted with 600 mL EtOAc (200 mL×3). The combined organic layers were dried over Na₂SO₄, filtered, and concentrated under reduced pressure to give a residue. The residue was purified by column chromatography (SiO₂, petroleum ether/ethyl acetate=1/0 to 40/1) to give undecyl 6-bromohexanoate (36 g, 103.05 mmol, 88.78% yield) as yellow oil.

### Step 5

To a solution of [5-(1-octylnonoxy)-S-oxo-pentyl](2S,4S)-4-hydroxypyrrolidine-2-carboxylate (1.00 g, 2.13 mmol, 1 eq), undecyl 6-bromohexanoate (892.52 mg, 2.55 mmol, 1.2 eq) in DMF (10 mL) was added K₂CO₃ (882.74 mg, 6.39 mmol, 3 eq). The mixture was stirred at 80° C. for 12 hours. The combined organic phase was diluted with 120 mL EtOAc and washed with 360 mL water (120 mL×3) and 240 mL brine (120 mL×2), dried with anhydrous Na₂SO₄, filtered, and concentrated under reduced pressure to give a residue. The residue was purified by column chromatography (SiO₂, petroleum ether/ethyl acetate=10/1 to 1/1, 3% NH₃·H₂O) to give[5-(1-octylnonoxy)-S-oxo-pentyl](2S,4S)-4-hydroxy-1-(6-oxo-6-undecoxy-hexyl)pyrrolidine-2-carboxylate (0.5 g, 663.84 mol, 31.18% yield, 98% purity) as yellow oil.

¹H NMR (400 MHz, CDCl₃), 4.84-4.88 (m, 1H), 4.23-4.35 (m, 1H), 4.09-4.21 (m, 2H), 4.06 (t, J=6.8 Hz, 2H), 3.21-3.33 (m, 2H), 3.05-3.15 (m, 1H), 2.44-2.75 (m, 3H), 2.25-2.43 (m, 5H), 1.83-2.01 (m, 1H), 1.58-1.63 (m, 6H), 1.42-1.55 (m, 6H), 1.06-1.41 (m, 44H), 0.88 (t, J=6.4 Hz, 9H).

### Step 6

To a solution of 3-(dimethylamino)propanoic acid (0.3 g, 1.95 mmol, 1 eq, HCl), oxalyl dichloride (1.24 g, 9.77 mmol, 854.82 μL, 5 eq), 4A molecular sieve (0.5 g) in DCM (10 mL) was added two drops of DMF (14.27 mg, 195.30 mol, 15.03 μL, 0.1 eq). The mixture was degassed and purged with N₂ for 3 times, and stirred at 25° C. for 3 hours under N₂ atmosphere. The reaction mixture was concentrated under reduced pressure to get compound 3-(dimethylamino)propanoyl chloride (0.3 g, 1.74 mmol, 89.28% yield, HCl) as yellow solid without purification.

### Step 7

To a solution of [5-(1-octylnonoxy)-S-oxo-pentyl](2S,4S)-4-hydroxy-1-(6-oxo-6-undecoxy-hexyl)pyrrolidine-2-carboxylate (0.3 g, 406.43 mol, 1 eq), TEA (370.14 mg, 3.66 mmol, 509.13 μL, 9 eq), DMAP (4.97 mg, 40.64 mol, 0.1 eq) in DCM (3 mL) was added 3-(dimethylamino)propanoyl chloride (279.71 mg, 1.63 mmol, 4 eq, HCl) at 0° C. The mixture was stirred at 25° C. for 8 hours. The combined organic phase was diluted with 60 mL EtOAc and washed with 180 mL water (60 mL×3) and 60 mL brine (30 mL×2), dried with anhydrous Na₂SO₄, filtered, and concentrated under reduced pressure to give a residue. The residue was purified by column chromatography (SiO₂, petroleum ether/ethyl acetate=20/1 to 3/1, 3% NH₃·H₂O) to give [5-(1-octylnonoxy)-S-oxo-pentyl](2S,4S)-4-[3-(dimethylamino)propanoyloxy]-1-(6-oxo-6-undecoxy-hexyl)pyrrolidine-2-carboxylate (0.025 g, 29.86 mol, 33.33% yield, 100% purity) as yellow oil.

¹H NMR (400 MHz, CDCl₃), 5.13-5.39 (m, 1H), 4.81-4.92 (m, 1H), 4.13-4.23 (m, 2H), 4.05 (t, J=6.8 Hz, 2H), 3.21-3.32 (m, 1H), 3.04-3.18 (m, 1H), 2.52-2.84 (m, 7H), 2.25-2.37 (m, 10H), 1.98-2.12 (m, 1H), 1.58-1.63 (m, 5H), 1.45-1.51 (m, 6H), 1.16-1.43 (m, 46H), 0.89 (t, J=6.8 Hz, 9H). LCMS: (M+H⁺): 837.6 @9.196 minutes.

### 8.32. Synthesis of Compound 2372

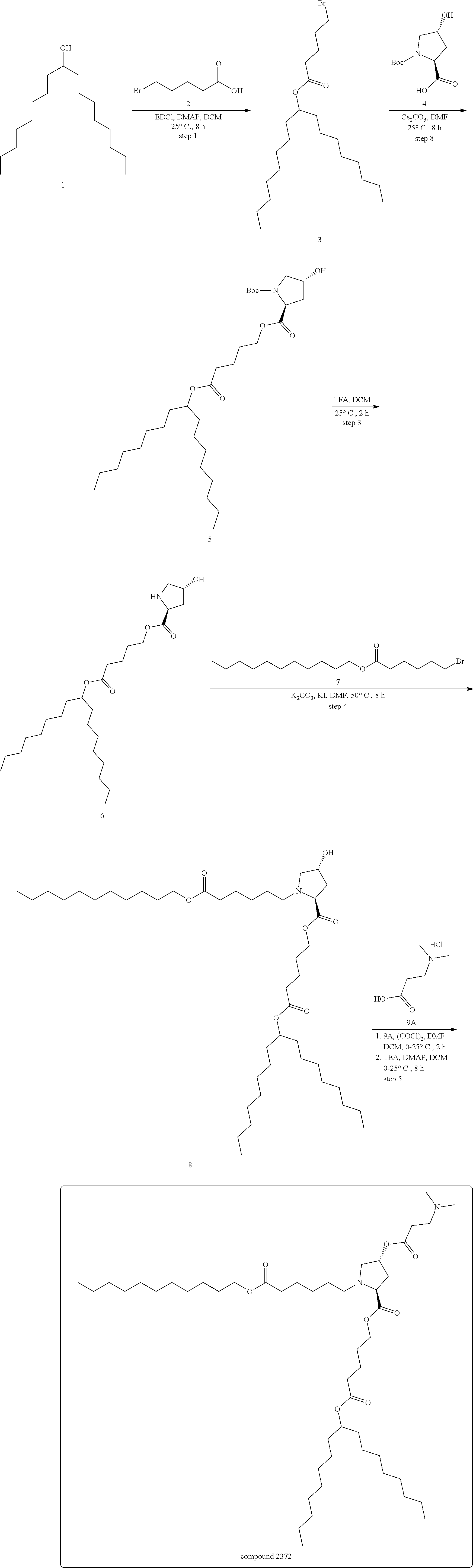

### Step 1

To a solution of 5-bromopentanoic acid (39.03 g, 215.62 mmol, 1.58 eq) in DCM (600 mL) was added EDCI (39.24 g, 204.71 mmol, 1.5 eq) and DMAP (5.00 g, 40.94 mmol, 0.3 eq). The mixture was stirred at 25° C. for 0.5 hour and added heptadecan-9-ol (35 g, 136.47 mmol, 1 eq) at 25° C. The mixture was degassed and purged with N₂ for 3 times, and stirred at 25° C. for 7.5 hours under N₂ atmosphere. The reaction mixture was concentrated under reduced pressure, then diluted with 400 mL H₂O, and extracted with 1500 mL EtOAc (300 mL×5). The combined organic layers were washed with 400 mL brine, dried over Na₂SO₄, filtered, and concentrated under reduced pressure to give a residue. The residue was purified by column chromatography (SiO₂, petroleum ether/ethyl acetate=1/0 to 10/1) to give a compound 1-octylnonyl 5-bromopentanoate (50 g, 119.20 mmol, 87.34% yield) as colorless oil.

### Step 2

A mixture of 1-octylnonyl 5-bromopentanoate (10.88 g, 25.95 mmol, 1.2 eq), Cs₂CO₃ (15.50 g, 47.57 mmol, 2.2 eq) and (2S,4R)-1-tert-butoxycarbonyl-4-hydroxy-pyrrolidine-2-carboxylic acid (5.00 g, 21.62 mmol, 1 eq) in DMF (100 mL) was degassed and purged with N₂ for 3 times, and then the mixture was stirred at 25° C. for 8 hours under N₂ atmosphere. The reaction mixture was added into H₂O (200 mL) and extracted with 150 mL EtOAc (50 mL×3). The organic layer was washed with brine (200 mL), dried over Na₂SO₄, filtered, and concentrated under reduced pressure to give a residue. The residue was purified by column chromatography (SiO₂, petroleum ether/ethyl acetate=5/1 to 0/1, 3% NH₃·H₂O) to give O1-tert-butyl O2-[5-(1-octylnonoxy)-S-oxo-pentyl](2S,4R)-4-hydroxypyrrolidine-1,2-dicarboxylate (9.8 g, 17.20 mmol, 79.54% yield) as colorless oil.

¹H NMR (400 MHz, CDCl₃), 4.85-4.89 (m, 1H), 4.37-4.51 (m, 2H), 4.15-4.30 (m, 2H), 3.46-3.70 (m, 2H), 2.20-2.35 (m, 3H), 2.05-2.15 (m, 1H), 1.62-1.67 (m, 4H), 1.55-1.60 (m, 4H), 1.42 (s, 9H), 1.25-1.32 (m, 24H), 0.89 (t, J=7.2 Hz, 6H).

### Step 3

A mixture of O1-tert-butyl O2-[5-(1-octylnonoxy)-S-oxo-pentyl](2S,4R)-4-hydroxypyrrolidine-1,2-dicarboxylate (9.80 g, 17.20 mmol, 1 eq) in TFA (30.80 g, 270.12 mmol, 20.00 mL, 15.71 eq) and DCM (40 mL) was degassed and purged with N₂ for 3 times, and then the mixture was stirred at 25° C. for 2 hours under N₂ atmosphere. The crude product was concentrated under reduced pressure to get a residue. Then the residue was dissolved with EtOAc (50 mL), and the organic layer was washed with 90 mL aqueous saturated NaHCO₃ (30 mL×3) and 300 mL brine (100 mL×3), dried over Na₂SO₄, filtered, and concentrated under reduced pressure to give [5-(1-octylnonoxy)-S-oxo-pentyl](2S,4R)-4-hydroxypyrrolidine-2-carboxylate (6.8 g, 14.48 mmol, 84.18% yield) as colorless oil.

### Step 4

[5-(1-octylnonoxy)-S-oxo-pentyl](2S,4R)-4-hydroxypyrrolidine-2-carboxylate (6.8 g, 14.48 mmol, 1 eq) was dissolved in DMF (100 mL), KI (1.20 g, 7.24 mmol, 0.5 eq) and K₂CO₃ (6.00 g, 43.43 mmol, 3 eq) were added to the mixture, and undecyl 6-bromohexanoate (5.56 g, 15.93 mmol, 1.1 eq) was added to the reaction mixture. The mixture was stirred for 8 hours at 50° C. The reaction mixture was diluted with 200 mL H₂O and extracted with 270 mL EtOAc (90 mL×3). Then the combined organic layers was washed with 90 mL brine (30 mL×3). The combined organic layers were dried over Na₂SO₄, filtered, and concentrated under reduced pressure to give a residue. The residue was purified by column chromatography (SiO₂, petroleum ether/ethyl acetate=5/1 to 0/1, 3% NH₃·H₂O) to give a compound [5-(1-octylnonoxy)-S-oxo-pentyl](2S,4R)-4-hydroxy-1-(6-oxo-6-undecoxy-hexyl)pyrrolidine-2-carboxylate (1 g, 1.35 mmol, 9.36% yield,—purity) as colorless oil.

1H NMR (400 MHz, CDCl₃), 4.85-4.89 (m, 1H), 4.49-4.51 (m, 1H), 4.01-4.25 (m, 4H), 3.34-3.83 (m, 2H), 2.51-2.83 (m, 2H), 2.29-2.34 (m, 5H), 1.71-1.83 (m, 16H), 1.15-1.32 (m, 42H), 0.89 (t, J=6.8 Hz, 9H).

### Step 5

A mixture of 3-(dimethylamino)propanoic acid (550 mg, 3.58 mmol, 1 eq, HCl) in DCM (100 mL) was added (COCl)₂ (2.27 g, 17.90 mmol, 1.57 mL, 5 eq) and DMF (13.09 mg, 179.03 mol, 13.77 μL, 0.05 eq) dropwise at 0° C. under N₂ atmosphere. The mixture was degassed and purged with N₂ for 3 times, and then stirred at 25° C. for 2 hours under N₂ atmosphere. The reaction mixture was concentrated under reduced pressure to give 3-(dimethylamino)propanoyl chloride (583 mg, 3.39 mmol, 94.64% yield, HCl) as yellow solid. The crude oil residue was dissolved with DCM (10 mL), and then the mixture was added into a solution of [5-(1-octylnonoxy)-S-oxo-pentyl](2S,4R)-4-hydroxy-1-(6-oxo-6-undecoxy-hexyl) pyrrolidine-2-carboxylate (500 mg, 677.39 mol, 1 eq), TEA (479.81 mg, 4.74 mmol, 659.99 μL, 7 eq) and DMAP (41.38 mg, 338.69 mol, 0.5 eq) in DCM (10 mL) at 0° C. The mixture was stirred at 25° C. for 8 hours. The crude reaction mixture was quenched with aqueous saturated NaHCO₃ and extracted with 300 mL DCM (100 mL×3). The combined organic layers were dried over Na₂SO₄, filtered, and concentrated under reduced pressure to give a residue. The residue was purified by column chromatography (SiO₂, petroleum ether/ethyl acetate=10/1 to 0/1, 3% NH₃·H₂O) to give a compound [5-(1-octylnonoxy)-S-oxo-pentyl](2S,4R)-4-[3-(dimethylamino)propanoyloxy]-1-(6-oxo-6-undecoxy-hexyl)pyrrolidine-2-carboxylate (98 mg, 117.05 mol, 17.28% yield) as yellow oil.

¹H NMR (400 MHz, CDCl₃), 5.26-5.28 (m, 1H), 4.86-4.89 (m, 1H), 4.04-4.14 (m, 4H), 3.44-3.55 (m, 2H), 2.12-2.90 (m, 17H), 1.70-1.80 (m, 4H), 1.60-1.66 (m, 4H), 1.45-1.55 (m, 6H), 1.15-1.40 (m, 44H), 0.89 (t, J=6.4 Hz, 9H). LCMS: (M+H⁺): 837.6 @9.227 minutes

### 8.33. Synthesis of Compound 2373

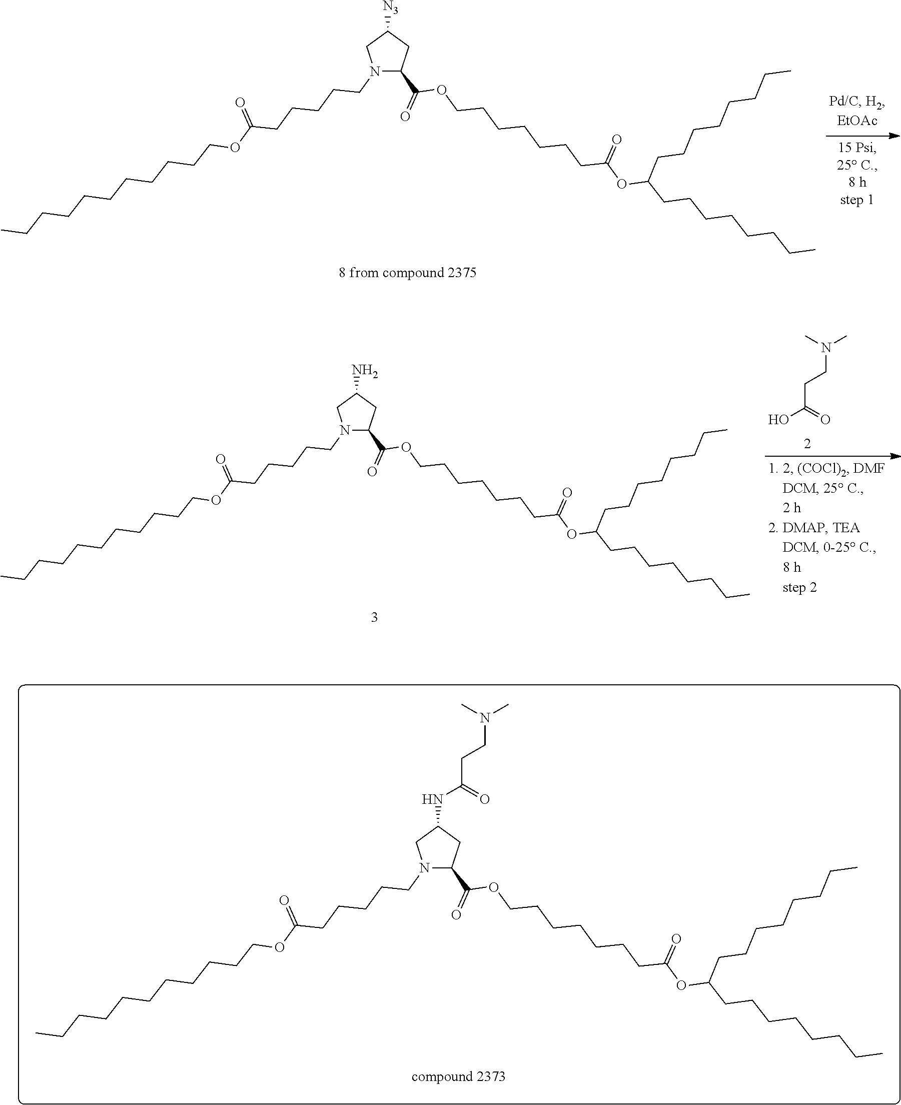

### Step 1

To a solution of Pd/C (0.5 g, 2.36 mmol, 10% purity, 1 eq) in EtOAc (20 mL) was added [8-(1-octylnonoxy)-8-oxo-octyl](2S,4R)-4-azido-1-(6-oxo-6-undecoxy-hexyl)pyrrolidine-2-carboxylate (1.9 g, 2.36 mmol, 1 eq). The mixture was stirred at 25° C. for 8 hours under 15 Psi under H₂. The mixture was filtered and concentrated under reduced pressure to give [8-(1-octylnonoxy)-8-oxo-octyl](2S,4R)-4-amino-1-(6-oxo-6-undecoxy-hexyl)pyrrolidine-2-carboxylate (1.7 g, crude) as yellow oil.

### Step 2

To a solution of 3-(dimethylamino)propanoic acid (430 mg, 2.80 mmol, 1 eq, HCl) in DCM (5 mL) was added (COCl)₂ (1.42 g, 11.20 mmol, 980.20 μL, 4 eq) and DMF (20.46 mg, 279.94 mol, 21.54 μL, 0.1 eq). The mixture was stirred at 25° C. for 2 hours. The mixture was concentrated under reduced pressure to give 3-(dimethylamino)propanoyl chloride (2.4 g, crude, HCl) as a yellow solid. To a solution of [8-(1-octylnonoxy)-8-oxo-octyl](2S,4R)-4-amino-1-(6-oxo-6-undecoxy-hexyl)pyrrolidine-2-carboxylate (500 mg, 641.66 mol, 1 eq) and TEA (324.65 mg, 3.21 mmol, 446.56 μL, 5 eq) in DCM (10 mL) was added DMAP (39.20 mg, 320.83 mol, 0.5 eq) and 3-(dimethylamino)propanoyl chloride (473.08 mg, 2.75 mmol, 4.29 eq, HCl) under N₂ at 0° C., and then the mixture was stirred at 25° C. for 8 hours. The mixture was added into saturated NaHCO₃ (20 mL), and extracted with EtOAc (10 mL×3). The organic layer was washed with brine (10 mL×2), dried over Na₂SO₄, filtered, and concentrated under reduced pressure. The residue was purified by column chromatography (SiO₂, petroleum ether/ethyl acetate=10/1 to 0/1) and prep-TLC (SiO₂, Ethyl acetate/MeOH=5:1, added 3% NH₃·H₂O) to give [8-(1-octylnonoxy)-8-oxo-octyl](2S,4R)-4-[3-(dimethylamino)propanoyl amino]-1-(6-oxo-6-undecoxy-hexyl)pyrrolidine-2-carboxylate (20 mg, 54.65 mol, 8.52% yield, 97.5 purity) as yellow oil.

1H NMR (400 MHz, CDCl₃), 8.32-8.41 (m, 1H), 4.80-4.90 (m, 1H), 4.46-4.50 (m, 1H), 4.03-4.15 (m, 4H), 3.38-3.47 (m, 2H), 2.55-2.75 (m, 3H), 2.25-2.45 (m, 15H), 1.91-1.95 (m, 1H), 1.59-1.68 (m, 8H), 1.45-1.56 (m, 6H), 1.20-1.40 (m, 48H), 0.89 (t, J=6.8 Hz, 9H).

LCMS: (M+H⁺): 878.8 @10.175 minutes.

### 8.34. Synthesis of Compound 2375

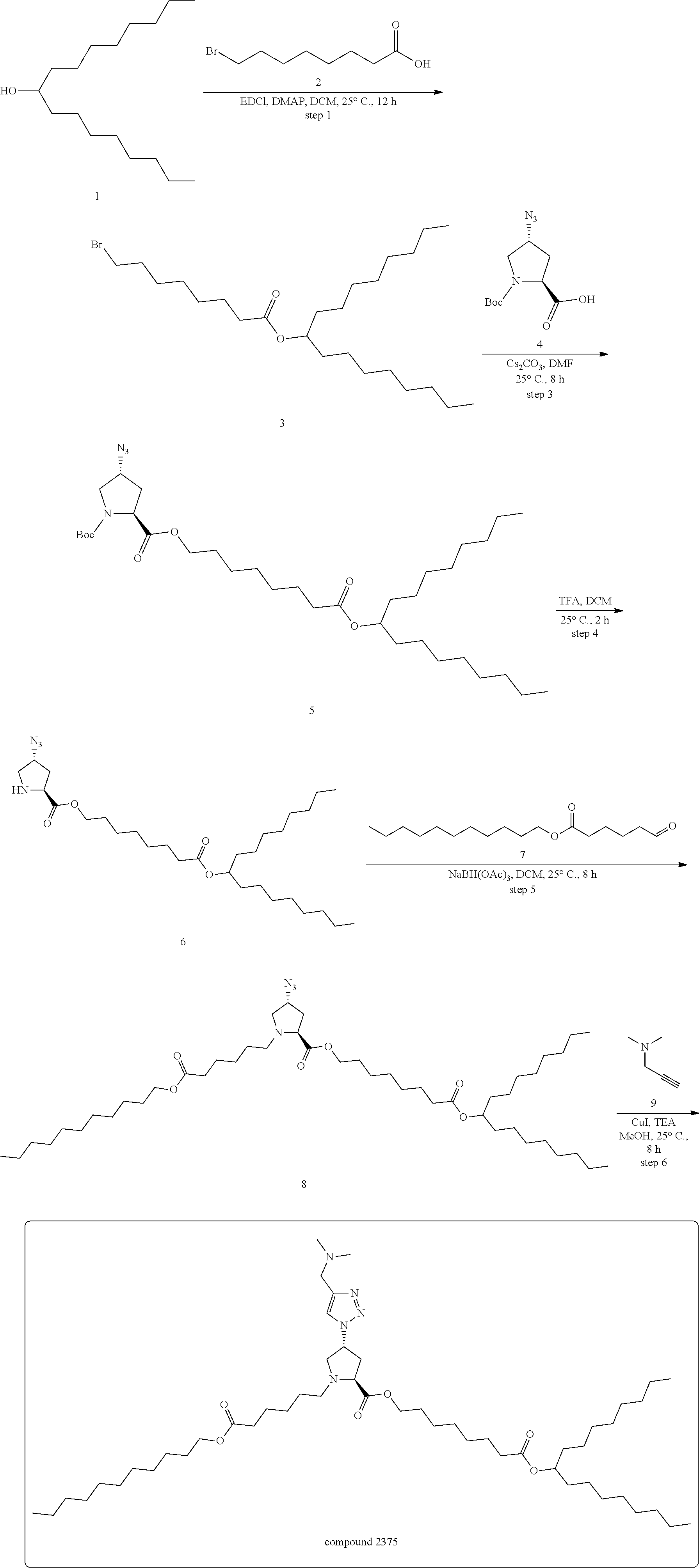

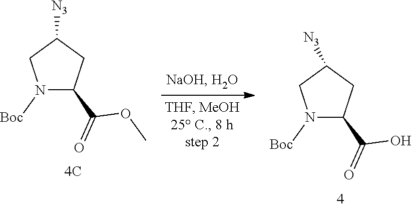 [SMILES: COC(=O)[C@@H]1C[C@@H](N=[N+]=[N-])CN1C(=O)OC(C)(C)C ; CC(C)(C)OC(=O)N1C[C@H](N=[N+]=[N-])C[C@H]1C(=O)O]

### Step 1

To a mixture of 8-bromooctanoic acid (36.00 g, 161.36 mmol, 2.25 eq), EDCI (27.50 g, 143.43 mmol, 2 eq), DMAP (3.50 g, 28.69 mmol, 0.4 eq) in DCM (300 mL) was added heptadecan-9-ol (18.39 g, 71.71 mmol, 1 eq), and degassed and purged with N₂ for 3 times, and then the mixture was stirred at 25° C. for 8 hours under N₂ atmosphere. The reaction mixture was diluted with 200 mL H₂O and extracted with 200 mL EtOAc (100 mL×2). The combined organic layers were dried over Na₂SO₄, filtered, and concentrated under reduced pressure to give a residue. The residue was purified by column chromatography (SiO₂, petroleum ether/ethyl acetate=1/0 to 20/1) to give 1-octylnonyl 8-bromooctanoate (32.4 g, 70.20 mmol, 97.88% yield) as colorless oil.

### Step 2

To a solution of O1-tert-butyl O2-methyl (2S,4R)-4-azidopyrrolidine-1,2-dicarboxylate (2 g, 7.40 mmol, 1 eq) in THF (20 mL) and MeOH (10 mL) was added a solution of NaOH (1.78 g, 44.40 mmol, 6 eq) in H₂O (7.39 g, 410.48 mmol, 7.39 mL, 55.47 eq). The mixture was stirred at 25° C. for 8 hours. The mixture was adjusted to pH=3 with 1N HCl, and then extracted with 30 mL EtOAc (10 mL×3). The combined organic layers were washed with 30 mL saturated brine (10 mL×3), dried over Na₂SO₄, filtered, and concentrated under reduced pressure to give (2S,4R)-4-azido-1-tert-butoxycarbonyl-pyrrolidine-2-carboxylic acid (1.9 g, crude) as colorless oil used into the next step without further purification.

### Step 3

To a solution of (2S,4R)-4-azido-1-tert-butoxycarbonyl-pyrrolidine-2-carboxylic acid (1.9 g, 7.41 mmol, 1 eq) and 1-octylnonyl 8-bromooctanoate (4.11 g, 8.90 mmol, 1.2 eq) in DMF (100 mL) was added Cs₂CO₃ (5.31 g, 16.31 mmol, 2.2 eq). The mixture was stirred at 25° C. for 8 hours. The mixture was added into H₂O (200 mL), and extracted with EtOAc (200 mL×3). The organic layer was washed with brine (200 mL×2), dried over Na₂SO₄, filtered, and the filtrate was concentrated under reduced pressure. The residue was purified by column chromatography (SiO₂, petroleum ether/ethyl acetate=1/0 to 5/1) to give O1-tert-butyl O2-[8-(1-octylnonoxy)-8-oxo-octyl](2S,4R)-4-azidopyrrolidine-1,2-dicarboxylate (4 g, 6.28 mmol, 84.71% yield) as yellow oil.

¹H NMR (400 MHz, CDCl₃), 4.85-4.90 (m, 1H), 4.30-4.40 (m, 1H), 4.05-4.25 (m, 3H), 3.45-3.75 (m, 2H), 2.10-2.45 (m, 4H), 1.60-1.70 (m, 4H), 1.57 (s, 3H), 1.40-1.55 (m, 13H), 1.20-1.40 (m, 30H), 0.88 (t, J=6.0 Hz, 6H).

### Step 4

To a solution of O1-tert-butyl O2-[8-(1-octylnonoxy)-8-oxo-octyl](2S,4R)-4-azidopyrrolidine-1,2-dicarboxylate (2 g, 3.14 mmol, 1 eq) in DCM (10 mL) was added TFA (30.80 g, 270.12 mmol, 20.00 mL, 86.02 eq). The mixture was stirred at 25° C. for 2 hours. The mixture was added into saturated NaHCO₃ (200 mL), and extracted with EtOAc (100 mL×3). The organic layer was washed with brine (50 mL×2), dried over Na₂SO₄, filtered, and the filtrate was concentrated under reduced pressure to give [8-(1-octylnonoxy)-8-oxo-octyl](2S,4R)-4-azidopyrrolidine-2-carboxylate (1.7 g, crude) as yellow oil.

### Step 5

To a solution of [8-(1-octylnonoxy)-8-oxo-octyl](2S,4R)-4-azidopyrrolidine-2-carboxylate (1.7 g, 3.17 mmol, 1 eq) and undecyl 6-oxohexanoate (1.08 g, 3.80 mmol, 1.2 eq) in DCM (20 mL) was added NaBH(OAc)₃ (2.01 g, 9.50 mmol, 3 eq). The mixture was stirred at 25° C. for 8 hours. The mixture was added into H₂O (20 mL), and extracted with EtOAc (20 mL×3). The organic layer was washed with brine (20 mL×2), dried over Na₂SO₄, filtered, and the filtrate was concentrated under reduced pressure. The residue was purified by column chromatography (SiO₂, petroleum ether/ethyl acetate=1/0 to 5/1) to give [8-(1-octylnonoxy)-8-oxo-octyl](2S,4R)-4-azido-1-(6-oxo-6-undecoxy-hexyl) pyrrolidine-2-carboxylate (2 g, 2.48 mmol, 78.43% yield,—purity) was obtained as yellow oil.

1H NMR (400 MHz, CDCl₃), 4.85-4.90 (m, 1H), 4.05-4.25 (m, 5H), 3.45-3.48 (m, 2H), 2.40-2.75 (m, 3H), 2.15-2.40 (m, 6H), 1.45-1.75 (m, 16H), 1.20-1.40 (m, 49H), 0.88 (t, J=6.0 Hz, 9H).

### Step 6

To a solution of [8-(1-octylnonoxy)-8-oxo-octyl](2S,4R)-4-azido-1-(6-oxo-6-undecoxy-hexyl)pyrrolidine-2-carboxylate (150 mg, 186.28 mol, 1 eq), CuI (3.55 mg, 18.63 mol, 0.1 eq) and TEA (1.88 mg, 18.63 mol, 2.59 μL, 0.1 eq) in MeOH (5 mL) was added N,N-dimethylprop-2-yn-1-amine (18.58 mg, 223.54 mol, 23.70 μL, 1.2 eq). The mixture was stirred at 25° C. for 8 hours. The mixture was added into H₂O (20 mL), and extracted with EtOAc (20 mL×3). The organic layer was washed with brine (20 mL×2), dried over Na₂SO₄, filtered, and the filtrate was concentrated under reduced pressure. The residue was purified by prep-TLC (SiO₂, ethyl acetate/MeOH=10/1, added 3% NH₃·H₂O) and purified by column chromatography (SiO₂, petroleum ether/ethyl acetate=1/0 to 5/1) to give [8-(1-octylnonoxy)-8-oxo-octyl](2S,4R)-4-[4-[(dimethylamino)methyl]triazol-1-yl]-1-(6-oxo-6-undecoxy-hexyl)pyrrolidine-2-carboxylate (120 mg, 168.85 mol, 90.64% yield, 99% purity) as yellow oil.

¹H NMR (400 MHz, CDCl₃), 7.62 (s, 1H), 5.26-5.32 (m, 1H), 4.80-4.90 (m, 1H), 4.03-4.15 (m, 4H), 3.70-3.74 (m, 1H), 3.56-3.62 (m, 3H), 2.90-3.00 (m, 1H), 2.45-2.80 (m, 4H), 2.25-2.65 (m, 10H), 1.59-1.68 (m, 8H), 1.47-1.56 (m, 6H), 1.26-1.40 (m, 48H), 0.88 (t, J=6.4 Hz, 9H).

LCMS: (M+H⁺): 888.4 @13.974 minutes.

### 8.35. Synthesis of Compound 2376

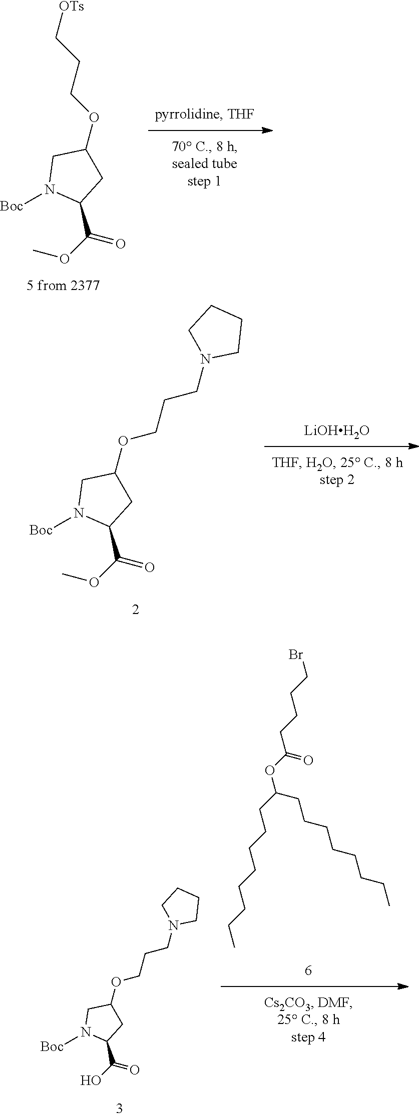 [SMILES: COC(=O)[C@@H]1CC(OCCCOS(=O)(=O)c2ccc(C)cc2)CN1C(=O)OC(C)(C)C ; COC(=O)[C@@H]1CC(OCCCN2CCCC2)CN1C(=O)OC(C)(C)C ; "LiOH·H2O" ; CC(C)(C)OC(=O)N1CC(OCCCN2CCCC2)C[C@H]1C(=O)O ; CCCCCCCCC(CCCCCCCC)OC(=O)CCCCBr]

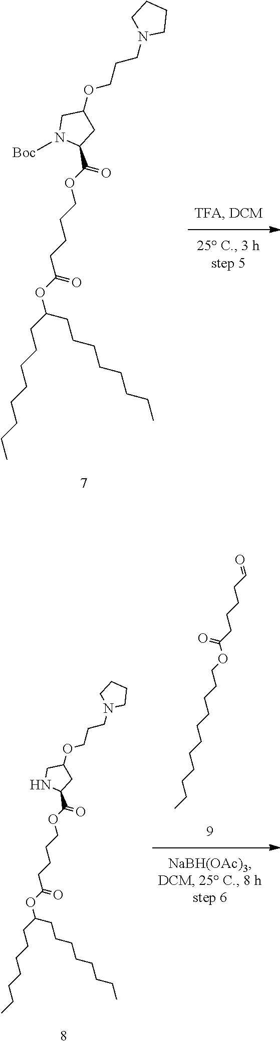 [SMILES: CCCCCCCCC(CCCCCCCC)OC(=O)CCCCOC(=O)[C@@H]1CC(OCCCN2CCCC2)CN1C(=O)OC(C)(C)C ; CCCCCCCCC(CCCCCCC)OC(=O)CCCCOC(=O)[C@@H]1CC(OCCCN2CCCC2)CN1 ; CCCCCCCCCCCOC(=O)CCCCC=O]

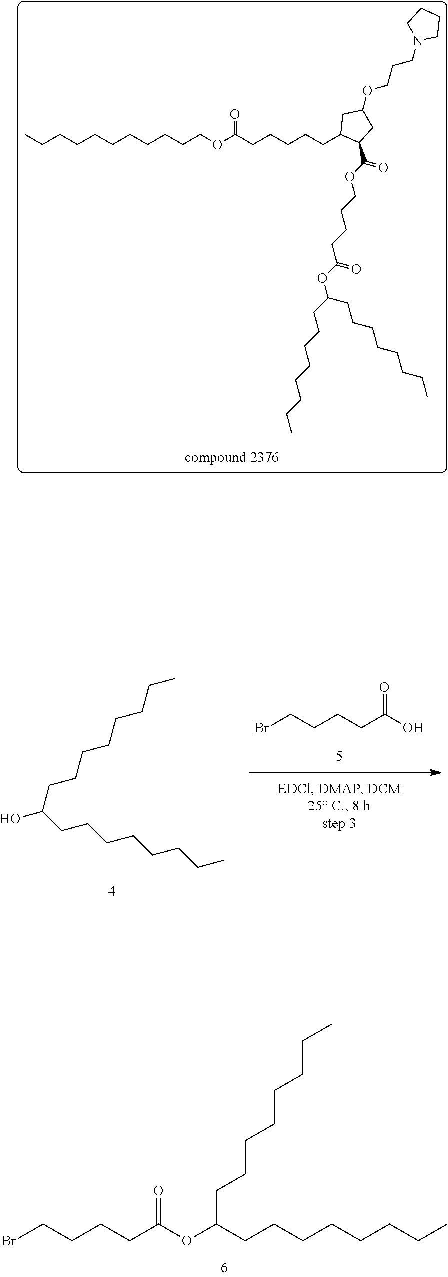 [SMILES: CCCCCCCCCCCOC(=O)CCCCCC1CC(OCCCN2CCCC2)C[C@H]1C(=O)OCCCCC(=O)OC(CCCCCCCC)CCCCCCCC ; CCCCCCCCC(O)CCCCCCCC ; O=C(O)CCCCBr ; CCCCCCCCC(CCCCCCCC)OC(=O)CCCCBr]

### Step 1

To a solution of O1-tert-butyl O2-methyl (2S)-4-[3-(p-tolylsulfonyloxy)propoxy]pyrrolidine-1,2-dicarboxylate (6 g, 13.11 mmol, 1 eq) in THF (60 mL) was added pyrrolidine (932.65 mg, 13.11 mmol, 1.09 mL, 1 eq). The mixture was stirred at 70° C. for 8 hours in sealed tube. The reaction mixture was quenched by addition of 20 mL H₂O at 0° C., and then extracted with 60 mL EtOAc (20 mL×3). The combined organic layers were dried over Na₂SO₄, filtered, and concentrated under reduced pressure to give a residue. The residue was purified by column chromatography (SiO₂, petroleum ether/ethyl acetate=10/1 to 0/1, added 0.5% NH₃·H₂O) to give O1-tert-butyl O2-methyl (2S)-4-(3-pyrrolidin-1-ylpropoxy)pyrrolidine-1,2-dicarboxylate (2.1 g, 5.89 mmol, 44.93% yield) as yellow oil.

### Step 2

To a solution of O1-tert-butyl O2-methyl (2S)-4-(3-pyrrolidin-1-ylpropoxy)pyrrolidine-1,2 dicarboxylate (2.1 g, 5.89 mmol, 1 eq) in THF (10 mL) was added LiOH·H₂O (494.44 mg, 11.78 mmol, 2 eq) and H₂O (10 mL). The mixture was stirred at 25° C. for 8 hours. The reaction mixture was diluted with 20 mL H₂O and extracted with 30 mL EtOAc (10 mL×3). The aqueous layers were concentrated under reduced pressure to give (2S)-1-tert-butoxycarbonyl-4-(3-pyrrolidin-1-ylpropoxy)pyrrolidine-2-carboxylic acid (2 g, crude, Li⁺ salt) as yellow oil.

### Step 3

To a solution of heptadecan-9-ol (5 g, 19.50 mmol, 1 eq) in DCM (100 mL) was added EDCI (5.61 g, 29.24 mmol, 1.5 eq), DMAP (714.53 mg, 5.85 mmol, 0.3 eq) and 5-bromopentanoic acid (5.58 g, 30.80 mmol, 1.58 eq). The mixture was stirred at 25° C. for 8 hours under N₂ atmosphere. The reaction mixture was quenched by addition of 100 mL H₂O at 0° C., and then extracted with 150 mL EtOAc (50 mL×3). The combined organic layers were dried over Na₂SO₄, filtered, and concentrated under reduced pressure to give a residue. The residue was purified by column chromatography (SiO₂, petroleum ether/ethyl acetate=10/1 to 0/1) to give 1-octylnonyl 5-bromopentanoate (12 g, 28.61 mmol, 73.37% yield) as yellow oil.

1H NMR (400 MHz, CDCl₃), 4.85-4.89 (m, 1H), 3.42 (t, J=6.8 Hz, 2H), 2.33 (t, J=7.2 Hz, 2H), 1.77-1.93 (m, 4H), 1.51-1.56 (m, 4H), 1.25-1.45 (m, 24H), 0.88 (t, J=5.6 Hz, 6H)

### Step 4

To a solution of (2S)-1-tert-butoxycarbonyl-4-(3-pyrrolidin-1-ylpropoxy)pyrrolidine-2-carboxylic acid (2 g, 5.84 mmol, 1 eq) in DMF (20 mL) was added Cs₂CO₃ (4.19 g, 12.85 mmol, 2.2 eq) and 1-octylnonyl 5-bromopentanoate (2.94 g, 7.01 mmol, 1.2 eq). The mixture was stirred at 25° C. for 8 hours. The reaction mixture was quenched by addition of 20 mL H₂O at 0° C., and then extracted with 60 mL EtOAc (20 mL×3). The combined organic layers were dried over Na₂SO₄, filtered, and concentrated under reduced pressure to give a residue. The residue was purified by chromatography (SiO₂, petroleum ether/ethyl acetate=1/0 to 0/1, added 0.5% NH₃·H₂O) to give O1-tert-butyl O2-[5-(1-octylnonoxy)-S-oxo-pentyl](2S)-4-(3-pyrrolidin-1-ylpropoxy)pyrrolidine-1,2-dicarboxylate (1.8 g, 2.64 mmol, 45.26% yield) as yellow oil.

¹H NMR (400 MHz, CDCl₃), 4.85-4.88 (m, 1H), 4.05-4.33 (m, 4H), 3.43-3.67 (m, 3H), 2.32-2.61 (m, 8H), 1.99-2.18 (m, 1H), 1.60-1.80 (m, 14H), 1.42-1.51 (m, 13H), 1.26-1.41 (m, 25H), 0.88 (t, J=6.4 Hz, 6H).

### Step 5

To a solution of O1-tert-butyl O2-[5-(1-octylnonoxy)-S-oxo-pentyl](2S)-4-(3-pyrrolidin-1-ylpropoxy)pyrrolidine-1,2-dicarboxylate (1.8 g, 2.64 mmol, 1 eq) in DCM (16 mL) was added TFA (8 mL). The mixture was stirred at 25° C. for 3 hours. The mixture was concentrated under reduced pressure, then adjusted to pH=8 with saturated NaHCO₃, and extracted with 90 mL EtOAc (30 mL×3). The combined organic layers were dried over Na₂SO₄, filtered, and concentrated under reduced pressure to give a residue. The residue was purified by column chromatography (SiO₂, petroleum ether/ethyl acetate=1/0 to 1/1) to give [5-(1-octylnonoxy)-S-oxo-pentyl](2S)-4-(3-pyrrolidin-1-ylpropoxy)pyrrolidine-2-carboxylate (1.5 g, 2.58 mmol, 97.70% yield) as yellow oil.

¹H NMR (400 MHz, CDCl₃), 4.85-4.88 (m, 1H), 3.90-4.17 (m, 4H), 3.75-3.80 (m, 1H), 3.42-3.43 (m, 2H), 3.05-3.22 (m, 7H), 2.85-2.86 (m, 1H), 2.18-2.33 (m, 6H), 1.99-2.07 (m, 8H), 1.50-1.52 (m, 4H), 1.26-1.31 (m, 25H), 0.88 (t, J=6.4 Hz, 6H).

### Step 6

To a solution of [5-(1-octylnonoxy)-S-oxo-pentyl](2S)-4-(3-pyrrolidin-1-ylpropoxy)pyrrolidine-2-carboxylate (500 mg, 860.76 mol, 1 eq) in DCM (5 mL) was added undecyl 6-oxohexanoate (293.80 mg, 1.03 mmol, 1.2 eq) and NaBH(OAc)₃ (547.29 mg, 2.58 mmol, 3 eq). The mixture was stirred at 25° C. for 8 hours. The reaction mixture was quenched by addition of 20 mL H₂O at 0° C., and then extracted with 60 mL EtOAc (20 mL×3). The combined organic layers were dried over Na₂SO₄, filtered, and concentrated under reduced pressure to give a residue. The residue was purified by prep-TLC (SiO₂, petroleum ether/ethyl acetate=0/1, added 0.5% NH₃·H₂O) to give [5-(1-octylnonoxy)-S-oxo-pentyl](2S)-1-(6-oxo-6-undecoxy-hexyl)-4-(3-pyrrolidin-1-ylpropoxy)-2-carboxylate (200 mg, 235.48 mol, 27.36% yield) as yellow oil.

¹H NMR (400 MHz, CDCl₃), 4.85-4.88 (m, 1H), 3.99-4.14 (m, 5H), 3.42 (t, J=6.8 Hz, 2H), 3.08-3.23 (m, 2H), 2.27-2.54 (m, 15H), 1.98-2.01 (m, 1H), 1.70-1.81 (m, 5H), 1.65-1.69 (m, 4H), 1.61-1.63 (m, 4H), 1.50-1.52 (m, 6H), 1.26-1.34 (m, 41H), 0.88 (t, J=6.4 Hz, 9H).

LCMS (CAD): (M+H⁺): 847.2 @8.653 minutes.

### 8.36. Synthesis of Compound 2377

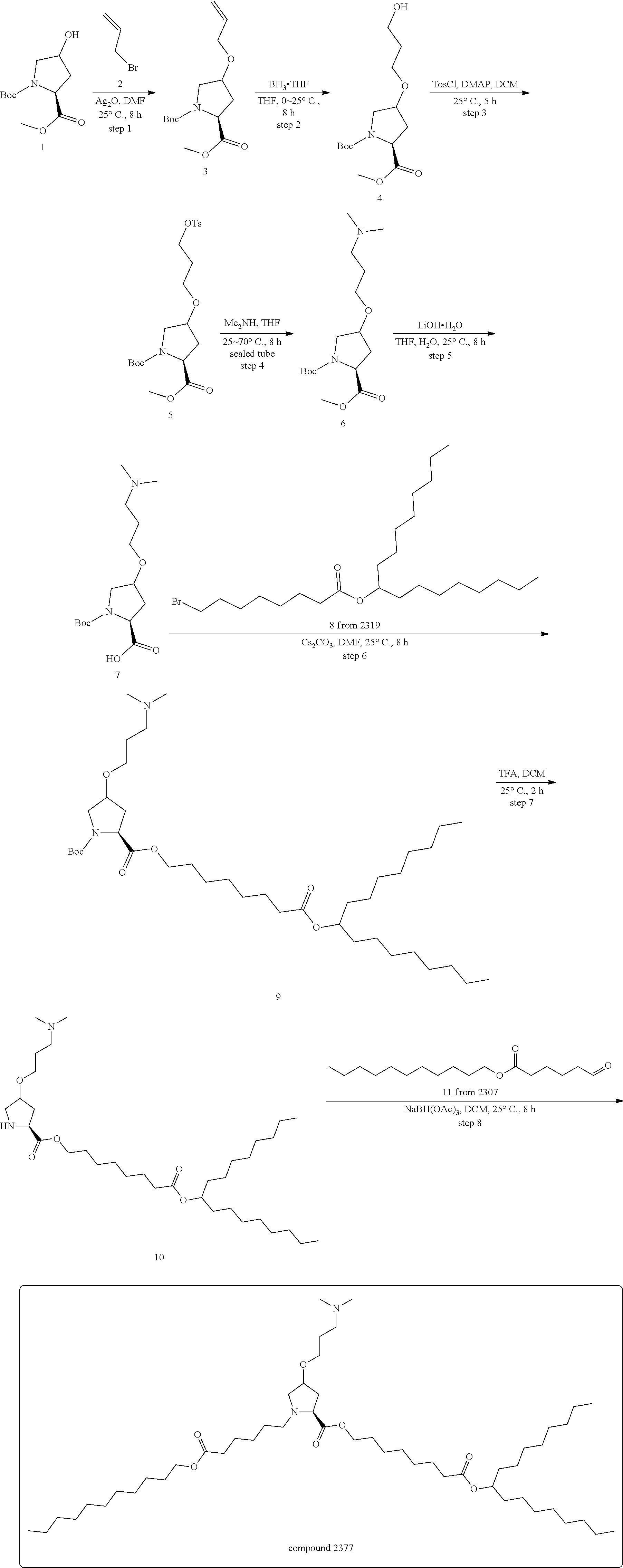

### Step 1

To a solution of O1-tert-butyl O2-methyl (2S)-4-hydroxypyrrolidine-1,2-dicarboxylate (10 g, 40.77 mmol, 1 eq) in DMF (100 mL) was added Ag₂O (14.17 g, 61.16 mmol, 1.5 eq), 3-bromoprop-1-ene (8.88 g, 73.39 mmol, 1.8 eq) in dark. The mixture was stirred at 25° C. for 8 hours. The reaction mixture was filtered and concentrated under reduced pressure to give a residue. The residue was purified by column chromatography (SiO₂, petroleum ether/ethyl acetate=1/0 to 3/1) to give O1-tert-butyl O2-methyl (2S)-4-allyloxypyrrolidine-1,2-dicarboxylate (6 g, 21.03 mmol, 51.58% yield) as yellow oil.

¹H NMR (400 MHz, CDCl₃), 5.78-5.99 (m, 1H), 5.12-5.30 (m, 2H), 4.38-4.49 (m, 1H), 4.03-4.20 (m, 1H), 3.87-4.02 (m, 2H), 3.68-3.78 (m, 3H), 3.43-3.67 (m, 2H), 2.01-2.44 (m, 2H), 1.40-1.51 (m, 9H).

### Step 2

To a solution of O1-tert-butyl O2-methyl (2S)-4-allyloxypyrrolidine-1,2-dicarboxylate (10 g, 35.05 mmol, 1 eq) in THF (300 mL) was added a solution of BH₃·THF (1 M, 12.62 mL, 0.36 eq) at 0° C. in N₂ atmosphere. The reaction liquid was warmed to 25° C. and stirred for 8 hours. The mixture was cooled to 0° C., and then aqueous saturated NH₄C₁ (100 mL) was added to the mixture under N₂ atmosphere. The mixture was stirred for 10 minutes, and then extracted with 300 mL EtOAc (100 mL×3). The combined organic layers were dried over Na₂SO₄, filtered, and concentrated under reduced pressure to give a residue. The residue was purified by column chromatography (SiO₂, petroleum ether/ethyl acetate=20/1 to 0/1, 3% NH₃·H₂O) to give O1-tert-butyl O2-methyl (2S)-4-(3-hydroxypropoxy)pyrrolidine-1,2-dicarboxylate (12 g, 39.56 mmol, 28.22% yield, 100% purity) as yellow oil.

¹H NMR (400 MHz, CDCl₃), 4.28-4.53 (m, 1H), 3.33-4.09 (m, 10H), 2.15-2.40 (m, 2H), 1.73-1.84 (m, 2H), 1.39-1.51 (m, 9H).

### Step 3

To a solution of O1-tert-butyl O2-methyl (2S)-4-(3-hydroxypropoxy)pyrrolidine-1,2-dicarboxylate (12 g, 39.56 mmol, 1 eq) in DCM (50 mL) was added DMAP (9.67 g, 79.12 mmol, 2 eq) and TosCl (11.31 g, 59.34 mmol, 1.5 eq). The mixture was stirred at 25° C. for 5 hours. The combined organic phase was diluted with 200 mL EtOAc and washed with 600 mL water (200 mL×3) and 400 mL brine (200 mL×2), dried with anhydrous Na₂SO₄, filtered, and concentrated under reduced pressure to give a residue. The residue was purified by column chromatography (SiO₂, petroleum ether/ethyl acetate=1/1 to 0/1) to give O1-tert-butyl 02-methyl (2S)-4-[3-(p-tolylsulfonyloxy) propoxy]pyrrolidine-1,2-dicarboxylate (7.2 g, 15.74 mmol, 39.78% yield) as yellow oil.

¹H NMR (400 MHz, CDCl₃), 7.65-7.78 (m, 2H), 7.24-7.35 (m, 2H), 3.76-4.39 (m, 4H), 3.58-3.70 (m, 3H), 3.24-3.54 (m, 4H), 2.33-2.45 (s, 3H), 2.03-2.26 (m, 2H), 1.87-1.98 (m, 1H), 1.69-1.83 (m, 2H), 1.37-1.45 (m, 9H).

### Step 4

To a solution of O1-tert-butyl O2-methyl (2S)-4-[3-(p-tolylsulfonyloxy)propoxy]pyrrolidine-1,2-dicarboxylate (4 g, 8.74 mmol, 1 eq) in THF (30 mL) was added N-methylmethanamine (2 M, 26.67 mL, 6.10 eq, THF) at 25° C. The resulting mixture was stirred at 70° C. for 8 hours. The combined organic phase was diluted with 60 mL EtOAc and washed with 180 mL aqueous saturated NaHCO₃ (60 mL×3) and 120 mL saturated brine (60 mL×2), dried with anhydrous Na₂SO₄, filtered, and concentrated under reduced pressure to give a residue. The residue was purified by column chromatography (SiO₂, petroleum ether/ethyl acetate=10/1 to 1/1, 3% NH₃·H₂O) to give a compound O1-tert-butyl O2-methyl (2S)-4-[3-(dimethylamino)propoxy]pyrrolidine-1,2-dicarboxylate (2 g, 6.05 mmol, 69.24% yield) as colourless oil.

¹H NMR (400 MHz, CDCl₃), 4.25-4.46 (m, 1H), 3.92-4.13 (m, 1H), 3.68-3.79 (m, 3H), 3.25-3.65 (m, 4H), 2.18-2.35 (m, 9H), 1.98-2.12 (m, 1H), 1.58-1.83 (m, 2H), 1.49-1.51 (m, 9H).

### Step 5

To a solution of O1-tert-butyl O2-methyl (2S)-4-[3-(dimethylamino)propoxy]pyrrolidine-1,2-dicarboxylate (2 g, 6.05 mmol, 1 eq) in THF (5 mL) was added LiOH·H₂O (289.91 mg, 12.11 mmol, 2 eq) in H₂O (5 mL). The mixture was stirred at 25° C. for 8 hours. The crude product was diluted with water (20 mL) and the aqueous phase was freeze-dried to give (2S)-1-tert-butoxycarbonyl-4-[3-(dimethylamino)propoxy]pyrrolidine-2-carboxylic acid (1.5 g, 4.74 mmol, 78.32% yield, Li⁺ salt) as a white solid.

¹H NMR (400 MHz, CDCl₃), 3.88-4.85 (m, 2H), 3.20-3.65 (m, 4H), 2.25-2.52 (m, 10H), 1.65-1.87 (m, 2H), 1.36-1.55 (m, 9H).

### Step 6

A mixture of (2S)-1-tert-butoxycarbonyl-4-[3-(dimethylamino)propoxy]pyrrolidine-2-carboxylic acid (0.85 g, 2.69 mmol, 1 eq), 1-octylnonyl 8-bromooctanoate (1.49 g, 3.22 mmol, 1.2 eq), Cs₂CO₃ (1.93 g, 5.91 mmol, 2.2 eq) in DMF (5 mL) was stirred at 25° C. for 8 hours under N₂ atmosphere. The combined organic phase was diluted with 20 mL EtOAc and washed with 60 mL water (20 mL×3) and 40 mL brine (20 mL×2), dried with anhydrous Na₂SO₄, filtered, and concentrated under reduced pressure to give a residue. The residue was purified by column chromatography (SiO₂, petroleum ether/ethyl acetate/NH₃·H₂O=10/1/0 to 1/1/0.1) to give O1-tert-butyl O2-[8-(1-octylnonoxy)-8-oxo-octyl](2S)-4-[3-(dimethylamino)propoxy]pyrrolidine-1,2-dicarboxylate (0.8 g, 1.15 mmol, 42.72% yield, 100% purity) as colorless oil.

¹H NMR (400 MHz, CDCl₃), 4.81-4.93 (m, 1H), 3.93-4.44 (m, 4H), 3.46-3.73 (m, 4H), 2.20-2.41 (m, 11H), 1.98-2.12 (m, 1H), 1.58-1.79 (m, 7H), 1.39-1.53 (m, 13H), 1.24-1.35 (m, 29H), 0.88 (t, J=6.8 Hz, 6H).

### Step 7

To a solution of O1-tert-butyl O2-[8-(1-octylnonoxy)-8-oxo-octyl](2S)-4-[3-(dimethylamino) propoxy]pyrrolidine-1,2-dicarboxylate (800.00 mg, 1.15 mmol, 1 eq) in DCM (5 mL) was added TFA (23.03 g, 201.93 mmol, 15 mL, 175.94 eq). The mixture was stirred at 25° C. for 2 hours. The reaction mixture was adjusted to pH=7 with aqueous saturated NaHCO₃ and extracted with 60 mL EtOAc (20 mL×3), dried over Na₂SO₄, filtered, and concentrated under reduced pressure to give [8-(1-octylnonoxy)-8-oxo-octyl](2S)-4-[3-(dimethylamino)propoxy]pyrrolidine-2-carboxylate (0.75 g, crude) as yellow oil.

### Step 8

A solution of [8-(1-octylnonoxy)-8-oxo-octyl](2S)-4-[3-(dimethylamino)propoxy]pyrrolidine-2-carboxylate (0.2 g, 335.05 mol, 1 eq), undecyl 6-oxohexanoate (142.95 mg, 502.58 mol, 1.5 eq) in DCM (2 mL) was stirred at 25° C. for 0.5 hour, and then NaBH(OAc)₃ (213.03 mg, 1.01 mmol, 3 eq) was added at 25° C. The resulting mixture was stirred at 25° C. for 7.5 hours. The combined organic phase was diluted with 60 mL EtOAc and washed with 180 mL water (60 mL×3) and 120 mL brine (60 mL×2), dried with anhydrous Na₂SO₄, filtered, and concentrated under reduced pressure to give a residue. The residue was purified by column chromatography (SiO₂, petroleum ether/ethyl acetate/NH₃·H₂O=10/1/1 to 1/1/0.5) to give [8-(1-octylnonoxy)-8-oxo-octyl](2S)-4-[3-(dimethylamino)propoxy]-1-(6-oxo-6-undecoxy-hexyl)pyrrolidine-2-carboxylate (0.12 g, 137.15 mol, 40.93% yield, 98.9% purity) as yellow oil.

¹H NMR (400 MHz, CDCl₃), 4.80-4.95 (m, 1H), 3.92-4.16 (m, 5H), 3.05-3.49 (m, 4H), 2.60-2.78 (m, 1H), 2.24-2.45 (m, 14H), 2.06-2.14 (m, 1H), 1.92-2.03 (m, 1H), 1.73-1.78 (m, 2H), 1.58-1.64 (m, 6H), 1.42-1.51 (m, 6H), 1.11-1.41 (m, 50H), 0.88 (t, J=6.8 Hz, 9H).

LCMS: (M+H⁺): 865.8 @10.120 minutes.

### 8.37. Synthesis of Compound 2436

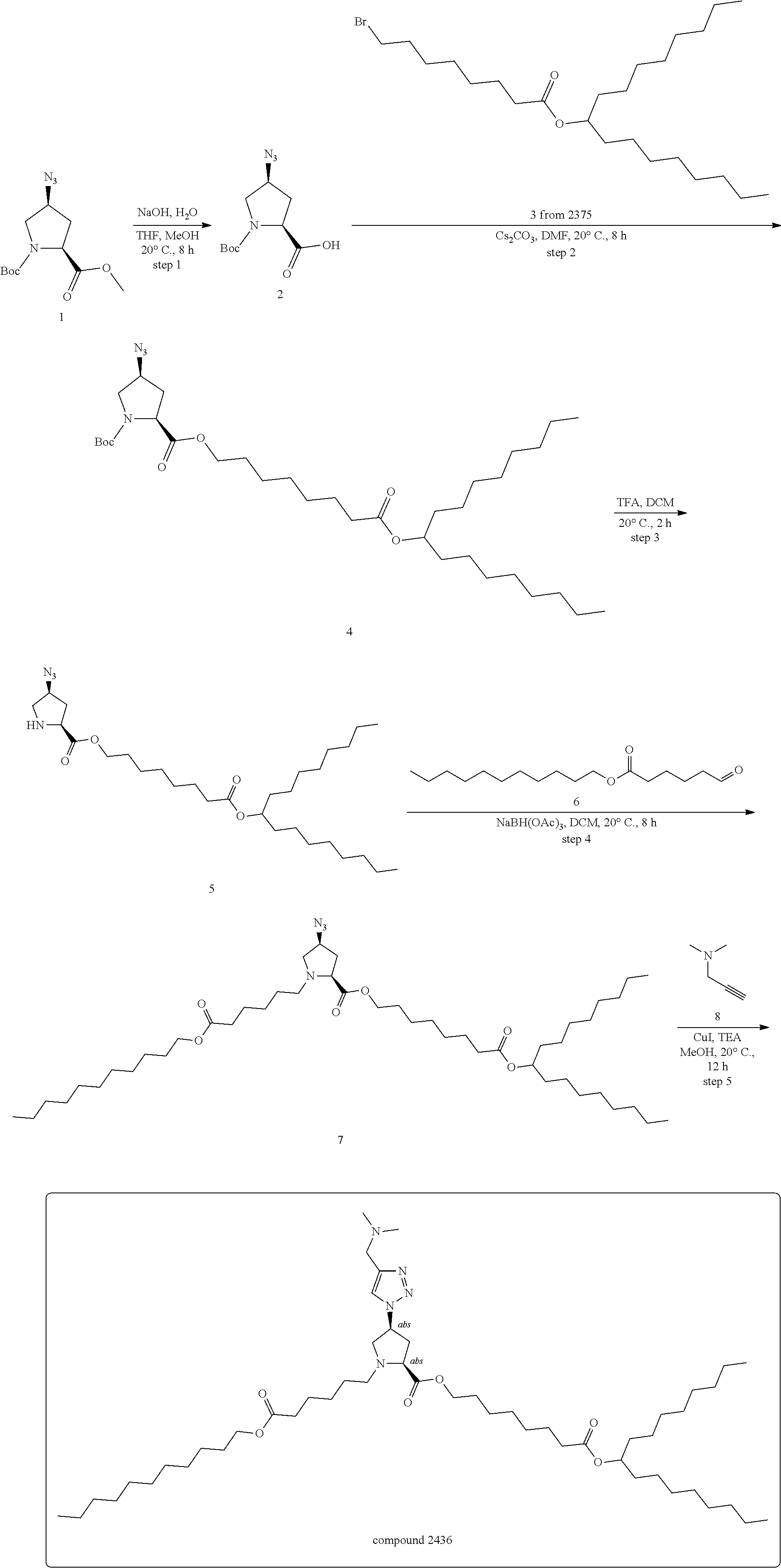

### Step 1

To a solution of O1-tert-butyl O2-methyl (2S,4S)-4-azidopyrrolidine-1,2-dicarboxylate (2.00 g, 7.40 mmol, 1 eq) in THF (20 mL) and MeOH (10 mL) was added a solution of NaOH (1.78 g, 44.40 mmol, 6 eq) in H₂O (7.39 g, 410.48 mmol, 7.39 mL, 55.47 eq). The mixture was stirred at 20° C. for 8 hours. The mixture was adjusted to pH=3 with 1N HCl, and then extracted with 30 mL EtOAc (10 mL×3). The combined organic layers were washed with 30 mL saturated brine (10 mL×3), dried over Na₂SO₄, filtered, and concentrated under reduced pressure to give (2S,4S)-4-azido-1-tert-butoxycarbonyl-pyrrolidine-2-carboxylic acid (1.9 g, crude) as colorless oil, which was used into the next step without further purification.

### Step 2

To a solution of (2S,4S)-4-azido-1-tert-butoxycarbonyl-pyrrolidine-2-carboxylic acid (1.9 g, 7.41 mmol, 1 eq) and 1-octylnonyl 8-bromooctanoate (4.11 g, 8.90 mmol, 1.2 eq) in DMF (100 mL) was added Cs₂CO₃ (5.31 g, 16.31 mmol, 2.2 eq). The mixture was stirred at 20° C. for 8 hours. The mixture was added into H₂O (200 mL), and extracted with EtOAc (200 mL×3). The organic layer was washed with brine (200 mL×2), dried over Na₂SO₄, filtered, and concentrated under reduced pressure. The residue was purified by column chromatography (SiO₂, petroleum ether/ethyl acetate=1/0 to 5/1) to give O1-tert-butyl O2-[8-(1-octylnonoxy)-8-oxo-octyl](2S,4S)-4-azidopyrrolidine-1,2-dicarboxylate (4 g, 6.28 mmol, 84.71% yield) as yellow oil.

¹H NMR (400 MHz, CDCl₃), 4.84-4.89 (m, 1H), 4.30-4.49 (m, 1H), 4.05-4.30 (m, 3H), 3.65-3.85 (m, 1H), 3.40-3.60 (m, 1H), 2.35-2.60 (m, 1H), 2.30 (t, J=7.6 Hz, 2H), 2.10-2.20 (m, 1H), 1.60-1.75 (m, 4H), 1.50-1.60 (m, 4H), 1.47-1.51 (m, 9H), 1.15-1.37 (m, 28H), 0.89 (t, J=6.4 Hz, 6H).

### Step 3

To a solution of O1-tert-butyl O2-[8-(1-octylnonoxy)-8-oxo-octyl](2S,4S)-4-azidopyrrolidine-1,2-dicarboxylate (2 g, 3.14 mmol, 1 eq) in DCM (20 mL) was added TFA (15.35 g, 134.63 mmol, 10 mL, 42.87 eq). The mixture was stirred at 20° C. for 2 hours. The mixture was added into saturated NaHCO₃ (100 mL), and extracted with EtOAc (20 mL×3). The organic layer was washed with brine (20 mL×2), dried over Na₂SO₄, filtered, and concentrated under reduced pressure to give [8-(1-octylnonoxy)-8-oxo-octyl](2S,4S)-4-azidopyrrolidine-2-carboxylate (1.2 g, crude) as yellow oil.

### Step 4

To a solution of [8-(1-octylnonoxy)-8-oxo-octyl](2S,4S)-4-azidopyrrolidine-2-carboxylate (1.2 g, 2.24 mmol, 1 eq) and undecyl 6-oxohexanoate (763.03 mg, 2.68 mmol, 1.2 eq) in DCM (20 mL) was added NaBH(OAc)₃ (1.42 g, 6.71 mmol, 3 eq). The mixture was stirred at 20° C. for 8 hours. The mixture was added into H₂O (20 mL), and extracted with EtOAc (20 mL×3). The organic layer was washed with brine (20 mL×2), dried over Na₂SO₄, filtered, and concentrated under reduced pressure. The residue was purified by column chromatography (SiO₂, petroleum ether/ethyl acetate=1/0 to 5/1) to give [8-(1-octylnonoxy)-8-oxo-octyl](2S,4S)-4-azido-1-(6-oxo-6-undecoxy-hexyl)pyrrolidine-2-carboxylate (1.5 g, 1.86 mmol, 83.33% yield) as yellow oil.

### Step 5

To a solution of [8-(1-octylnonoxy)-8-oxo-octyl](2S,4S)-4-azido-1-(6-oxo-6-undecoxy-hexyl)pyrrolidine-2-carboxylate (500 mg, 620.95 mol, 1 eq), CuI (11.83 mg, 62.09 mol, 0.1 eq) and TEA (6.28 mg, 62.09 mol, 8.64 μL, 0.1 eq) in MeOH (10 mL) was added N,N-dimethylprop-2-yn-1-amine (61.94 mg, 745.14 mol, 79.01 μL, 1.2 eq). The mixture was stirred at 20° C. for 8 hours. The mixture was added into H₂O (20 mL), and extracted with EtOAc (20 mL×3). The organic layer was washed with brine (20 mL×2), dried over Na₂SO₄, filtered, and the filtrate was concentrated under reduced pressure. The residue was purified by column chromatography (SiO₂, petroleum ether/ethyl acetate=10/1 to 0/1, added 5% NH₃·H₂O) to give [8-(1-octylnonoxy)-8-oxo-octyl](2S,4S)-4-[4-[(dimethylamino)methyl]triazol-1-yl]-1-(6-oxo-6-undecoxy-hexyl)pyrrolidine-2-carboxylate (150 mg, 168.85 μmol, 27.19% yield) as yellow oil.

¹H NMR (400 MHz, CDCl₃), 8.12 (s, 1H), 5.30-5.35 (m, 1H), 4.83-4.90 (m, 1H), 4.03-4.20 (m, 4H), 3.61 (s, 2H), 3.23-3.36 (m, 2H), 2.75-2.95 (m, 3H), 2.35-2.45 (m, 1H), 2.25-2.35 (m, 10H), 2.10-2.20 (m, 1H), 1.58-1.64 (m, 8H), 1.45-1.55 (m, 6H), 1.20-1.40 (m, 48H), 0.86-0.91 (m, 9H).

LCMS: (M+H⁺): 888.7 @10.067 minutes.

### 8.38. Synthesis of Compound 2437

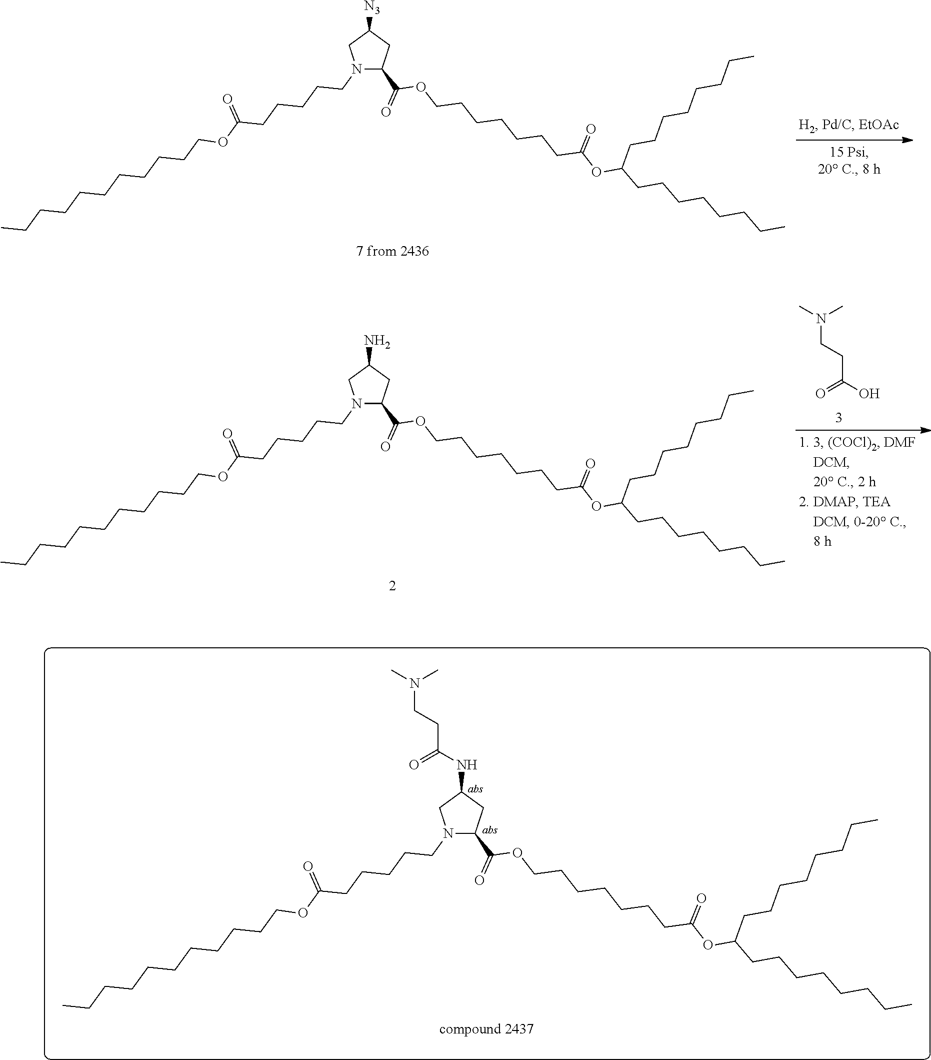

### Step 1

To a solution of Pd/C (660.81 mg, 620.95 μmol, 10% purity, 1 eq) in EtOAc (20 mL) was added [8-(1-octylnonoxy)-8-oxo-octyl](2S,4S)-4-azido-1-(6-oxo-6-undecoxy-hexyl)pyrrolidine-2-carboxylate (500 mg, 620.95 mol, 1 eq), Pd/C (660.81 mg, 620.95 μmol, 10% purity, 1 eq). The mixture was stirred at 20° C. for 8 hours under 15 Psi under H₂. The mixture was filtered and concentrated under reduced pressure to give [8-(1-octylnonoxy)-8-oxo-octyl](2S,4S)-4-amino-1-(6-oxo-6-undecoxy-hexyl)pyrrolidine-2-carboxylate (350 mg, crude) as yellow oil.

### Step 2

To a solution of 3-(dimethylamino)propanoic acid (300 mg, 1.95 mmol, 1 eq, HCl) in DCM (5 mL) was added (COCl)₂ (991.60 mg, 7.81 mmol, 683.86 μL, 4 eq) and DMF (14.27 mg, 195.30 mol, 15.03 μL, 0.1 eq). The mixture was stirred at 20° C. for 2 hours. The mixture was concentrated under reduced pressure to give 3-(dimethylamino)propanoyl chloride (1.6 g, crude, HCl) as a yellow solid. To a solution of [8-(1-octylnonoxy)-8-oxo-octyl](2S,4S)-4-amino-1-(6-oxo-6-undecoxy-hexyl) pyrrolidine-2-carboxylate (350 mg, 449.16 mol, 1 eq) and TEA (227.26 mg, 2.25 mmol, 312.59 μL, 5 eq) in DCM (10 mL) was added DMAP (27.44 mg, 224.58 mol, 0.5 eq) and 3-(dimethylamino)propanoyl chloride (331.15 mg, 1.92 mmol, 4.29 eq, HCl) under N₂ at 0° C., and then the mixture was stirred at 20° C. for 8 hours. The mixture was added into saturated NaHCO₃ (20 mL), and extracted with EtOAc (10 mL×3). The organic layer was washed with brine (10 mL×2), dried over Na₂SO₄, filtered, and the filtrate was concentrated under reduced pressure. The residue was purified by column chromatography (SiO₂, petroleum ether/ethyl acetate=10/1 to 0/1, added 5% NH₃·THF) and prep-HPLC (column: Xselect CSH C18 100×30 mm×5 m; mobile phase: [H₂O(0.04% HCl)-THF:ACN=1:3]; gradient:30%-70% B over 10.0 minutes). The solution was concentrated by lyophilization to give [8-(1-octylnonoxy)-8-oxo-octyl](2S,4S)-4-[3-(dimethylamino) propanoylamino]-1-(6-oxo-6-undecoxy-hexyl)pyrrolidine-2-carboxylate (150 mg, 227.70 mol, 50.69% yield, 96% purity, HCl salt) as yellow oil.

1H NMR (400 MHz, CDCl₃), 11.26-11.52 (m, 2H), 9.67 (s, 1H), 4.83-4.90 (m, 1H), 4.71 (s, 1H), 4.46 (s, 1H), 4.29-4.40 (m, 2H), 4.16 (s, 1H), 4.05 (t, J=6.8 Hz, 2H), 3.35-3.55 (m, 4H), 3.23 (s, 1H), 2.97-3.05 (m, 1H), 2.86-2.95 (m, 6H), 2.75-2.85 (m, 1H), 2.65-2.75 (m, 1H), 2.25-2.35 (m, 4H), 1.90 (s, 1H), 1.58-1.72 (m, 8H), 1.40-1.55 (m, 6H), 1.20-1.38 (m, 48H), 0.89 (t, J=6.4 Hz, 9H). LCMS: (M+H⁺): 878.8 @10.390 minutes.

### 8.39. Synthesis of Compound 2440

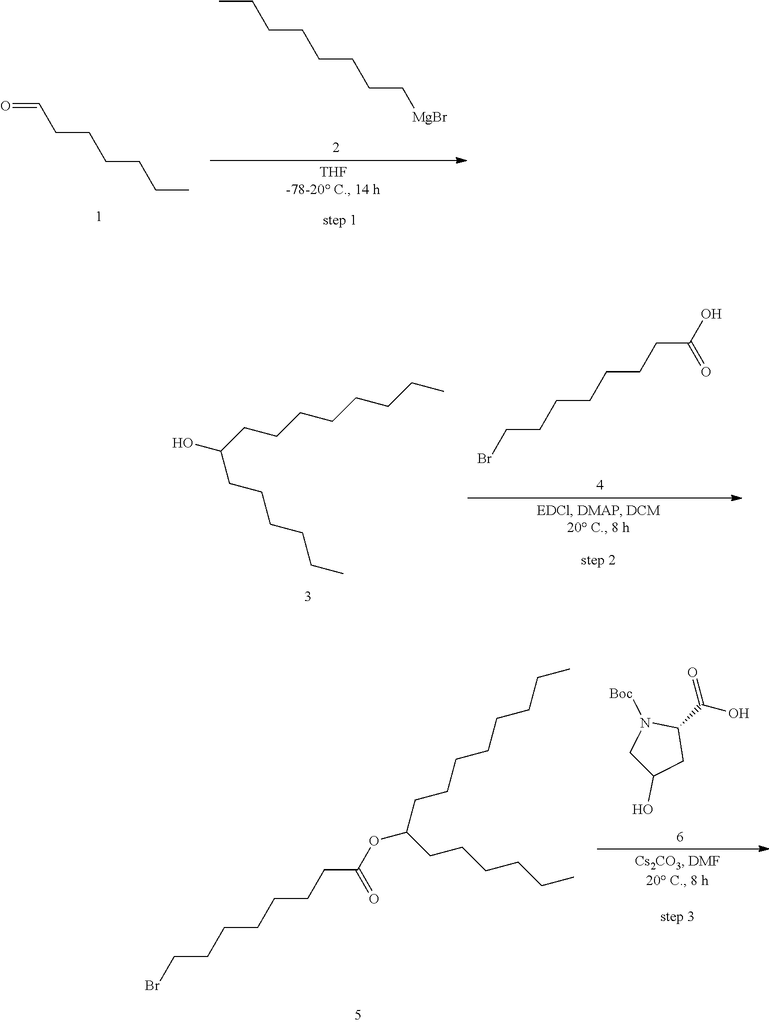

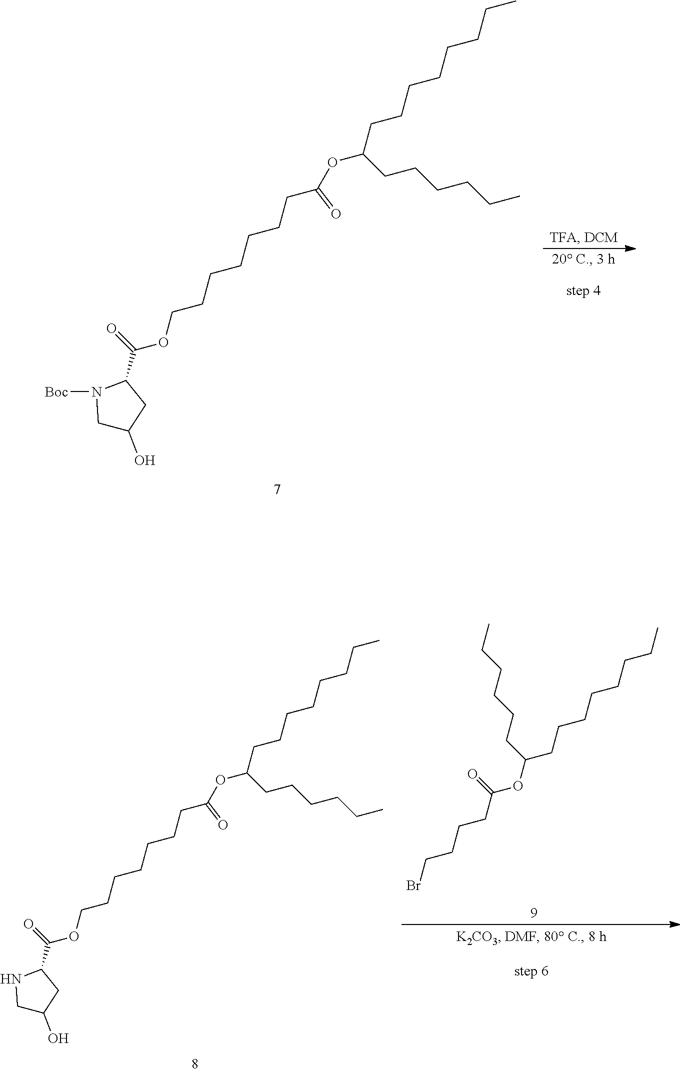 [SMILES: CCCCCCCCC(CCCCCC)OC(=O)CCCCCCCOC(=O)[C@@H]1CC(O)CN1C(=O)OC(C)(C)C ; CCCCCCCCC(CCCCCC)OC(=O)CCCCCCCOC(=O)[C@@H]1CC(O)CN1 ; CCCCCCCCC(CCCCCC)OC(=O)CCCCBr]

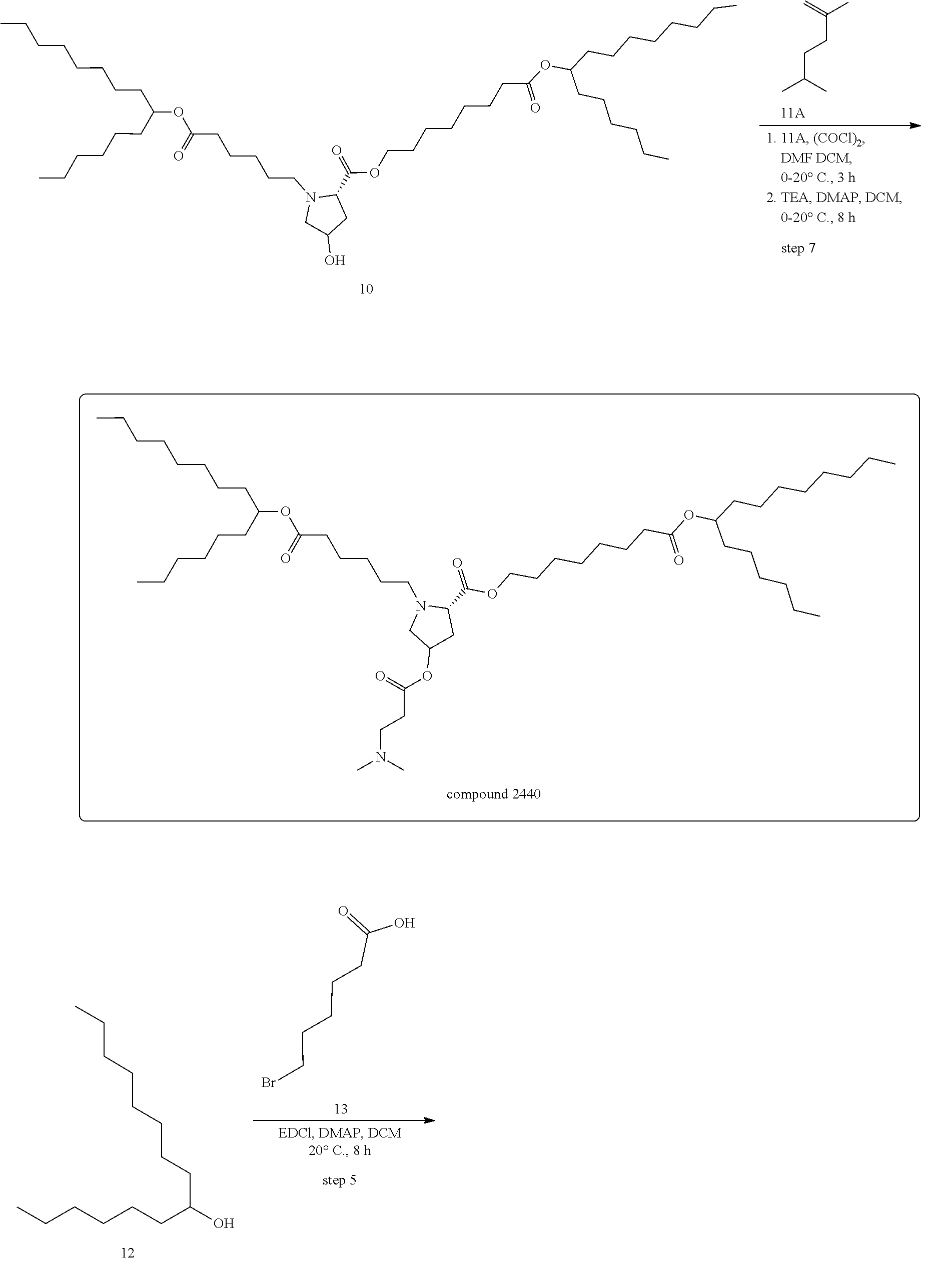

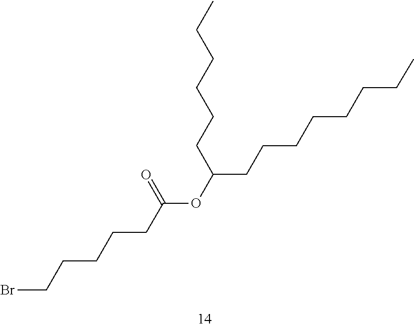 [SMILES: CCCCCCCCC(CCCCCC)OC(=O)CCCCCBr]

### Step 1

To a solution of heptanal (10 g, 87.58 mmol, 12.22 mL, 1 eq) in THF (200 mL) was added bromo(octyl)magnesium (2 M, 48.17 mL, 1.1 eq) at −78° C. Then the mixture was stirred at −78° C. for 2 hours. Then the mixture was stirred at 20° C. for 12 hours. The reaction mixture was diluted with by addition of 1500 mL saturated NH₄C₁, and then extracted with 1500 mL PE (500 mL×3). The combined organic layers were dried over Na₂SO₄, filtered, and concentrated under reduced pressure to give a residue. The residue was purified by column chromatography (SiO₂, petroleum ether/ethyl acetate=1/0) to give a compound pentadecan-7-ol (80 g, 350.24 mmol, 39.99% yield) as a white solid.

### Step 2

To a solution of pentadecan-7-ol (5 g, 21.89 mmol, 1 eq) and 8-bromooctanoic acid (5.13 g, 22.98 mmol, 1.05 eq) in DCM (50 mL) was added EDCI (5.04 g, 26.27 mmol, 1.2 eq) and DMAP (1.34 g, 10.95 mmol, 0.5 eq). The mixture was stirred at 20° C. for 8 hours. The reaction mixture was diluted with 100 mL water and extracted with 150 mL EtOAc (50 mL×3). The combined organic layers were washed with 30 mL brine (10 mL×3), dried over Na₂SO₄, filtered, and concentrated under reduced pressure to give a residue. The residue was purified by column chromatography (SiO₂, petroleum ether/ethyl acetate=1/0) to give 1-hexylnonyl 8-bromooctanoate (7.5 g, 17.30 mmol, 79.03% yield) as colorless oil.

1H NMR (400 MHz, CDCl₃), 4.85-4.91 (m, 1H), 3.41 (t, J=7.2 Hz, 2H), 2.29 (t, J=7.6 Hz, 2H), 1.80-1.95 (m, 2H), 1.62-1.67 (m, 2H), 1.45-1.55 (m, 4H), 1.35-1.39 (m, 2H), 1.27-1.31 (m, 24H), 0.89 (t, J=6.4 Hz, 6H).

### Step 3

To a solution of 1-hexylnonyl 8-bromooctanoate (7.5 g, 17.30 mmol, 1.2 eq) and (2S)-1-tert-butoxycarbonyl-4-hydroxy-pyrrolidine-2-carboxylic acid (3.33 g, 14.42 mmol, 1 eq) in DMF (100 mL) was added Cs₂CO₃ (10.33 g, 31.72 mmol, 2.2 eq) in sequence. Then the mixture was stirred at 20° C. for 8 hours. The reaction mixture diluted with 150 mL H₂O, and then extracted with 150 mL EtOAc (50 mL×3). The combined organic layers were washed with 140 mL brine (70 mL×2), dried over Na₂SO₄, filtered, and concentrated under reduced pressure to give a residue. The residue was purified by column chromatography (SiO₂, petroleum ether/ethyl acetate=1/0 to 3/1) to give O1-tert-butyl O2-[8-(1-hexylnonoxy)-8-oxo-octyl](2S)-4-hydroxypyrrolidine-1,2-dicarboxylate (6 g, 10.28 mmol, 71.28% yield) as yellow oil.

¹H NMR (400 MHz, CDCl₃), 4.85-4.88 (m, 2H), 4.10-4.37 (m, 4H), 3.30-3.68 (m, 2H), 2.26-2.30 (m, 3H), 2.05-2.08 (m, 1H), 1.61-1.67 (m, 2H), 1.42-1.50 (m, 13H), 1.25-1.35 (m, 28H), 0.88 (t, J=6.8 Hz, 6H).

### Step 4

To a solution of O1-tert-butyl O2-[8-(1-hexylnonoxy)-8-oxo-octyl](2S)-4-hydroxypyrrolidine-1,2-dicarboxylate (3.5 g, 5.99 mmol, 1 eq) in DCM (27 mL) was added TFA (13.82 g, 121.16 mmol, 9 mL, 20.21 eq). The mixture was stirred at 20° C. for 3 hours. The reaction mixture was adjusted to pH=7 with aqueous saturated NaHCO₃ and extracted with 150 mL EtOAc (50 mL×3), dried over Na₂SO₄, filtered, and concentrated under reduced pressure to give a residue. The residue was purified by column chromatography (SiO₂, petroleum ether/ethyl acetate=10/1 to 2/1) to give [8-(1-hexylnonoxy)-8-oxo-octyl](2S)-4-hydroxypyrrolidine-2-carboxylate (1.3 g, 2.69 mmol, 43.33% yield) as yellow oil.

### Step 5

A mixture of 6-bromohexanoic acid (4.27 g, 21.89 mmol, 1 eq) in DCM (50 mL) was added EDCI (4.20 g, 21.89 mmol, 1 eq), pentadecan-7-ol (5 g, 21.89 mmol, 1 eq), DMAP (534.86 mg, 4.38 mmol, 0.2 eq) at 20° C. and was degassed and purged with N₂ for 3 times. The mixture was stirred at 20° C. for 8 hours under N₂ atmosphere. The reaction mixture was diluted with 100 mL H₂O and extracted with 150 mL EtOAc (50 mL×3). The combined organic layers were dried over Na₂SO₄, filtered, and concentrated under reduced pressure to give a residue. The residue was purified by column chromatography (SiO₂, petroleum ether/ethyl acetate=1/0 to 40/1) to give 1-hexylnonyl 6-bromohexanoate (8 g, 19.73 mmol, 90.14% yield) as colorless oil.

### Step 6

To a solution of [8-(1-hexylnonoxy)-8-oxo-octyl](2S)-4-hydroxypyrrolidine-2-carboxylate (1.3 g, 2.69 mmol, 1 eq), 1-hexylnonyl 6-bromohexanoate (1.31 g, 3.22 mmol, 1.2 eq) in DMF (20 mL) was added K₂CO₃ (1.11 g, 8.06 mmol, 3 eq). The mixture was stirred at 80° C. for 8 hours. The reaction mixture was diluted with 50 mL H₂O and extracted with 120 mL EtOAc (40 mL×3). The combined organic layers were dried over Na₂SO₄, filtered, and concentrated under reduced pressure to give a residue. The residue was purified by column chromatography (SiO₂, petroleum ether/ethyl acetate=1/0 to 10/1) to give [8-(1-hexylnonoxy)-8-oxo-octyl](2S)-1-[6-(1-hexylnonoxy)-6-oxo-hexyl]-4-hydroxy-pyrrolidine-2-carboxylate (0.4 g, 494.89 mol, 18.41% yield, 100% purity) as colorless oil.

¹H NMR (400 MHz, CDCl₃), 4.86-4.90 (m, 2H), 4.26-4.28 (m, 1H), 4.13 (t, J=6.8 Hz, 2H), 3.05-3.26 (m, 3H), 2.27-2.64 (m, 8H), 1.83-1.92 (m, 1H), 1.61-1.67 (m, 8H), 1.50-1.52 (m, 6H), 1.27-1.45 (m, 50H), 0.89 (t, J=6.8 Hz, 12H).

### Step 7

A mixture of 3-(dimethylamino)propanoic acid (0.6 g, 3.91 mmol, 1 eq, HCl) in DCM (5 mL) was added (COCl)₂ (2.48 g, 19.53 mmol, 1.71 mL, 5 eq), DMF (28.55 mg, 390.61 mol, 30.05 μL, 0.1 eq) at 0° C. The mixture was stirred at 20° C. for 3 hours under N₂ atmosphere. The reaction mixture was concentrated under reduced pressure to give a compound 3-(dimethylamino)propanoyl chloride (0.6 g, crude, HCl) as a white solid. To a solution of [8-(1-hexylnonoxy)-8-oxo-octyl](2S)-1-[6-(1-hexylnonoxy)-6-oxo-hexyl]-4-hydroxy-pyrrolidine-2-carboxylate (0.4 g, 494.89 mol, 1 eq), TEA (250.39 mg, 2.47 mmol, 344.41 μL, 5 eq), DMAP (12.09 mg, 98.98 mol, 0.2 eq) in DCM (10 mL) was added 3-(dimethylamino)propanoyl chloride (425.73 mg, 2.47 mmol, 5 eq, HCl) at 0° C. The mixture was stirred at 20° C. for 8 hours. The reaction mixture was diluted with 20 mL H₂O and extracted with 60 mL EtOAc (20 mL×3). The combined organic layers were dried over Na₂SO₄, filtered, and concentrated under reduced pressure to give a residue. The residue was purified by column chromatography (SiO₂, petroleum ether/ethyl acetate=1/0 to 1/0), prep-HPLC (column: Waters Xbridge BEH C18 100×30 mm×10 μm; mobile phase: [H₂O(0.04% HCl)-THF:ACN=1:3]; gradient:35%-70% B over 10.0 minutes) and prep-HPLC (column: Phenomenex Gemini-NX 80×40 mm×3 μm; mobile phase: [H₂O(0.04% HCl)-THF:ACN=1:3]; gradient:45%-90% B over 10.0 minutes) to give [8-(1-hexylnonoxy)-8-oxo-octyl](2S)-4-[3-(dimethylamino)propanoyloxy]-1-[6-(1-hexylnonoxy)-6-oxo-hexyl]pyrrolidine-2-carboxylate (108 mg, 112.31 mol, 38.15% yield, HCl) as a white solid.

1H NMR (400 MHz, CDCl₃), 11.53-13.46 (m, 2H), 5.37-5.42 (m, 1H), 4.83-4.87 (m, 2H), 4.16-4.51 (m, 4H), 2.31-3.58 (m, 15H), 2.29 (t, J=7.2 Hz, 4H), 1.61-1.87 (m, 14H), 1.26-1.36 (m, 50H), 0.88 (t, J=6.8 Hz, 12H). LCMS: (M+H⁺): 907.7 @10.114 minutes.

### 8.40. Synthesis of Compound 2452

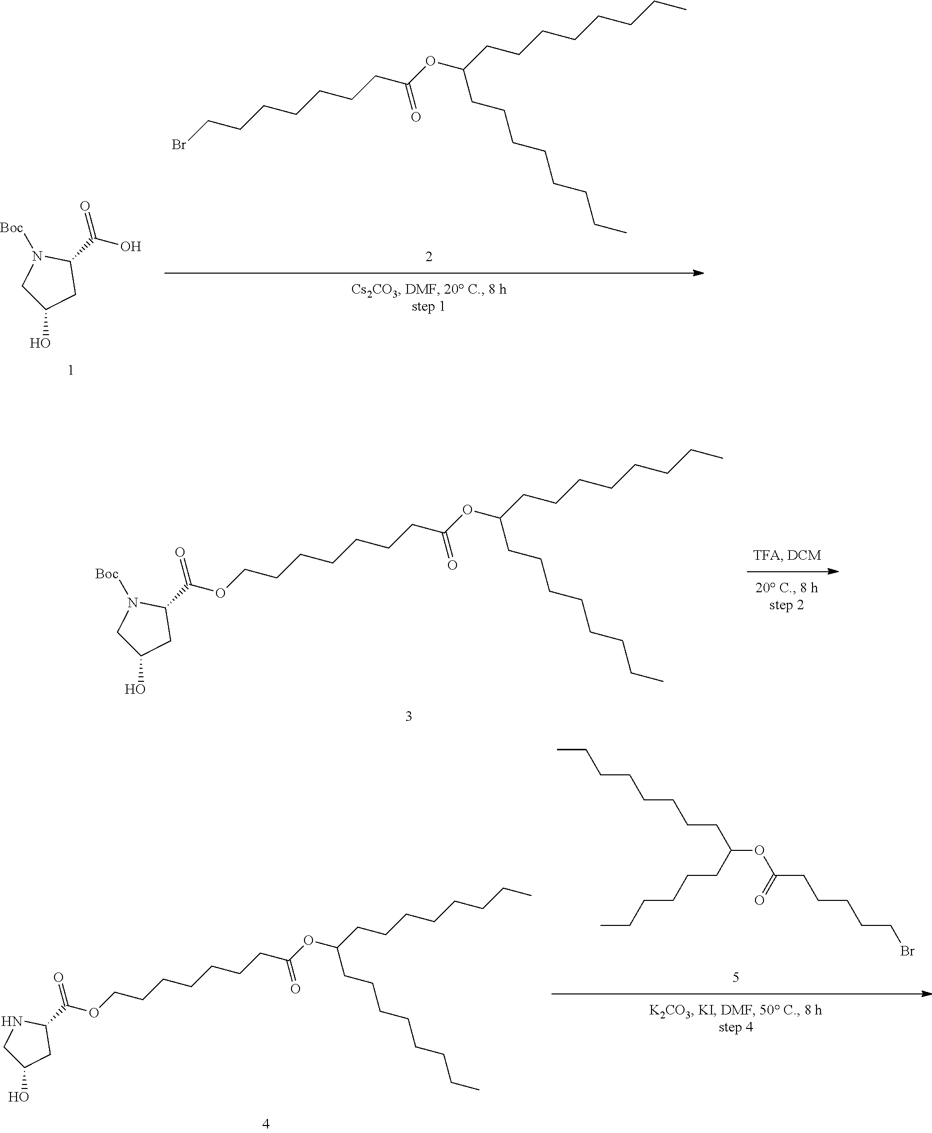

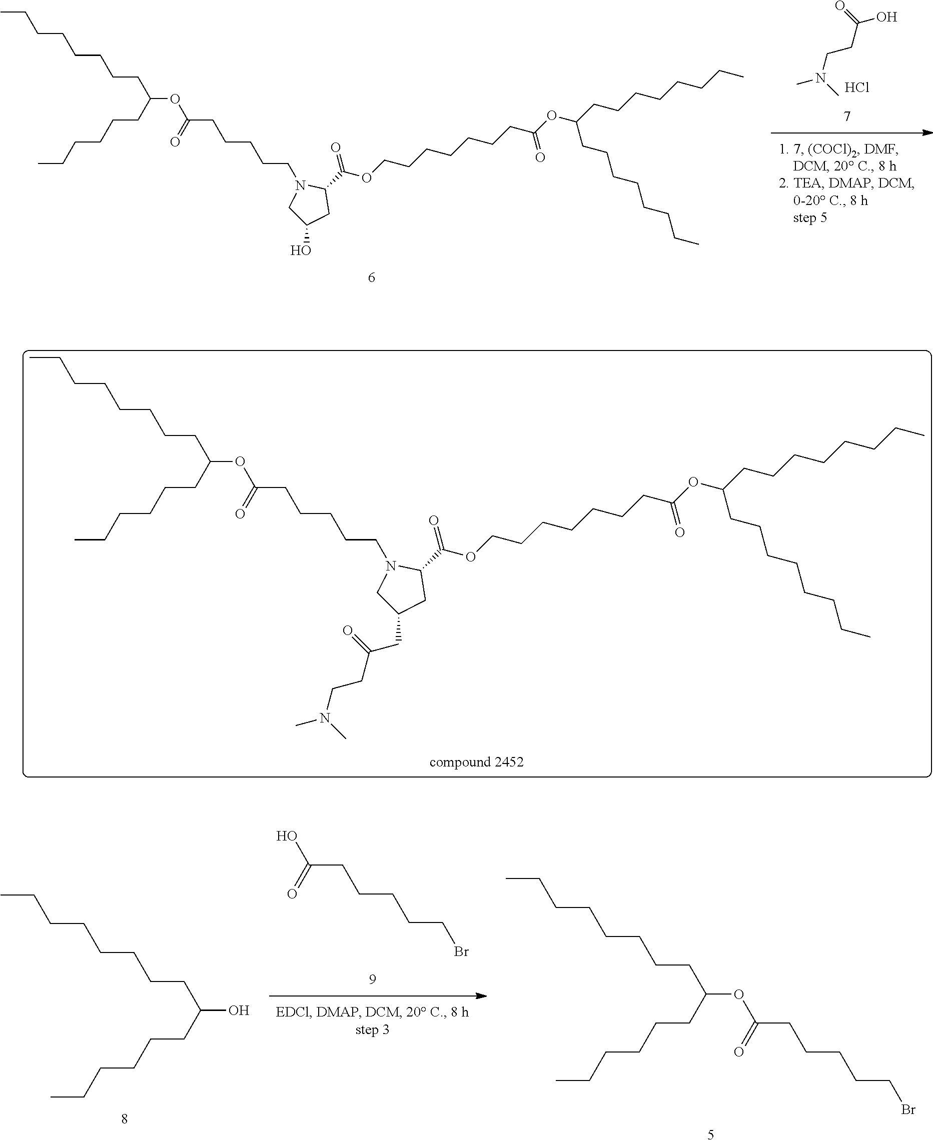

### Step 1

To a solution of (2S,4S)-1-tert-butoxycarbonyl-4-hydroxy-pyrrolidine-2-carboxylic acid (2.2 g, 9.51 mmol, 1 eq) in DMF (30 mL) was added Cs₂CO₃ (4.65 g, 14.27 mmol, 1.5 eq) and 1-octylnonyl 8-bromooctanoate (5.27 g, 11.42 mmol, 1.2 eq). The mixture was stirred at 20° C. for 8 hours. The reaction mixture was quenched by addition of 200 mL H₂O at 0° C., and then extracted with 300 mL EtOAc (100 mL×3). The combined organic layers were dried over Na₂SO₄, filtered, and concentrated under reduced pressure to give a residue. The residue was purified by column chromatography (SiO₂, petroleum ether/ethyl acetate=20/1 to 0/1) to give O1-tert-butyl O2-[8-(1-octylnonoxy)-8-oxo-octyl](2S,4S)-4-hydroxypyrrolidine-1,2-dicarboxylate (20 g, 32.69 mmol, 85.87% yield) as colorless oil.

¹H NMR (400 MHz, CDCl₃), 4.84-4.89 (m, 1H), 4.14-4.36 (m, 4H), 3.53-3.68 (m, 2H), 2.28-2.35 (m, 3H), 2.06-2.10 (m, 1H), 1.51-1.66 (m, 4H), 1.46-1.49 (m, 14H), 1.25-1.34 (m, 32H), 0.88 (t, J=6.4H, 6H).

### Step 2

To a solution of O1-tert-butyl O2-[8-(1-octylnonoxy)-8-oxo-octyl](2S,4S)-4-hydroxypyrrolidine-1,2-dicarboxylate (4 g, 6.54 mmol, 1 eq) in DCM (30 mL) was added TFA (15 mL). The mixture was stirred at 20° C. for 8 hours. The mixture was concentrated under reduced pressure, then adjusted to pH=8 with saturated NaHCO₃, and extracted with 200 mL EtOAc (40 mL×3). The combined organic layers were dried over Na₂SO₄, filtered, and concentrated under reduced pressure to give a residue. The residue was purified by column chromatography (SiO₂, petroleum ether/ethyl acetate=20/1 to 0/1) to give [8-(1-octylnonoxy)-8-oxo-octyl](2S,4S)-4-hydroxypyrrolidine-2-carboxylate (16 g, 31.26 mmol, 95.64% yield) as colorless oil.

### Step 3

To a solution of 6-bromohexanoic acid (6.75 g, 34.59 mmol, 1.58 eq) in DCM (50 mL) was added EDCI (6.29 g, 32.84 mmol, 1.5 eq), DMAP (802.28 mg, 6.57 mmol, 0.3 eq) and pentadecan-7-ol (5 g, 21.89 mmol, 1 eq). The mixture was stirred at 20° C. for 8 hours. The reaction mixture was quenched by addition of 50 mL H₂O at 0° C., and then extracted with 90 mL EtOAc (30 mL×3). The combined organic layers were dried over Na₂SO₄, filtered, and concentrated under reduced pressure to give 1-hexylnonyl 6-bromohexanoate (8.88 g, crude) as colorless oil.

### Step 4

To a solution of [8-(1-octylnonoxy)-8-oxo-octyl](2S,4S)-4-hydroxypyrrolidine-2-carboxylate (3.6 g, 7.03 mmol, 1 eq) in DMF (90 mL) was added K₂CO₃ (2.92 g, 21.10 mmol, 3 eq) and KI (1.17 g, 7.03 mmol, 1 eq) and 1-hexylnonyl 6-bromohexanoate (8.56 g, 21.10 mmol, 3 eq). The mixture was stirred at 50° C. for 8 hours. The reaction mixture was quenched by addition of 50 mL H₂O at 0° C., and then extracted with 150 mL EtOAc (50 mL×3). The combined organic layers were dried over Na₂SO₄, filtered, and concentrated under reduced pressure to give a residue. The residue was purified by column chromatography (SiO₂, petroleum ether/ethyl acetate=50/1 to 0/1) to give [8-(1-octylnonoxy)-8-oxo-octyl](2S,4S)-1-[6-(1-hexylnonoxy)-6-oxo-hexyl]-4-hydroxy-pyrrolidine-2-carboxylate (3 g, 3.57 mmol, 50.77% yield, 99.5% purity) as yellow oil.

¹H NMR (400 MHz, CDCl₃), 4.83-4.89 (m, 2H), 4.11-4.26 (m, 3H), 3.05-3.67 (m, 3H), 2.60-2.63 (m, 3H), 2.26-2.30 (m, 5H), 1.90-1.93 (m, 1H), 1.61-1.66 (m, 6H), 1.50-1.51 (m, 9H), 1.26-1.35 (m, 52H), 0.88 (t, J=6.4H, 12H).

### Step 5

To a solution of 3-(dimethylamino)propanoic acid (480 mg, 3.12 mmol, 1 eq, HCl) in DCM (5 mL) was added DMF (11.42 mg, 156.24 mol, 12.02 μL, 0.05 eq) and oxalyl dichloride (475.95 mg, 3.75 mmol, 328.24 μL, 1.2 eq). The mixture was stirred at 20° C. for 8 hours. The mixture was concentrated under reduced pressure to give 3-(dimethylamino)propanoyl chloride (537.6 mg, crude, HCl) as yellow oil. The crude oil residue was dissolved with DCM (10 mL), then added into a solution of [8-(1-octylnonoxy)-8-oxo-octyl](2S,4S)-1-[6-(1-hexylnonoxy)-6-oxo-hexyl]-4-hydroxy-pyrrolidine-2-carboxylate (500 mg, 597.86 mol, 1 eq), TEA (604.97 mg, 5.98 mmol, 832.15 μL, 10 eq) and DMAP (36.52 mg, 298.93 mol, 0.5 eq) in DCM (5 mL) at 0° C. The mixture was stirred at 20° C. for 8 hours. The reaction mixture was quenched by addition of 10 mL H₂O at 0° C., and then extracted with 30 mL EtOAc (10 mL×3). The combined organic layers were dried over Na₂SO₄, filtered, and concentrated under reduced pressure to give a residue. The residue was purified by column chromatography (SiO₂, petroleum ether/ethyl acetate=1/0 to 1/1, added 0.1% NH₃—H₂O) to give [8-(1-octylnonoxy)-8-oxo-octyl](2S,4S)-4-[3-(dimethylamino) propanoyloxy]-1-[6-(1-hexylnonoxy)-6-oxo-hexyl]pyrrolidine-2-carboxylate (280 mg, 299.32 mol, 50.07% yield) as yellow oil.

¹H NMR (400 MHz, CDCl₃), 12.36-13.34 (m, 2H), 5.36 (brs, 1H), 4.82-4.89 (m, 2H), 4.29-4.45 (m, 4H), 2.83-3.56 (m, 16H), 2.29 (t, J=7.6 Hz, 4H), 1.61-1.72 (m, 6H), 1.50 (brs, 8H), 1.26-1.36 (m, 54H), 0.88 (t, J=6.8H, 12H).

### Example 9. Preparation of Lipid Nanoparticle Compositions

### Exemplary Lipid Nanoparticle Compositions

Exemplary lipid nanoparticle compositions were prepared to result in an ionizable lipid: structural lipid:sterol:PEG-lipid at a molar ratio shown in the below charts.

Molar ratios of the lipid components of each lipid nanoparticle composition are summarized below.

| | | Molar ratio | | | |
|---|---|---|---|---|---|
| Ionizable | | Ionizable | Structural | Plant | DMPE- |
| Lipid No. | mRNA | component | DSPC | Cholesterol | PEG2k |
| 2230 | FLUC/EPO 1:1 | 50 | 10 | 38.5 | 1.5 |
| 2231 | FLUC/EPO 1:1 | 50 | 10 | 38.5 | 1.5 |
| 2260 | FLUC/EPO 1:1 | 50 | 10 | 38.5 | 1.5 |
| 2270 | FLUC/EPO 1:1 | 50 | 10 | 38.5 | 1.5 |
| 2290 | FLUC/EPO 1:1 | 50 | 10 | 38.5 | 1.5 |
| 2291 | FLUC/EPO 1:1 | 50 | 10 | 38.5 | 1.5 |
| 2293 | FLUC/EPO 1:1 | 50 | 10 | 38.5 | 1.5 |
| 2298 | FLUC/EPO 1:1 | 50 | 10 | 38.5 | 1.5 |
| 2306 | FLUC/EPO 1:1 | 50 | 10 | 38.5 | 1.5 |
| 2307 | FLUC/EPO 1:1 | 50 | 10 | 38.5 | 1.5 |
| 2308 | FLUC/EPO 1:1 | 50 | 10 | 38.5 | 1.5 |
| 2309 | FLUC/EPO 1:1 | 50 | 10 | 38.5 | 1.5 |
| 2310 | FLUC/EPO 1:1 | 50 | 10 | 38.5 | 1.5 |
| 2336 | FLUC/EPO 1:1 | 50 | 10 | 38.5 | 1.5 |
| 2337 | FLUC/EPO 1:1 | 50 | 10 | 38.5 | 1.5 |
| 2338 | FLUC/EPO 1:1 | 50 | 10 | 38.5 | 1.5 |
| 2339 | FLUC/EPO 1:1 | 50 | 10 | 38.5 | 1.5 |
| 2340 | FLUC/EPO 1:1 | 50 | 10 | 38.5 | 1.5 |
| 2341 | FLUC/EPO 1:1 | 50 | 10 | 38.5 | 1.5 |
| 2342 | FLUC/EPO 1:1 | 50 | 10 | 38.5 | 1.5 |
| 2343 | FLUC/EPO 1:1 | 50 | 10 | 38.5 | 1.5 |
| 2344 | FLUC/EPO 1:1 | 50 | 10 | 38.5 | 1.5 |
| 2345 | FLUC/EPO 1:1 | 50 | 10 | 38.5 | 1.5 |
| 2348 | FLUC/EPO 1:1 | 50 | 10 | 38.5 | 1.5 |
| 2349 | FLUC/EPO 1:1 | 50 | 10 | 38.5 | 1.5 |
| 2352 | FLUC/EPO 1:1 | 50 | 10 | 38.5 | 1.5 |
| 2371 | FLUC/EPO 1:1 | 50 | 10 | 38.5 | 1.5 |
| 2372 | FLUC/EPO 1:1 | 50 | 10 | 38.5 | 1.5 |
| 2373 | FLUC/EPO 1:1 | 50 | 10 | 38.5 | 1.5 |
| 2375 | FLUC/EPO 1:1 | 50 | 10 | 38.5 | 1.5 |
| 2376 | FLUC/EPO 1:1 | 50 | 10 | 38.5 | 1.5 |
| 2377 | FLUC/EPO 1:1 | 50 | 10 | 38.5 | 1.5 |
| 2436 | FLUC/EPO 1:1 | 50 | 10 | 38.5 | 1.5 |
| 2452 | FLUC/EPO 1:1 | 50 | 10 | 38.5 | 1.5 |

To prepare the exemplary lipid nanoparticle compositions, the lipid components according to the above chart were solubilized in ethanol, mixed at the above-indicated molar ratios, and diluted in ethanol (organic phase) to obtain total lipid concentration of 5.5 mM.

### Lipid Nanoparticle Compositions Encapsulating mRNA

An mRNA solution (aqueous phase, fluc:EPO mRNA), according to the above chart for each LNP composition, was prepared with RNAse-free water and 100 mM citrate buffer pH 3 for a final concentration of 50 mM citrate buffer and 0.167 mg/mL mRNA concentration (1:1 Fluc:EPO). The formulations were maintained at an ionizable lipid to mRNA at an ionizable lipid nitrogen:mRNA phosphate (N:P) ratio of 6:1.

For each LNP composition, the lipid mix and mRNA solution were mixed at a 1:3 ratio by volume, respectively, on a NanoAssemblr Ignite (Precision Nanosystems) at a total flow rate of 9 mL/min. The resulting compositions were then loaded into Slide-A-Lyzer G2 dialysis cassettes (10k MWCO) and dialyzed in 200 times sample volume of 1×PBS for 2 hours at room temperature with gentle stirring. The PBS was refreshed, and the compositions were further dialyzed for at least 14 hours at 4° C. with gentle stirring. The dialyzed compositions were then collected and concentrated by centrifugation at 2000×g using Amicon Ultra centrifugation filters (100k MWCO). The concentrated particles were characterized for size, polydispersity, and particle concentration using Zetasizer Ultra (Malvern Panalytical) and for mRNA encapsulation efficiency using Quant-iT RiboGreen RNA Assay Kit (ThermoFisher Scientific).

For pKa measurement, a TNS assay was conducted according to those described in Sabnis et al., Molecular Therapy, 26(6):1509-19), which is incorporated herein by reference in its entirety. Briefly, 20 buffers (10 mM sodium phosphate, 10 mM sodium borate, 10 mM sodium citrate, and 150 mM sodium chloride, in distilled Water) of unique pH values ranging form 3.0-12.0 were prepared using 1M sodium hydroxide and IM hydrochloric acid. 3.25 μL of a LNP composition (0.04 mg/mL mRNA, in PBS) was incubated with 2 μL of TNS reagent (0.3 mM, in DMSO) and 90 μL of buffer for each pH value (described above) in a 96-well black-walled plate. Each pH condition was performed in triplicate wells. The TNS fluorescence was measured using a Biotek Cytation Plate reader at excitation/emission wavelengths of 321/445 nm. The fluorescence values were then plotted and fit using a 4-parameter sigmoid curve. From the fit, the pH value yielding the half-maximal fluorescence was calculated and reported as the apparent LNP pKa value.

The particle characterization data for each exemplary lipid nanoparticle composition, labeled by the same ionizable lipid number based on which it was prepared, are shown in the table below.

| LNP No. | mRNA | Size (nm) | PDI | % EE | pKa (TNS) |
|---|---|---|---|---|---|
| 2230 | FLUC/EPO 1:1 | 87.6 | 0.10 | 92.4 | 4.84 |
| 2231 | FLUC/EPO 1:1 | 91.6 | 0.05 | 93.9 | 5.03 |
| 2260 | FLUC/EPO 1:1 | 62.6 | 0.14 | 90 | 4.72 |
| 2270 | FLUC/EPO 1:1 | 68.4 | 0.02 | 88.7 | 4.85 |
| 2290 | FLUC/EPO 1:1 | 117.5 | 0.06 | 94.6 | 5.76 |
| 2291 | FLUC/EPO 1:1 | 93.2 | 0.10 | 95 | 4.65 |
| 2293 | FLUC/EPO 1:1 | 92.3 | 0.05 | 86.5 | 6.02 |
| 2298 | FLUC/EPO 1:1 | 120.7 | 0.09 | 90 | 5.14 |
| 2306 | FLUC/EPO 1:1 | 124.5 | 0.08 | 94.8 | 5.20 |
| 2307 | FLUC/EPO 1:1 | 173.0 | 0.09 | 95.9 | |
| 2308 | FLUC/EPO 1:1 | 98.3 | 0.06 | 96.5 | 5.70 |
| 2309 | FLUC/EPO 1:1 | 128.1 | 0.10 | 96.7 | |
| 2310 | FLUC/EPO 1:1 | 138.5 | 0.13 | 96.1 | |
| 2336 | FLUC/EPO 1:1 | 92.1 | 0.05 | 93.4 | 5.67 |
| 2337 | FLUC/EPO 1:1 | 123.8 | 0.06 | 90.1 | 5.78 |
| 2338 | FLUC/EPO 1:1 | 120.8 | 0.10 | 89.3 | 5.91 |
| 2339 | FLUC/EPO 1:1 | 79.2 | 0.07 | 95.8 | 4.68 |
| 2340 | FLUC/EPO 1:1 | 123.2 | 0.09 | 89.3 | 5.93 |
| 2341 | FLUC/EPO 1:1 | 104.2 | 0.10 | 89.1 | 5.58 |
| 2342 | FLUC/EPO 1:1 | 113.4 | 0.05 | 89 | 5.81 |
| 2343 | FLUC/EPO 1:1 | 90.8 | 0.07 | 93.5 | 4.93 |
| 2344 | FLUC/EPO 1:1 | 88.1 | 0.03 | 91.5 | 5.54 |
| 2345 | FLUC/EPO 1:1 | 91.8 | 0.03 | 90.1 | |
| 2348 | FLUC/EPO 1:1 | 105.4 | 0.08 | 93.6 | 5.59 |
| 2349 | FLUC/EPO 1:1 | 90.4 | 0.13 | 96.8 | |
| 2352 | FLUC/EPO 1:1 | 151.7 | 0.32 | 32.8 | |
| 2371 | FLUC/EPO 1:1 | 107.0 | 0.05 | 94.7 | 6.17 |
| 2372 | FLUC/EPO 1:1 | 95.8 | 0.06 | 95.6 | 5.98 |
| 2373 | FLUC/EPO 1:1 | 84.8 | 0.34 | 97.9 | 7.28 |
| 2375 | FLUC/EPO 1:1 | 127.6 | 0.12 | 96.7 | 6.77 |
| 2376 | FLUC/EPO 1:1 | 104.1 | 0.04 | 97.4 | 7.66 |
| 2377 | FLUC/EPO 1:1 | 101.2 | 0.33 | 96.5 | 7.78 |
| 2436 | FLUC/EPO 1:1 | 134.0 | 0.15 | 93.4 | 6.60 |
| 2452 | FLUC/EPO 1:1 | 109.2 | 0.11 | 95.2 | 5.91 |

### Example 10. In-Vivo Bioluminescent Imaging

The exemplary lipid nanoparticle compositions prepared according to Example 9, with encapsulating an mRNA according to the table shown above in Example 9, were used in this example.

### Bioluminescence Screening

8-9 week old female Balb/c mice were utilized for bioluminescence-based ionizable lipid screening efforts. Mice were obtained from Jackson Laboratories (JAX Stock: 000651) and allowed to acclimate for one week prior to manipulations. Animals were placed under a heat lamp for a few minutes before introducing them to a restraining chamber. The tail was wiped with alcohol pads (Fisher Scientific) and, for each LNP composition described above, 100 μL of a lipid nanoparticle composition described above containing 10 μg total mRNA (5 μg Fluc+5 μg EPO) was injected intravenously using a 29G insulin syringe (Covidien). 4-6 hours post-dose, animals were injected with 200 μL of 15 mg/mL D-Luciferin (GoldBio), and placed in set nose cones inside the IVIS Lumina LT imager (PerkinElmer). LivingImage software was utilized for imaging. Whole body bio-luminescence was captured at auto-exposure after which animals are removed from the IVIS and placed into a CO₂ chamber for euthanasia. Cardiac puncture was performed on each animal after placing it in dorsal recumbency, and blood collection was performed using a 25G insulin syringe (BD). Once all blood samples were collected, tubes are spun at 2000 G for 10 minutes using a tabletop centrifuge and plasma was aliquoted into individual Eppendorf tubes (Fisher Scientific) and stored at −80° C. for subsequent EPO quantification. EPO levels in plasma were determined using EPO MSD kit (Meso Scale Diagnostics). The hEPO MSD measurement protocol was the same as those described in Section hEPO MSD Measurement in Example 7.

The average radiance levels determined by the in-vivo bioluminescent imaging for each lipid nanoparticle compositions are shown in the table below.

| | | Bioluminescence (IV) | | | | | |
|---|---|---|---|---|---|---|---|
| LNP | | Whole | | | | | Spleen:Liver |
| No. | mRNA dose | Body | Liver | Spleen | Lung | hEPO | Ratio |
| 2230 | 5 μg FLUC + | 4.8E+06 | 6.6E+05 | 1.6E+06 | 1.0E+04 | 1.2E+05 | 2.409 |
| | 5 μg EPO | | | | | | |
| 2231 | 5 μg FLUC + | 2.4E+06 | 4.2E+05 | 3.8E+06 | 1.8E+04 | 4.7E+04 | 12.767 |
| | 5 μg EPO | | | | | | |
| 2260 | 5 μg FLUC + | 4.2E+04 | 6.1E+03 | 9.7E+03 | 1.2E+03 | 2.2E+02 | 2.501 |
| | 5 μg EPO | | | | | | |
| 2270 | 5 μg FLUC + | 1.4E+04 | 3.0E+03 | 6.4E+03 | 1.1E+03 | 9.1E+01 | 2.260 |
| | 5 μg EPO | | | | | | |
| 2290 | 5 μg FLUC + | 1.5E+07 | 1.8E+06 | 4.9E+06 | 2.8E+04 | 1.5E+05 | 2.707 |
| | 5 μg EPO | | | | | | |
| 2291 | 5 μg FLUC + | 1.0E+05 | 1.2E+04 | 3.2E+05 | 1.7E+03 | 1.6E+03 | 27.074 |
| | 5 μg EPO | | | | | | |
| 2293 | 5 μg FLUC + | 1.9E+04 | 4.0E+03 | 2.9E+05 | 3.3E+03 | 5.8E+02 | 72.893 |
| | 5 μg EPO | | | | | | |
| 2298 | 5 μg FLUC + | 2.4E+07 | 3.0E+06 | 8.6E+06 | 3.1E+04 | 1.4E+05 | 3.448 |
| | 5 μg EPO | | | | | | |
| 2306 | 5 μg FLUC + | 2.5E+07 | 2.6E+06 | 6.4E+06 | 2.5E+04 | 2.2E+05 | 3.685 |
| | 5 μg EPO | | | | | | |
| 2307 | 5 μg FLUC + | 7.7E+04 | 1.1E+04 | 7.5E+04 | 1.8E+03 | 4.9E+03 | 6.690 |
| | 5 μg EPO | | | | | | |
| 2308 | 5 μg FLUC + | 8.5E+05 | 6.6E+04 | 7.8E+06 | 1.2E+04 | 1.1E+04 | 112.706 |
| | 5 μg EPO | | | | | | |
| 2309 | 5 μg FLUC + | 7.4E+06 | 8.8E+05 | 5.1E+06 | 4.4E+04 | 8.1E+04 | 5.742 |
| | 5 μg EPO | | | | | | |
| 2310 | 5 μg FLUC + | 1.3E+07 | 8.9E+05 | 4.8E+06 | 1.1E+05 | 1.09E+05 | 5.580 |
| | 5 μg EPO | | | | | | |
| 2336 | 5 μg FLUC + | 1.1E+05 | 2.1E+04 | 9.6E+04 | 1.4E+03 | 1.2E+03 | 5.843 |
| | 5 μg EPO | | | | | | |
| 2337 | 5 μg FLUC + | 5.8E+06 | 1.6E+06 | 8.4E+06 | 1.5E+05 | 1.3E+05 | 5.281 |
| | 5 μg EPO | | | | | | |
| 2338 | 5 μg FLUC + | 7.8E+06 | 8.4E+05 | 3.5E+06 | 1.3E+04 | 1.7E+05 | 4.171 |
| | 5 μg EPO | | | | | | |
| 2339 | 5 μg FLUC + | 2.2E+04 | 4.0E+03 | 3.1E+05 | 7.8E+03 | 7.6E+03 | 79.023 |
| | 5 μg EPO | | | | | | |
| 2340 | 5 μg FLUC + | 3.2E+07 | 3.6E+06 | 7.5E+06 | 4.9E+04 | 3.1E+05 | 1.993 |
| | 5 μg EPO | | | | | | |
| 2341 | 5 μg FLUC + | 1.3E+07 | 2.2E+06 | 1.2E+06 | 8.9E+03 | 2.7E+05 | 0.559 |
| | 5 μg EPO | | | | | | |
| 2342 | 5 μg FLUC + | 1.2E+07 | 2.3E+06 | 6.9E+06 | 4.1E+04 | 8.9E+04 | 3.514 |
| | 5 μg EPO | | | | | | |
| 2343 | 5 μg FLUC + | 5.0E+07 | 8.4E+06 | 9.4E+05 | 7.0E+03 | 7.5E+05 | 0.113 |
| | 5 μg EPO | | | | | | |
| 2344 | 5 μg FLUC + | 2.0E+08 | 3.7E+07 | 4.2E+06 | 3.3E+04 | 1.4E+06 | 0.123 |
| | 5 μg EPO | | | | | | |
| 2345 | 5 μg FLUC + | 8.4E+07 | 1.7E+07 | 4.2E+06 | 7.1E+03 | 5.3E+06 | 0.239 |
| | 5 μg EPO | | | | | | |
| 2348 | 5 μg FLUC + | 2.5E+05 | 3.8E+04 | 4.6E+05 | 4.4E+03 | 7.3E+03 | 12.192 |
| | 5 μg EPO | | | | | | |
| 2349 | 5 μg FLUC + | 5.3E+04 | 8.0E+03 | 2.6E+04 | 7.8E+02 | 3.8E+02 | 4.520 |
| | 5 μg EPO | | | | | | |
| 2352 | 5 μg FLUC + | 2.2E+04 | 4.1E+03 | 2.3E+03 | 7.8E+02 | 4.1E+02 | 0.599 |
| | 5 μg EPO | | | | | | |
| 2371 | 5 μg FLUC + | 1.6E+07 | 3.2E+06 | 5.3E+06 | 1.9E+04 | 2.5E+05 | 1.647 |
| | 5 μg EPO | | | | | | |
| 2372 | 5 μg FLUC + | 5.9E+04 | 1.4E+04 | 6.2E+04 | 2.5E+02 | 4.3E+04 | 4.623 |
| | 5 μg EPO | | | | | | |
| 2373 | 5 μg FLUC + | 7.9E+03 | 1.2E+03 | 7.2E+03 | 1.1E+03 | 1.9E+03 | 6.493 |
| | 5 μg EPO | | | | | | |
| 2375 | 5 μg FLUC + | 1.2E+04 | 1.7E+03 | 2.1E+04 | 1.1E+03 | 1.2E+02 | 12.568 |
| | 5 μg EPO | | | | | | |
| 2376 | 5 μg FLUC + | 3.0E+03 | 4.6E+02 | 5.8E+04 | 1.4E+03 | 6.9E+02 | 112.877 |
| | 5 μg EPO | | | | | | |
| 2377 | 5 μg FLUC + | 2.3E+04 | 2.7E+03 | 2.6E+05 | 3.9E+03 | 3.1E+02 | 109.114 |
| | 5 μg EPO | | | | | | |
| 2436 | 5 μg FLUC + | 7.14E+05 | 1.05E+05 | 6.25E+05 | 2.62E+03 | 1.3E+04 | 5.93 |
| | 5 μg EPO | | | | | | |
| 2452 | 5 μg FLUC + | 1.62E+06 | 1.59E+05 | 2.59E+05 | 2.61E+03 | 1.73E+05 | 1.855 |
| | 5 μg EPO | | | | | | |

As can be seen, the lipid nanoparticle compositions containing the novel ionizable lipid compounds demonstrate selective delivery of the therapeutic cargos outside the liver and, due to the lower lipid levels in the liver, lower liver toxicity is expected.

In particular, the spleen:liver ratio of average radiance was determined for all the exemplary lipid nanoparticle compositions. As discussed in Example 7, the comparative lipid nanoparticle compositions (LNP C12-200, LNP MC3) had a very low spleen to liver ratio (<<0.1), whereas all the exemplary lipid nanoparticle compositions had exhibited a significantly higher spleen to liver ratio than that of the comparative lipid nanoparticle compositions (LNP C12-200, LNP MC3), with a value >0.1. Most exemplary lipid nanoparticle compositions (except 5) exhibited a spleen to liver ratio of >1. A few exemplary lipid nanoparticle compositions (LNP 2231, LNP 2291, LNP 2293, LNP 2308, LNP 2339, LNP 2348, LNP 2375, LNP 2376, and LNP 2377) exhibited a spleen to liver ratio of >10. These results indicate that instead of standard delivery mostly by liver exhibited for the comparative lipid nanoparticle compositions, the exemplary lipid nanoparticle compositions exhibited surprising high delivery to spleen delivery in addition to liver delivery.

While this disclosure has been described in relation to some embodiments, and many details have been set forth for purposes of illustration, it will be apparent to those skilled in the art that this disclosure includes additional embodiments, and that some of the details described herein may be varied considerably without departing from this disclosure. This disclosure includes such additional embodiments, modifications, and equivalents. In particular, this disclosure includes any combination of the features, terms, or elements of the various illustrative components and examples.

1. A compound of Formula (I): 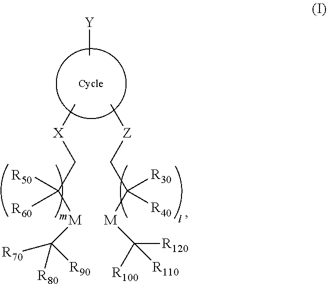
a pharmaceutically acceptable salt thereof, or a stereoisomer of any of the foregoing, wherein: 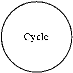
is cyclic or heterocyclic moiety;
Y is alkyl, hydroxy, hydroxyalkyl or 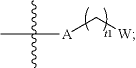
A is absent, —O—, —N(R⁷)—, —O-alkylene-, -alkylene-O—, —OC(O)—, —C(O)O—, —N(R⁷)C(O)—, —C(O)N(R⁷)—, —N(R⁷)C(O)N(R⁷)—, —S—, —S—S—, or a bivalent heterocycle;
each of X and Z is independently absent, —O—, —CO—, —N(R⁷)—, —O-alkylene-; -alkylene-O—, —OC(O)—, —C(O)O—, —N(R⁷)C(O)—, —C(O)N(R⁷)—, or —S—;
each R⁷ is independently H, alkyl, alkenyl, cycloalkyl, hydroxy, hydroxyalkyl, or aminoalkyl;
each M is independently a biodegradable moiety;
each of R₃₀, R₄₀, R₅₀, R₆₀, R₇₀, R₈₀, R₉₀, R₁₀₀, R₁₁₀, and R₁₂₀ is independently H, C₁-C₁₆ branched or unbranched alkyl or C₁-C₁₆ branched or unbranched alkenyl, optionally interrupted with heteroatom or substituted with OH, SH, or halogen, or cycloalkyl or substituted cycloalkyl;
each of l and m is an integer from 1 to 10;
t1 is an integer from 0 to 10; and
W is hydroxyl, substituted or unsubstituted hydroxyalkyl, substituted or unsubstituted amino, substituted or unsubstituted aminocarbonyl, or substituted or unsubstituted heterocyclyl or heteroaryl.

2. (canceled)

3. The compound of claim 1, having the structure: 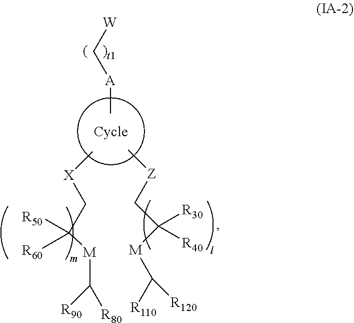
wherein: 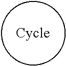
is selected from the group consisting of 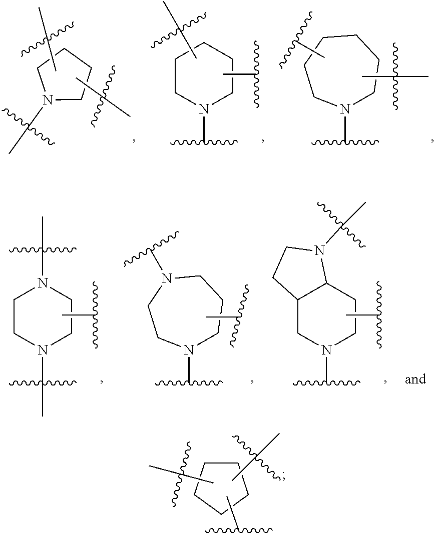
A is absent, —O—, —N(R⁷)—, —N(R⁷)C(O)—, 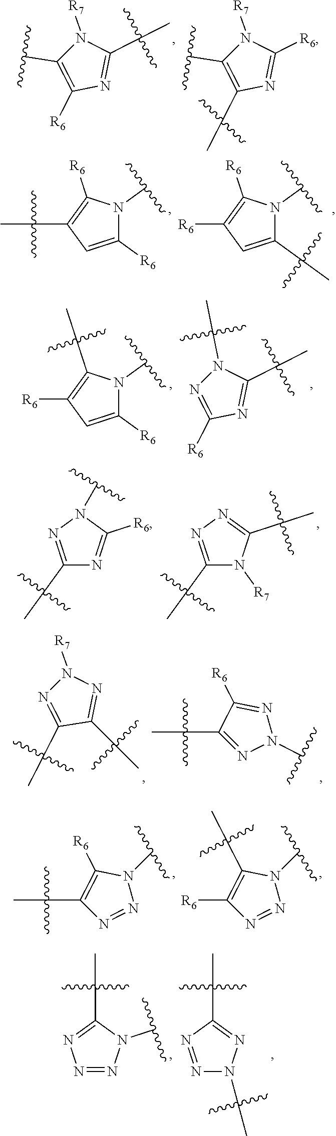
—OC(O)—, or —C(O)O—, wherein R⁶ is independently H, alkyl, hydroxyl, hydroxyalkyl, amino, aminoalkyl, thiol, thiolalkyl, or N⁺(R⁷)₃-alkylene-Q-; and R⁷ is H or C₁-C₃ alkyl:
X is absent, —O—, or —C(O)—;
Z is —O—, —C(O)O—, or —OC(O)—;
each of R₃₀, R₄₀, R₅₀, and R₆₀ is H or C₁-C₄ branched or unbranched alkyl;
R₇₀ is H; and each of R₈₀ and R₉₀ is independently H or C₁-C₁₂ branched or unbranched alkyl; and R₁₀₀ is H; and ach of R₁₁₀ and R₁₂₀ is independently H or C₁-C₁₂ branched or unbranched alkyl, provided that at least one of R₈₀ and R₉₀ is not H, and at least one of R₁₁₀ and R₁₂₀ is not H;
M is —OC(O)—, —C(O)O—, —N(R^{7′})C(O)—, —C(O)N(R⁷)—, —C(O—R₁₃)—O—, —C(O)O(CH₂)ᵣ—, —C(O)N(R⁷) (CH₂)ᵣ—, or —C(O—R₁₃)—O—(CH₂)ᵣ—, wherein each R⁷ is independently H, alkyl, alkenyl, cycloalkyl, hydroxyalkyl, or aminoalkyl; R₁₃ is branched or unbranched C₃-C₁₀ alkyl, and r is 1, 2, 3, 4, or 5;
l is an integer from 3 to 7;
m is an integer from 1 to 5;
t1 is 0, 1, 2, 3 or 4; and/or
W is hydroxyl, hydroxyalkyl, or one of the following moieties: 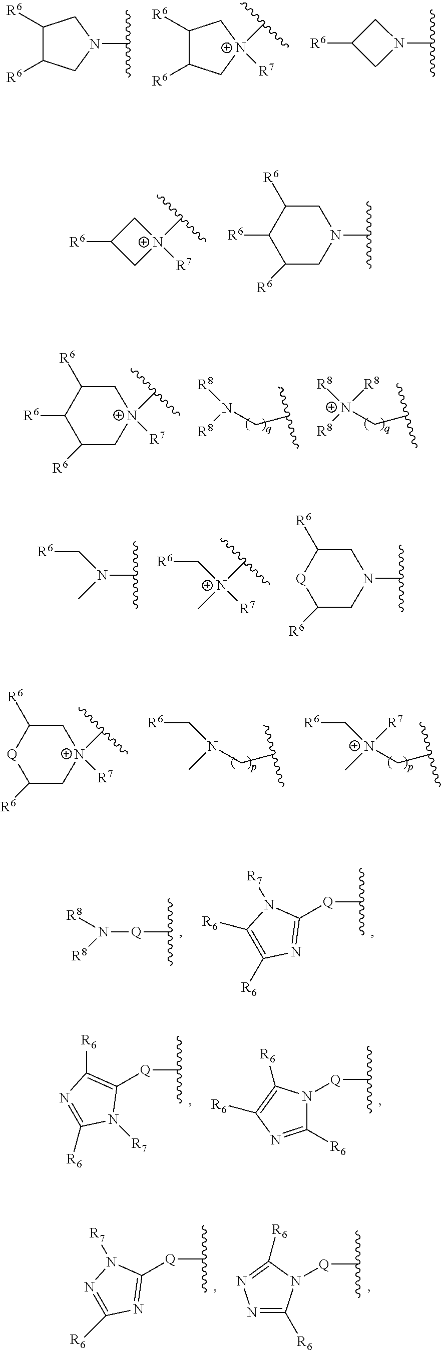 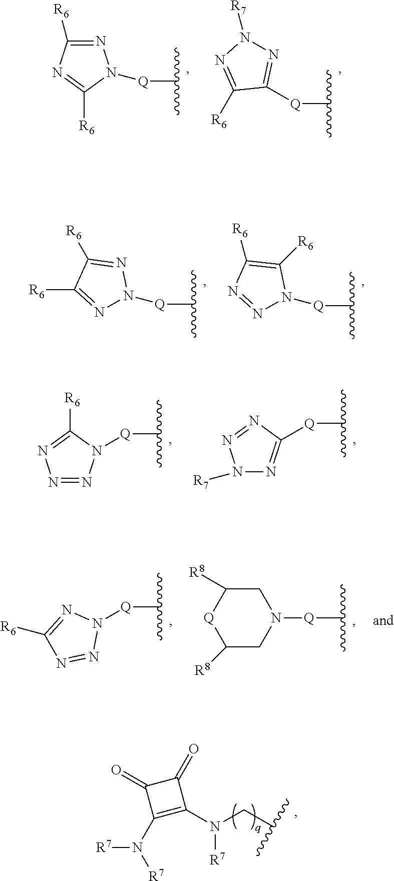
wherein:
each Q is independently absent, —O—, —C(O)—, —C(S)—, —C(O)O—, —C(R⁷)₂—, —C(O)N(R⁷)—, —C(S)N(R⁷)—, or —N(R⁷)—;
each R⁶ is independently H, alkyl, hydroxyl, hydroxyalkyl, alkoxy, amino, aminoalkyl, alkylamino, thiol, thiolalkyl, or N⁺(R⁷)₃-alkylene-Q-;
each R⁸ is independently H, alkyl, hydroxyalkyl, amino, aminoalkyl, thiol, or thiolalkyl, or two R⁸ together with the nitrogen atom may form a ring;
each q is independently 0, 1, 2, 3, 4, or 5; and
each p is independently 0, 1, 2, 3, 4, or 5.

4-20. (canceled)

21. The compound of claim 3, wherein: 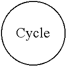
is selected from the group consisting of 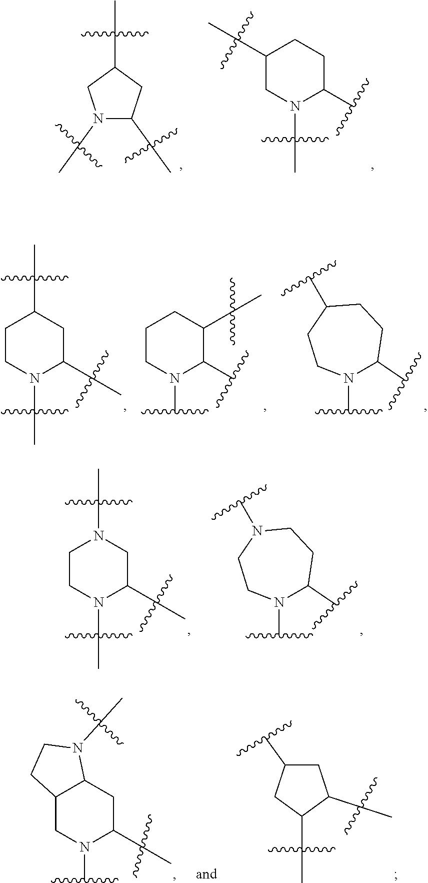
M is —OC(O)— or —C(O)O—;
each of R₃₀, R₄₀, R₅₀, and R₆₀ is H; 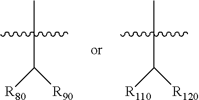
or is independently selected from the group consisting of 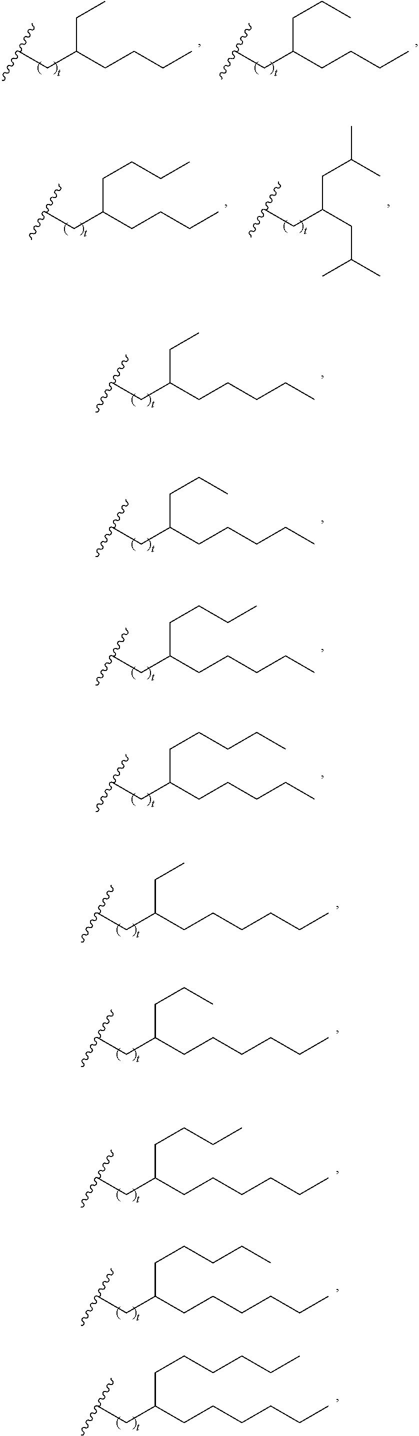 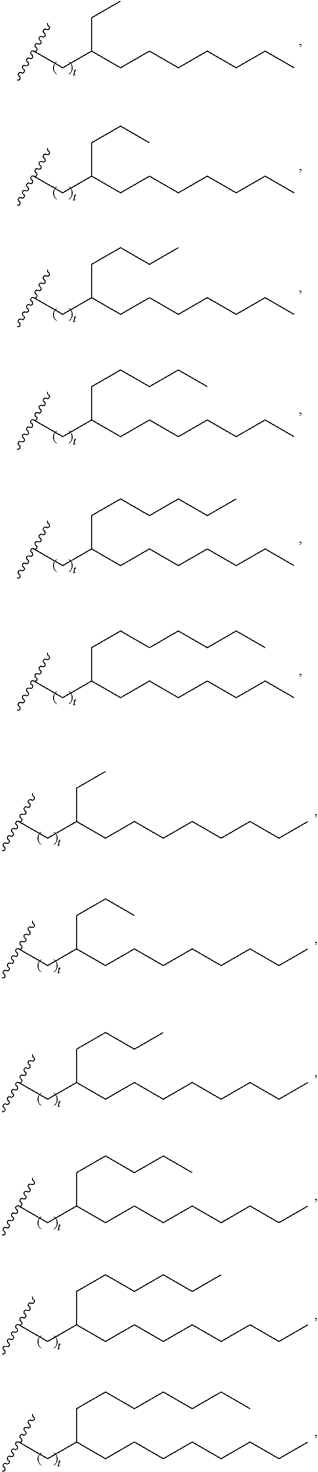 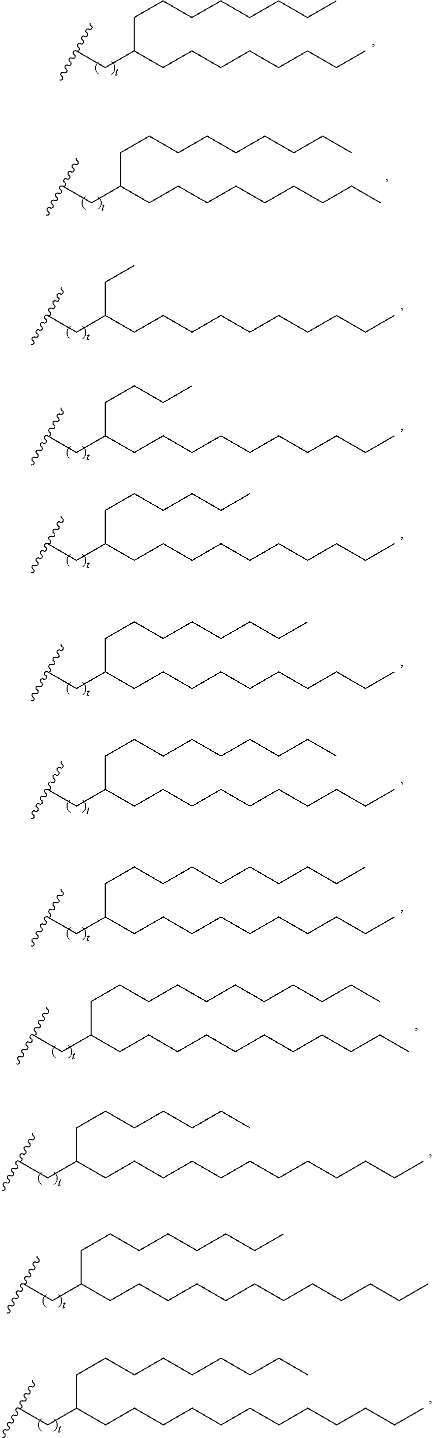 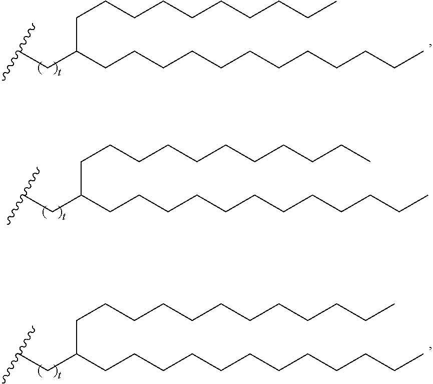
wherein t is 0, 1, 2, 3, 4, or 5; and/or
W is OH, 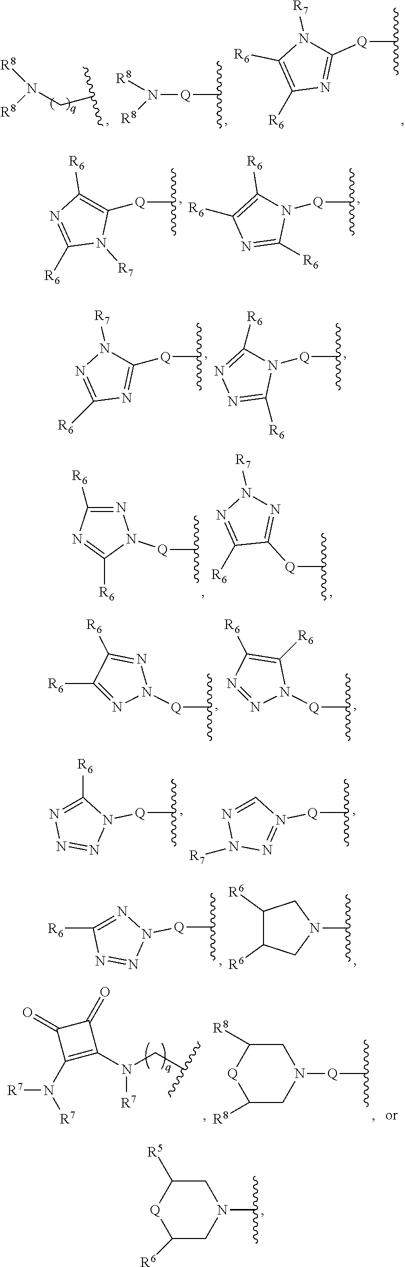
wherein
q is 0,
each R⁸ is independently H, C₁-C₃ alkyl, or hydroxyalkyl, or two R⁸ together with the nitrogen atom form a 5-membered ring optionally substituted with one or more alkyl groups,
each R⁶ is independently H, hydroxyl, hydroxyalkyl, alkoxy, amino, aminoalkyl, alkylamino, C₁-C₃ alkyl, or -Q-alkylene-N⁺(R⁷)₃,
each Q is independently absent, —O—, —C(O)—, —N(R⁷)—, —C(R⁷)₂—, —C(O)O—, —C(O)N(R⁷)—, or —C(S)N(R⁷)—, and
each R⁷ is independently H, C₁-C₃ alkyl, hydroxy, or hydroxyalkyl.

22. The compound of claim 3, wherein W is OH,

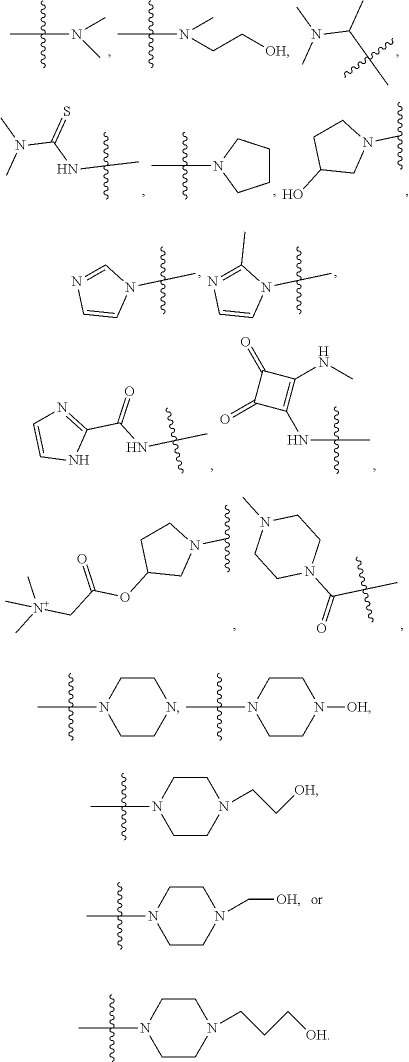

23. The compound of claim 3, wherein Y or 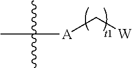
is:
OH, 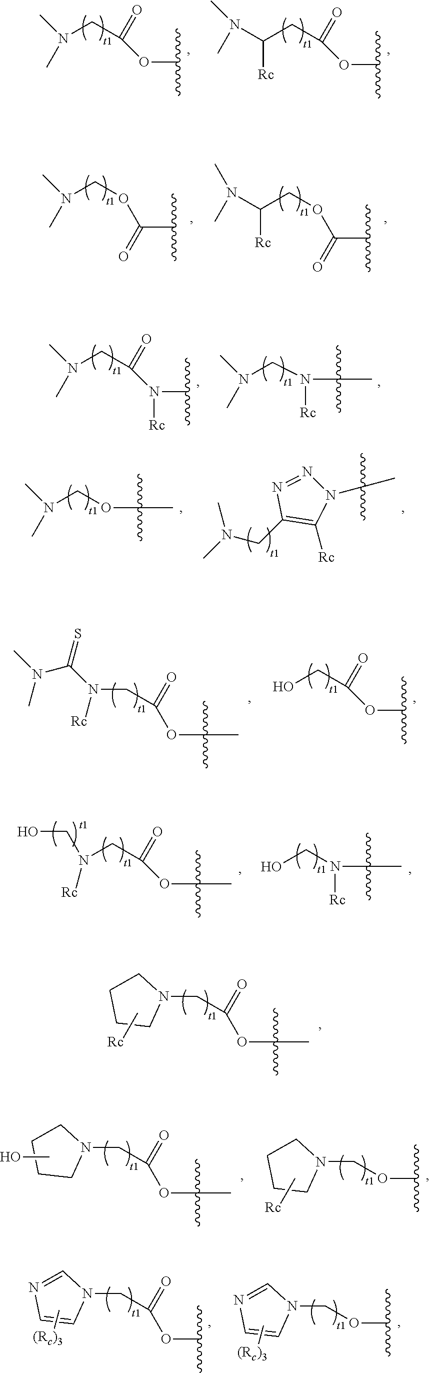 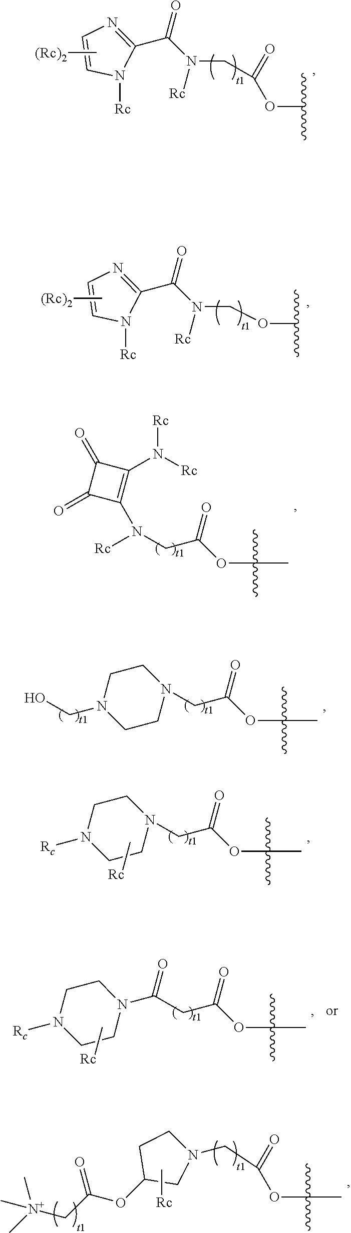
wherein each R^{c} is independently H or C₁-C₃ alkyl, and each t1 is independently 1, 2, 3, or 4.

24. The compound of claim 3, wherein Y or 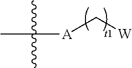
is: OH,

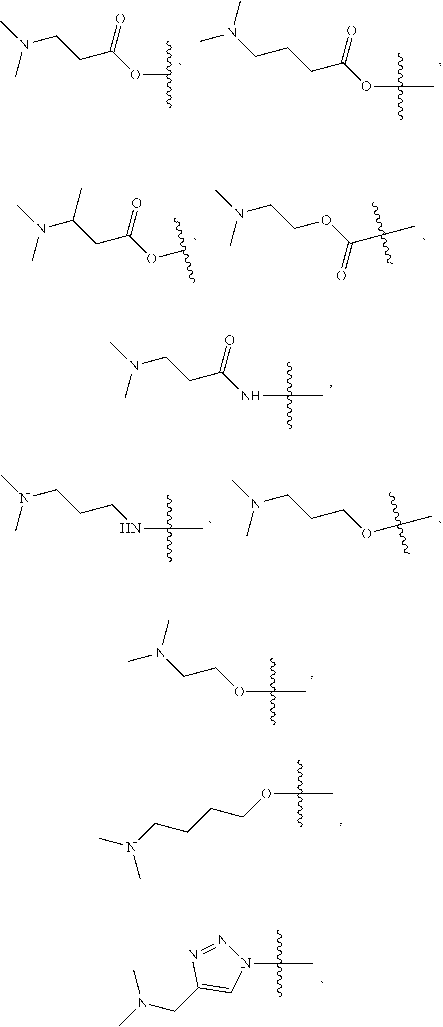

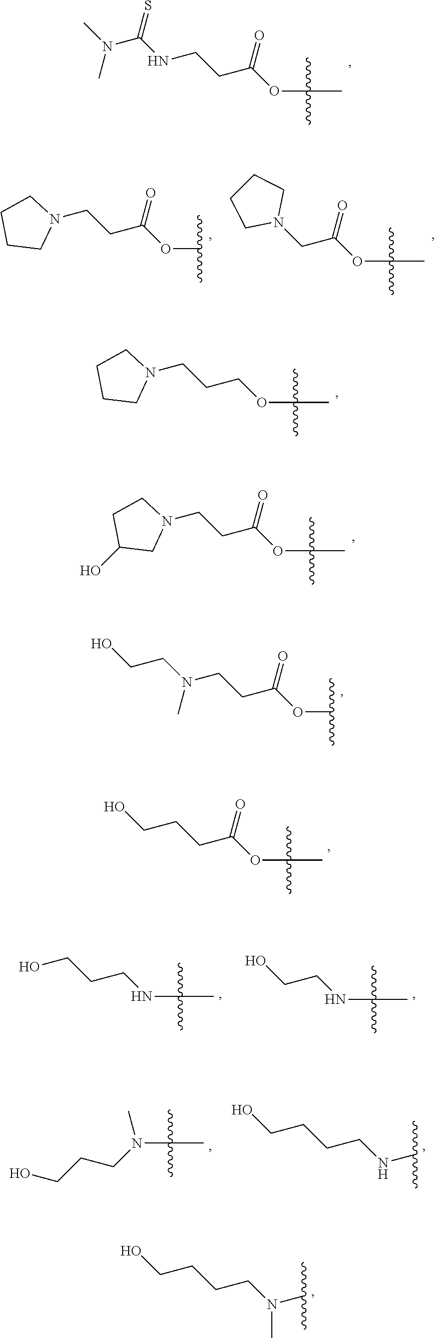

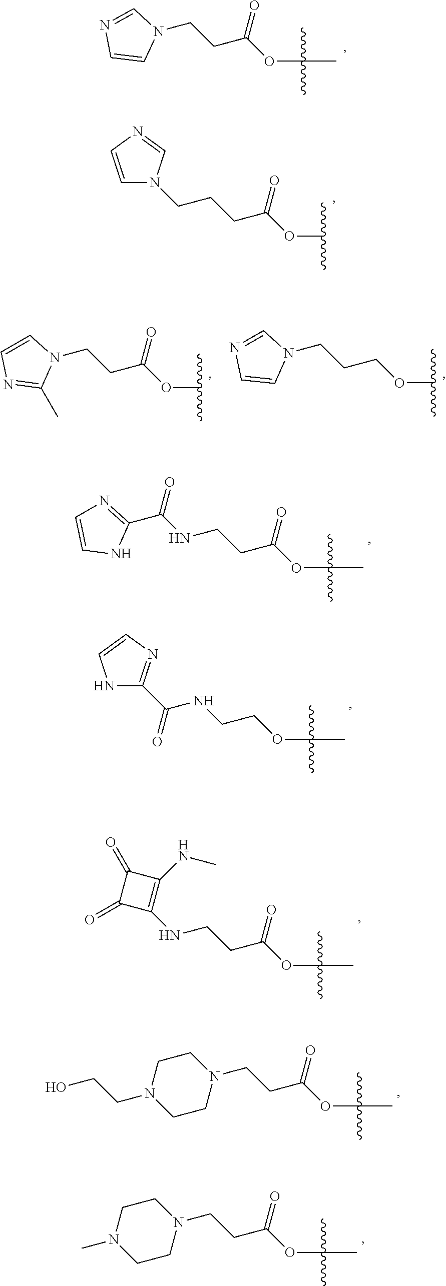

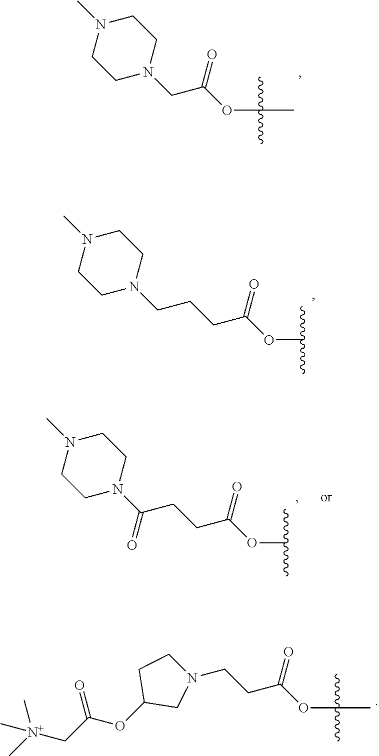 [SMILES: *OC(=O)CN1CCN(C)CC1 ; *OC(=O)CCCN1CCN(C)CC1 ; *OC(=O)CCC(=O)N1CCN(C)CC1 ; *OC(=O)CCN1CCC(OC(=O)C[N+](C)(C)C)C1]

25. The compound of claim 3, wherein:
X is absent, —O—, or —C(O)—;
Z is —O—, —C(O)O—, or —OC(O)—;
M is —OC(O)— or —C(O)O—;
Y or is: 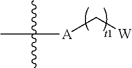
is:
OH, 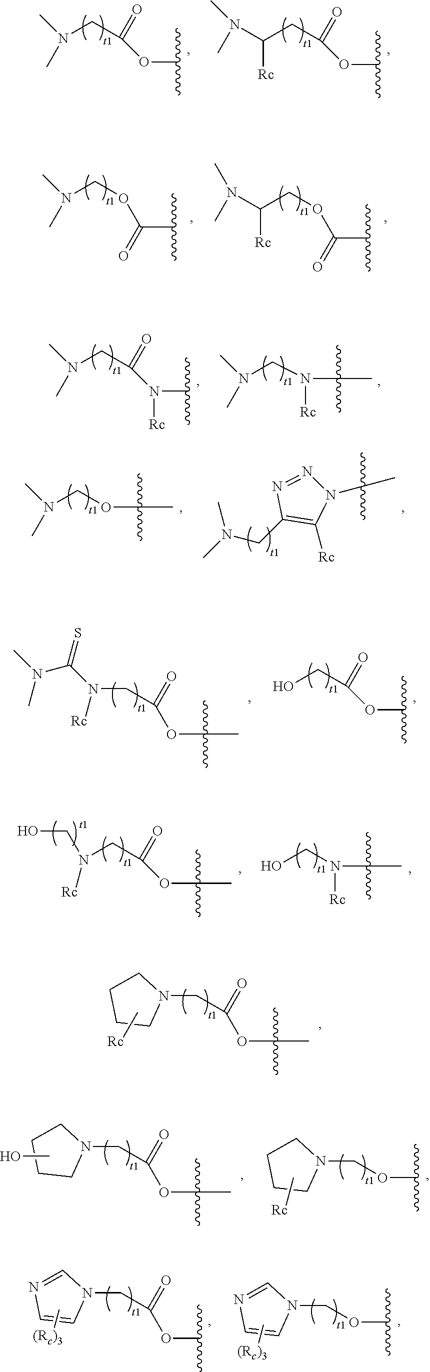 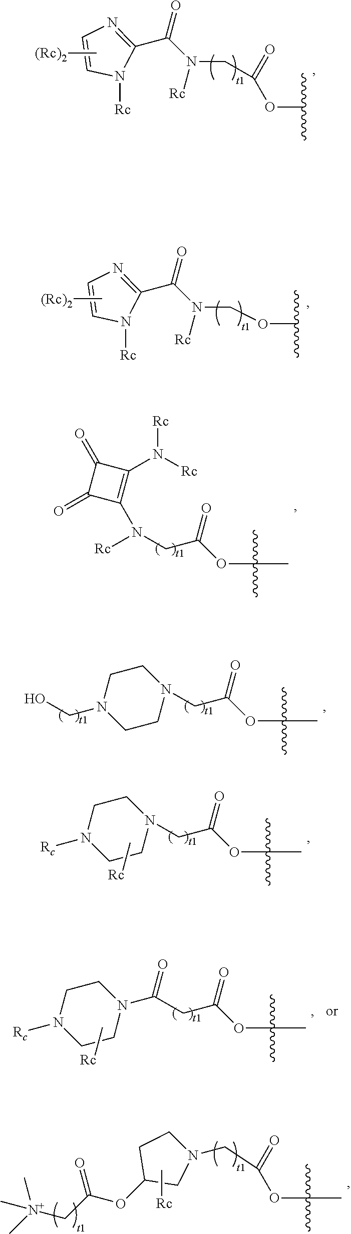
each R^{c} is independently H or C₁-C₃ alkyl;
each t1 is independently 1, 2, 3, or 4;
each of R₃₀, R₄₀, R₅₀, and R₆₀ is H or C₁-C₄ branched or unbranched alkyl;
R₇₀ is H; and each of R₅₀ and R₉₀ is independently H or C₁-C₁₂ branched or unbranched alkyl;
R₁₀₀ is H; and each of R₁₁₀ and R₁₂₀ is independently H or C₁-C₁₂ branched or unbranched alkyl, provided that at least one of R₈₀ and R₉₀ is not H, and at least one of R₁₁₀ and R₁₂₀ is not H;
l is from 3 to 7; and
m is from 1 to 5.

26. (canceled)

27. The compound of claim 1, having the formula: 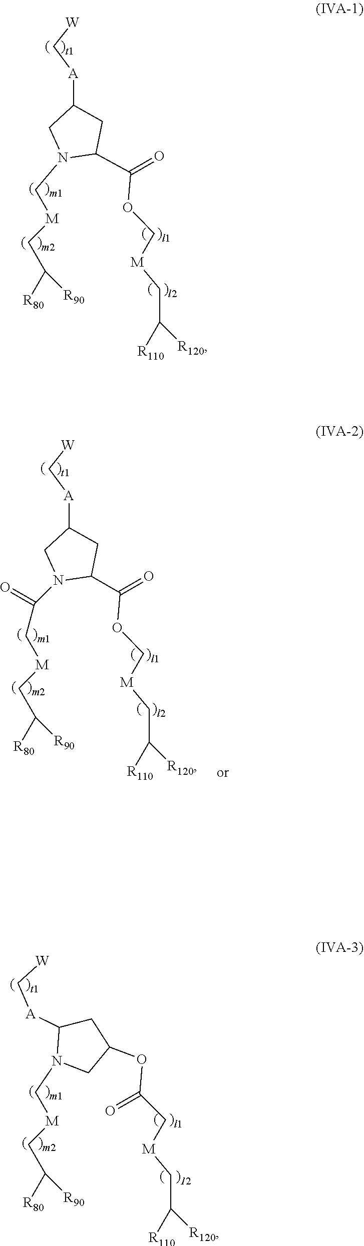
wherein:
each m1 is independently an integer from 3 to 6,
each 11 is independently an integer from 4 to 8,
m2 and 12 are each independently an integer from 0 to 3,
R₈₀ and R₉₀ are each independently unsubstituted C₅-C₈ alkyl; or R₈₀ is H or unsubstituted C₁-C₄ alkyl, and R₉₀ is unsubstituted C₅-C₁₁ alkyl; and
R₁₁₀ and R₁₂₀ are each independently unsubstituted C₅-C₈ alkyl; or R₁₁₀ is H or unsubstituted C₁-C₄ alkyl, and R₁₂₀ is unsubstituted C₅-C₁₁ alkyl.

28. The compound of claim 27, having the formula: 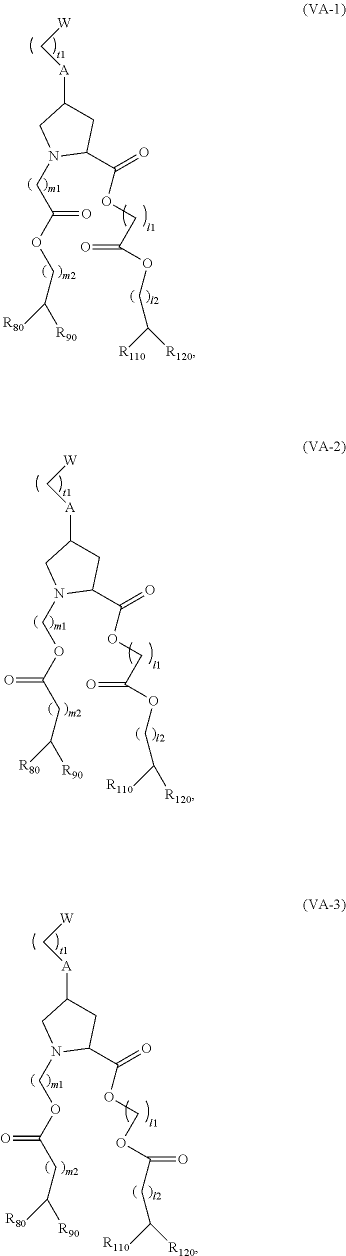 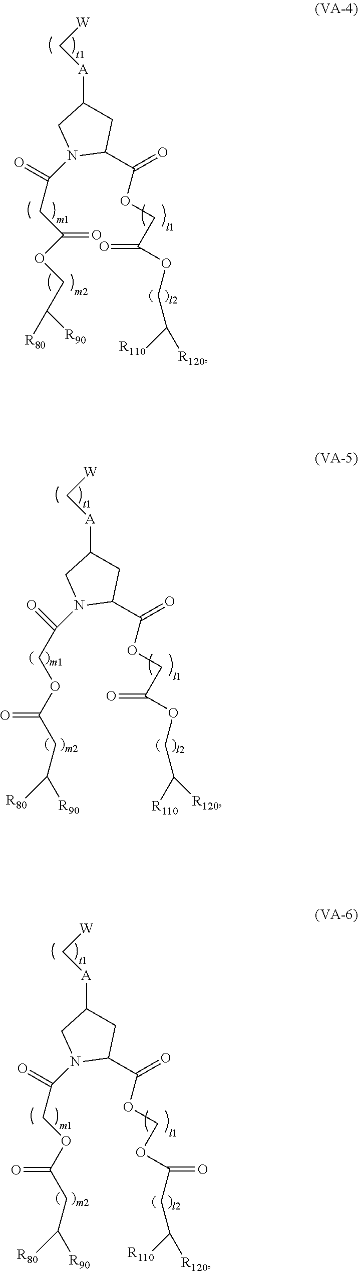 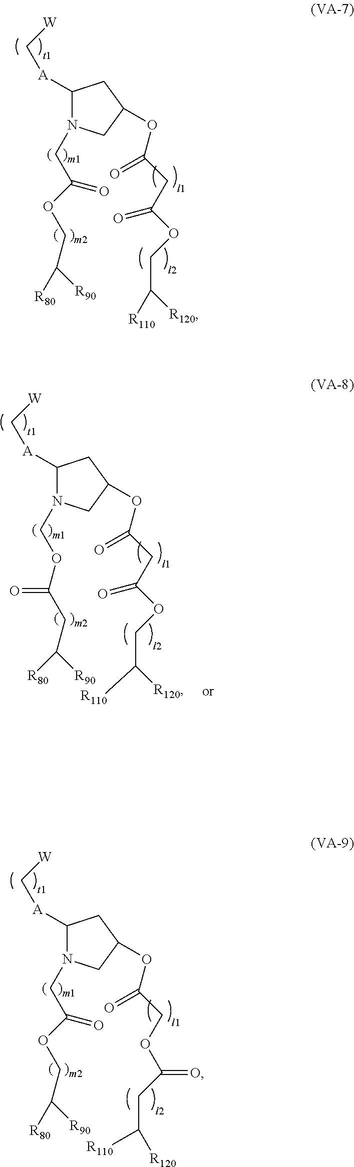
wherein 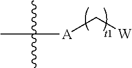
is:
OH,

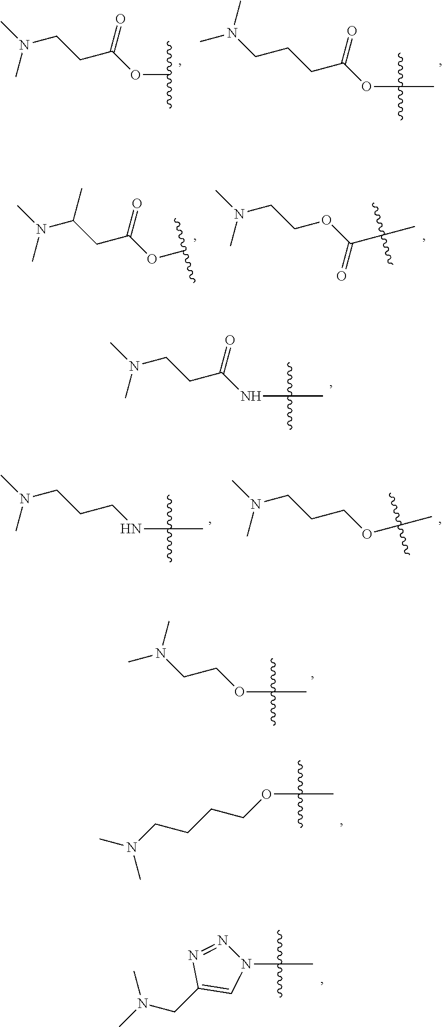

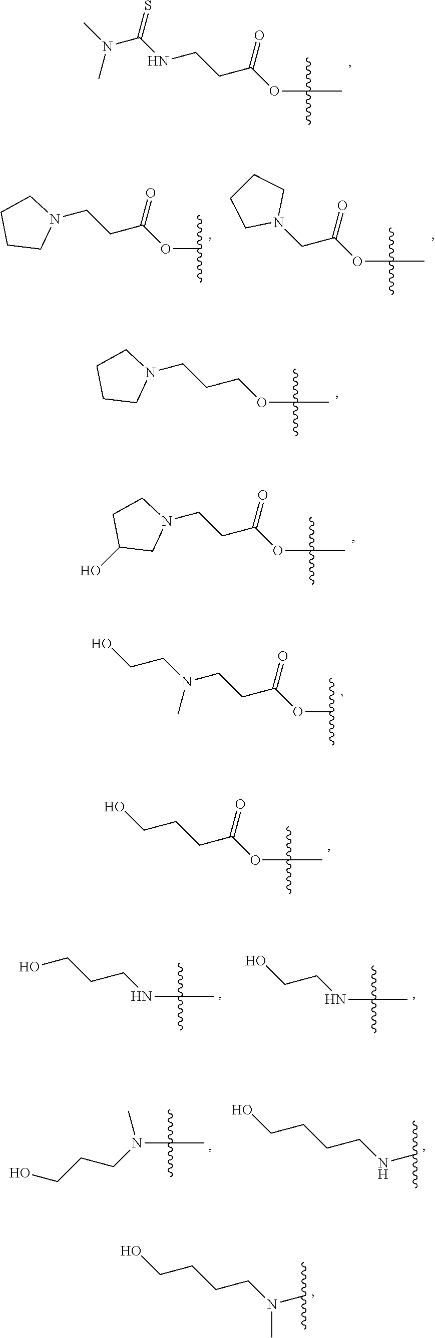

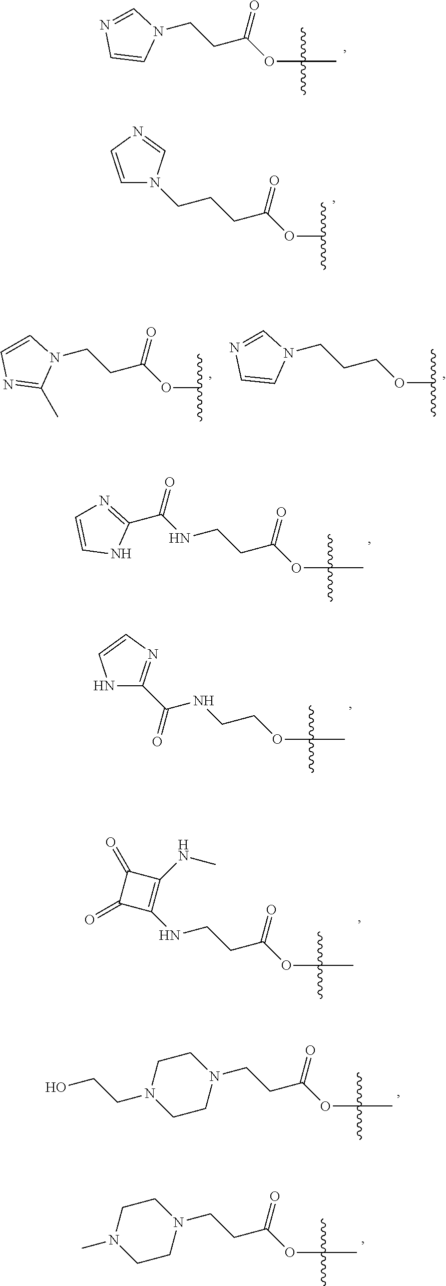

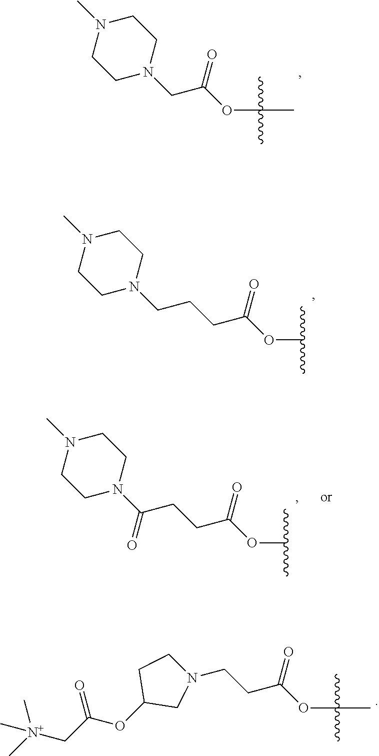 [SMILES: *OC(=O)CN1CCN(C)CC1 ; *OC(=O)CCCN1CCN(C)CC1 ; *OC(=O)CCC(=O)N1CCN(C)CC1 ; *OC(=O)CCN1CCC(OC(=O)C[N+](C)(C)C)C1]

29. The compound of claim 28, wherein
R₈₀ is H or unsubstituted C₁-C₂ alkyl, and R₉₀ is unsubstituted C₆-C₁₀ alkyl; and
R₁₁₀ and R₁₂₀ are each independently unsubstituted C₅-C₈ alkyl; or
R₈₀, R₉₀, R₁₁₀, and R₁₂₀ are each independently unsubstituted C₅-C₈alkyl.

30-31. (canceled)

32. The compound of claim 1, having the formula: 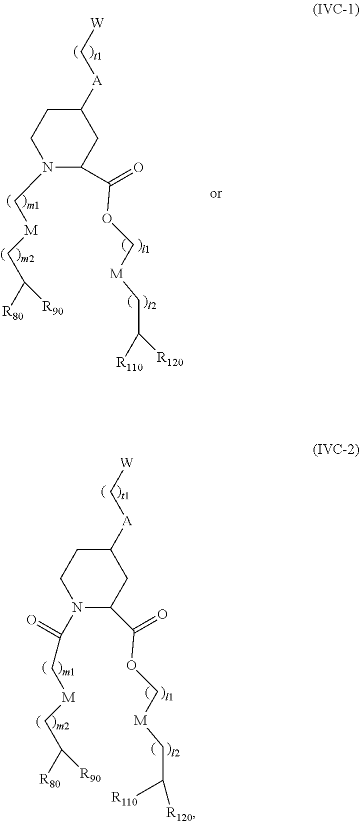
wherein:
each m1 is independently an integer from 3 to 6,
each 11 is independently an integer from 4 to 8,
m2 and 12 are each independently an integer from 0 to 3,
R₈₀ and R₉₀ are each independently unsubstituted C₅-C₈ alkyl; or R₈₀ is H or unsubstituted C₁-C₄ alkyl, and R₉₀ is unsubstituted C₅-C₁₁ alkyl; and
R₁₁₀ and R₁₂₀ are each independently unsubstituted C₅-C₈ alkyl; or R₁₁₀ is H or unsubstituted C₁-C₄ alkyl, and R₁₂₀ is unsubstituted C₅-C₁₁ alkyl.

33. The compound of claim 32, having the formula: 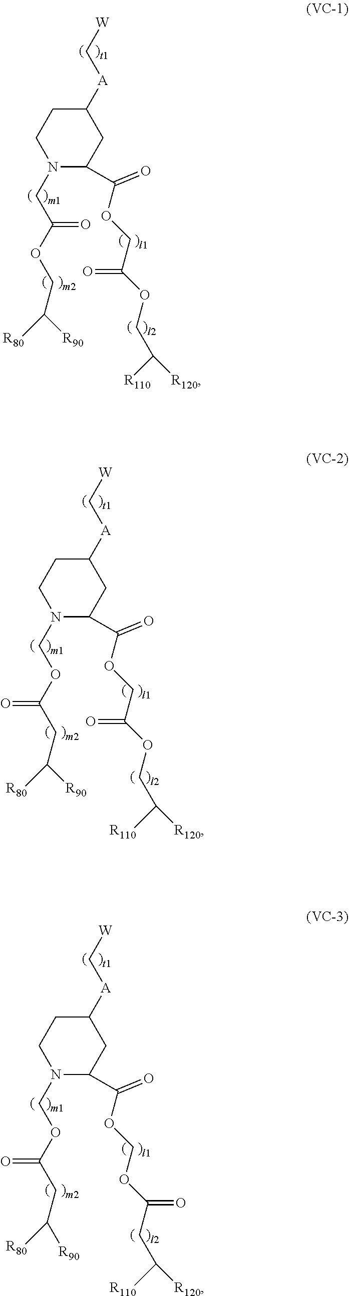 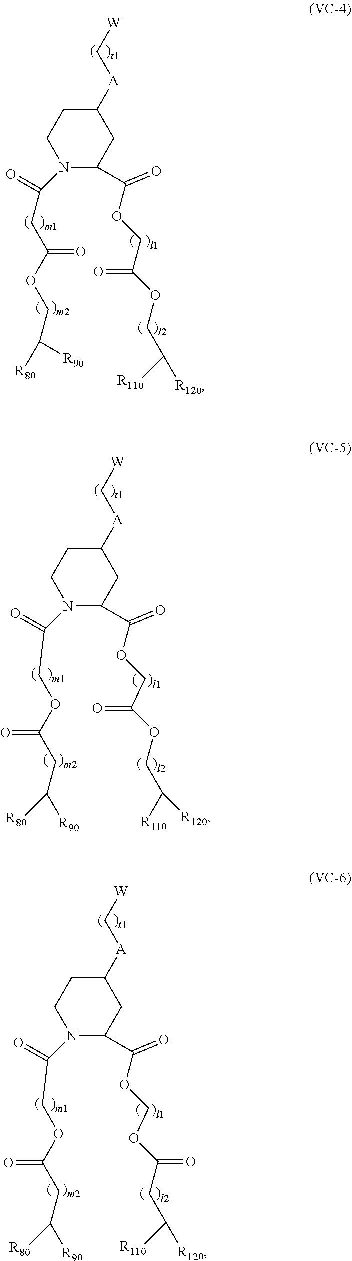
wherein 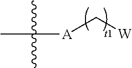
is:
OH,

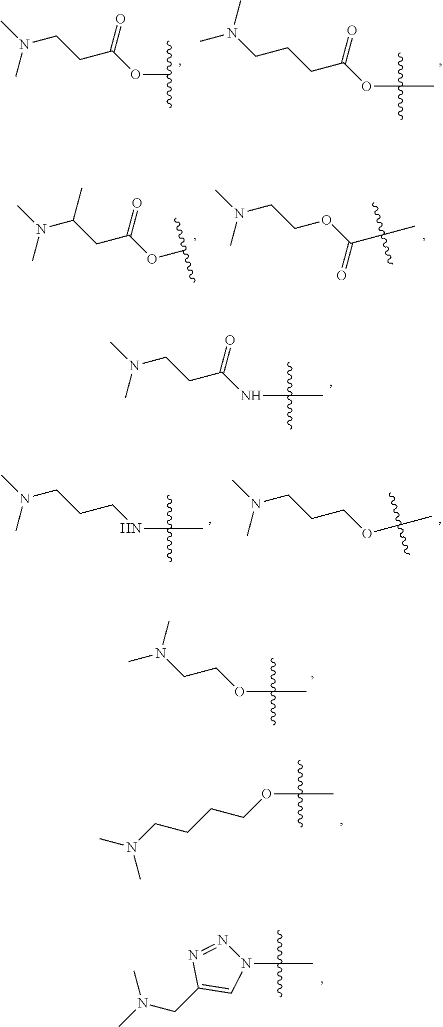

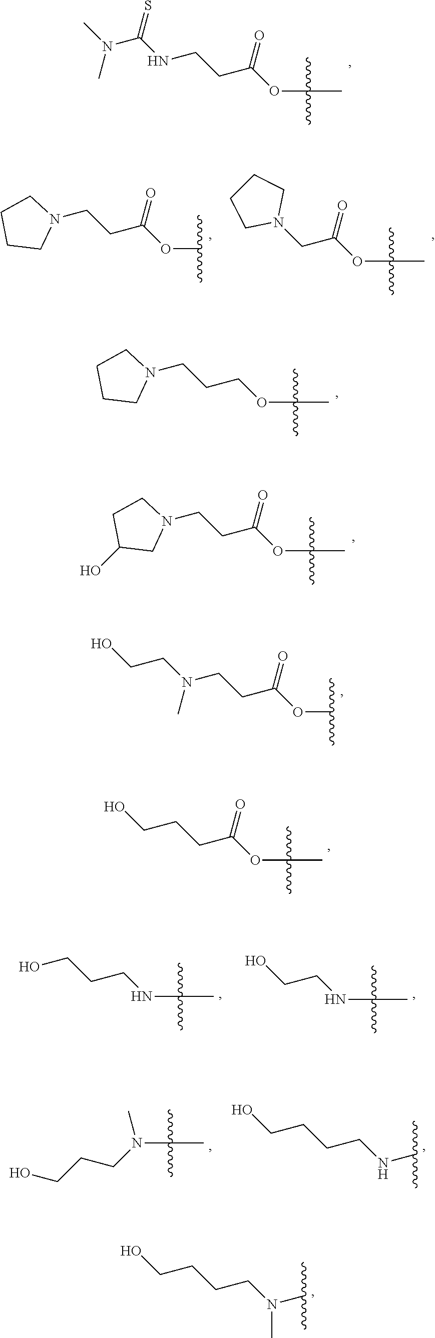

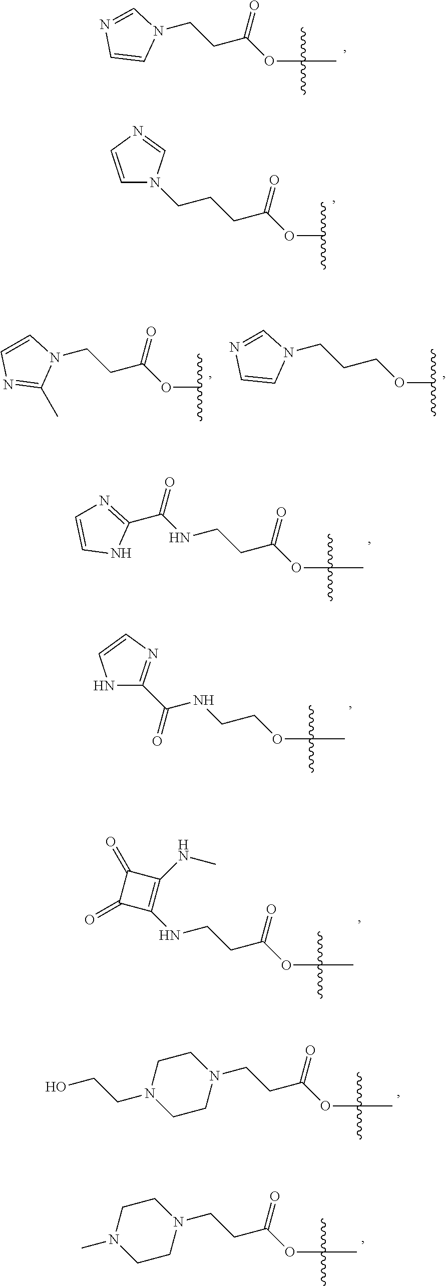

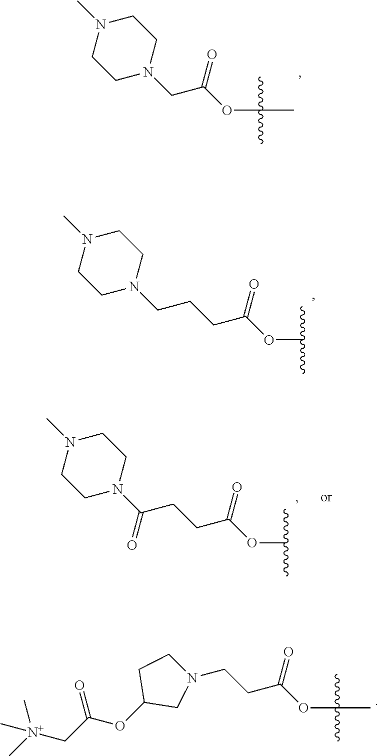 [SMILES: *OC(=O)CN1CCN(C)CC1 ; *OC(=O)CCCN1CCN(C)CC1 ; *OC(=O)CCC(=O)N1CCN(C)CC1 ; *OC(=O)CCN1CCC(OC(=O)C[N+](C)(C)C)C1]

34. The compound of claim 33, wherein
R₈₀ is H or unsubstituted C₁-C₂ alkyl, and R₉₀ is unsubstituted C₆-C₁₀ alkyl; and
R₁₁₀ and R₁₂₀ are each independently unsubstituted C₅-C₈ alkyl; or
R₈₀, R₉₀, R₁₁₀, and R₁₂₀ are each independently unsubstituted C₅-C₈ alkyl.

35-37. (canceled)

38. The compound of claim 1, having one of the following structures:

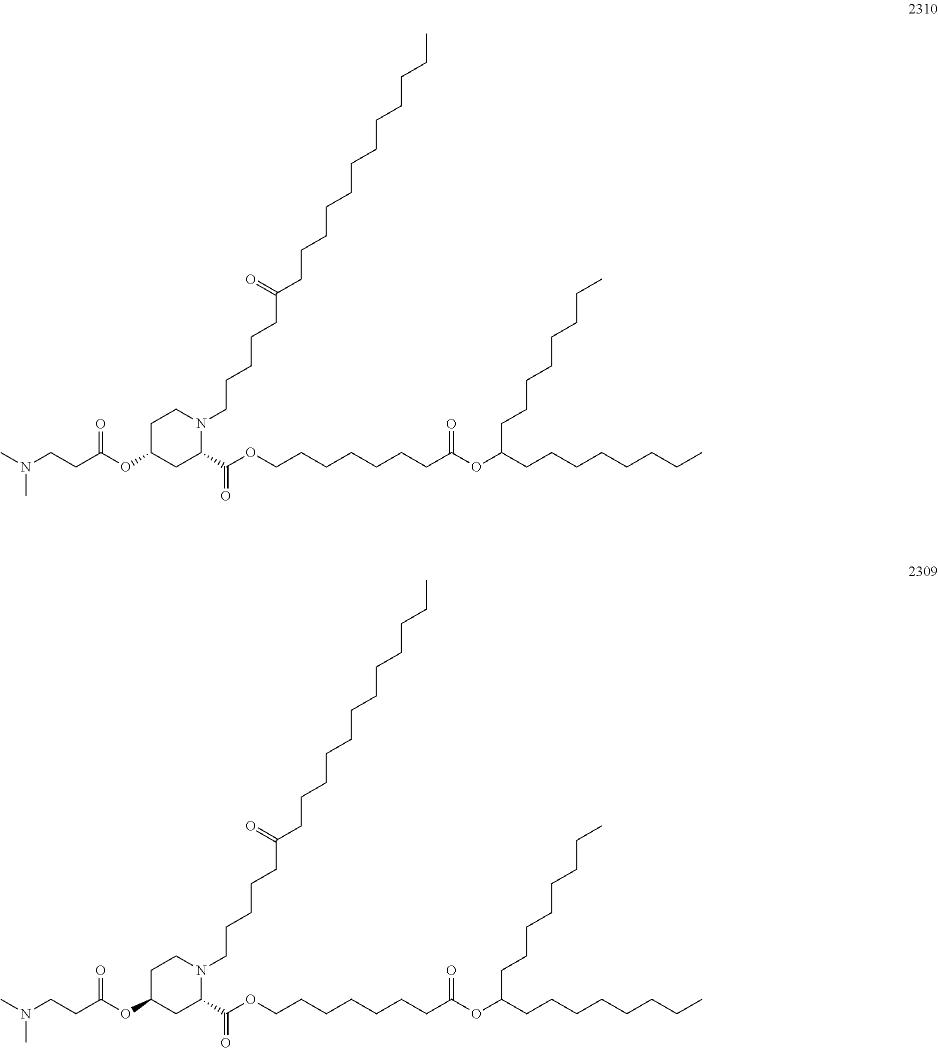

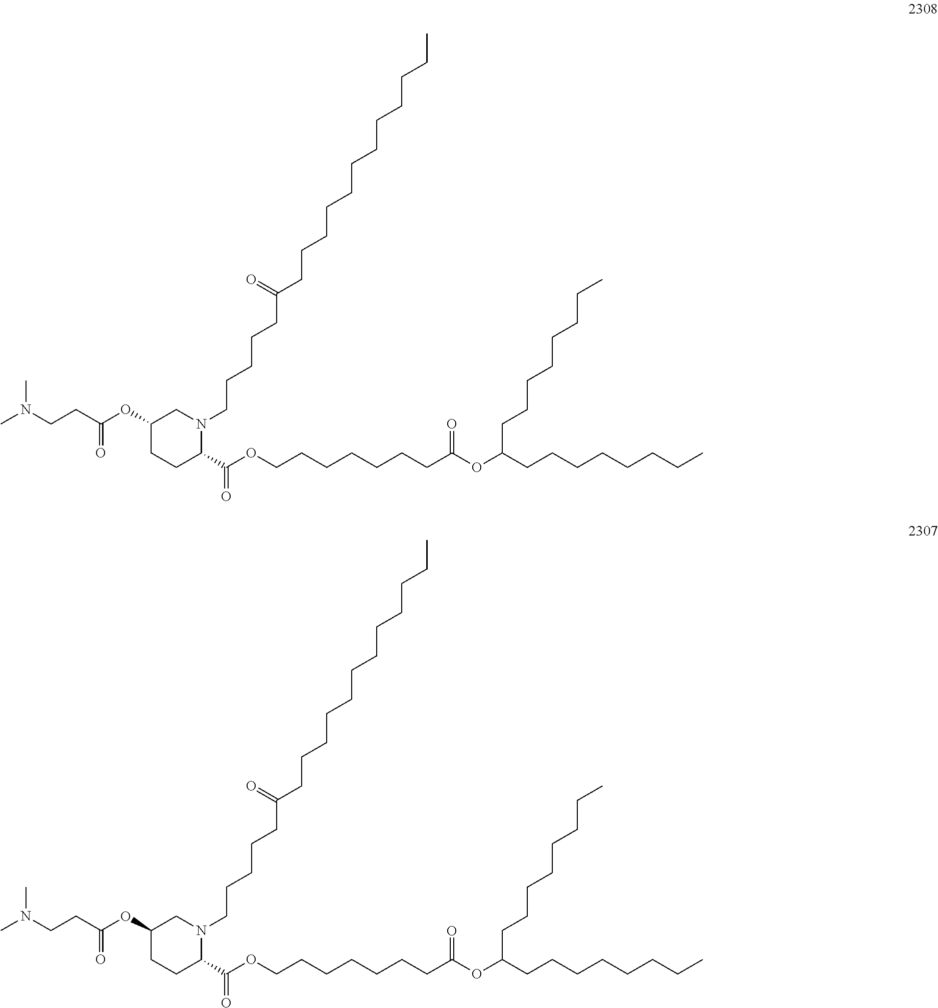

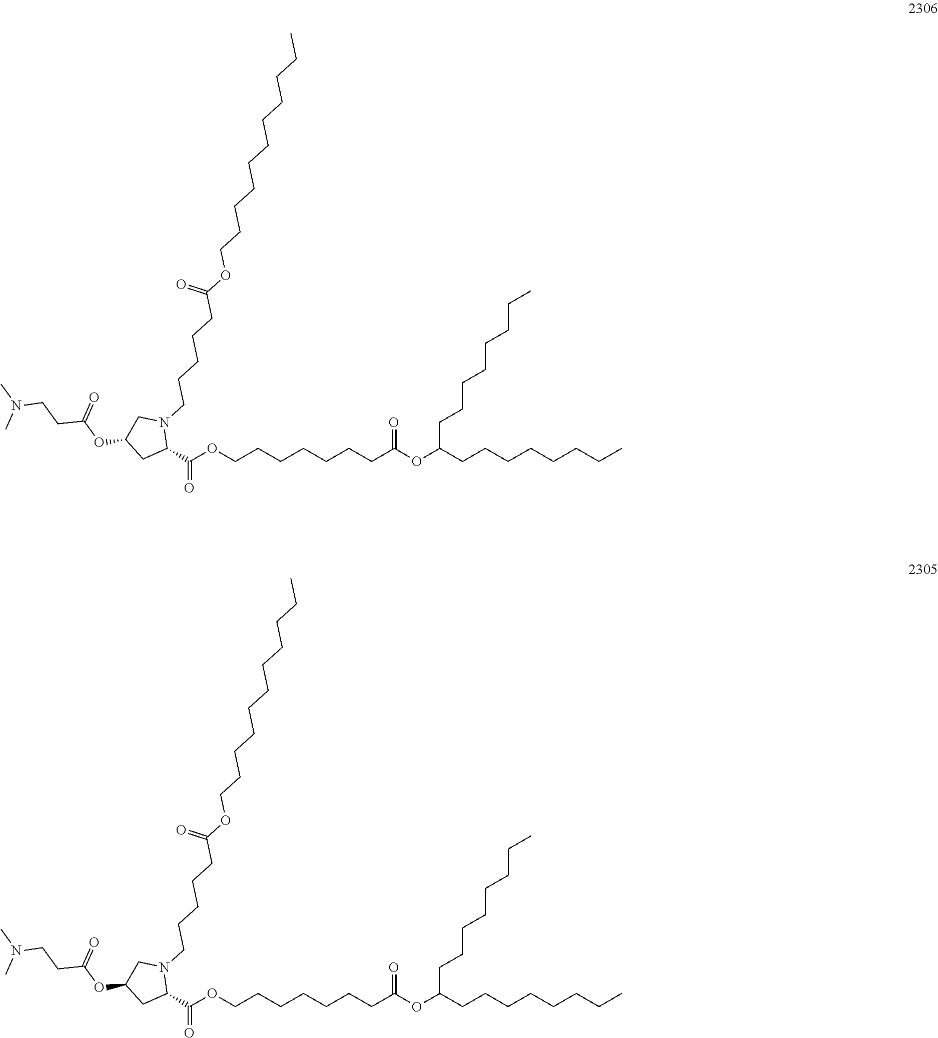

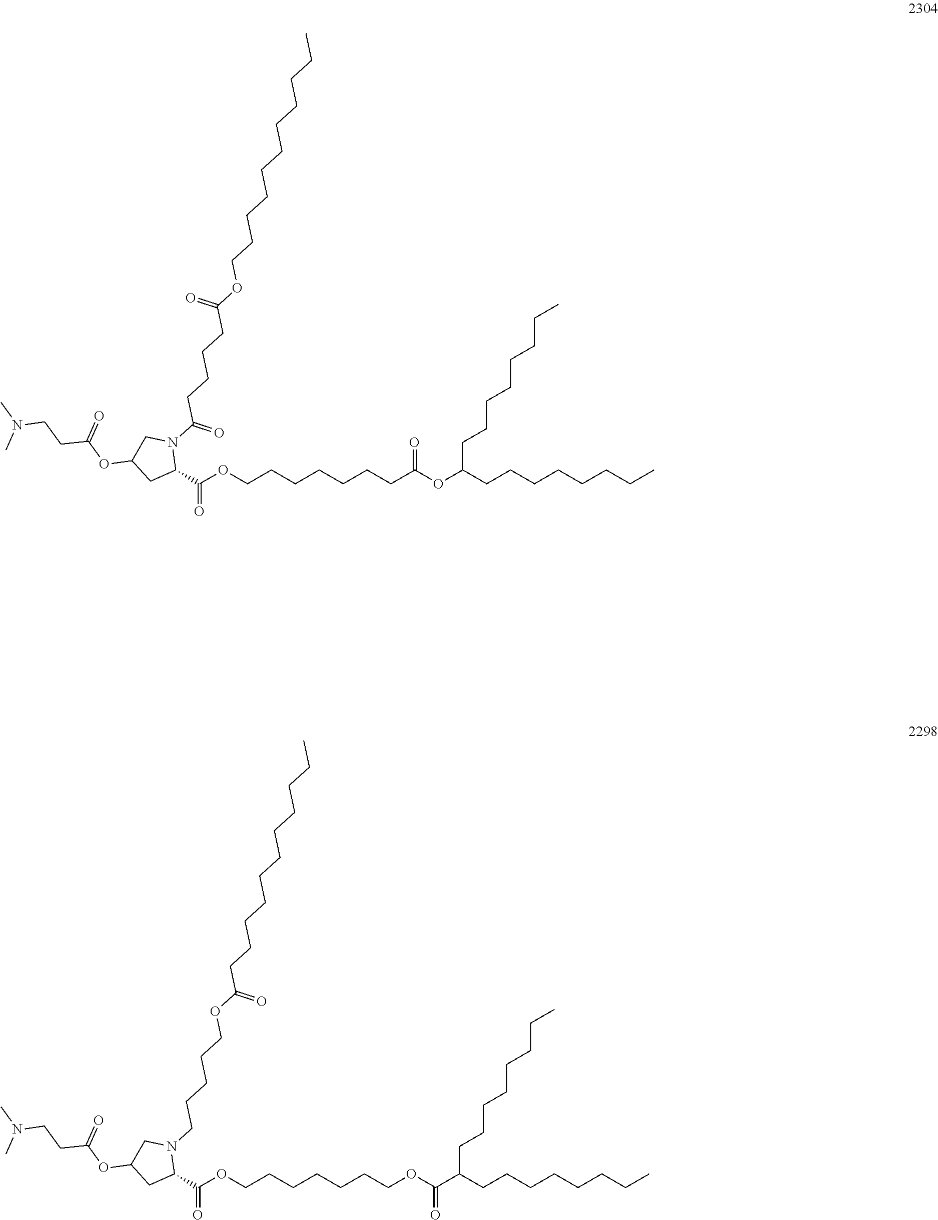

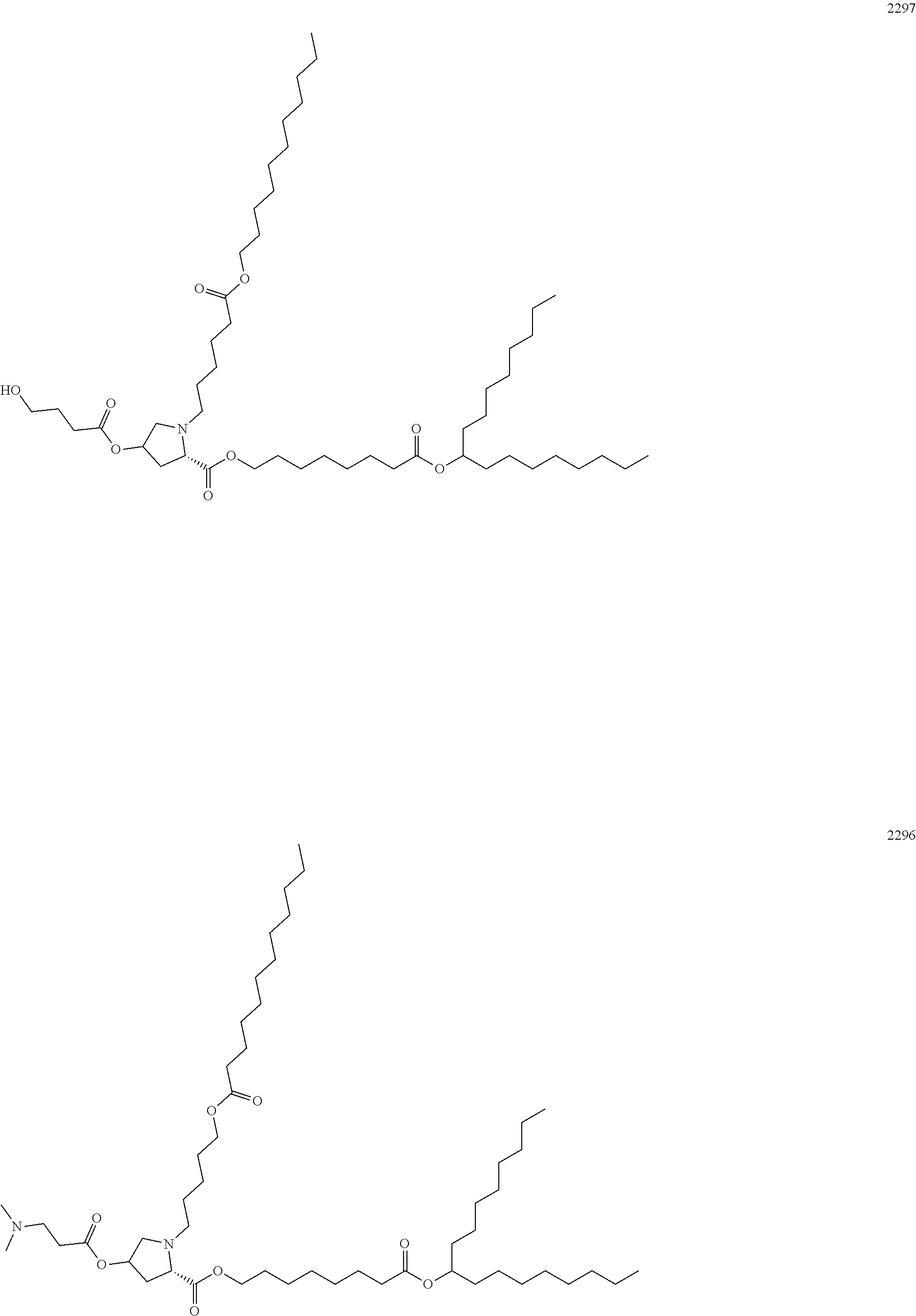

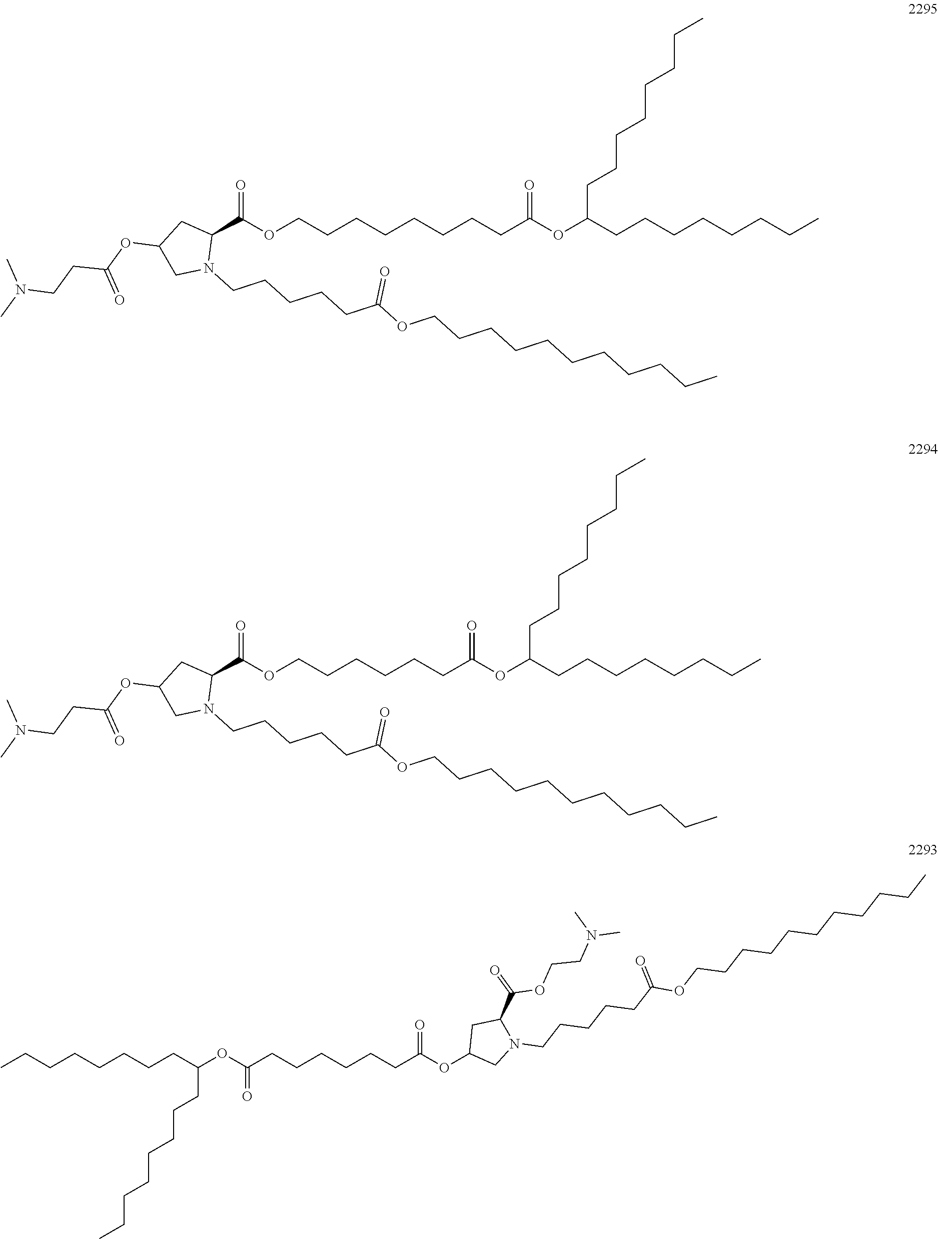

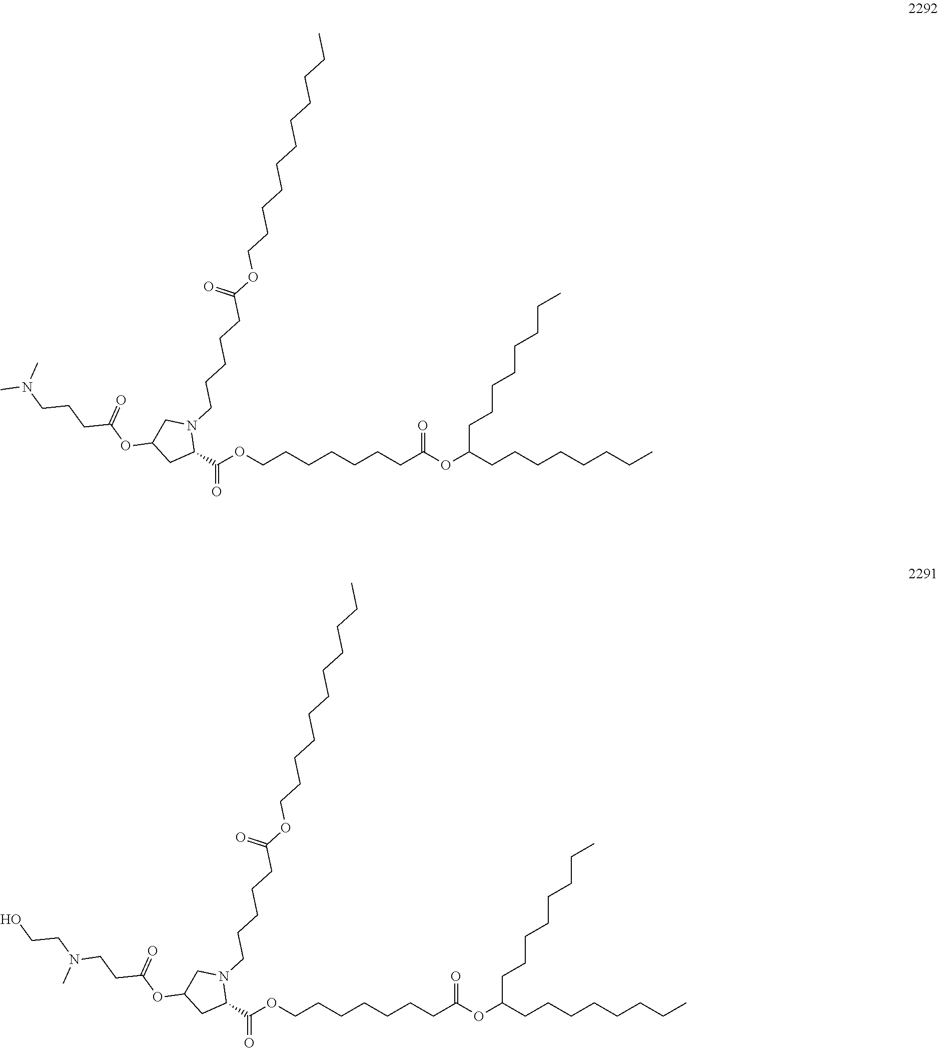 [SMILES: CCCCCCCCCCCOC(=O)CCCCCN1CC(OC(=O)CCCN(C)C)C[C@H]1C(=O)OCCCCCCCC(=O)OC(CCCCCCCC)CCCCCCCC ; CCCCCCCCCCCOC(=O)CCCCCN1CC(OC(=O)CCN(C)CCO)C[C@H]1C(=O)OCCCCCCCC(=O)OC(CCCCCCCC)CCCCCCCC]

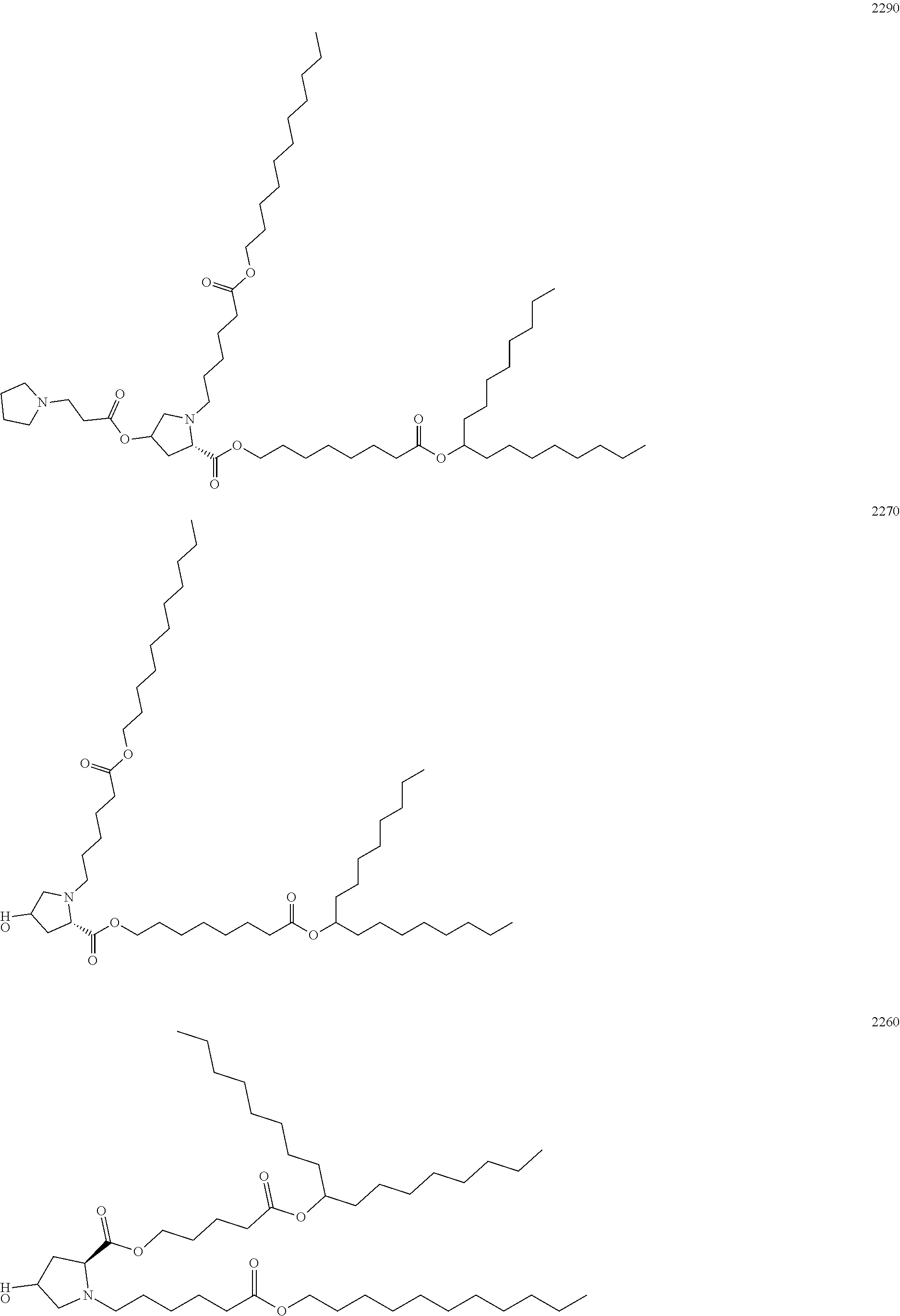 [SMILES: CCCCCCCCCCCOC(=O)CCCCCN1CC(OC(=O)CCN2CCCC2)C[C@H]1C(=O)OCCCCCCCC(=O)OC(CCCCCCCC)CCCCCCCC ; CCCCCCCCCCCOC(=O)CCCCCN1CC(O)C[C@H]1C(=O)OCCCCCCCC(=O)OC(CCCCCCCC)CCCCCCCC ; CCCCCCCCCCCOC(=O)CCCCCN1CC(O)C[C@H]1C(=O)OCCCCC(=O)OC(CCCCCCCC)CCCCCCCC]

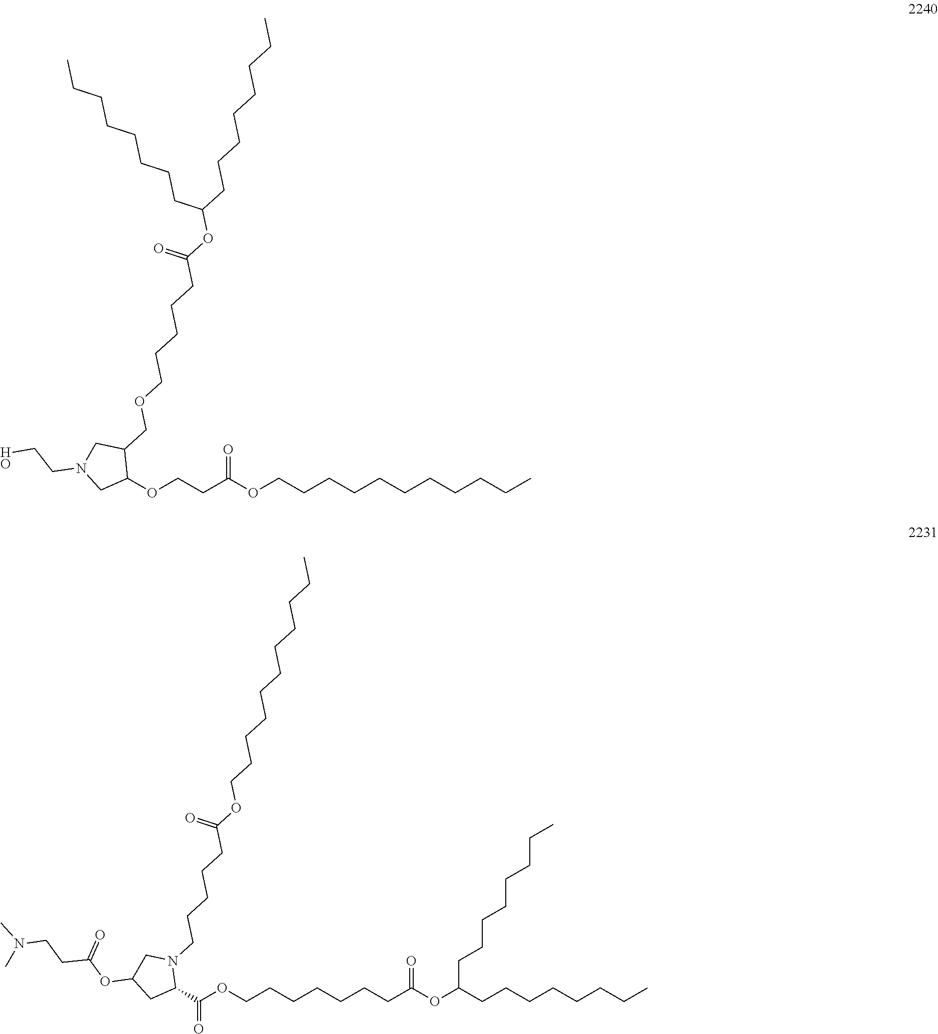

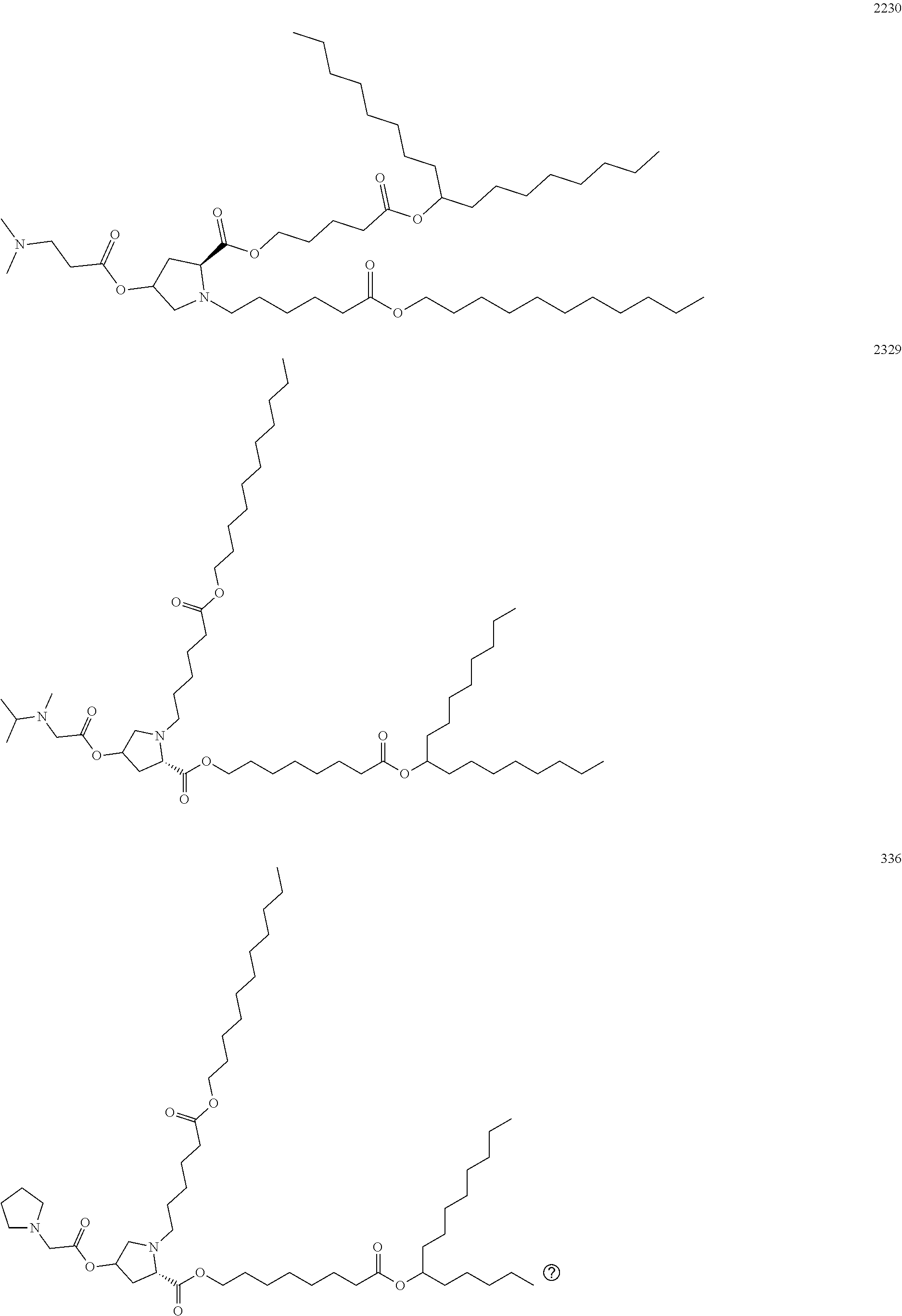

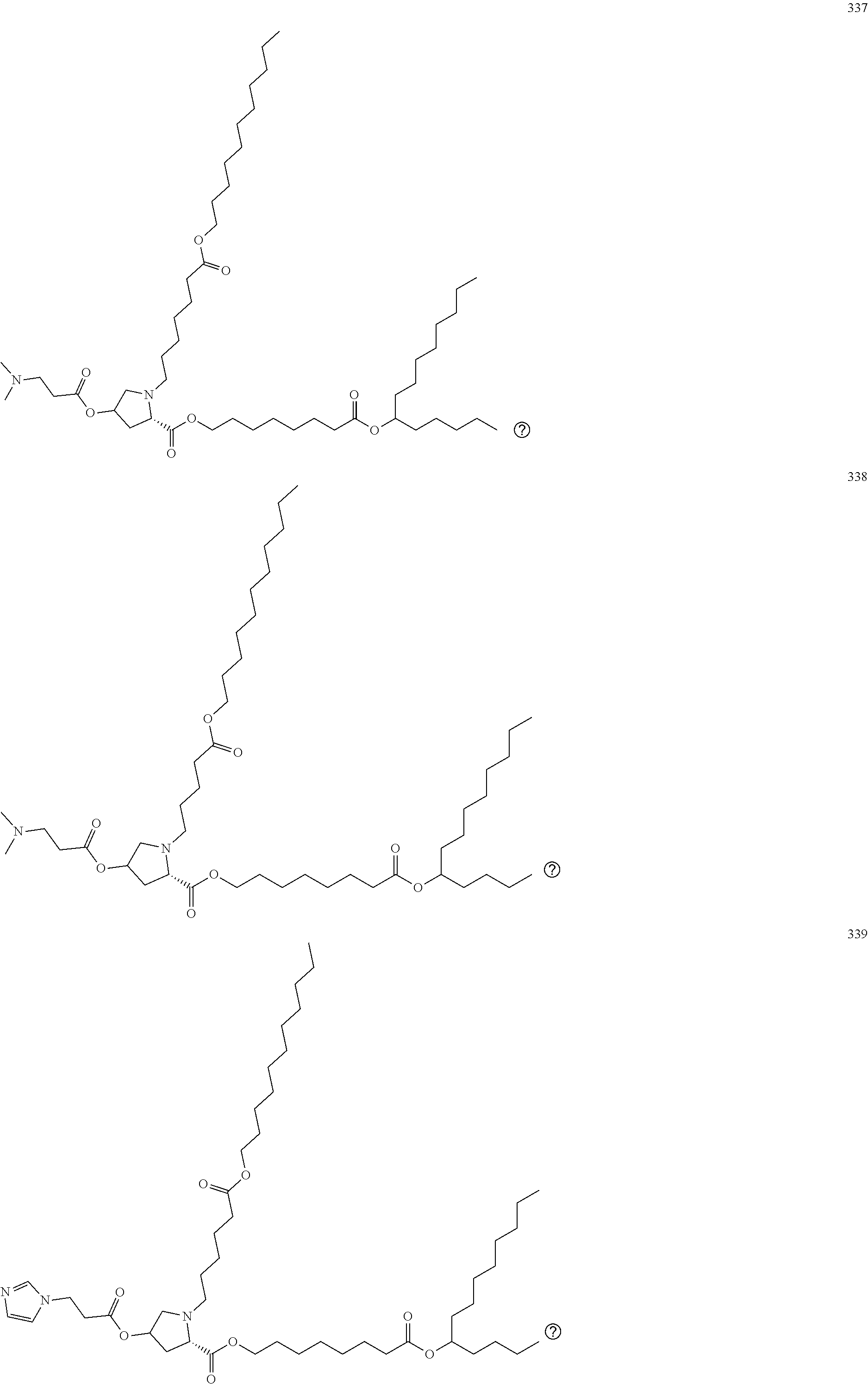

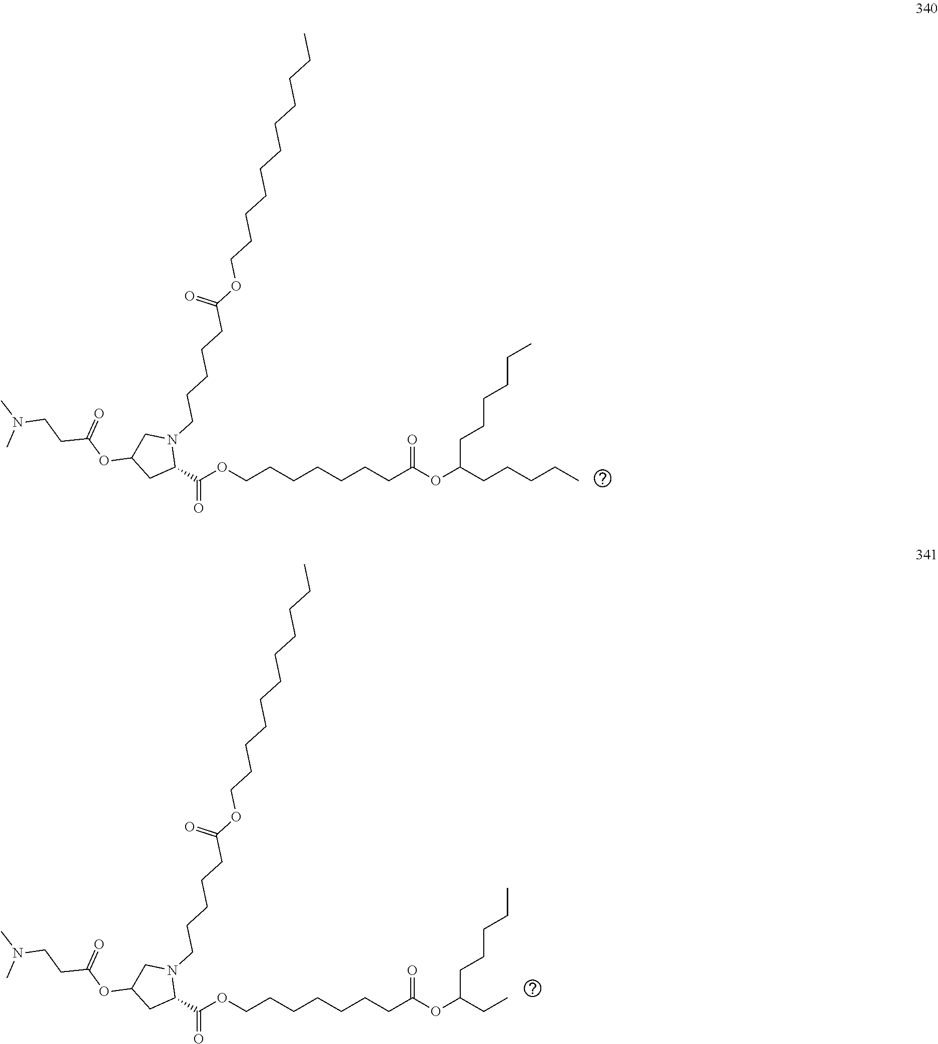

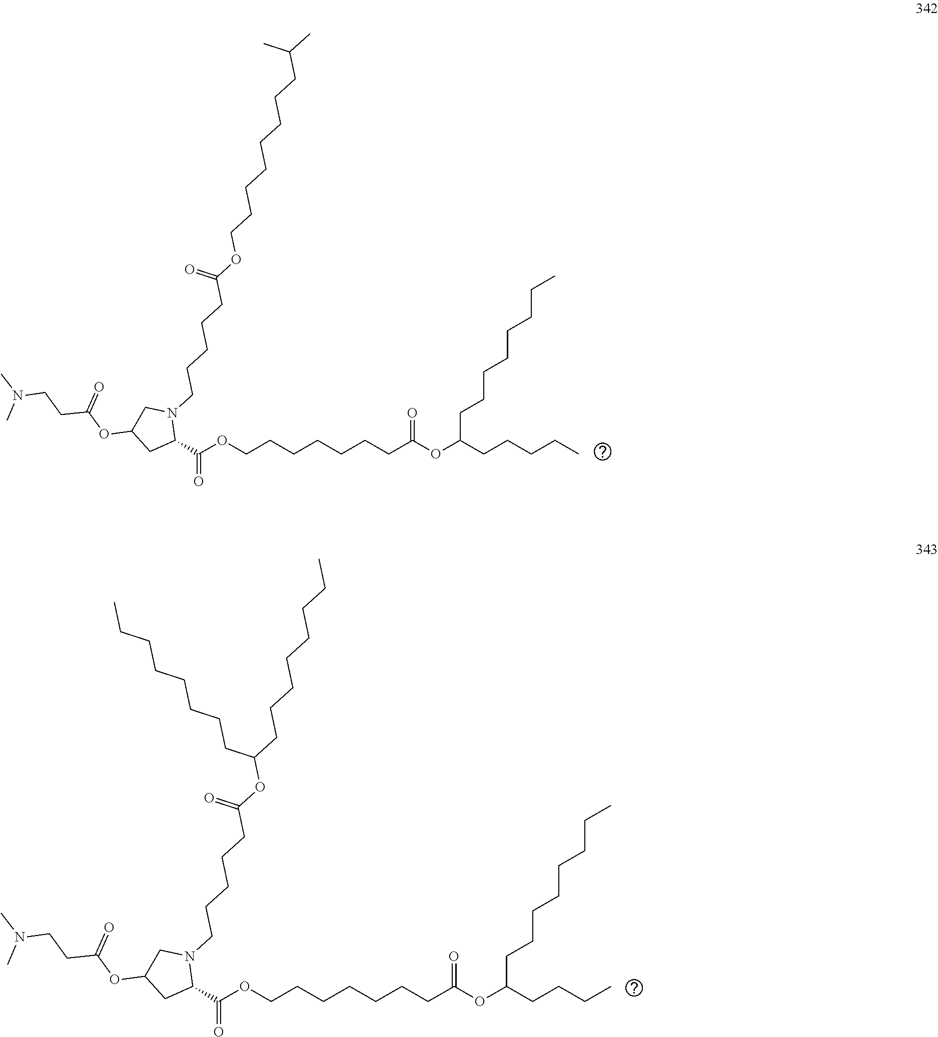

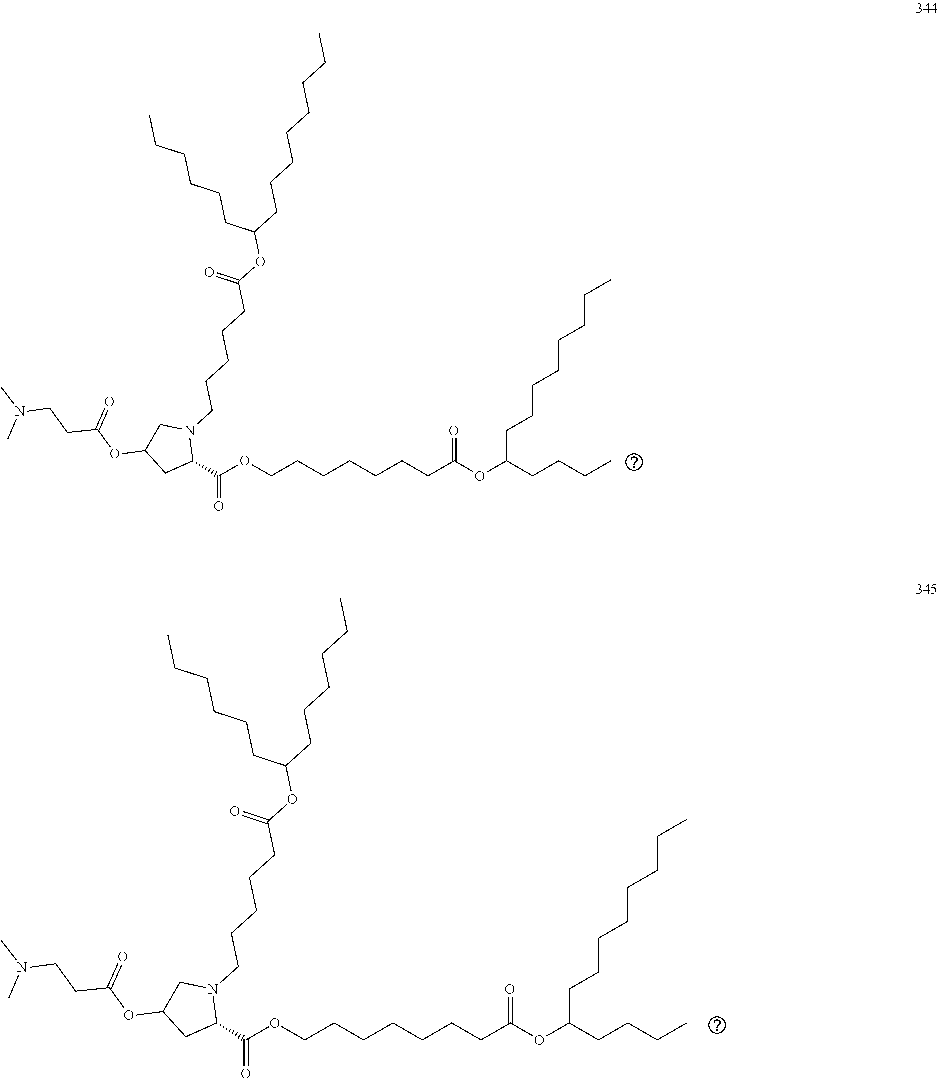

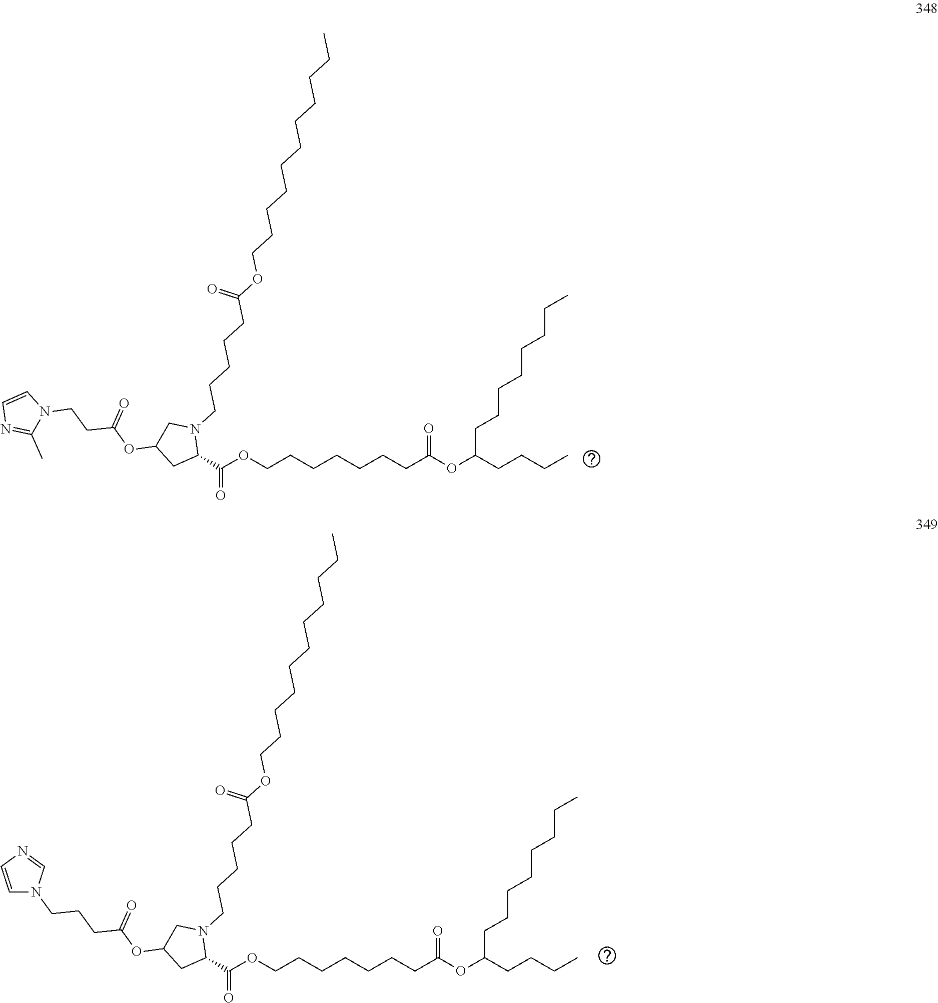 [SMILES: CCCCCCCCCCCOC(=O)CCCCCN1CC(OC(=O)CCn2ccnc2C)C[C@H]1C(=O)OCCCCCCCC(=O)OC(CCCC)CCCCCCCC ; CCCCCCCCCCCOC(=O)CCCCCN1CC(OC(=O)CCCn2ccnc2)C[C@H]1C(=O)OCCCCCCCC(=O)OC(CCCC)CCCCCCCC]

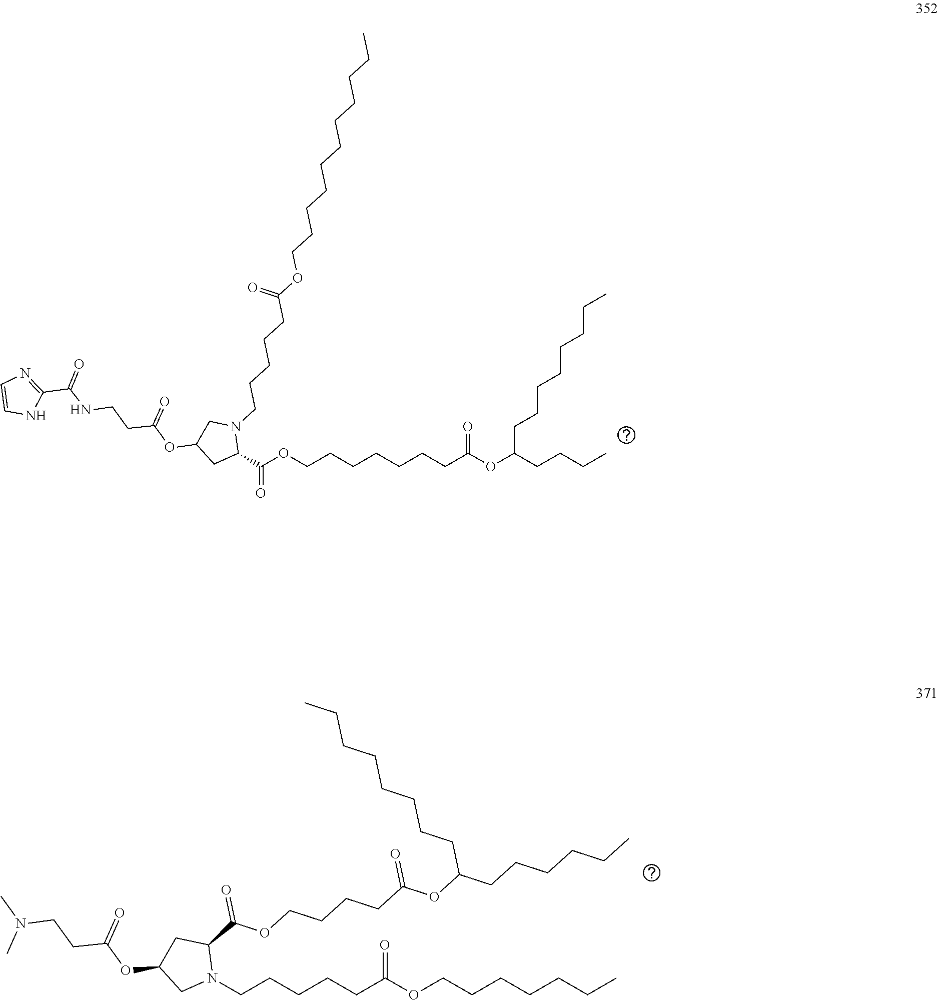

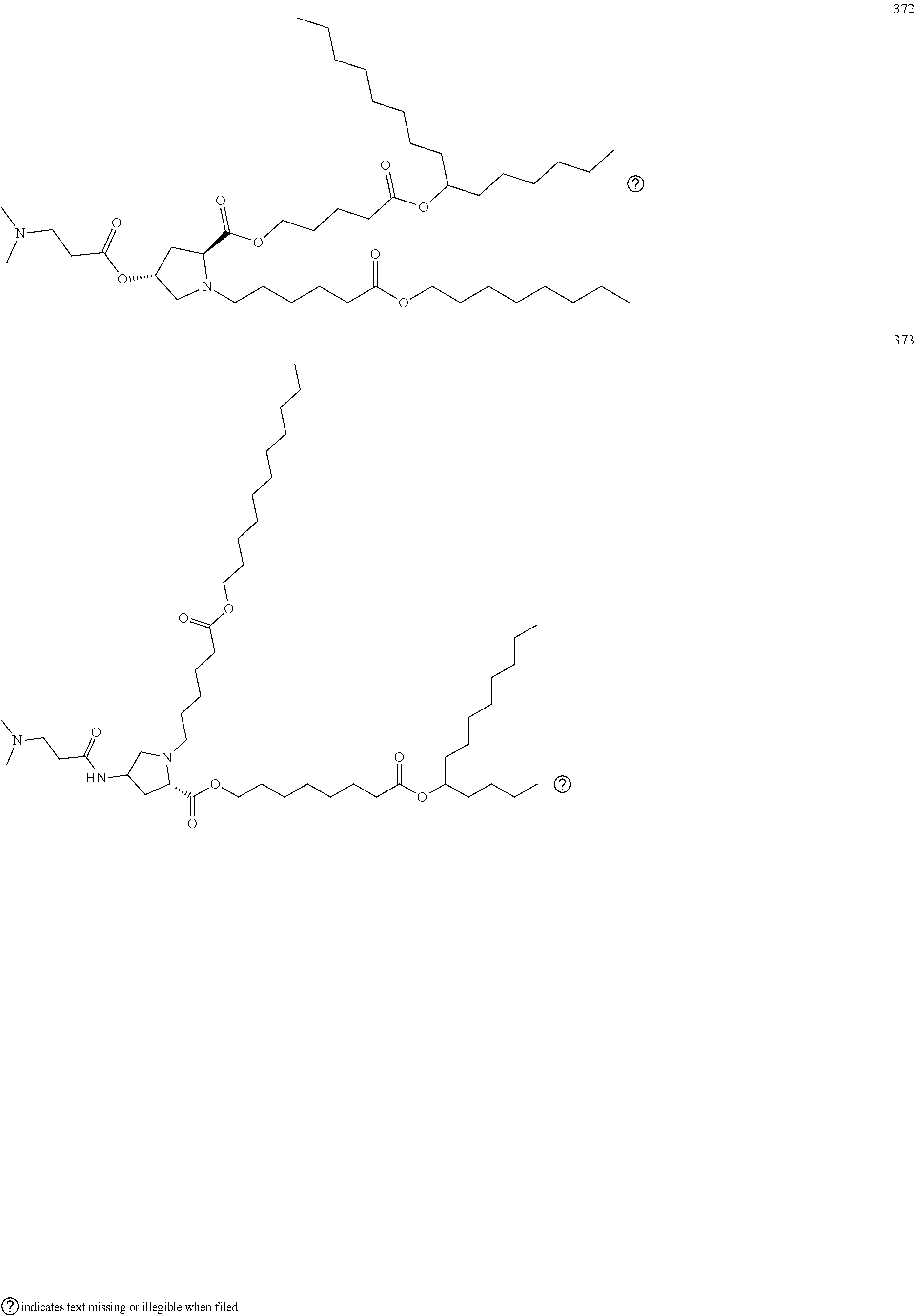

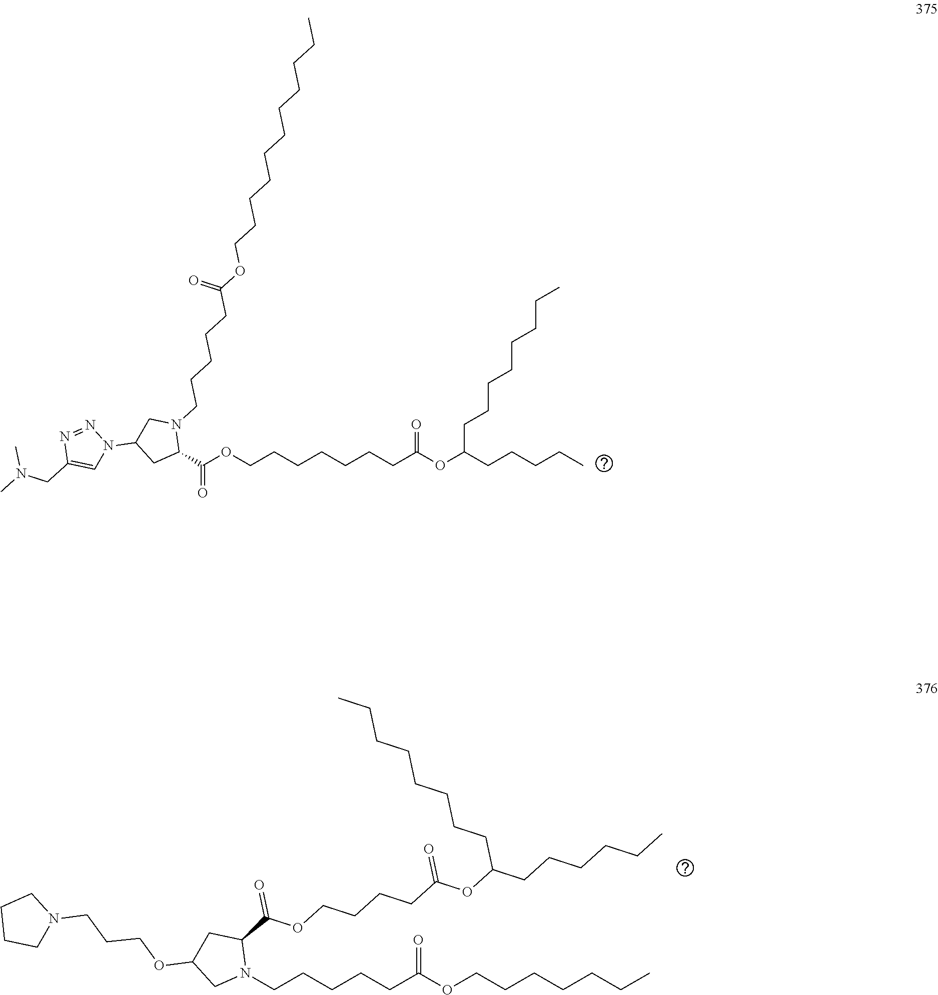 [SMILES: CCCCCCCCCCCOC(=O)CCCCCN1CC(n2cc(CN(C)C)nn2)C[C@H]1C(=O)OCCCCCCCC(=O)OC(CCCCC)CCCCCCCC ; CCCCCCCCC(CCCCCC)OC(=O)CCCCOC(=O)[C@@H]1CC(OCCCN2CCCC2)CN1CCCCCC(=O)OCCCCCCC]

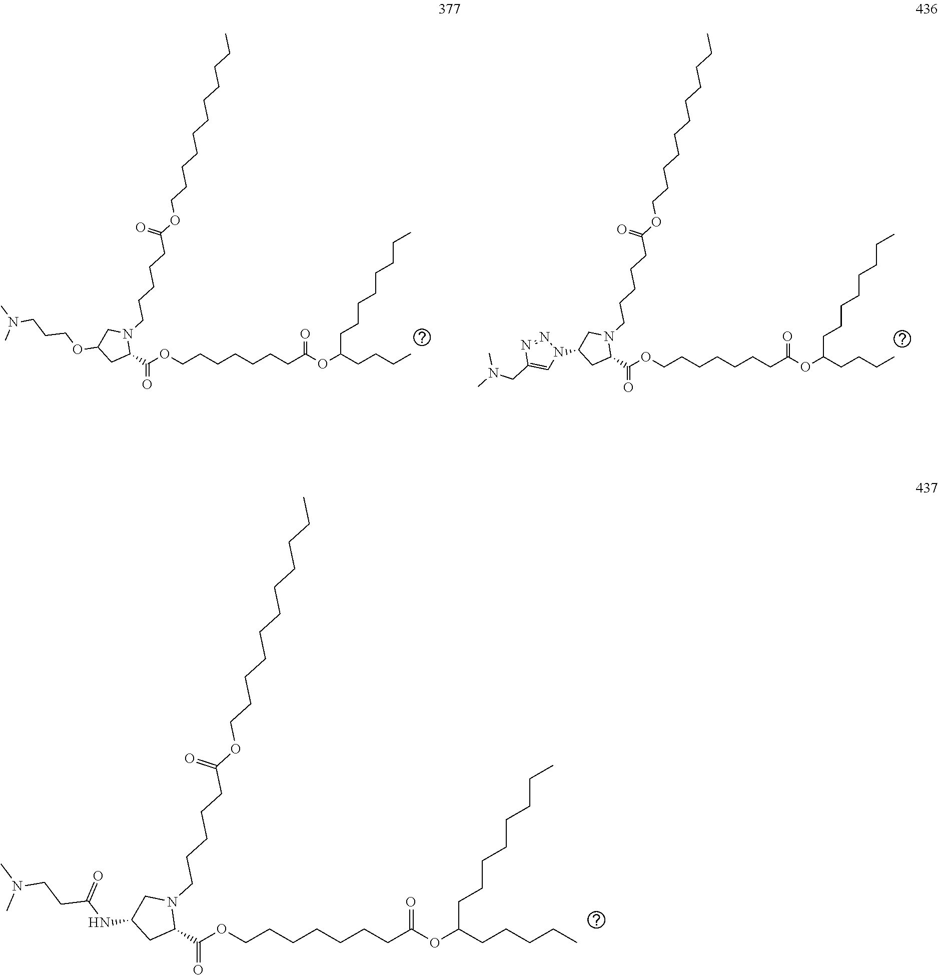 [SMILES: CCCCCCCCCCCOC(=O)CCCCCN1CC(OCCCN(C)C)C[C@H]1C(=O)OCCCCCCCC(=O)OC(CCCC)CCCCCCCC ; CCCCCCCCCCCOC(=O)CCCCCN1C[C@@H](n2cc(CN(C)C)nn2)C[C@H]1C(=O)OCCCCCCCC(=O)OC(CCCC)CCCCCCCC ; CCCCCCCCCCCOC(=O)CCCCCN1C[C@@H](NC(=O)CCN(C)C)C[C@H]1C(=O)OCCCCCCCC(=O)OC(CCCCC)CCCCCCCC]

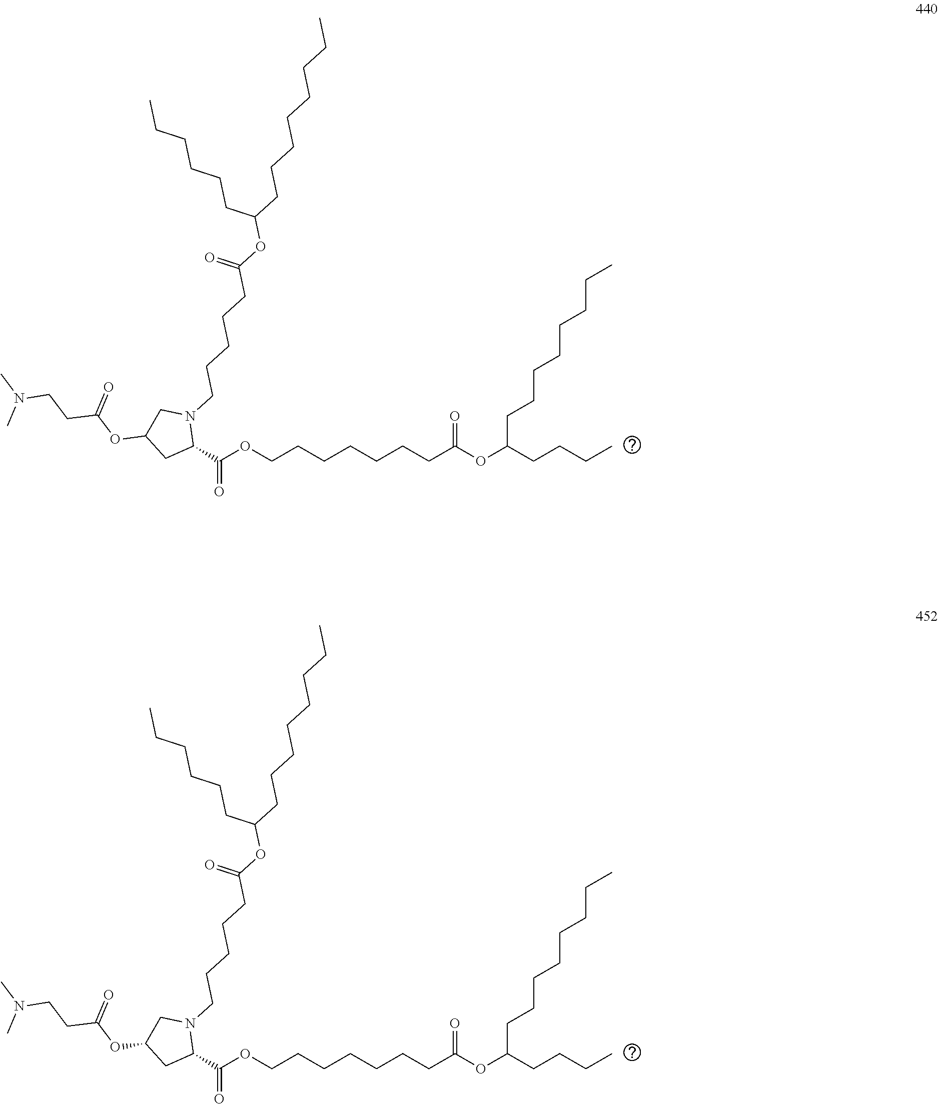

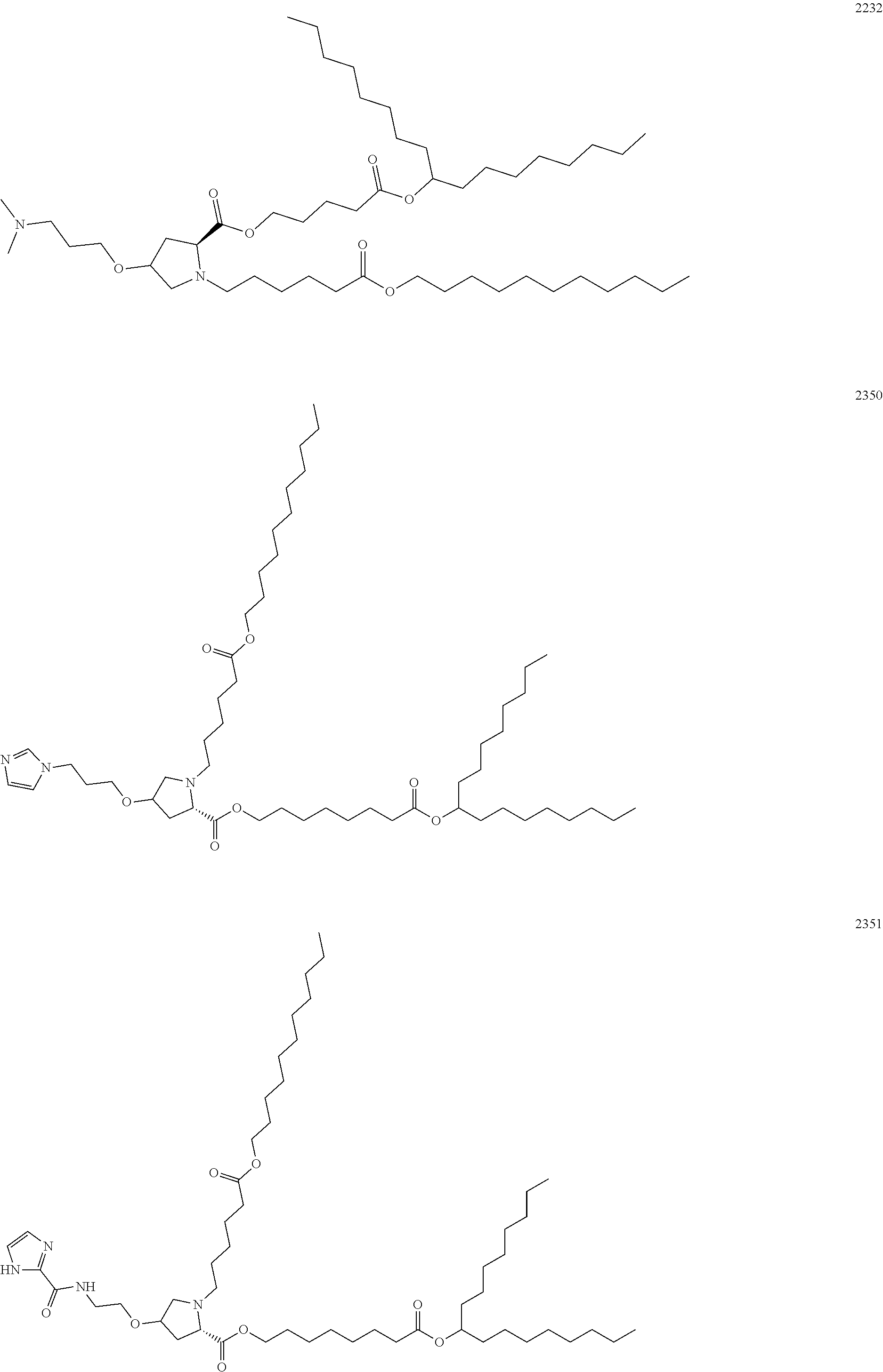 [SMILES: CCCCCCCCCCCOC(=O)CCCCCN1CC(OCCCN(C)C)C[C@H]1C(=O)OCCCCC(=O)OC(CCCCCCCC)CCCCCCCC ; CCCCCCCCCCCOC(=O)CCCCCN1CC(OCCCn2ccnc2)C[C@H]1C(=O)OCCCCCCCC(=O)OC(CCCCCCCC)CCCCCCCC ; CCCCCCCCCCCOC(=O)CCCCCN1CC(OCCNC(=O)c2ncc[nH]2)C[C@H]1C(=O)OCCCCCCCC(=O)OC(CCCCCCCC)CCCCCCCC]

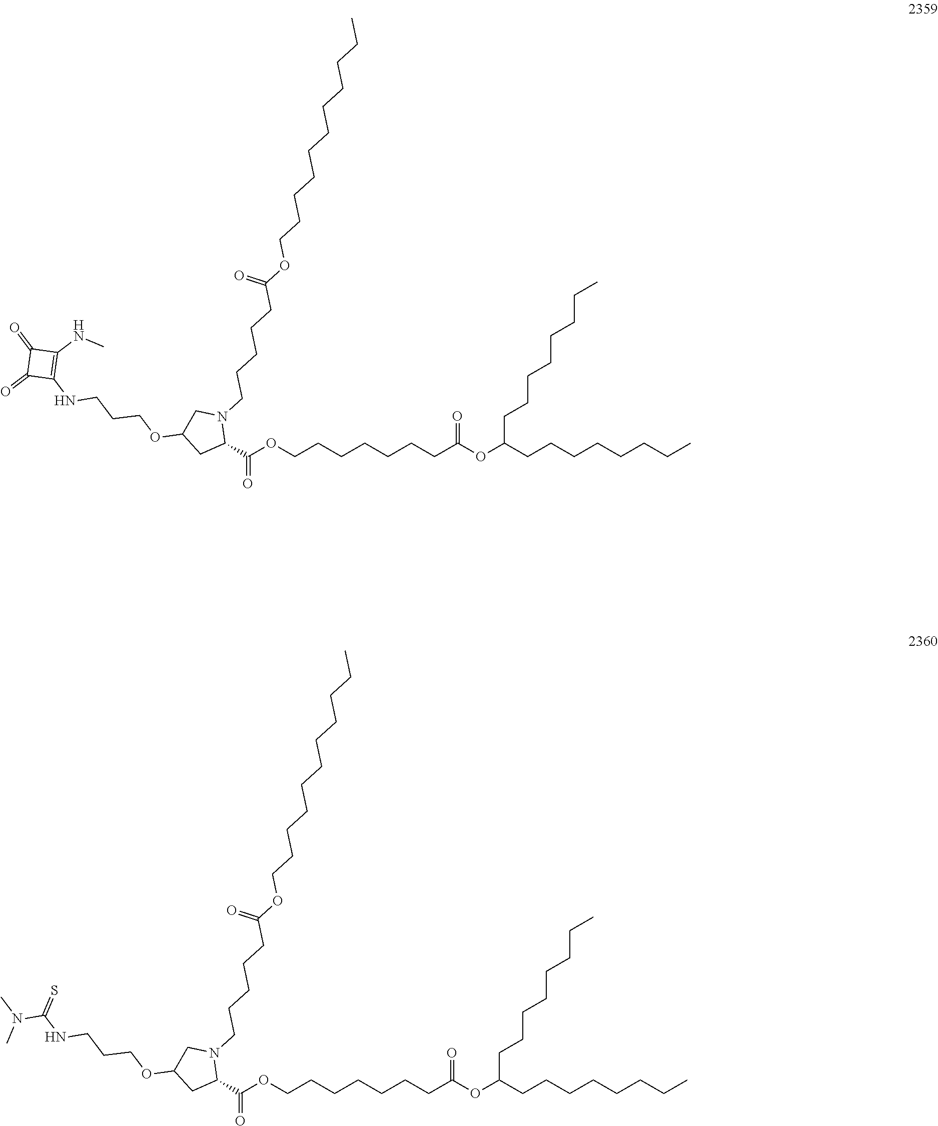

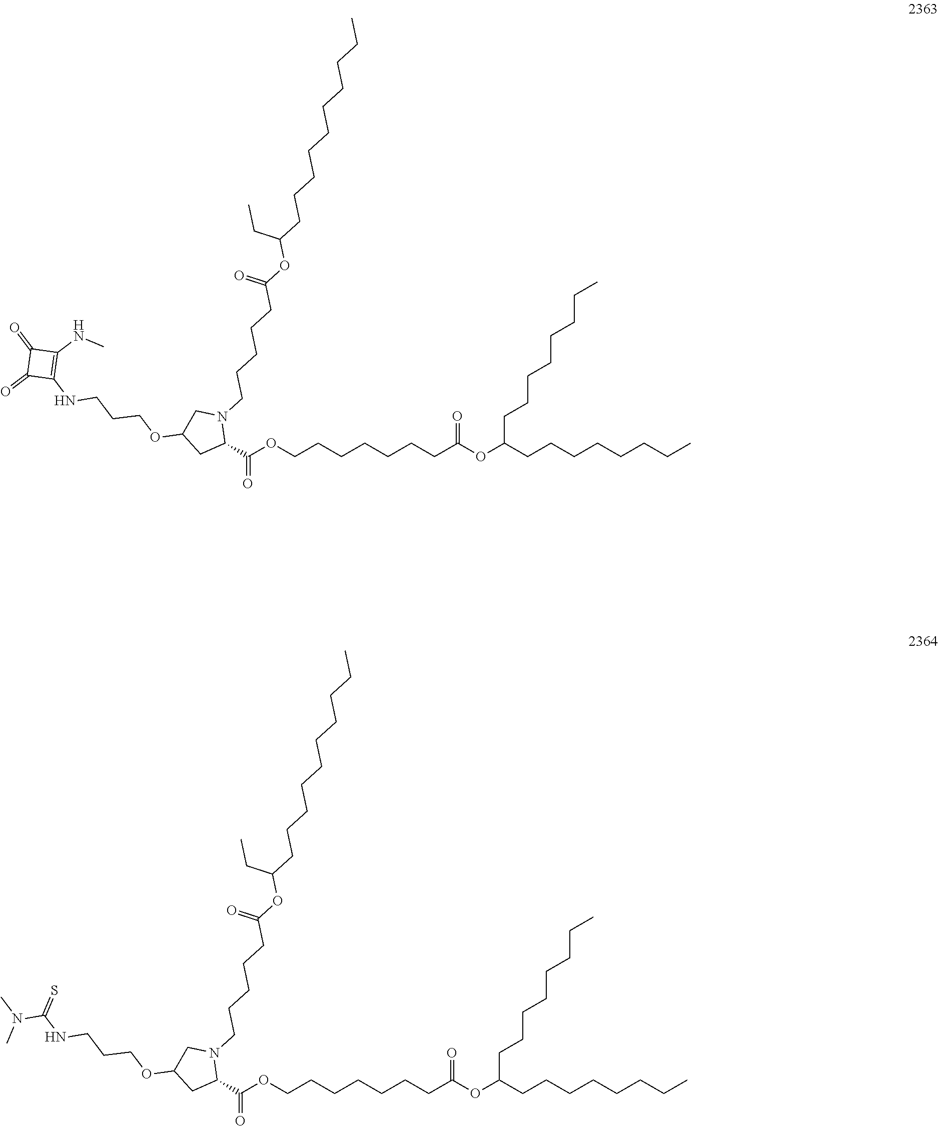

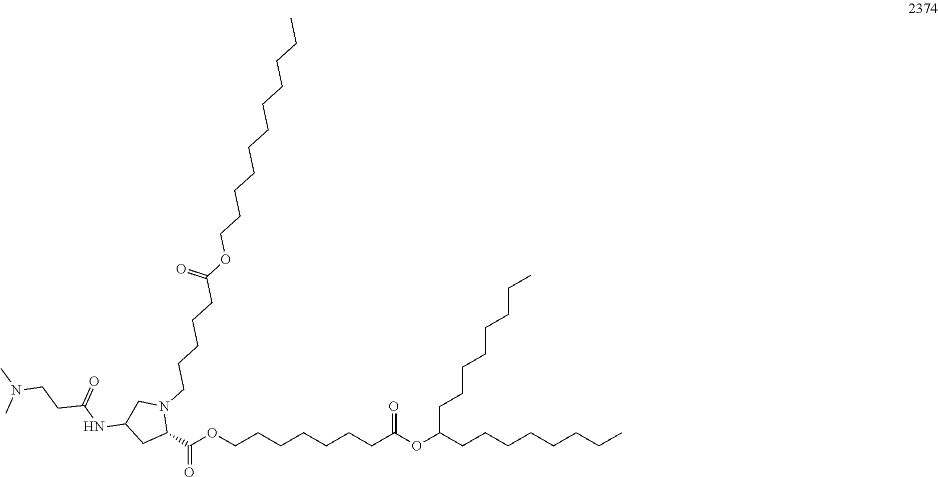 [SMILES: CCCCCCCCCCCOC(=O)CCCCCN1CC(NC(=O)CCN(C)C)C[C@H]1C(=O)OCCCCCCCC(=O)OC(CCCCCCCC)CCCCCCCC]

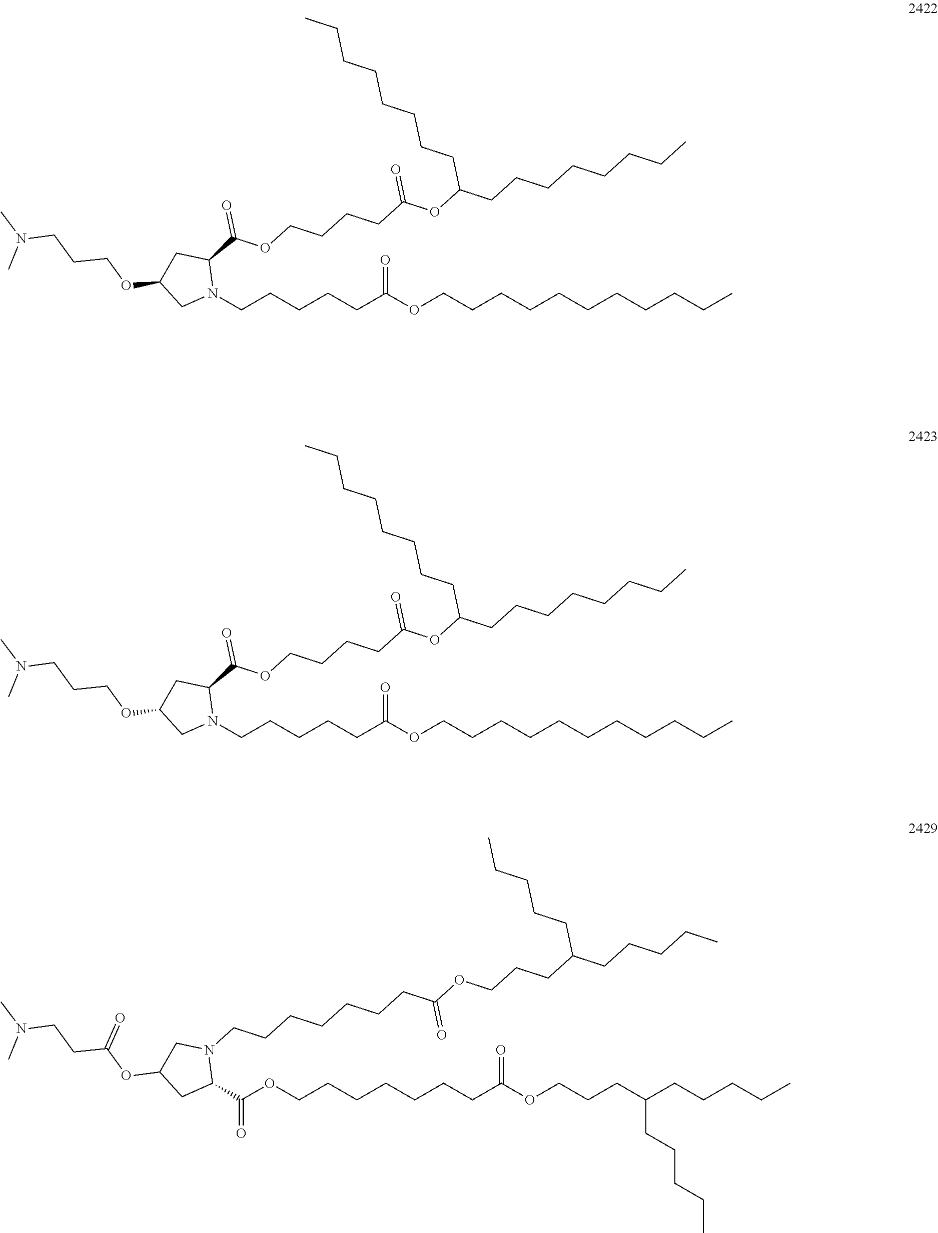

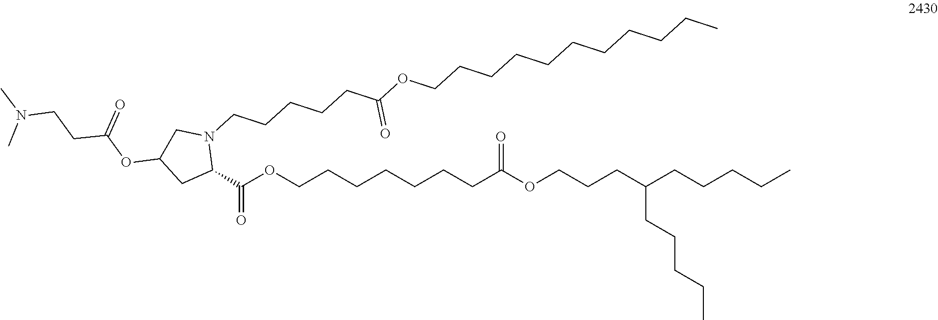 [SMILES: CCCCCCCCCCCOC(=O)CCCCCN1CC(OC(=O)CCN(C)C)C[C@H]1C(=O)OCCCCCCCC(=O)OCCCC(CCCCC)CCCCC]

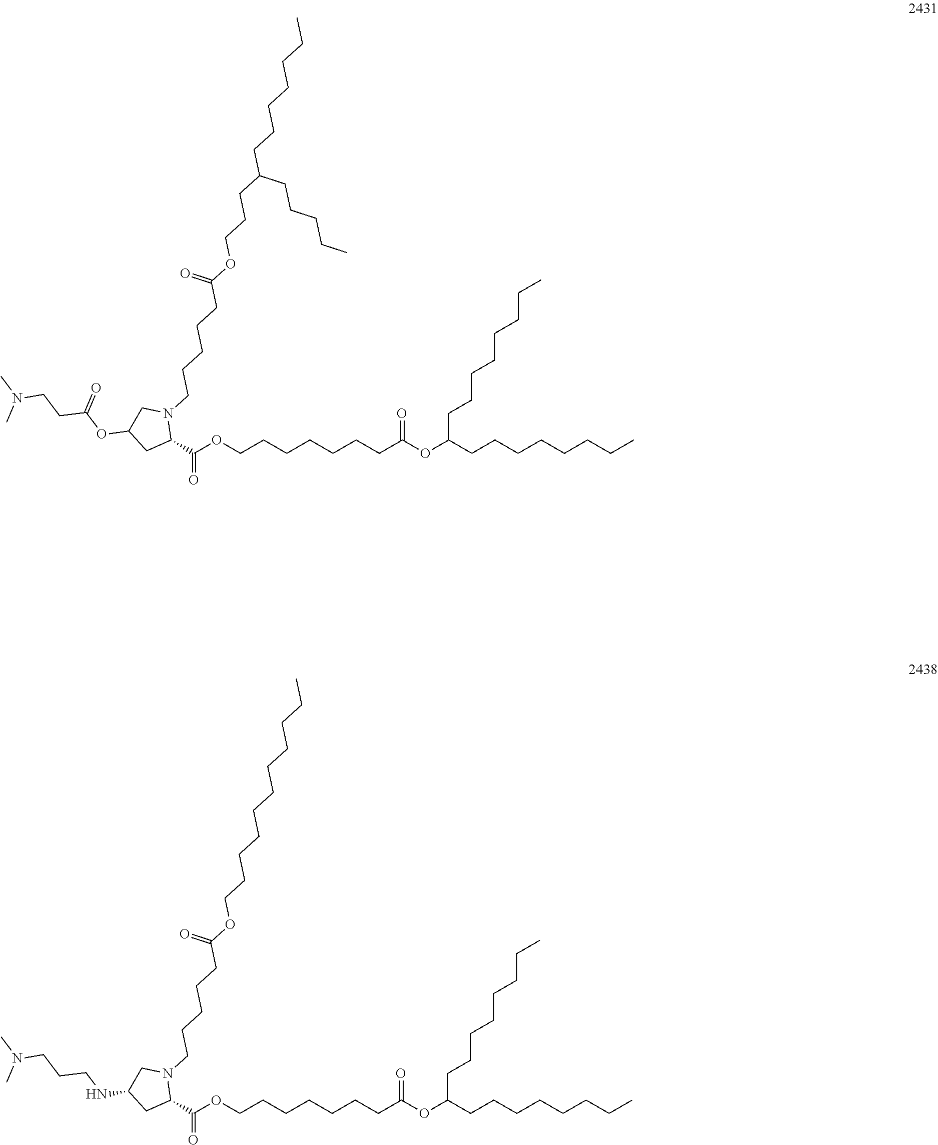

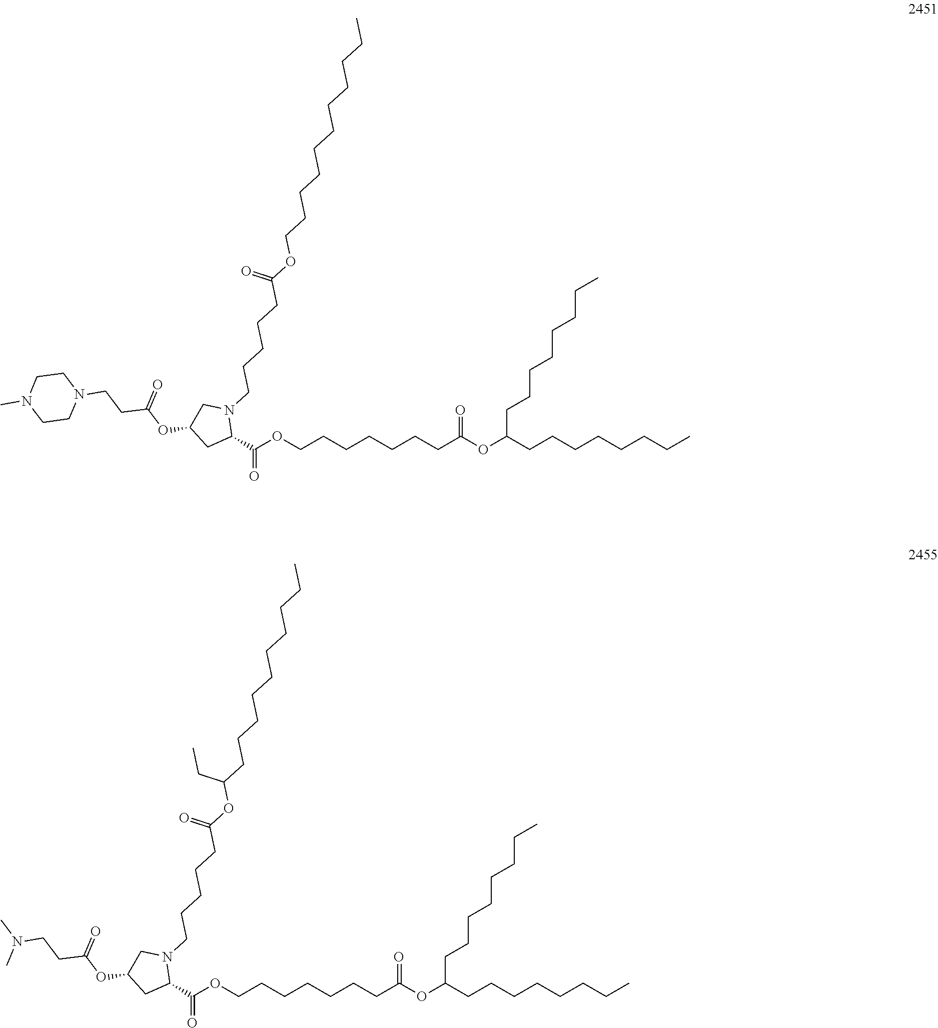

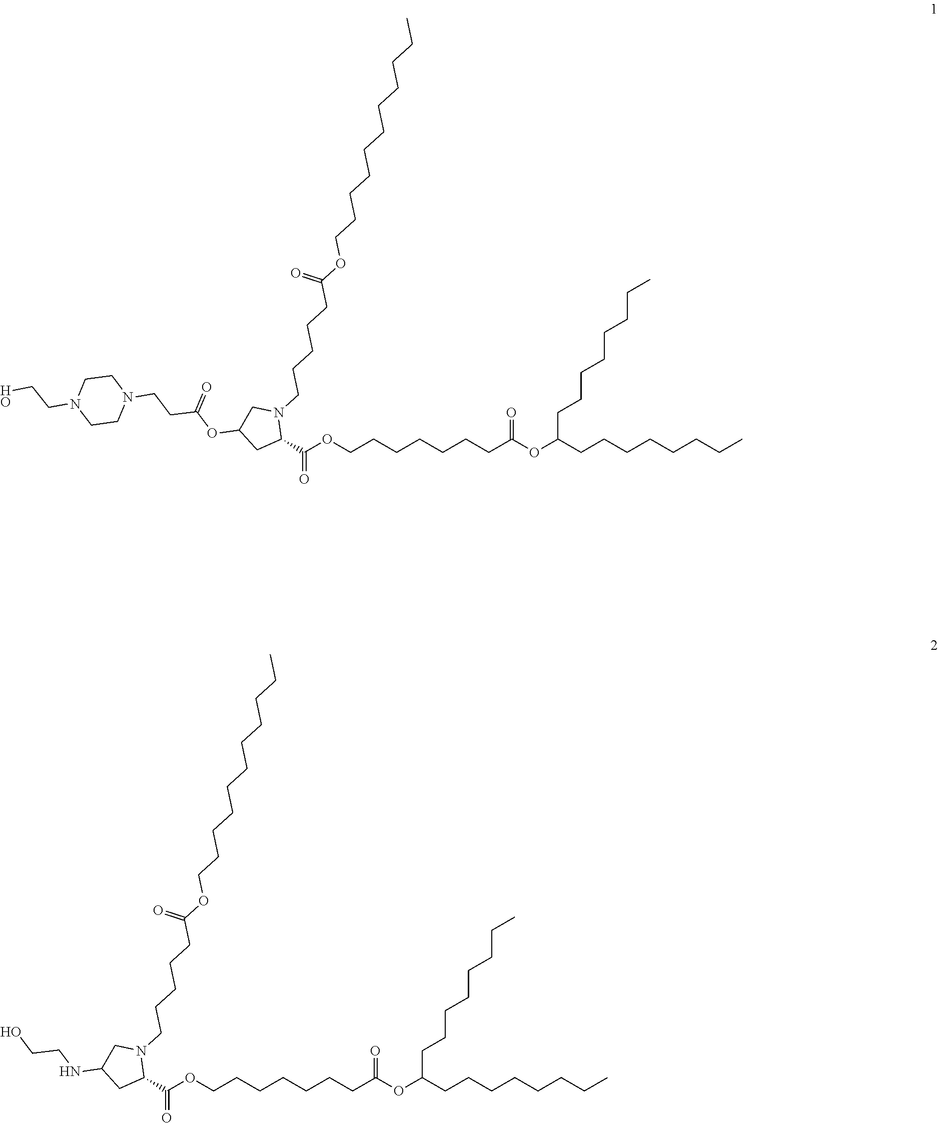 [SMILES: CCCCCCCCCCCOC(=O)CCCCCN1CC(OC(=O)CCN2CCN(CCO)CC2)C[C@H]1C(=O)OCCCCCCCC(=O)OC(CCCCCCCC)CCCCCCCC ; CCCCCCCCCCCOC(=O)CCCCCN1CC(NCCO)C[C@H]1C(=O)OCCCCCCCC(=O)OC(CCCCCCCC)CCCCCCCC]

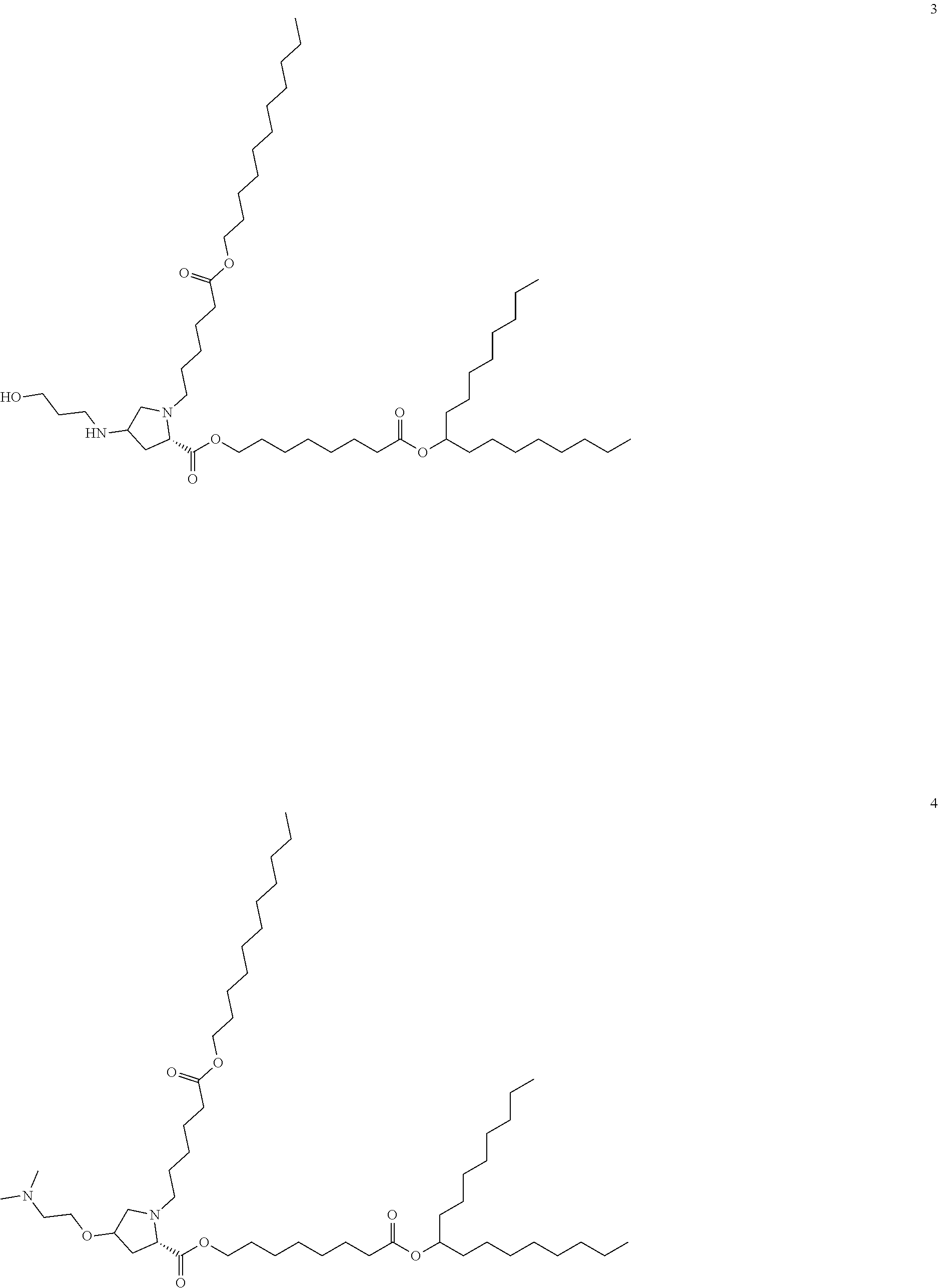

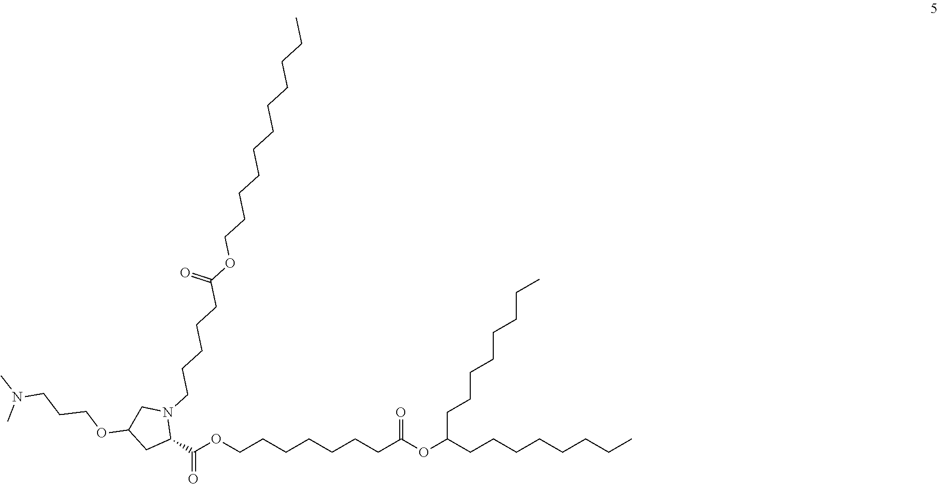 [SMILES: CCCCCCCCCCCOC(=O)CCCCCN1CC(OCCCN(C)C)C[C@H]1C(=O)OCCCCCCCC(=O)OC(CCCCCCCC)CCCCCCCC]

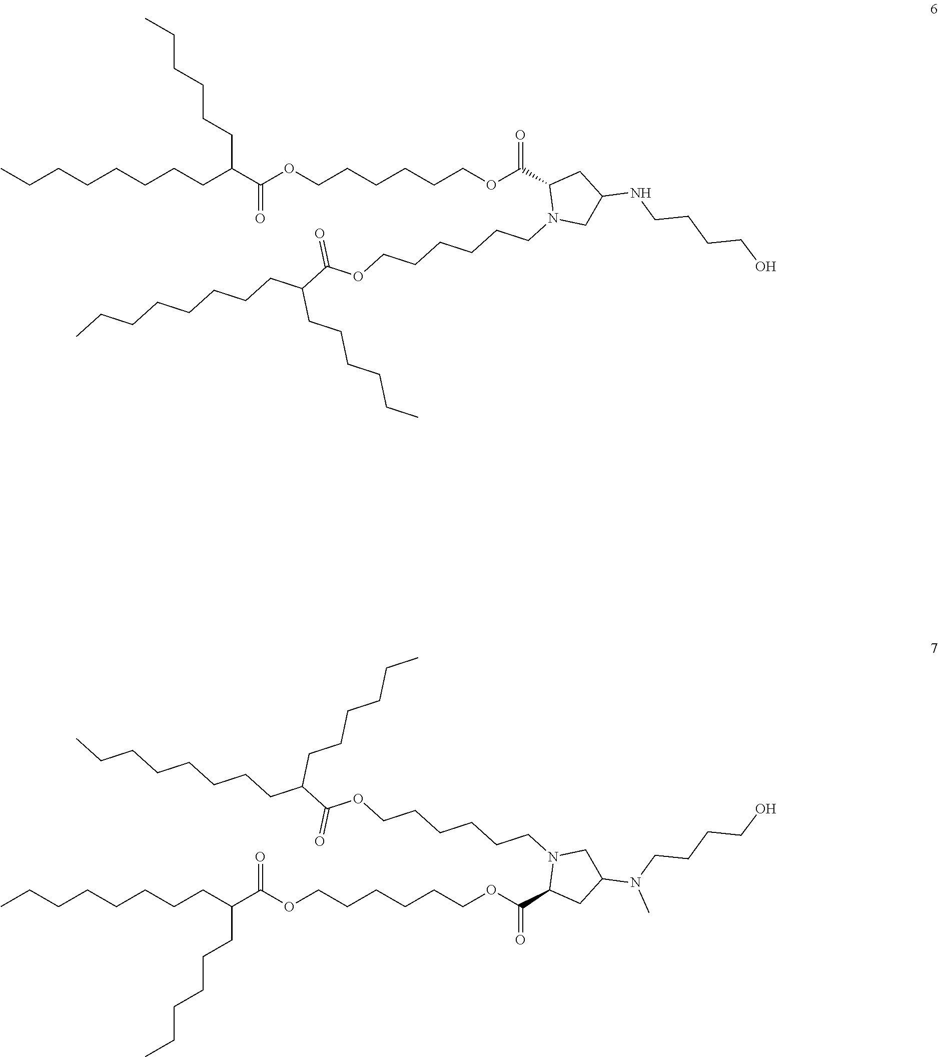 [SMILES: CCCCCCCCC(CCCCCC)C(=O)OCCCCCCOC(=O)[C@@H]1CC(NCCCCO)CN1CCCCCCOC(=O)C(CCCCCC)CCCCCCCC ; CCCCCCCCC(CCCCCC)C(=O)OCCCCCCOC(=O)[C@@H]1CC(N(C)CCCCO)CN1CCCCCCOC(=O)C(CCCCCC)CCCCCCCC]

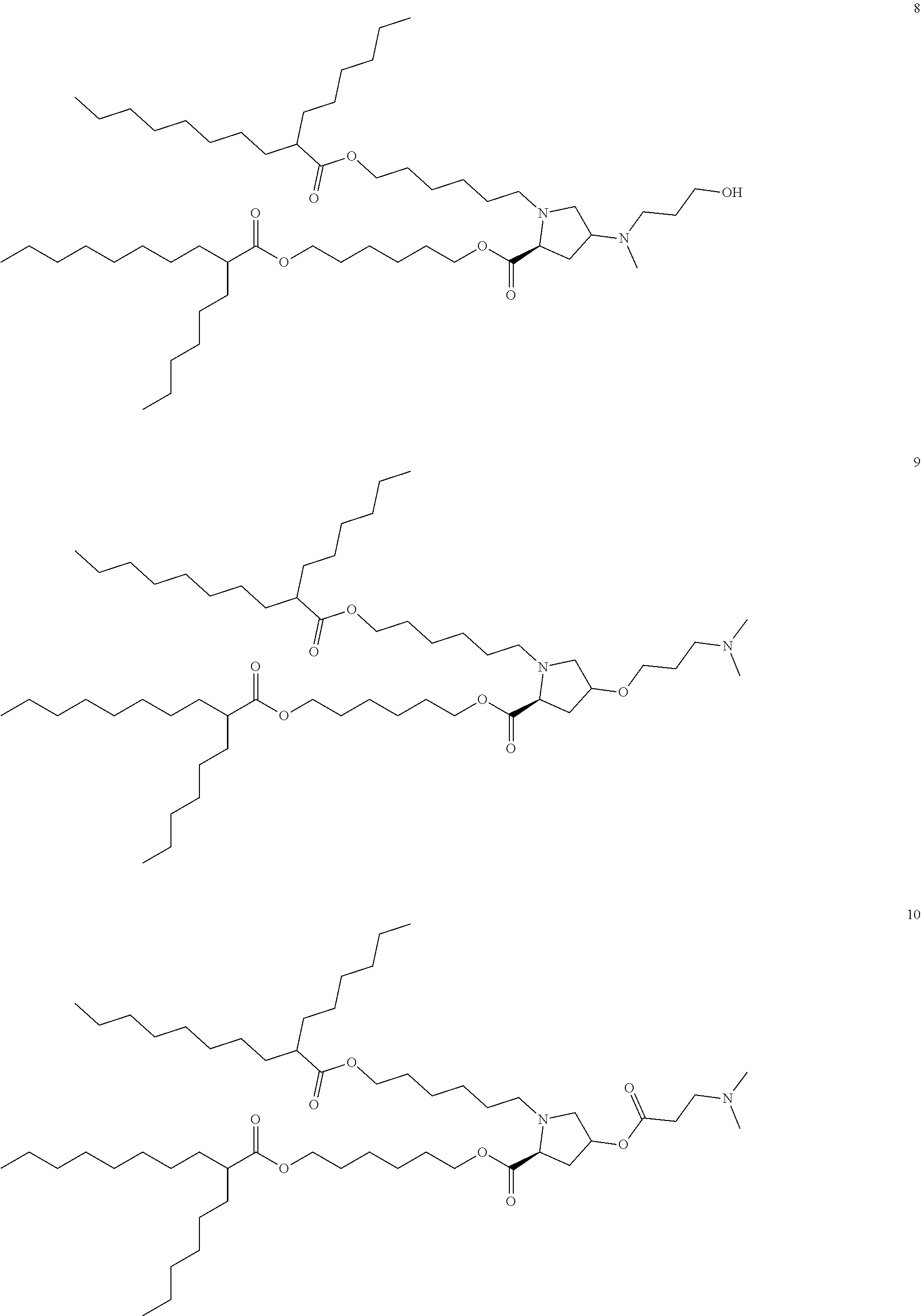

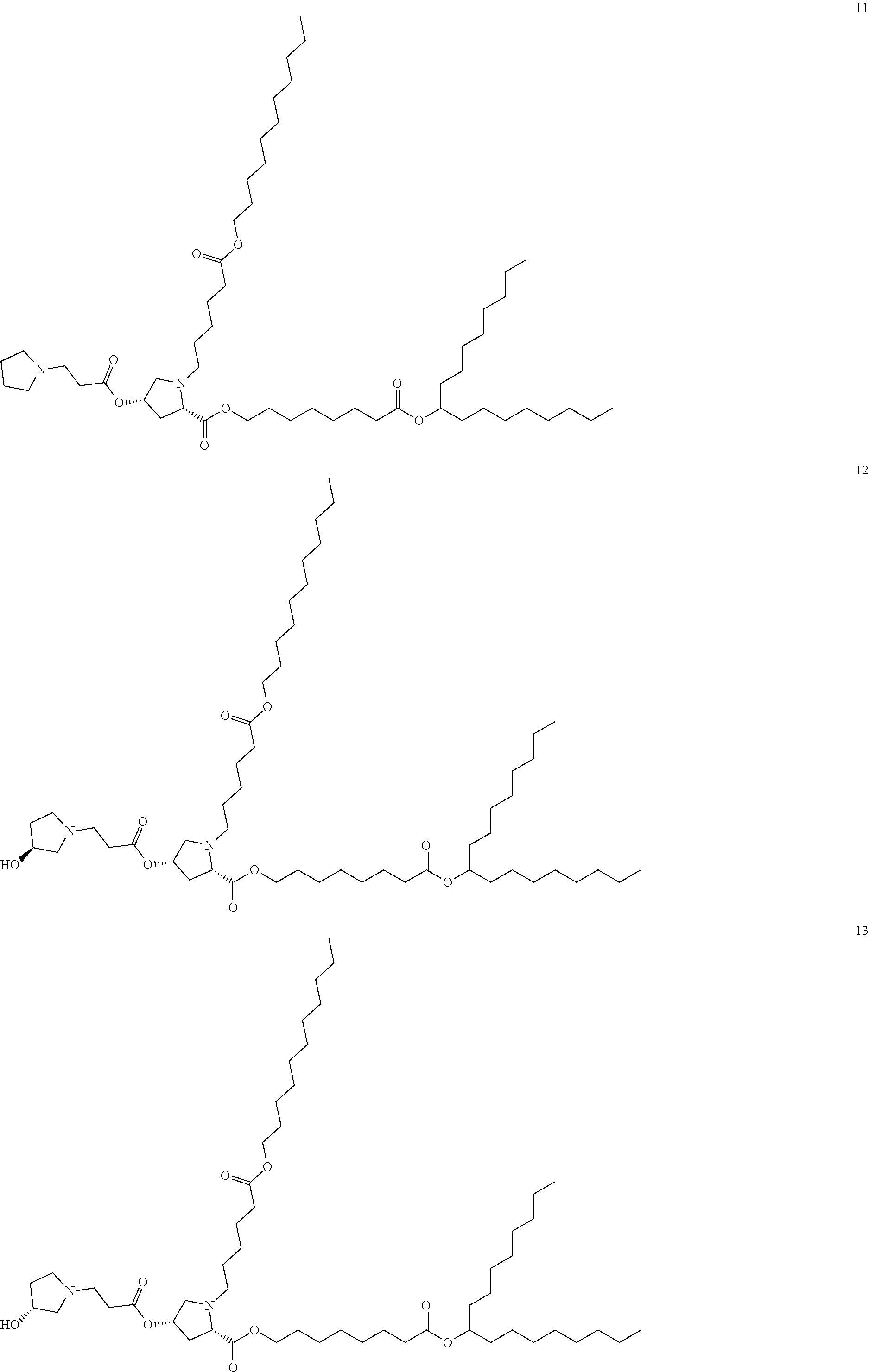 [SMILES: CCCCCCCCCCCOC(=O)CCCCCN1C[C@@H](OC(=O)CCN2CCCC2)C[C@H]1C(=O)OCCCCCCCC(=O)OC(CCCCCCCC)CCCCCCCC ; CCCCCCCCCCCOC(=O)CCCCCN1C[C@@H](OC(=O)CCN2CC[C@H](O)C2)C[C@H]1C(=O)OCCCCCCCC(=O)OC(CCCCCCCC)CCCCCCCC ; CCCCCCCCCCCOC(=O)CCCCCN1C[C@@H](OC(=O)CCN2CC[C@@H](O)C2)C[C@H]1C(=O)OCCCCCCCC(=O)OC(CCCCCCCC)CCCCCCCC]

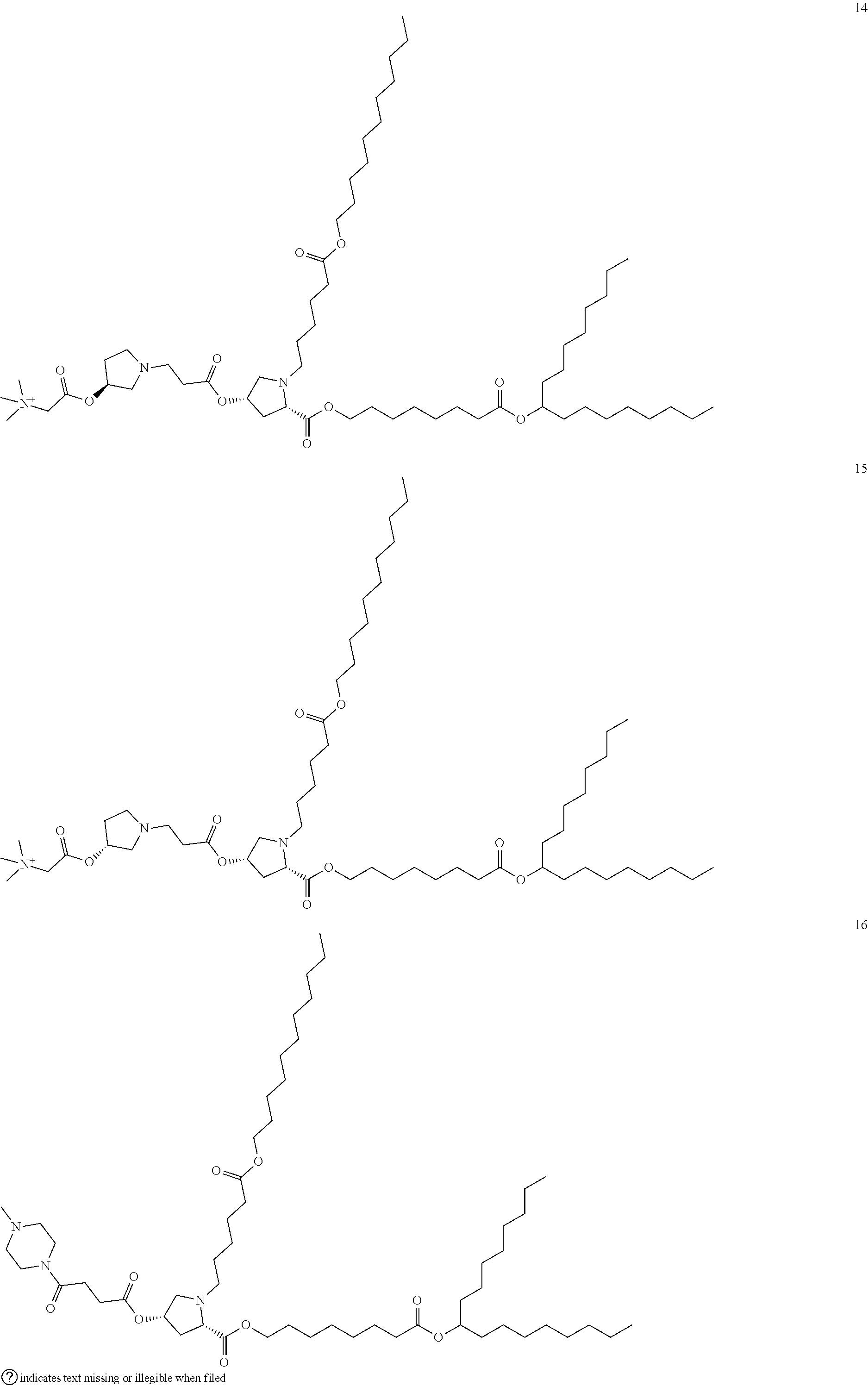

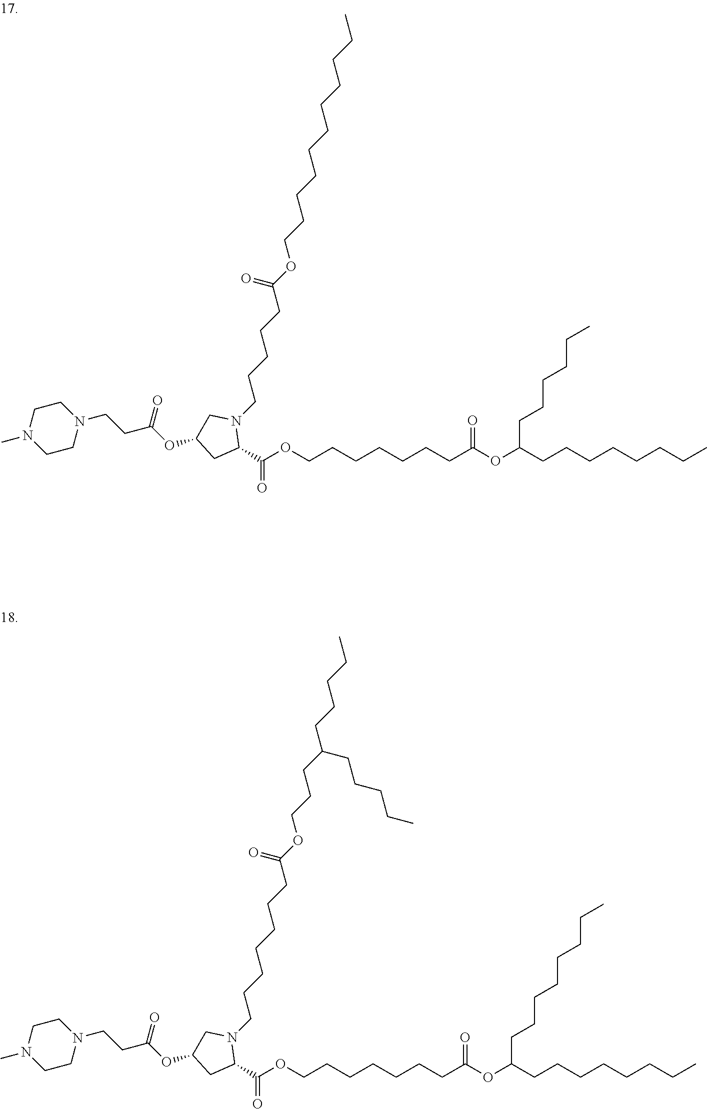

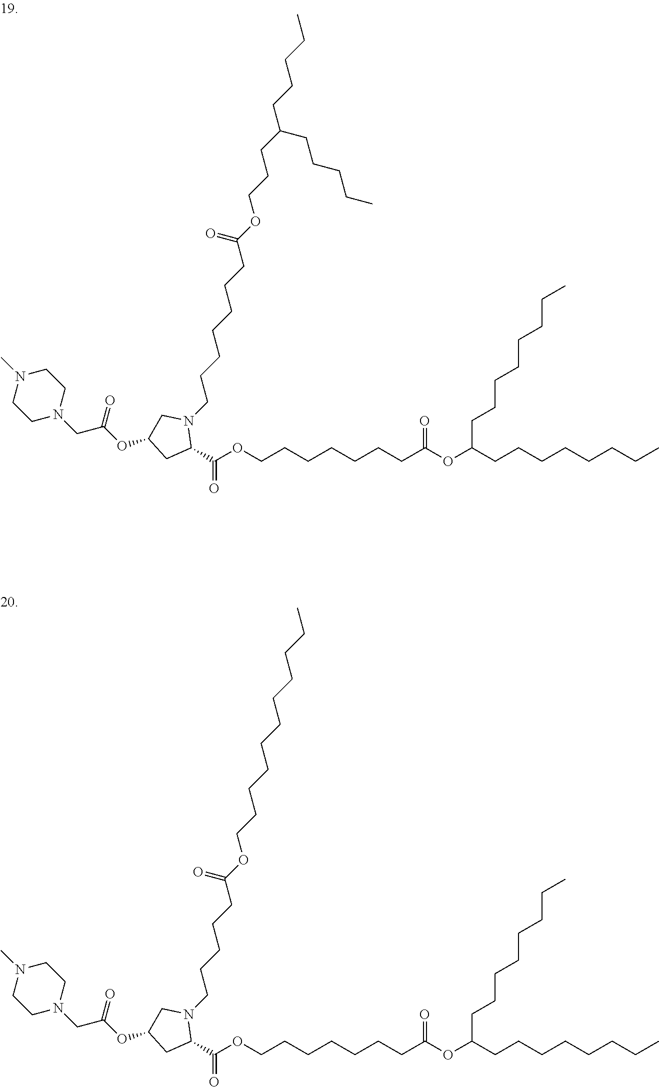

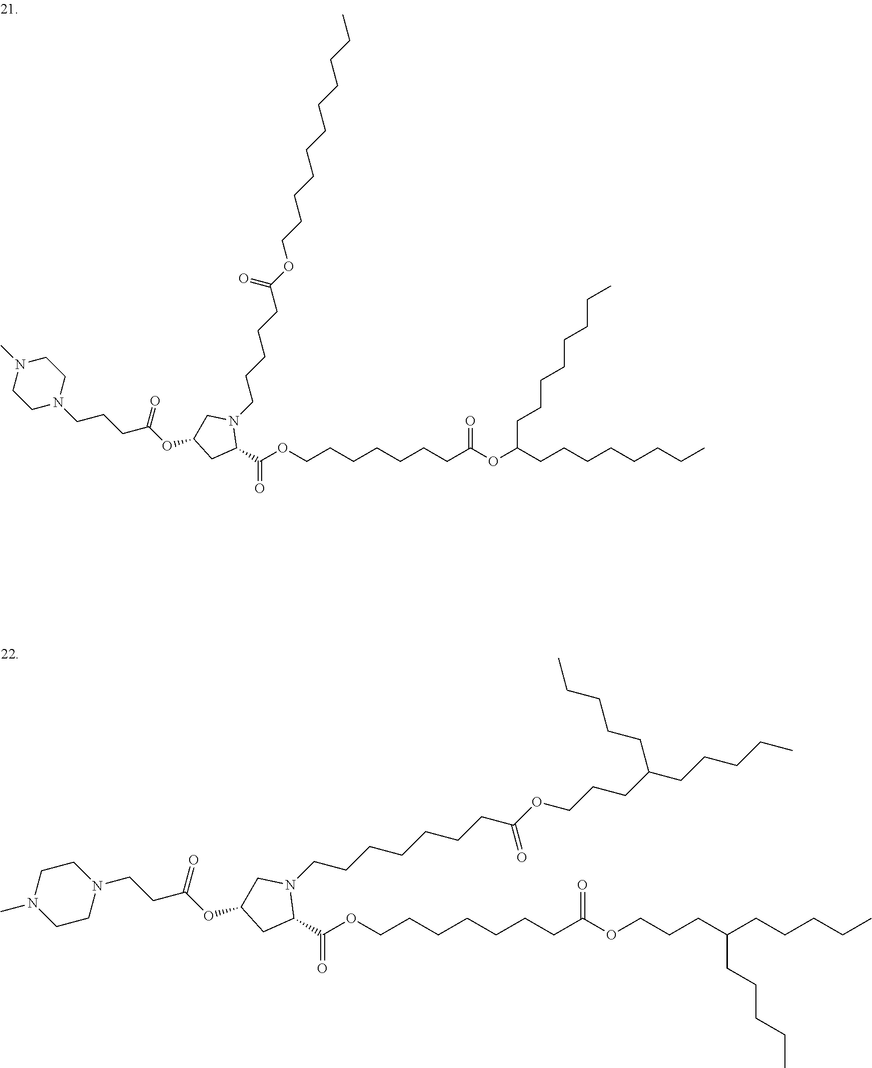

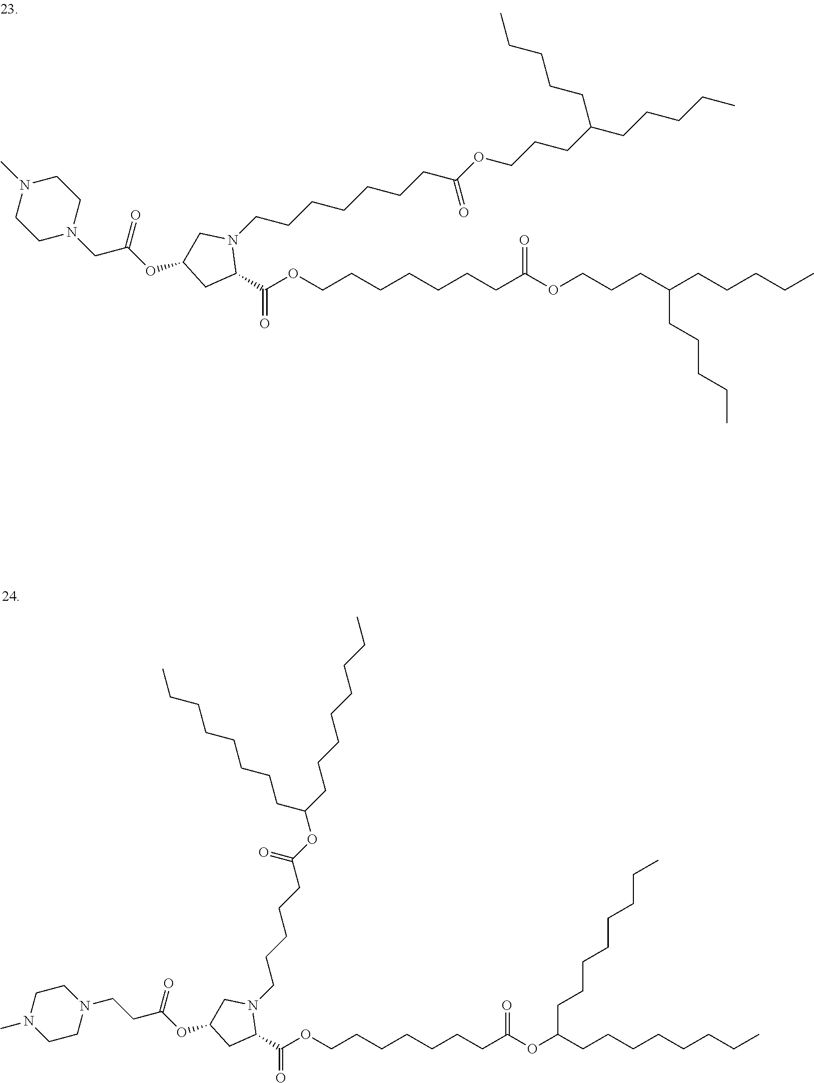

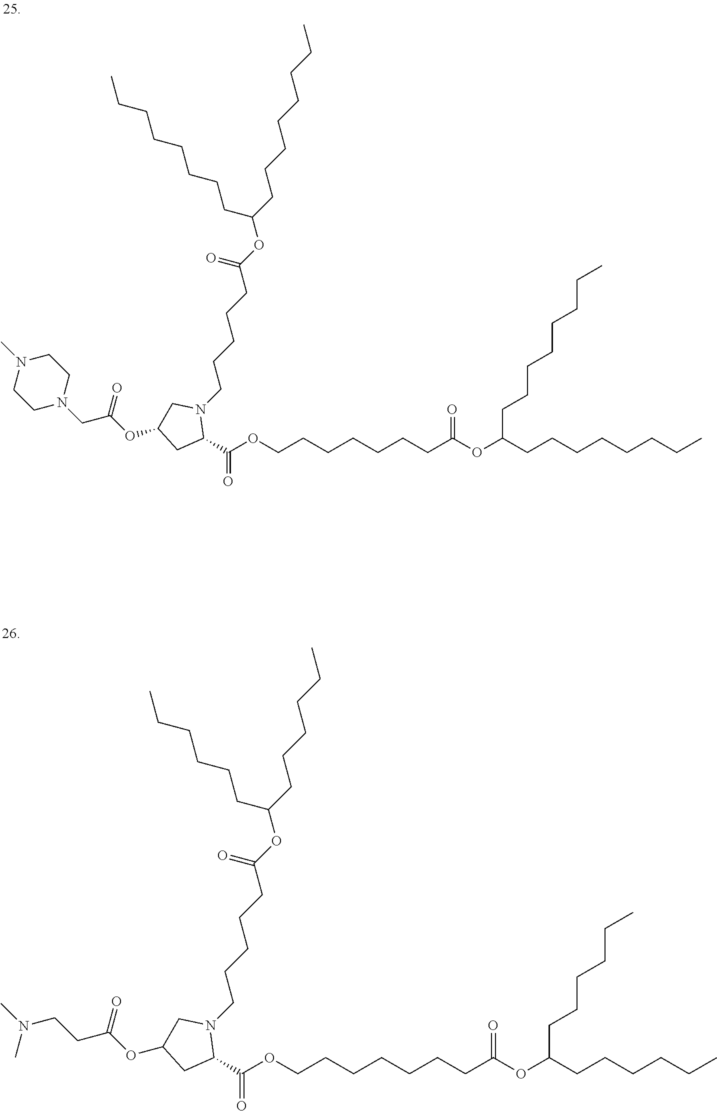

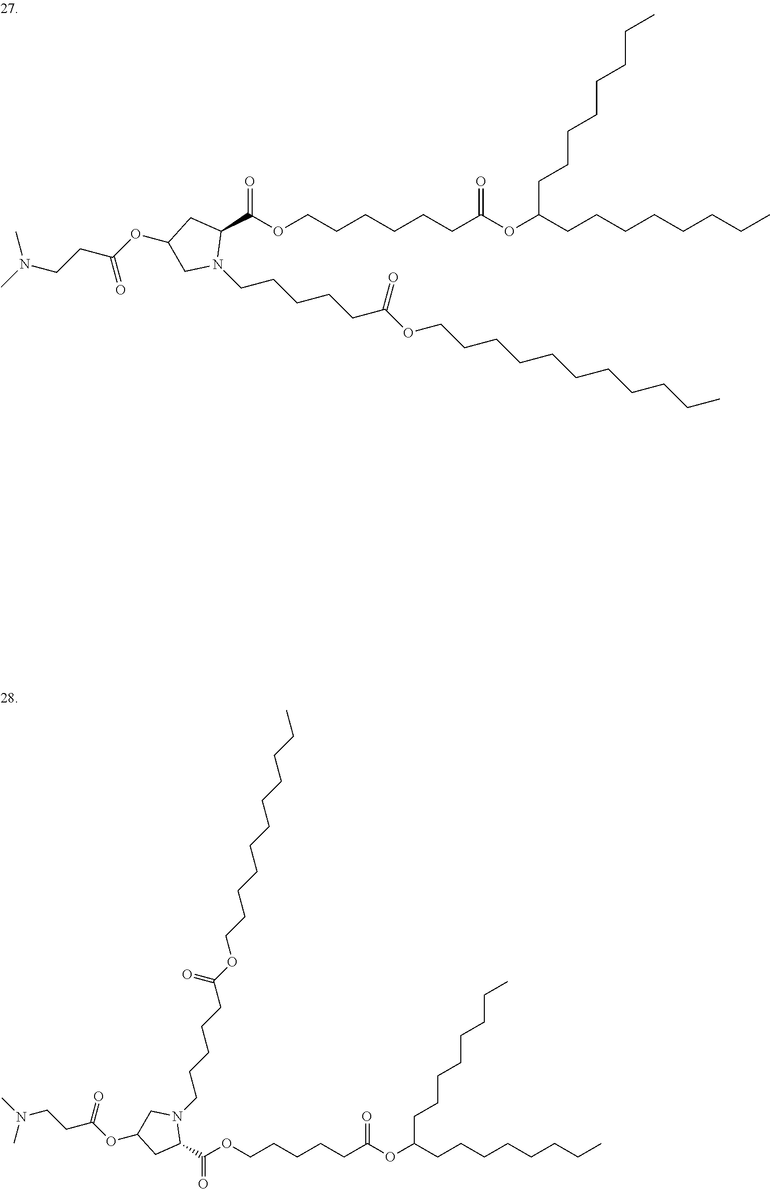

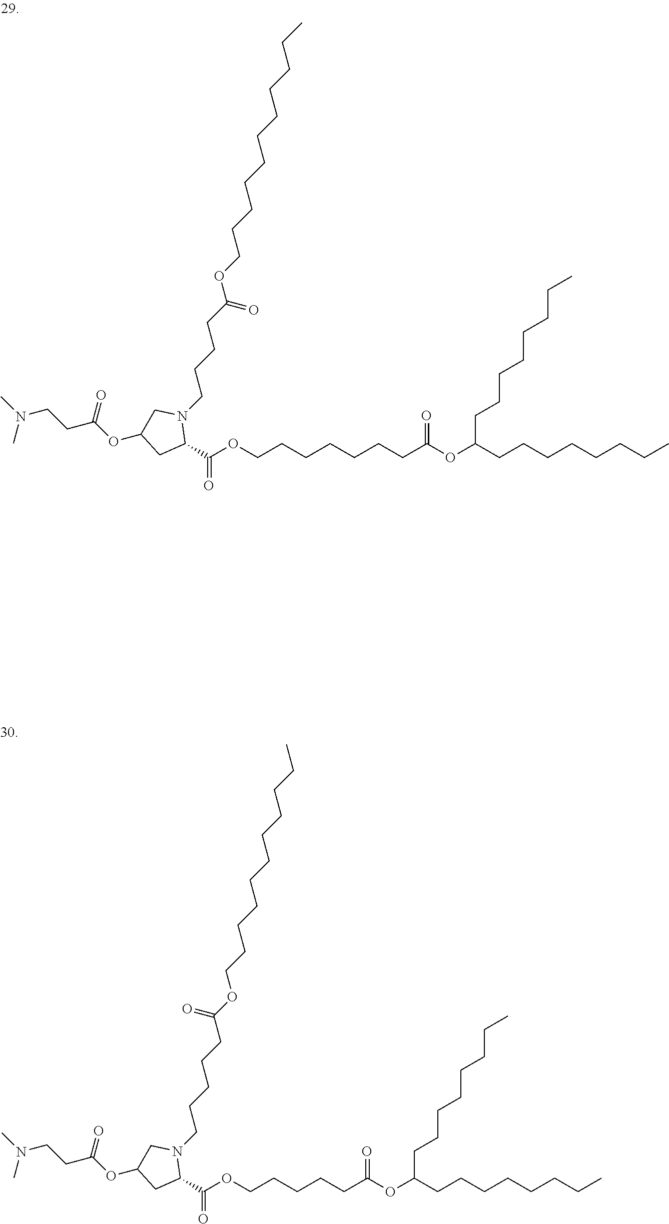

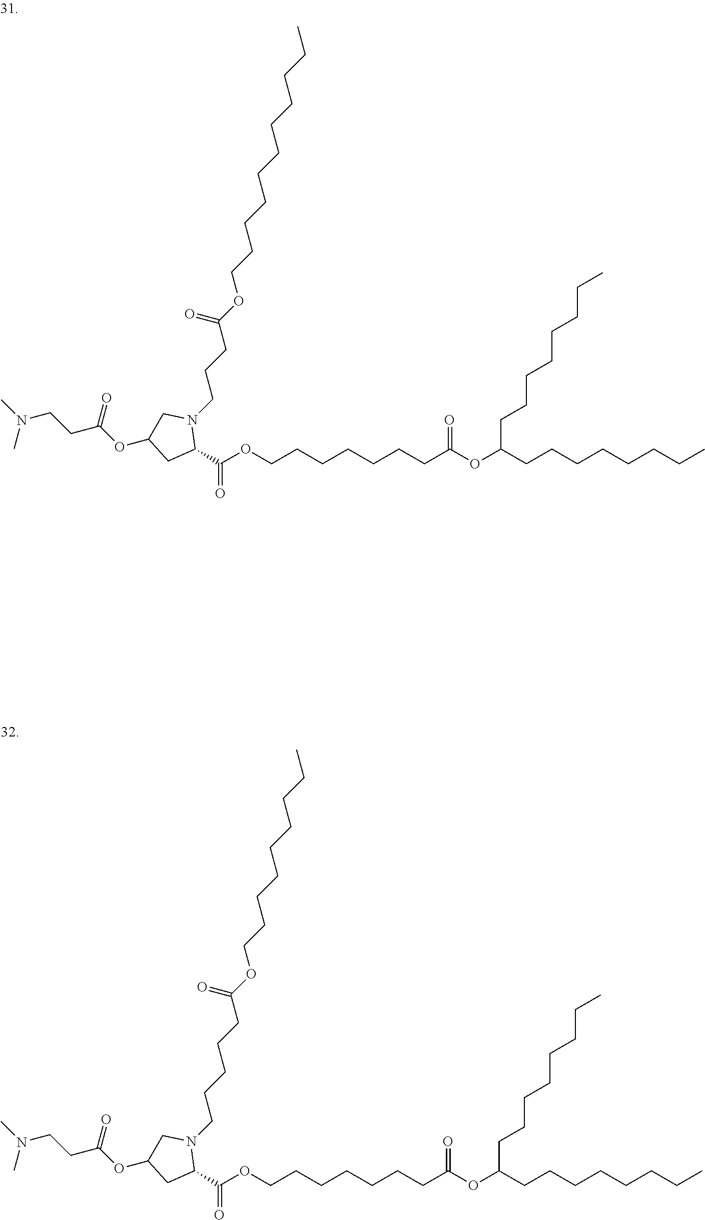

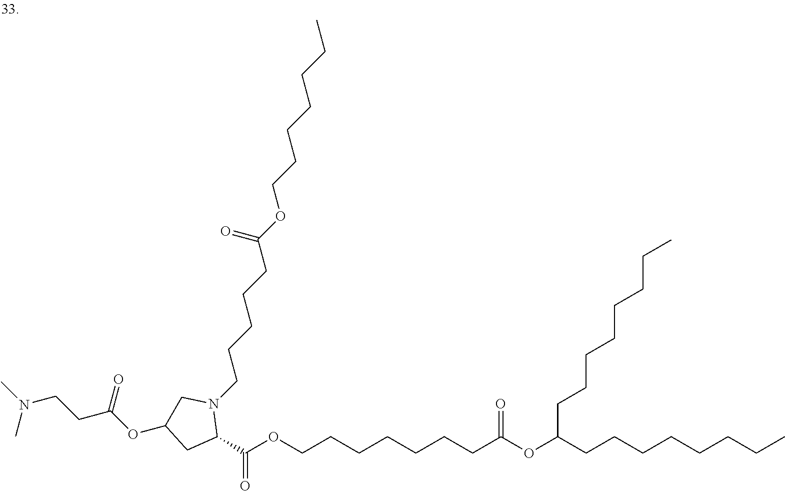 [SMILES: CCCCCCCCC(CCCCCCCC)OC(=O)CCCCCCCOC(=O)[C@@H]1CC(OC(=O)CCN(C)C)CN1CCCCCC(=O)OCCCCCCC]

39. A lipid composition comprising a compound of claim 1, wherein the lipid composition is a LNP.

40. The lipid composition of claim 39, further comprising a second lipid, optionally wherein the lipid composition comprises about a 1:1 ratio of the compound and the second lipid.

41-43. (canceled)

44. The lipid composition of claim 39, further comprising a sterol, a PEG lipid, a phospholipid, and/or a neutral lipid.

45. A pharmaceutical composition comprising the lipid composition of claim 39, a therapeutic agent, and a pharmaceutically acceptable excipient.

46. (canceled)

47. The pharmaceutical composition of claim 45, wherein the therapeutic agent is a nucleic acid molecule, a protein, or small molecule drug.

48. The pharmaceutical composition of claim 47, wherein the nucleic acid molecule is a RNA or DNA: optionally wherein the nucleic acid molecule is a RNA comprising a mRNA.

49-51. (canceled)

52. A method of delivering a therapeutic agent to a subject or a target organ of a subject, the method comprising administering to the subject the pharmaceutical composition of claim 45.

53. (canceled)

54. The method of claim 52, wherein the therapeutic agent is delivered to a target organ selected from the group consisting of pancreas, spleen, or the lung, and wherein:
less than 50%, 30%, or 10% of the therapeutic agent is delivered to the liver; and/or
more than 50%, 70%, or 90% of the therapeutic agent is delivered to the pancreas, spleen, and/or lung of the subject.

55. (canceled)
